(19) United States
(12) Patent Application Publication
(10) Pub. No.: US 2023/0035298 A1
(43) Pub. Date: Feb. 2, 2023

(54) CULTURES OF AND METHODS OF MANUFACTURING SQUAMOUS CELL CARCINOMA CELLS

(71) Applicant: THE REGENTS OF THE UNIVERSITY OF CALIFORNIA, Oakland, CA (US)

(72) Inventors: Aaron Tward, San Francisco, CA (US); Vicente Planells-Palop, San Francisco, CA (US)

(21) Appl. No.: 17/778,393

(22) PCT Filed: Nov. 19, 2020
(86) PCT No.: PCT/US2020/061395

§ 371 Date: May 19, 2022

Related U.S. Application Data
(60) Provisional application No. 62/937,787, filed on Nov. 19, 2019.

(51) Int. Cl. C12N 15/113; C12N 9/22

(52) U.S. Cl. CPC C12N 15/113; C12N 9/22; C12N 2310/20

(57) ABSTRACT

The present disclosure generally relates to a cell culturing system, and specifically to a three-dimensional cell culturing system for modified primary keratinocytes that mimic those cells of in vivo squamous cell carcinoma cells. The three-dimensional cell culturing system allows for flexibility in size and modularity of skin cancer cell growth with physiologically-relevant results.

### RELATED APPLICATIONS

This application claims the benefit of U.S. provisional Application No. 62/937,787, filed Nov. 19, 2019, the entirety of which is hereby incorporated by reference for all purposes.

### FIELD OF THE DISCLOSURE

The present disclosure generally relates to a method of generating genome-edited keratinocytes, a cell culturing system, and particularly to a cell culturing system for genome-edited keratinocytes that mimic those cells of in vivo squamous cell carcinoma cells and their progenitors. The cell culturing system of the present disclosure allows for flexibility in size and modularity of cancer cell growth with physiologically-relevant results.

### BACKGROUND

The epidermis is a stratified squamous epithelium that forms the protective covering of the skin. Only the innermost, basal layer of epidermal cells typically has the capacity for DNA synthesis and mitosis. During epidermal differentiation, a basal cell will begin its journey to the skin surface. In transit, it undergoes a series of morphological and biochemical changes that culminate in the production of dead, flattened, enucleated squames, which are sloughed from the surface, and continually replaced by inner cells differentiating outward. A representative diagram of intralayers in epithelium tissue is shown in FIG. 1.

Squamous cell carcinoma (SCC) is common form of cancer and is the second most common form of skin cancer in the United States. SCCs can affect any of the squamous cells that form the surface of the skin, line of hollow organs in the body, and line the respiratory and digestive tracts. Common types of this cancer include squamous cancers of the skin, upper aerodigestive tract, esophagus, vagina, cervix, anus, bladder, and lung. Incidence and prognosis varies by primary organ.

The evolution and progression of SCC are thought to result from multiple stepwise alterations of cellular and molecular pathways in the squamous epithelium. Mounting evidence suggests a model of molecular progression from premalignant lesions to invasive disease (FIG. 2).

Squamous cell carcinoma of the skin (or skin SCC) is a common form of skin cancer that develops in the squamous cells that make up the middle and outer layers of the skin. Skin SCC affects 660,000 people per year and generally carries an excellent prognosis. For a very small skin SCC that has a low risk of spreading, less invasive treatments, including curettage and electrodessication (C and E), laser therapy, freezing, and photodynamic therapy, may be used. For a larger skin SCC that extends deeper into the skin, more invasive treatments, such as simple excision, mohs surgery, or radiation therapy, are recommended. For a skin SCC that spreads beyond the skin, drug treatments, including chemotherapy, targeted drug therapy, and immunotherapy, are recommended.

Currently, there are only two drugs have been approved specifically for treatment of SCCs. Moreover, for the upper aerodigestive tract, SCC is by far the most common malignancy and the median five-year survival rate for this type of SCC is approximately 50% and has only improved minimally over the past 50 years. Thus, additional therapeutics and treatments are still needed for squamous cell carcinomas, particularly for those that are not ineligible for surgical interventions and/or unresponsive to radiotherapy and chemotherapy.

### SUMMARY OF THE EMBODIMENTS

In one aspect, the present disclosure relates to a method of genetically altering one or a plurality of primary keratinocytes comprising exposing the one or plurality of primary keratinocytes to one or a plurality of mutagens for a time period sufficient to mutate one or a plurality of wild-type genes such that one or a plurality of genome-edited primary keratinocytes deficient in expression of the one or plurality of wild-type genes are produced. In some embodiments, the one or plurality of wild-type genes being mutated are chosen from Table 1 provided in the present disclosure.

In some embodiments, the one or plurality of mutagens is a vector or a gene-modifying enzyme. In some embodiments, the gene-modifying enzyme is a Cas protein, a meganuclease, a transcription activator-like effector nucleases (TALEN), a transposon, a zinc-finger nuclease (ZFN), or a recombinase.

In some embodiments, the method of the present disclosure further comprises transfecting the one or plurality of primary keratinocytes with a plasmid comprising a nucleic acid sequence encoding the one or plurality of mutagens. In some embodiments, the nucleic acid sequence encodes a Cas9 protein. In some embodiments, the plasmid further comprises one or a plurality of small guide RNAs guiding the Cas9 protein to the one or plurality of wild-type genes for genome editing.

In some embodiments, the method of the present disclosure further comprises a step of selecting the one or plurality of genome-edited primary keratinocytes deficient in expression of the one or plurality of wild-type genes. In some embodiments, the step of selecting is performed based on expression of a marker gene. In some embodiments, the marker gene is a fluorescent reporter gene, an antibiotic resistance gene, or a surface protein-coding gene. In some embodiments, the fluorescent reporter gene encodes a green fluorescent protein (GFP), a tdTomato fluorescent protein, an EBFP2 fluorescent protein, a mKate2 fluorescent protein, or a mKO fluorescent protein. In some embodiments, the antibiotic resistance gene is an ampicillin resistance gene, a kanamycin resistance gene, or a puromycin resistance gene. In some embodiments, the surface protein-coding gene encodes a kinase domain deleted low-affinity NGF receptor (DeltaLNGFR) coupled with a streptavidin binding peptide (SBP).

In some embodiments, the one or plurality of genome-edited primary keratinocytes are deficient in expression of at least one of wild-type TP53, CDKN2A, and NOTCH1. In some embodiments, the one or plurality of genome-edited primary keratinocytes are deficient in expression of wild-type TP53, CDKN2A, and NOTCH1.

In another aspect, the present disclosure relates to a method of generating a population of one or a plurality of genome-edited keratinocytes comprising:
a) generating and selecting one or a plurality of genome-edited primary keratinocytes deficient in expression of one or a plurality of wild-type genes according to any of the embodiments disclosed herein;
b) expanding the one or plurality of genome-edited primary keratinocytes into the population of one or plurality of genome-edited keratinocytes.

In some embodiments, the one or plurality of primary keratinocytes used in any of the methods disclosed herein are primary human keratinocytes.

In a further aspect, the present disclosure relates to a composition comprising the population of one or plurality of genome-edited keratinocytes generated by any of the methods disclosed herein. In some embodiments, the population of one or plurality of genome-edited keratinocytes is deficient in expression of one or a plurality of wild-type genes chosen from Table 1. In some embodiments, the population of one or plurality of genome-edited keratinocytes is deficient in expression of at least one of wild-type TP53, CDKN2A, and NOTCH1. In some embodiments, the population of one or plurality of genome-edited keratinocytes is deficient in expression of wild-type TP53, CDKN2A, and NOTCH1.

In yet another aspect, the present disclosure relates to a composition comprising one or a plurality of genome-edited keratinocytes that is deficient in expression of one or a plurality of wild-type genes chosen from Table 1. In some embodiments, the population of one or plurality of genome-edited keratinocytes is deficient in expression of at least one of wild-type TP53, CDKN2A, and NOTCH1. In some embodiments, the one or plurality of genome-edited keratinocytes are deficient in expression of wild-type TP53, CDKN2A and NOTCH1. In some embodiments, the keratinocytes are primary human keratinocytes.

In some embodiments, the disclosure relates to a composition comprising one or a plurality of cells, particularly keratinocytes, comprising one or a plurality of mutations in a gene comprising at least about 70%, 75%, 80%, 85%, 90%, 91%, 92%, 93%, 94%, 95%, 96%, 97%, 98%, 99%, or 100% sequence identity to SEQ ID NO: 307. In some embodiments, the disclosure relates to a composition comprising one or a plurality of cells, particularly keratinocytes, comprising one or a plurality of mutations in a gene comprising at least about 70%, 75%, 80%, 85%, 90%, 91%, 92%, 93%, 94%, 95%, 96%, 97%, 98%, 99%, or 100% sequence identity to SEQ ID NO: 308. In some embodiments, the disclosure relates to a composition comprising one or a plurality of cells, particularly keratinocytes, comprising one or a plurality of mutations in a gene comprising at least about 70%, 75%, 80%, 85%, 90%, 91%, 92%, 93%, 94%, 95%, 96%, 97%, 98%, 99%, or 100% sequence identity to SEQ ID NO: 309. In some embodiments, the disclosure relates to a composition comprising one or a plurality of cells, particularly keratinocytes, comprising one or a plurality of mutations in a gene comprising at least about 70%, 75%, 80%, 85%, 90%, 91%, 92%, 93%, 94%, 95%, 96%, 97%, 98%, 99%, or 100% sequence identity to SEQ ID NO: 307. In some embodiments, the disclosure relates to a composition comprising one or a plurality of cells, particularly keratinocytes, comprising one or a plurality of mutations in each of the genes comprising at least about 70%, 75%, 80%, 85%, 90%, 91%, 92%, 93%, 94%, 95%, 96%, 97%, 98%, 99%, or 100% sequence identity to SEQ ID NO: 307 and SEQ ID NO: 308. In some embodiments, the disclosure relates to a composition comprising one or a plurality of cells, particularly keratinocytes, comprising one or a plurality of mutations in each of the genes comprising at least about 70%, 75%, 80%, 85%, 90%, 91%, 92%, 93%, 94%, 95%, 96%, 97%, 98%, 99%, or 100% sequence identity to SEQ ID NO: 307 and SEQ ID NO: 309. In some embodiments, the disclosure relates to a composition comprising one or a plurality of cells, particularly keratinocytes, comprising one or a plurality of mutations in each of the genes comprising at least about 70%, 75%, 80%, 85%, 90%, 91%, 92%, 93%, 94%, 95%, 96%, 97%, 98%, 99%, or 100% sequence identity to SEQ ID NO: 308 and SEQ ID NO: 309. In some embodiments, the disclosure relates to a composition comprising one or a plurality of cells, particularly keratinocytes, comprising one or a plurality of mutations in each of the genes comprising at least about 70%, 75%, 80%, 85%, 90%, 91%, 92%, 93%, 94%, 95%, 96%, 97%, 98%, 99%, or 100% sequence identity to SEQ ID NO: 307, SEQ ID NO: 308, and SEQ ID NO: 309.

In some embodiments, the disclosure relates to a composition comprising one or a plurality of cells, particularly keratinocytes, comprising one or a plurality of mutations in a gene encoding a polypeptide comprising at least about 70%, 75%, 80%, 85%, 90%, 91%, 92%, 93%, 94%, 95%, 96%, 97%, 98%, 99%, or 100% sequence identity to SEQ ID NO: 347. In some embodiments, the disclosure relates to a composition comprising one or a plurality of cells, particularly keratinocytes, comprising one or a plurality of mutations in a gene encoding a polypeptide comprising at least about 70%, 75%, 80%, 85%, 90%, 91%, 92%, 93%, 94%, 95%, 96%, 97%, 98%, 99%, or 100% sequence identity to SEQ ID NO: 348. In some embodiments, the disclosure relates to a composition comprising one or a plurality of cells, particularly keratinocytes, comprising one or a plurality of mutations in a gene encoding a polypeptide comprising at least about 70%, 75%, 80%, 85%, 90%, 91%, 92%, 93%, 94%, 95%, 96%, 97%, 98%, 99%, or 100% sequence identity to SEQ ID NO: 349. In some embodiments, the disclosure relates to a composition comprising one or a plurality of cells, particularly keratinocytes, comprising one or a plurality of mutations in each of the genes encoding polypeptides comprising at least about 70%, 75%, 80%, 85%, 90%, 91%, 92%, 93%, 94%, 95%, 96%, 97%, 98%, 99%, or 100% sequence identity to SEQ ID NO: 347 and SEQ ID NO: 348. In some embodiments, the disclosure relates to a composition comprising one or a plurality of cells, particularly keratinocytes, comprising one or a plurality of mutations in each of the genes encoding polypeptides comprising at least about 70%, 75%, 80%, 85%, 90%, 91%, 92%, 93%, 94%, 95%, 96%, 97%, 98%, 99%, or 100% sequence identity to SEQ ID NO: 347 and SEQ ID NO: 349. In some embodiments, the disclosure relates to a composition comprising one or a plurality of cells, particularly keratinocytes, comprising one or a plurality of mutations in each of the genes encoding polypeptides comprising at least about 70%, 75%, 80%, 85%, 90%, 91%, 92%, 93%, 94%, 95%, 96%, 97%, 98%, 99%, or 100% sequence identity to SEQ ID NO: 348 and SEQ ID NO: 349. In some embodiments, the disclosure relates to a composition comprising one or a plurality of cells, particularly keratinocytes, comprising one or a plurality of mutations in each of the genes encoding polypeptides comprising at least about 70%, 75%, 80%, 85%, 90%, 91%, 92%, 93%, 94%, 95%, 96%, 97%, 98%, 99%, or 100% sequence identity to SEQ ID NO: 347, SEQ ID NO: 348 and SEQ ID NO: 349.

In some embodiments, the composition of the present disclosure further comprises a top layer of cells and a bottom layer of tissue, the bottom layer of tissue comprising extracellular matrix protein and one or multiple cell types and the top layer of cells comprising the one or plurality of genome-edited keratinocytes. In some embodiments, the cells of the bottom layer of tissue comprises one or a plurality of fibroblasts. In some embodiments, the cells of the bottom layer of tissue comprise one or a plurality of a combination of cells including fibroblasts, endothelium, CD45+ immune cells, pericytes, and/or myoepithelial cells. In some embodiments, the composition of the present disclosure further comprises a hydrogel layer positioned underneath the bottom layer of tissue. In some embodiments, the hydrogel layer comprises one or a plurality of growth factors or functional fragments thereof. In some embodiments, the one or plurality of growth factors are chosen from Table 4 provided in the present disclosure or a functional fragment comprising at least about 70%, 75%, 80%, 85%, 90%, 91%, 92%, 93%, 94%, 95%, 96%, 97%, 98%, 99%, or 100% sequence identity to the amino acid sequence of the growth factors listed in Table 4 of the present disclosure. In some embodiments, the hydrogel is Matrigel®.

In some embodiments, the composition of the present disclosure is at 37 degrees Celsius and exposed to about 5% CO2 for no less than 24 or 48 hours.

In some embodiments, the composition of the present disclosure further comprises at least a single insert comprising a biocompatible polymer and an inner wall defining an inner volume divided into an upper chamber and a bottom chamber, the upper chamber and bottom chamber positioned adjacently to one another and in fluid communication and separated by a ledge; the ledge positioned along an inner wall of the insert and defining a planar interface between the upper and bottom chambers. In some embodiments, the composition comprises a top layer of cells, a bottom layer of tissue and a hydrogel; each of the top layer of cells and bottom layer of tissue positioned in the upper chamber; and wherein the hydrogel is positioned in the bottom chamber; wherein the bottom layer of tissue comprises extracellular matrix protein and the top layer of cells comprises the one or plurality of genome-edited keratinocytes keratinocytes disclosed herein. In some embodiments, the top layer of cells comprises comprise one or a plurality of cells including fibroblasts, endothelial cells, CD45+ immune cells, pericytes, melanocytes. Merkel cells, and Langerhans cells, and/or myoepithelial cells. In some embodiments, the composition comprises at least about 2, 6, 12, 24, 48, 96, 192 or more inserts. In some embodiments, the composition comprises at least about 2, 6, 12, 24, 48, 96, 192 or more inserts, each insert comprising a space or opening or interface at the top of the insert separating the insert from a point exterior to the insert. In some embodiments, the interface is a planar or substantially planar opening with a surface area from about 0.1 to about 5 square centimeters. In some embodiments, the upper chamber has a volume from about 0.1 to about 9 centimeters cubed. In some embodiments, the bottom chamber has a volume from about 0.1 to about 9 centimeters cubed.

In some embodiments, the composition of the present disclosure is frozen at from about −20 to about −212 degrees Celsius.

In another aspect, the present disclosure relates to a system comprising:
a) at least one solid support comprising a bottom surface and sidewalls, the bottom surface and sidewalls defining a culture volume;
b) at least one insert as defined herein positioned on the bottom surface of the at least one solid support; and
c) one or a plurality of keratinocytes that is deficient in expression of one or a plurality of wild-type genes chosen from Table 1 positioned within the upper chamber of the insert.
   In some embodiments, the one or plurality of keratinocytes are deficient in expression of at least one of wild-type TP53, CDKN2A, and NOTCH1. In some embodiments, the one or plurality of keratinocytes are deficient in expression of wild-type TP53, CDNK2A, and NOTCH1. In some embodiments, the one or plurality of keratinocytes are deficient in expression of wild-type TP53, CDKN2A, and NOTCH1 in combination with mutations in other genes. In some embodiments, the keratinocytes are primary human keratinocytes.

In some embodiments, the system of the present disclosure further comprises a top layer of cells and a bottom layer of tissue, the bottom layer of tissue comprising extracellular matrix protein and the top layer of cells comprising the one or plurality of keratinocytes. In some embodiments, the bottom layer of tissue comprises one or a plurality of fibroblasts. In some embodiments, the system of the present disclosure further comprises a hydrogel layer positioned adjacently underneath the bottom layer of tissue. In some embodiments, the hydrogel layer comprises one or a plurality of growth factors or functional fragments thereof. In some embodiments, the one or plurality of growth factors are chosen from Table 4 provided in the present disclosure or a functional fragment comprising at least about 70%, 75%, 80%, 85%, 90%, 91%, 92%, 93%, 94%, 95%, 96%, 97%, 98%, 99%, or 100% sequence identity to the amino acid sequence of the growth factors listed in Table 4 of the present disclosure. In some embodiments, the hydrogel is Matrigel®.

In some embodiments, the system of the present disclosure further comprises an incubator in which the solid support is positioned and wherein the incubator comprises a heating element and an air inlet through which air content is controlled. In some embodiments, the temperature is about 37 degrees Celsius and comprises ambient air in contact with the solid support that comprises about 5% carbon dioxide.

In some embodiments, the system of the present disclosure further comprises culture media in the volume of the solid support with a cell culture media volume sufficient to cover the solid support but exposing one or plurality of keratinocytes to air. In some embodiments, the system of the present disclosure further comprises a tissue culture medium at a volume sufficient to cover the bottom tissue and create an air liquid interface immediately adjacent to or below the top layer of cells, such that the top layer of cells is exposed to air. In some embodiments, the air is filtered or sterilized in a closed system.

In some embodiments, the solid support in the system of the present disclosure comprises one or a plurality of wells or inserts from about 3 to about 5 centimeters in height. In some embodiments, the system comprises a solid support that is from about 1 to about 10 centimeters in height.

In some embodiments, the system of the present disclosure comprises a top layer of cells, a bottom layer of tissue and a hydrogel; each of the top layer of cells and bottom layer of tissue positioned in the upper chamber; and wherein the hydrogel positioned in the bottom chamber; wherein the bottom layer of tissue comprises extracellular matrix protein and the top layer of cells comprises the one or plurality of primary keratinocytes. In some embodiments, the system of the present disclosure comprises at least about 6, 12, 24, 48, 96, 192 or more inserts.

In some embodiments, the interface in the system of the present disclosure is a planar or substantially planar opening with a surface area from about 0.1 to about 5 square centimeters. In some embodiments, the upper chamber in the system of the present disclosure has a volume from about 0.1 to about 9 centimeters cubed. In some embodiments, the bottom chamber in the system of the present disclosure has a volume from about 0.1 to about 9 centimeters cubed.

In some embodiments, the system of the present disclosure further comprises a tissue culture medium at a volume sufficient to cover the bottom tissue and create an air liquid interface immediately adjacent to or below the top layer of cells, such that the top layer of cells is exposed to air. In some embodiments, the air is filtered or sterilized in a closed system.

In some embodiments, the system of the present disclosure further comprises a gas reservoir in fluid communication with the solid support via one or a plurality of gas lines; and a heating element operably positioned at or proximate to the solid support, such that the temperature of ambient air around the top layer of cells is from about 32 to about 42 degrees Celsius.

In some embodiments, the system of the present disclosure further comprises one or more therapeutic compounds.

In another aspect, the present disclosure provides a method of isolating a primary keratinocytes from skin tissue or mucosa comprising culturing a primary keratinocyte to culture medium while simultaneously exposing one or more keratinocytes to one or a plurality of tissue samples comprising extracellular matrix (ECM). In some embodiments, the skin tissue is obtained from any part of a human body. In some embodiments, the skin is anal skin. In some embodiments, the mucosa is oral mucosa, mucosa of the upper aerodigestive tract, esophageal mucosa, vaginal mucosa, or cervical mucosa.

In a further aspect, the present disclosure relates to a method of culturing a primary keratinocyte comprising exposing the primary keratinocyte to a system comprising:
a) at least one solid support comprising a bottom surface and sidewalls, the bottom surface and sidewalls defining a culture volume; and
b) at least one insert as defined herein positioned on the bottom surface of the at least one solid support; wherein the insert comprises a layer of tissue comprising ECM onto which the primary keratinocytes are positioned.

In some embodiments, the step of exposing further comprises exposing the primary keratinocyte to cell culture media comprising:

a) DMEM/HAM F12(3:1) supplemented with 10% FBS;

b) 1% penicillin/streptomycin;

c) 1% antibiotic-antimycotic;

d) 3 μg/mL of adenine;

e) 3 ng/mL of cholera toxin; 40 ng/mL of hydrocortisone;

g) 5 μg/mL of insulin;

h) 10 ng/mL of EGF;

i) 5 μg/mL of Transferrin;

j) 1.5 ng/mL of triiodo-L-thyronine; and

k) 10 ng/mL of ciprofloxacin hydrochloride,

In some embodiments, the step of exposing further comprises exposing the primary keratinocyte to cell culture media comprising:

a) DMEM/HAM F12(3:1) supplemented with about 10% FBS;

b) about 1% penicillin/streptomycin;

c) about 1% antibiotic-antimycotic;

d) about 3 μg/mL of adenine;

e) about 3 ng/mL of cholera toxin;

about 40 ng/mL of hydrocortisone;

g) about 5 μg/mL of insulin;

h) about 10 ng/mL of EGF;

i) about 5 μg/mL of Transferrin;

j) about 1.5 ng/mL of triiodo-L-thyronine; and

k) about 10 ng/mL of ciprofloxacin hydrochloride,

for a time period sufficient to propagate the one or plurality of primary keratinocytes for no less than about one day. In some embodiments, the exposing step comprising exposing the primary keratinocyte to cell culture media comprising:

a) DMEM/HAM F12(3:1) supplemented with 10% FBS;

b) 1% penicillin/streptomycin;

c) 1% antibiotic-antimycotic;

d) 3 μg/mL of adenine;

e) 3 ng/mL of cholera toxin;

f) 40 ng/mL of hydrocortisone;

g) 5 μg/mL of insulin;

h) 10 ng/mL of EGF;

i) 5 μg/mL of Transferrin;

j) 1.5 ng/mL of triiodo-L-thyronine; and

k) 10 ng/mL of ciprofloxacin hydrochloride,

for a time period sufficient to propagate the one or plurality of primary keratinocytes for no less than about three days. In some embodiments, the exposing step comprising exposing the primary keratinocyte to cell culture media comprising:

a) DMEM/HAM F12(3:1) supplemented with 10% FBS;

b) about 1% penicillin/streptomycin;

c) about 1% antibiotic-antimycotic;

d) about 3 μg/mL of adenine;

e) about 3 ng/mL of cholera toxin;

about 40 ng/mL of hydrocortisone;

g) about 5 μg/mL of insulin;

h) about 10 ng/mL of EGF;

i) about 5 μg/mL of Transferrin;

j) about 1.5 ng/mL of triiodo-L-thyronine; and

k) about 10 ng/mL of ciprofloxacin hydrochloride,

for a time period sufficient to propagate the one or plurality of primary keratinocytes for no less than about seven days. In some embodiments, the step of exposing further comprises positioning a layer of hydrogel underneath the layer of tissue comprising ECM.

In some embodiments, the method of the present disclosure further comprises a step of exposing the one or plurality of primary keratinocytes to one or a plurality of therapeutics. In some embodiments, the therapeutic is a nucleic acid molecule, a peptide or a small molecule, or any combination thereof.

In some embodiments, the method of the present disclosure further comprises monitoring morphology of the one or plurality of primary keratinocytes in response to exposure to the one or plurality of therapeutics.

In some embodiments, the culture system in the method of the present disclosure comprises from about 10 to about 100 inserts.

In another aspect, the present disclosure relates to a method of screening a compound for biological activity comprising exposing one or a plurality of compounds to one or a plurality of genome-edited keratinocytes comprised in any of the compositions disclosed herein.

### BRIEF DESCRIPTION OF THE DRAWINGS

FIG. 1 shows a representative diagram of intralayers in epithelium tissue.

FIG. 2 shows a model of molecular progression from premalignant lesions to invasive disease.

FIG. 3 shows a schematic of the genome-editing by CRISPR/cas9 transfection followed by FACS sorting based on GFP expression.

FIG. 4A shows a schematic of the pSpCas9(BB)-2A-GFP vector map and FIG. 4B shows a schematic of linearized pSpCas9(BB)-2A-GFP vector used in Example 1.

FIG. 5 shows the percentage of GFP positive cells detected after up to 64 hours post-transfection.

FIG. 6 shows the percentage of primary keratinocytes showing genome-editing 24 hours after transfection.

FIG. 7 shows the genome editing efficiency measured by TIDE before and after FACS selection. TIDE results showing genome-editing efficiency (percentage of cells harboring mutations in the FAT1 gene loci) for non-sorted (top) and sorted (bottom) populations.

FIG. 8 shows the percentage of genome-edited keratinocytes in multiple conditions (in each condition, one single gene was targeted with CRISPR/Cas9) over different passages.

FIG. 9 shows the recovery of genome-edited keratinocytes harboring one single mutation in the genome following FACS-selection.

FIG. 10 shows a schematic representation of a stand design, including the 3D view, top view, side view, and front view.

FIG. 11 shows a ratio of ledge surface and cavity area of a stand from a top view.

FIG. 12 shows the organotypic culture protocol.

FIG. 13 shows the structure of the organotypic cultures of certain genome-edited keratinocytes containing multiple mutations.

FIG. 14A shows the image obtained from squamous cell carcinoma tumor and FIG. 14B shows the structure of the TP53+CDKN2A+NOTCH1 mutated keratinocytes after 2, 3 or 4 weeks of organotypic culture.

FIG. 15A-15F show that single gene mutation is not sufficient for keratinocyte transformation. FIG. 15A: Significantly altered genes with loss of function mutations in head and neck squamous cell carcinoma (TCGA). Columns represent individual tumors; Gray dots, point mutations; light gray bars, gene deletion; dark gray bars, gene amplification. FIG. 15B: Percentage of primary human keratinocytes harboring mutations in specific genes, 24 hours post-transfection (p<0.05 for all individual results). FIG. 15C: Percentage of primary human keratinocytes harboring mutations in a single gene over multiple passages. One representative experiment shown from N=2 biological replicates. FIG. 15D: Organotypic skin culture of genome-edited primary human keratinocytes. FIG. 15E: Total number of cells recovered following FACS-selection of genome-engineered keratinocytes. FIG. 15F: Fraction of primary human tumors harboring gene deletions and mutations in TP53, CDKN2A and NOTCH1 in SCC from distinct tissues indicated (7-13).

FIG. 16 shows the number of driver genomic alterations per HNSCC tumor. Histogram depicting the number of HNSCC — TCGA cases (N=496) with genomic alterations (point mutations, deletions or amplifications) in the genes included in this study. Dotted line represents the trend.

FIG. 17A-17D show genomic alterations in postulated drivers of HNSCC. FIG. 17A: Percentage of cases showing genomic alterations (point mutations and deletions) in specified genes. FIG. 17B and FIG. 17C: Percentage of cases showing genomic alterations (point mutations and deletions) in specified genes in addition to alterations in TP53 or TP53 and CDKN2A, respectively. FIG. 17D: Venn diagram depicting tumors with TP53, CDKN2A and NOTCH1 alterations in HNSCC.

FIG. 18A-18E show that TP53, CDKN2A, and NOTCH1 mutations in combination are sufficient for transformation. FIG. 18A: Percentage of primary human keratinocytes harboring mutations in indicated genes over multiple passages. One representative experiment shown from N=3 biological replicates. FIG. 18B: Total number of cells recovered following FACS-selection and passaging of genome-engineered keratinocytes indicating immortalization. FIG. 18C: Percentage of primary human keratinocytes harboring mutations in specific genes for the expanded conditions (p<0.05 for all individual results). FIG. 18D: Total cell numbers from a time course of genome-edited keratinocytes harboring specific mutations. Data points represents mean±s.e.m, n=3 samples per condition and day. Asterisk represents p value <0.05. FIG. 18E: Sphere-forming assay. Data points represent sphere forming frequency±s.d. (n=6 per group). Asterisk represents p value <0.05.

FIG. 19A-19B show cell cycle analysis of genome-edited keratinocytes. FIG. 19A: Pictures of genome-edited keratinocytes taken after 5 days in 2D culture. FIG. 19B: Cell cycle analysis of genome-edited keratinocytes. Data points represents mean±standard deviation, n=3 samples per condition. Asterisk represents p value <0.05.

FIG. 20A-20D show that TP53, CDKN2A, and NOTCH1 mutations drive keratinocyte invasion. FIG. 20A: H&E staining of organotypic cultures performed with genome-edited keratinocytes. The dotted line indicates the location of the basement membrane. FIG. 20B: Immunohistochemical staining showing K167 and P40 markers on organotypic cultures performed with genome-edited keratinocytes. The dotted line indicates the location of the basement membrane. FIG. 20C: H&E staining of organotypic cultures performed with CTN^{mut} keratinocytes, incubated for 3 weeks. The dotted line depicts the location of the basement membrane. FIG. 20D: H&E staining and P40 immunostaining of tumor section from an NSG mouse injected with CTN^{mut} keratinocytes.

FIG. 21 shows genome-edited foreskin keratinocytes. H&E staining of organotypic cultures performed with CTN^{mut} keratinocytes, incubated for 2-4 weeks. Black arrowheads indicate invasive cells. Gray arrowhead indicates keratin pearl. Dotted line depicts the location of the basement membrane.

FIG. 22A-22D show in vivo genome-edited cells injection. FIG. 22A: Genome-edited keratinocytes (CT^{mut} and CTN^{mut} cells) were injected in the flank of NSG mice. 3.5 months after injection, CTN^{mut} cells formed tumors. Bottom panels shows tumor formed by CTN^{mut} keratinocytes. FIG. 22B: Kaplan-Meier plot showing survival rate of mice injected with CT^{mut} and CTN^{mut} cells. FIG. 22C: Percentage of cells from tumors showing alterations in specific genes. FIG. 22D: H&E and p40 immunohistochemical staining of CTN^{mut} keratinocytes-derived tumor.

FIG. 23 shows UCSF500 copy number analysis of genome-edited keratinocytes. Genome-wide copy number analysis of genome-edited keratinocytes.

FIG. 24 shows genome-edited oral (tonsil) keratinocytes. Organotypic cultures with genome-engineered primary human oral keratinocytes. The dotted line depicts the location of the basement membrane. Arrowheads indicate invasive cells.

FIG. 25A-25C show genetic drivers of transformed-keratinocyte invasion. FIG. 25A: Unsupervised hierarchical clustering analysis of transcriptomic variations across genome-engineered keratinocytes. N=3 replicates per condition, except for CT^{mut} when N=2. FIG. 25B: Alterations in the expression of different cellular processes markers. FIG. 25C: Differentially expressed genes (log 2 fold change) between CT^{mut} and CTN^{mut} primary keratinocytes.

FIG. 26A-26B show RNA-seq analysis of genome-edited keratinocytes. RNA-seq analyses of genome-edited keratinocytes including (FIG. 26A) log 2 fold change in gene expression (FIG. 26B) gene-ontology analyses (performed with PANTHER^{35,36}). Left, CDKN2A^{mut} keratinocytes; center, CT^{mut} keratinocytes; and right, CTN^{mut} keratinocytes.

FIG. 27 shows a model of squamous cell carcinoma formation and progression.

FIG. 28A-28B shows prevalence of mutations in TP53, CDKN2A and NOTCH1 in normal skin. FIG. 28A: Number of biopsies showing non-synonymous alterations in TP53, CDKN2A and NOTCH1^{17,18}. FIG. 28B: Variant allele fraction (VAF) for each mutation in the 4 biopsies from esophageal tissue study¹⁸ that show presence of mutations in TP53, CDKN2A and NOTCH1.

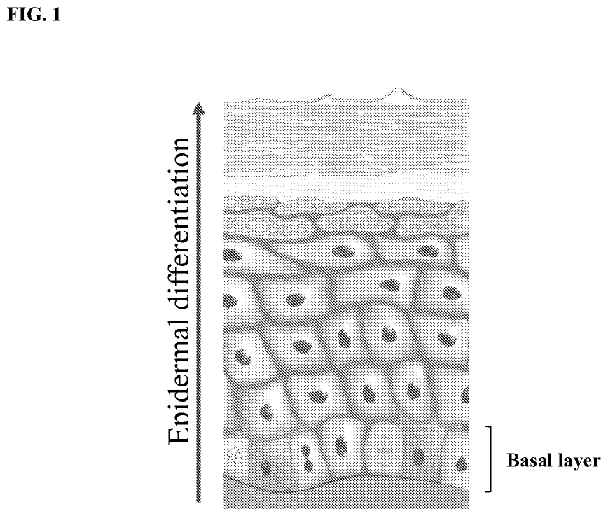

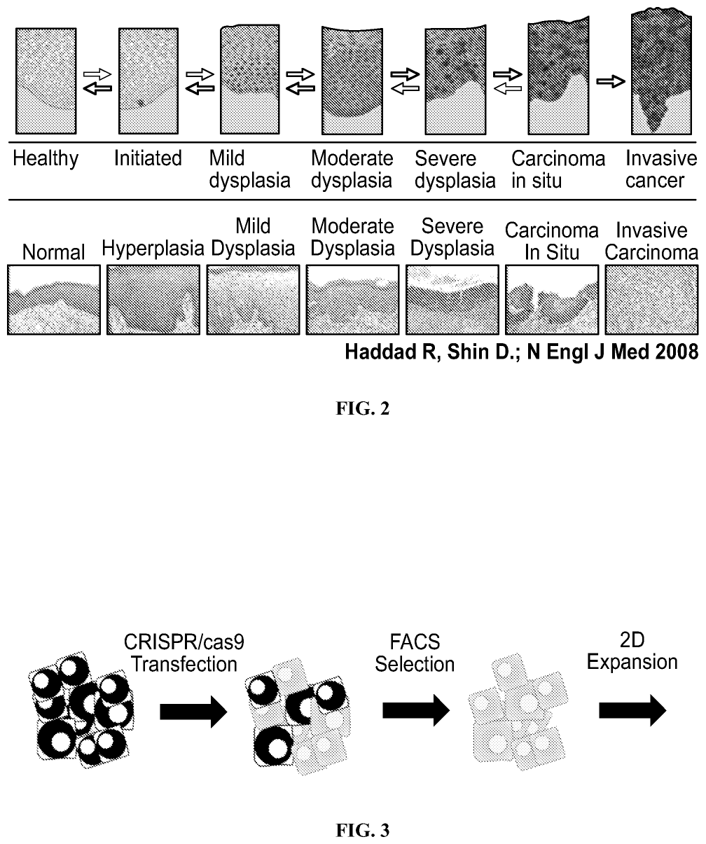

### DETAILED DESCRIPTION OF EMBODIMENTS

The present disclosure can be understood more readily by reference to the following detailed description of embodiments, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

Unless defined otherwise, all technical and scientific terms used herein have the same meaning as commonly understood by one of ordinary skill in the art to which the invention pertains.

As used in the specification and in the claims, the term “comprising” can include the aspects “consisting of” and “consisting essentially of” Comprising can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and also includes any combination of members of that list.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

As used herein, the term “primary” cell type means that the cells are derived directly from tissue comprising the named cells. In some embodiments, the cells may be human or non-human animal cells. In some embodiments, the cells are primary human, non-human primate, dog, pig, horse, cow, sheep, goat, cat, mouse, rat cells. In some embodiments, the primary cells are derived from a human, non-human primate, dog, pig, horse, cow, goat, sheep, cat, mouse, rat or other non-human animal.

As used herein, the term “deficient in expression” encompasses deficient in protein function (partial or complete loss of function), with altered function as compared to wild-type expression, or with gain of function. In some embodiments, the cells deficient in expression carrying the identified gene do not express a sufficient number of wild-type amino acid sequences to confer a wild-type biological function. In some embodiments, the cells deficient in expression are free of one or more of the identified genes. In some embodiments, the cells deficient in expression are free of one or more of the identified wild-type genes. In some embodiments, the cells deficient in expression are free of a portion or the full coding region of one or more of the wild-type TP53, CDKN2A and NOTCH1. In some embodiments, the cells deficient in expression express only a non-biologically functional or mutated form of the peptide encoded by the identified nucleic acid sequence. In some embodiments, the cells deficient in expression express one or more amino acid sequences encoded by a nucleic acid comprising at least about 70%, 75%, 80%, 85%, 90%, 95%, 96%, 97%, 98%, 99% or more sequence identity to one or a combination of: SEQ ID NO: 311, SEQ ID NO:312, SEQ ID NO:313, SEQ ID NO:314, SEQ ID NO:315 or 316, provided that the nucleic acid does not comprise the nucleic acid sequence of SEQ ID NO: 307, SEQ ID NO: 308, SEQ ID NO:309.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001.

As used herein, the terms “genetically altering,” “genome-edited,” or “genome editing” refers to genetic alternation(s) of one or a plurality of wild-type genes in animal and plant cells, including human cells, which may be knock-out or knock-in of a specific gene, or introducing a mutation into a non-coding DNA sequence that does not produce protein. The term “genome-edited” may include deletion, duplication, inversion, replacement or rearrangement of genomic DNA. In some embodiments, only a single wild-type gene is genetically altered or edited in the cell. In some embodiments, more than one, such as 2, 3, 4, 5, or more regions of one gene or a plurality of genes are altered or edited in the cell. Techniques for genome editing or introducing a targeted mutation into the nucleotide sequence of a gene are known in the art, including but not limited to the use of mutagens.

As used herein, the term “mutagen” means any energenic wave, any molecule, nucleic acid sequence, amino acid sequence, or hybrid amino acid or nucleic acid sequence that causes a mutation or modification in one or more regions of endogenous nucleic acid when exposed for a time period sufficient to cause the mutation. In some embodiments, the mutation is a point mutation, frameshift mutation, deletion, truncation, or addition. In some embodiments, the mutagen is a vector or a gene-modifying enzyme.

The term “vector” as used herein refers to any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. Vectors can be delivered into cells ex vivo by any methods known in the art, including but not limited to, non-viral transfection, biolistics, chemical transfection, electroporation, nucleofection, heat-shock transfection, lipofection, microinjection, and viral transfection.

The terms “transfection,” “transduction,” “transfecting” or “transducing” can be used interchangeably and are defined as a process of introducing a nucleic acid molecule and/or a protein to a cell. Nucleic acids may be introduced to a cell using viral-based or non-viral methods. The nucleic acid molecule can be a sequence encoding complete proteins or functional portions thereof. Typically, a nucleic acid vector, comprising the elements necessary for protein expression (e.g., a promoter, transcription start site, etc.), is used. For viral-based methods, any useful viral vector can be used in the methods described herein. Examples of viral vectors include, but not limited to, retroviral, adenoviral, lentiviral and adeno-associated viral vectors. In some embodiments, the nucleic acid molecules are introduced into a cell using a retroviral vector following standard procedures well known in the art. Non-viral methods of transfection include any appropriate method that does not use viral DNA or viral particles as a delivery system to introduce the nucleic acid molecule into the cell. Exemplary non-viral transfection methods include, but not limited to, calcium phosphate transfection (or chemical transfection), liposomal transfection (or lipofection), nucleofection, sonoporation, transfection through heat shock (or heat-shock transfection), microinjection, biolistics, magnetifection and electroporation. In some embodiments, cell transfection is carried out by electroporation. In some embodiments, cell transfection is carried out by nucleofection. In some embodiments, cell transfection is carried out by lipofection. The terms “transfection” or “transduction” also refer to introducing ribonucleoprotein complexs (or ribonucleoprotein particles or RNPs) or proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “electroporation,” as used herein, refers to application of an electrical current or electrical field to a cell to facilitate entry of a nucleic acid molecule into the cell. During this process, the electrical current or electrical field applied to the cell increases the permeability of the cell membrane, allowing chemicals, drugs, or nucleic acids (e.g., DNA, RNA, or DNA/RNA hybrid) to be introduced into the cell (also called electrotransfer).

A “ribonucleoprotein complex,” or “ribonucleoprotein particle” or “RNP” as used herein refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid, it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g., dipole-dipole, dipole-induced dipole, London dispersion), ring stacking (pi effects), hydrophobic interactions and the like). In some embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA, thereby forming a ribonucleoprotein complex. Non-limiting examples of ribonucleoproteins include ribosomes, telomerase, RNAseP, hnRNP, CRISPR associated protein 9 (Cas9) and small nuclear RNPs (snRNPs). The ribonucleoprotein may be an enzyme. In some embodiments, the ribonucleoprotein is an endonuclease. In some embodiments, the ribonucleoprotein complex includes an endonuclease and a ribonucleic acid. In some embodiments, the endonuclease is a CRISPR associated protein 9 (Cas9). In some embodiments therefore, the RNPs used in the methods of the disclosure are CRISPR/Cas9 ribonucleoprotein (RNP) complexes.

The term “gene-modifying enzyme” as used herein refers to an enzyme that is capable of modifying a gene by introducing a mutation (e.g., point mutation, frameshift mutation, deletion, or truncation) causing gene inactivation or introducing heterologous nucleotides (e.g., genes) through non-homologous end joining or homologous recombination. Exemplary gene-modifying enzymes, include but not limited to, a Cas protein, a meganuclease, a transcription activator-like effector nucleases (TALEN), a transposon, a zinc-finger nuclease (ZFN), or a recombinase. In some embodiments, the gene-modifying enzyme suitable for the methods disclosed herein is a Cas protein, a meganuclease, a TALEN, a ZFN, or a recombinase. In some embodiments, the gene-modifying enzyme suitable for the methods disclosed herein is a Cas protein. In some preferred embodiments, the gene-modifying enzyme suitable for the methods disclosed herein is a Cas9 protein.

The term “Cas9 protein” refers to the “clustered, regularly interspaced, short palindromic repeats (CRISPR)-associated protein 9.” This term is well known in the art and has been described, e.g. in Makarova et al. (2011) Nat. Rev. Microbiol., 9:467-477, and in Makarova et al. (2011) Biol. Direct., 6:38.

Cas proteins are endonuclease that form part of an adaptive defense mechanism evolved by bacteria and archaea to protect them from invading viruses and plasmids. Cas9 protein or gene information can be obtained from a known database such as the GenBank of NCBI (National Center for Biotechnology Information), but is not limited thereto. Moreover, the Cas9 protein may comprise not only wild-type Cas9, but also deactivated Cas9 (dCas9), or Cas9 variants such as Cas9 nickase. The deactivated Cas9 may be RFN (RNA-guided FokI nuclease) comprising a FokI nuclease domain bound to dCas9, or may be dCas9 to which a transcription activator or repressor domain is bound. In addition, the Cas9 protein is not limited in its origin. For example, the Cas9 protein may be derived from Streptococcus pyogenes, Francisella novicida, Streptococcus thermophiles, Legionella pneumophila, Listeria innocua, or Streptococcus mutans.

Cas9 protein is the major protein element of the CRISPR/Cas9 system, which forms a complex with crRNA (CRISPR RNA) and tracrRNA (trans-activating crRNA) to form activated endonuclease or nickase. “CRISPR system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a nucleic acid sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments, the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is a guanine and N is any naturally occurring nucleic acid. In some embodiments the target DNA comprises any one or combination of NNG, NNA, GAA, NNAGAAW and NGGNG, where G is a guanine, A is adenine, and N is any naturally occurring nucleic acid.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional (bind the Cas protein or functional fragment thereof). In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector.

With at least some of the modification contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5^{mut} with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a type II Cas9 protein. In some embodiments, the chemically synthesized guide sequence is used in conjunction with one or more vectors, wherein each vector comprises a coding sequence that encodes a CRISPR enzyme, such as a type II Cas9 protein. The coding sequence of one element may be located on the same or opposite strand of the coding sequence of a second element, and oriented in the same or opposite direction. In some embodiments, a single promoter drives expression of a transcript encoding a CRISPR enzyme and one or more additional (second, third, fourth, etc.) guide sequences, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, one or more additional guide sequence, tracr mate sequence, and tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid for a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operably linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein.

The CRISPR system suitable for the present disclosure can also comprise a modified CRISPR enzyme (or “Cas protein”) or a nucleotide sequence encoding one or more Cas proteins. Any protein capable of enzymatic activity in cooperation with a guide sequence is a Cas protein. In some embodiments, the disclosure relates to a system comprises a vector comprising a regulatory element operably linked to an enzyme-coding sequence encoding a CRISPR enzyme, such as a Cas protein from the Cas family of enzymes. In some embodiments, the disclosure relates to a system, composition, or pharmaceutical composition comprising any one or plurality of Cas proteins either individually or in combination with one or a plurality of guide sequences. Compositions of one or a plurality of Cas proteins may be administered to a subject with any of the disclosed guide sequences sequentially or contemporaneously. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, type V CRISPR-Cas systems, variants and fragments thereof, or modified versions thereof having at least 70% identity to the sequences of Table X. These enzymes are known, for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme or Cas protein that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase may be used in combination with guide sequence(s), e.g., two guide sequences, which target respectively sense and antisense strands of the DNA target. This combination allows both strands to be nicked and used to induce NHEJ.

As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

TABLE X
Accession numbers of Cas proteins (or those related
with Cas-like function) and nucleic acids encoding the same.
All amino acid and nucleic acid sequences associated with the
Accession Numbers below are incorporated by reference in their entireties.
NC_014644.1; NC_002967.9; NC_007929.1;
NC_000913.3; NC_004547.2; NC_009380.1;
NC_011661.1; NC_010175.1; NC_010175.1;
NC_010175.1; NC_003413.1; NC_000917.1;
NC_002939.5; NC_018227.2; NC_004829.2;
NC_021921.1; NC_014160.1; NC_011766.1;
NC_007681.1; NC_021592.1; NC_021592.1;
NC_021169.1; NC_020517.1; NC_018656.1;
NC_018015.1; NC_018015.1; NC_017946.1;
NC_017576.1; NC_017576.1; NC_015865.1;
NC_015865.1; NC_015680.1; NC_015680.1;
NC_015474.1; NC_015435.1; NC_013790.1;
NC_013790.1; NC_012883.1; NC_012470.1;
NC_016051.1; NC_010610.1; NC_009515.1;
NC_008942.1; NC_007181.1; NC_007181.1;
NC_006624.1; NC_006448.1; NC_002935.2;
NC_002935.2; NC_002950.2; NC_002950.2;
NC_002663.1; NC_002663.1; NC_004557.1;
NC_004557.1; NC_019943.1; NC_019943.1;
NC_019943.1; NC_017459.1; NC_017459.1;
NC_015518.1; NC_015460.1; NC_015416.1;
NC_014933.1; NC_013961.1; NC_013202.1;
NC_013158.1; NC_009464.1; NC_008508.1;
NC_007426.1; NC_000917.1; NC_003901.1;
NC_003901.1; NC_003106.2; NC_009434.1;
NC_005085.1; NC_005085.1; NC_020247.1;
NC_020247.1; NC_020246.1; NC_020246.1;
NC_018224.1; NC_015943.1; NC_011138.3;
NC_009778.1; NC_006834.1; NC_014228.1;
NC_010002.1; NC_013892.1; NC_010296.1;
NC_009615.1; NC_012632.1; NC_012632.1;
NC_012588.1; NC_012588.1; NC_007643.1;
NC_002939.5; NC_011296.1; NC_011296.1;
NC_018609.1; NC_021355.1; NC_021355.1;
NC_020800.1; NC_019942.1; NC_019792.1;
NC_015958.1; NC_015678.1; NC_015636.1;
NC_015562.1; NC_014222.1; NC_014222.1;
NC_014002.1; NC_013887.1; NC_013156.1;
NC_011832.1; NC_009953.1; NC_009635.1;
NC_009634.1; NC_008618.1; NC_007955.1;
NC_007955.1; NC_007955.1; NC_007955.1;
NC_007955.1; NC_007796.1; NC_002754.1;
NC_002754.1; NC_011835.1; NC_013198.1;
NC_000962.3; NC_002163.1; NC_017034.1;
NC_009089.1; NC_008698.1; NC_020419.1;
NC_020419.1; NC_020419.1; NC_015847.1;
NC_014374.1; NC_013520.1; NC_010482.1;
NC_009776.1; NC_009776.1; NC_009033.1;
NC_000916.1; NC_018015.1; NC_015518.1;
NC_014537.1; NC_009440.1; NC_007644.1;
NC_007644.1; NC_022246.1; NC_019943.1;
NC_016023.1; NC_016023.1; NC_015416.1;
NC_013722.1; NC_013722.1; NC_009464.1;
NC_007643.1; NC_007643.1; NC_007643.1;
NC_003106.2; NC_004342.2; NC_018658.1;
NC_017276.1; NC_017275.1; NC_016112.1;
NC_016112.1; NC_003552.1; NC_003197.1;
NC_003198.1; NC_012726.1; NC_012623.1;
NC_015964.1; NC_023069.1; NC_023044.1;
NC_022777.1; NC_022777.1; NC_022777.1;
NC_013769.1; NC_013769.1; NC_011832.1;
NC_011296.1; NC_009712.1; NC_009634.1;
NC_009439.1; NC_009135.1; NC_008599.1;
NC_007796.1; NC_007796.1; NC_007796.1;
NC_007355.1; NC_021082.1; NC_018001.1;
NC_009785.1; NC_022084.1; NC_018092.1;
NC_014804.1; NC_014147.1; NC_009053.1;
NC_000961.1; NC_000961.1; NC_021058.1;
NC_018876.1; NC_018876.1; NC_018081.1;
NC_011567.1; NC_016901.1; NC_014500.1;
NC_013715.1; NC_019977.1; NC_019042.1;
NC_017274.1; NC_015954.1; NC_015676.1;
NC_015320.1; NC_014122.1; NC_014122.1;
NC_013407.1; NC_014961.1; NC_013926.1;
NC_013926.1; NC_021353.1; NC_008818.1;
NC_021058.1; NC_015151.1; NC_013849.1;
NC_009051.1; NC_018876.1; NC_018876.1;
NC_014507.1; NC_015574.1; NC_014500.1;
NC_012622.1; NC_012589.1; NC_009515.1;
NC_017275.1; NC_000913.3; NC_017527.1;
NC_018227.2; NC_007355.1; NC_014106.1;
NC_010610.1; NC_008054.1; NC_007164.1;
NC_015760.1; NC_009953.1; NC_010572.1;
NC_009613.3; NC_014334.1; NC_008526.1;
NC_026150.1; NC_015776.1; NC_007116.6;
NC_012779.2; NC_003901.1; NC_020892.1;
NC_011832.1; NC_003143.1; NC_003143.1;
NC_008800.1; NC_011308.1; NC_008942.1;
NC_007297.1; NC_005877.1; NC_005877.1;
NC_002689.2; NC_006085.1; NC_004116.1;
NC_010397.1; NC_009917.1; NC_012490.1;
NC_006067.1; NW_004197518.1; NC_022777.1;
NC_019042.1; NC_004547.2; NC_002695.1;
NC_017634.1; NC_003143.1; NC_002737.2;
NC_002737.2; NC_000918.1; NC_020913.1;
NC_006448.1; NC_022093.1; NC_022093.1;
NC_015680.1; NC_007297.1; NC_004350.2;
NC_004350.2; NC_004350.2; NC_004350.2;
NC_003454.1; NC_000853.1; NC_018876.1;
NC_009440.1; NC_009009.1; NC_009009.1;
NC_002932.3; NC_002932.3; NC_026150.1;
NC_003552.1; NC_025263.1; NC_016112.1;
NC_011098.1; NC_007643.1; NC_007643.1;
NC_007643.1; NC_006347.1; NC_005140.1;
NC_004342.2; NC_002945.3; NW_007382731.1;
NW_007381138.1; NC_024320.1;
NW_005756335.1; NW_003384463.1; NC_019977.1;
NC_011296.1; NC_007929.1;
NC_000913.3; NC_003413.1; NC_002754.1;
NC_010175.1; NC_010175.1; NC_010175.1;
NC_011661.1; NC_014537.1; NC_012470.1;
NC_004829.2; NC_015516.1; NC_014374.1;
NC_009033.1; NC_007681.1; NC_002689.2;
NC_006085.1; NC_021592.1; NC_021592.1;
NC_021169.1; NC_020517.1; NC_018015.1;
NC_018015.1; NC_018015.1; NC_017946.1;
NC_017946.1; NC_017576.1; NC_017576.1;
NC_015865.1; NC_015865.1; NC_015847.1;
NC_015680.1; NC_015680.1; NC_015474.1;
NC_015435.1; NC_014106.1; NC_013790.1;
NC_012883.1; NC_012804.1; NC_016051.1;
NC_011529.1; NC_010482.1; NC_009515.1;
NC_009440.1; NC_008942.1; NC_008054.1;
NC_007181.1; NC_006624.1; NC_006448.1;
NC_006448.1; NC_002935.2; NC_002935.2;
NC_002950.2; NC_002663.1; NC_019943.1;
NC_019943.1; NC_017459.1; NC_017459.1;
NC_016023.1; NC_015518.1; NC_015460.1;
NC_015460.1; NC_015416.1; NC_014933.1;
NC_013202.1; NC_013158.1; NC_009464.1;
NC_008508.1; NC_003901.1; NC_009434.1;
NC_005085.1; NC_020247.1; NC_020246.1;
NC_018224.1; NC_015943.1; NC_009380.1;
NC_006834.1; NC_003552.1; NC_017276.1;
NC_017275.1; NC_010296.1; NC_009615.1;
NC_012632.1; NC_012632.1; NC_012623.1;
NC_012588.1; NC_012588.1; NC_007181.1;
NC_002939.5; NC_020247.1; NC_020246.1;
NC_011296.1; NC_011296.1; NC_011296.1;
NC_018609.1; NC_015964.1; NC_021355.1;
NC_020800.1; NC_019942.1; NC_019792.1;
NC_015958.1; NC_015760.1; NC_015678.1;
NC_015636.1; NC_015562.1; NC_014222.1;
NC_014222.1; NC_013887.1; NC_013769.1;
NC_013156.1; NC_009953.1; NC_009635.1;
NC_009634.1; NC_009135.1; NC_008618.1;
NC_008599.1; NC_007955.1; NC_007796.1;
NC_007355.1; NC_002754.1; NC_010572.1;
NC_015151.1; NC_000962.3; NC_021921.1;
NC_002163.1; NC_017034.1; NC_009089.1;
NC_008698.1; NC_020419.1; NC_020419.1;
NC_020419.1; NC_014160.1; NC_011766.1;
NC_007681.1; NC_000916.1; NC_017527.1;
NC_013790.1; NC_013790.1; NC_000917.1;
NC_000917.1; NC_004557.1; NC_004557.1;
NC_022246.1; NC_017384.1; NC_013722.1;
NC_007643.1; NC_007643.1; NC_007643.1;
NC_007643.1; NC_007643.1; NC_002967.9;
NC_004342.2; NC_016112.1; NC_016112.1;
NC_005140.1; NC_005140.1; NC_012726.1;
NC_023069.1; NC_023044.1; NC_022777.1;
NC_022777.1; NC_011296.1; NC_021355.1;
NC_009634.1; NC_007796.1; NC_007355.1;
NC_021082.1; NC_013926.1; NC_020913.1;
NC_014961.1; NC_014658.1; NC_013198.1;
NC_005877.1; NC_009785.1; NC_022084.1;
NC_018092.1; NC_014804.1; NC_000961.1;
NC_021058.1; NC_018081.1; NC_013849.1;
NC_011567.1; NC_015574.1; NC_014500.1;
NC_012622.1; NC_012589.1; NC_012589.1;
NC_019977.1; NC_019042.1; NC_017274.1;
NC_017274.1; NC_015954.1; NC_015676.1;
NC_015320.1; NC_014122.1; NC_014122.1;
NC_013407.1; NC_011835.1; NC_021353.1;
NC_018001.1; NC_008818.1; NC_000961.1;
NC_015931.1; NC_019042.1; NC_013961.1;
NC_011138.3; NC_009778.1; NC_014228.1;
NC_013892.1; NC_011832.1; NC_009439.1;
NC_007955.1; NC_007796.1; NC_013520.1;
NC_016070.1; NC_007426.1; NC_003106.2;
NC_003106.2; NC_018227.2; NC_000913.3;
NC_005085.1; NC_009613.3; NC_014334.1;
NW_006726754.1; NC_002663.1; NC_003143.1;
NC_003076.8; NC_015666.1; NC_014644.1;
NC_004116.1; NC_003454.1; NC_011567.1;
NC_024905.1; NC_003295.1; NC_008526.1;
NC_012871.1; NC_012871.1; NC_010682.1;
NC_002737.2; NC_002737.2; NC_017954.1;
NC_009515.1; NC_007297.1; NC_007297.1;
NC_004350.2; NC_004350.2; NC_000853.1;
NC_009009.1; NC_007644.1; NC_007644.1;
NC_002967.9; NC_002932.3; NC_002932.3;
NC_007643.1; NC_007606.1; NC_006347.1;
NC_002945.3; NW_006804726.1;
NW_006383769.1; NC_013769.1; NC_014644.1;
NC_000913.3; NC_019943.1; NC_019943.1;
NC_011661.1; NC_010175.1; NC_002950.2;
NC_004547.2; NC_013887.1; NC_013156.1;
NC_007426.1; NC_002939.5; NC_021169.1;
NC_020517.1; NC_018015.1; NC_017946.1;
NC_015865.1; NC_015680.1; NC_009515.1;
NC_004557.1; NC_005085.1; NC_006834.1;
NC_011296.1; NC_010175.1; NC_020800.1;
NC_015958.1; NC_009635.1; NC_008618.1;
NC_007355.1; NC_009089.1; NC_020419.1;
NC_021592.1; NC_021592.1; NC_015847.1;
NC_013790.1; NC_016051.1; NC_007644.1;
NC_007644.1; NC_017459.1; NC_015416.1;
NC_013722.1; NC_007643.1; NC_007643.1;
NC_007643.1; NC_009434.1; NC_005085.1;
NC_003552.1; NC_014318.1; NC_021355.1;
NC_014222.1; NC_014222.1; NC_011832.1;
NC_009634.1; NC_009135.1; NC_021082.1;
NC_000961.1; NC_015574.1; NC_014228.1;
NC_014122.1; NC_009439.1; NC_017459.1;
NC_015460.1; NC_011138.3; NC_009380.1;
NC_017275.1; NC_013892.1; NC_021353.1;
NC_015676.1; NC_011296.1; NC_007955.1;
NC_009953.1; NC_009953.1; NC_021921.1;
NC_014160.1; NC_010482.1; NC_009776.1;
NC_009033.1; NC_016070.1; NC_016070.1;
NC_015435.1; NC_009440.1; NC_017384.1;
NC_013722.1; NC_016112.1; NC_012726.1;
NC_022777.1; NC_008698.1; NC_008599.1;
NC_007955.1; NC_007355.1; NC_014147.1;
NC_021058.1; NC_021058.1; NC_016901.1;
NC_014500.1; NC_014500.1; NC_014961.1;
NC_018001.1; NC_015931.1; NC_015151.1;
NC_013849.1; NC_013715.1; NC_011766.1;
NC_018001.1; NC_014644.1; NC_017034.1;
NC_009033.1; NC_002754.1; NC_009089.1;
NC_002939.5; NC_014106.1; NC_010610.1;
NC_008054.1; NC_003413.1; NC_009464.1;
NC_008526.1; NC_015474.1; NC_012804.1;
NC_015518.1; NC_017276.1; NC_017275.1;
NC_012632.1; NC_012623.1; NC_012588.1;
NC_015636.1; NC_015562.1; NC_013769.1;
NC_002754.1; NC_017634.1; NC_014160.1;
NC_011766.1; NC_016070.1; NC_015435.1;
NC_009440.1; NC_009440.1; NC_012726.1;
NC_012632.1; NC_012588.1; NC_013887.1;
NC_013156.1; NC_011296.1; NC_002754.1;
NC_011835.1; NC_018092.1; NC_021058.1;
NC_012622.1; NC_012589.1; NC_015954.1;
NC_013407.1; NC_018001.1; NC_013849.1;
NC_017274.1; NC_000913.3; NC_003413.1;
NC_018092.1; NC_000961.1; NC_000918.1;
NC_007796.1; NC_000868.1; NC_022084.1;
NC_018015.1; NC_015865.1; NC_015680.1;
NC_015474.1; NC_014804.1; NC_012470.1;
NC_006624.1; NC_002663.1; NC_016023.1;
NC_013202.1; NC_013158.1; NC_008508.1;
NC_000917.1; NC_015943.1; NC_019792.1;
NC_019042.1; NC_015760.1; NC_015678.1;
NC_014122.1; NC_004119.1; NC_007681.1;
NC_007681.1; NC_007297.1; NC_002935.2;
NC_002932.3; NC_003454.1; NC_014933.1;
NC_011567.1; NC_004342.2; NC_016112.1;
NC_003197.1; NC_022777.1; NC_015320.1;
NC_002695.1; NC_003143.1; NC_002737.2;
NC_012883.1; NC_010610.1; NC_000916.1;
NC_004350.2; NC_000853.1; NC_000917.1;
NC_006347.1; NC_018658.1; NC_015870.2;
NC_011751.1; NC_013961.1; NC_009778.1;
NC_020990.1; NC_016112.1; NC_000868.1;
NC_003413.1; NC_022084.1; NC_018092.1;
NC_017946.1; NC_015680.1; NC_015680.1;
NC_015474.1; NC_014106.1; NC_012804.1;
NC_009053.1; NC_008054.1; NC_006624.1;
NC_000961.1; NC_021058.1; NC_015518.1;
NC_018224.1; NC_017276.1; NC_017276.1;
NC_017275.1; NC_017275.1; NC_010296.1;
NC_010296.1; NC_009615.1; NC_012632.1;
NC_012632.1; NC_012632.1; NC_012623.1;
NC_012623.1; NC_012622.1; NC_012622.1;
NC_012589.1; NC_012589.1; NC_012588.1;
NC_012588.1; NC_012588.1; NC_020892.1;
NC_019792.1; NC_017970.1; NC_017274.1;
NC_017274.1; NC_016159.1; NC_013887.1;
NC_013769.1; NC_013156.1; NC_002754.1;
NC_002754.1; NC_002754.1; NC_003687.1;
NC_006814.3; NC_006814.3; NC_014418.1;
NC_010152.1; NC_017946.1; NC_017954.1;
NC_009776.1; NC_008818.1; NC_008818.1;
NC_000961.1; NC_000918.1; NC_015931.1;
NC_015931.1; NC_014537.1; NC_007181.1;
NC_006624.1; NC_003106.2; NC_004342.2;
NC_020247.1; NC_020246.1; NC_018472.1;
NC_012623.1; NC_012589.1; NC_006045.2;
NC_023069.1; NC_022777.1; NC_019942.1;
NC_017274.1; NC_013769.1; NC_009953.1;
NC_008698.1; NC_007493.2; NC_002754.1;
NC_002754.1; NC_005125.1; NC_021347.1;
NC_022093.1; NC_022093.1; NC_015931.1;
NC_007164.1; NC_015416.1; NC_015151.1;
NC_000917.1; NC_003106.2; NC_002932.3;
NC_014500.1; NC_004337.2; NC_007087.3;
NC_012726.1; NC_024314.1; NW_003120284.1;
NW_003120529.1; NW_003126883.1;
NW_003384275.1; NC_023069.1; NC_016567.1;
NC_009954.1; NC_000913.3; NC_000913.3;
NC_000913.3; NC_000913.3; NC_027204.1;
NC_002754.1; NC_010175.1; NC_016070.1;
NC_000868.1; NC_003413.1; NC_017527.1;
NC_002939.5; NC_018227.2; NC_007355.1;
NC_014205.1; NC_014160.1; NC_009033.1;
NC_007681.1; NC_020517.1; NC_018015.1;
NC_016070.1; NC_015865.1; NC_015847.1;
NC_015680.1; NC_015474.1; NC_015315.1;
NC_013790.1; NC_012883.1; NC_012470.1;
NC_016051.1; NC_011529.1; NC_010610.1;
NC_009515.1; NC_009440.1; NC_007181.1;
NC_006624.1; NC_019943.1; NC_019943.1;
NC_017459.1; NC_015518.1; NC_014933.1;
NC_007426.1; NC_003901.1; NC_003106.2;
NC_003106.2; NC_009434.1; NC_005085.1;
NC_020247.1; NC_020246.1; NC_006834.1;
NC_017276.1; NC_017276.1; NC_017276.1;
NC_017275.1; NC_017275.1; NC_010296.1;
NC_009615.1; NC_012632.1; NC_012632.1;
NC_012623.1; NC_012623.1; NC_012588.1;
NC_012588.1; NC_007643.1; NC_021355.1;
NC_020800.1; NC_015958.1; NC_015678.1;
NC_014222.1; NC_013769.1; NC_013769.1;
NC_009635.1; NC_009634.1; NC_009135.1;
NC_008618.1; NC_007796.1; NC_002754.1;
NC_002754.1; NC_017034.1; NC_019413.1;
NC_020419.1; NC_017946.1; NC_014374.1;
NC_014374.1; NC_011766.1; NC_011766.1;
NC_010482.1; NC_009776.1; NC_009776.1;
NC_009033.1; NC_008942.1; NC_021169.1;
NC_015518.1; NC_014537.1; NC_012804.1;
NC_007644.1; NC_015416.1; NC_013722.1;
NC_007643.1; NC_016112.1; NC_016112.1;
NC_012726.1; NC_012726.1; NC_023069.1;
NC_023069.1; NC_021355.1; NC_019942.1;
NC_014002.1; NC_021082.1; NC_005830.1;
NC_018001.1; NC_017954.1; NC_014961.1;
NC_022084.1; NC_018092.1; NC_014804.1;
NC_021058.1; NC_021058.1; NC_021058.1;
NC_015574.1; NC_015216.1; NC_014500.1;
NC_012622.1; NC_012622.1; NC_012589.1;
NC_012589.1; NC_012589.1; NC_005265.1;
NC_011661.1; NC_019977.1; NC_017274.1;
NC_017274.1; NC_017274.1; NC_015954.1;
NC_008701.1; NC_009965.1; NC_014961.1;
NC_013926.1; NC_018001.1; NC_013741.1;
NC_013741.1; NC_008818.1; NC_000961.1;
NC_015931.1; NC_015931.1; NC_015151.1;
NC_015151.1; NC_013849.1; NC_015320.1;
NC_014122.1; NC_000913.3; NC_011217.1;
NC_003413.1; NC_005360.1; NC_005361.1;
NC_009986.1; NC_021921.1; NC_007681.1;
NC_021169.1; NC_017946.1; NC_015865.1;
NC_015680.1; NC_015474.1; NC_012804.1;
NC_002950.2; NC_004557.1; NC_017459.1;
NC_016023.1; NC_013202.1; NC_013158.1;
NC_018224.1; NC_015943.1; NC_003552.1;
NC_012632.1; NC_012588.1; NC_011296.1;
NC_011296.1; NC_021355.1; NC_019942.1;
NC_019792.1; NC_015958.1; NC_007955.1;
NC_009089.1; NC_018015.1; NC_018015.1;
NC_007644.1; NC_004557.1; NC_022777.1;
NC_022777.1; NC_015562.1; NC_008599.1;
NC_007355.1; NC_018092.1; NC_000961.1;
NC_011567.1; NC_013926.1; NC_021353.1;
NC_022084.1; NC_015574.1; NC_003106.2;
NC_015320.1; NC_016070.1; NC_013588.1;
NC_011835.1; NC_011766.1; NC_009776.1;
NC_009033.1; NC_010482.1; NC_009440.1;
NC_015518.1; NC_003106.2; NC_017276.1;
NC_017275.1; NC_012632.1; NC_012623.1;
NC_012588.1; NC_013769.1; NC_002754.1;
NC_017034.1; NC_014374.1; NC_015416.1;
NC_003106.2; NC_012726.1; NC_013715.1;
NC_025439.1; NC_017954.1; NC_014961.1;
NC_013849.1; NC_012622.1; NC_012589.1;
NC_017274.1; NC_018001.1; NC_015931.1;
NC_009380.1; NC_000010.11; NC_006477.3;
AC_000185.1; NC_013907.1; NC_025687.1;
NC_012021.3; NC_023046.1; NC_023650.1;
NW_006159565.1; NC_018160.1; NC_027878.1;
NC_024803.1; NW_007540324.1;
NC_020497.1; NC_026657.1; NC_003280.10;
NC_004354.4; NC_001140.6;
NC_003070.9; NT_078265.2; NW_006890056.1;
NW_003613826.1; NC_026506.1;
NC_023616.1; NC_025973.2; NC_024333.1;
NW_006717075.1; NW_006438113.1;
NW_006399752.1; NW_005785394.1; NC_019873.1;
NC_019482.1; NC_018902.1; NC_022199.1;
NC_008801.1; NC_026145.1; NC_015446.2;
NC_026940.1; NC_025695.1; NC_025207.1;
NW_006749282.1; NC_015438.2; NC_016093.1;
NC_007418.2; NC_000076.6; NC_003423.3;
NC_003423.3; NC_001134.8; NC_015474.1;
NC_005104.4; NC_007126.6; NC_007124.6;
NC_006093.3; NC_024464.1; NC_007124.6;
NC_008397.2; NC_010572.1; NC_010572.1;
NC_017301.1; NC_017941.2; NC_016407.2;
NC_016410.2; NC_012026.1; NC_021921.1;
NC_018092.1; NC_017946.1; NC_015865.1;
NC_015680.1; NC_015435.1; NC_012804.1;
NC_016051.1; NC_011529.1; NC_009515.1;
NC_009515.1; NC_009440.1; NC_007164.1;
NC_006624.1; NC_002935.2; NC_000916.1;
NC_017459.1; NC_015151.1; NC_014933.1;
NC_014729.1; NC_014537.1; NC_013209.1;
NC_013202.1; NC_013158.1; NC_011567.1;
NC_010364.1; NC_008212.1; NC_007426.1;
NC_002607.1; NC_003366.1; NC_023013.1;
NC_018224.1; NC_016946.1; NC_016946.1;
NC_015948.1; NC_015666.1; NC_014297.1;
NC_013967.1; NC_013922.1; NC_013743.1;
NC_009783.1; NC_009380.1; NC_005786.2;
NC_005785.6; NC_006396.1; NC_002944.2;
NC_010002.1; NC_020990.1; NC_009615.1;
NC_009614.1; NC_008313.1; NC_013929.1;
NC_017765.1; NC_017844.1; NC_014323.1;
NC_007973.1; NC_019962.1; NC_019792.1;
NC_019042.1; NC_016114.1; NC_016114.1;
NC_015954.1; NC_011753.2; NC_010505.1;
NC_009953.1; NC_009953.1; NC_008268.1;
NC_007866.1; NC_008596.1; NC_008596.1;
NC_000868.1; NC_004668.1; NC_009760.1;
NC_006820.1; NC_003888.3; NC_025463.1;
NC_021921.1; NC_021921.1; NC_006883.2;
NC_017034.1; NC_014423.1; NC_017941.2;
NC_004603.1; NC_007088.5; NC_008367.1;
NC_021776.1; NT_078267.5; NC_002656.1;
NC_022774.1; NC_011091.1; NC_005881.2;
NC_011183.1; NC_015937.1; NC_008584.1;
NC_024122.1; NC_022768.1; NC_022772.1;
NC_013085.1; NC_010154.1; NC_010152.1;
NC_010155.1; NC_009804.1; NC_009803.1;
NC_005342.2; NC_004333.2; NC_023735.1;
NC_023694.1; NC_027364.1; NC_019526.1;
NC_023607.1; NC_021353.1; NC_021592.1;
NC_012039.1; NC_008942.1; NC_002936.3;
NC_005877.1; NC_021169.1; NC_021058.1;
NC_020517.1; NC_020388.1; NC_020388.1;
NC_018656.1; NC_015435.1; NC_014804.1;
NC_013790.1; NC_013790.1; NC_009440.1;
NC_009051.1; NC_007929.1; NC_007929.1;
NC_005042.1; NC_003454.1; NC_003238.2;
NC_021313.1; NC_019943.1; NC_019943.1;
NC_017459.1; NC_017384.1; NC_015288.1;
NC_015287.1; NC_015284.1; NC_015281.1;
NC_015280.1; NC_014334.1; NC_014297.1;
NC_013967.1; NC_013202.1; NC_011129.1;
NC_007426.1; NC_007426.1; NC_003901.1;
NC_003901.1; NC_004342.2; NC_014622.2;
NC_023731.1; NC_023729.1; NC_023716.1;
NC_017275.1; NC_015574.1; NC_015216.1;
NC_015216.1; NC_013922.1; NC_013922.1;
NC_013743.1; NC_012966.1; NC_012966.1;
NC_011913.1; NC_010397.1; NC_010296.1;
NC_009380.1; NC_006396.1; NC_006347.1;
NC_002944.2; NC_003552.1; NC_004663.1;
NW_006890135.1; NC_026750.1; NC_021285.1;
NC_017765.1; NC_014942.1; NC_014435.1;
NC_014318.1; NC_008526.1; NC_007197.1;
NC_006687.1; NC_022543.1; NC_022528.1;
NC_022528.1; NC_012623.1; NC_012622.1;
NC_012589.1; NC_023617.1; NC_006049.2;
NC_006048.2; NC_013486.1; NC_006071.1;
NC_006038.1; NC_006037.1; NC_012878.1;
NC_012875.1; NC_008480.2; NC_011089.1;
NW_007907148.1; NW_008246060.1;
NW_007726404.1; NW_007677829.1;
NW_007620716.1; NC_024318.1; NC_024311.1;
NW_007578198.1; NW_007370681.1;
NW_007249782.1; NC_023643.1; NW_006921657.1;
NW_006799972.1; NW_006804151.1;
NW_006784004.1; NW_006532274.1;
NW_006725466.1; NW_006712311.1;
NW_006610005.1; NW_006501061.1; NW_006399824.1;
NW_006408538.1; NW_006288853.1;
NW_006383157.1; NW_006224511.1;
NW_005882862.1; NW_006210709.1; NW_006159730.1;
NW_005859001.1; NW_005855688.1; NW_005871110.1;
NW_005843034.1; NW_005819063.1;
NW_005815230.1; NW_005395486.1;
NW_005369027.1; NC_022320.1; NC_022280.1;
NC_022028.1; NW_007359849.1; NW_007359875.1;
NC_021678.1; NW_004454176.1;
NW_004205048.1; NC_019861.1; NC_018434.1;
NC_018733.2; NC_018893.1; NC_016133.1;
NC_016135.1; NC_016098.1; NC_010459.4;
NC_022211.1; NC_015018.2; NC_013686.1;
NC_011470.1; NC_009144.2; NC_007796.1;
NC_018609.1; NC_027340.1; NC_027218.1;
NC_027132.1; NC_026928.1; NC_026583.1;
NC_026440.1; NC_025422.1; NC_025447.1;
NC_023862.1; NC_023553.1; NC_023587.1;
NC_023564.1; NC_022973.1; NC_022984.1;
NC_022086.1; NC_021307.1; NC_021321.1;
NC_021335.1; NC_021320.1; NC_020159.1;
NC_019974.1; NC_019974.1; NC_019974.1;
NC_019974.1; NC_019962.1; NC_019962.1;
NC_019942.1; NC_018283.1; NC_018227.2;
NC_016653.1; NC_016570.1; NC_013600.1;
NC_015954.1; NC_015958.1; NC_015676.1;
NC_015274.1; NC_015296.1; NC_014371.1;
NC_014253.1; NC_013769.1; NC_013084.1;
NC_013081.1; NC_013079.1; NC_012865.1;
NC_012029.1; NC_011023.1; NC_011019.1;
NC_010537.1; NC_009954.1; NC_007796.1;
NC_007493.2; NC_007355.1; NC_002946.2;
NC_002946.2; NC_002519.1; NC_006089.3;
NC_006089.3; NC_003214.2; NC_010356.1;
NC_004463.1; NC_007797.1; NC_002737.2;
NC_025375.1; NC_005125.1; NC_027120.1;
NC_018280.1; NC_016566.1; NC_007077.3;
NC_019419.2; NC_012223.2; NC_021783.1;
NC_021342.2; NC_021315.1; NC_020204.1;
NC_025443.1; NC_023549.1; NC_019924.1;
NC_019550.1; NC_019549.1; NC_014457.1;
NC_023566.1; NC_004927.1; NC_003345.1;
NC_014322.1; NC_027119.1; NC_019452.1;
NC_010153.1; NC_021780.1; NC_021775.1;
NC_020419.1; NC_020419.1; NC_013926.1;
NC_005364.2; NC_017954.1; NC_012039.1;
NC_000918.1; NC_013790.1; NC_022093.1;
NC_022093.1; NC_022093.1; NC_022093.1;
NC_021592.1; NC_021169.1; NC_018015.1;
NC_015931.1; NC_015435.1; NC_014408.1;
NC_013790.1; NC_012883.1; NC_008818.1;
NC_008818.1; NC_008818.1; NC_008818.1;
NC_007297.1; NC_006570.2; NC_000916.1;
NC_004350.2; NC_003454.1; NC_000853.1;
NC_018876.1; NC_017527.1; NC_021313.1;
NC_020388.1; NC_015518.1; NC_015416.1;
NC_015416.1; NC_015151.1; NC_015151.1;
NC_014537.1; NC_014537.1; NC_014537.1;
NC_014537.1; NC_014507.1; NC_013849.1;
NC_013849.1; NC_009464.1; NC_000917.1;
NC_000917.1; NC_000917.1; NC_000917.1;
NC_002932.3; NC_026153.1; NC_020177.2;
NC_027207.1; NC_025260.1; NC_018224.1;
NC_016449.1; NC_016453.1; NC_015574.1;
NC_007643.1; NC_007643.1; NC_026756.1;
NC_026492.1; NC_026490.1; NC_021366.1;
NC_014946.1; NC_014950.1; NC_009667.1;
NC_009048.1; NC_006682.1; NW_001820527.3;
NC_018213.1; NC_009364.1; NC_026477.1;
NC_017844.1; NC_016494.1; NC_016492.1;
NC_016457.1; NC_016475.1; NC_016507.1;
NC_016502.1; NC_012726.1; NC_011672.1;
NC_011687.1; NC_006036.2; NC_006032.2;
NC_026933.1; NC_026006.1; NC_026003.1;
NC_015018.2; NC_025820.1; NC_025701.1;
NC_024796.1; NC_024460.1; NW_007378989.1;
NW_007377463.1; NW_007546349.1;
NW_007546273.1; NC_024245.1; NC_024241.1;
NC_024130.1; NC_024126.1;
NW_006921822.1; NW_006534794.1; NW_006749448.1;
NW_006601119.1; NW_003140815.1;
NW_006272001.1; NW_006280498.1;
NC_023166.1; NC_023167.1; NC_023200.1;
NC_023183.1; NW_006263308.1;
NC_016107.1; NC_023053.1; NW_006238943.1;
NW_006239104.1; NW_006235748.1;
NW_006190770.1; NW_006188714.1;
NW_005871025.1; NW_005872046.1;
NW_005841850.1; NW_005372290.1;
NC_010456.4; NC_021165.1; NC_021163.1;
NC_020492.1; NC_020496.1; NC_015448.2;
NC_026660.1; NW_003803976.1; NC_019833.1;
NC_012601.1; NW_003139842.1;
NC_012012.3; NW_003384299.1;
NW_001760161.1; NC_008802.1; NC_027383.1;
NC_023044.1; NC_026591.1; NC_025446.1;
NC_025442.1; NC_025428.1; NC_024783.1;
NC_023856.1; NC_023755.1; NC_023753.1;
NC_023751.1; NC_023749.1; NC_023749.1;
NC_020853.1; NC_020851.1; NC_020845.1;
NC_020844.1; NC_020837.1; NC_020082.1;
NC_019977.1; NC_019942.1; NC_019791.1;
NC_019724.1; NC_019530.1; NC_019524.1;
NC_019519.1; NC_019518.1; NC_019457.1;
NC_019417.1; NC_019401.1; NC_018396.1;
NC_016520.1; NC_016531.1; NC_016073.1;
NC_015676.1; NC_015569.1; NC_015466.1;
NC_015320.1; NC_012995.1; NC_012992.1;
NC_012867.1; NC_011832.1; NC_010175.1;
NC_009954.1; NC_009712.1; NC_007710.1;
NC_009189.1; NC_009181.1; NC_008698.1;
NC_008698.1; NC_007955.1; NC_007796.1;
NC_012589.1; NC_012588.1; NC_004086.1;
NC_004342.2; NC_022096.1; NC_014644.1;
NC_011661.1; NC_010175.1; NC_002754.1;
NC_002754.1; NC_003413.1; NC_021921.1;
NC_002754.1; NC_018015.1; NC_018015.1;
NC_015865.1; NC_015847.1; NC_015680.1;
NC_015680.1; NC_015680.1; NC_015474.1;
NC_015474.1; NC_015435.1; NC_012804.1;
NC_012470.1; NC_009515.1; NC_009440.1;
NC_006624.1; NC_006624.1; NC_004557.1;
NC_004557.1; NC_016023.1; NC_015518.1;
NC_013202.1; NC_013158.1; NC_003106.2;
NC_003106.2; NC_015943.1; NC_011138.3;
NC_009778.1; NC_009380.1; NC_003552.1;
NC_014228.1; NC_017276.1; NC_017275.1;
NC_013892.1; NC_012632.1; NC_012632.1;
NC_012623.1; NC_012588.1; NC_012588.1;
NC_011296.1; NC_011296.1; NC_019792.1;
NC_015678.1; NC_014222.1; NC_013887.1;
NC_013887.1; NC_013769.1; NC_013156.1;
NC_013156.1; NC_011832.1; NC_009953.1;
NC_009635.1; NC_009634.1; NC_009439.1;
NC_008618.1; NC_007955.1; NC_007355.1;
NC_010572.1; NC_016070.1; NC_000868.1;
NC_009089.1; NC_020419.1; NC_021592.1;
NC_017946.1; NC_017946.1; NC_011766.1;
NC_010482.1; NC_009776.1; NC_009033.1;
NC_021169.1; NC_014537.1; NC_013790.1;
NC_016051.1; NC_000917.1; NC_003454.1;
NC_018224.1; NC_017459.1; NC_015416.1;
NC_014933.1; NC_016112.1; NC_003198.1;
NC_012726.1; NC_014222.1; NC_009135.1;
NC_023069.1; NC_023044.1; NC_022777.1;
NC_021355.1; NC_019942.1; NC_015958.1;
NC_015760.1; NC_009953.1; NC_008599.1;
NC_022084.1; NC_018092.1; NC_018092.1;
NC_000961.1; NC_021058.1; NC_011567.1;
NC_015574.1; NC_012622.1; NC_012589.1;
NC_002950.2; NC_000913.3; NC_017274.1;
NC_015676.1; NC_015320.1; NC_014122.1;
NC_014122.1; NC_017301.1; NC_021353.1;
NC_018001.1; NC_018001.1; NC_000961.1;
NC_000961.1; NC_015931.1; NC_015931.1;
NC_013849.1; NC_002950.2; NC_002663.1;
NC_015460.1; NC_005085.1; NC_010002.1;
NC_013722.1; NC_003106.2; NC_007796.1;
NC_014147.1; NC_009053.1; NC_016901.1;
NC_014500.1; NC_003413.1; NC_016070.1;
NC_022777.1; NC_015516.1; NC_020517.1;
NC_014106.1; NC_010610.1; NC_008942.1;
NC_008054.1; NC_007164.1; NC_002935.2;
NC_013961.1; NC_009434.1; NC_006834.1;
NC_020990.1; NC_009615.1; NC_020800.1;
NC_021082.1; NC_009089.1; NC_014160.1;
NC_007644.1; NC_019943.1; NC_017384.1;
NC_013722.1; NC_007643.1; NC_007643.1;
NC_007643.1; NC_002939.5; NC_004342.2;
NC_005085.1; NC_008526.1; NC_014500.1;
NC_009515.1; NC_003280.10; NC_008508.1;
NC_008508.1; NC_004342.2; NC_010296.1;
NC_003197.1; NC_007090.3; NW_006623966.1;
NC_015020.2; NC_019042.1; NC_011273.1;
NC_002695.1; NC_002737.2; NC_007581.1;
NC_017954.1; NC_014961.1; NC_014658.1;
NC_007681.1; NC_007681.1; NC_000918.1;
NC_000916.1; NC_022093.1; NC_021592.1;
NC_015865.1; NC_014804.1; NC_012883.1;
NC_010610.1; NC_008818.1; NC_007297.1;
NC_004350.2; NC_000853.1; NC_015151.1;
NC_007644.1; NC_000917.1; NC_004113.1;
NC_002932.3; NC_002932.3; NC_022239.1;
NC_016112.1; NC_016112.1; NC_003901.1;
NC_018658.1; NC_014431.1; NC_014318.1;
NC_011751.1; NC_009359.1; NC_026939.1;
NC_024803.1; NC_024313.1; NW_005890115.1;
NC_026659.1; NC_023752.1; NC_009954.1;
NC_007955.1; NC_007796.1; NC_025443.1;
NC_025687.1; NC_009698.1; NC_022777.1;
NC_002754.1; NC_000961.1; NC_011661.1;
NC_000868.1; NC_000868.1; NC_003413.1;
NC_003413.1; NC_021921.1; NC_000916.1;
NC_021169.1; NC_018015.1; NC_018015.1;
NC_018015.1; NC_017946.1; NC_017946.1;
NC_015865.1; NC_015865.1; NC_015865.1;
NC_015680.1; NC_015680.1; NC_015680.1;
NC_015474.1; NC_015474.1; NC_012883.1;
NC_012804.1; NC_012804.1; NC_016051.1;
NC_016051.1; NC_011529.1; NC_011529.1;
NC_007181.1; NC_006624.1; NC_006624.1;
NC_006624.1; NC_006448.1; NC_004557.1;
NC_004557.1; NC_017459.1; NC_015518.1;
NC_015518.1; NC_015460.1; NC_013202.1;
NC_013158.1; NC_000917.1; NC_020247.1;
NC_020246.1; NC_003552.1; NC_017275.1;
NC_010296.1; NC_012632.1; NC_012632.1;
NC_012588.1; NC_012588.1; NC_011296.1;
NC_011296.1; NC_021355.1; NC_019792.1;
NC_015958.1; NC_015636.1; NC_015562.1;
NC_013887.1; NC_013156.1; NC_007955.1;
NC_007355.1; NC_002754.1; NC_002754.1;
NC_009778.1; NC_000962.3; NC_009089.1;
NC_021592.1; NC_017946.1; NC_015680.1;
NC_018015.1; NC_014537.1; NC_014537.1;
NC_012883.1; NC_012804.1; NC_018224.1;
NC_017459.1; NC_016023.1; NC_015943.1;
NC_023069.1; NC_023044.1; NC_019942.1;
NC_014222.1; NC_008599.1; NC_002754.1;
NC_022084.1; NC_022084.1; NC_022084.1;
NC_018092.1; NC_014804.1; NC_014804.1;
NC_021058.1; NC_017274.1; NC_015320.1;
NC_014122.1; NC_013407.1; NC_014658.1;
NC_000961.1; NC_000961.1; NC_018092.1;
NC_015151.1; NC_013849.1; NC_011567.1;
NC_013926.1; NC_002754.1; NC_013520.1;
NC_013520.1; NC_007181.1; NC_007426.1;
NC_003106.2; NC_020247.1; NC_020246.1;
NC_017276.1; NC_017275.1; NC_012632.1;
NC_012623.1; NC_012588.1; NC_011296.1;
NC_011296.1; NC_013769.1; NC_013769.1;
NC_012726.1; NC_021058.1; NC_012622.1;
NC_012589.1; NC_015151.1; NC_015954.1;
NC_004547.2; NC_000913.3; NC_010175.1;
NC_016070.1; NC_002950.2; NC_009380.1;
NC_009089.1; NC_009495.1; NC_022777.1;
NC_007796.1; NC_014106.1; NC_008054.1;
NC_002663.1; NC_013961.1; NC_011138.3;
NC_014228.1; NC_020990.1; NC_013892.1;
NC_015760.1; NC_009953.1; NC_009953.1;
NC_009439.1; NC_010572.1; NC_002971.3;
NC_021353.1; NC_014644.1; NC_010610.1;
NC_002935.2; NC_013722.1; NC_009464.1;
NC_007643.1; NC_002939.5; NC_010002.1;
NC_003198.1; NC_014318.1; NC_008526.1;
NC_011832.1; NC_008701.1; NC_007955.1;
NC_007796.1; NC_014147.1; NC_009053.1;
NC_014500.1; NC_016901.1; NC_015676.1;
NC_020418.1; NW_003613864.1; NC_000011.10;
NW_006800487.1; NC_006603.3; NW_003614246.1; NC_012602.1;
NC_013790.1; NC_009515.1; NC_015574.1;
NC_007871.1; NC_006478.3; NC_004347.2;
NC_009006.2; NT_078266.2; NC_003454.1;
NW_006212882.1; NC_011913.1; NC_009917.1;
NW_007675828.1; NW_007370782.1; NW_007248774.1;
NC_023642.1; NW_006775074.1;
NW_006730123.1; NW_006718075.1;
NW_006711808.1; NW_006400147.1;
NW_006408681.1; NW_006384369.1; NW_005882764.1;
NW_006200097.1; NC_022285.1;
NW_004209914.1; NC_018435.1; NC_018732.2;
NC_018165.1; NC_027879.1; NC_019830.1;
NC_013906.1; NC_009150.2; NC_000964.3;
NC_002696.2; NC_017034.1; NC_007581.1;
NC_018001.1; NC_017954.1; NC_014961.1;
NC_014961.1; NC_011766.1; NC_009776.1;
NC_007681.1; NC_007681.1; NC_005877.1;
NC_005877.1; NC_005877.1; NC_002689.2;
NC_000918.1; NC_000918.1; NC_021592.1;
NC_015931.1; NC_015931.1; NC_010482.1;
NC_009033.1; NC_000853.1; NC_015518.1;
NC_015416.1; NC_013849.1; NC_009440.1;
NC_009440.1; NC_009009.1; NC_007644.1;
NC_000917.1; NC_003106.2; NC_011916.1;
NC_007643.1; NC_006347.1; NC_004342.2;
NC_002945.3; NC_012589.1; NC_012623.1;
NC_011672.1; NC_016131.1; NW_004454187.1;
NC_019862.1; NC_010451.3; NC_015768.1;
NC_020173.2; NC_017972.1; NC_015320.1;
NC_011832.1; NC_010175.1; NC_010175.1;
NC_008599.1; NC_006461.1; NC_015637.1;
NC_009784.1; NC_016114.1; NC_001493.2;
NC_008508.1; NC_003197.1; NC_017844.1;
NW_006399893.1; NC_002695.1; NC_017634.1;
NC_003143.1; NC_017941.2; NC_004605.1;
NC_004605.1; NC_019411.1; NC_007164.1;
NC_002932.3; NC_005085.1; NC_027207.1;
NC_016452.1; NC_016112.1; NC_009784.1;
NC_007606.1; NC_002506.1; NC_018658.1;
NC_011751.1; NC_016497.1; NC_011678.1;
NC_006035.2; NC_026147.1; NC_013913.1;
NC_024132.1; NC_006461.1; NC_000913.3;
NC_014106.1; NC_008054.1; NC_013961.1;
NC_011138.3; NC_009778.1; NC_014228.1;
NC_020990.1; NC_013892.1; NC_015760.1;
NC_009953.1; NC_009953.1; NC_009439.1;
NC_010572.1; NC_014644.1; NC_010610.1;
NC_002935.2; NC_009464.1; NC_007643.1;
NC_002939.5; NC_003198.1; NC_014318.1;
NC_008526.1; NC_011832.1; NC_007955.1;
NC_007796.1; NC_015676.1; NC_015637.1;
NC_009784.1; NC_016114.1; NC_001493.2;
NC_008508.1; NC_003197.1; NC_017844.1;
NC_002695.1; NC_017941.2; NC_004605.1;
NC_007164.1; NC_002932.3; NC_016452.1;
NC_016112.1; NC_007606.1; NC_002506.1;
NC_018658.1; NC_011751.1; NC_026147.1;
NC_024132.1; NC_006461.1; NC_004547.2;
NC_002663.1; NC_002971.3; NC_013722.1;
NC_010002.1; NC_014147.1; NC_009053.1;
NC_014500.1; NC_016901.1; NC_020418.1;
NW_006399893.1; NC_017634.1; NC_003143.1;
NC_004605.1; NC_019411.1; NC_005085.1;
NC_027207.1; NC_009784.1; NC_016497.1;
NC_011678.1; NC_006035.2; NC_013913.1;
NC_000913.3; NC_002950.2; NC_019943.1;
NC_021355.1; NC_019942.1; NC_021058.1;
NC_017576.1; NC_009785.1; NC_019943.1;
NC_010175.1; NC_002754.1; NC_016070.1;
NC_003413.1; NC_003413.1; NC_021921.1;
NC_015516.1; NC_007681.1; NC_021169.1;
NC_020517.1; NC_018015.1; NC_017946.1;
NC_017946.1; NC_015865.1; NC_015847.1;
NC_015680.1; NC_015680.1; NC_015680.1;
NC_015474.1; NC_015435.1; NC_014106.1;
NC_013790.1; NC_012470.1; NC_010610.1;
NC_010610.1; NC_010482.1; NC_009515.1;
NC_009440.1; NC_008942.1; NC_008054.1;
NC_006624.1; NC_017459.1; NC_015518.1;
NC_014933.1; NC_013961.1; NC_013202.1;
NC_013158.1; NC_000917.1; NC_003901.1;
NC_003106.2; NC_009434.1; NC_005085.1;
NC_018224.1; NC_015943.1; NC_009380.1;
NC_006834.1; NC_003552.1; NC_020990.1;
NC_017276.1; NC_017275.1; NC_009615.1;
NC_012632.1; NC_012632.1; NC_012623.1;
NC_012588.1; NC_012588.1; NC_020800.1;
NC_019792.1; NC_015760.1; NC_015678.1;
NC_014222.1; NC_013887.1; NC_013887.1;
NC_013156.1; NC_013156.1; NC_009953.1;
NC_009634.1; NC_008618.1; NC_008599.1;
NC_007355.1; NC_002754.1; NC_002754.1;
NC_021082.1; NC_000868.1; NC_009089.1;
NC_009089.1; NC_020419.1; NC_021592.1;
NC_014644.1; NC_000916.1; NC_015474.1;
NC_014537.1; NC_012804.1; NC_016051.1;
NC_007644.1; NC_006624.1; NC_004557.1;
NC_004557.1; NC_017384.1; NC_016023.1;
NC_013722.1; NC_007643.1; NC_007643.1;
NC_016112.1; NC_016112.1; NC_014318.1;
NC_008526.1; NC_012726.1; NC_023044.1;
NC_022777.1; NC_014222.1; NC_011296.1;
NC_009953.1; NC_018001.1; NC_017954.1;
NC_022084.1; NC_018092.1; NC_018092.1;
NC_018015.1; NC_000961.1; NC_011567.1;
NC_000917.1; NC_015574.1; NC_014500.1;
NC_012622.1; NC_012589.1; NC_017274.1;
NC_015320.1; NC_014122.1; NC_021353.1;
NC_000916.1; NC_000961.1; NC_000961.1;
NC_015931.1; NC_013849.1; NC_011296.1;
NC_002754.1; NC_022084.1; NC_006590.3;
NW_003613770.1; NW_006770037.1;
NW_005882719.1; NW_006717982.1;
NW_003159449.1; NW_007676006.1;
NW_004454173.1; NC_009167.2; NC_021161.1;
NC_007131.6; NC_010443.4; NC_013929.1;
NC_016114.1; NC_020416.1; NC_014160.1;
NC_004342.2; NC_021366.1; NC_014430.1;
NW_007727498.1; NW_006777583.1; NW_006732012.1;
NW_006399890.1; NW_005882776.1;
NC_019874.1; NC_013918.1; NC_026607.2;
NC_022777.1; NC_019042.1; NC_011421.1;
NC_009784.1; NC_002695.1; NC_002737.2;
NC_021315.1; NC_007581.1; NC_022761.1;
NC_000918.1; NC_012883.1; NC_007297.1;
NC_004350.2; NC_003454.1; NC_000853.1;
NC_009464.1; NC_002932.3; NC_002932.3;
NC_016112.1; NC_006347.1; NC_004342.2;
NC_018658.1; NC_010397.1; NC_002678.2;
NC_011751.1; NC_011695.1; NC_025697.1;
NC_025205.1; NW_006239047.1;
NW_005372402.1; NC_020494.1; NW_003804572.1;
NC_016132.1; NC_027344.1; NC_025442.1;
NC_015320.1; NC_014923.1; NC_013046.1;
NC_011167.1; NC_007955.1; NC_002754.1;
NC_008818.1; NC_000918.1; NC_014222.1;
NC_011661.1; NC_003413.1; NC_010175.1;
NC_015474.1; NC_013202.1; NC_018224.1;
NC_003552.1; NC_009635.1; NC_007355.1;
NC_009089.1; NC_021592.1; NC_015847.1;
NC_009515.1; NC_018015.1; NC_006624.1;
NC_019943.1; NC_017459.1; NC_015943.1;
NC_015416.1; NC_022777.1; NC_022777.1;
NC_014222.1; NC_011296.1; NC_009634.1;
NC_022084.1; NC_018092.1; NC_000961.1;
NC_011567.1; NC_015574.1; NC_014122.1;
NC_021353.1; NC_015320.1; NC_014122.1;
NC_019471.1; NC_026149.1; NW_006711753.1;
NC_025697.1; NW_007540505.1; NW_006749413.1;
NC_008809.1; NT_033779.5;
NC_000961.1; NC_015562.1; NC_011645.1;
NC_017034.1; NC_019912.1; NC_011318.1;
NT_167064.1; NC_002939.5; NC_011904.1;
NC_008314.1; NC_024342.1; NW_007370677.1;
NW_006804181.1; NW_006711988.1;
NW_006399752.1; NW_006299715.1; NW_005785016.1;
NW_005805521.1; NC_018726.2; NC_018726.2;
NC_008801.1; NC_013081.1; NC_006840.2;
NC_023007.1; NC_014649.1; NC_007681.1;
NC_007681.1; NC_007681.1; NC_000918.1;
NC_021592.1; NC_013790.1; NC_003454.1;
NC_000853.1; NC_004557.1; NC_007644.1;
NW_007544598.1; NC_027207.1; NC_003901.1;
NC_009908.2; NC_016506.1; NC_006041.1;
NC_006029.1; NC_025997.1; NW_007733735.1;
NW_006799945.1; NW_006749276.1;
NC_016088.1; NW_005871062.1; NW_005369870.1;
NW_005372669.1; NW_004204575.1;
NC_021355.1; NC_014222.1; NC_009135.1;
NC_000868.1; NC_003413.1; NC_017946.1;
NC_015680.1; NC_015474.1; NC_012804.1;
NC_006624.1; NC_016070.1; NC_013887.1;
NC_013156.1; NC_022084.1; NC_018092.1;
NC_018001.1; NC_000961.1; NC_013849.1;
NC_016091.1; NC_013929.1; NC_018734.2;
NC_018609.1; NC_003413.1; NC_014961.1;
NC_018092.1; NC_021313.1; NC_013488.1;
NC_023186.1; NC_022777.1; NC_021921.1;
NC_013158.1; NC_019792.1; NC_004557.1;
NC_019943.1; NC_015516.1; NC_012470.1;
NC_016023.1; NC_009434.1; NC_005085.1;
NC_006834.1; NC_015678.1; NC_020419.1;
NC_020517.1; NC_007644.1; NC_017384.1;
NC_014933.1; NC_013722.1; NC_007643.1;
NC_016112.1; NC_009615.1; NC_002950.2;
NC_021082.1; NC_014500.1; NC_008804.1;
NC_007873.1; NC_004342.2; NC_018015.1;
NC_016901.1; NC_007796.1; NC_008195.1;
NC_013158.1; NC_014371.1; NC_019042.1;
NC_011044.1; NC_002946.2; NC_002946.2;
NC_002737.2; NC_018859.1; NC_026932.1;
NC_010610.1; NC_008942.1; NC_007297.1;
NC_004350.2; NC_004350.2; NC_002932.3;
NC_016112.1; NC_015574.1; NC_004342.2;
NC_014942.1; NC_026148.1; NC_025703.1;
NW_007680270.1; NC_020493.1;
NC_023554.1; NC_022327.1; NC_022331.1;
NC_022328.1; NC_016522.1; NC_002663.1;
NC_018609.1; NC_006448.1; NC_017576.1;
NC_015964.1; NC_002163.1; NC_002967.9;
NC_009785.1; NC_004829.2; NC_017576.1;
NC_002935.2; NC_013198.1; NC_020419.1;
NC_009613.3; NC_022246.1; NC_007643.1;
NC_018081.1; NC_007117.6; NC_023622.1;
NC_007297.1; NC_024462.1; NC_003076.8;
NC_019042.1; NC_002737.2; NC_004350.2;
NC_004116.1; NC_017946.1; NC_000961.1;
NT_167064.1; NC_002967.9; NC_012963.1;
NC_023658.1; NW_006725355.1; NW_006400033.1;
NW_006385373.1; NW_005815240.1;
NW_004197941.1; NC_009094.1; NC_019516.1;
NC_008599.1; NC_004740.1; NC_007929.1;
NC_006570.2; NC_014429.1; NC_026477.1;
NC_016462.1; NC_025688.1; NC_025693.1;
NC_024800.1; NW_006399825.1;
NC_019403.1; NC_016524.1; NC_007709.1;
NC_000007.14; NC_000962.3; NC_021058.1;
NC_021058.1; NC_002754.1; NC_017946.1;
NC_015680.1; NC_011529.1; NC_017276.1;
NC_012622.1; NC_012622.1; NC_000961.1;
NC_013520.1; NC_006448.1; NC_002950.2;
NC_017276.1; NC_017275.1; NC_012632.1;
NC_012588.1; NC_015636.1; NC_013887.1;
NC_013769.1; NC_013156.1; NC_009635.1;
NC_015680.1; NC_010482.1; NC_009698.1;
NC_012726.1; NC_015562.1; NC_011296.1;
NC_011296.1; NC_009634.1; NC_012589.1;
NC_012589.1; NC_013407.1; NC_013926.1;
NC_012622.1; NC_013156.1; NC_011296.1;
NC_013790.1; NC_009033.1; NC_016070.1;
NC_013849.1; NC_019977.1; NC_013407.1;
NC_015869.2; NC_011661.1; NC_000853.1;
NC_000917.1; NC_002945.3; NC_002754.1;
NC_007114.6; NC_019473.1; NC_023623.1;
NW_007577872.1; NC_013900.1;
NW_003155240.1; NC_000005.10; NC_002689.2;
NC_009009.1; NC_014222.1; NC_003413.1;
NC_014374.1; NC_011766.1; NC_000918.1;
NC_022093.1; NC_018092.1;
NC_000853.1; NC_017527.1; NC_015151.1;
NC_009495.1; NC_015562.1; NC_014222.1;
NC_015460.1; NC_015460.1;
NC_015460.1; NC_009440.1; NC_007643.1;
NC_003552.1; NW_006263392.1; AC_000177.1;
NC_004316.3; NC_016411.2; NC_012225.1;
NC_005788.4; NC_014323.1; NC_008818.1;
NC_017960.1; NW_006436284.1;
NW_006211808.1; NC_013743.1; NC_009380.1;
NW_005393620.1; NC_011089.1;
NW_007727769.1; NW_007673341.1; NC_024350.1;
NW_006921745.1; NW_006533007.1;
NW_006532497.1; NW_006712293.1; NW_006501110.1;
NW_006400054.1; NW_006408600.1;
NW_005882775.1; NW_005785850.1; NW_005812630.1;
NC_022312.1; NW_004454213.1; NC_018441.1;
NC_018723.2; NC_016133.1; NC_010446.4;
NC_009164.2; NC_003047.1; NC_007088.5;
NC_009776.1; NC_015931.1; NC_000916.1;
NC_015518.1; NC_007643.1; NC_003901.1;
NC_016491.1; NC_026938.1; NW_007379404.1;
NW_007377898.1; NC_008809.1; NC_016103.1;
NC_016099.1; NW_005372401.1;
NW_003140313.1; NW_003383754.1;
NC_008599.1; NC_016522.1; NC_014122.1;
NC_011167.1; NC_010175.1; NC_008698.1;
NC_007355.1; NC_005877.1; NC_003106.2;
NC_012623.1; NC_012589.1; NC_023069.1;
NC_008701.1; NC_018015.1; NC_007181.1;
NC_020247.1; NW_004218575.1; NC_022973.1;
NC_022984.1; NC_017459.1; NC_007426.1;
NC_002663.1; NC_016023.1; NW_006433950.1;
NW_006383296.1; NC_015636.1;
NC_015562.1; NC_015562.1; NC_015954.1;
NC_011832.1; NC_007796.1; NC_004547.2;
NC_002663.1; NC_010002.1; NC_015460.1;
NC_013722.1; NC_014147.1; NC_009053.1;
NC_016901.1; NC_014500.1; NC_020418.1;
NW_006890166.1; NC_012019.3; NC_017634.1;
NC_003143.1; NC_002971.3; NC_016477.1;
NC_025693.1; NC_004547.2; NC_002663.1;
NC_005085.1; NC_010002.1; NC_015460.1;
NC_020418.1; NC_013722.1; NC_014147.1;
NC_009053.1; NC_016901.1; NC_014500.1;
NC_000919.1; NC_009784.1; NW_006804308.1;
NC_017634.1; NC_003143.1; NC_004605.1;
NC_007284.2; NC_019472.1; NC_004547.2;
NC_009784.1; NC_020418.1; NC_002663.1;
NC_015460.1; NC_005085.1; NC_010002.1;
NC_002971.3; NC_013722.1; NC_014147.1;
NC_009053.1; NC_016901.1; NC_014500.1;
NC_017634.1; NC_003143.1; NC_004605.1;
NC_016457.1; NW_007546277.1; NC_014644.1;
NC_002939.5; NC_000913.3; NC_011138.3;
NC_014228.1; NC_013892.1; NC_009953.1;
NC_003198.1; NC_014106.1; NC_007643.1;
NC_007643.1; NC_016112.1; NC_014318.1;
NC_011832.1; NC_009953.1; NC_009439.1;
NC_007955.1; NC_014500.1; NC_015676.1;
NC_014106.1; NC_010610.1; NC_009440.1;
NC_008054.1; NC_007164.1; NC_015518.1;
NC_017276.1; NC_017275.1; NC_012632.1;
NC_012623.1; NC_012588.1; NC_015760.1;
NC_013769.1; NC_009953.1; NC_002754.1;
NC_010572.1; NC_012622.1; NC_012589.1;
NC_017274.1; NC_008054.1; NC_020990.1;
NC_015760.1; NC_009778.1; NC_008526.1;
NC_008481.2; AC_000180.1; NW_005842445.1;
NC_026935.1; NW_005784896.1;
NW_007359860.1; NC_013961.1; NC_010610.1;
NC_009440.1; NC_007164.1; NC_008508.1;
NC_010296.1; NC_003197.1; NW_008237220.1;
NW_005882971.1; NW_005853887.1;
NW_005394383.1; NC_018890.1; NC_018390.1;
NC_013051.1; NC_002695.1; NC_002677.1;
NC_009464.1; NC_002932.3; NC_018658.1;
NC_011751.1; NC_026504.1; NC_013486.1;
NW_005372181.1; NC_007796.1; NC_014644.1;
NC_002939.5; NC_000913.3; NC_011138.3

Meganucleases are by definition sequence-specific endonucleases with large (>14 bp) cleavage sites that can deliver DNA double-strand breaks at specific loci in living cells (Thierry and Dujon, Nucleic Acids Res., 1992, 20, 5625-5631). Meganucleases have been used to stimulate homologous recombination in the vicinity of their target sequences in cultured cells and plants (Rouet et al., Mol. Cell. Biol., 1994, 14, 8096-106; Choulika et al., Mol. Cell. Biol., 1995, 15, 1968-73; Donoho et al., Mol. Cell. Biol, 1998, 18, 4070-8; Elliott et al., Mol. Cell. Biol., 1998, 18, 93-101; Sargent et al., Mol. Cell. Biol., 1997, 17, 267-77; Puchta et al., Proc. Natl. Acad. Sci. USA, 1996, 93, 5055-60; Chiurazzi et al., Plant Cell, 1996, 8, 2057-2066), making meganuclease-induced recombination an efficient and robust method for genome engineering.

In nature, meganucleases are essentially represented by homing endonucleases (HEs), a family of endonucleases encoded by mobile genetic elements, whose function is to initiate DNA double-strand break-induced recombination events in a process referred to as homing (Chevalier and Stoddard, Nucleic Acids Res., 2001, 29, 3757-74; Kostriken et al., Cell; 1983, 35, 167-74; Jacquier and Dujon, Cell, 1985, 41, 383-94). Several hundreds of HEs have been identified in bacteria, eukaryotes, and archea (Chevalier and Stoddard, Nucleic Acids Res., 2001, 29, 3757-74). In addition to those found in nature, meganucleases suitable for practicing the methods of the present disclosure may also be functional variants or functional fragments of wild-type meganucleases.

Transcription activator-like effector nucleases, or “TALEN,” are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence, so when combined with a nuclease, DNA can be cut at specific locations (Boch, J., Nature Biotechnology, 29 (2):135-6).

A transposon, or a transposable element (TE) or jumping gene, is a DNA sequence that can change its position within a genome, sometimes creating or reversing mutations and altering the cell's genetic identity and genome size (Bourque et al. (2018) Genome Biology, 19 (1): 199). There are at least two classes of transposons, Class I transposons or retrotransposons generally function via reverse transcription and Class II transposons or DNA transposons encode the protein transposase, which they require for insertion and excision, and some of these transposons also encode other proteins (Pray, L. A. (2008) Nature Education, 1(1): 204). Transposons suitable for practicing the methods of the present disclosure may comprise not only wild-type transposons, but also functional fragments thereof.

Zinc-finger nucleases, or ZFNs, are artificial restriction enzymes generated by fusing a zinc finger DNA-binding domain to a DNA-cleavage domain. The modular DNA recognition preferences of zinc-fingers protein allows for the rational design of site-specific multi-finger DNA binding proteins. Fusion of the nuclease domain from the Type II restriction enzyme Fok I to site-specific zinc-finger proteins allows for the creation of site-specific nucleases. See, e.g., US 2003/0232410; US 2005/0208489; US 2005/0026157; US 2005/0064474; US 2006/0188987; US 2006/0063231; US 2007/0134796; US 2008/015164; US 2008/0131962; US 2008/015996; WO 07/014275; and WO 2008/133938, which all describe use of zinc-finger nucleases.

Recombinases are genetic recombination enzymes. Exemplary recombinases include, but not limited to, site-specific recombinases which are derived from bacteria and fungi and catalyze directionally sensitive DNA exchange reactions between short (30-40 nucleotides) target site sequences that are specific to each recombinase. These reactions enable excision/insertion, inversion, translocation, and cassette exchange (Nern et al. (2011) PNAS, 108(34):14198-203; Garcia-Otin et al. (2006) Frontiers in Bioscience, 11:1108-36; Dymecki et al. (2007) Neuron, 54(1):17-34; Luan et al. (2007) Current Opinion in Neurobiology, 17(5):572-80; Fenno et al. (2014) Nature Methods, 11(7):763-72). Recombinases suitable for practicing the methods of the present disclosure may comprise not only wild-type recombinases, but also functional fragments thereof.

A “functional fragment” of a protein, polypeptide or nucleic acid is a protein, polypeptide or nucleic acid whose sequence is not identical to the full-length protein, polypeptide or nucleic acid, yet retains some or all of the function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined by, for instance, filter-binding, electrophoretic mobility-shift, or immunoprecipitation assays. DNA cleavage can be assayed by gel electrophoresis. See e.g., Ausubel et al., Current Protocols in Molecular Biology, John Wiley & Sons, New York, 1987 and periodic updates. The ability of a protein to interact with another protein can be determined by, for example, co-immunoprecipitation, two-hybrid assays or complementation, both genetic and biochemical. See e.g., Fields et al. (1989) Nature 340:245-246; U.S. Pat. No. 5,585,245 and WO 98/44350.

The term “plasmid” refers to a DNA molecule within a cell that is physically separated from chromosomal DNA and can replicate independently. Thus, a plasmid is considered a replicon or a unit of DNA capable of replicating autonomously within a suitable host.

The term “hydrogel” refers to a network of polymer chains that are hydrophilic, sometimes found as a colloidal gel in which water is the dispersion medium. In some embodiments, the hydrogel suitable for practicing the present disclosure is Matrigel, which is a trade name for a gelatinous protein mixture secreted by Engelbreth-Holm-Swarm (EHS) mouse sarcoma cells produced and marketed by Corning Life Sciences and BD Biosciences. The main components of Matrigel® are structural proteins such as laminin, nidogen, collagen and heparan sulfate proteoglycans which present cultured cells with the adhesive peptide sequences that they would encounter in their natural environment (Hughes et al. (2010) Proteomics, 10(9):1886-90). Also present are growth factors like TGF-β and EGF that prevent differentiation and promote proliferation of many cell types.

As used herein, the terms “squamous cell carcinoma” or “SCC” means a cancer of a kind of epithelial cell, the squamous cell that make up the main component or part of the epidermis of the skin.

SCC is one of the major forms of skin cancer. However, squamous cells also occur in the lining of the digestive tract, lungs, and other areas of the body, and SCC occurs as a form of cancer in diverse tissues, including the lips, mouth, esophagus, urinary bladder, prostate, lung, vagina, and cervix, among others. SCC is a histologically distinct form of cancer arising from the uncontrolled multiplication of cells of epithelium, or cells showing particular cytological or tissue architectural characteristics of squamous cell differentiation, such as the presence of keratin, tonofilament bundles, or desmosomes, structures involved in cell-to-cell adhesion. SCC is sometimes referred to as “epidermoid carcinoma” and “squamous cell epithelioma.”

Genome-Edited Keratinocytes

The disclosure relates to a composition comprising one or a plurality of genome-edited keratinocytes. In some embodiment, the keratinocyte is a primary cell from a human or mammal. Genome-edited, or genetically altered, keratinocytes can be prepared by exposing primary keratinocytes to one or a plurality of mutagens for a time period sufficient to mutate one or a plurality of wild-type genes such that genome-edited primary keratinocytes deficient in expression of the one or plurality of wild-type genes are produced. Mutagens suitable for causing genetic mutation(s) can be any type of physical or chemical mutagens known in the art, such as radioactive substances, radiation, and chemical substances. Mutagens can also be molecules that frequently used in laboratories to introduce genetic mutation(s), such as vectors or gene-modifying enzymes. In some embodiments, the mutagen is a gene-modifying enzyme, such as a Cas protein, a meganuclease, a transcription activator-like effector nucleases (TALEN), a transposon, a zinc-finger nuclease (ZFN), or a recombinase, but not limited thereto. In some embodiments, the mutagen is a vector, such as a plasmid, but not limited thereto.

In some embodiments, the genome-edited keratinocytes according to the present disclosure are deficient in expression of one or a plurality of wild-type genes, particularity those provided in Table 1.

TABLE 1
List of target genes for genome editing.

| Gene | Genomic Sequence | Target Transcript |
|---|---|---|
| TP53 | NC_000017.11 | NM_000546.5 |
| NOTCH1 | NC_000009.12 | NM_017617.3 |
| CDKN2A | NC_000009.12 | NM_001195132.1 |
| TP63 | NC_000003.12 | NM_001114980.1 |
| FAT1 | NC_000004.12 | NM_005245.3 |
| CASP8 | NC_000002.12 | NM_001080125.1 |
| RB1 | NC_000013.11 | NM_000321.2 |
| PTEN | NC_000010.11 | NM_001304718.1 |
| NSD1 | NC_000005.10 | NM_022455.4 |
| KMT2D | NC_000012.12 | NM_003482.3 |
| AJUBA | NC_000014.9 | NM_032876.5 |
| EP300 | NC_000022.11 | NM_001429.3 |
| EPHA2 | NC_000001.11 | NM_004431.3 |
| FBXW7 | NC_000004.12 | NM_033632.3 |
| RASA1 | NC_000005.10 | NM_002890.2 |
| TGFBR2 | NC_000003.12 | NM_003242.5 |
| ZNF750 | NC_000017.11 | NM_024702.2 |
| KEAP1 | NC_000019.10 | NM_203500.1 |
| HRAS | NC_000011.10 | NM_001130442.1 |
| PIK3CA | NC_000003.12 | NM_006218.2 |
| RHOA | NC_000003.12 | NM_001664.2 |
| CCR5 | NC_000003.12 | NM_000579.3 |
| AAVS1 | NC_000019.10 | NM_017607.4 |

In some embodiments, the genome-edited keratinocytes according to the present disclosure are deficient in expression of a single wild-type gene. In some embodiments, the genome-edited keratinocytes according to the present disclosure are deficient in expression of a single wild-type gene chosen from Table 1. In some embodiments, the genome-edited keratinocytes according to the present disclosure are deficient in expression of wild type TP53 gene. In some embodiments, the genome-edited keratinocytes according to the present disclosure are deficient in expression of wild-type CDKN2A gene. In some embodiments, the genome-edited keratinocytes according to the present disclosure are deficient in expression of wild-type NOTCH1 gene.

In some embodiments, the genome-edited keratinocytes according to the present disclosure are deficient in expression of at least two wild-type genes. In some embodiments, the genome-edited keratinocytes according to the present disclosure are deficient in expression of at least one wild-type gene chosen from Table 1 and at least one further wild-type gene. In some embodiments, the genome-edited keratinocytes according to the present disclosure are deficient in expression of wild type TP53 gene and at least one further wild-type gene. In some embodiments, the genome-edited keratinocytes according to the present disclosure are deficient in expression of wild type CDKN2A gene and at least one further wild-type gene. In some embodiments, the genome-edited keratinocytes according to the present disclosure are deficient in expression of wild type NOTCH1 gene and at least one further wild-type gene. In some embodiments, the genome-edited keratinocytes according to the present disclosure are deficient in expression of at least two wild-type genes chosen from Table 1. In some embodiments, the genome-edited keratinocytes according to the present disclosure are deficient in expression of wild type TP53 and CDKN2A genes. In some embodiments, the genome-edited keratinocytes according to the present disclosure are deficient in expression of wild type TP53 and NOTCH1 genes. In some embodiments, the genome-edited keratinocytes according to the present disclosure are deficient in expression of wild type CDKN2A and NOTCH1 genes.

In some embodiments, the genome-edited keratinocytes according to the present disclosure are deficient in expression of at least three wild-type genes. In some embodiments, the genome-edited keratinocytes according to the present disclosure are deficient in expression of at least one wild-type gene chosen from Table 1 and at least two further wild-type gene. In some embodiments, the genome-edited keratinocytes according to the present disclosure are deficient in expression of wild type TP53 gene and two further wild-type genes. In some embodiments, the genome-edited keratinocytes according to the present disclosure are deficient in expression of wild type CDKN2A gene and two further wild-type genes. In some embodiments, the genome-edited keratinocytes according to the present disclosure are deficient in expression of wild type NOTCH1 gene and two further wild-type genes. In some embodiments, the genome-edited keratinocytes according to the present disclosure are deficient in expression of at least two wild-type genes chosen from Table 1 and at least one further wild-type gene. In some embodiments, the genome-edited keratinocytes according to the present disclosure are deficient in expression of wild type TP53 and CDKN2A genes and one further wild-type gene. In some embodiments, the genome-edited keratinocytes according to the present disclosure are deficient in expression of wild type TP53 and NOTCH1 genes and one further wild-type gene. In some embodiments, the genome-edited keratinocytes according to the present disclosure are deficient in expression of wild type CDKN2A and NOTCH1 genes and one further wild-type gene. In some embodiments, the genome-edited keratinocytes according to the present disclosure are deficient in expression of at least three wild-type genes chosen from Table 1. In some embodiments, the genome-edited keratinocytes according to the present disclosure are deficient in expression of wild type TP53, CDKN2A and NOTCH1 genes.

In some embodiments, the genome-edited keratinocytes according to the present disclosure are deficient in expression of four or more wild-type genes. In some embodiments, the genome-edited keratinocytes according to the present disclosure are deficient in expression of at least one wild-type gene chosen from Table 1 and at least three or more wild-type genes. In some embodiments, the genome-edited keratinocytes according to the present disclosure are deficient in expression of wild type TP53 gene and three or more wild type genes. In some embodiments, the genome-edited keratinocytes according to the present disclosure are deficient in expression of wild type CDKN2A gene and three or more wild type genes. In some embodiments, the genome-edited keratinocytes according to the present disclosure are deficient in expression of wild type NOTCH1 gene and three or more wild type genes. In some embodiments, the genome-edited keratinocytes according to the present disclosure are deficient in expression of at least two wild-type genes chosen from Table 1 and at least tow or more wild-type genes. In some embodiments, the genome-edited keratinocytes according to the present disclosure are deficient in expression of wild type TP53 and CDKN2A genes and two or more wild type genes. In some embodiments, the genome-edited keratinocytes according to the present disclosure are deficient in expression of wild type TP53 and NOTCH1 genes and two or more wild type genes. In some embodiments, the genome-edited keratinocytes according to the present disclosure are deficient in expression of wild type CDKN2A and NOTCH1 genes and two or more wild type genes. In some embodiments, the genome-edited keratinocytes according to the present disclosure are deficient in expression of at least three wild-type gene chosen from Table 1 and at least one or more wild-type genes. In some embodiments, the genome-edited keratinocytes according to the present disclosure are deficient in expression of wild type TP53, CDKN2A and NOTCH1 genes and one or more wild type genes. In some embodiments, the genome-edited keratinocytes according to the present disclosure are deficient in expression of four or more wild-type genes chosen from Table 1.

In some embodiments, the genome-edited keratinocytes according to the present disclosure comprises at least one mutated gene having at least about 70%, 75%, 80%, 85%, 90%, 95%, 96%, 97%, 98%, 99% or more sequence identity to SEQ ID NO: 311 or 312, provided that it does not comprise the nucleic acid sequence of SEQ ID NO: 307. In some embodiments, the genome-edited keratinocytes according to the present disclosure comprises at least one mutated gene comprising the nucleic acid sequence of SEQ ID NO: 311 or 312. In some embodiments, the genome-edited keratinocytes according to the present disclosure comprises at least one mutated gene having at least about 70%, 75%, 80%, 85%, 90%, 95%, 96%, 97%, 98%, 99% or more sequence identity to SEQ ID NO: 313 or 314, provided that it does not comprise the nucleic acid sequence of SEQ ID NO: 308. In some embodiments, the genome-edited keratinocytes according to the present disclosure comprises at least one mutated gene comprising the nucleic acid sequence of SEQ ID NO: 313 or 314. In some embodiments, the genome-edited keratinocytes according to the present disclosure comprises at least one mutated gene having at least about 70%, 75%, 80%, 85%, 90%, 95%, 96%, 97%, 98%, 99% or more sequence identity to SEQ ID NO: 315 or 316, provided that it is free of the nucleic acid sequence of SEQ ID NO: 309. In some embodiments, the genome-edited keratinocytes according to the present disclosure comprises at least one mutated gene comprising the nucleic acid sequence of SEQ ID NO: 315 or 316.

In some embodiments, the genome-edited keratinocytes according to the present disclosure can be prepared by transfecting primary keratinocytes with a plasmid, cosmid or nucleic acid sequence complexed with protein. Primary keratinocytes can be isolated from skin tissue or mucosa, such as oral mucosa, mucosa of the upper aerodigestive tract, esophageal mucosa, vaginal mucosa, or cervical mucosa, using any method known in the art or those disclosed herein. Transfection of primary keratinocytes can be performed using any method known in the art. In some embodiments, the transfection of primary keratinocytes are performed by seeding primary keratinocytes on tissue culture-treated polystyrene multi-well plates at a density of about 100,000, 110,000, 120,000, 130,000, 140,000, 150,000, 160,000, 170,000, 180,000, 190,000, 200,000 or more cells per well or per milliliter of volume, followed by incubation with plasmid for transfection for about 2, 4, 6, 8, 12, 24, 36, 48 or more hours.

In some embodiments, the primary keratinocytes are transfected with one or a plurality of plasmids or vectors. In some embodiments, the primary keratinocytes are transfected with one or a plurality of ribonucleoprotein (RNP) complexes. In some embodiments, the primary keratinocytes are transfected with one or a plurality of CRISPR/Cas9 ribonucleoprotein (RNP) complexes. In some embodiments, the transfection of primary keratinocytes are carried out by electroporation. This can be accomplished using electroporation devices that are configured to deliver to primary keratinocytes pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch.

The amount of nucleic acid (e.g., ssDNA, dsDNA, RNA) that may be introduced into the primary keratinocytes can be from about 0.5 nanograms to about 50 micrograms. However, this amount may be varied to optimize transfection efficiency and/or cell viability. In some embodiments, less than about 100 picograms of nucleic acid may be added to each cell sample (e.g., one or more cells being electroporated). In some embodiments, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid may be added to each cell sample (e.g., one or more cells being electroporated).

Electroporation using, for example, the Neon® Transfection System (ThermoFisher Scientific), the AMAXA® Nucleofector (AMAXA Biosystems), or the MaxCyte ATx instrument (MAXCYTE, Inc.) can also be used for delivery of nucleic acids into primary keratinocytes. Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some embodiments, the electroporation pulse voltage, the electroporation pulse width, number of pulses, cell density, and tip type may be adjusted to optimize transfection efficiency and/or cell viability.

In some embodiments, electroporation pulse voltage may be varied to optimize transfection efficiency and/or cell viability. In some embodiments, the electroporation voltage may be less than about 500 volts. In some embodiments, the electroporation voltage may be at least about 500 volts, at least about 600 volts, at least about 700 volts, at least about 800 volts, at least about 900 volts, at least about 1000 volts, at least about 1100 volts, at least about 1200 volts, at least about 1300 volts, at least about 1400 volts, at least about 1500 volts, at least about 1600 volts, at least about 1700 volts, at least about 1800 volts, at least about 1900 volts, at least about 2000 volts, at least about 2100 volts, at least about 2200 volts, at least about 2300 volts, at least about 2400 volts, at least about 2500 volts, at least about 2600 volts, at least about 2700 volts, at least about 2800 volts, at least about 2900 volts, or at least about 3000 volts.

In some embodiments, electroporation pulse width may be varied to optimize transfection efficiency and/or cell viability. In some embodiments, any of the methods disclosed herein comprise the electroporation pulse width may be less than about 5 milliseconds. In some embodiments, the electroporation width may be at least about 5 milliseconds, at least about 6 milliseconds, at least about 7 milliseconds, at least about 8 milliseconds, at least about 9 milliseconds, at least about 10 milliseconds, at least about 11 milliseconds, at least about 12 milliseconds, at least about 13 milliseconds, at least about 14 milliseconds, at least about 15 milliseconds, at least about 16 milliseconds, at least about 17 milliseconds, at least about 18 milliseconds, at least about 19 milliseconds, at least about 20 milliseconds, at least about 21 milliseconds, at least about 22 milliseconds, at least about 23 milliseconds, at least about 24 milliseconds, at least about 25 milliseconds, at least about 26 milliseconds, at least about 27 milliseconds, at least about 28 milliseconds, at least about 29 milliseconds, at least about 30 milliseconds, at least about 31 milliseconds, at least about 32 milliseconds, at least about 33 milliseconds, at least about 34 milliseconds, at least about 35 milliseconds, at least about 36 milliseconds, at least about 37 milliseconds, at least about 38 milliseconds, at least about 39 milliseconds, at least about 40 milliseconds, at least about 41 milliseconds, at least about 42 milliseconds, at least about 43 milliseconds, at least about 44 milliseconds, at least about 45 milliseconds, at least about 46 milliseconds, at least about 47 milliseconds, at least about 48 milliseconds, at least about 49 milliseconds, or at least about 50 milliseconds.

In some embodiments, the number of electroporation pulses may be varied to optimize transfection efficiency and/or cell viability. In some embodiments, electroporation may comprise a single pulse. In some embodiments, electroporation may comprise more than one pulse. In some embodiments, electroporation may comprise 2 pulses, 3 pulses, 4 pulses, 5 pulses 6 pulses, 7 pulses, 8 pulses, 9 pulses, or 10 or more pulses.

In some embodiments, the starting cell density for electroporation may be varied to optimize transfection efficiency and/or cell viability. In some embodiments, the starting cell density for electroporation may be less than about 1×10⁵ cells. In some embodiments, the starting cell density for electroporation may be at least about 1×10⁵ cells, at least about 2×10⁵ cells, at least about 3×10⁵ cells, at least about 4×10⁵ cells, at least about 5×10⁵ cells, at least about 6×10⁵ cells, at least about 7×10⁵ cells, at least about 8×10⁵ cells, at least about 9×10⁵ cells, at least about 1×10⁶ cells, at least about 1.5×10⁶ cells, at least about 2×10⁶ cells, at least about 2.5×10⁶ cells, at least about 3×10⁶ cells, at least about 3.5×10⁶ cells, at least about 4×10⁶ cells, at least about 4.5×10⁶ cells, at least about 5×10⁶ cells, at least about 5.5×10⁶ cells, at least about 6×10⁶ cells, at least about 6.5×10⁶ cells, at least about 7×10⁶ cells, at least about 7.5×10⁶ cells, at least about 8×10⁶ cells, at least about 8.5×10⁶ cells, at least about 9×10⁶ cells, at least about 9.5×10⁶ cells, at least about 1×10⁷ cells, at least about 1.2×10⁷ cells, at least about 1.4×10⁷ cells, at least about 1.6×10⁷ cells, at least about 1.8×10⁷ cells, at least about 2×10⁷ cells, at least about 2.2×10⁷ cells, at least about 2.4×10⁷ cells, at least about 2.6×10⁷ cells, at least about 2.8×10⁷ cells, at least about 3×10⁷ cells, at least about 3.2×10⁷ cells, at least about 3.4×10⁷ cells, at least about 3.6×10⁷ cells, at least about 3.8×10⁷ cells, at least about 4×10⁷ cells, at least about 4.2×10⁷ cells, at least about 4.4×10⁷ cells, at least about 4.6×10⁷ cells, at least about 4.8×10⁷ cells, or at least about 5×10⁷ cells.

Additional exemplary nucleic acid delivery systems include those provided by AMAXA® Biosystems (Cologne, Germany), Life Technologies (Frederick, Md.), MAXCYTE, Inc. (Rockville, Md.), BTX Molecular Delivery Systems (Holliston, Mass.) and Copernicus Therapeutics Inc. (see for example U.S. Pat. No. 6,008,336).

The transfection efficiency of primary keratinocytes with any of the nucleic acid delivery platforms described herein, for example, electroporation or lipofection, can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%. The cell viability and/or transfection efficiency may be measured using any method known in the art, including but not limited to, trypan blue exclusion, terminal deoxynucleotidyl transferase dUTP nick end labeling (TUNEL), the presence or absence of given cell-surface markers (e.g., CD4 or CD8), telomere length, fluorescence-activated cell sorting (FACS), real-time PCR, or droplet digital PCR. In some embodiments, FACS may be used to detect the cell viability and/or transfection efficiency. In other embodiments, apoptosis of cells may be measured using a TUNEL assay.

In some embodiments, transfected primary keratinocytes can be selected and separated from non-transfected primary keratinocytes. However, selection and separation of transfected primary keratinocytes is not an essential step of the methods disclosed herein. In some embodiments therefore, the methods of the disclosure do not include a step of selecting and/or sorting transfected primary keratinocytes. When this selection step is used, transfected keratinocytes can be selected by FACS in some embodiments. In some embodiments, transfected keratinocytes can be sorted onto tissue culture-treated polystyrene multi-well plates.

Depending on the transfection efficiency and whether transfected primary keratinocytes are selected and sorted, a population of genome-edited primary keratinocytes according to the disclosure may comprise non-transfected primary keratinocytes. In some embodiments, a population of genome-edited primary keratinocytes according to the disclosure comprises from about 50% to about 100% of transfected primary keratinocytes. In some embodiments, a population of genome-edited primary keratinocytes according to the disclosure comprises from about 45% to about 95% of transfected primary keratinocytes. In some embodiments, a population of genome-edited primary keratinocytes according to the disclosure comprises from about 40% to about 90% of transfected primary keratinocytes. In some embodiments, a population of genome-edited primary keratinocytes according to the disclosure comprises from about 35% to about 85% of transfected primary keratinocytes. In some embodiments, a population of genome-edited primary keratinocytes according to the disclosure comprises from about 30% to about 80% of transfected primary keratinocytes. In some embodiments, a population of genome-edited primary keratinocytes according to the disclosure comprises from about 25% to about 75% of transfected primary keratinocytes. In some embodiments, a population of genome-edited primary keratinocytes according to the disclosure comprises from about 20% to about 70% of transfected primary keratinocytes. In some embodiments, a population of genome-edited primary keratinocytes according to the disclosure comprises from about 15% to about 65% of transfected primary keratinocytes. In some embodiments, a population of genome-edited primary keratinocytes according to the disclosure comprises from about 10% to about 60% of transfected primary keratinocytes. In some embodiments, a population of genome-edited primary keratinocytes according to the disclosure comprises about 100% of transfected primary keratinocytes. In some embodiments, a population of genome-edited primary keratinocytes according to the disclosure comprises about 95% of transfected primary keratinocytes. In some embodiments, a population of genome-edited primary keratinocytes according to the disclosure comprises about 90% of transfected primary keratinocytes. In some embodiments, a population of genome-edited primary keratinocytes according to the disclosure comprises about 85% of transfected primary keratinocytes. In some embodiments, a population of genome-edited primary keratinocytes according to the disclosure comprises about 80% of transfected primary keratinocytes. In some embodiments, a population of genome-edited primary keratinocytes according to the disclosure comprises about 75% of transfected primary keratinocytes. In some embodiments, a population of genome-edited primary keratinocytes according to the disclosure comprises about 70% of transfected primary keratinocytes. In some embodiments, a population of genome-edited primary keratinocytes according to the disclosure comprises about 65% of transfected primary keratinocytes. In some embodiments, a population of genome-edited primary keratinocytes according to the disclosure comprises about 60% of transfected primary keratinocytes. In some embodiments, a population of genome-edited primary keratinocytes according to the disclosure comprises about 55% of transfected primary keratinocytes. In some embodiments, a population of genome-edited primary keratinocytes according to the disclosure comprises about 50% of transfected primary keratinocytes. In some embodiments, a population of genome-edited primary keratinocytes according to the disclosure comprises about 45%, 40%, 35%, 30%, 25%, 20% 15%, 10%, 5% or less than about 5% of transfected primary keratinocytes.

In some embodiments, the plasmid for transfection comprises a nucleic acid sequence encoding a gene-modifying enzyme. Any gene-modifying enzyme known in the art can be used. Examples of gene-modifying enzymes include, but not limited to Cas proteins, meganucleases, transcription activator-like effector nucleases (TALENs), transposons, zinc-finger nucleases (ZFNs), or recombinases, or functional fragments thereof. A functional fragment of such a gene-modifying enzyme may comprise an amino acid sequence having at least about 75%, 80%, 85%, 90%, 91%, 92%, 93%, 94%, 95%, 96%, 97%, 98%, or more sequence identity to the parent gene-modifying enzyme. Representatives of such gene-modifying enzymes are provided in Table 2. Functional fragments of these representative gene-modifying enzymes therefore may comprise an amino acid sequence having at least about 75%, 80%, 85%, 90%, 91%, 92%, 93%, 94%, 95%, 96%, 97%, 98%, 99% or more sequence identity to any of SEQ ID NO: 16, 17, 18, 19, 20, 21, 22, 23, 24, 25, 26, 27, 28, 29 or 30.

TABLE 2
Representatives of gene-modifying enzymes.

| | | SEQ |
|---|---|---|
| | | ID |
| Name | Sequence | NO |
| Cas9 | MDKKYSIGLDIGTNSVGWAVITDEY | 16 |
| | KVPSKKFKVLGNTDRHSIKKNLIGA | |
| | LLFDSGETAEATRLKRTARRRYTRR | |
| | KNRICYLQEIFSNEMAKVDDSFFHR | |
| | LEESFLVEEDKKHERHPIFGNIVDE | |
| | VAYHEKYPTIYHLRKKLVDSTDKAD | |
| | LRLIYLALAHMIKFRGHFLIEGDLN | |
| | PDNSDVDKLFIQLVQTYNQLFEENP | |
| | INASGVDAKAILSARLSKSRRLENL | |
| | IAQLPGEKKNGLFGNLIALSLGLTP | |
| | NFKSNFDLAEDAKLQLSKDTYDDDL | |
| | DNLLAQIGDQYADLFLAAKNLSDAI | |
| | LLSDILRVNTEITKAPLSASMIKRY | |
| | DEHHQDLTLLKALVRQQLPEKYKEI | |
| | FFDQSKNGYAGYIDGGASQEEFYKF | |
| | IKPILEKMDGTEELLVKLNREDLLR | |
| | KQRTFDNGSIPHQIHLGELHAILRR | |
| | QEDFYPFLKDNREKIEKILTFRIPY | |
| | YVGPLARGNSRFAWMTRKSEETITP | |
| | WNFEEVVDKGASAQSFIERMTNFDK | |
| | NLPNEKVLPKHSLLYEYFTVYNELT | |
| | KVKYVTEGMRKPAFLSGEQKKAIVD | |
| | LLFKTNRKVTVKQLKEDYFKKIECF | |
| | DSVEISGVEDRFNASLGTYHDLLKI | |
| | IKDKDFLDNEENEDILEDIVLTLTL | |
| | FEDREMIEERLKTYAHLFDDKVMKQ | |
| | LKRRRYTGWGRLSRKLINGIRDKQS | |
| | GKTILDFLKSDGFANRNFMQLIHDD | |
| | SLTFKEDIQKAQVSGQGDSLHEHIA | |
| | NLAGSPAIKKGILQTVKVVDELVKV | |
| | MGRHKPENIVIEMARENQTTQKGQK | |
| | NSRERMKRIEEGIKELGSQILKEHP | |
| | VENTQLQNEKLYLYYLQNGRDMYVD | |
| | QELDINRLSDYDVDHIVPQSFLKDD | |
| | SIDNKVLTRSDKNRGKSDNVPSEEV | |
| | VKKMKNYWRQLLNAKLITQRKFDNL | |
| | TKAERGGLSELDKAGFIKRQLVETR | |
| | QITKHVAQILDSRMNTKYDENDKLI | |
| | REVKVITLKSKLVSDFRKDFQFYKV | |
| | REINNYHHAHDAYLNAVVGTALIKK | |
| | YPKLESEFVYGDYKVYDVRKMIAKS | |
| | EQEIGKATAKYFFYSNIMNFFKTEI | |
| | TLANGEIRKRPLIETNGETGEIVWD | |
| | KGRDFATVRKVLSMPQVNIVKKTEV | |
| | QTGGFSKESILPKRNSDKLIARKKD | |
| | WDPKKYGGFDSPTVAYSVLVVAKVE | |
| | KGKSKKLKSVKELLGITIMERSSFE | |
| | KNPIDFLEAKGYKEVKKDLIIKLPK | |
| | YSLFELENGRKRMLASAGELQKGNE | |
| | LALPSKYVNFLYLASHYEKLKGSPE | |
| | DNEQKQLFVEQHKHYLDEIIEQISE | |
| | FSKRVILADANLDKVLSAYNKHRDK | |
| | PIREQAENIIHLFTLTNLGAPAAFK | |
| | YFDTTIDRKRYTSTKEVLDATLIHQ | |
| | SITGLYETRIDLSQLGGDSRADPKK | |
| | KRKV | |
| Cas9_ST | MDKKYSIGLDIGTNSVGWAVITDEY | 17 |
| RP1 | KVPSKKFKVLGNTDRHSIKKNLIGA | |
| | LLFDSGETAEATRLKRTARRRYTRR | |
| | KNRICYLQEIFSNEMAKVDDSFFHR | |
| | LEESFLVEEDKKHERHPIFGNIVDE | |
| | VAYHEKYPTIYHLRKKLVDSTDKAD | |
| | LRLIYLALAHMIKFRGHFLIEGDLN | |
| | PDNSDVDKLFIQLVQTYNQLFEENP | |
| | INASGVDAKAILSARLSKSRRLENL | |
| | IAQLPGEKKNGLFGNLIALSLGLTP | |
| | NFKSNFDLAEDAKLQLSKDTYDDDL | |
| | DNLLAQIGDQYADLFLAAKNLSDAI | |
| | LLSDILRVNTEITKAPLSASMIKRY | |
| | DEHHQDLTLLKALVRQQLPEKYKEI | |
| | FFDQSKNGYAGYIDGGASQEEFYKF | |
| | IKPILEKMDGTEELLVKLNREDLLR | |
| | KQRTFDNGSIPHQIHLGELHAILRR | |
| | QEDFYPFLKDNREKIEKILTFRIPY | |
| | YVGPLARGNSRFAWMTRKSEETITP | |
| | WNFEEVVDKGASAQSFIERMTNFDK | |
| | NLPNEKVLPKHSLLYEYFTVYNELT | |
| | KVKYVTEGMRKPAFLSGEQKKAIVD | |
| | LLFKTNRKVTVKQLKEDYFKKIECF | |
| | DSVEISGVEDRFNASLGTYHDLLKI | |
| | IKDKDFLDNEENEDILEDIVLTLTL | |
| | FEDREMIEERLKTYAHLFDDKVMKQ | |
| | LKRRRYTGWGRLSRKLINGIRDKQS | |
| | GKTILDFLKSDGFANRNFMQLIHDD | |
| | SLTFKEDIQKAQVSGQGDSLHEHIA | |
| | NLAGSPAIKKGILQTVKVVDELVKV | |
| | MGRHKPENIVIEMARENQTTQKGQK | |
| | NSRERMKRIEEGIKELGSQILKEHP | |
| | VENTQLQNEKLYLYYLQNGRDMYVD | |
| | QELDINRLSDYDVDHIVPQSFLKDD | |
| | SIDNKVLTRSDKNRGKSDNVPSEEV | |
| | VKKMKNYWRQLLNAKLITQRKFDNL | |
| | TKAERGGLSELDKAGFIKRQLVETR | |
| | QITKHVAQILDSRMNTKYDENDKLI | |
| | REVKVITLKSKLVSDFRKDFQFYKV | |
| | REINNYHHAHDAYLNAVVGTALIKK | |
| | YPKLESEFVYGDYKVYDVRKMIAKS | |
| | EQEIGKATAKYFFYSNIMNFFKTEI | |
| | TLANGEIRKRPLIETNGETGEIVWD | |
| | KGRDFATVRKVLSMPQVNIVKKTEV | |
| | QTGGFSKESILPKRNSDKLIARKKD | |
| | WDPKKYGGFDSPTVAYSVLVVAKVE | |
| | KGKSKKLKSVKELLGITIMERSSFE | |
| | KNPIDFLEAKGYKEVKKDLIIKLPK | |
| | YSLFELENGRKRMLASAGELQKGNE | |
| | LALPSKYVNFLYLASHYEKLKGSPE | |
| | DNEQKQLFVEQHKHYLDEIIEQISE | |
| | FSKRVILADANLDKVLSAYNKHRDK | |
| | PIREQAENIIHLFTLTNLGAPAAFK | |
| | YFDTTIDRKRYTSTKEVLDATLIHQ | |
| | SITGLYETRIDLSQLGGD | |
| Cas3 | MKHINDYFWAKKTEENSRLLWLPLT | 18 |
| | QHLEDTKNIAGLLWEHWLSEGQKVL | |
| | IENSINVKSNIENQGKRLAQFLGAV | |
| | HDIGKATPAFQTQKGYANSVDLDIQ | |
| | LLEKLERAGFSGISSLQLASPKKSH | |
| | HSIAGQYLLSHYGVDEDIATIIGGH | |
| | HGRPVDDLDGLNSQKSYPSNYYQDE | |
| | KKDSLVYQKWKSNQEAFLNWALTET | |
| | GFNSVSQLPKIKQPAQVILSGLLIM | |
| | SDWIASNEHFFPLLSLDETDVKNKS | |
| | QRIETGFKKWKKSNLWQPETFVDLV | |
| | TLYQERFGFSPRNFQLILSQTIEKT | |
| | TNPGFVILEAPMGIGKTEAALAVSE | |
| | QLSSKKGCSGLFFGLPTQATSNGIF | |
| | KRIEQWTENIKGNNSDHFSIQLVHG | |
| | KAALNTDFIELLKGNTINMDDSENG | |
| | SIFVNEWFSGRKTSALDDFVVGTVD | |
| | QFLMVALKQKHLALRHLGFSKKVIV | |
| | IDEVHAYDAYMSQYLLEAIRWMGAY | |
| | GVPVIILSATLPAQQREKLIKSYMA | |
| | GMGVKWRDIENIDQIKIDAYPLITY | |
| | NDGPDIHQVKMFEKQEQKNIYIHRL | |
| | PEEQLFDIVKEGLDNGGVVGIIVNT | |
| | VRKSQELARNFSDIFGDDMVDLLHS | |
| | NFIATERIRKEKDLLQEIGKKAIRP | |
| | PKKIIIGTQVLEQSLDIDFDVLISD | |
| | LAPMDLLIQRIGRLHRHKIKRPQKH | |
| | EVARFYVLGTFEEFDFDEGTRLVYG | |
| | DYLLARTQYFLPDKIRLPDDISPLV | |
| | QKVYNSDLTITFPKPELHKKYLDAK | |
| | IEHDDKIKNKETKAKSYRIANPVLK | |
| | KSRVRTNSLIGWLKNLHPNDSEEKA | |
| | YAQVRDIEDTVEVIALKKISDGYGL | |
| | FIENKDISQNITDPIIAKKVAQNTL | |
| | RLPMSLSKAYNIDQTINELERYNNS | |
| | HLSQWQNSSWLKGSLGIIFDKNNEF | |
| | ILNGFKLLYDEKYGVTIERLDKNES | |
| | V | |
| Cas4 | MITEFLLKKKLEEHLSHVKEENTIY | 19 |
| | VTDLVRCPRRVRYESEYKELAISQV | |
| | YAPSAILGDILHLGLESVLKGNFNA | |
| | ETEVETLREINVGGKVYKIKGRADA | |
| | IIRNDNGKSFVIEIKTSRSDKGLPL | |
| | IHHKMQLQIYLWLFSAEKGILVYIT | |
| | PDRIAEYEINEPLDEATIVRLAEDT | |
| | IMLQNSPRFNWECKYCIFSVICPAK | |
| Cas5 | MSPHIDANGVPIDCLSFTVRSTWGH | 20 |
| | FKRVGRSVTKQTYRIPPRTTVAGML | |
| | AAFVGADRDSYYDVFGADNSAIAIT | |
| | PLSDLRTVNIPTTGLGTDPKQDVTE | |
| | TAGSWRTYKLTYQNTTGDRQLHSYE | |
| | VLADPAYRIDVALENEEFYDSLREH | |
| | LVNGTSIYPPSLGKSEYLAVIEDVE | |
| | VDREPEVQDIDETLDIDSVVPISLS | |
| | EAVPQGKVTYGVERSPAVMVRESGG | |
| | RRTTRFDDYVYAQQASNPVRIGEQT | |
| | DVAPVQVGDRTVVFR | |
| PIGA L1 | MGPKKKRKVAAADYKDDDDKSRPGE | 21 |
| | RPFQCRICMRNFSDSPTLRRHTRTH | |
| | TGEKPFQCRICMRNFSQRSSLVRHL | |
| | RTHTGEKPFQCRICMRNFSERGNLT | |
| | RHLKTHLRGSQLVKSELEEKKSELR | |
| | HKLKYVPHEYIELIEIARNSTQDRI | |
| | LEMKVMEFFMKVYGYRGKHLGGSRK | |
| | PDGAIYTVGSPIDYGVIVDTKAYSG | |
| | GYNLPIGQADEMQRYVEENQTRNKH | |
| | INPNEWWKVYPSSVTEFKFLFVSGH | |
| | FKGNYKAQLTRLNHITNCNGAVLSV | |
| | EELLIGGEMIKAGTLTLEEVRRKFN | |
| | NGEINF | |
| PIGA R1 | MGPKKKRKVAAADYKDDDDKSRPGE | 22 |
| | RPFQCRICMRNFSAPSKLKRHTRTH | |
| | TGEKPFQCRICMRNFSHKSSLTRHL | |
| | RTHTGEKPFQCRICMRNFSQRNALA | |
| | GHLKTHLRGSQLVKSELEEKKSELR | |
| | HKLKYVPHEYIELIEIARNSTQDRI | |
| | LEMKVMEFFMKVYGYRGKHLGGSRK | |
| | PDGAIYTVGSPIDYGVIVDTKAYSG | |
| | GYNLPIGQADEMQRYVEENQTRNKH | |
| | INPNEWWKVYPSSVTEFKFLFVSGH | |
| | FKGNYKAQLTRLNHITNCNGAVLSV | |
| | EELLIGGEMIKAGTLTLEEVRRKFN | |
| | NGEINF | |
| AAVS1 | MAPKKKRKVGIHGVPAAMAERPFQC | 23 |
| R1 | RICMRNFSQSSNLARHIRTHTGEKP | |
| | FACDICGRKFARTDYLVDHTKIHTG | |
| | SQKPFQCRICMRNFSYNTHLTRHIR | |
| | THTGEKPFACDICGRKFAQGYNLAG | |
| | HTKIHLRGSQLVKSELEEKKSELRH | |
| | KLKYVPHEYIELIEIARNSTQDRIL | |
| | EMKVMEFFMKVYGYRGKHLGGSRKP | |
| | DGAIYTVGSPIDYGVFVDTKAYSGG | |
| | YNLPIGQADEMQRYVKENQTRNKHI | |
| | NPNEWWKVYPSSVTEFKFLFVSGHF | |
| | KGNYKAQLTRLNHKTNCNGAVLSVE | |
| | ELLIGGEMIKAGTLTLEEVRRKFNN | |
| | GEINF* | |
| AAVS1 | MAPKKKRKVGIHGVPAAMAERPFQC | 24 |
| L1 | RICMRNFSYNWHLQRHIRTHTGEKP | |
| | FACDICGRKFARSDHLTTHTKIHTG | |
| | SQKPFQCRICMRNFSHNYARDCHIR | |
| | THTGEKPFACDICGRKFAQNSTRIG | |
| | HTKIHLRGSQLVKSELEEKKSELRH | |
| | KLKYVPHEYIELIEIARNSTQDRIL | |
| | EMKVMEFFMKVYGYRGKHLGGSRKP | |
| | DGAIYTVGSPIDYGVFVDTKAYSGG | |
| | YNLPIGQADEMERYVEENQTRNKHL | |
| | NPNEWWKVYPSSVTEFKFLFVSGHF | |
| | KGNYKAQLTRLNHITNCNGAVLSVE | |
| | ELLIGGEMIKAGTLTLEEVRRKFNN | |
| | GEINF* | |
| phiC31 | MDTYAGAYDRQSRERENSSAASPAT | 25 |
| Integrase | QRSANEDKAADLQREVERDGGRFRF | |
| | VGHFSEAPGTSAFGTAERPEFERIL | |
| | NECRAGRLNMIIVYDVSRFSRLKVM | |
| | DAIPFVSELLALGVTIVSTQEGVFR | |
| | QGNVMDLIHLIMRLDASHKESSLKS | |
| | AKILDTKNLQRELGGYVGGKAPYGF | |
| | ELVSETKEITRNGRMVNVVINKLAH | |
| | STTPLTGPFEFEPDVIRWWWREIKT | |
| | HKHLPFKPGSQAAIHPGSITGLCKR | |
| | MDADAVPTRGETIGKKTASSAWDPA | |
| | TVMRILRDPRIAGFAAEVIYKKKPD | |
| | GTPTTKIEGYRIQRDPITLRPVELD | |
| | CGPIIEPAEWYELQAWLDGRGRGKG | |
| | LSRGQAILSAMDKLYCECGAVMTSK | |
| | RGEESIKDSYRCRRRKVVDPSAPGQ | |
| | HEGTCNVSMAALDKFVAERIFNKIR | |
| | HAEGDEETLALLWEAARRFGKLTEA | |
| | PEKSGERANLVAERADALNALEELY | |
| | EDRAAGAYDGPVGRKHFRKQQAALT | |
| | LRQQGAEERLAELEAAEAPKLPLDQ | |
| | WFPEDADADPTGPKSWWGRASVDDK | |
| | RVFVGLFVDKTVVTKSTTGRGQGTP | |
| | IEKRASITWAKPPTDDDEDDAQDGT | |
| | EDVAA | |
| I-CreI | MNTKYNKEFLLYLAGFVDGDGSIIA | 26 |
| | QIKPNQSYKFKHQLSLTFQVTQKTQ | |
| | RRWFLDKLVDEIGVGYVRDRGSVSD | |
| | YILSEIKPLHNFLTQLQPFLKLKQK | |
| | QANLVLKIIEQLPSAKESPDKFLEV | |
| | CTWVDQIAALNDSKTRKTTSETVRA | |
| | VLDSLSEKKKSSP | |
| I-SceI | MKNIKKNQVMNLGPNSKLLKEYKSQ | 27 |
| | LIELNIEQFEAGIGLILGDAYIRSR | |
| | DEGKTYCMQFEWKNKAYMDHVCLLY | |
| | DQWVLSPPHKKERVNHLGNLVITWG | |
| | AQTFKHQAFNKLANLFIVNNKKTIP | |
| | NNLVENYLTPMSLAYWFMDDGGKWD | |
| | YNKNSTNKSFVLNTQSFTFEEVEYL | |
| | VKGLRNKFQLNCYVKINKNKPIIYI | |
| | DSMSYLIFYNLIKPYLIPQMMYKLP | |
| | NTISSETFLK | |
| RAG1 | MAASFPPTLGLSSAPDEIQHPHIKF | 28 |
| | SEWKFKLFRVRSFEKTPEEAQKEKK | |
| | DSFEGKPSLEQSPAVLDKADGQKPV | |
| | PTQPLLKAHPKFSKKFHDNEKARGK | |
| | AIHQANLRHLCRICGNSFRADEHNR | |
| | RYPVHGPVDGKTLGLLRKKEKRATS | |
| | WPDLIAKVFRIDVKADVDSIHPTEF | |
| | CHNCWSIMHRKFSSAPCEVYFPRNV | |
| | TMEWHPHTPSCDICNTARRGLKRKS | |
| | LQPNLQLSKKLKTVLDQARQARQHK | |
| | RRAQARISSKDVMKKIANCSKIHLS | |
| | TKLLAVDFPEHFVKSISCQICEHIL | |
| | ADPVETNCKHVFCRVCILRCLKVMG | |
| | SYCPSCRYPCFPTDLESPVKSFLSV | |
| | LNSLMVKCPAKECNEEVSLEKYNHH | |
| | ISSHKESKEIFVHINKGGRPRQHLL | |
| | SLTRRAQKHRLRELKLQVKAFADKE | |
| | EGGDVKSVCMTLFLLALRARNEHRQ | |
| | ADELEAIMQGKGSGLQPAVCLAIRV | |
| | NTFLSCSQYHKMYRTVKAITGRQIF | |
| | QPLHALRNAEKVLLPGYHHFEWQPP | |
| | LKNVSSSTDVGIIDGLSGLSSSVDD | |
| | YPVDTIAKRFRYDSALVSALMDMEE | |
| | DILEGMRSQDLDDYLNGPFTVVVKE | |
| | SCDGMGDVSEKHGSGPVVPEKAVRF | |
| | SFTIMKITIAHSSQNVKVFEEAKPN | |
| | SELCCKPLCLMLADESDHETLTAIL | |
| | SPLIAEREAMKSSELMLELGGILRT | |
| | FKFIFRGTGYDEKLVREVEGLEASG | |
| | SVYICTLCDATRLEASQNLVFHSIT | |
| | RSHAENLERYEVWRSNPYHESVEEL | |
| | RDRVKGVSAKPFIETVPSIDALHCD | |
| | IGNAAEFYKIFQLEIGEVYKNPNAS | |
| | KEERKRWQATLDKHLRKKMNLKPIM | |
| | RMNGNFARKLMTKETVDAVCELIPS | |
| | EERHEALRELMDLYLKMKPVWRSSC | |
| | PAKECPESLCQYSFNSQRFAELLST | |
| | KFKYRYEGKITNYFHKTLAHVPEII | |
| | ERDGSIGAWASEGNESGNKLFRRFR | |
| | KMNARQSKCYEMEDVLKHHWLYTSK | |
| | YLQKFMNAHNALKTSGFTMNPQASL | |
| | GDPLGIEDSLESQDSMEF | |
| Sleeping | MGKSKEISQDLRKRIVDLHKSGSSL | 29 |
| Beauty | GAISKRLAVPRSSVQTIVRKYKHHG | |
| Transposon | TTQPSYRSGRRRVLSPRDERTLVRK | |
| (SB100X) | VQINPRTTAKDLVKMLEETGTKVSI | |
| | STVKRVLYRHNLKGHSARKKPLLQN | |
| | RHKKARLRFATAHGDKDRTFWRNVL | |
| | WSDETKIELFGHNDHRYVWRKKGEA | |
| | CKPKNTIPTVKHGGGSIMLWGCFAA | |
| | GGTGALHKID | |
| TALEN | MSRTRLPSPPAPSPAFSADSFSDLL | 30 |
| | RQFDPSLFNTSLFDSLPPFGAHHTE | |
| | AATGEWDEVQSGLRAADAPPPTMRV | |
| | AVTAARPPRAKPAPRRRAAQPSDAS | |
| | PAAQVDLRTLGYSQQQQEKIKPKVR | |
| | STVAQHHEALVGHGFTHAHFVALSQ | |
| | HPAALGTVAVKYQDMIAALPEATHE | |
| | AIVGVGKQWSGARALEALLTVAGEL | |
| | RGPPLQLDTGQLLKIAKRGGVTAVE | |
| | AVHAWRNALTGAPLNLTETVHETHG | |
| | TASQLTPEQVVAIASNIGGRPALES | |
| | IVAQLSRPDPALAALTNDHLVALAC | |
| | LGGRPALDAVKKGLPHAPALIKRTN | |
| | RRIPERTSHRVADHAQVVRVLGFFQ | |
| | CHSHPAQAFDDAMTQFGMSRHGLLQ | |
| | LFRRVGVTELEARSGTLPPASQRWD | |
| | RILQASGMKRAKPSPTSTQTPDQAS | |
| | LHAFADSLERDLDAPSPMHEGDQTR | |
| | ASASPKKKRKVEASGSGRADALDDF | |
| | DLDMLGSDALDDFDLDMLGSDALDD | |
| | FDLDMLGSDALDDFDLDMLIN | |

In some embodiments, the plasmid for transfection comprises a nucleic acid sequence encoding a Cas9 protein. Cas9 is an RNA-guided DNA endonuclease that cleaves a DNA substrate having sequence complementary to the guide RNA. The guide RNA has a constant part and a variable part. The variable part contains 20 nucleotides, the sequence of which will guide the Cas9 protein to the specific site in the genome to create a double strand break. Thus, in some embodiments, the plasmid for transfection further comprises a guide RNA comprising a variable part having a nucleic acid sequence that is complementary to a portion of a target gene to be modified. In some embodiments, more than one 20-nucleotide variable parts, such as 2, 3, 4, 5, or more, are used for a single target gene.

In some embodiments, the plasmid for transfection further comprises a marker gene to facilitate the selection of transfected keratinocytes. Any marker gene known in the art can be used. Examples of marker genes include, but not limited to, fluorescent reporter genes, antibiotic resistance genes and surface protein-coding genes. Functional fragments of the proteins encoded by such marker genes, for instance, functional fragments having at least about 75%, 80%, 85%, 90%, 91%, 92%, 93%, 94%, 95%, 96%, 97%, 98%, 99% or more sequence identity to the parent protein can also be used. Proteins encoded by representative marker genes are provided in Table 3. Functional fragments of these proteins encoded by the representative marker genes therefore may comprise an amino acid sequence having at least about 75%, 80%, 85%, 90%, 91%, 92%, 93%, 94%, 95%, 96%, 97%, 98%, 99% or more sequence identity to any of SEQ ID NO: 317, SEQ ID NO: 318, SEQ ID NO: 319, SEQ ID NO: 320, SEQ ID NO: 321, SEQ ID NO: 322, SEQ ID NO: 323.

TABLE 3
Representatives of marker genes.

| | | SEQ |
|---|---|---|
| Name | Sequence | ID NO |
| GFP | VSKGEELFTGVVPILVELDGDVNGHKFSVSGEGEGDATYGKLTLKFICTTGK | 317 |
| | LPVPWPTLVTTLTYGVQCFSRYPDHMKQHDFFKSAMPEGYVQERTIFFKDD | |
| | GNYKTRAEVKFEGDTLVNRIELKGIDFKEDGNILGHKLEYNYNSHNVYIMA | |
| | DKQKNGIKVNFKIRHNIEDGSVQLADHYQQNTPIGDGPVLLPDNHYLSTQSA | |
| | LSKDPNEKRDHMVLLEFVTAAGITLGMDELYK | |
| T dtomato | MVSKGEEVIKEFMRFKVRMEGSMNGHEFEIEGEGEGRPYEGTQTAKLKVTK | 318 |
| | GGPLPFAWDILSPQFMYGSKAYVKHPADIPDYKKLSFPEGFKWERVMNFED | |
| | GGLVTVTQDSSLQDGTLIYKVKMRGTNFPPDGPVMQKKTMGWEASTERLY | |
| | PRDGVLKGEIHQALKLKDGGHYLVEFKTIYMAKKPVQLPGYYYVDTKLDIT | |
| | SHNEDYTIVEQYERSEGRHHLFLGHGTGSTGSGSSGTASSEDNNMAVIKEFM | |
| | RFKVRMEGSMNGHEFEIEGEGEGRPYEGTQTAKLKVTKGGPLPFAWDILSP | |
| | QFMYGSKAYVKHPADIPDYKKLSFPEGFKWERVMNFEDGGLVTVTQDSSL | |
| | QDGTLIYKVKMRGTNFPPDGPVMQKKTMGWEASTERLYPRDGVLKGEIHQ | |
| | ALKLKDGGHYLVEFKTIYMAKKPVQLPGYYYVDTKLDITSHNEDYTIVEQY | |
| | ERSEGRHHLFLYGMDELYK | |
| EBFP2 | MVSKGEELFTGVVPILVELDGDVNGHKFSVRGEGEGDATNGKLTLKFICTT | 319 |
| | GKLPVPWPTLVTTLSHGVQCFARYPDHMKQHDFFKSAMPEGYVQERTIFFK | |
| | DDGTYKTRAEVKFEGDTLVNRIELKGVDFKEDGNILGHKLEYNFNSHNIYI | |
| | MAVKQKNGIKVNFKIRHNVEDGSVQLADHYQQNTPIGDGPVLLPDSHYLST | |
| | QSVLSKDPNEKRDHMVLLEFRTAAGITLGMDELYK | |
| mKate2 | MVSELIKENMHMKLYMEGTVNNHHFKCTSEGEGKPYEGTQTMRIKAVEGG | 320 |
| | PLPFAFDILATSFMYGSKTFINHTQGIPDFFKQSFPEGFTWERVTTYEDGGVL | |
| | TATQDTSLQDGCLIYNVKIRGVNFPSNGPVMQKKTLGWEASTETLYPADGG | |
| | LEGRAD MALKLVGGGHLICNLKTTYRSKKPAKNLKMPGVYYVDRRLERIK | |
| | EADKETYVEQHEVAVARYCDLPSKLGH | |
| mKO | MVSVIKPEMKMRYYMDGSVNGHEFTIEGEGTGRPYEGHQEMTLRVTMAK | 321 |
| | GGPMPFAFDLVSHVFCYGHRPFTKYPEEIPDYFKQAFPEGLSWERSLEFEDG | |
| | GSASVSAHISLRGNTFYHKSKFTGVNFPADGPIMQNQSVDWEPSTEKITASD | |
| | GVLKGDVTMYLKLEGGGNHKCQFKTTYKAAKKILKMPGSHYISHRLVRKT | |
| | EGNITELVEDAVAHS | |
| Puromycin | MTEYKPTVRLATRDDVPRAVRTLAAAFADYPATRHTVDPDRHIERVTELQE | 322 |
| | LFLTRVGLDIGKVWVADDGAAVAVWTTPESVEAGAVFAEIGPRMAELSGS | |
| | RLAAQQQMEGLLAPHRPKEPAWFLATVGVSPDHQGKGLGSAVVLPGVEAA | |
| | ERAGVPAFLETSAPRNLPFYERLGFTVTADVEVPEGPRTWCMTRKPGA | |
| DeltaLNG | MDEKTTGWRGGHVVEGLAGELEQLRARLEHHPQGQREPGSGAIAKEACPT | 323 |
| FR + SBP | GLYTHSGECCKACNLGEGVAQPCGANQTVCEPCLDSVTFSDVVSATEPCKP | |
| (Streptavidin | CTECVGLQSMSAPCVEADDAVCRCAYGYYQDETTGRCEACRVCEAGSGLV | |
| binding | FSCQDKQNTVCEECPDGTYSDEANHVDPCLPCTVCEDTERQLRECTRWAD | |
| peptide) | AECEEIPGRWITRSTPPEGSDSTAPSTQEPEAPPEQDLIASTVAGVVTTVMGS | |
| | SQPVVTRGTTDNLIPVYCSILAAVVVGLVAYIAFKR | |

In some embodiments, the marker gene comprised in the plasmid for transfection encodes a fluorescent reporter of any color, such as green, red, blue or yellow. In some embodiments, the marker gene encodes a green fluorescent protein (GFP). Keratinocytes transfected with a plasmid containing the GFP marker gene generally show GFP fluorescence levels within few hours, such as about 6, 10, 12, 16, 20, 24 or more hours. GFP fluorescence intensity, or other type of fluorescence intensity, can be determined and measured by any methods known in the art. In some embodiments, transfected keratinocytes are selected by Fluorescence-Activated Cell Sorting (FACS). The transfected keratinocytes can be sorted onto tissue culture-treated polystyrene multi-well plates and expanded. Expansion of primary keratinocytes, such as primary human keratinocytes, can be done using any methods known in the art. Thus, in some embodiments, the present disclosure also relates to a method of generating a population of one or a plurality of genome-edited keratinocytes comprising:
a) generating and selecting one or a plurality of genome-edited primary keratinocytes deficient in expression of one or a plurality of wild-type genes according to any method disclosed herein; and
b) expanding the one or plurality of genome-edited primary keratinocytes into the population of one or plurality of genome-edited keratinocytes.
   A schematic of this genome-editing by CRISPR/cas9 transfection followed by FACS selection and expansion according to the present disclosure is shown in FIG. 3.

This step of selecting transfected keratinocytes, however, is notessential to the methods disclosed herein. In some embodiments therefore, the disclosure also relates to a method of generating a population of one or a plurality of genome-edited keratinocytes comprising:
a) generating one or a plurality of genome-edited primary keratinocytes deficient in expression of one or a plurality of wild-type genes according to any method disclosed herein; and
b) expanding the one or plurality of genome-edited primary keratinocytes into the population of one or plurality of genome-edited keratinocytes.

Genome-editing in transfected keratinocytes can be checked by any methods known in the art. One example of such methods is TIDE for Tracking of Indels by Decomposition (Brinkman et al., Easy quantitative assessment of genome editing by sequence trace decomposition, Nucleic Acids Res., 2014 Dec., 16; 42(22):e168; accessible at deskgen.com/landing/tide.html#/tide). The TIDE method, which requires only a pair of PCR reactions and two standard capillary sequencing runs, can deconvolute Sanger capillary sequencing reactions to infer the proportion of a population of cells whose genome has been edited. In some embodiments, any method according to the present disclosure for preparing genome-edited keratinocytes can further comprise a step of checking genome editing by using, for example, the TIDE method.

Compositions and Systems

The disclosure relates to a composition comprising one or a plurality of keratinocytes comprising at least three deficient or mutated genes that regulate cell growth. In some embodiments, the mutated genes are deficient in expression of NOTCH1, CDKN2A and TP53. In some embodiments, the cells are primary cells from a mammal. In some embodiments, the cells are primary human cells. In some embodiments, the cells comprise at least one point mutation in each of the NOTCH1, CDKN2A and TP53 genes. In some embodiments, the cell comprise genome-edited mutations in only the NOTCH1, CDKN2A and TP53 genes. In some embodiments, the mutated genes are deficient in expression of NOTCH1, CDKN2A, TP53, and at least one other gene identified in Table 1.

The genome-edited keratinocytes according to the present disclosure can be used in a composition, which can comprise one or more additional components. In some embodiments, such compositions include a solid support or culture dish comprising a well. In some embodiments, the well further comprise a top layer of cells and a bottom layer of tissue, the bottom layer of tissue comprising extracellular matrix protein and the top layer of cells comprising one or a plurality of genome-edited keratinocytes or those cells from the aforementioned paragraph according to the present disclosure. The disclosure relates to a cell culture system comprising a solid support comprising at least one vessel or insert, Any tissue comprising extracellular matrix protein, including but not limited to dermis and cadaver skin, can be used. In some embodiments, the bottom layer of tissue comprises one or a plurality of fibroblasts. In some embodiments, the compositions of the present disclosure further comprise a hydrogel layer positioned underneath the bottom layer of tissue. In some embodiments, the hydrogel layer comprises one or a plurality of growth factors or functional fragments thereof. Suitable growth factors include, but not limited to the growth factors provided in Table 4.

TABLE 4
List of growth factors.

| Growth | | SEQ |
|---|---|---|
| Factor | Sequence | ID NO |
| EGF | MLLTLIILLPVVSKFSFVSLSAPQHWSCPEGTLAGNGNSTCVGPAPFLIFSHGNS | 340 |
| | IFRIDTEGTNYEQLVVDAGVSVIMDFHYNEKRIYWVDLERQLLQRVFLNGSRQ | |
| | ERVCNIEKNVSGMAINWINEEVIWSNQQEGIITVTDMKGNNSHILLSALKYPA | |
| | NVAVDPVERFIFWSSEVAGSLYRADLDGVGVKALLETSEKITAVSLDVLDKRL | |
| | FWIQYNREGSNSLICSCDYDGGSVHISKHPTQHNLFAMSLFGDRIFYSTWKMK | |
| | TIWIANKHTGKDMVRINLHSSFVPLGELKVVHPLAQPKAEDDTWEPEQKLCK | |
| | LRKGNCSSTVCGQDLQSHLCMCAEGYALSRDRKYCEDVNECAFWNHGCTLG | |
| | CKNTPGSYYCTCPVGFVLLPDGKRCHQLVSCPRNVSECSHDCVLTSEGPLCFC | |
| | PEGSVLERDGKTCSGCSSPDNGGCSQLCVPLSPVSWECDCFPGYDLQLDEKSC | |
| | AASGPQPFLLFANSQDIRHMHFDGTDYGTLLSQQMGMVYALDHDPVENKIYF | |
| | AHTALKWIERANMDGSQRERLIEEGVDVPEGLAVDWIGRRFYWTDRGKSLIG | |
| | RSDLNGKRSKIITKENISQPRGIAVHPMAKRLFWTDTGINPRIESSSLQGLGRLV | |
| | IASSDLIWPSGITIDFLTDKLYWCDAKQSVIEMANLDGSKRRRLTQNDVGHPF | |
| | AVAVFEDYVWFSDWAMPSVMRVNKRTGKDRVRLQGSMLKPSSLVVVHPLA | |
| | KPGADPCLYQNGGCEHICKKRLGTAWCSCREGFMKASDGKTCLALDGHQLL | |
| | AGGEVDLKNQVTPLDILSKTRVSEDNITESQHMLVAEIMVSDQDDCAPVGCS | |
| | MYARCISEGEDATCQCLKGFAGDGKLCSDIDECEMGVPVCPPASSKCINTEGG | |
| | YVCRCSEGYQGDGIHCLDIDECQLGEHSCGENASCTNTEGGYTCMCAGRLSE | |
| | PGLICPDSTPPPHLREDDHHYSVRNSDSECPLSHDGYCLHDGVCMYIEALDKY | |
| | ACNCWGYIGERCQYRDLKWWELRHAGHGQQQKVIWAVCVWLVMLLLL | |
| | SLWGAHYYRTQKLLSKNPKNPYEESSRDVRSRRPADTEDGMSSCPQPWFVVI | |
| | KEHQDLKNGGQPVAGEDGQAADGSMQPTSWRQEPQLCGMGTEQGCWIPVSS | |
| | DKGSCPQVMERSFHMPSYGTQTLEGGVEKPHSLLSANPLWQQRALDPPHQME | |
| | LTQ | |
| bFGF | MVGVGGGDVEDVTPRPGGCQISGRGARGCNGIPGAAAWEAALPRRRPRRHPS | 341 |
| | VNPRSRAAGSPRTRGRRTEERPSGSRLGDRGRGRALPGGRLGGRGRGRAPER | |
| | VGGRGRGRGTAAPRAAPAARGSRPGPAGTMAAGSITTLPALPEDGGSGAFPP | |
| | GHFKDPKRLYCKNGGFFLRIHPDGRVDGVREKSDPHIKLQLQAEERGVVSIKG | |
| | VCANRYLAMKEDGRLLASKCVTDECFFFERLESNNYNTYRSRKYTSWYVAL | |
| | KRTGQYKLGSKTGPGQKAILFLPMSAKS | |
| NGF | MSMLFYTLITAFLIGIQAEPHSESNVPAGHTIPQAHWTKLQHSLDTALRRARSA | 342 |
| | PAAAIAARVAGQTRNITVDPRLFKKRRLRSPRVLFSTQPPREAADTQDLDFEV | |
| | GGAAPFNRTHRSKRSSSHPIFHRGEFSVCDSVSVWVGDKTTATDIKGKEVMVL | |
| | GEVNINNSVFKQYFFETKCRDPNPVDSGCRGIDSKHWNSYCTTTHTFVKALT | |
| | MDGKQAAWRFIRIDTACVCVLSRKAVRRA | |
| PDGF | MNRCWALFLSLCCYLRLVSAEGDPIPEELYEMLSDHSIRSFDDLQRLLHGDPG | 343 |
| | EEDGAELDLNMTRSHSGGELESLARGRRSLGSLTIAEPAMIAECKTRTEVFEIS | |
| | RRLIDRTNANFLVWPPCVEVQRCSGCCNNRNVQCRPTQVQLRPVQVRKIEIVR | |
| | KKPIFKKATVTLEDHLACKCETVAAARPVTRSPGGSQEQRAKTPQTRVTIRTV | |
| | RVRRPPKGKHRKFKHTHDKTALKETLGA | |
| IGF-1 | MGKISSLPTQLFKCCFCDFLKVKMHTMSSSHLFYLALCLLTFTSSATAGPETLC | 344 |
| | GAELVDALQFVCGDRGFYFNKPTGYGSSSRRAPQTGFVDECCFRSCDLRRLEM | |
| | YCAPLKPAKSARSVRAQRHTDMPKTQKYQPPSTNKNTKSQRRKGWPKTHPG | |
| | GEQKEGTEASLQIRGKKKEQRREIGSRNAECRGKKGK | |
| TGF-β | MPPSGLRLLLLLLPLLWLLVLTPGRPAAGLSTCKTIDMELVKRKRIEAIRGQILS | 345 |
| | KLRLASPPSQGEVPPGPLPEAVLALYNSTRDRVAGESAEPEPEPEADYYAKEV | |
| | TRVLMVETHNEIYDKFKQSTHSIYMFFNTSELREAVPEPVLLSRAELRLLRLKL | |
| | KVEQHVELYQKYSNNSWRYLSNRLLAPSDSPEWLSFDVTGVVRQWLSRGGEI | |
| | EGFRLSAHCSCDSRDNTLQVDINGFTTGRRGDLATIHGMNRPFLLLMATPLER | |
| | AQHLQSSRHRRALDTNYCFSSTEKNCCVRQLYIDFRKDLGWKWIHEPKGYHA | |
| | NFCLGPCPYIWSLDTQYSKVLALYNQHNPGASAAPCCVPQALEPLPIVYYVGR | |
| | KPKVEQLSNMIVRSCKCS | |

In some embodiments, the hydrogel layer comprises one or a plurality of growth factors chosen from Table 4 or a functional fragment thereof comprising at least about 70%, 75%, 80%, 85%, 90%, 91%, 92%, 93%, 94%, 95%, 96%, 97%, 98%, 99%, or 100% sequence identity to the amino acid sequence of the growth factors listed in Table 4. The hydrogel used in the compositions of the present disclosure can be those that are commercially available, such as Matrigel® and Cultrex®. Matrigel® is the trade name for a gelatinous protein mixture secreted by Engelbreth-Holm-Swarm mouse sarcoma cells produced and marketed by Corning Life Sciences and BD Biosciences. The base components of Matrigel® are 60% laminin, 30% collagen IV, and 8% entactin. In some embodiments, the Matrigel further comprises the following growth factors:

| Growth Factor | Concentration |
|---|---|
| EGF | From about 0.5 to about |
| | 1.3 ng/mL |
| bFGF | From about 0.01-0.2 |
| | pg/mL |
| NGF | From about 0.01 ng/mL to |
| | about 0.2 ng/mL |
| PDGF | From about 5 pg mL to |
| | about 48 pg/mL |
| IGF-1 | From about 11 ng/mL to |
| | about 24 ng/mL |
| TGF-β | From about 1.7 ng/mL to |
| | about 4.7 ng/mL |

Cultrex® is the trade name for a soluble form of basement membrane purified from Engelbreth-Holm-Swarm (EHS) tumor and marketed by Trevigen, Inc. In some embodiments, the hydrogel used in the compositions of the present disclosure is Matrigel®. In some embodiments, the compositions of the present disclosure is at about 30° C., 35° C., or 37° C. and exposed to about 3%, 4% or 5% CO2 for no less than about 24 or 48 hours.

In some embodiments, the compositions of the present disclosure further comprise at least a single insert, (e.g. portable plastic well with a flat surface whose surface area is defined by one or a plurality of sidewalls, the insert or well comprising a biocompatible polymer and an inner wall defining an inner volume divided into an upper chamber and a bottom chamber, the upper chamber and bottom chamber positioned adjacently to one another and in fluid communication and separated by a ledge; the ledge positioned along an inner wall of the insert and defining a planar interface between the upper and bottom chambers. In some embodiments, the compositions of the present disclosure comprise a top layer of cells, a bottom layer of tissue and a hydrogel; each of the top layer of cells and bottom layer of tissue positioned in the upper chamber; and wherein the hydrogel is positioned in the bottom chamber; wherein the bottom layer of tissue comprises extracellular matrix protein and the top layer of cells comprises one or a plurality of genome-edited keratinocytes keratinocytes according to the present disclosure. The compositions of the present disclosure can comprise more than one insert, such as 2, 6, 12, 24, 48, 96, 192 or more inserts. Thus, in some embodiments, the compositions of the present disclosure relates to a solid support comprising at least about 2, 6, 12, 24, 48, 96, 192 or more inserts. In some embodiments, the inserts are equally sized and positioned in a pattern at discrete positions on or within the solid support.

In some embodiments, the interface of the compositions of the present disclosure is a planar or substantially planar opening with a surface area from about 0.01 to about 20, about 0.05 to about 10, from about 0.1 to about 5, about 0.2 to about 3 square centimeters. In some embodiments, the upper chamber of the compositions of the present disclosure has a volume from about 0.5 to about 20, about 0.3 to about 15, about 0.2 to about 10, about 0.1 to about 9, about 0.05 to about 5 centimeters cubed. In some embodiments, the bottom chamber of the compositions of the present disclosure has a volume from about 0.5 to about 20, about 0.3 to about 15, about 0.2 to about 10, about 0.1 to about 9, about 0.05 to about 5 centimeters cubed. In some embodiments, the compositions of the present disclosure are frozen at from about −20° C. to about −212° C.

The genome-edited keratinocytes according to the present disclosure can be used in a system comprising:
a) at least one solid support comprising a bottom surface and sidewalls, the bottom surface and sidewalls defining a culture volume;
b) at least one insert as described herein positioned on the bottom surface of the at least one solid support; and
c) one or a plurality of genome-edited keratinocytes according to the present disclosure.

In some embodiments, the systems of the present disclosure further comprise a top layer of cells and a bottom layer of tissue, the bottom layer of tissue comprising extracellular matrix protein and the top layer of cells comprising the one or plurality of keratinocytes. In some embodiments, the bottom layer of tissue comprises one or a plurality of fibroblasts.

In some embodiments, the systems of the present disclosure further comprise a hydrogel layer as described herein positioned adjacently underneath the bottom layer of tissue. In some embodiments, the hydrogel is Matrigel®.

In some embodiments, the systems of the present disclosure further comprise an incubator in which the solid support is positioned and wherein the incubator comprises a heating element and an air inlet through which air content is controlled. The incubator maintains the temperature of the systems at about 30° C., 35° C., or 37° C. and comprises ambient air in contact with the solid support that comprises about 3%, 4% or 5% CO2. The systems of the present disclosure can further comprise culture media in the volume of the solid support with a cell culture media volume sufficient to cover the solid support and expose one or plurality of keratinocytes contained therein to air. In some embodiments, the system of the present disclosure further comprises a tissue culture medium at a volume sufficient to cover the bottom tissue and create an air liquid interface immediately adjacent to or below the top layer of cells, such that the top layer of cells is exposed to air. In some embodiments, the air is filtered or sterilized in a closed system.

The solid support of the systems of the present disclosure can be from about 1 to about 10, about 2 to about 8, or about 3 to about 5 centimeters in height.

In some embodiments, the systems of the present disclosure comprise a top layer of cells, a bottom layer of tissue and a hydrogel; each of the top layer of cells and bottom layer of tissue positioned in the upper chamber; and wherein the hydrogel positioned in the bottom chamber; wherein the bottom layer of tissue comprises extracellular matrix protein and the top layer of cells comprises the one or plurality of primary keratinocytes. The systems of the present disclosure can comprise more than one insert, such as 2, 6, 12, 24, 48, 96, 192 or more inserts. Thus, in some embodiments, the systems of the present disclosure comprise at least about 2, 6, 12, 24, 48, 96, 192 or more inserts.

In some embodiments, the interface of the systems of the present disclosure is a planar or substantially planar opening with a surface area from about 0.01 to about 20, about 0.05 to about 10, from about 0.1 to about 5, about 0.2 to about 3 square centimeters. In some embodiments, the upper chamber of the systems of the present disclosure has a volume from about 0.5 to about 20, about 0.3 to about 15, about 0.2 to about 10, about 0.1 to about 9, about 0.05 to about 5 centimeters cubed. In some embodiments, the bottom chamber of the systems of the present disclosure has a volume from about 0.5 to about 20, about 0.3 to about 15, about 0.2 to about 10, about 0.1 to about 9, about 0.05 to about 5 centimeters cubed. In some embodiments, the systems of the present disclosure further comprise a tissue culture medium at a volume sufficient to cover the top layer of cells.

The systems of the present disclosure can further comprise a gas reservoir in fluid communication with the solid support via one or a plurality of gas lines; and a heating element operably positioned at or proximate to the solid support, such that the temperature of ambient air around the top layer of cells is from about 32° C. to about 42°.

The systems of the present disclosure can further comprise one or more therapeutic compounds. The therapeutic can be a nucleic acid molecule, a peptide or a small molecule, or any combination thereof

Methods

In one aspect, the present disclosure related to a method of isolating a primary keratinocytes from skin tissue or mucosa comprising culturing a primary keratinocyte to culture medium while simultaneously exposing one or more keratinocytes to one or a plurality of tissue samples comprising extracellular matrix (ECM). The skin tissue can be obtained from any part of a human body, such as forehead, face, neck, head, or leg, but not limited thereto. The skin tissue can also be anal skin. The mucosa can be mucosa of any source, such as oral mucosa, mucosa of the upper aerodigestive tract, esophageal mucosa, vaginal mucosa, or cervical mucosa, but not limited thereto.

The present disclosure further relates to a method of culturing a primary keratinocyte comprising exposing the primary keratinocyte to a system comprising:
a) at least one solid support comprising a bottom surface and sidewalls, the bottom surface and sidewalls defining a culture volume; and
b) at least one insert as described herein positioned on the bottom surface of the at least one solid support; wherein the insert comprises a layer of tissue comprising ECM onto which the primary keratinocytes are positioned.

The step of exposing can further comprise exposing the primary keratinocyte to cell culture media comprising:

a) DMEM/HAM F12(3:1) supplemented with 10% FBS;

b) 1% penicillin/streptomycin;

c) 1% antibiotic-antimycotic;

d) 3 μg/mL of adenine;

e) 3 ng/mL of cholera toxin;

f) 40 ng/mL of hydrocortisone;

g) 5 μg/mL of insulin;

h) 10 ng/mL of EGF;

i) 5 μg/mL of Transferrin;

j) 1.5 ng/mL of triiodo-L-thyronine; and

k) 10 ng/mL of ciprofloxacin hydrochloride,

for a time period sufficient to propagate the one or plurality of primary keratinocytes for no less than about one, two, three, four, five, six, seven, eight, nine or ten days. The step of exposing can further comprise positioning a layer of hydrogel as described herein underneath the layer of tissue comprising ECM. In some embodiments the time period sufficient to propagate the one or plurality of primary keratinocytes further comprises exposing th cells to about 5% oxygen in air and culturing the cells at about 37 degrees Celsius.

In some embodiments, the disclosure relates to a method of manufacturing a system or culture of primary keratinocytes using any one or more method steps disclosed herein and further comprising fixing the tissue in culture. In some embodiments, fixation can be achieved by exposing the tissue to ethanol or formalin. In some embodiments, cultures may be embedded in paraffin or embedded in OCT.

The method can further comprise a step of exposing the one or plurality of primary keratinocytes to one or a plurality of therapeutics or agents. The therapeutic can be a nucleic acid molecule, a peptide or a small molecule, or any combination thereof. A step of monitoring morphology of the one or plurality of primary keratinocytes in response to exposure to the one or plurality of therapeutics can also be included in the method.

The disclosure relates to a method of altering the genetic material of a primary keratinocyte comprising exposing the primary keratinocyte to one or a plurality of gene modifying enzymes, such as a Cas9 protein, that are specific for NOTCH1, CDKN2A and TP53. In some embodiments, the cells are primary cells from a mammal. In some embodiments, the cells are primary human cells. In some embodiments, the cells comprise at least one point mutation in each of the NOTCH1, CDKN2A and TP53 genes. In some embodiments, the exposing step results in the cells comprising genome-edited mutations in only the NOTCH1, CDKN2A and TP53 genes. In some embodiments, the mutated genes are deficient in expression of NOTCH1, CDKN2A, TP53, and at least one other gene identified in Table 1. In some embodiments the method of altering the primary keratinocyte comprises the step of isolating the one or plurality of keratinocytes prior to the exposure step. In some embodiments, the method of altering the primary keratinocyte comprises the step of culturing the primary keratinocytes with any of the conditions or duration of time disclosed herein.

The culture systems used in such methods can comprise from about 1 to about 200, from about 5 to about 150, from about 10 to about 100, or from about 20 to about 50 inserts.

Any composition described herein according to the present disclosure can be used in a method of screening a compound for biological activity. Thus, the present disclosure also relates to a method of screening a compound for biological activity comprising exposing one or a plurality of compounds to one or a plurality of genome-edited keratinocytes according to the present disclosure. The compounds can be nucleic acid molecules, peptides, or small molecules, or any combination thereof.

### EXAMPLES

### Example 1: Construction of Vectors

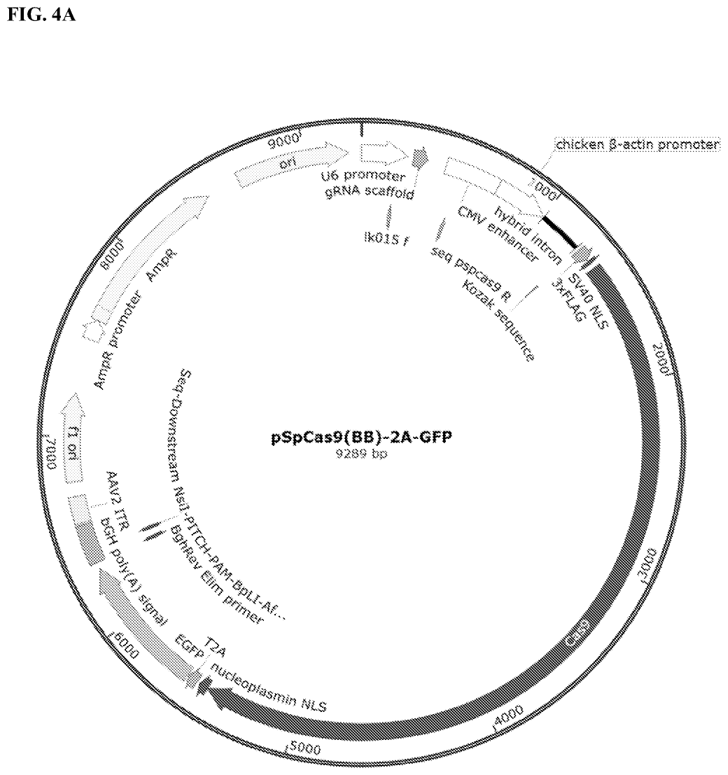

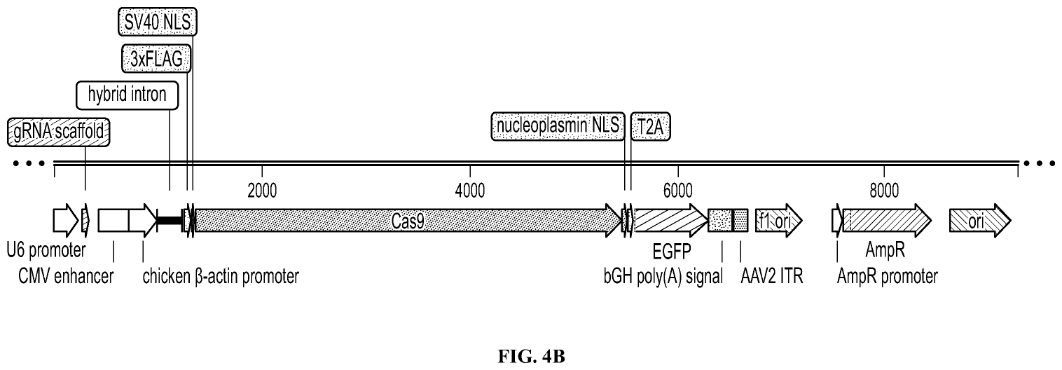

The plasmid pSpCas9(BB)-2A-GFP (Ran et al., “Genome engineering using the CRISPR-Cas9 system,” Nat. Protoc., 2013 November; 8(11):2281-308) was used to construct vectors for transfection. The plasmid comprises the following components: pUC (origin of replication; SEQ ID NO: 1), U6 promoter (guide RNA promoter; SEQ ID NO: 2), CMV promoter (Cas9-GFP promoter; SEQ ID NO: 3), Cas9 gene (SEQ ID NO: 4); GFP gene (SEQ ID NO: 5), Ampicillin promoter (SEQ ID NO; 6), Ampicillin (Bacteria selection marker; SEQ ID NO: 7), and guide RNA scaffold (SEQ ID NO: 8). The full pSpCas9(BB)-2A-GFP vector nucleotide sequence is provided in SEQ ID NO: 9. A schematic of the plasmid is shown in FIG. 4A (vector map) and 4B (linearized). This plasmid encodes for both the cas9 protein and the guide RNA(gRNA). The gRNA has a constant part (depicted in FIG. 4A and FIG. 4B as “gRNA scaffold” in the schematic) and a variable part. The variable part is the 20-nucleotide sequence that starts right at the end of the gRNA scaffold. This 20-nucleotide sequence is the one that will guide the cas9 protein to the specific site in the genome where it will create a double strand break. The 20-nucleotide sequences for each of the genes that were targeted are provided in the column entitled “Small guide RNA (5′>3′)” of Table 5.

The plasmid pSpCas9(BB)-2A-GFP was also modified to include different selection markers, including different fluorescent markers (e.g., EBFP2, Cerulean, Citrine, mKO, dTomato, and mkate2), different antibiotic resistance genes (e.g., puramycin), and different surface markers (e.g., SBP-ALNGFR).

TABLE 5
Small guide RNas and primers used for genome-editing and genome-editing
assessment.

| Gene | Small guide RNA (5′ > 3′) | TIDE primers (5′ > 3′) | |
|---|---|---|---|
| TP53 | 1: CCATTGTTCAATATCGTCCG | F: ACTGACCGTGCAAGTCACAG | SEQ ID NO: 35 |
| | SEQ ID NO: 31 | R: TGGGTTGTGGTGAAACATTG | SEQ ID NO: 36 |
| | 2: GAGCGCTGCTCAGATAGCGA | F: CTCGATCTCCCAACCTCGTGA | SEQ ID NO: 37 |
| | SEQ ID NO: 32 | R: GAGGTGTAGACGCCAACTCTCTCT | SEQ ID NO: 38 |
| | 3: GATCCACTCACAGTTTCCAT | F: TTCTGGGAAGGGACAGAAGA | SEQ ID NO: 39 |
| | SEQ ID NO: 33 | R: GGAAGGTTGGAAGTCCCTCT | SEQ ID NO: 40 |
| | 4: GGTGCCCTATGAGCCGCCTG | F: CTCGATCTCCCAACCTCGTGA | SEQ ID NO: 41 |
| | SEQ ID NO: 34 | R: GAGGTGTAGACGCCAACTCTCTCT | SEQ ID NO: 42 |
| NOTCH1 | 1: TCCTGCCAGAACACCCACGG | F: CATCCTCGGCTCAGTGAAGA | SEQ ID NO: 47 |
| | SEQ ID NO: 43 | R: AATCCGAGGACTATGAGAGCTT | SEQ ID NO: 48 |
| | 2: TCGCACGCCTCCTCGATCAG | F: GAGCAGAGCCTTAGAACTGCAT | SEQ ID NO: 49 |
| | SEQ ID NO: 44 | R: GTGTGTAAAGGGTTTTGCTGCT | SEQ ID NO: 50 |
| | 3: TGCAGGTCAGTACTGTACCG | F: GGGCATCCCGTGACACTTG | SEQ ID NO: 51 |
| | SEQ ID NO: 45 | R: CAGTGGGGTGTCCTGGCATT | SEQ ID NO: 52 |
| | 4: TTGACGTCGATCTCGCATCG | F: GGACTTGGGACAGAAACGAA | SEQ ID NO: 53 |
| | SEQ ID NO: 46 | R: CTGTAACGAGGGCTCCAACT | SEQ ID NO: 54 |
| CDKN2A | 1: CACCGAATAGTTACGGTCGG | F: GACTCCCTTTTTATCCCAAACG | SEQ ID NO: 59 |
| | SEQ ID NO: 55 | R: CCAGTCCTCCTTCCTTGCCAAC | SEQ ID NO: 60 |
| | 2: CGGGTCGGGTGAGAGTGGCG | F: CGGGCTGAACTTTCTGTGCT | SEQ ID NO: 61 |
| | SEQ ID NO: 56 | R: GGCCTAACATAATTTCAGGAAGA | SEQ ID NO: 62 |
| | 3: GATGATGGGCAGCGCCCGAG | F: CGGGCTGAACTTTCTGTGCT | SEQ ID NO: 63 |
| | SEQ ID NO: 57 | R: GGCCTAACATAATTTCAGGAAGA | SEQ ID NO: 64 |
| | 4: GTGGCCAGCCAGTCAGCCGA | F: GACTCCCTTTTTATCCCAAACG | SEQ ID NO: 65 |
| | SEQ ID NO: 58 | R: CCAGTCCTCCTTCCTTGCCAAC | SEQ ID NO: 66 |
| TP63 | 1: CAATGATTAAAATTGGACGG | F: ATCCCCAGTTTTCCCTTTTC | SEQ ID NO: 71 |
| | SEQ ID NO: 67 | R: AGCCAGTGGATGTCTCAAGG | SEQ ID NO: 72 |
| | 2: GCAGTCGAGCACCGCCAAGT | F: CCGTTACAAACACACACATGC | SEQ ID NO: 73 |
| | SEQ ID NO: 68 | R: CGTTCACGTTCTGGAGACTG | SEQ ID NO: 74 |
| | 3: GCTGAGCCGTGAATTCAACG | F: GCAGCATGCAGCTCTAAAAA | SEQ ID NO: 75 |
| | SEQ ID NO: 69 | R: ACACCATTCTCCTGCCTCAG | SEQ ID NO: 76 |
| | 4: TGTGTGTTCTGACGAAACGC | F: TCCCTACCATCCTTCCACCT | SEQ ID NO: 77 |
| | SEQ ID NO: 70 | R: TCCCCATTATGGACTGAAGG | SEQ ID NO: 78 |
| FAT1 | 1: AATTCCCAGAGATCTAGGCG | F: CCAGTCGGAAGAGTGCTTCT | SEQ ID NO: 83 |
| | SEQ ID NO: 79 | R: CAAATAGCAATCCCCCTGAA | SEQ ID NO: 84 |
| | 2: ACTTGATCGAGAAACCCCGG | F: ATTTCCGGTGGAAGGATTTT | SEQ ID NO: 85 |
| | SEQ ID NO: 80 | R: TCCATTCTGTCTGGCAATGA | SEQ ID NO: 86 |
| | 3: CTAACCGTTAGAGCTTCCGA | F: TGCAGAAGGCTTATGTTCCTC | SEQ ID NO: 87 |
| | SEQ ID NO: 81 | R: CGAGCAGCTGATGCTGATAA | SEQ ID NO: 88 |
| | 4: CTACGACAGTCACTTCGATG | F: AACGTTGGTTTGGTTTGGTT | SEQ ID NO: 89 |
| | SEQ ID NO: 82 | R: AAGCTTTTAAATGGCCCAGTG | SEQ ID NO: 90 |
| CASP8 | 1: AGGGGACTCGGAGACTGCGA | F: TGCACGTCCATGAATTGTCT | SEQ ID NO: 95 |
| | SEQ ID NO: 91 | R: CAGGGCCAAATGAAAAAGAA | SEQ ID NO: 96 |
| | 2: CTACCTAAACACTAGAAAGG | F: CCCATTGGCTTGTTTGTATGTC | SEQ ID NO: 97 |
| | SEQ ID NO: 92 | R: AAGCCTCCCAAGTGATACTGAA | SEQ ID NO: 98 |
| | 3: GCCTGGACTACATTCCGCAA | F: CCCATTGGCTTGTTTGTATGTC | SEQ ID NO: 99 |
| | SEQ ID NO: 93 | R: AAGCCTCCCAAGTGATACTGAA | SEQ ID NO: 100 |
| | 4: TCTACTGTGCAGTCATCGTG | F: CAATCGCAGGCACTCGGCA | SEQ ID NO: 101 |
| | SEQ ID NO: 94 | R: GGATGTACCAGGTTCCCTCTGCAG | SEQ ID NO: 102 |
| RB1 | 1: AAACAATCAAAGGACCGAGA | F: TGGCTTTGTTGACCAAACTG | SEQ ID NO: 107 |
| | SEQ ID NO: 103 | R: TGCCATTCTGACTCTTGGAA | SEQ ID NO: 108 |
| | 2: AACATCTAATGGACTTCCAG | F: CACAGATTTTTTACTGCATGGG | SEQ ID NO: 109 |
| | SEQ ID NO: 104 | R: GATGGCATTAATCATGGTTCC | SEQ ID NO: 110 |
| | 3: GGTTCTTTGACAACATGGG | F: TCATACTTCAAAATCTTTCTCTGGA | SEQ ID NO: 111 |
| | SEQ ID NO: 105 | R: ATGCCTTTCCCCAATCCTAA | SEQ ID NO: 112 |
| | 4: TGAACTACTTACGAACTGCT | F: GCCTCAAAAGACCACTCAGG | SEQ ID NO: 113 |
| | SEQ ID NO: 106 | R: TGGAGCTCTACATGGGCATA | SEQ ID NO: 114 |
| PTEN | 1: AGAGCGTGCAGATAATGACA | F: TCATGTGAATGAAAATGCAACA | SEQ ID NO: 119 |
| | SEQ ID NO: 115 | R: GAAGGCAGACAGGAGTGGAA | SEQ ID NO: 120 |
| | 2: AGCTGGCAGACCACAAACTG | F: TTTCGTGACTACTCCCTGTTGA | SEQ ID NO: 121 |
| | SEQ ID NO: 116 | R: ATGCCATAAGGCCTTTTCCT | SEQ ID NO: 122 |
| | 3: ATTCTTCATACCAGGACCAG | F: TCATGTGAATGAAAATGCAACA | SEQ ID NO: 123 |
| | SEQ ID NO: 117 | R: GAAGGCAGACAGGAGTGGAA | SEQ ID NO: 124 |
| | 4: CCAATTCAGGACCCACACGA | F: TTTCGTGACTACTCCCTGTTGA | SEQ ID NO: 125 |
| | SEQ ID NO: 118 | R: ATGCCATAAGGCCTTTTCCT | SEQ ID NO: 126 |
| NSD1 | 1: AAGCACATAAAGATGAACGG | F: GGAAGCCAGTGTTGGACTTG | SEQ ID NO: 131 |
| | SEQ ID NO: 127 | R: TTGTGGCAGCAAACCTAGAA | SEQ ID NO: 132 |
| | 2: GAATTGCTAGTTAAAACGCC | F: GGTTCATCCACTTTTTGTAGCC | SEQ ID NO: 133 |
| | SEQ ID NO: 128 | R: CCCAAGTAACACAAGGATTGG | SEQ ID NO: 134 |
| | 3: GCCCTATCGGCAGTACTACG | F: TTTTTGTTGCTGTGGGCTTA | SEQ ID NO: 135 |
| | SEQ ID NO: 129 | R: ACATGGCGAAATCCTGTCTC | SEQ ID NO: 136 |
| | 4: TATGCATGATAGTAAGACGA | F: TGCAGTTTGAAATGCTGCTC | SEQ ID NO: 137 |
| | SEQ ID NO: 130 | R: AATGAACCTCCACGTTCTCG | SEQ ID NO: 138 |
| KMT2D | 1: CCCCTATCCTAGTCCTACGG | F: GGCCCATAGCTAGGAGAGGA | SEQ ID NO: 143 |
| | SEQ ID NO: 139 | R: CAACAAGGTGCAGAAGGTGA | SEQ ID NO: 144 |
| | 2: CCTTCGAAGCGAAAGTACTG | F: GGGCCCTCCAGTTTAATTTC | SEQ ID NO: 145 |
| | SEQ ID NO: 140 | R: GTGCTGGCATTCAGCAAGTA | SEQ ID NO: 146 |
| | 3: GGTGGAAATTCCCGCCAACG | F: GATCCTCACAGGGCAACTTC | SEQ ID NO: 147 |
| | SEQ ID NO: 141 | R: CCAAGGGGGATGGATTTTAT | SEQ ID NO: 148 |
| | 4: TTCGGGGTAGACCTCCATAG | F: TAGCCCCTCACCTGTTTGAT | SEQ ID NO: 149 |
| | SEQ ID NO: 142 | R: CTTCCCAGTGCTCTCCTCCT | SEQ ID NO: 150 |
| AJUBA | 1: AAATCGGGGGGCAACGACTG | F: GTCGTAGCCCATGCTGATG | SEQ ID NO: 155 |
| | SEQ ID NO: 151 | R: GGCTGCAGGGAAAGAGAAC | SEQ ID NO: 156 |
| | 2: ACCCGACGGCTCAATCCCCG | F: CATCCCTCTTGCTTCTTTCCTA | SEQ ID NO: 157 |
| | SEQ ID NO: 152 | R: GCTCTAGCTTCGCCAGTAGCTC | SEQ ID NO: 158 |
| | 3: TAGGTCCCCCCAACCCACTT | F: GTCGTAGCCCATGCTGATG | SEQ ID NO: 159 |
| | SEQ ID NO: 153 | R: GAACAGATAGACCTGCGGACTG | SEQ ID NO: 160 |
| | 4: TGGACCGATTGTACGCTCAG | F: CATCCCTCTTGCTTCTTTCCTA | SEQ ID NO: 161 |
| | SEQ ID NO: 154 | R: GCTCTAGCTTCGCCAGTAGCTC | SEQ ID NO: 162 |
| EP300 | 1: ATGGTGAACCATAAGGATTG | F: TACCGATGGGTTTTCACCAT | SEQ ID NO: 167 |
| | SEQ ID NO: 163 | R: TCCTGTTTGGATCAACATGC | SEQ ID NO: 168 |
| | 2: CTGTAATAAGTGGCATCACG | F: TTGCTGTGTTGCCTAAGCTG | SEQ ID NO: 169 |
| | SEQ ID NO: 164 | R: CCAAAAAGCTGTCTAGCGAGT | SEQ ID NO: 170 |
| | 3: GGTACGACTAGGTACAGGCG | F: TTCTGTGACATAGCAATGGTGTT | SEQ ID NO: 171 |
| | SEQ ID NO: 165 | R: TGCTGTGCTCTGCTGTGAG | SEQ ID NO: 172 |
| | 4: GTGGCACGAAGATATTACTC | F: TCTGGGATTGCACCACTACA | SEQ ID NO: 173 |
| | SEQ ID NO: 166 | R: TGAGCTGAGGCCTAGTTTTTC | SEQ ID NO: 174 |
| EPHA2 | 1: CACACACCCGTATGGCAAAG | F: AAGACAGGGGGCATCTTTAAC | SEQ ID NO: 179 |
| | SEQ ID NO: 175 | R: GCGGTGTGGTGTCACTTAATAC | SEQ ID NO: 180 |
| | 2: CTGGTGCGGGTCAGTCCGTG | F: TTGCTCTAGACACTGTGCCTTT | SEQ ID NO: 181 |
| | SEQ ID NO: 176 | R: CTCCTGCGAGTGTGAGGAA | SEQ ID NO: 182 |
| | 3: GAAGCCCCTGAAGACATACG | F: ATGATGTTGTGGTGGCTGAA | SEQ ID NO: 183 |
| | SEQ ID NO: 177 | R: GACCAAAGTAGGGCCAGGAG | SEQ ID NO: 184 |
| | 4: TCACGGAGAAACCCTCGGTG | F: CGGGAGGTATTGCAGGTATG | SEQ ID NO: 185 |
| | SEQ ID NO: 178 | R: CTGCAGATGGGGAAACTGAG | SEQ ID NO: 186 |
| FBXW7 | 1: AAGAGCGGACCTCAGAACCA | F: AACAAGACACAGGGGCTCAT | SEQ ID NO: 191 |
| | SEQ ID NO: 187 | R: CACGAGGAAGGCACGTTAAA | SEQ ID NO: 192 |
| | 2: ACAGAATTGATACTAACTGG | F: CAGAGGAGGCAGCAAATTCT | SEQ ID NO: 193 |
| | SEQ ID NO: 188 | R: TGTGTTTGTTTTGGGGAACC | SEQ ID NO: 194 |
| | 3: ACATTAGTGGGACATACAGG | F: TCAAACAGGAAGCTGACAACA | SEQ ID NO: 195 |
| | SEQ ID NO: 189 | R: TGGTATGTAGCCACACCCAAT | SEQ ID NO: 196 |
| | 4: GTTGGAGTAGAACCTAGACC | F: GGCTCAATTACTTTCCCTCCA | SEQ ID NO: 197 |
| | SEQ ID NO: 190 | R: ATTTTCCCCTGCAGAATGTG | SEQ ID NO: 198 |
| RASA1 | 1: AATTTAAGAACAGATGAACA | F: TTACACCCCAAAACCTTCAA | SEQ ID NO: 203 |
| | SEQ ID NO: 199 | R: GCACGAGAAATGCTTGAACC | SEQ ID NO: 204 |
| | 2: ATCGCCAGGAGTATTATCTG | F: AAGCAAACTGGTCTTCACTTTTT | SEQ ID NO: 205 |
| | SEQ ID NO: 200 | R: TCACAAAAATTGTTTTGGAAGG | SEQ ID NO: 206 |
| | 3: ATTTACGGAAAAGTAGTCCA | F: AATCAGCACTCTCCCCCTCT | SEQ ID NO: 207 |
| | SEQ ID NO: 201 | R: GAACCGTTTCTGCTGTGTTG | SEQ ID NO: 208 |
| | 4: GGATGGACCAGAATACGAGG | F: GTAACAGCCGGAGCTGGAG | SEQ ID NO: 209 |
| | SEQ ID NO: 202 | R: CCTCAACTTGGAAGGGACAG | SEQ ID NO: 210 |
| TGFBR2 | 1: ACAGTGATCACACTCCATGT | F: CATGAACCCACTTCCTGACA | SEQ ID NO: 215 |
| | SEQ ID NO: 211 | R: TCACACAGGCAGCAGGTTAG | SEQ ID NO: 216 |
| | 2: ACCTACAGGAGTACCTGACG | F: CATGAACCCACTTCCTGACA | SEQ ID NO: 217 |
| | SEQ ID NO: 212 | R: TCACACAGGCAGCAGGTTAG | SEQ ID NO: 218 |
| | 3: GCAGAAGCTGAGTTCAACCT | F: CATGAACCCACTTCCTGACA | SEQ ID NO: 219 |
| | SEQ ID NO: 213 | R: TCACACAGGCAGCAGGTTAG | SEQ ID NO: 220 |
| | 4: TATCATGTCGTTATTAACTG | F: ATCCTCTGTGCTGCCCTCTA | SEQ ID NO: 221 |
| | SEQ ID NO: 214 | R: TGCAGAGAACACCCCTAGAAA | SEQ ID NO: 222 |
| ZNF750 | 1: CAAGGGGCCAGACAACGCCG | F: GGCTTCTTCAAGCAGGTGAG | SEQ ID NO: 227 |
| | SEQ ID NO: 223 | R: TTCCAAGTCTGTCGCAAATG | SEQ ID NO: 228 |
| | 2: GCGGGTACTCTGGGATTGTG | F: GGCTTCTTCAAGCAGGTGAG | SEQ ID NO: 229 |
| | SEQ ID NO: 224 | R: TTCCAAGTCTGTCGCAAATG | SEQ ID NO: 230 |
| | 3: GTTCGAAAGTCCAATTCCTG | F: TGGAACCCAACCAGAAACAT | SEQ ID NO: 231 |
| | SEQ ID NO: 225 | R: CCATTTCCCCTTACATGCAC | SEQ ID NO: 232 |
| | 4: TCTGGCCTGTAAAATCCGTA | F: TGGAACCCAACCAGAAACAT | SEQ ID NO: 233 |
| | SEQ ID NO: 226 | R: CCATTTCCCCTTACATGCAC | SEQ ID NO: 234 |
| KEAP1 | 1: ACAACCCCATGACCAATCAG | F: AGGAACCATATGGGTTTGTGAC | SEQ ID NO: 239 |
| | SEQ ID NO: 235 | R: TCAACTGGGTCAAGTACGACTG | SEQ ID NO: 240 |
| | 2: AGTACGACTGCGAACAGCGA | F: GAGCGACTGTCGGAAGTAGCC | SEQ ID NO: 241 |
| | SEQ ID NO: 236 | R: CTTCAGCCTCCCGAGTAGCTG | SEQ ID NO: 242 |
| | 3: CAGCACCGTTCATGACGTGG | F: CCCCTCAAACTGTGGAGACT | SEQ ID NO: 243 |
| | SEQ ID NO: 237 | R: TATCTTCTGGAACCCCATGC | SEQ ID NO: 244 |
| | 4: CCTGGAGGATCATACCAAGC | F: CCAATCTGCTCAGCGAAGTT | SEQ ID NO: 245 |
| | SEQ ID NO: 238 | R: GCCAATCAGGTTGATCAGGT | SEQ ID NO: 246 |
| HRAS | 1: CCAGGCTCACCTCTATAGTG | F: ATGGCAAACACACACAGGAA | SEQ ID NO: 251 |
| | SEQ ID NO: 247 | R: TTTTCCCATCACTGGGTCAT | SEQ ID NO: 252 |
| | 2: GGACTCGGATGACGTGCCCA | F: GACACTCTGGGGACAAGAGG | SEQ ID NO: 253 |
| | SEQ ID NO: 248 | R: GTTGGACATCCTGGATACCG | SEQ ID NO: 254 |
| | 3: TGACCTGGCTGCACGCACTG | F: GACACTCTGGGGACAAGAGG | SEQ ID NO: 255 |
| | SEQ ID NO: 249 | R: GTTGGACATCCTGGATACCG | SEQ ID NO: 256 |
| | 4: TGTTGATGGCAAACACACAC | F: CAAGGGAGAGGGTCAGTGAG | SEQ ID NO: 257 |
| | SEQ ID NO: 250 | R: CCAGGACACAGCCAGGATAG | SEQ ID NO: 258 |
| PIK3CA | 1: AACCTCGAACCATAGGATCT | F: CCCCGAAATTCTACCCAAAT | SEQ ID NO: 263 |
| | SEQ ID NO: 259 | R: AAAAGTGCCCAATCCTTTGA | SEQ ID NO: 264 |
| | 2: GAAGCTGTATAATGCTTGGG | F: GGCCGTGATCCTTGTTTTTA | SEQ ID NO: 265 |
| | SEQ ID NO: 260 | R: AGGTCTTTCTGCCAAACGAA | SEQ ID NO: 266 |
| | 3: GGATTTAGCTATTCCCACGC | F: TGGGGAAGAAAAGTGTTTTGA | SEQ ID NO: 267 |
| | SEQ ID NO: 261 | R: TCCATGTTGTTCAAGGGTCA | SEQ ID NO: 268 |
| | 4: GTTCGAACAGGTATCTACCA | F: ACTGCTTCACCAGCTAAGGA | SEQ ID NO: 269 |
| | SEQ ID NO: 262 | R: CTCAGCCTTCCAGGTTCAAG | SEQ ID NO: 270 |
| RHOA | 1: AAAACACATCAGTATAACAT | F: TGTTGCTCAGTCTGGAATGC | SEQ ID NO: 275 |
| | SEQ ID NO: 271 | R: GGGCGACAGAGCAAGACTAC | SEQ ID NO: 276 |
| | 2: ACAGAAATGCTTGACTTCTG | F: TTGCAGTGAGCTGAGATTGC | SEQ ID NO: 277 |
| | SEQ ID NO: 272 | R: CCCCGTGAAACTACCTTGTG | SEQ ID NO: 278 |
| | 3: CTATGTGGCAGATATCGAGG | F: ACATGCCTGTAATCCCTGCT | SEQ ID NO: 279 |
| | SEQ ID NO: 273 | R: GAACTGAGATTGGGCCAGTG | SEQ ID NO: 280 |
| | 4: GCCACTCACCTAAACTATCA | F: TGTTGCTCAGTCTGGAATGC | SEQ ID NO: 281 |
| | SEQ ID NO: 274 | R: GGGCGACAGAGCAAGACTAC | SEQ ID NO: 282 |
| CCR5 | 1: CAATGTGTCAACTCTTGACA | F: CTCCGCTCTACTCACTGGTGTTC | SEQ ID NO: 287 |
| | SEQ ID NO: 283 | R: GACAAAGGCATAGATGATGGGGTTG | SEQ ID NO: 288 |
| | 2: CATTAAAGATAGTCATCTTG | F: CTCCGCTCTACTCACTGGTGTTC | SEQ ID NO: 289 |
| | SEQ ID NO: 284 | R: GACAAAGGCATAGATGATGGGGTTG | SEQ ID NO: 290 |
| | 3: GGTGACAAGTGTGATCACTT | F: CTCCGCTCTACTCACTGGTGTTC | SEQ ID NO: 291 |
| | SEQ ID NO: 285 | R: GACAAAGGCATAGATGATGGGGTTG | SEQ ID NO: 292 |
| | 4: TCATCCTCCTGACAATCGAT | F: CTCCGCTCTACTCACTGGTGTTC | SEQ ID NO: 293 |
| | SEQ ID NO: 286 | R: GACAAAGGCATAGATGATGGGGTTG | SEQ ID NO: 294 |
| ROSA26 | 1: GTTGCTTTATCACTCAACAG | F: TGCCATCATAGTGAACTACAGCCT | SEQ ID NO: 298 |
| | SEQ ID NO: 295 | R: GGGAGGATGTGTGTAAGTTGTATGC | SEQ ID NO: 299 |
| | 2: GGAATGATCTTGAATAGTGT | F: TGCCATCATAGTGAACTACAGCCT | SEQ ID NO: 300 |
| | SEQ ID NO: 296 | R: GGGAGGATGTGTGTAAGTTGTATGC | SEQ ID NO: 301 |
| | 3: AATGCAGGTATGCCACTACT | F: TGCCATCATAGTGAACTACAGCCT | SEQ ID NO: 302 |
| | SEQ ID NO: 297 | R: GGGAGGATGTGTGTAAGTTGTATGC | SEQ ID NO: 303 |
| AAVS1 | 1: AATGAATTGATGCAGGTCTG | F: GAGCCAAAGTTAGAACTCAGGACC | SEQ ID NO: 305 |
| | SEQ ID NO: 304 | R: ACCTCTCACTCCTTTCATTTGG | SEQ ID NO: 306 |

### Example 2: Isolation and Culture of Primary Cells

Primary adult human epidermal and oral keratinocytes were isolated from fresh surgical specimens. Primary newborn foreskin keratinocytes were acquire from the department of dermatology core service (UCSF). Surgical samples were incubated in dispase (Corning, #354235) overnight, after which epidermis was physically separated from dermis using sterilized tweezers. To dissociate the epidermis, 0.5% trypsin (Life Techonologies, #15400054) and 3 minutes incubation was used. Cells were plated in 6-well plate using keratinocyte media. Keratinocyte media was composed by keratinocyte-SFM (Life technologies, #17005042) and 154 medium (Life techcnologies, #M154500) in a 1:1 ratio, supplemented with human keratinocyte growth supplement (HKGS, Life technologies, #S0015). Primary keratinocytes were grown at 37° C., 5% CO2. Cells were monitored periodically and split before cultures reaching 90% confluency using 0.05% trypsin (Life technologies, #25300054).

Primary adult fibroblast were isolated from the dermis produced in the keratinocyte isolation protocol. Collagenase P (Sigma-Aldrich, #11213857001) and dispase (Corning, #354235) were used to dissociate fibroblasts. Fibroblasts were grown in DMEM (Life Technologies, #12491023) supplemented with 10% fetal bovine serum (FBS, Fisher Scientific, #MT35010CV) and 1× penicillin/streptomycin (Fisher Scientific, #MT30002CI) at 37 C, 5% CO₂.

### Example 3: Primary Cells Transfection

Keratinocytes were seeded on tissue culture-treated polystyrene 6-well plates (Corning, #0877249) at a density of 150,000 cells per well. 3 μg of vector DNA and 4.5 μL of TranslT-X2 (Mirus Bio, #mir6000) was used per transfection. In the case of multiple gene targeting experiments, the total amount of vector DNA (3 μg) was created by combining different vectors encoding for distinct guide RNAs in a 1:1 ratio. Cells were incubated with transfection complexes for 24 hours at 37° C., 5% CO2.

Alternatively, keratinocytes were transfected by electroporatioin using, for example, a Lonza 4D-Nucleofector X Unit (Lonza, #AAF-1002X). To this end, CRISPR/Cas9 ribonucleoprotein (RNP) complexes were formed by mixing crRNA:tracrRNA (50 μM) and Cas9 protein (40 μM) at a ratio 16:10 and subsequently incubated for 15 minutes at room temperature. An example of tracrRNAs may have the sequence (5^{mut} to 3′) of GTTTTAGAGCT AGAAATAGCAAGTTAAAATAAGGCTAGTCCGTTATCAACTTGAAAAAGTGGCACCG AGTCGGTGC (SEQ ID NO: 346). The guide RNAs are comprised in the crRNA molecules used to form CRISPR/Cas9 RNP complexes. TracrRNA molecules were tagged with the fluorescent molecule ATTO 550 (Integrated DNA technologies, #1075928) for transfection efficiency assessment. CRISPR/Cas9 RNPs complexes were added to 500,000 keratinocytes, resuspended in nucleofector solution (Lonza, # V4XP-3024), at a final concentration of 1 μM (final volume 25 μL). RNPs targeting the individual genes were prepared using specific crRNA molecules and, if combined, final concentration for each target was kept at 1 μM. For transfections with plasmid DNA, a total amount of 3 μg per target was added to the cells. Electroporation was carried out using the Lonza 4D-Nucleofector X Unit (Lonza, #AAF-1002X) and suggested protocol for Amaxa™ 4D-Nucleofector™ Protocol for Normal Human Epidermal Keratinocytes (NHEK) kit (Lonza, # V4XP-3024). Cells were pipetted into 6-well plates and kept in the incubator (37° C., 5% CO₂) afterwards.

### Example 4: Primary Cells Selection and Expansion

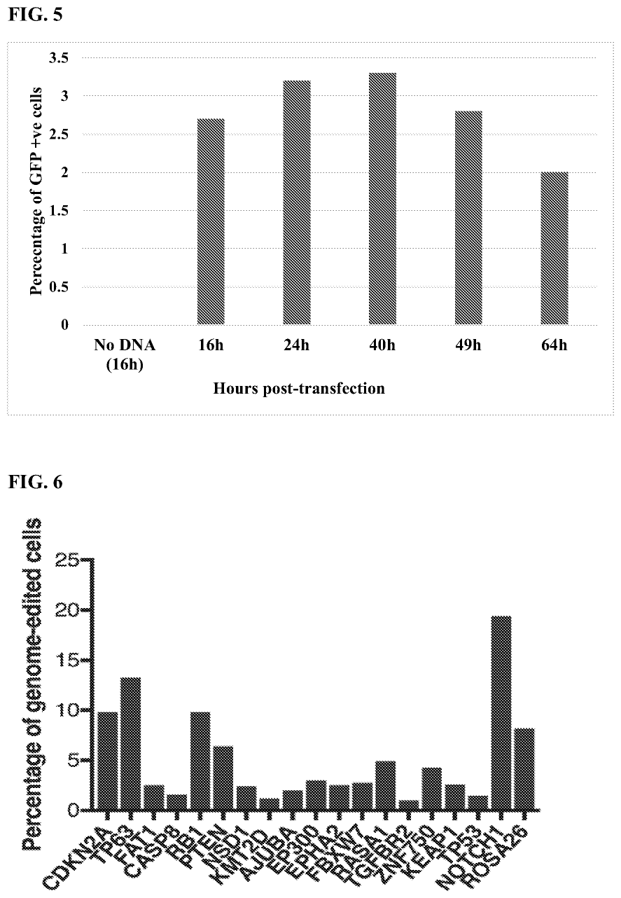

Keratinocytes transfected with pSpCas9(BB)-2A-GFP vectors show GFP fluorescence levels within few hours. FIG. 5 shows the percentage of GFP positive cells detected after up to 64 hours post-transfection. The percentage of GFP positive cells observed at 24 hours post-transfection appeared to be comparable to that observed at 40 hours post-transfection, which is the highest amongst all the time points tested. The 24-hour post-transfection was therefore chosen for cell harvest. FIG. 6 shows the percentage of primary keratinocytes showing genome-editing 24 hours after transfection, which evidences the capacity of the method according to the present disclosure to knock out these genes in primary keratinocytes.

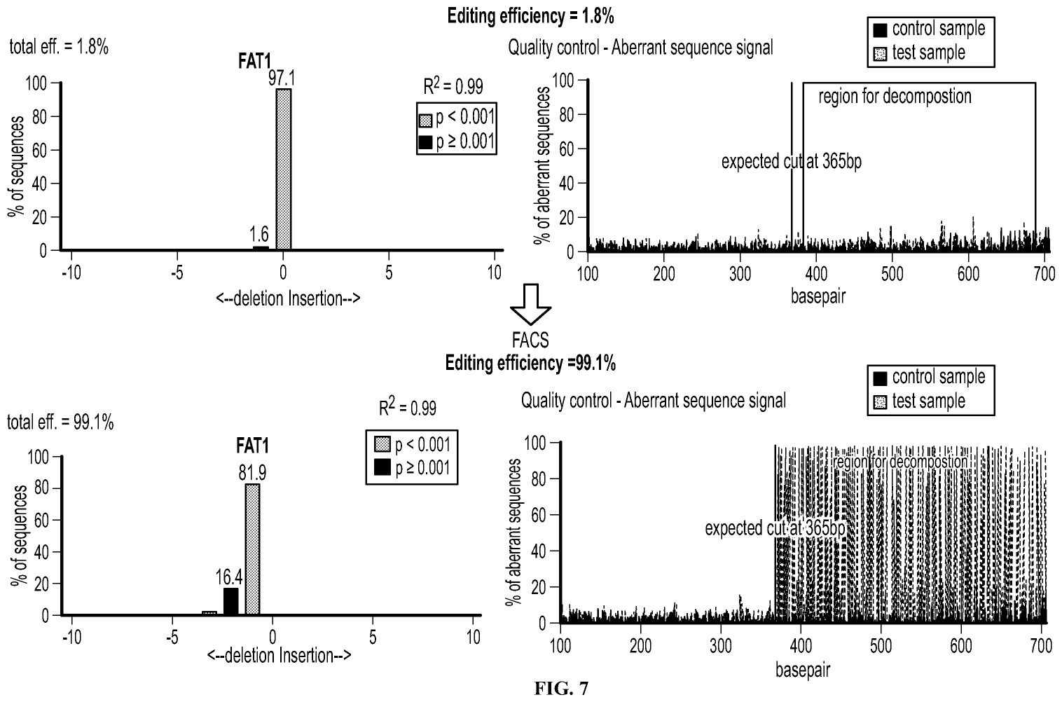

Twenty-four hours after transfection, cells were harvested and dissolved in sorting buffer (5% FBS, 5 mM EDTA (Invitrogen, # AM9261) in PBS (Life Technologies, # AM9624)). Transfected cells were selected by Fluorescence-Activated Cell Sorting (FACS). GFP fluorescence intensity was determined by the SONY SH800 sorter and software. Transfected keratinocytes were sorted onto tissue culture-treated polystyrene 96-well plates (Corning, #CLS3595) and cultures were expanded. Genome-editing in transfected keratinocytes was checked by a method named TIDE for Tracking of Indels by Decomposition (Brinkman et al., Easy quantitative assessment of genome editing by sequence trace decomposition, Nucleic Acids Res., 2014 Dec., 16; 42(22):e168; accessible at deskgen.com/landing/tide.html#/tide). The TIDE method, which requires only a pair of PCR reactions and two standard capillary sequencing runs, can deconvolute Sanger capillary sequencing reactions to infer the proportion of a population of cells whose genome has been edited. The primers for genome-editing assessment using this TIDE method are provided in the column entitled “TIDE primers (5′>3′)” of Table 5. FIG. 7 shows the genome editing efficiency measured by TIDE before and after FACS selection. As shown in FIG. 7, the genome editing efficiency increased from 1.8% to 99.1% by way of the FACS selection.

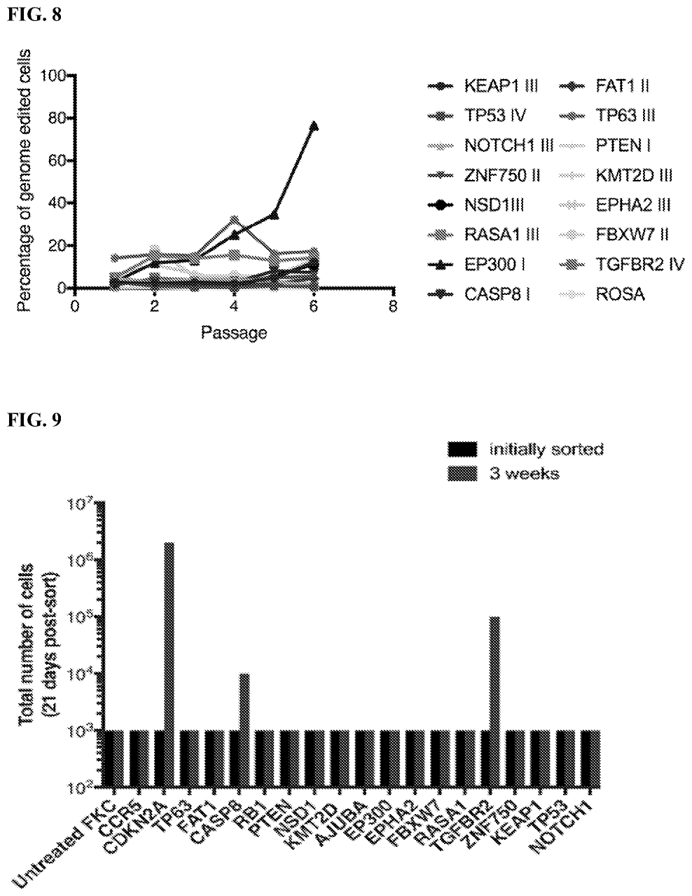

The behavior of genome-edited keratinocytes against untreated (untransfected) cells was also analyzed, the results of which may give a hint of the effect of the individual gene knock-out (KO). FIG. 8 shows the percentage of genome-edited keratinocytes in multiple conditions (in each condition, one single gene was targeted with CRISPR/Cas9) over different passages. In the majority of the conditions, the genome-edited population disappears, while in others, such as KEAP1 KO cells or EP300 KO cells, the KO cells seem to take over the untreated population. In some conditions, such as RASA1, the percentages of edited and unedited population remain constant.

The viability of sorted genome-edited primary keratinocytes (pure populations) in vitro was also analyzed. FIG. 9 shows the recovery of genome-edited keratinocytes harboring one single mutation in the genome following FACS-selection. Among the genes analyzed, only CDKN2A KO cells could be recovered and expanded, while CASP8 KO and TGFBR2 KO cells recovered temporally, but after few doublings these cells become senescent.

### Example 5: Stand Preparation for Three-Dimensional Organotypic Culture

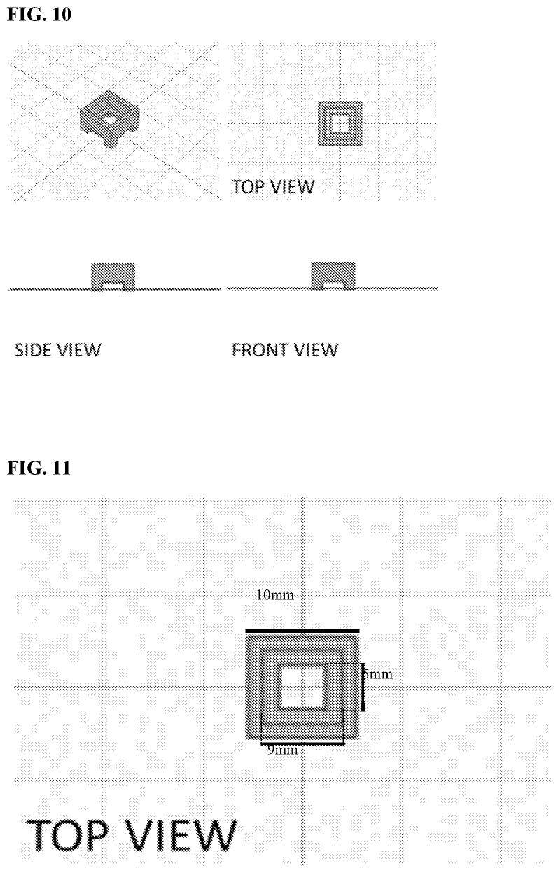

Stands were design using the online tool TINKERCAD. The final design is a 10 by 10 mm hollow cube with walls on the sides, and an internal rim that leaves an opening of 5 by 5 mm on the bottom. 2 mm legs were included on all the bottom corners to increase the volume of media to be used in the organotypic cultures. A schematic representation of a stand design, including the 3D view, top view, side view, and front view, is provided in FIG. 10. FIG. 11 further shows the ratio of ledge surface and cavity area of a stand from a top view. This stand design provides 56 mm² of ledge surface and 25 mm² of cavity area, thus a ledge surface/cavity area ratio of 2.24. Stands were printed with autoclavable ABS plastic using an “Objet Alaris Desktop 3D Printer.” Stands were sterilized using an standard autoclave.

### Example 6: Three-Dimensional Organotypic Culture

Dermis section was obtained from discarded surgical samples and cadaver skin, and sections were frozen at −80° C. Dermis was thawed and pre-sterilized/decellularized in PBS (Life-Technologies, #AM9624) containing 1% penicillin/streptomycin (Fisher-Scientific, #MT30002CI) and 1% antibiotic-antimycotic (Life-Technologies, #15240062). Dermis was sterilized by exposing it to gamma-radiation for 100 minutes in a Cesium Radiator. Dermis can also be sterilized by incubating in 0.1% peracetic acid (PAA) for 30 minutes followed by washing 3 times with PBS for 1 hour at room temperature.

Prior to fibroblast re-impregnation, dermis was sectioned (500 microns or thinner) with a scalpel in 8 by 8 mm pieces, which were air-dried at room temperature for 3 hours in tissue culture-treated 24 well plates (epidermal side facing down). 25,000 human primary fibroblasts in 1 mL of fibroblast media were reintroduced into each of the dermis pieces by centrifugation (1 hour, 200g). Re-impregnated dermis was incubated for 7 days at 37° C., 5% CO2.

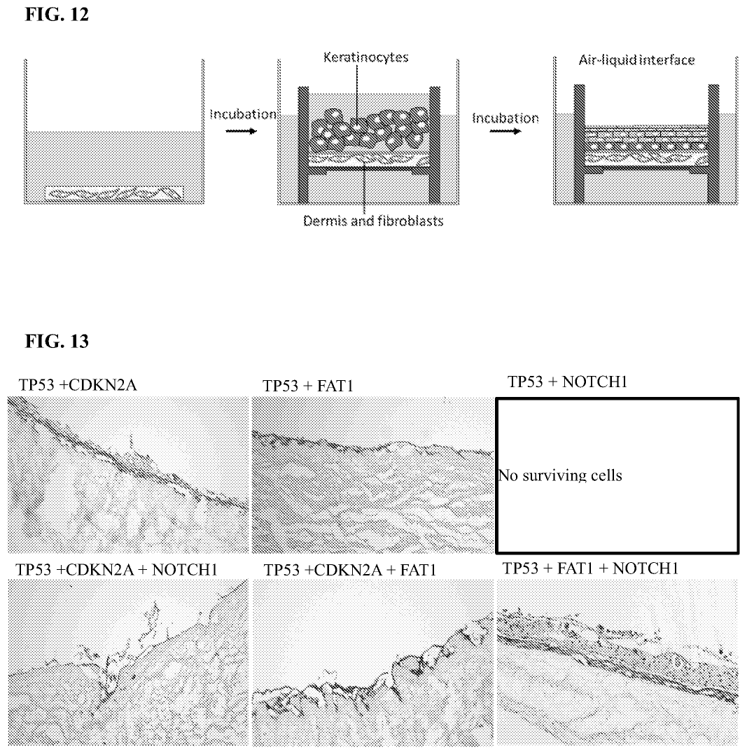

Dermis was flipped and mounted onto 3D-printed stands using sterilized tweezers. A thin layer of matrigel (25-50 μL; Corning, #354235) was applied to the bottom of each of the stands, which were placed in a tissue-culture treated 24 well-plate. 200,000 keratinocytes were resuspended in 50 μL of keratinocyte media and seeded on top of the basal membrane of the dermis. Approximately 500 μL of organotypic culture media was added to the wells in order to create an air-liquid interface. FIG. 12 illustrates this whole process. Organotypic culture media is composed of:
DMEM/HAM F12(3:1; Life Technologies, #12491023; VWR, #12-615F) supplemented with 10% FBS (Fisher Scientific, #MT35010CV)
1% penicillin/streptomycin (Fisher-Scientific, #MT30002CI)
1% antibiotic-antimycotic (Life-Technologies, #15240062)
3 μg/mL of adenine (Sigma, #A9795)
3 ng/mL of cholera toxin (EMD Millipore, #227036)
40 ng/mL of hydrocortisone (EMD Millipore, #386698)
5 μg/mL of insulin (Sigma, #I1882)
10 ng/mL of EGF (Life Technologies, #PHG0315)
5 μg/mL of Transferrin (Sigma, #T0665)
1.5 ng/mL of triiodo-L-thyronine (Sigma, #T2877)
10 ng/mL of ciprofloxacin hydrochloride (Sigma, #PHR1044).
   Organotypic cultures were incubated for 7 days at 37 C, 5% CO2.

Fixation was achieved by immersing the whole culture (including stand) in 4% PFA (Electors microscopy sciences, #15710) overnight. Organotypic cultures was embedded in OCT (Fisher-Scientific, #4585) following incubations in 15%, 30% and 50% sucrose solutions (Fisher Scientific, #50712768). In order to analyze the structure of the organotypic cultures, blocks were sectioned vertically and 5 μm frozen sections were stained using standard H&E or immunofluorescence protocols.

### Example 7: Genome-Edited Keratinocytes on Organotypic Culture

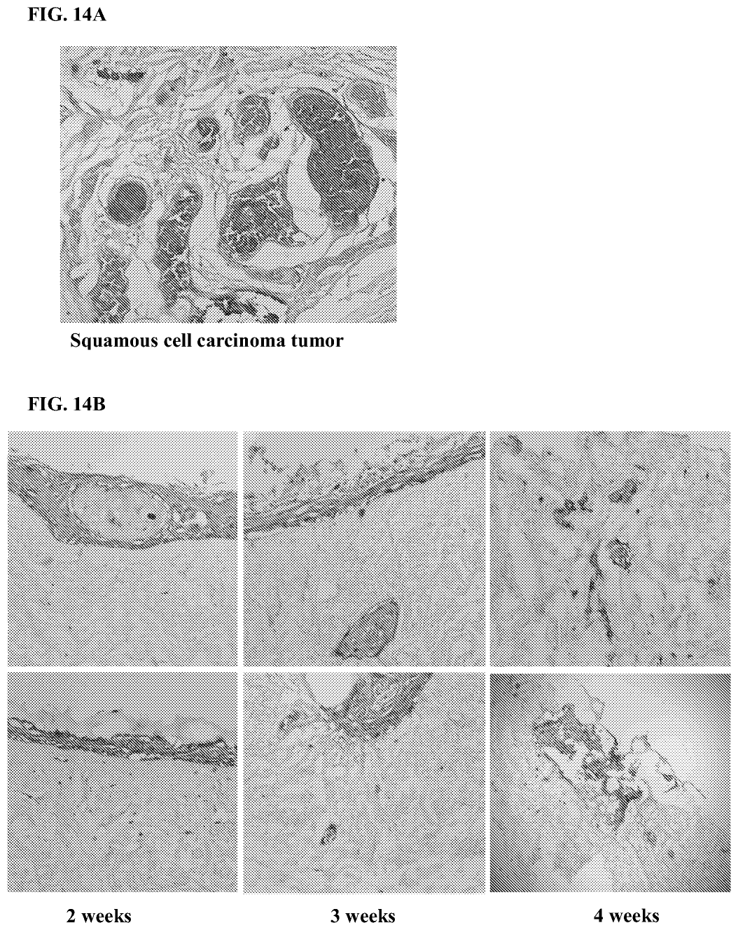

Genome-edited keratinocytes containing single or multiple mutations of the genes listed in Table 1 were generated and tested on organotypic cultures as described above. The structure of the organotypic cultures of these genome-edited keratinocytes were sectioned and stained using standard H&E or immunofluorescence protocols, and the results are shown in FIG. 13. Invasion in keratinocytes containing a combination of TP53, CDKN2A and NOTCH1 mutations were readily detectable within a week. When compared to the stain image obtained from squamous cell carcinoma tumor (FIG. 14A), these TP53+CDKN2A+NOTCH1 mutated keratinocytes appeared to have a similar structure (FIG. 14B). These results indicate that the cells were turned into cancer cells, making it as an authentic genetic model in squamous cell carcinoma.

### Example 8: Minimal Mutational Determinants of Human Squamous Cell Carcinoma

Chronic exposure of squamous epithelia to carcinogenic agents such as UV radiation (1), tobacco smoke, and alcohol (2), or infection by the human papillomavirus (HPV) (3), promotes the development of dysplastic lesions that ultimately progress to squamous cell carcinomas (SCCs). SCCs are among the most prevalent human cancer types and can arise from multiple distinct tissues in the human body (4). It is believed that the sequential accumulation of mutations in relevant epithelial cells is responsible for enabling SCC formation. Although there are promising new therapeutic modalities (5), surgery and radiotherapy with or without classical chemotherapy remain the mainstay of treatment for most types of SCC, and few significant improvements in treatment outcomes have occurred at the population level in the past 40 years (6).

SCC displays a striking commonality of recurrent genomic alterations, despite distinct anatomic sites of origin (7-13). Thus, the molecular signals that govern carcinogenesis are likely shared. In HPV negative head and neck SCC (HNSCC), for example, mutations in the tumor suppressor gene TP53 are found in 85% of the cases. Inactivation of the cyclin-dependent kinase inhibitor CDKN2A occurs in the majority of these cases via point mutation, deletion or methylation-mediated gene silencing (14). Other putative driver mutations have been identified at lower frequencies (14), but validation of their pathogenic role has not been performed in most cases. Despite a high mutation burden (15) and frequent genome instability found in SCC tumors (16), no individual events have been reported to be sufficient or strictly required to trigger SCC. Sequencing studies performed on phenotypically normal skin and esophageal epithelium have shown that apparent driver mutations accumulate in aging keratinocytes without resulting in malignant lesions (17, 18). These mutations resemble those found in SCC, suggesting that specific combinations but not individual genomic alterations are required to transform normal epithelial tissue; however, the specific combinations that are sufficient to do so are currently unknown.

This study is aimed to identify the minimal combination of genetic alterations sufficient for transformation of human keratinocytes by engineering the most common putative driver mutations found in SCC into primary human keratinocytes, in both an individual and combinatorial fashion. It was found that mutations in single driver genes are not sufficient for keratinocyte malignant transformation. In contrast, the combination of TP53 and CDKN2A mutations is sufficient to immortalize keratinocytes and induce dysplastic lesions in organotypic skin and oropharyngeal mucosa cultures. Moreover, keratinocytes harboring mutations in TP53, CDKN2A, and NOTCH1 exhibit an SCC-like phenotype, including invasion through the native dermal basement membrane. Transcriptomic analyses demonstrate that NOTCH1 mutations induce an epithelial to mesenchymal transition (EMT) like phenotype in TP53^{mut} CDKN2A^{mut} human primary keratinocytes while obstructing keratinocyte differentiation.

1. Materials and Methods

i. Isolation, Culture, Genome-Editing and Selection of Primary Keratinocytes

Isolation and culture of primary cells were performed as described in Example 2. Briefly, primary adult human epidermal and oral tonsil keratinocytes were isolated from fresh surgical specimens. Surgical samples were incubated in dispase (Corning, #354235) overnight, after which epidermis was physically separated from dermis using sterilized tweezers. 0.5% trypsin (Life Technologies, #15400054) was used to dissociate the epidermis. Cells were plated in a 6-well plate using keratinocyte media. Keratinocyte media was prepared with keratinocyte-SFM (Life technologies, #17005042) and 154 medium (Life techcnologies, #M154500) in a 1:1 ratio, supplemented with human keratinocyte growth supplement (HKGS, Life Technologies, #S0015). Primary keratinocytes were grown at 37° C., 5% CO2. Cells were monitored periodically and split before cultures reached 90% confluence.

Primary adult fibroblasts were isolated from the dermis produced in the keratinocyte isolation protocol. Collagenase P (Sigma-Aldrich, #11213857001) and dispase (Corning, #354235) were used to dissociate fibroblasts. Fibroblasts were grown in DMEM (Life Technologies, #12491023) supplemented with 10% fetal bovine serum (FBS, Fisher Scientific, #MT35010CV) and 1× penicillin/streptomycin (Fisher Scientific, #MT30002CI) at 37° C., 5% CO2.

The genome of primary keratinocytes was edited via CRISPR/cas9 system (35) as described in Example 1. pSpCas9(BB)-2A-GFP plasmids were modified to include guideRNAs to target different genes (Table 5). Transfection of primary keratinocytes was formed as described in Example 3. Briefly, primary keratinocytes were transfected using 3 μg of DNA plasmids and 4.5 μL of TranslT-X2 (Mints Bio, #mir6000). Incubation for 30 minutes at room temperature prior to treatment was necessary for the formation of transfecting complexes. In the case of multiple gene targeting experiments, the total amount of DNA (3 μg) resulted from the combination of different vectors encoding for distinct guideRNAs in a 1:1 ratio. Cells were incubated with transfection complexes for 24 hours at 37° C., 5% CO2. Ubiquitous genes such as CCR5 and ROSA26 were used as control targets in the genome-editing experiments. A multitude of different guide RNA molecules were developed and used to confirm which specific mutation was responsible for a phenotype. Table 5 contains the list of guideRNAs used in these experiments.

Primary keratinocytes selection and expansion were performed as described in Example 4. Briefly, 24 hours after transfection, keratinocytes were selected by fluorescence-activated cell sorting (FACS) onto 96-well plates (Corning, #CLS3595) using a Sony SH800S cell sorter.

In cell proliferation assays, cells from each condition were seeded in 24-well plates. Cells were counted from three different wells each day, and the plotted value represents the mean. Keratinocytes were stained with DyeCycle Violet (Life technologies, #V35003) following manufacturer's recommendations followed by FACS for cell cycle analysis (10,000 cells per condition). Unpaired t tests (two-tailed) were used in cell proliferation and cell cycle analyses to assess statistical significance.

ii. Genomic DNA Extraction, PCR and Sequencing

Genomic DNA was isolated using the QIAamp Mini Kit (Qiagen, #56304) following manufacturer's instructions. PCR reactions were performed using Q5 High fidelity DNA polymerase (New England Biolabs, #M0491). PCR bands were extracted using NucleoSpin Gel and PCR clean-up columns (Takara, #740609) and sequenced using PCR primers. Genome-editing in transfected keratinocytes was assessed by TIDE (19) as described in Example 4 using the primers for genome-editing assessment provided in the column entitled “TIDE primers (5′>3′)” of Table 5. A diagram representing keratinocyte transfection and sorting based on GFP expression is shown in FIG. 3. TIDE results showing genome-editing efficiency (percentage of cells harboring mutations in the FAT1 gene loci) for non-sorted (top) and sorted (bottom) populations is shown in FIG. 7. Statistical analysis (P-value) associated to each condition was calculated with TIDE (based on two-tailed t-test of the variance-covariance matrix of the standard errors).

Targeted next-generation sequencing of primary keratinocytes was performed using the UCSF500 Cancer Panel as previously described (36, 37). Capture-based next-generation DNA sequencing was performed using an assay that targets a total footprint of ˜2.9 Mb, which includes all coding exons of 479 cancer-related genes, and select introns and upstream regulatory regions of 47 genes to enable detection of TERT promoter variants and structural variants including gene fusions (Table 6). The assay also includes baits for 2,000 unique DNA segments containing common single nucleotide polymorphisms (SNPs) within regions devoid of constitutional copy number variants to enable genome-wide copy number and zygosity analysis. Multiplex library preparation was performed using the KAPA Hyper Prep Kit (Roche, Santa Clara, Calif.) according to the manufacturer's specifications. Hybrid capture of pooled libraries was performed using a custom oligonucleotide library (NimbleGen SeqCap EZ Choice, Roche, Basel, Switzerland). Captured libraries were sequenced as paired-end 100 bp reads on a HiSeq 2500 instrument (Illumina, San Diego, Calif.). Sequence reads were mapped to the reference human genome build GRCh37 (hg19) using the Burrows-Wheeler aligner (bio-bwa.sourceforge.net/). Recalibration and deduplication of reads was performed using the Genome Analysis Toolkit (gatk.broadinstitute.org/hc/en-us). Coverage and sequencing statistics were determined using Picard CalculateHsMetrics and Picard CollectlnsertSizeMetrics. Single nucleotide variant and insertion/deletion mutation calling was performed with Unified Genotyper and Delly. Variant annotation was performed with Annovar. Single nucleotide variants, insertions/deletions, and structural variants were visualized and verified using Integrated Genome Viewer. Genome-wide copy number analysis based on on-target and off-target reads was performed by CNVkit and visualized using Nexus Copy Number (Biodiscovery, El Segundo, Calif.).

TABLE 6
Targeted gene panel-List of the 479 genes targeted for sequencing on the
UCSF500 Cancer Panel. In addition to the coding exons, select intronic or upstream
regulatory regions are also captured for genes highlighted in bold.

| | | | | | | | |
|---|---|---|---|---|---|---|---|
| ABL1 | CD274 | EP300 | GATA2 | KMT2D | NRAS | RAD21 | SPRY2 |
| ABL2 | CDC42 | EPCAM | GATA3 | KNSTRN | NSD1 | RAD50 | SPRY4 |
| ACVR1 | CDC73 | EPHA2 | GLI1 | KRAS | NSD2 | RAD51 | SPTA1 |
| ACVR1B | CDH1 | EPHA3 | GLI2 | LEF1 | NT5C2 | RAD51C | SRC |
| AJUBA | CDK12 | EPHA5 | GNA11 | LIFR | NTRK1 | RAD51D | SRSF2 |
| AKT1 | CDK4 | EPHA7 | GNA13 | LRP1B | NTRK2 | RAF1 | SS18 |
| AKT2 | CDK6 | EPHBI | GNAQ | LZTR1 | NTRK3 | RARA | STAG2 |
| AKT3 | CDK8 | EPOR | GNAS | MALAT1 | NUP93 | RASA1 | STAT3 |
| ALK | CDKN1A | ERBB2 | GPC3 | MAML2 | NUTM1 | RASA2 | STAT4 |
| APC | CDKN1B | ERBB3 | GPR124 | MAP2K1 | OR5L1 | RB1 | STAT6 |
| APOBEC3G | CDKN2A | ERBB4 | GRIN2A | MAP2K2 | PAK1 | RBM10 | STK11 |
| AR | CDKN2B | ERCC1 | GRM3 | MAP2K4 | PAK3 | REL | SUFU |
| ARAF | CDKN2C | ERCC2 | GSK3B | MAP3K1 | PALB2 | RELA | SYK |
| ARFRP1 | CEBPA | ERG | H3F3A | MAP3K2 | PARK2 | RET | SYNE1 |
| ARHGAP35 | CHD1 | ERRFI1 | H3F3B | MAP3K5 | PAX3 | RHEB | TADA1 |
| ARID1A | CHD2 | ESPL1 | HDAC4 | MAP3K7 | PAX5 | RHOA | TBX3 |
| ARID1B | CHD4 | ESR1 | HDAC9 | MAP3K9 | PAX7 | RICTOR | TCEB1 |
| ARID2 | CHD5 | ESR2 | HEY1 | MAPK1 | PAX8 | RIT1 | TCF7L2 |
| ARID5B | CHEK1 | ETS1 | HGF | MCL1 | PBRM1 | RNF43 | TERT |
| ASH2L | CHEK2 | ETV6 | HIF1A | MDM2 | PDCD1LG2 | ROBO1 | TET2 |
| ASXL1 | CIC | EWSR1 | HIST1H3B | MDM4 | PDGFB | ROS1 | TFE3 |
| ASXL2 | CLDN18 | EZH1 | HMGA2 | MED12 | PDGFRA | RPL10 | TFEB |
| ATF1 | CNOT3 | EZH2 | HNF1A | MEF2B | PDGFRB | RPTOR | TGFBR2 |
| ATM | COL1A1 | FAM123B | HOXB13 | MEN1 | PDK1 | RRAGC | TLR4 |
| ATR | COL2A1 | FAM46C | HRAS | MET | PHF6 | RRAS | TNFAIP3 |
| ATRX | CRCT1 | FANCA | HSPA2 | MGA | PHOX2B | RRAS2 | TNFRSF14 |
| AURKA | CREB1 | FANCC | HSPA5 | MGMT | PIK3CA | RSPO2 | TOP1 |
| AURKB | CREBBP | FANCE | HSP90AB1 | MITF | PIK3CG | RSPO3 | TOP2A |
| AXIN1 | CRKL | FANCF | ID3 | MLH1 | PIK3R1 | RUNX1 | TMPRSS2 |
| AXIN2 | CSF1R | FANCG | IDH1 | MLH3 | PIK3R2 | RUNX1T1 | TP53 |
| AXL | CSF3R | FANCL | IDH2 | MPL | PLAG1 | SDHB | TRAF3 |
| BAP1 | CTCF | FAT1 | IGF1R | MRE11A | PLCB4 | SDHD | TRAF7 |
| BARD1 | CTNNA1 | FAT3 | IGF2 | MSH2 | PMS1 | SETBP1 | TRIM28 |
| BCL2 | CTNNB1 | FBXW7 | IGF2R | MSH3 | POLD1 | SETD2 | TSC1 |
| BCL2A1 | CUL3 | FGF10 | IKBKE | MSH6 | POLE | SF3B1 | TSC2 |
| BCL2L1 | CUX1 | FGF14 | IKZF1 | MTOR | POLQ | SH2B3 | TSHR |
| BCL2L12 | CYLD | FGF19 | IKZF2 | MUTYH | POT1 | SHH | TSHZ2 |
| BCL2L2 | CXCR4 | FGF23 | IKZF3 | MYB | POU3F2 | SIN3A | TSHZ3 |
| BCL6 | DCC | FGF3 | IL2RB | MYBL1 | PPM1D | SLIT2 | TSLP |
| BCOR | DDIT3 | FGF4 | IL7R | MYC | PPP2R1A | SLITRK6 | TTYH1 |
| BCORL1 | DDR2 | FGF6 | INHBA | MYCL1 | PPP6C | SMAD2 | TYK2 |
| BLM | DDX3X | FGFR1 | INPP4B | MYCN | PRDM1 | SMAD3 | U2AF1 |
| BRAF | DDX41 | FGFR2 | IPMK | MYD88 | PREX2 | SMAD4 | USP7 |
| BRCA1 | DGKH | FGFR3 | IRF4 | MYH9 | PRKACA | SMARCA2 | VEGFA |
| BRCA2 | DICER1 | FGFR4 | IRS2 | NAV3 | PRKAG2 | SMARCA4 | VHL |
| BRD4 | DIS3 | FH | JAK1 | NBN | PRKAR1A | SMARCB1 | WISP3 |
| BRIP1 | DNAJB1 | FLCN | JAK2 | NCKAP5 | PRKCA | SMC1A | WRN |
| BTG1 | DNMT3A | FLT1 | JAK3 | NCOA2 | PRKCH | SMC3 | WT1 |
| BTK | DOT1L | FLT3 | JAZF1 | NCOA3 | PRKDC | SMO | XBP1 |
| CALR | DUSP2 | FLT4 | KAT6A | NCOR1 | PTCH1 | SNCAIP | XPO1 |
| CARD11 | DUSP4 | FOXA1 | KDM5A | NF1 | PTCH2 | SOCS1 | YAP1 |
| CBFB | DUSP6 | FOXL2 | KDM5C | NF2 | PTEN | SOS1 | YWHAE |
| CBL | DYNC1I1 | FOXO1 | KDM6A | NFE2L2 | PTK2B | SOS2 | ZBTB20 |
| CBLB | EBF1 | FOXP1 | KDR | NFKBIA | PTPN1 | SOX9 | ZFHX3 |
| CCND1 | EDNRB | FRS2 | KEAP1 | NFKBIE | PTPN11 | SOX10 | ZMYM3 |
| CCND2 | EGFR | FUBP1 | KIT | NIPBL | PTPRB | SOX2 | ZNF217 |
| CCND3 | EGRI | FUS | KLF4 | NKX2-1 | PTPRD | SPEN | ZNF703 |
| CCNE1 | EIF1AX | FYN | KLHL6 | NOTCH1 | PTPRK | SPOP | ZNFHX4 |
| CD79A | ELF3 | GAB2 | KMT2A | NOTCH3 | PTPRT | SPRED1 | ZRSR2 |
| CD79B | EMSY | GATA1 | KMT2B | NPM1 | RAC1 | SPRY1 | |

i. Stand Preparation for Tree-Dimensional Organotypic Culture

Stands for three-dimensional organotypic culture were prepared according to Example 5. A schematic representation of the stand design, including the 3D view, top view, side view, and front view, is provided in FIG. 10 with the ratio of ledge surface and cavity area of the stand from a top view shown in FIG. 11. Stands were printed with autoclavable ABS plastic using an “Objet Alaris Desktop 3D Printer.” Stands were sterilized using a standard autoclave prior their use for tissue culture.

ii. Tree-Dimensional Organotypic Culture

Three-dimensional organotypic culture was performed as described in Example 6. Briefly, dermis was obtained from discarded surgical samples and cadaver skin, and sections were frozen at −80° C. Dermis was thawed and pre-sterilized/decellularized in PBS (Life-Technologies, #AM9624) containing 1% Penicillin/Streptomycin (Fisher-Scientific, #MT30002CI) and 1% antibiotic-antimycotic (Life-Technologies, #15240062). Dermis was sterilized using gamma-radiation or 0.1% Peracetic Acid treatment for 3 hours (Sigma-Aldrich, #269336).

Prior to fibroblast re-impregnation, dermis was sectioned with a scalpel in 8 by 8 mm pieces, which were air-dried at room temperature for 3 hours (epidermal side facing down). Twenty-five thousand (25,000) human primary fibroblasts in 1 mL of fibroblast media were reintroduced into each of the dermis pieces by centrifugation (1 hour, 200 g). Cell counts were performed with a Bio-Rad TC20 cell counter (#1450102). Re-impregnated dermis was incubated for 7 days at 37° C., 5% CO2.

Dermis was flipped and mounted onto 3D-printed stands using sterilized tweezers. A thin layer of Matrigel (25-50 μL; Corning, #354235) was applied to the bottom of each of the stands. Two hundred thousand (200,000) keratinocytes in 50 μL of keratinocyte media were seeded on top of the basal membrane of the dermis. Approximately, 500 μL of organotypic culture media was added to the wells in order to create an air-liquid interface (FIG. 12). Organotypic culture media is composed of DMEM/HAM F12 (3:1; Life Technologies, #12491023; VWR, #12-615F) supplemented with 10% FBS (Fisher Scientific, #MT35010CV), 1% Penicillin/Streptomycin (Fisher-Scientific, #MT30002CI), 1% antibiotic-antimycotic (Life Technologies, #15240062), 3 μg/mL of adenine (Sigma, #A9795), 3 ng/mL of cholera toxin (EMD Millipore, #227036), 40 ng/mL of hydrocortisone (EMD Millipore, #386698), 5 μg/mL of insulin (Sigma, #I1882), 10 ng/mL of EGF (Life Technologies, #PHG0315), 5 μg/mL of Transferrin (Sigma, #T0665), 1.5 ng/mL of triiodo-L-thyronine (Sigma, #T2877) and 10 ng/mL of ciprofloxacin hydrochloride (Sigma, #PHR1044). Organotypic cultures were incubated for 7 days at 37° C., 5% CO2.

Fixation was achieved by immersing the whole culture (including stand) in 4% PFA (Electron Microscopy Sciences, #15710) overnight. Organotypic cultures was embedded in OCT (Fisher-Scientific, #4585) following incubations in 15%, 30% and 50% sucrose solutions (Fisher Scientific, #50712768). In order to analyze the structure of the organotypic cultures, blocks were sectioned vertically and 5 μm frozen sections were stained using standard H&E or immunofluorescence protocols.

iii. Immunohistochemistry (IHC) Staining

IHC was performed following standard protocol. Briefly, slides were incubated in Hematoxylin solution (ThermoFisher Scientific, #22220101) for 5 minutes. Following water washes, slides were incubated in Eosin (ThermoFisher Scientific, #22220104) solution for 1 minute. After a 3 minute wash in water, slides were dehydrated successively in 70%, 90% and 100% EtOH for 30 seconds. Slides were mounted with Cytoseal (ThermoFisher Scientific, #22050262) and allowed to dry for 24 hours. Tissue sections were submitted for pathology review.

Histology IHC stains were performed on an Automated Leica BOND III slide stainer. The stainer applies antigen retrieval (30 minutes) and antibody reagents according to validated protocols. Antibodies used in this study include: P40 (undiluted, Biocare, #BC28), KI67 (1:50 dilution, Dako, #MIB1).

iv. Extreme Limiting Dilution Assay

Extreme limiting dilution analysis (ELDA) was performed as previously described Briefly, primary keratinocytes harboring different combinations of mutations were collected from 10 cm dishes and diluted into single cell suspension and plated at concentrations of 1,000 to 1 cells per 100 μl of keratinocyte media in 96-well ultra-low attachment plates (Costar Corning, #3474). Cells were incubated at 37° C. in a 5% CO₂ for 10 days. At the end of 10 days, the number of wells showing spheres were counted by light microscope. ELDA web tool (bioinf.wehi.edu.au/software/elda) was used to determine frequencies of sphere forming cells and the statistical significance.

v. RNA Sequencing and Analysis

RNA was isolated using the RNAeasy Plus Kit (Qiagen, #74136). Sequencing libraries were prepared with QuantSeq 3^{mut} mRNA-Seq Library Prep Kit (Lexogen, #015.24). Sequencing was performed using a HiSeq 4000 sequencer (SE, 50 bp) and results were processed following standard ENCODE guidelines using Bluebee's online platform. Differential gene expression analysis and statistical analysis was performed using the DESeq2 package (39). GO terms and pathways analyses were performed with Panther Classification System (40, 41). Primary RNAseq data will be available upon publication in GEO.

vi. In vivo experiments

Injections of genome-engineered keratinocytes were performed subcutaneously in the flank of NOD-scid IL2Rgammaₘᵤₗₗ mice. Matrigel was added to 10⁶ cells in keratinocyte media (1:1 ratio) for each injection. Mice were monitored for tumor growth and tumors were extracted for subsequent analyses. This protocol is compliant with all ethical regulations and was approved by UCSF-Institutional Animal Care and Use Committee (IACUC).

2. Results

i. Individual Mutations are not Sufficient for Oncogenic Transformation

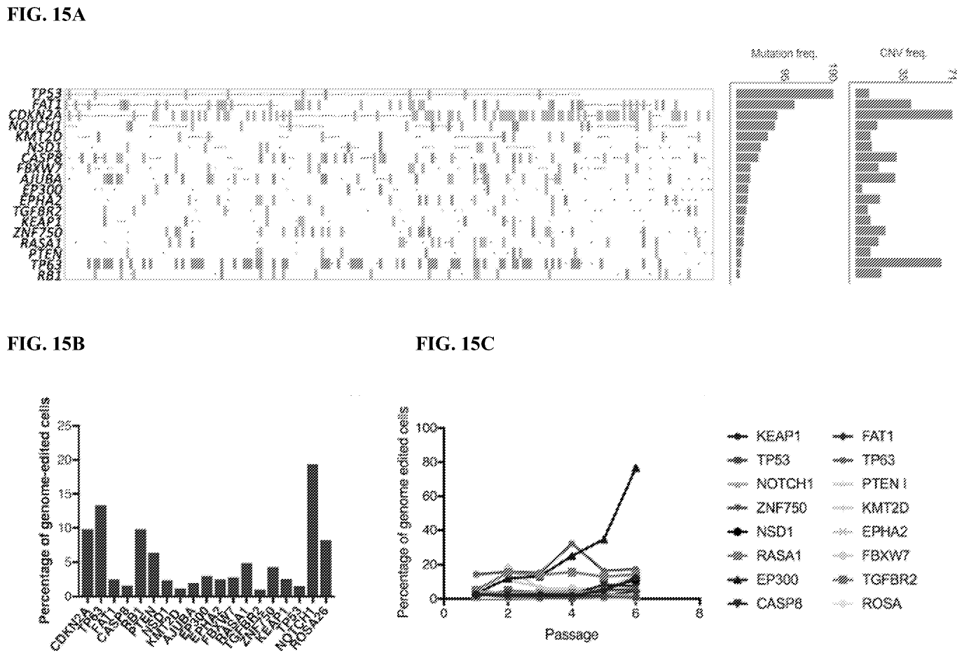

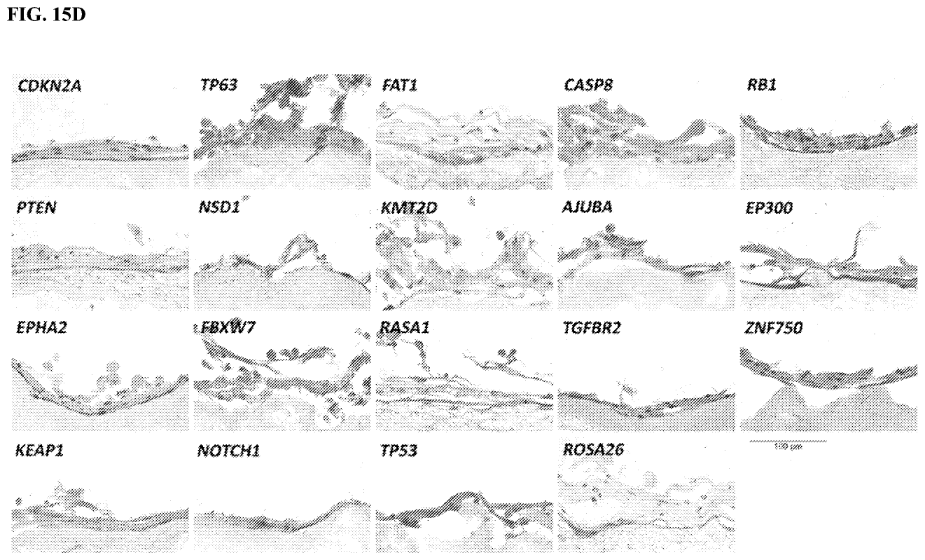

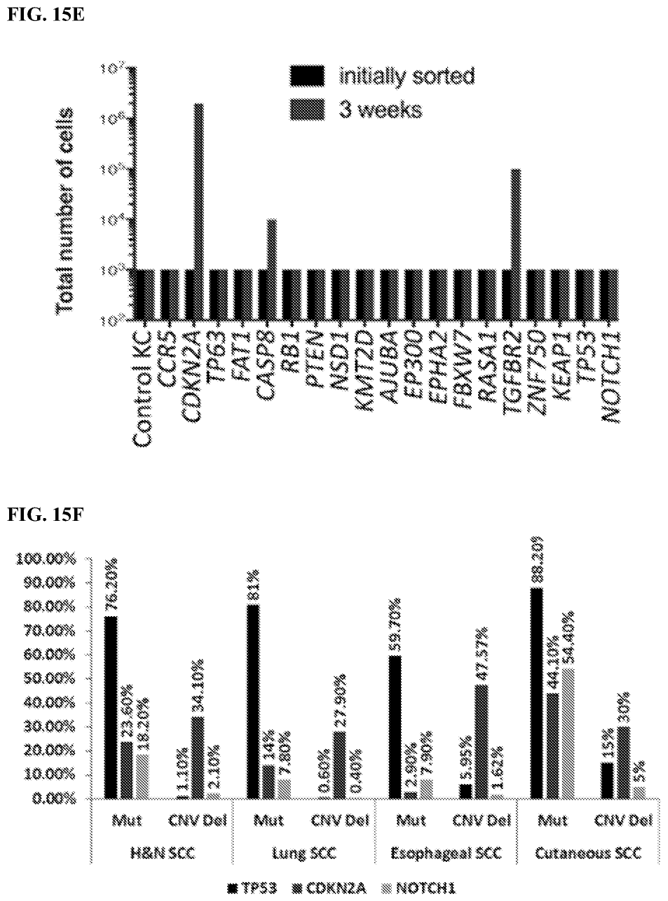

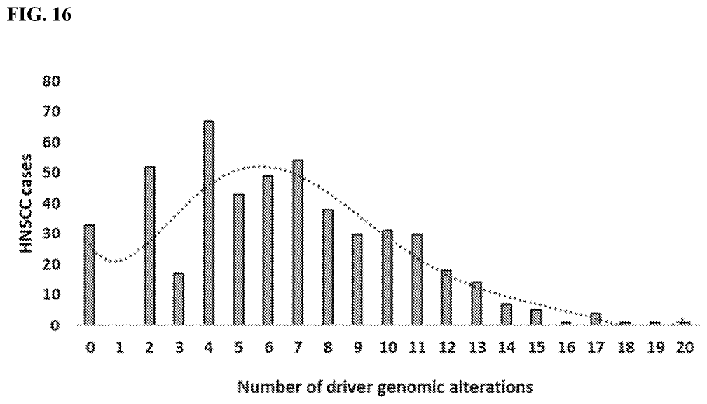

Multiple putative driver mutations in HNSCC have been identified by algorithms that predict the significance of recurrent mutations in large sets of sequenced tumors, the majority of which function as tumor suppressors (14, 15) (FIG. 15A). To evaluate the biological effect of 18 of the most common and significantly mutated putative driver mutations, we utilized the CRISPR/Cas9 system for targeted gene disruption of each of these genes individually in human primary foreskin keratinocytes. A mutation in at least one of these 18 genes is present in ˜93% of HNSCC cases (TCGA data), and ˜83% of these tumors show alterations in at least three of these genes (FIG. 16). The editing efficiency in primary keratinocytes for any individual gene ranged from 1-20% (19) (FIG. 15B). When grown in non-sorted populations, the majority of these single mutations did not provide a selective advantage over unedited cells in the same culture; only EP300^{mut} cells overtook non-edited keratinocytes with repeat passaging (FIG. 15C). To assess whether the disruption of any of these single genes is sufficient to trigger other characteristics of malignant transformation, particularly invasion, organotypic cultures with non-sorted, genome-edited primary keratinocytes were generated. In this system, primary keratinocytes are grown on the apical basement membrane of fibroblast-impregnated human dermis and form a stratified squamous epithelium, thus recapitulating the three dimensional structure of human skin or squamous mucosa (20). Among cultures of non-sorted, genome-edited keratinocytes, no single gene mutation, including EP300, was sufficient to induce lesions with characteristics of preneoplasia at the histological level as assessed by a blinded pathologist (FIG. 15D). FACS selection was then used to facilitate isolation of >90% pure cultures of edited keratinocytes (FIG. 15B). To assess whether single gene disruption was sufficient to immortalize cells, we performed serial passaging of FACS-purified, edited keratinocytes (FIG. 15E). In this case, only CDKN2Aₘᵤₜ, TGFBR2ₘᵤₜ, and CASP8^{mut} keratinocytes passaged beyond control, unedited or CCR5^{mut} genome-edited keratinocytes. After a few passages, TGFBR2^{mut} and CASP8^{mut} keratinocytes showed limited proliferation, while CDKN2A^{mut} cells continued to actively grow. Taken together, these data demonstrate that individual driver mutations are insufficient for the histopathological features of pre-cancerous or cancerous lesions, consistent with the previous finding of driver mutations in normal appearing stratified squamous epithelium (17, 18). Thus, it is hypothesized that combinations of mutations are likely required to induce the histopathological changes typical of pre-cancerous or cancerous lesions.

ii. Combinations of Mutations Transform Keratinocytes

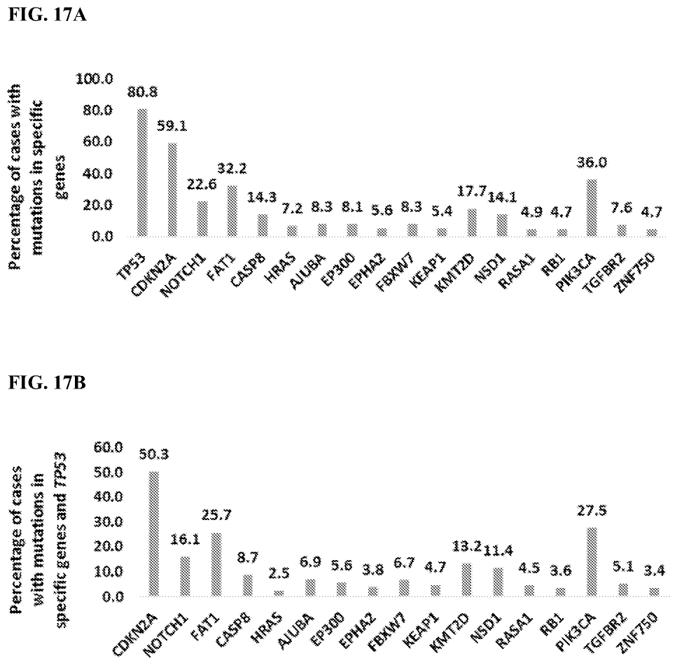

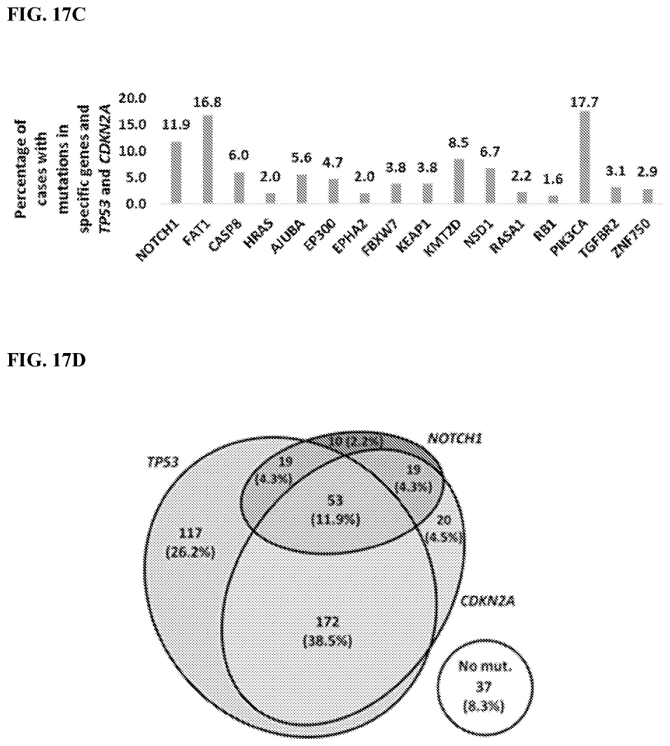

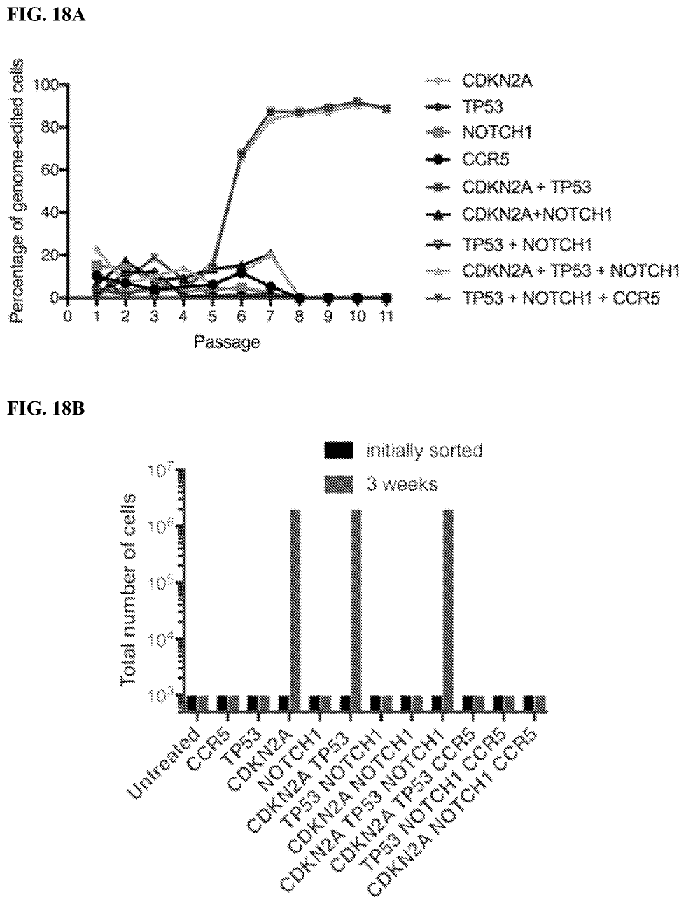

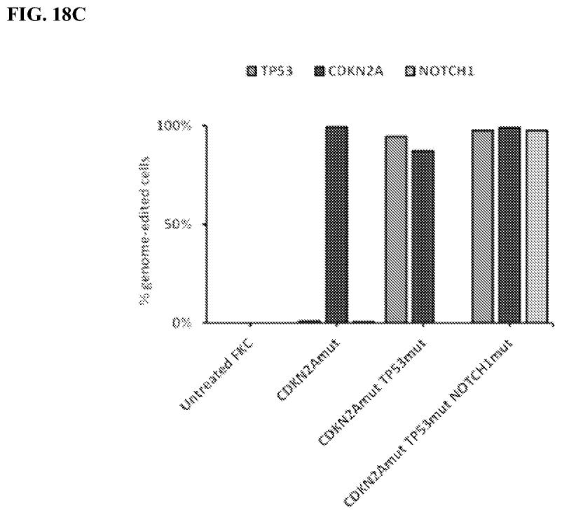

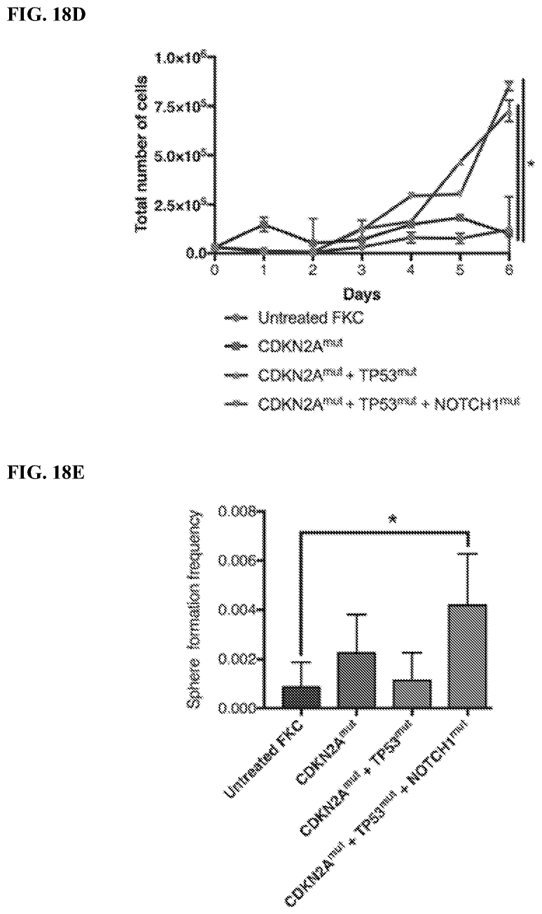

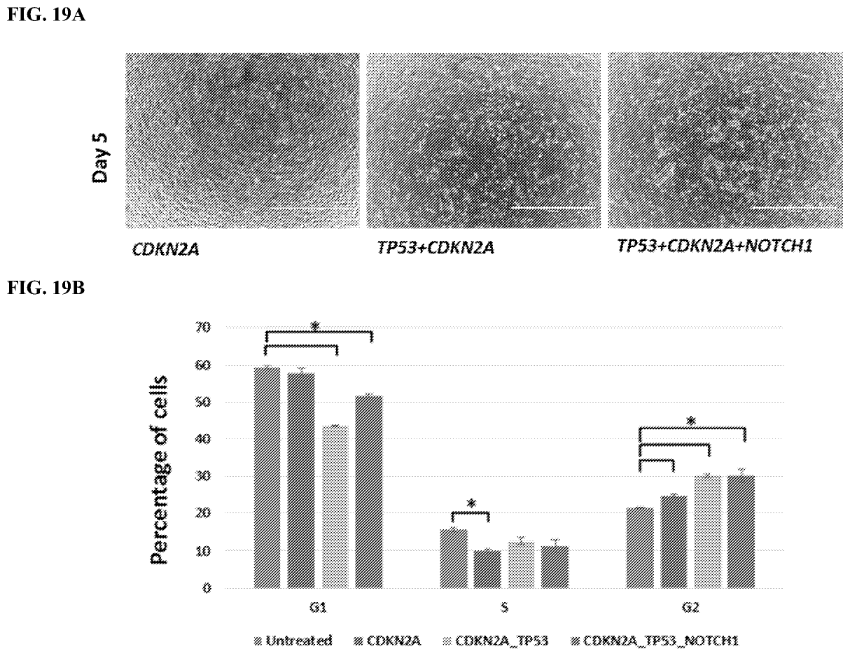

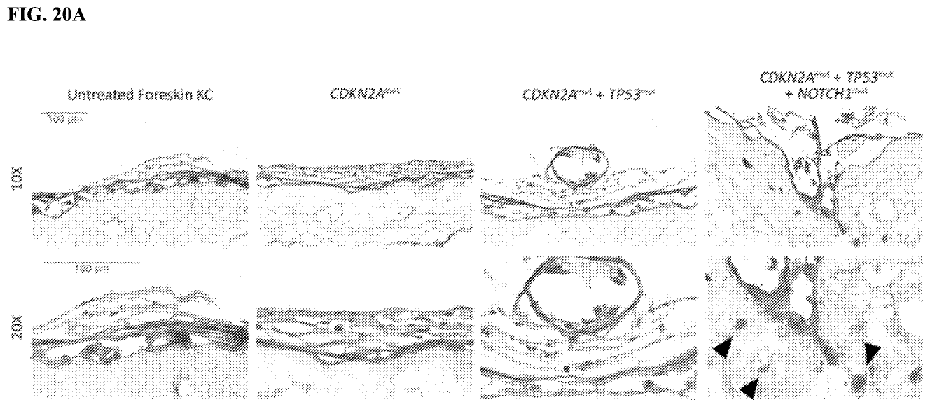

TCGA data were next interrogated to identify the most common combinations of driver mutations in HPV-negative HNSCC. 90% of the cases with genomic alterations in CDKN2A also harbor mutations in TP53, suggesting that these two events may cooperate in SCC formation. Among tumors with TP53 and CDKN2A mutations, mutations in NOTCH1 are among the most common co-occurring genomic events in HNSCC and other non-viral related SCC (8, 12, 21, 22) (FIG. 15F, FIG. 17A-17D). To determine whether specific combinations of these mutations are sufficient to induce preneoplasia or cancer, mutations in CDKN2A, TP53, and NOTCH1 were introduced, both individually and in combination, in primary human keratinocytes. Keratinocytes harboring mutations in CDKN2A and TP53 (CT^{mut} cells) or CDKN2A, TP53, and NOTCH1 (CTN^{mut} cells) gained a selective advantage in vitro and were immortalized in two-dimensional culture, while keratinocytes with other combinations of these three genetic alterations ceased proliferating within a few passages (FIG. 18A). To further assess the biological effects of these mutations, populations of genome-engineered keratinocytes harboring combinations of mutations were FACS-sorted. Only CDKN2A^{mut}, CT^{mut}, and CTN^{mut} keratinocytes could be recovered and sequentially passaged (FIG. 18B). Sanger sequencing and TIDE analysis confirmed the expected genomic alterations in all sorted and expanded conditions (FIG. 18C). These results indicate that TP53^{mut} or TP53/NOTCH1^{mut} keratinocytes required the additional mutation of CDKN2A for survival. In a time course performed in two dimensional culture, at early time points there were no differences in cell numbers between any of the mutant conditions and control edited keratinocytes (FIG. 18D), although some differences were detected in cell-cycle distribution (FIG. 19). However, while normal and CDKN2A^{mut} keratinocytes exhibited contact inhibition, CT^{mut} and CTN^{mut} keratinocytes lost contact inhibition and continued to proliferate at later time points (FIG. 18D, FIG. 19). In order to identify alterations in the clonogenic potential of edited keratinocytes, sphere-forming assays using low-attachment plates were performed. CTN^{mut} keratinocytes showed a higher sphere-forming rate than control edited, CDKN2A^{mut}, and CT^{mut} keratinocytes (FIG. 18E). These results suggest that distinct combinations of mutations permit the acquisition of a selective advantage in distinct environmental contexts.

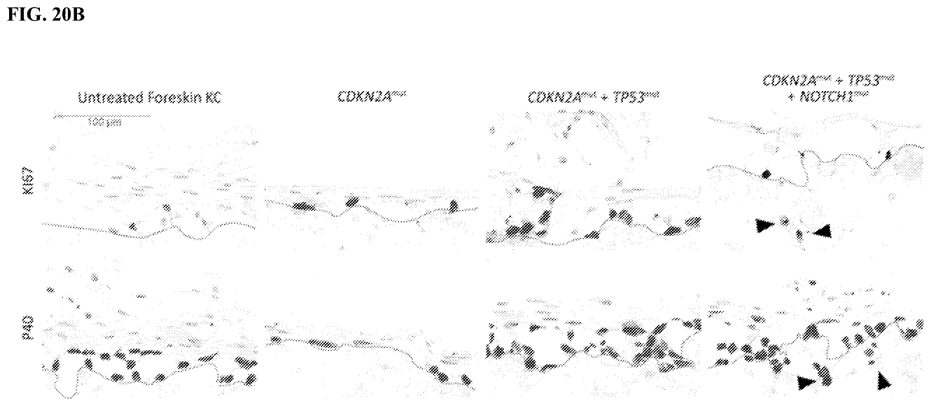

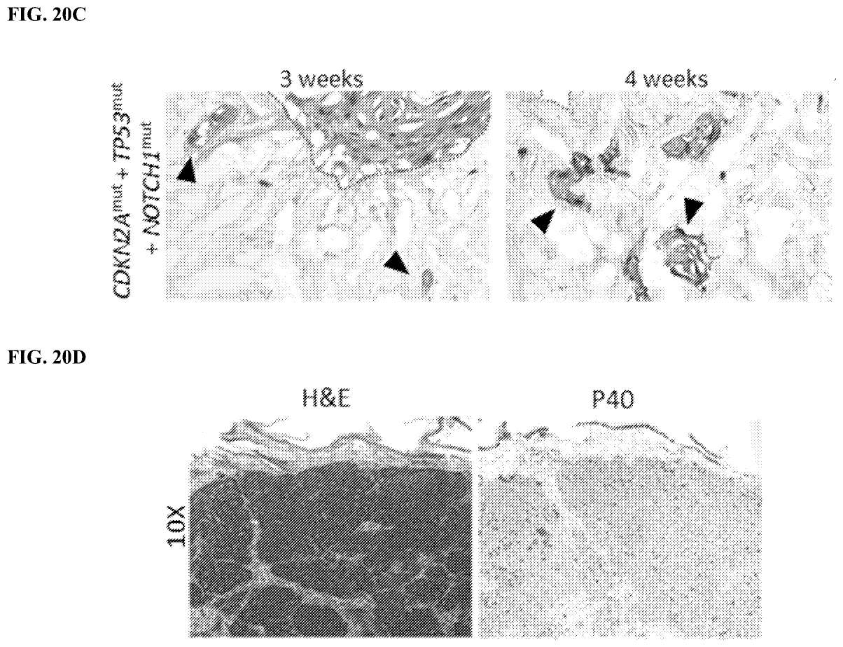

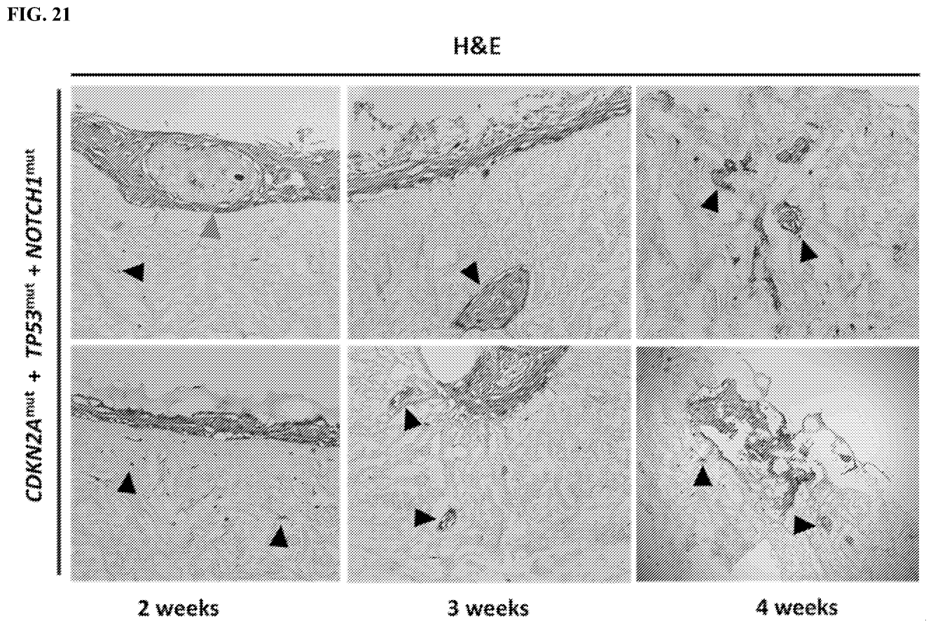

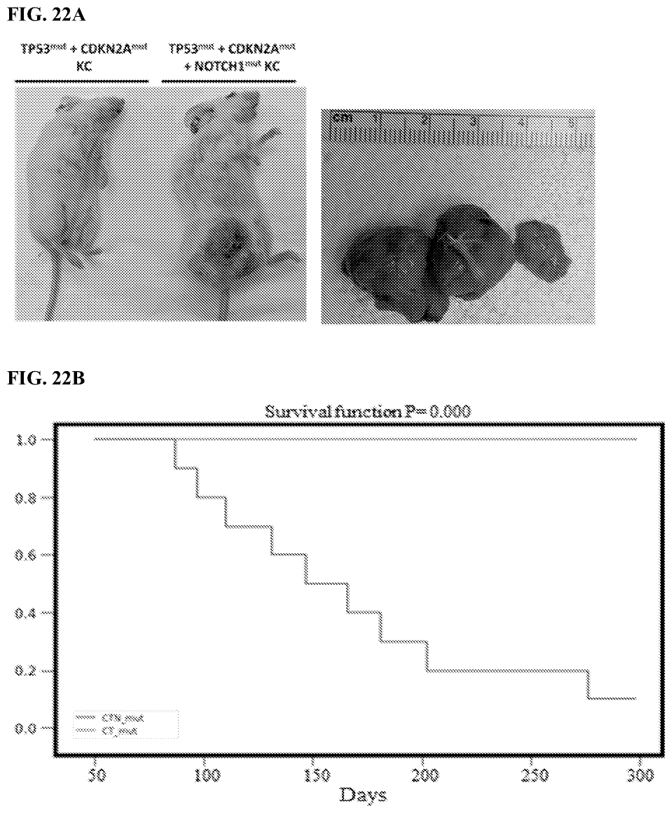

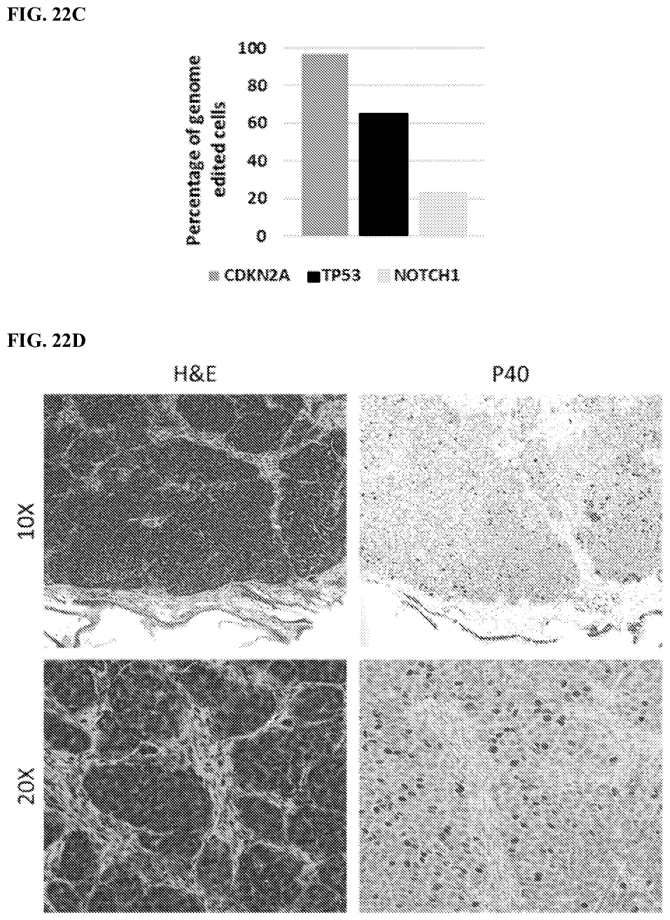

The defining histopathological hallmark of SCC is invasion of tumor cells through the basement membrane into the underlying mesenchymal tissue. To assess whether different combinations of CDKN2A, TP53, and NOTCH1 mutations are sufficient to induce invasion, genome-edited keratinocytes were grown as epithelial layers in organotypic cultures. Dermal invasion of individual cells or small groups of cells was only detected in cultures of CTN^{mut} keratinocytes within a week of culture, confirming that the combination of these three mutations is sufficient to induce a cancer-like phenotype in human keratinocytes (FIG. 20A). CT^{mut} keratinocytes were able to form pre-neoplastic intra-epithelial lesions with enhanced proliferation, keratin pearls, and enlarged nuclei, but did not invade the basement membrane (FIG. 20A). To characterize the impact of our engineered mutations on proliferation and differentiation state in organotypic cultures, we stained for Ki67 and CD40 (ΔNp63), a marker of basal keratinocytes (FIG. 20B). CTN^{mut} keratinocytes that have invaded the dermis express both of these markers; demonstrating that invasive cells are proliferating and retain expression of basal squamous markers. After 3 weeks of continued in vitro culture CTN^{mut} keratinocytes form larger nests of invasive cells (FIG. 20C, FIG. 21). Further, CTN^{mut} keratinocytes injected into Nod-Scid-gamma mice were capable of tumor formation in vivo, whereas CT^{mut} cells were not. Mice injected with CTN^{mut} keratinocytes showed a poor survival rate (FIG. 20D, FIG. 22). Sanger sequencing confirmed the presence of the predicted mutations in CDKN2A, TP53, and NOTCH1 in tumor cells grown in vivo (FIG. 22C).

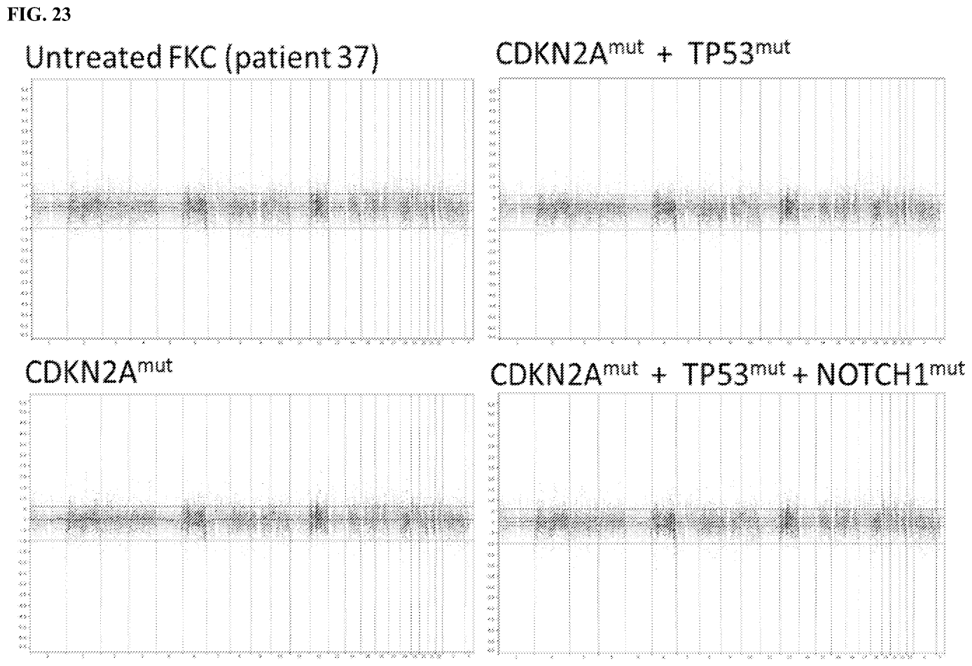

It is possible that, in the course of genome editing, additional oncogenic mutations accrue and contribute to the phenotypes we observed. In order to rule out the contribution of continued evolution and acquisition of additional genomic alterations in our edited and sorted keratinocytes, we performed capture-based next generation sequencing targeting a panel of 479 cancer-related genes and regulatory elements at a depth of 500λ. We detected no mutations or copy number alterations other than those intentionally introduced with CRISPR/Cas9 editing (FIG. 23, Tables 6-7). In order to ensure that the transformation of keratinocytes was due to disruption of the targeted genes and not non-specific genomic instability at loci not captured in the targeted panel sequencing, we created edited keratinocytes with small guide RNAs that target the same three genes but at distinct loci and we obtained similar results as above (data not shown). In addition, this oncogenic phenotype was observed as early as 20 days after editing without repeated passages, the earliest point we had sufficient numbers of cells to assay, thus reducing the likelihood of selection of a small number of clones with additional mutations.

TABLE 8
UCSF500 Panel Sequencing Results.
CDKN2A^{mut}

| CHROM | REF | ALT | Func | Variant | TranscriptID | ExonicFunc | AAChange |
|---|---|---|---|---|---|---|---|
| chr9 | CG | C | exonic | CDKN2A | NM_000077 | frameshift | CDKN2A:NM_000077:exon1:c.136delC:p.R46fs |
| | | | | p.R46fs | | deletion | CDKN2A:NM_001195132:exon1:c.136delC:p.R46fs |
| | | | | | | | CDKN2A:NM_058197:exon1:c.136delC:p.R46fs |

CDKN2A^{mut} + TP53^{mut}

| CHROM | REF | ALT | Func | Variant | TranscriptID | ExonicFunc | AAChange |
|---|---|---|---|---|---|---|---|
| chr17 | AGGC | A | exonic | TP53 | NM_000546 | nonframeshift | TP53:NM_001126115:exon2:c.270_272del:p.90_91del |
| | | | | p.222_223del | | deletion | TP53:NM_001126116:exon2:c.270_272del:p.90_91del |
| | | | | | | | TP53:NM_001126117:exon2:c.270_272del:p.90_91del |
| | | | | | | | TP53:NM_001276697:exon2:c.189_191del:p.63_64del |
| | | | | | | | TP53:NM_001276698:exon2:c.189_191del:p.63_64del |
| | | | | | | | TP53:NM_001276699:exon2:c.189_191del:p.63_64del |
| | | | | | | | TP53:NM_001126118:exon5:c.549_551del:p.183_184del |
| | | | | | | | TP53:NM_000546:exon6:c.666_668del:p.222_223del |
| | | | | | | | TP53:NM_001126112:exon6:c.666_668del:p.222_223del |
| | | | | | | | TP53:NM_001126113:exon6:c.666_668del:p.222_223del |
| | | | | | | | TP53:NM_001126114:exon6:c.666_668del:p.222_223del |
| | | | | | | | TP53:NM_001276695:exon6:c.549_551del:p.183_184del |
| | | | | | | | TP53:NM_001276696:exon6:c.549_551del:p.183_184del |
| | | | | | | | TP53:NM_001276760:exon6:c.549_551del:p.183_184del |
| | | | | | | | TP53:NM_001276761:exon6:c.549_551del:p.183_184del |
| chr17 | CCTCAGGC | C | exonic | TP53 | NM_000546 | nonframeshift | TP53:NM_001126115:exon2:c.268_276del:p.90_92del |
| | CC | | | p.222_224del | | deletion | TP53:NM_001126116:exon2:c.268_276del:p.90_92del |
| | | | | | | | TP53:NM_001126117:exon2:c.268_276del:p.90_92del |
| | | | | | | | TP53:NM_001276697:exon2:c.187_195del:p.63_65del |
| | | | | | | | TP53:NM_001276698:exon2:c.187_195del:p.63_65del |
| | | | | | | | TP53:NM_001276699:exon2:c.187_195del:p.63_65del |
| | | | | | | | TP53:NM_001126118:exon5:c.547_555del:p.183_185del |
| | | | | | | | TP53:NM_000546:exon6:c.664_672del:p.222_224del |
| | | | | | | | TP53:NM_001126112:exon6:c.664_672del:p.222_224del |
| | | | | | | | TP53:NM_001126113:exon6:c.664_672del:p.222_224del |
| | | | | | | | TP53:NM_001126114:exon6:c.664_672del:p.222_224del |
| | | | | | | | TP53:NM_001276695:exon6:c.547_555del:p.183_185del |
| | | | | | | | TP53:NM_001276696:exon6:c.547_555del:p.183_185del |
| | | | | | | | TP53:NM_001276760:exon6:c.547_555del:p.183_185del |
| | | | | | | | TP53:NM_001276761:exon6:c.547_555del:p.183_185del |
| chr17 | GGCGGC | G | exonic | TP53 | NM_000546 | frameshift | TP53:NM_001126115:exon2:c.267_271del:p.E89fs |
| | | | | p.E221fs | | deletion | TP53:NM_001126116:exon2:c.267_271del:p.E89fs |
| | | | | | | | TP53:NM_001126117:exon2:c.267_271del:p.E89fs |
| | | | | | | | TP53:NM_001276697:exon2:c.186_190del:p.E62fs |
| | | | | | | | TP53:NM_001276698:exon2:c.186_190del:p.E62fs |
| | | | | | | | TP53:NM_001276699:exon2:c.186_190del:p.E62fs |
| | | | | | | | TP53:NM_001126118:exon5:c.546_550del:p.E182fs |
| | | | | | | | TP53:NM_000546:exon6:c.663_667del:p.E221fs |
| | | | | | | | TP53:NM_001126112:exon6:c.663_667del:p.E221fs |
| | | | | | | | TP53:NM_001126113:exon6:c.663_667del:p.E221fs |
| | | | | | | | TP53:NM_001126114:exon6:c.663_667del:p.E221fs |
| | | | | | | | TP53:NM_001276695:exon6:c.546_550del:p.E182fs |
| | | | | | | | TP53:NM_001276696:exon6:c.546_550del:p.E182fs |
| | | | | | | | TP53:NM_001276760:exon6:c.546_550del:p.E182fs |
| | | | | | | | TP53:NM_001276761:exon6:c.546_550del:p.E182fs |
| chr17 | TGCAAACC | T | exonic | TP53 | NM_000546 | frameshift | TP53:NM_001126115:exon2:c.260_276del:p.P87fs |
| | AGACCTCA | | | p.P219fs | | deletion | TP53:NM_001126116:exon2:c.260_276del:p.P87fs |
| | GGCGGCTC | | | | | | TP53:NM_001126117:exon2:c.260_276del:p.P87fs |
| | ATAGG | | | | | | TP53:NM_001276697:exon2:c.179_195del:p.P60fs |
| | | | | | | | TP53:NM_001276698:exon2:c.179_195del:p.P60fs |
| | | | | | | | TP53:NM_001276699:exon2:c.179_195del:p.P60fs |
| | | | | | | | TP53:NM_001126118:exon5:c.539_555del:p.P180fs |
| | | | | | | | TP53:NM_000546:exon6:c.656_672del:p.P219fs |
| | | | | | | | TP53:NM_001126112:exon6:c.656_672del:p.P219fs |
| | | | | | | | TP53:NM_001126113:exon6:c.656_672del:p.P219fs |
| | | | | | | | TP53:NM_001126114:exon6:c.656_672del:p.P219fs |
| | | | | | | | TP53:NM_001276695:exon6:c.539_555del:p.P180fs |
| | | | | | | | TP53:NM_001276696:exon6:c.539_555del:p.P180fs |
| | | | | | | | TP53:NM_001276760:exon6:c.539_555del:p.P180fs |
| | | | | | | | TP53:NM_001276761:exon6:c.539_555del:p.P180fs |
| chr17 | AG | A | exonic | TP53 | NM_000546 | frameshift | TP53:NM_001126115:exon2:c.272delC:p.P91fs |
| | | | | p.P223fs | | deletion | TP53:NM_001126116:exon2:c.272delC:p.P91fs |
| | | | | | | | TP53:NM_001126117:exon2:c.272delC:p.P91fs |
| | | | | | | | TP53:NM_001276697:exon2:c.191delC:p.P64fs |
| | | | | | | | TP53:NM_001276698:exon2:c.191delC:p.P64fs |
| | | | | | | | TP53:NM_001276699:exon2:c.191delC:p.P64fs |
| | | | | | | | TP53:NM_001126118:exon5:c.551delC:p.P184fs |
| | | | | | | | TP53:NM_000546:exon6:c.668delC:p.P223fs |
| | | | | | | | TP53:NM_001126112:exon6:c.668delC:p.P223fs |
| | | | | | | | TP53:NM_001126113:exon6:c.668delC:p.P223fs |
| | | | | | | | TP53:NM_001126114:exon6:c.668delC:p.P223fs |
| | | | | | | | TP53:NM_001276695:exon6:c.551delC:p.P184fs |
| | | | | | | | TP53:NM_001276696:exon6:c.551delC:p.P184fs |
| | | | | | | | TP53:NM_001276760:exon6:c.551delC:p.P184fs |
| | | | | | | | TP53:NM_001276761:exon6:c.551delC:p.P184fs |
| chr17 | G | GC | exonic | TP53 | NM_000546 | frameshift | TP53:NM_001126115:exon2:c.271_272insG:p.P91fs |
| | | | | p.P223fs | | deletion | TP53:NM_001126116:exon2:c.271_272insG:p.P91fs |
| | | | | | | | TP53:NM_001126117:exon2:c.271_272insG:p.P91fs |
| | | | | | | | TP53:NM_001276697:exon2:c.190_191insG:p.P64fs |
| | | | | | | | TP53:NM_001276698:exon2:c.190_191insG:p.P64fs |
| | | | | | | | TP53:NM_001276699:exon2:c.190_191insG:p.P64fs |
| | | | | | | | TP53:NM_001126118:exon5:c.550_551insG:p.P184fs |
| | | | | | | | TP53:NM_000546:exon6:c.667_668insG:p.P223fs |
| | | | | | | | TP53:NM_001126112:exon6:c.667_668insG:p.P223fs |
| | | | | | | | TP53:NM_001126113:exon6:c.667_668insG:p.P223fs |
| | | | | | | | TP53:NM_001126114:exon6:c.667_668insG:p.P223fs |
| | | | | | | | TP53:NM_001276695:exon6:c.550_551insG:p.P184fs |
| | | | | | | | TP53:NM_001276696:exon6:c.550_551insG:p.P184fs |
| | | | | | | | TP53:NM_001276760:exon6:c.550_551insG:p.P184fs |
| | | | | | | | TP53:NM_001276761:exon6:c.550_551insG:p.P184fs |
| chr17 | CAG | C | exonic | TP53 | NM_000546 | frameshift | TP53:NM_001126115:exon2:c.272_273del:p.P91fs |
| | | | | p.P223fs | | deletion | TP53:NM_001126116:exon2:c.272_273del:p.P91fs |
| | | | | | | | TP53:NM_001126117:exon2:c.272_273del:p.P91fs |
| | | | | | | | TP53:NM_001276697:exon2:c.191_192del:p.P64fs |
| | | | | | | | TP53:NM_001276698:exon2:c.191_192del:p.P64fs |
| | | | | | | | TP53:NM_001276699:exon2:c.191_192del:p.P64fs |
| | | | | | | | TP53:NM_001126118:exon5:c.551_552del:p.P184fs |
| | | | | | | | TP53:NM_000546:exon6:c.668_669del:p.P223fs |
| | | | | | | | TP53:NM_001126112:exon6:c.668_669del:p.P223fs |
| | | | | | | | TP53:NM_001126113:exon6:c.668_669del:p.P223fs |
| | | | | | | | TP53:NM_001126114:exon6:c.668_669del:p.P223fs |
| | | | | | | | TP53:NM_001276695:exon6:c.551_552del:p.P184fs |
| | | | | | | | TP53:NM_001276696:exon6:c.551_552del:p.P184fs |
| | | | | | | | TP53:NM_001276760:exon6:c.551_552del:p.P184fs |
| | | | | | | | TP53:NM_001276761:exon6:c.551_552del:p.P184fs |
| chr9 | GACCGTA | G | exonic | CDKN2A | NM_000077 | nonframeshift | CDKN2A:NM_000077:exon1:c.130_135del:p.44_45del |
| | | | | p.44_45del | | deletion | CDKN2A:NM_001195132:exon1:c.130_135del:p.44_45del |
| | | | | | | | CDKN2A:NM_058197:exon1:c.130_135del:p.44_45del |
| chr9 | TCCGA | T | exonic | CDKN2A | NM_000077 | frameshift | CDKN2A:NM_000077:exon1:c.135_138del:p.G45fs |
| | | | | p.G45fs | | deletion | CDKN2A:NM_001195132:exon1:c.135_138del:p.G45fs |
| | | | | | | | CDKN2A:NM_058197:exon1:c.135_138del:p.G45fs |
| chr9 | A | AT | exonic | CDKN2A | NM_000077 | frameshift | CDKN2A:NM_000077:exon1:c.134_135insA:p.G45fs |
| | | | | p.G45fs | | deletion | CDKN2A:NM_001195132:exon1:c.134_135insA:p.G45fs |
| | | | | | | | CDKN2A:NM_058197:exon1:c.134_135insA:p.G45fs |
| chr9 | G | GA | exonic | CDKN2A | NM_000077 | frameshift | CDKN2A:NM_000077:exon1:c.135dupT:p.R46fs |
| | | | | p.R46fs | | deletion | CDKN2A:NM_001195132:exon1:c.135dupT:p.R46fs |
| | | | | | | | CDKN2A:NM_058197:exon1:c.135dupT:p.R46fs |
| chr9 | CG | C | exonic | CDKN2A | NM_000077 | frameshift | CDKN2A:NM_000077:exon1:c.136delC:p.R46fs |
| | | | | p.R46fs | | deletion | CDKN2A:NM_001195132:exon1:c.136delC:p.R46fs |
| | | | | | | | CDKN2A:NM_058197:exon1:c.136delC:p.R46fs |

CDKN2A^{mut} + TP53^{mut} + NOTCH1^{mut}

| CHROM | REF | ALT | Func | Variant | TranscriptID | ExonicFunc | AAChange |
|---|---|---|---|---|---|---|---|
| chr17 | GGC | G | exonic | TP53 | NM_000546 | frameshift | TP53:NM_001126115:exon2:c.270_271del:p.P90fs |
| | | | | p.P222fs | | deletion | TP53:NM_001126116:exon2:c.270_271del:p.P90fs |
| | | | | | | | TP53:NM_001126117:exon2:c.270_271del:p.P90fs |
| | | | | | | | TP53:NM_001276697:exon2:c.189_190del:p.P63fs |
| | | | | | | | TP53:NM_001276698:exon2:c.189_190del:p.P63fs |
| | | | | | | | TP53:NM_001276699:exon2:c.189_190del:p.P63fs |
| | | | | | | | TP53:NM_001126118:exon5:c.549_550del:p.P183fs |
| | | | | | | | TP53:NM_000546:exon6:c.666_667del:p.P222fs |
| | | | | | | | TP53:NM_001126112:exon6:c.666_667del:p.P222fs |
| | | | | | | | TP53:NM_001126113:exon6:c.666_667del:p.P222fs |
| | | | | | | | TP53:NM_001126114:exon6:c.666_667del:p.P222fs |
| | | | | | | | TP53:NM_001276695:exon6:c.549_550del:p.P183fs |
| | | | | | | | TP53:NM_001276696:exon6:c.549_550del:p.P183fs |
| | | | | | | | TP53:NM_001276760:exon6:c.549_550del:p.P183fs |
| | | | | | | | TP53:NM_001276761:exon6:c.549_550del:p.P183fs |
| chr17 | G | GT | | TP53 | NM_000546 | frameshift | TP53:NM_001126115:exon2:c.271_272insA:p.P91fs |
| | | | | p.P222fs | | deletion | TP53:NM_001126116:exon2:c.271_272insA:p.P91fs |
| | | | | | | | TP53:NM_001126117:exon2:c.271_272insA:p.P91fs |
| | | | | | | | TP53:NM_001276697:exon2:c.190_191insA:p.P64fs |
| | | | | | | | TP53:NM_001276698:exon2:c.190_191insA:p.P64fs |
| | | | | | | | TP53:NM_001276699:exon2:c.190_191insA:p.P64fs |
| | | | | | | | TP53:NM_001126118:exon5:c.550_551insA:p.P184fs |
| | | | | | | | TP53:NM_000546:exon6:c.667_668insA:p.P223fs |
| | | | | | | | TP53:NM_001126112:exon6:c.667_668insA:p.P223fs |
| | | | | | | | TP53:NM_001126113:exon6:c.667_668insA:p.P223fs |
| | | | | | | | TP53:NM_001126114:exon6:c.667_668insA:p.P223fs |
| | | | | | | | TP53:NM_001276695:exon6:c.550_551insA:p.P184fs |
| | | | | | | | TP53:NM_001276696:exon6:c.550_551insA:p.P184fs |
| | | | | | | | TP53:NM_001276760:exon6:c.550_551insA:p.P184fs |
| | | | | | | | TP53:NM_001276761:exon6:c.550_551insA:p.P184fs |
| chr9 | TCCGA | T | exonic | CDKN2A | NM_000077 | frameshift | CDKN2A:NM_000077:exon1:c.135_138del:p.G45fs |
| | | | | p.G45fs | | deletion | CDKN2A:NM_001195132:exon1:c.135_138del:p.G45fs |
| | | | | | | | CDKN2A:NM_058197:exon1:c.135_138del:p.G45fs |
| chr9 | TAC | T | exonic | NOTCH1 | NM_017617 | frameshift | NOTCH1:NM_017617:exon6:c.875_876del:p.C292fs |
| | | | | p.C292fs | | deletion | |
| chr9 | G | GT | exonic | NOTCH1 | NM_017617 | frameshift | NOTCH1:NM_017617:exon6:c.877dupA:p.T293fs |
| | | | | p.T293fs | | deletion | |

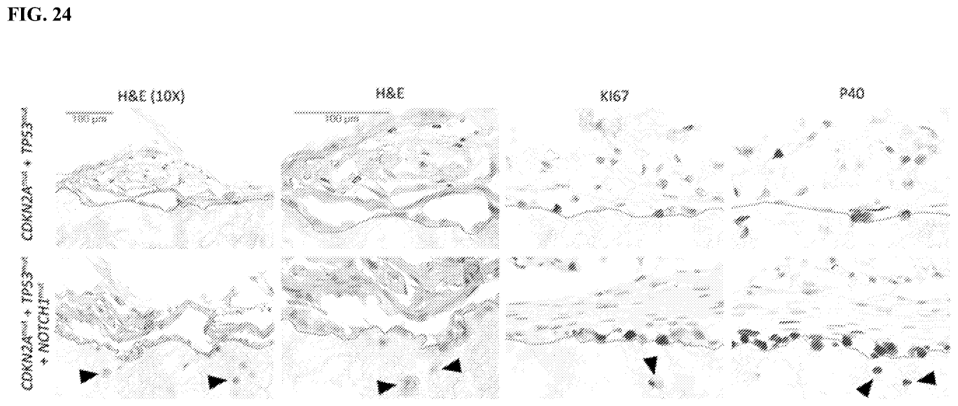

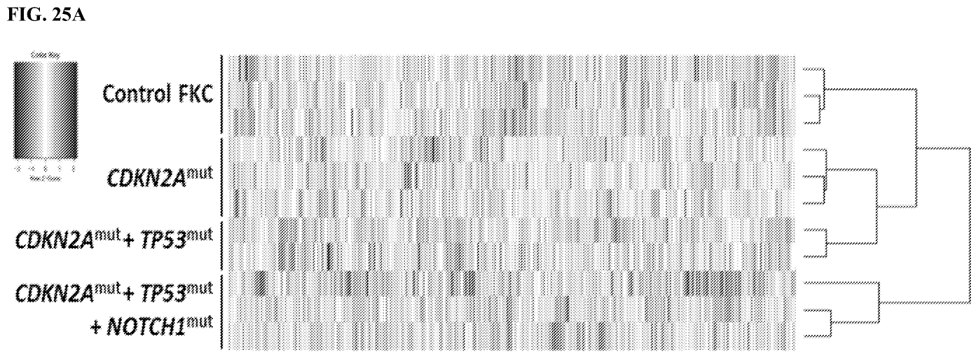

Previous studies have highlighted the similarities between SCC arising from different anatomical sites (14). To test the generalizability of our findings in another tissue of origin, we induced mutations in TP53, CDKN2A, and NOTCH1 in primary human tonsil keratinocytes and observed a similar invasive phenotype as seen in foreskin keratinocytes (FIG. 24). Based on similar mutation patterns detected in different non-viral SCC tumor types, this mutational pathway is likely operant across distinct tissues of origin. Thus, mutations in TP53 and CDKN2A are sufficient to immortalize but not transform keratinocytes, and the addition of NOTCH1 mutation is sufficient for the progression to cancer.

iii. Mechanisms of Keratinocyte Transformation

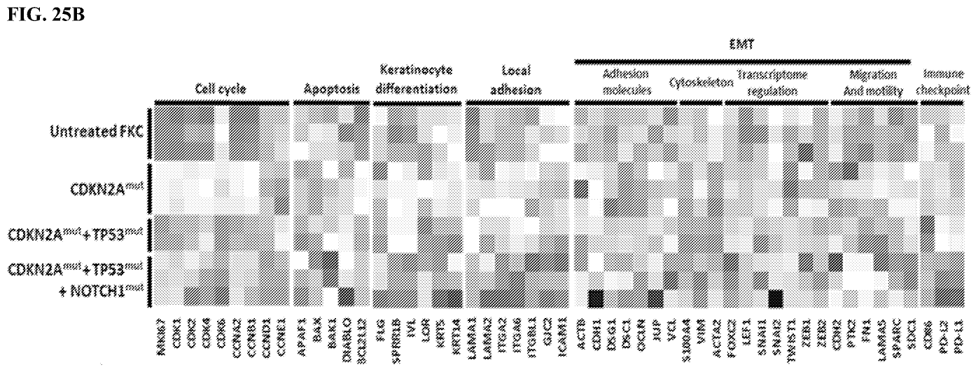

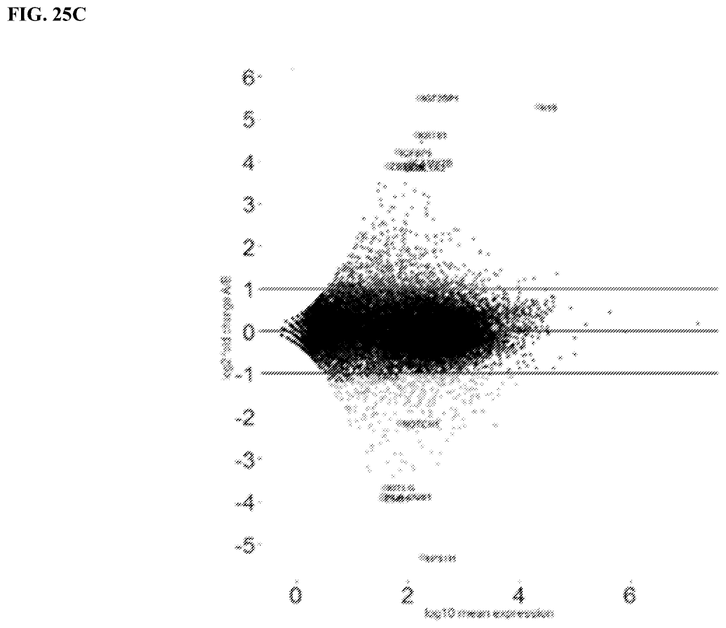

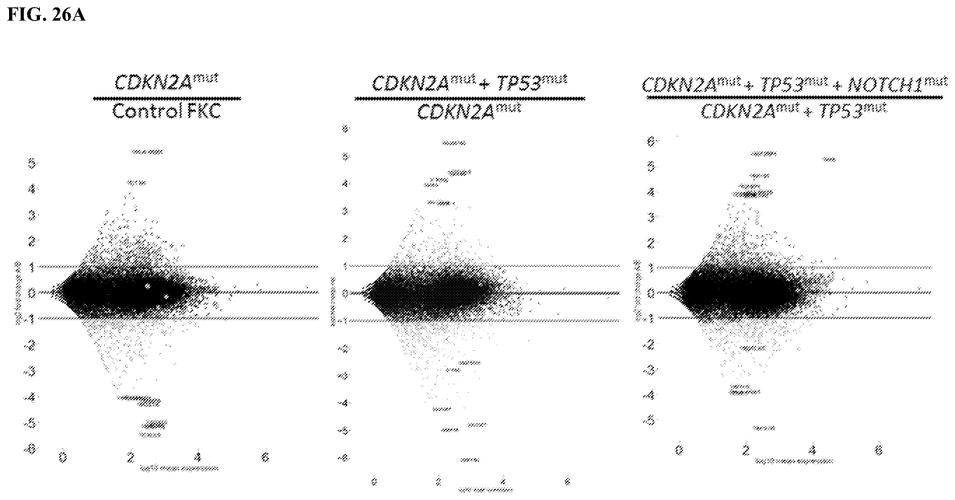

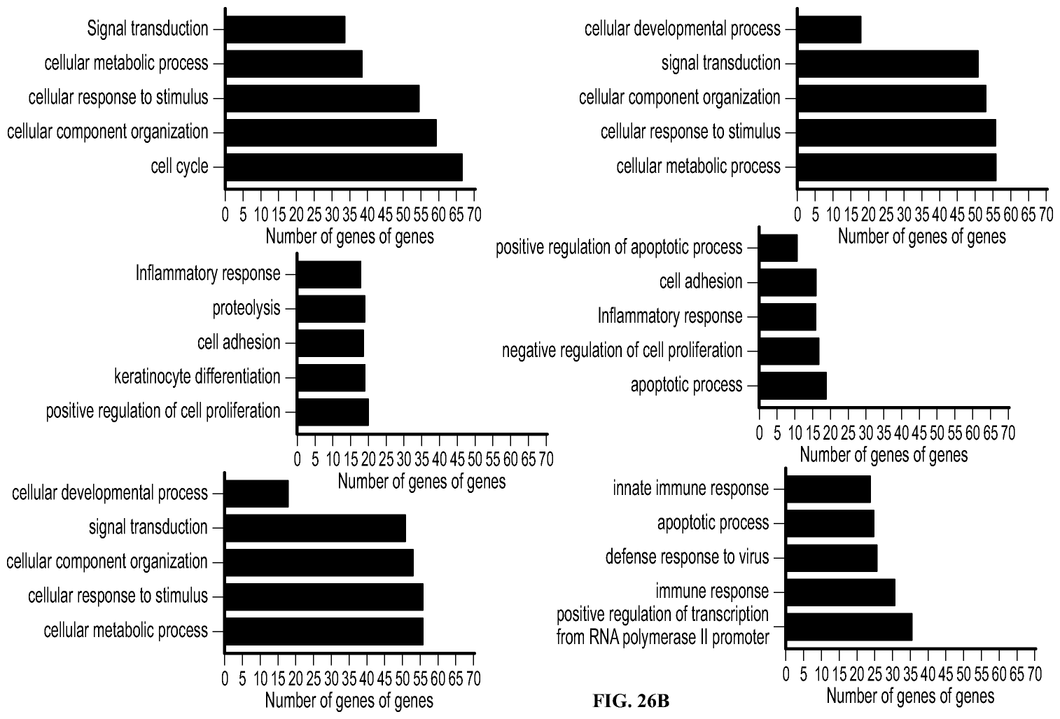

In order to understand how the transcriptome is reprogrammed in response to the acquisition of each mutation, we performed whole-genome RNA sequencing (RNA-seq) analysis on pure populations of genome-engineered primary human keratinocytes harboring combinations of mutations in TP53, CDKN2A, and NOTCH1. Unsupervised clustering demonstrated that CTN^{mut} keratinocytes are the most transcriptomically distinct in comparison to CT^{mut}, CDKN2Aₘᵤₜ, and unedited keratinocytes (FIG. 25A). Mutation in CDKN2A alone resulted in significant changes in 1,051 genes (adjusted p-value <0.05, at least twofold), including an enrichment of genes promoting cell cycle progression and a slight downregulation of genes involved in keratinocyte differentiation (FIG. 25B). The additional mutation in TP53 modified the expression of 892 genes (compared to CDKN2A^{mut} keratinocytes, adjusted p-value <0.05, at least twofold), two-thirds of which showed downregulation. GO term analysis showed a stronger activation of genes promoting the cell cycle, a decline of pro-apoptotic factors (BAX) and a downregulation of keratinocyte differentiation markers (IVL, FLG). These findings suggest that the combination of mutations in CDKN2A and TP53 is sufficient to force keratinocytes into a basal-like state. The additional mutation in NOTCH1 resulted in changes in expression of 1,020 genes (compared to CT^{mut} keratinocytes, adjusted P value <0.05, at least twofold). GO term analysis of this set of genes showed the upregulation of anti-apoptotic factors and basal-keratinocyte markers (KRT14), and a shift in the expression of adhesion molecules (ITGA1, ITGA6, ICAM1). Finally, NOTCH1 mutation in this background induces an EMT-like program that includes the downregulation of E-cadherin (CDH1) and activation of N-cadherin (CDH2) (FIG. 25B and FIG. 25C, FIG. 26, Table 8). These data, along with our organotypic cultures and in vivo data, indicate that combined mutations in CDKN2A, TP53, and NOTCH1 are sufficient to transform human primary keratinocytes into cancer and is accompanied by an induction of cell-cycle progression, a block of keratinocyte differentiation, and induction of an EMT-like phenotype.

TABLE 8
Results of Differential Gene Expression Analysis.

| baseMean | log2FoldChange | lfcSE | stat | pvalue | padj | hgnc_symbol |
|---|---|---|---|---|---|---|
| 193.2552834 | 5.455822457 | 0.275521445 | 19.80180695 | 2.87E-87 | 2.14E-84 | CXCL5 |
| 119.5534697 | -5.437311242 | 0.307263725 | -17.69591008 | 4.51E-70 | 2.17E-67 | HLA-DQB1 |
| 232.6366223 | 5.144915519 | 0.2511104 | 20.48865964 | 2.72E-93 | 2.47E-90 | MMP10 |
| 317.9092607 | 5.104955547 | 0.211758096 | 24.10748698 | 2.09E-128 | 8.55E-125 | NOS1 |
| 326.8426926 | 4.981751032 | 0.210310031 | 23.68765298 | 4.83E-124 | 1.32E-120 | SOX11 |
| 170.2869601 | 4.293982989 | 0.232443098 | 18.47326519 | 3.39E-76 | 2.06E-73 | POSTN |
| 85.9456744 | -4.244225457 | 0.280903645 | -15.10918611 | 1.41E-51 | 3.25E-49 | CTSH |
| 205.5016868 | 4.144116058 | 0.206338861 | 20.08403086 | 1.02E-89 | 7.94E-87 | MMP9 |
| 45.05689572 | 4.052548086 | 0.335890761 | 12.06507758 | 1.62E-33 | 1.58E-31 | PRSS21 |
| 65.78797665 | 4.033267838 | 0.303225937 | 13.30119674 | 2.28E-40 | 3.11E-38 | COX20P1 |
| 75.06419412 | 3.980568523 | 0.288231722 | 13.81030685 | 2.21E-43 | 3.45E-41 | SLC10A6 |
| 94.4419079 | -3.807459634 | 0.254898578 | -14.93715523 | 1.89E-50 | 4.18E-48 | LINC01116 |
| 39.8848753 | 3.708290877 | 0.333774268 | 11.11017604 | 1.12E-28 | 8.41E-27 | IRX1 |
| 68.2966123 | -3.662958176 | 0.279872441 | -13.08795595 | 3.86E-39 | 5.02E-37 | CYBA |
| 119.3423637 | 3.661589119 | 0.234794175 | 15.59488907 | 7.89E-55 | 2.08E-52 | STEAP1B |
| 121.6866391 | -3.628795263 | 0.225262189 | -16.10920711 | 2.20E-58 | 6.55E-56 | FOXA2 |
| 34.47239949 | 3.597215399 | 0.341582928 | 10.53101636 | 6.22E-26 | 3.96E-24 | CCDC152 |
| 31.17675173 | 3.575869922 | 0.346332214 | 10.32497057 | 5.43E-25 | 3.27E-23 | ESM1 |
| 32.3719363 | 3.557448277 | 0.341315331 | 10.42276145 | 1.95E-25 | 1.18E-23 | FAM155B |
| 44.13309552 | 3.548420657 | 0.326538509 | 10.86677546 | 1.66E-27 | 1.16E-25 | LGALS7 |
| 51.44890307 | 3.507282521 | 0.303400148 | 11.55992358 | 6.58E-31 | 5.53E-29 | LINC00960 |
| 28.72141455 | 3.470609939 | 0.348509472 | 9.958437923 | 2.32E-23 | 1.29E-21 | LEF1 |
| 65.35262506 | -3.377708064 | 0.274067456 | -12.32436758 | 6.70E-35 | 6.94E-33 | SLFN11 |
| 562.5274469 | 3.361952194 | 0.146405427 | 22.96330314 | 1.09E-116 | 2.22E-113 | MMP1 |
| 150.2488859 | 3.349403495 | 0.207172079 | 16.16725337 | 8.58E-59 | 2.70E-56 | TNFSF15 |
| 138.2038405 | -3.30638207 | 0.204586075 | -16.16132508 | 9.45E-59 | 2.92E-56 | CLDN11 |
| 195.8720888 | 3.305217735 | 0.189296702 | 17.46051413 | 2.86E-68 | 1.20E-65 | ERAP2 |
| 49.26922676 | 3.270132562 | 0.305611048 | 10.70030871 | 1.01E-26 | 6.78E-25 | TUBBP5 |
| 149.907814 | -3.172460921 | 0.197081933 | -16.09716764 | 2.67E-58 | 7.81E-56 | TMEM204 |
| 136.0541136 | -3.162250264 | 0.204822872 | -15.43895093 | 8.95E-54 | 2.19E-51 | CSF3 |
| 20.59896487 | -3.145677503 | 0.355970379 | -8.836908037 | 9.84E-19 | 4.02E-17 | PSG4 |
| 70.68601126 | 3.080107509 | 0.261329959 | 11.78627784 | 4.59E-32 | 4.05E-30 | FGFR1 |
| 20.71712319 | -3.066028997 | 0.353603122 | -8.670819931 | 4.29E-18 | 1.71E-16 | MTND4P24 |
| 22.61624969 | 3.055359717 | 0.353034417 | 8.654566155 | 4.95E-18 | 1.96E-16 | EEF1GPI |
| 61.9830611 | 3.004836719 | 0.269773633 | 11.13836326 | 8.16E-29 | 6.22E-27 | CPPED1 |
| 59.06606914 | -2.993291106 | 0.270674916 | -11.05862024 | 1.99E-28 | 1.47E-26 | PNMA8A |
| 18.5786669 | -2.96567328 | 0.359531553 | -8.248714902 | 1.60E-16 | 5.64E-15 | NA |
| 20.21113501 | 2.942485776 | 0.354782711 | 8.293768784 | 1.10E-16 | 3.90E-15 | TTN-AS1 |
| 35.93213777 | 2.939208607 | 0.315203281 | 9.32480335 | 1.11E-20 | 5.28E-19 | NDN |
| 18.87734182 | 2.929511337 | 0.358894666 | 8.162593692 | 3.28E-16 | 1.13E-14 | ZNF280B |
| 37.02685519 | 2.91968605 | 0.320566772 | 9.10788737 | 8.40E-20 | 3.69E-18 | IGFL3 |
| 74.83313552 | 2.871364637 | 0.248516563 | 11.55401717 | 7.04E-31 | 5.86E-29 | RASGEF1A |
| 20.2206575 | 2.845879851 | 0.352738767 | 8.067953166 | 7.15E-16 | 2.39E-14 | FAM180A |
| 40.55482875 | -2.820908066 | 0.303245068 | -9.302403788 | 1.37E-20 | 6.50E-19 | HOXB4 |
| 73.65560894 | 2.802847632 | 0.253876137 | 11.04021696 | 2.44E-28 | 1.80E-26 | SCN8A |
| 18.40722129 | 2.801676489 | 0.357467248 | 7.83757535 | 4.59E-15 | 1.42E-13 | APCDD1 |
| 274.9302689 | -2.796172714 | 0.154717754 | -18.0727333 | 5.23E-73 | 2.68E-70 | CNTN1 |
| 63.0177826 | -2.79026694 | 0.25927966 | -10.76161137 | 5.22E-27 | 3.55E-25 | TRHDE-AS1 |
| 16.7332851 | 2.789531398 | 0.360993747 | 7.727367636 | 1.10E-14 | 3.28E-13 | LINC01835 |
| 30.66733182 | -2.743168831 | 0.322579598 | -8.503850975 | 1.83E-17 | 7.00E-16 | FAM198B- |
| | | | | | | AS1 |
| 82.48211968 | -2.741884315 | 0.237801692 | -11.53012956 | 9.30E-31 | 7.58E-29 | NTSR1 |
| 256.605606 | 2.739428025 | 0.171963116 | 15.93032325 | 3.90E-57 | 1.10E-54 | KRT75 |
| 133.7976973 | -2.736785133 | 0.20000764 | -13.68340294 | 1.28E-42 | 1.90E-40 | PINLYP |
| 2029.067072 | 2.734202558 | 0.09255845 | 29.54028039 | 8.75E-192 | 1.43E-187 | SPRR2D |
| 434.5634113 | -2.733645321 | 0.128691907 | -21.24178105 | 3.93E-100 | 4.95E-97 | MME |
| 19.487628 | -2.723311855 | 0.354836415 | -7.674837593 | 1.66E-14 | 4.88E-13 | TLR2 |
| 605.850793 | -2.713118168 | 0.114532868 | -23.68855523 | 4.73E-124 | 1.32E-120 | BST2 |
| 29.96136566 | -2.701553842 | 0.324083527 | -8.335980145 | 7.69E-17 | 2.77E-15 | TYWIB |
| 31.49047422 | 2.683267362 | 0.320801889 | 8.364250512 | 6.05E-17 | 2.20E-15 | LINC01322 |
| 15.19997056 | 2.678500627 | 0.362494161 | 7.389086274 | 1.48E-13 | 4.03E-12 | NAP1L2 |
| 16.7365735 | -2.666280869 | 0.357996209 | -7.447790789 | 9.49E-14 | 2.63E-12 | NA |
| 105.9101021 | 2.635387367 | 0.216040881 | 12.19855871 | 3.16E-34 | 3.24E-32 | SPINK6 |
| 14.85509634 | 2.631828016 | 0.36321616 | 7.245900119 | 4.30E-13 | 1.12E-11 | TNS1 |
| 47.41360713 | 2.626300022 | 0.284756293 | 9.222974478 | 2.89E-20 | 1.33E-18 | COL5A1 |
| 17.88633829 | -2.618691439 | 0.356440584 | -7.346782488 | 2.03E-13 | 5.48E-12 | DPYSL4 |
| 25.82810001 | -2.610143168 | 0.333836616 | -7.818624582 | 5.34E-15 | 1.64E-13 | PI15 |
| 37.67742299 | 2.582483611 | 0.308283094 | 8.376987442 | 5.43E-17 | 1.99E-15 | COL11A1 |
| 48.59944078 | 2.581612979 | 0.282001968 | 9.154592051 | 5.46E-20 | 2.44E-18 | APBA2 |
| 77.08575695 | -2.565440724 | 0.23164897 | -11.07469082 | 1.66E-28 | 1.23E-26 | CFH |
| 100.9275676 | 2.562438196 | 0.217764584 | 11.76701075 | 5.77E-32 | 5.03E-30 | CAPNS2 |
| 13.75352406 | 2.56164031 | 0.363903995 | 7.039330002 | 1.93E-12 | 4.77E-11 | KIAA1324L |
| 27.62244064 | 2.560487574 | 0.326327066 | 7.846384327 | 4.28E-15 | 1.33E-13 | TSPYL5 |
| 112.6127226 | 2.552388182 | 0.210573486 | 12.12112803 | 8.16E-34 | 8.10E-32 | SCG5 |
| 233.2355295 | 2.551500464 | 0.153179665 | 16.65691375 | 2.70E-62 | 9.60E-60 | RBP1 |
| 64.38105596 | -2.545067186 | 0.248291816 | -10.25030637 | 1.18E-24 | 7.03E-23 | TREM2 |
| 46.75753988 | -2.541791352 | 0.291201019 | -8.728648561 | 2.58E-18 | 1.04E-16 | LOXL2 |
| 13.65974313 | 2.524651752 | 0.364443876 | 6.9274089 | 4.29E-12 | 1.02E-10 | DACT2 |
| 66.43295129 | -2.512669783 | 0.246651292 | -10.18713406 | 2.26E-24 | 1.33E-22 | HCLS1 |
| 13.8386727 | -2.500794119 | 0.363323811 | -6.88309998 | 5.86E-12 | 1.37E-10 | NA |
| 96.48423891 | 2.496211243 | 0.220216135 | 11.33527863 | 8.78E-30 | 6.88E-28 | CXCL3 |
| 80.81679305 | -2.490941704 | 0.238628397 | -10.43858037 | 1.65E-25 | 1.02E-23 | HOXB7 |
| 20.36911674 | 2.479685789 | 0.346702098 | 7.152208783 | 8.54E-13 | 2.17E-11 | PCDHGA12 |
| 48.72701937 | -2.474322973 | 0.269364452 | -9.185781386 | 4.09E-20 | 1.85E-18 | ORC1 |
| 14.04194241 | 2.465904903 | 0.362897158 | 6.795051567 | 1.08E-11 | 2.46E-10 | PEG3 |
| 410.1386314 | -2.461857711 | 0.127342184 | -19.33261724 | 2.86E-83 | 1.95E-80 | PLIN2 |
| 208.1265004 | 2.461031345 | 0.159113903 | 15.46710438 | 5.79E-54 | 1.44E-51 | ABCG1 |
| 24.82019172 | 2.45430812 | 0.340159934 | 7.21515932 | 5.39E-13 | 1.39E-11 | TMPRSS11B |
| 181.160236 | 2.452772954 | 0.17630328 | 13.91223664 | 5.34E-44 | 8.57E-42 | ULBP1 |
| 49.99665332 | -2.440931154 | 0.268614017 | -9.087132471 | 1.02E-19 | 4.44E-18 | LAMA1 |
| 73.51892017 | 2.440408847 | 0.243088458 | 10.03918027 | 1.03E-23 | 5.79E-22 | CLIP3 |
| 23.24301771 | -2.428569394 | 0.334008474 | -7.270981383 | 3.57E-13 | 9.38E-12 | NA |
| 293.6154369 | 2.424161871 | 0.163295286 | 14.84526547 | 7.47E-50 | 1.57E-47 | KRT31 |
| 41.27560508 | -2.417675258 | 0.285533607 | -8.467217859 | 2.51E-17 | 9.44E-16 | TRHDE |
| 179.4934952 | 2.415938056 | 0.171170833 | 14.11419231 | 3.11E-45 | 5.41E-43 | KRT34 |
| 246.7120125 | 2.406882043 | 0.153474872 | 15.68258054 | 1.99E-55 | 5.43E-53 | KRTAP2-3 |
| 264.5492841 | -2.387294896 | 0.155524872 | -15.34992355 | 3.55E-53 | 8.54E-51 | DTL |
| 14.45865876 | 2.385130954 | 0.361304793 | 6.601437349 | 4.07E-11 | 8.54E-10 | VLDLR-AS1 |
| 31.03935143 | -2.366616631 | 0.316325985 | -7.481575149 | 7.34E-14 | 2.06E-12 | TWIST1 |
| 136.8287767 | 2.364297739 | 0.191059492 | 12.37466779 | 3.58E-35 | 3.76E-33 | NA |
| 40.84692217 | 2.363692423 | 0.291523353 | 8.108072308 | 5.14E-16 | 1.75E-14 | NEGR1 |
| 11.62133441 | -2.346640227 | 0.366337783 | -6.405673492 | 1.50E-10 | 2.92E-09 | MTCYBP18 |
| 715.1936794 | 2.345087572 | 0.116428945 | 20.14179178 | 3.18E-90 | 2.60E-87 | SPRR2A |
| 11.27150601 | 2.302071121 | 0.366167499 | 6.28693461 | 3.24E-10 | 6.12E-09 | NA |
| 180.4522163 | 2.294152489 | 0.17382784 | 13.1978427 | 9.03E-40 | 1.19E-37 | ADM2 |
| 79.85794551 | 2.293746657 | 0.23897686 | 9.598195634 | 8.14E-22 | 4.14E-20 | MEIS3 |
| 19.81722945 | 2.292773692 | 0.344577626 | 6.653866992 | 2.85E-11 | 6.08E-10 | SYNGR3 |
| 12.73796574 | -2.289669182 | 0.36391529 | -6.291764175 | 3.14E-10 | 5.97E-09 | HOXB3 |
| 22.72718407 | 2.281862186 | 0.334994498 | 6.811640783 | 9.65E-12 | 2.21E-10 | HLA-G |
| 127.8863812 | 2.273236999 | 0.341663017 | 6.653447655 | 2.86E-11 | 6.09E-10 | SPRR2G |
| 412.8061047 | -2.270491722 | 0.122569168 | -18.52416688 | 1.32E-76 | 8.64E-74 | ELP5 |
| 51.99990918 | 2.267050982 | 0.267387623 | 8.478518768 | 2.28E-17 | 8.63E-16 | BEND7 |
| 288.5597217 | -2.259611424 | 0.142490381 | -15.85799273 | 1.24E-56 | 3.44E-54 | MCM10 |
| 13.20253984 | -2.258151995 | 0.3621325 | -6.235706525 | 4.50E-10 | 8.34E-09 | HERC2P3 |
| 95.89548054 | 2.252211295 | 0.220162942 | 10.22974744 | 1.46E-24 | 8.66E-23 | PYGO1 |
| 73.80823575 | -2.248081273 | 0.232306178 | -9.677234123 | 3.77E-22 | 1.96E-20 | CCNE2 |
| 10.73462634 | 2.23561157 | 0.366470877 | 6.100379901 | 1.06E-09 | 1.89E-08 | SLC27A6 |
| 12.3856895 | 2.218865278 | 0.363543712 | 6.103434624 | 1.04E-09 | 1.86E-08 | BRSK1 |
| 288.986101 | -2.218303356 | 0.166446865 | -13.32739644 | 1.60E-40 | 2.21E-38 | SPRR3 |
| 427.9473485 | -2.211348475 | 0.122105868 | -18.11009171 | 2.65E-73 | 1.40E-70 | FAM111B |
| 217.2075559 | 2.205812354 | 0.185253015 | 11.90702542 | 1.09E-32 | 9.85E-31 | PRSS3 |
| 31.67238824 | 2.203978647 | 0.306599335 | 7.188465197 | 6.55E-13 | 1.68E-11 | RTN4RL1 |
| 39.6965331 | -2.197407295 | 0.287320341 | -7.647935005 | 2.04E-14 | 5.95E-13 | SAA2 |
| 23.17833281 | -2.194676146 | 0.335992105 | -6.531927734 | 6.49E-11 | 1.32E-09 | CX3CL1 |
| 26.42856356 | -2.187032996 | 0.322670706 | -6.777909974 | 1.22E-11 | 2.74E-10 | SLFN13 |
| 202.4481448 | 2.180193528 | 0.160378078 | 13.59408688 | 4.34E-42 | 6.35E-40 | IRX3 |
| 25.48879009 | 2.174395973 | 0.330767824 | 6.573783226 | 4.91E-11 | 1.01E-09 | ICA1 |
| 179.1168414 | -2.151952109 | 0.161188889 | -13.35049901 | 1.18E-40 | 1.65E-38 | CSPG4 |
| 26.51078412 | -2.147327677 | 0.328035123 | -6.546029765 | 5.91E-11 | 1.21E-09 | DISP2 |
| 193.4701371 | -2.146103148 | 0.171864008 | -12.4872169 | 8.77E-36 | 9.70E-34 | MTND4P12 |
| 225.0421525 | -2.14107858 | 0.15631532 | -13.69717687 | 1.06E-42 | 1.59E-40 | EPSTI1 |
| 44.65189814 | 2.137580686 | 0.273693692 | 7.8101204 | 5.71E-15 | 1.75E-13 | ZFP57 |
| 25.59630038 | 2.13704612 | 0.330965567 | 6.457004392 | 1.07E-10 | 2.13E-09 | BEX5 |
| 80.64154665 | -2.133347352 | 0.232659656 | -9.169390986 | 4.76E-20 | 2.14E-18 | NRGN |
| 128.1639336 | -2.133153944 | 0.184877469 | -11.53820395 | 8.47E-31 | 6.94E-29 | SYK |
| 35.42764864 | -2.133019063 | 0.293985105 | -7.255534459 | 4.00E-13 | 1.05E-11 | SLC15A3 |
| 636.905113 | -2.129054516 | 0.105308615 | -20.21728731 | 6.90E-91 | 5.95E-88 | PCLAF |
| 711.2025111 | -2.118304009 | 0.10306493 | -20.55310189 | 7.22E-94 | 6.96E-91 | NCAPG |
| 10.52281001 | 2.117947661 | 0.366079052 | 5.785492647 | 7.23E-09 | 1.19E-07 | C6orf223 |
| 20.48244389 | 2.112016072 | 0.340553 | 6.201725052 | 5.58E-10 | 1.03E-08 | TBX1 |
| 56.67442884 | -2.109680906 | 0.250657984 | -8.416571733 | 3.88E-17 | 1.43E-15 | FAM198B |
| 342.8251762 | 2.105341552 | 0.127668871 | 16.49064131 | 4.28E-61 | 1.46E-58 | ALDH1L2 |
| 14.4837212 | -2.103330547 | 0.357987185 | -5.875435314 | 4.22E-09 | 7.08E-08 | NLRP3 |
| 19.42518485 | -2.100790598 | 0.340585514 | -6.16817366 | 6.91E-10 | 1.26E-08 | ANKUB1 |
| 11.04067014 | -2.087165175 | 0.365843244 | -5.705080552 | 1.16E-08 | 1.84E-07 | CASP14 |
| 12.38829908 | 2.087041578 | 0.36336423 | 5.743662712 | 9.27E-09 | 1.49E-07 | ANKRD18CP |
| 1484.383123 | 2.083809799 | 0.093092535 | 22.38428464 | 5.60E-111 | 1.02E-107 | PTGS2 |
| 85.81359762 | 2.081296172 | 0.222852648 | 9.339337855 | 9.69E-21 | 4.62E-19 | LY6D |
| 27.08055104 | -2.075914395 | 0.319607089 | -6.495207608 | 8.29E-11 | 1.67E-09 | RNY1 |
| 33.48038568 | -2.072708984 | 0.306861072 | -6.754551727 | 1.43E-11 | 3.18E-10 | METTL7A |
| 13.34851317 | 2.068195494 | 0.361631184 | 5.719073975 | 1.07E-08 | 1.71E-07 | CA5BP1 |
| 37.72604575 | 2.062812159 | 0.287962921 | 7.163464494 | 7.87E-13 | 2.00E-11 | PTPRS |
| 13.83290791 | -2.05589431 | 0.35853525 | -5.734148348 | 9.80E-09 | 1.57E-07 | MYBPHL |
| 1095.114242 | 2.046349103 | 0.257742865 | 7.939498567 | 2.03E-15 | 6.55E-14 | KRT16P6 |
| 10.40339263 | -2.042791655 | 0.366070168 | -5.58032812 | 2.40E-08 | 3.63E-07 | AKNAD1 |
| 26.14922077 | -2.041899593 | 0.319138323 | -6.398164824 | 1.57E-10 | 3.06E-09 | PTMAP9 |
| 184.8756643 | -2.038474815 | 0.158028259 | -12.89943224 | 4.53E-38 | 5.63E-36 | ELAVL2 |
| 16.79308946 | 2.036262138 | 0.352172388 | 5.782003945 | 7.38E-09 | 1.21E-07 | ADAM23 |
| 69.66970036 | -2.034125588 | 0.236240922 | -8.610386276 | 7.28E-18 | 2.85E-16 | ZNF367 |
| 265.3695946 | -2.033113631 | 0.136433733 | -14.90183978 | 3.21E-50 | 6.82E-48 | CDT1 |
| 10.85696899 | 2.032505458 | 0.365608103 | 5.559246199 | 2.71E-08 | 4.07E-07 | CCDC170 |
| 94.66243915 | 2.032455792 | 0.214929209 | 9.456396385 | 3.19E-21 | 1.56E-19 | SV2A |
| 80.27223865 | -2.027979957 | 0.223592397 | -9.06998622 | 1.19E-19 | 5.17E-18 | ERCC6L |
| 75.23335273 | -2.022451081 | 0.22470974 | -9.00028223 | 2.25E-19 | 9.58E-18 | PTGS1 |
| 141.7935608 | 2.021300895 | 0.339632089 | 5.951442628 | 2.66E-09 | 4.57E-08 | CDSN |
| 60.51209378 | 2.018977471 | 0.254399369 | 7.93625187 | 2.08E-15 | 6.69E-14 | SLC9A3-AS1 |
| 194.0344515 | 2.016811912 | 0.160269717 | 12.58386146 | 2.59E-36 | 2.95E-34 | CWH43 |
| 82.38987592 | 2.015669317 | 0.218338258 | 9.231864962 | 2.66E-20 | 1.24E-18 | PLEKHG4 |
| 10.66648343 | 2.013504988 | 0.365618509 | 5.507119959 | 3.65E-08 | 5.40E-07 | LINC00958 |
| 66.04695737 | -2.002933895 | 0.244702765 | -8.185170662 | 2.72E-16 | 9.44E-15 | C17orf97 |
| 165.9991954 | 2.002252282 | 0.164898316 | 12.14234523 | 6.30E-34 | 6.29E-32 | CXCL1 |
| 156.1368395 | -1.999614636 | 0.168554937 | -11.86328133 | 1.84E-32 | 1.64E-30 | GDF15 |
| 8.900168294 | 1.993533318 | 0.366304517 | 5.442284287 | 5.26E-08 | 7.63E-07 | PSPHP1 |
| 66.96724802 | 1.992932724 | 0.239017756 | 8.338011198 | 7.56E-17 | 2.73E-15 | RORA |
| 48.5247778 | -1.992407638 | 0.27938349 | -7.131443735 | 9.93E-13 | 2.50E-11 | PRR9 |
| 253.3546581 | -1.990842971 | 0.142628932 | -13.9581987 | 2.80E-44 | 4.64E-42 | CDC6 |
| 28.00322572 | 1.98441396 | 0.31120878 | 6.376471645 | 1.81E-10 | 3.51E-09 | ASPHD1 |
| 199.4477413 | 1.983964057 | 0.169128092 | 11.73054126 | 8.89E-32 | 7.62E-30 | ASS1 |
| 163.8429283 | -1.979580617 | 0.165909673 | -11.93167692 | 8.09E-33 | 7.45E-31 | CDCA5 |
| 1065.197846 | -1.975099139 | 0.089571255 | -22.05059136 | 9.43E-108 | 1.54E-104 | ANLN |
| 931.046002 | -1.959881892 | 0.093885653 | -20.87520124 | 9.00E-97 | 9.83E-94 | NEFL |
| 31.48860984 | 1.959679238 | 0.306770326 | 6.388099088 | 1.68E-10 | 3.26E-09 | EMX20S |
| 9.063336688 | 1.951716982 | 0.36665001 | 5.323106308 | 1.02E-07 | 1.42E-06 | NA |
| 24.42742652 | -1.949492222 | 0.328517101 | -5.934218392 | 2.95E-09 | 5.05E-08 | CCDC144B |
| 377.588117 | 1.94945331 | 0.147457425 | 13.22044864 | 6.69E-40 | 8.91E-38 | KRT6C |
| 154.3165641 | -1.949452443 | 0.185991882 | -10.48138459 | 1.05E-25 | 6.58E-24 | FOS |
| 181.6961861 | -1.945839017 | 0.173393674 | -11.2220877 | 3.18E-29 | 2.44E-27 | ESCO2 |
| 11.7038359 | -1.942363097 | 0.364986036 | -5.321746327 | 1.03E-07 | 1.42E-06 | DEPP1 |
| 1103.984753 | -1.940610866 | 0.094167881 | -20.60799131 | 2.33E-94 | 2.38E-91 | SUGCT |
| 83.03286734 | -1.939582637 | 0.213055101 | -9.103666742 | 8.73E-20 | 3.83E-18 | CCNJL |
| 50.57339379 | -1.935126945 | 0.270047485 | -7.165876557 | 7.73E-13 | 1.97E-11 | VNN1 |
| 44.97779595 | 1.928912881 | 0.276955444 | 6.964704691 | 3.29E-12 | 7.93E-11 | PAPPA2 |
| 65.52225402 | -1.927655993 | 0.231480901 | -8.327494753 | 8.26E-17 | 2.96E-15 | EXO1 |
| 235.5065066 | -1.927165538 | 0.154547106 | -12.46976141 | 1.09E-35 | 1.20E-33 | ASF1B |
| 185.1118597 | -1.924872803 | 0.163322858 | -11.78569142 | 4.63E-32 | 4.05E-30 | FAM83D |
| 9.414814331 | 1.922342762 | 0.366629409 | 5.243285774 | 1.58E-07 | 2.12E-06 | NA |
| 24.41812926 | -1.921776217 | 0.324905507 | -5.914877318 | 3.32E-09 | 5.66E-08 | SP140 |
| 14.85408028 | 1.921018519 | 0.357406937 | 5.374877541 | 7.66E-08 | 1.09E-06 | TDRD12 |
| 172.4035624 | -1.920416909 | 0.173003069 | -11.10047888 | 1.25E-28 | 9.33E-27 | GINS1 |
| 338.0499431 | -1.919061781 | 0.137604187 | -13.94624559 | 3.32E-44 | 5.43E-42 | HJURP |
| 193.149451 | 1.916093155 | 0.182454888 | 10.50173651 | 8.48E-26 | 5.34E-24 | SDCBP2 |
| 25.90263752 | -1.911119595 | 0.313346722 | -6.09905725 | 1.07E-09 | 1.90E-08 | PLCL2 |
| 768.3692384 | -1.907297756 | 0.104774804 | -18.20378268 | 4.82E-74 | 2.63E-71 | CDC20 |
| 8.774476701 | 1.904611765 | 0.366625323 | 5.194981483 | 2.05E-07 | 2.71E-06 | ZNF736P9Y |
| 164.6713754 | -1.903636314 | 0.161985414 | -11.75189954 | 6.90E-32 | 5.99E-30 | NCAPH |
| 143.5156817 | -1.902780449 | 0.173532553 | -10.964977 | 5.63E-28 | 4.06E-26 | ZFP42 |
| 8.934247452 | 1.899965006 | 0.366595881 | 5.182723281 | 2.19E-07 | 2.88E-06 | PLBD1 |
| 136.3996527 | -1.899869328 | 0.176983595 | -10.73471997 | 6.99E-27 | 4.71E-25 | GINS2 |
| 95.25373554 | -1.898605987 | 0.203252067 | -9.341139876 | 9.53E-21 | 4.55E-19 | HSD17B2 |
| 76.36736733 | -1.897590271 | 0.223331254 | -8.496751946 | 1.95E-17 | 7.43E-16 | SPC24 |
| 430.8431599 | -1.895126472 | 0.114504647 | -16.55065123 | 1.58E-61 | 5.52E-59 | RRM2 |
| 339.5493713 | -1.890739196 | 0.132233015 | -14.29854109 | 2.23E-46 | 3.94E-44 | P3H2 |
| 74.20684715 | -1.889697451 | 0.230743016 | -8.189619276 | 2.62E-16 | 9.12E-15 | CENPM |
| 56.94277123 | -1.889423126 | 0.24530085 | -7.702472803 | 1.33E-14 | 3.96E-13 | SCNNIA |
| 99.24366529 | 1.882013921 | 0.208962964 | 9.006447288 | 2.13E-19 | 9.13E-18 | LURAP1L- |
| | | | | | | AS1 |
| 514.5206285 | -1.88003596 | 0.111554492 | -16.85307266 | 9.96E-64 | 3.89E-61 | CLSPN |
| 100.6868883 | 1.879260023 | 0.217904224 | 8.6242478 | 6.45E-18 | 2.54E-16 | EMX2 |
| 142.8285935 | 1.871287362 | 0.176878053 | 10.57953393 | 3.71E-26 | 2.38E-24 | SYT7 |
| 38.96160171 | -1.868392024 | 0.306257588 | -6.100720753 | 1.06E-09 | 1.88E-08 | KIF18B |
| 210.5769866 | -1.863186718 | 0.153208175 | -12.16114429 | 5.01E-34 | 5.03E-32 | SHCBP1 |
| 42.92831513 | 1.862599661 | 0.275499114 | 6.760819054 | 1.37E-11 | 3.06E-10 | NKAPL |
| 247.8056199 | 1.859905624 | 0.155510337 | 11.9600128 | 5.76E-33 | 5.42E-31 | SEZ6L2 |
| 131.3775475 | -1.858062876 | 0.185057618 | -10.04045601 | 1.01E-23 | 5.74E-22 | CDCA3 |
| 14.23054545 | -1.856101118 | 0.355290052 | -5.224185439 | 1.75E-07 | 2.34E-06 | BDNF |
| 1768.72627 | -1.849394031 | 0.078130543 | -23.67056419 | 7.25E-124 | 1.70E-120 | PRELID1 |
| 11.44280892 | 1.847381194 | 0.364574278 | 5.067228554 | 4.04E-07 | 5.08E-06 | NA |
| 39.82877043 | 1.846596149 | 0.291078917 | 6.343970794 | 2.24E-10 | 4.32E-09 | DUSP8 |
| 117.320078 | -1.842115492 | 0.18620591 | -9.892894888 | 4.47E-23 | 2.44E-21 | FOXM1 |
| 66.7406101 | -1.839713104 | 0.229640372 | -8.011279073 | 1.14E-15 | 3.74E-14 | APOL1 |
| 242.0767963 | -1.829614835 | 0.151134376 | -12.10588142 | 9.83E-34 | 9.70E-32 | LMNB1 |
| 340.3973373 | -1.829565881 | 0.124562329 | -14.68795494 | 7.70E-49 | 1.56E-46 | CCNA2 |
| 245.0055496 | -1.823088029 | 0.146275815 | -12.4633592 | 1.18E-35 | 1.29E-33 | MYBL2 |
| 48.73892293 | -1.81440256 | 0.288559819 | -6.287786599 | 3.22E-10 | 6.09E-09 | HIST1H4H |
| 600.7276182 | -1.814028339 | 0.10335504 | -17.55142598 | 5.80E-69 | 2.72E-66 | CDK1 |
| 1504.818078 | -1.81375784 | 0.084308993 | -21.51321902 | 1.17E-102 | 1.74E-99 | LCP1 |
| 3615.172019 | -1.805539892 | 0.073501747 | -24.56458464 | 3.02E-133 | 1.65E-129 | TOP2A |
| 18.40398949 | 1.803243465 | 0.338585282 | 5.325817637 | 1.00E-07 | 1.40E-06 | LY96 |
| 28.65606134 | -1.802871546 | 0.305041175 | -5.910256361 | 3.42E-09 | 5.79E-08 | MCHR1 |
| 10.54527554 | 1.798816699 | 0.365136189 | 4.926426778 | 8.37E-07 | 1.00E-05 | NA |
| 9.982730924 | -1.798368886 | 0.365687014 | -4.917781644 | 8.75E-07 | 1.04E-05 | SERPINA1 |
| 15.04824786 | -1.796433467 | 0.351449499 | -5.111498158 | 3.20E-07 | 4.09E-06 | HIST1H2AH |
| 75.3017071 | 1.793665922 | 0.223232181 | 8.034979156 | 9.36E-16 | 3.10E-14 | DNAJA4 |
| 504.2376992 | -1.789033567 | 0.109124348 | -16.39444904 | 2.10E-60 | 6.87E-58 | BIRC5 |
| 3156.829309 | -1.788086201 | 0.071601748 | -24.97266132 | 1.21E-137 | 9.93E-134 | KRT7 |
| 177.6825961 | -1.785929712 | 0.160619393 | -11.11901673 | 1.01E-28 | 7.69E-27 | KITLG |
| 11.40171073 | 1.785765576 | 0.363616932 | 4.911117769 | 9.06E-07 | 1.08E-05 | VWDE |
| 10.02253758 | -1.783292027 | 0.366026393 | -4.87203126 | 1.10E-06 | 1.29E-05 | NA |
| 1679.648502 | -1.77051668 | 0.095792087 | -18.48291166 | 2.83E-76 | 1.79E-73 | CENPF |
| 96.28063492 | -1.768245425 | 0.201775076 | -8.763448199 | 1.89E-18 | 7.70E-17 | CDC45 |
| 171.0164223 | 1.766585128 | 0.202470277 | 8.725157846 | 2.66E-18 | 1.07E-16 | CNFN |
| 62.96491811 | 1.764150355 | 0.241141376 | 7.315834336 | 2.56E-13 | 6.81E-12 | CTH |
| 81.77023419 | -1.762943125 | 0.212221719 | -8.307081543 | 9.81E-17 | 3.50E-15 | GINS4 |
| 8.222208269 | -1.762637055 | 0.366523857 | -4.809065004 | 1.52E-06 | 1.72E-05 | RNU4-2 |
| 219.7537201 | -1.755986827 | 0.148698774 | -11.80902025 | 3.51E-32 | 3.11E-30 | RAD51AP1 |
| 408.5829967 | -1.754329474 | 0.134586432 | -13.0349653 | 7.74E-39 | 9.98E-37 | PLK1 |
| 1539.617129 | -1.753310348 | 0.095218641 | -18.41352005 | 1.02E-75 | 5.99E-73 | MKI67 |
| 327.2182625 | -1.753261154 | 0.125946112 | -13.92072475 | 4.74E-44 | 7.69E-42 | SNHG17 |
| 78.65219902 | -1.751037358 | 0.216869719 | -8.074144081 | 6.80E-16 | 2.27E-14 | SPC25 |
| 27.8046681 | 1.75036424 | 0.306197393 | 5.716457032 | 1.09E-08 | 1.73E-07 | ZPLD1 |
| 112.0155154 | 1.742008882 | 0.187671381 | 9.282229804 | 1.66E-20 | 7.83E-19 | HOMER2 |
| 6.859113336 | 1.73856748 | 0.363803227 | 4.778867664 | 1.76E-06 | 1.99E-05 | COL4A4 |
| 12.27708444 | 1.738034251 | 0.359788025 | 4.830717346 | 1.36E-06 | 1.57E-05 | MEG3 |
| 233.7200874 | -1.735574182 | 0.152440123 | -11.38528455 | 4.95E-30 | 3.92E-28 | PBK |
| 25.91878693 | 1.734180935 | 0.319992375 | 5.419444556 | 5.98E-08 | 8.58E-07 | PPP2R2C |
| 773.7540942 | -1.734177042 | 0.099599473 | -17.41150818 | 6.75E-68 | 2.76E-65 | HELLS |
| 8.65356821 | -1.732325129 | 0.366629491 | -4.725002143 | 2.30E-06 | 2.54E-05 | HOXB5 |
| 91.84701788 | 1.73009621 | 0.21036844 | 8.224124335 | 1.97E-16 | 6.90E-15 | SMIM22 |
| 21.00011882 | 1.725556217 | 0.334059933 | 5.165409092 | 2.40E-07 | 3.14E-06 | PKN1 |
| 75.60006131 | 1.722892715 | 0.221368979 | 7.782900417 | 7.09E-15 | 2.15E-13 | ATP9A |
| 192.0767016 | -1.722718104 | 0.157589865 | -10.93165544 | 8.14E-28 | 5.85E-26 | FBXO5 |
| 286.8955385 | -1.717099819 | 0.133078148 | -12.90294342 | 4.33E-38 | 5.42E-36 | NDC80 |
| 74.56351907 | -1.71560459 | 0.230022714 | -7.458413812 | 8.76E-14 | 2.44E-12 | E2F1 |
| 175.1297453 | -1.71363848 | 0.1641698 | -10.43820772 | 1.66E-25 | 1.02E-23 | GTSE1 |
| 7.167112884 | -1.713524047 | 0.365050161 | -4.693941349 | 2.68E-06 | 2.91E-05 | FYB1 |
| 12.91488155 | -1.705411586 | 0.366197553 | -4.657080783 | 3.21E-06 | 3.44E-05 | LGALS7B |
| 138.1198568 | -1.705160191 | 0.207439083 | -8.220052691 | 2.03E-16 | 7.12E-15 | HIST1H1B |
| 415.3741229 | -1.703739718 | 0.121873689 | -13.97955314 | 2.08E-44 | 3.47E-42 | CKAP2L |
| 39.33083316 | 1.70140069 | 0.27879602 | 6.102672099 | 1.04E-09 | 1.86E-08 | HAS2 |
| 14.06830212 | 1.698813262 | 0.352946912 | 4.813226022 | 1.49E-06 | 1.69E-05 | FOXP2 |
| 243.634594 | -1.697899741 | 0.146261325 | -11.60867192 | 3.72E-31 | 3.14E-29 | MAD2L1 |
| 151.9144235 | 1.697628033 | 0.168198125 | 10.09302591 | 5.93E-24 | 3.41E-22 | FUOM |
| 9.474307334 | -1.695775682 | 0.366490563 | -4.627065075 | 3.71E-06 | 3.91E-05 | NA |
| 51.22477369 | -1.693492093 | 0.248415552 | -6.817174203 | 9.28E-12 | 2.13E-10 | NHLH2 |
| 35.11079242 | -1.690232214 | 0.291371438 | -5.800953677 | 6.59E-09 | 1.09E-07 | KCNS3 |
| 158.9901536 | -1.690165534 | 0.161940034 | -10.43698392 | 1.68E-25 | 1.03E-23 | SGO1 |
| 17.94383308 | 1.689187027 | 0.339579014 | 4.974356359 | 6.55E-07 | 7.96E-06 | TRPV4 |
| 375.8857964 | -1.688793827 | 0.124625621 | -13.55093615 | 7.82E-42 | 1.13E-39 | BRCA2 |
| 21.27440492 | -1.687557038 | 0.327812297 | -5.147936955 | 2.63E-07 | 3.43E-06 | HIST1H1A |
| 48.18149313 | -1.685190155 | 0.259631952 | -6.490688626 | 8.54E-11 | 1.72E-09 | IL7R |
| 49.02715331 | 1.68479043 | 0.257620488 | 6.539815392 | 6.16E-11 | 1.26E-09 | DIRC3 |
| 347.0378107 | -1.684019028 | 0.122917767 | -13.70037109 | 1.01E-42 | 1.53E-40 | UBE2C |
| 6.587130911 | 1.683901558 | 0.36279606 | 4.641454922 | 3.46E-06 | 3.67E-05 | JAM3 |
| 31.97689246 | 1.680322483 | 0.29260527 | 5.742625491 | 9.32E-09 | 1.50E-07 | EPGN |
| 13.4447931 | 1.68015369 | 0.356526256 | 4.712566495 | 2.45E-06 | 2.68E-05 | BAIAP2L2 |
| 216.5579165 | -1.680046207 | 0.155930503 | -10.77432689 | 4.55E-27 | 3.12E-25 | DEPDC1 |
| 735.1356943 | -1.679923365 | 0.095899937 | -17.51746065 | 1.05E-68 | 4.80E-66 | MCM7 |
| 258.339914 | -1.678239452 | 0.135030164 | -12.42862632 | 1.83E-35 | 1.96E-33 | LIG1 |
| 1120.299638 | -1.6771485 | 0.085602401 | -19.59230669 | 1.80E-85 | 1.28E-82 | IFITM2 |
| 199.4635033 | -1.673715587 | 0.173051519 | -9.671776317 | 3.97E-22 | 2.06E-20 | KIFC1 |
| 25.23602888 | 1.667529917 | 0.322343452 | 5.173146546 | 2.30E-07 | 3.02E-06 | RAB6B |
| 24.58774715 | 1.666482189 | 0.323145702 | 5.157061283 | 2.51E-07 | 3.27E-06 | DSG1 |
| 16.65249259 | -1.660929241 | 0.347120933 | -4.784872017 | 1.71E-06 | 1.93E-05 | HIST1H2BF |
| 24.18580892 | 1.653431659 | 0.32312796 | 5.116956322 | 3.11E-07 | 3.99E-06 | SHC2 |
| 257.2306367 | -1.652410593 | 0.157948433 | -10.4617093 | 1.29E-25 | 8.04E-24 | ATAD5 |
| 34.88957264 | -1.650145645 | 0.29023223 | -5.685604397 | 1.30E-08 | 2.05E-07 | SCN2A |
| 128.4388137 | -1.64899741 | 0.184725094 | -8.926764501 | 4.39E-19 | 1.83E-17 | KIF20A |
| 8.007359875 | -1.646420684 | 0.366623686 | -4.490764631 | 7.10E-06 | 7.10E-05 | RNY4 |
| 2182.56758 | -1.644388446 | 0.101525414 | -16.19681598 | 5.31E-59 | 1.71E-56 | RPL22L1 |
| 300.0836708 | -1.643618309 | 0.127668513 | -12.87410864 | 6.30E-38 | 7.70E-36 | PKMYT1 |
| 367.6372256 | 1.642373219 | 0.267015998 | 6.150842009 | 7.71E-10 | 1.40E-08 | SPRR2E |
| 665.5254046 | -1.642170171 | 0.112032019 | -14.65804313 | 1.20E-48 | 2.39E-46 | TMEM97 |
| 7.190963839 | 1.642105241 | 0.364877516 | 4.500428693 | 6.78E-06 | 6.83E-05 | HOXC12 |
| 6.272949121 | 1.641957626 | 0.36200236 | 4.53576498 | 5.74E-06 | 5.84E-05 | LINC01806 |
| 54.47482918 | -1.63950987 | 0.249993018 | -6.558222637 | 5.45E-11 | 1.12E-09 | VSTM2L |
| 514.9769983 | -1.638584262 | 0.118936675 | -13.7769469 | 3.51E-43 | 5.37E-41 | MOK |
| 1032.046494 | 1.638576 | 0.097478807 | 16.80956151 | 2.08E-63 | 7.91E-61 | TSPAN1 |
| 17.45523461 | -1.63480172 | 0.341665202 | -4.784806028 | 1.71E-06 | 1.93E-05 | FAXDC2 |
| 52.03161808 | -1.633810033 | 0.265014672 | -6.164979543 | 7.05E-10 | 1.28E-08 | ACSS1 |
| 337.535724 | -1.631097151 | 0.121000261 | -13.48011266 | 2.05E-41 | 2.92E-39 | DLGAP5 |
| 294.1759844 | 1.62665707 | 0.128273616 | 12.68115079 | 7.52E-37 | 8.87E-35 | LARGE2 |
| 6.292900294 | 1.626427356 | 0.361554967 | 4.498423491 | 6.85E-06 | 6.89E-05 | PCYT1B |
| 6.543400199 | 1.625593357 | 0.361267245 | 4.499697607 | 6.81E-06 | 6.85E-05 | TSKS |
| 181.61521 | -1.618956773 | 0.163648104 | -9.892914939 | 4.47E-23 | 2.44E-21 | KIF2C |
| 39.69708567 | -1.617184393 | 0.274852961 | -5.883816515 | 4.01E-09 | 6.76E-08 | ZNF85 |
| 28.53287794 | -1.616443581 | 0.309051332 | -5.230340117 | 1.69E-07 | 2.27E-06 | ARHGEF39 |
| 10.29214436 | -1.612269761 | 0.365503642 | -4.411090828 | 1.03E-05 | 9.87E-05 | FLI1 |
| 29.60322538 | -1.611245336 | 0.304939148 | -5.283825799 | 1.27E-07 | 1.73E-06 | FOXE1 |
| 418.4286795 | 1.608332844 | 0.128352633 | 12.53057932 | 5.08E-36 | 5.73E-34 | CHACI |
| 335.9609796 | -1.601673865 | 0.154026411 | -10.39869627 | 2.51E-25 | 1.52E-23 | CDCA8 |
| 111.7612165 | -1.598629599 | 0.191861922 | -8.332187954 | 7.94E-17 | 2.85E-15 | SERTAD4 |
| 1466.570029 | 1.597898386 | 0.105877431 | 15.09196407 | 1.83E-51 | 4.16E-49 | ADAMTS1 |
| 5.825359557 | -1.596976341 | 0.359604577 | -4.440923285 | 8.96E-06 | 8.71E-05 | ARL17B |
| 9.437142448 | -1.596893811 | 0.366300399 | -4.359519711 | 1.30E-05 | 0.000122378 | ADAMTS12 |
| 14.40237091 | -1.595021345 | 0.352765947 | -4.521471982 | 6.14E-06 | 6.22E-05 | NA |
| 763.7452248 | -1.593202742 | 0.095226295 | -16.73070177 | 7.83E-63 | 2.85E-60 | PCNA |
| 878.1750915 | 1.591499677 | 0.090987246 | 17.49145901 | 1.66E-68 | 7.37E-66 | LYPD3 |
| 17.61703887 | -1.588056728 | 0.342409569 | -4.637886523 | 3.52E-06 | 3.73E-05 | SNORD17 |
| 607.3518203 | -1.582092555 | 0.109739738 | -14.41676992 | 4.06E-47 | 7.47E-45 | TACC3 |
| 21.14248123 | -1.581728648 | 0.335321214 | -4.717055114 | 2.39E-06 | 2.63E-05 | ZNF732 |
| 5.793912915 | -1.580683281 | 0.3590842 | -4.401985056 | 1.07E-05 | 0.000102468 | HLA-DQB2 |
| 6.611233937 | 1.580663266 | 0.364231317 | 4.339723659 | 1.43E-05 | 0.000132496 | PLCB1 |
| 225.7439332 | -1.57905282 | 0.1844246 | -8.562050931 | 1.11E-17 | 4.27E-16 | HIST1H2AJ |
| 105.6392088 | -1.578599926 | 0.187485416 | -8.419854527 | 3.77E-17 | 1.40E-15 | KIF18A |
| 466.1571922 | -1.576418947 | 0.124249925 | -12.68748408 | 6.94E-37 | 8.24E-35 | FEN1 |
| 76.99680498 | -1.573570167 | 0.221439732 | -7.106087735 | 1.19E-12 | 2.99E-11 | SKA1 |
| 235.2083453 | 1.572598127 | 0.144798989 | 10.86056011 | 1.78E-27 | 1.24E-25 | KCNG1 |
| 26.58501856 | 1.572007526 | 0.319743761 | 4.916460356 | 8.81E-07 | 1.05E-05 | NBPF3 |
| 26.98983965 | 1.571721031 | 0.306159668 | 5.133664542 | 2.84E-07 | 3.67E-06 | IRF5 |
| 161.4557958 | -1.571305988 | 0.160541659 | -9.787528048 | 1.27E-22 | 6.86E-21 | CCNF |
| 96.639018 | -1.568092062 | 0.198313811 | -7.907124858 | 2.63E-15 | 8.35E-14 | RFC2 |
| 16.69877159 | 1.566796659 | 0.344608388 | 4.546600464 | 5.45E-06 | 5.57E-05 | HYAL1 |
| 10.29834603 | 1.566787084 | 0.364836545 | 4.294490515 | 1.75E-05 | 0.000159899 | CAND2 |
| 99.15547596 | -1.565380213 | 0.201172224 | -7.781293975 | 7.18E-15 | 2.17E-13 | ZNF724 |
| 240.3463396 | 1.565024208 | 0.146709993 | 10.66746838 | 1.45E-26 | 9.47E-25 | LCE1C |
| 263.7883169 | -1.56468189 | 0.135491149 | -11.54822212 | 7.54E-31 | 6.24E-29 | BUB1 |
| 19.24204777 | -1.56396976 | 0.335633671 | -4.659752274 | 3.17E-06 | 3.40E-05 | CSF1 |
| 116.6434989 | -1.562941135 | 0.190672633 | -8.196987216 | 2.46E-16 | 8.59E-15 | POLA2 |
| 24.16471394 | 1.561896556 | 0.314636891 | 4.964124036 | 6.90E-07 | 8.37E-06 | SAMD3 |
| 6.41124827 | 1.56151759 | 0.359395275 | 4.344847295 | 1.39E-05 | 0.00012977 | NA |
| 289.310227 | -1.561136709 | 0.135087731 | -11.55646553 | 6.85E-31 | 5.72E-29 | CCNB2 |
| 76.82938117 | -1.559271944 | 0.228388954 | -6.827265128 | 8.65E-12 | 1.99E-10 | MEF2C |
| 7.86677926 | 1.558361323 | 0.366613264 | 4.250695422 | 2.13E-05 | 0.000191622 | ROBO2 |
| 95.11859763 | -1.558000922 | 0.210355468 | -7.406514948 | 1.30E-13 | 3.55E-12 | RAD51 |
| 9.530776103 | -1.556478061 | 0.365705984 | -4.256091317 | 2.08E-05 | 0.000187572 | ZBTB7C |
| 419.1547228 | -1.551080765 | 0.124721636 | -12.43634077 | 1.66E-35 | 1.79E-33 | AURKB |
| 222.9897496 | 1.55077099 | 0.154207118 | 10.05641643 | 8.61E-24 | 4.91E-22 | CPA4 |
| 170.8839925 | 1.550750159 | 0.156646338 | 9.899689861 | 4.18E-23 | 2.30E-21 | GJB6 |
| 63.63540568 | -1.550571443 | 0.23314943 | -6.650547862 | 2.92E-11 | 6.20E-10 | CDC25C |
| 48.3406719 | 1.550137675 | 0.273215502 | 5.673681259 | 1.40E-08 | 2.19E-07 | CRIP2 |
| 8.551171635 | 1.54684843 | 0.366501236 | 4.220581758 | 2.44E-05 | 0.000215905 | COX7A1 |
| 8.980914336 | 1.545958243 | 0.366602812 | 4.216984139 | 2.48E-05 | 0.000219022 | TSLP |
| 231.6366404 | -1.543280537 | 0.14125429 | -10.92554808 | 8.70E-28 | 6.23E-26 | GPNMB |
| 13.23083348 | -1.54161959 | 0.355696521 | -4.334086777 | 1.46E-05 | 0.000135476 | NA |
| 1581.637424 | 1.541601904 | 0.098607826 | 15.6336669 | 4.29E-55 | 1.15E-52 | IL1B |
| 1107.745316 | 1.541448057 | 0.088205125 | 17.47571985 | 2.19E-68 | 9.46E-66 | MAP1B |
| 529.3965158 | -1.541117853 | 0.106622579 | -14.45395394 | 2.37E-47 | 4.51E-45 | SGO2 |
| 568.1159363 | -1.540574751 | 0.10327609 | -14.91705146 | 2.55E-50 | 5.50E-48 | HMMR |
| 85.3814292 | -1.538921258 | 0.209898941 | -7.331724731 | 2.27E-13 | 6.10E-12 | DDIAS |
| 66.74534185 | -1.538022605 | 0.22691894 | -6.777850287 | 1.22E-11 | 2.74E-10 | CENPA |
| 22.86123596 | -1.53138216 | 0.320670082 | -4.775569174 | 1.79E-06 | 2.01E-05 | ARHGAP24 |
| 34.1631309 | -1.527468466 | 0.288780515 | -5.289375106 | 1.23E-07 | 1.69E-06 | CXCL11 |
| 231.8736012 | -1.525718245 | 0.145005063 | -10.52182743 | 6.85E-26 | 4.33E-24 | ZWINT |
| 330.7528026 | -1.525250076 | 0.128755728 | -11.84607548 | 2.26E-32 | 2.01E-30 | TYMS |
| 898.5169208 | 1.524207251 | 0.231348795 | 6.588351803 | 4.45E-11 | 9.27E-10 | PI3 |
| 65.42208502 | -1.52329683 | 0.225958045 | -6.74150295 | 1.57E-11 | 3.44E-10 | POLE2 |
| 45.38560085 | 1.522942595 | 0.278795827 | 5.462573128 | 4.69E-08 | 6.87E-07 | COL6A1 |
| 118.7968703 | -1.521119133 | 0.187659138 | -8.105755734 | 5.24E-16 | 1.78E-14 | ACAA2 |
| 3526.061425 | 1.520087505 | 0.072389396 | 20.99875929 | 6.73E-98 | 7.88E-95 | PYGB |
| 89.41217093 | 1.512024776 | 0.207240531 | 7.295989675 | 2.96E-13 | 7.85E-12 | SLIT2 |
| 914.3814823 | 1.511006657 | 0.103463103 | 14.60430447 | 2.64E-48 | 5.20E-46 | SERPINB1 |
| 450.5008333 | -1.510546632 | 0.116388792 | -12.97845441 | 1.62E-38 | 2.06E-36 | ARHGAP11A |
| 214.4033311 | -1.509580905 | 0.142064704 | -10.62600955 | 2.26E-26 | 1.46E-24 | KIF11 |
| 231.8596215 | 1.508298567 | 0.154501415 | 9.762360871 | 1.63E-22 | 8.69E-21 | TNFRSF25 |
| 6.333988624 | -1.506248769 | 0.361128231 | -4.1709527 | 3.03E-05 | 0.000262794 | HSD17B3 |
| 18.44594581 | 1.505706907 | 0.355624926 | 4.233974611 | 2.30E-05 | 0.000204651 | LCE2C |
| 42.02570528 | -1.505015117 | 0.26804235 | -5.614840771 | 1.97E-08 | 3.00E-07 | C20orfl97 |
| 17.24900867 | 1.50311714 | 0.340828379 | 4.410187746 | 1.03E-05 | 9.90E-05 | TH |
| 18.48078592 | 1.502961172 | 0.335774563 | 4.476101937 | 7.60E-06 | 7.56E-05 | ADSSL1 |
| 5.781342427 | 1.502218067 | 0.357993365 | 4.196217619 | 2.71E-05 | 0.000237527 | NA |
| 9.393725569 | -1.500733479 | 0.366265808 | -4.097388967 | 4.18E-05 | 0.000352493 | CXCL10 |
| 326.7822287 | 1.499697964 | 0.134883853 | 11.11843954 | 1.02E-28 | 7.70E-27 | XAF1 |
| 38.41236831 | -1.499516483 | 0.277475951 | -5.404131335 | 6.51E-08 | 9.33E-07 | NA |
| 45.88169463 | 1.499073129 | 0.261556493 | 5.731355053 | 9.96E-09 | 1.60E-07 | ZNF704 |
| 275.6937066 | -1.498624263 | 0.131093296 | -11.43173836 | 2.90E-30 | 2.32E-28 | BUB1B |
| 11.68217886 | -1.498516611 | 0.360646902 | -4.155079673 | 3.25E-05 | 0.000279944 | CYP1A1 |
| 333.5330032 | -1.496239013 | 0.132105065 | -11.32612903 | 9.74E-30 | 7.56E-28 | NLRP2 |
| 134.2751816 | 1.496191906 | 0.174014121 | 8.598106281 | 8.10E-18 | 3.15E-16 | MMP2 |
| 7.301162211 | -1.49178312 | 0.365692319 | -4.079339501 | 4.52E-05 | 0.000377125 | MTCO3P12 |
| 1089.480616 | 1.489876422 | 0.100965391 | 14.7563082 | 2.80E-49 | 5.81E-47 | SERPINB2 |
| 11.96818952 | 1.489431411 | 0.362834422 | 4.104989276 | 4.04E-05 | 0.000342159 | FAM110B |
| 374.1417045 | -1.488823097 | 0.123559243 | -12.0494676 | 1.95E-33 | 1.88E-31 | CHAF1A |
| 175.162267 | -1.487825642 | 0.156980347 | -9.477782853 | 2.60E-21 | 1.28E-19 | TTK |
| 252.8019722 | -1.486727267 | 0.137090289 | -10.8448766 | 2.11E-27 | 1.46E-25 | KIF4A |
| 107.1972813 | 1.485402017 | 0.213615862 | 6.953612931 | 3.56E-12 | 8.55E-11 | KRT13 |
| 95.02252835 | -1.484130342 | 0.195157553 | -7.604780427 | 2.85E-14 | 8.26E-13 | CENPN |
| 188.7429338 | -1.483834812 | 0.171615588 | -8.6462706 | 5.32E-18 | 2.11E-16 | TRIP13 |
| 7.89671238 | -1.483417905 | 0.366384626 | -4.048799544 | 5.15E-05 | 0.000424252 | ETV7 |
| 19.77988829 | 1.483365616 | 0.331028335 | 4.481083513 | 7.43E-06 | 7.40E-05 | FAAH |
| 47.49108737 | -1.480032307 | 0.258874924 | -5.717171386 | 1.08E-08 | 1.72E-07 | MNS1 |
| 48.67852418 | -1.478793405 | 0.264111332 | -5.599128941 | 2.15E-08 | 3.27E-07 | DSCC1 |
| 25.58392481 | 1.478122468 | 0.31207214 | 4.736476861 | 2.17E-06 | 2.41E-05 | LRRC6 |
| 88.74630306 | -1.47622337 | 0.214532941 | -6.881103508 | 5.94E-12 | 1.39E-10 | POLQ |
| 147.2519163 | -1.476007563 | 0.168554286 | -8.75686758 | 2.01E-18 | 8.14E-17 | TCF19 |
| 6.409321726 | -1.475530808 | 0.36377243 | -4.056191966 | 4.99E-05 | 0.000412297 | PYHIN1 |
| 5.59930165 | 1.474264235 | 0.357125399 | 4.128141655 | 3.66E-05 | 0.000312213 | RIPPLY3 |
| 610.5057369 | -1.474156826 | 0.098781934 | -14.92334438 | 2.32E-50 | 5.08E-48 | GMNN |
| 75.09756961 | -1.471328707 | 0.217881199 | -6.75289431 | 1.45E-11 | 3.20E-10 | PLK4 |
| 5.439530898 | 1.469036869 | 0.357134414 | 4.113400478 | 3.90E-05 | 0.000331119 | PRMT8 |
| 138.1530225 | -1.467605628 | 0.174281691 | -8.420882443 | 3.74E-17 | 1.39E-15 | NEK2 |
| 72.27500757 | -1.467381313 | 0.226505359 | -6.478351409 | 9.27E-11 | 1.86E-09 | IL31RA |
| 41.28697957 | -1.467259466 | 0.273882637 | -5.357256242 | 8.45E-08 | 1.19E-06 | LMCD1 |
| 340.1349425 | -1.462447907 | 0.129978342 | -11.25147381 | 2.28E-29 | 1.76E-27 | FANCD2 |
| 670.342867 | -1.461408703 | 0.097595396 | -14.97415624 | 1.08E-50 | 2.43E-48 | CEP55 |
| 27.11600428 | -1.460412332 | 0.302794843 | -4.823108335 | 1.41E-06 | 1.62E-05 | KLRG2 |
| 47.18706769 | 1.460155638 | 0.273068949 | 5.347204953 | 8.93E-08 | 1.25E-06 | THNSL2 |
| 89.31333068 | 1.457570767 | 0.212819958 | 6.848844334 | 7.44E-12 | 1.72E-10 | EPB41L4A |
| 225.5473559 | 1.455496996 | 0.157900433 | 9.217815121 | 3.03E-20 | 1.40E-18 | MDK |
| 351.1623888 | -1.455057996 | 0.126007795 | -11.54736492 | 7.61E-31 | 6.27E-29 | H2AFX |
| 5.015941525 | -1.454307612 | 0.355196694 | -4.094372603 | 4.23E-05 | 0.000356111 | DAW1 |
| 123.8364364 | -1.451638675 | 0.179680618 | -8.078994218 | 6.53E-16 | 2.19E-14 | THEM6 |
| 626.9910581 | -1.450011425 | 0.100257151 | -14.46292273 | 2.08E-47 | 4.00E-45 | NUSAP1 |
| 348.1892284 | -1.449434495 | 0.119173887 | -12.1623498 | 4.93E-34 | 4.99E-32 | CDCA2 |
| 8.372665051 | -1.449298318 | 0.366619789 | -3.95313718 | 7.71E-05 | 0.000609881 | TRAC |
| 7.553340763 | 1.448468103 | 0.365972905 | 3.957856125 | 7.56E-05 | 0.000599406 | PRKN |
| 101.1762277 | 1.447462297 | 0.196853111 | 7.353006965 | 1.94E-13 | 5.25E-12 | LIMD2 |
| 5.767851591 | 1.447360402 | 0.361422288 | 4.004624091 | 6.21E-05 | 0.000501799 | SLC47A1 |
| 741.1408588 | -1.446770325 | 0.099618121 | -14.5231641 | 8.64E-48 | 1.69E-45 | CCNB1 |
| 4606.281859 | 1.445786539 | 0.06743782 | 21.43880898 | 5.81E-102 | 7.93E-99 | PTHLH |
| 8.37667671 | 1.443979567 | 0.36633361 | 3.941706487 | 8.09E-05 | 0.000636623 | PNMA6A |
| 771.2376706 | 1.44317752 | 0.100571275 | 14.34979834 | 1.07E-46 | 1.90E-44 | STC2 |
| 1770.648659 | 1.44289678 | 0.103123757 | 13.99189494 | 1.75E-44 | 2.95E-42 | ADAM8 |
| 9.403571983 | -1.438932632 | 0.365776298 | -3.933914361 | 8.36E-05 | 0.0006548 | CAMK4 |
| 116.0519579 | 1.432768859 | 0.211810925 | 6.764376559 | 1.34E-11 | 3.00E-10 | SULT2B1 |
| 56.03844001 | -1.432304845 | 0.250633084 | -5.714747718 | 1.10E-08 | 1.74E-07 | MT-TC |
| 31.47077057 | 1.429746545 | 0.302806415 | 4.721652094 | 2.34E-06 | 2.58E-05 | AMT |
| 381.1758696 | 1.429659535 | 0.134534239 | 10.62673372 | 2.24E-26 | 1.46E-24 | MIR503HG |
| 844.3806322 | -1.42946143 | 0.099535035 | -14.3613898 | 9.04E-47 | 1.63E-44 | CBR1 |
| 494.0058573 | 1.425413595 | 0.119287996 | 11.9493465 | 6.54E-33 | 6.13E-31 | RFLNB |
| 355.670225 | -1.423352869 | 0.119332133 | -11.92765797 | 8.49E-33 | 7.77E-31 | MCM5 |
| 278.9527987 | 1.422915152 | 0.130320667 | 10.9185687 | 9.40E-28 | 6.69E-26 | ADGRL1 |
| 43.79601493 | 1.421718876 | 0.263178913 | 5.40210027 | 6.59E-08 | 9.42E-07 | BAMBI |
| 6.301981137 | 1.421335918 | 0.364186744 | 3.90276676 | 9.51E-05 | 0.000736649 | ARHGEF25 |
| 9.091511448 | 1.420581013 | 0.366294002 | 3.878253552 | 0.000105209 | 0.00080132 | IFNE |
| 63.62115808 | 1.417161454 | 0.229800705 | 6.166915172 | 6.96E-10 | 1.27E-08 | DBNDD1 |
| 12.40209918 | 1.41581324 | 0.359638603 | 3.936766595 | 8.26E-05 | 0.00064862 | SLCO2A1 |
| 127.0800844 | -1.415174462 | 0.181058878 | -7.816100896 | 5.45E-15 | 1.67E-13 | FHOD3 |
| 53.1925153 | -1.408883099 | 0.240637879 | -5.854785238 | 4.78E-09 | 7.97E-08 | SPESP1 |
| 126.9526283 | -1.407705634 | 0.179260023 | -7.852869868 | 4.07E-15 | 1.27E-13 | CIT |
| 121.4335406 | -1.406461138 | 0.18249196 | -7.706975901 | 1.29E-14 | 3.83E-13 | BLM |
| 886.4941826 | -1.401308072 | 0.095213123 | -14.71759388 | 4.97E-49 | 1.02E-46 | PRC1 |
| 490.727929 | -1.400858095 | 0.123309754 | -11.36048085 | 6.58E-30 | 5.18E-28 | PEG10 |
| 113.5334938 | -1.400517023 | 0.188014791 | -7.448972574 | 9.41E-14 | 2.61E-12 | MND1 |
| 220.1598434 | -1.400118908 | 0.145933228 | -9.594243395 | 8.45E-22 | 4.29E-20 | NCAPG2 |
| 203.6926962 | 1.399954518 | 0.166143793 | 8.426162 | 3.57E-17 | 1.33E-15 | FKBP10 |
| 152.5899766 | -1.396476732 | 0.172408432 | -8.099816901 | 5.50E-16 | 1.86E-14 | SKA3 |
| 41.73345692 | -1.39619567 | 0.269947113 | -5.172108176 | 2.31E-07 | 3.03E-06 | APLN |
| 47.1894848 | 1.39304744 | 0.262116097 | 5.314619965 | 1.07E-07 | 1.48E-06 | ZFHX2 |
| 5.236010531 | -1.392429067 | 0.358767803 | -3.88114278 | 0.000103967 | 0.000793704 | SNORA23 |
| 64.65142639 | -1.389772501 | 0.231357645 | -6.007030804 | 1.89E-09 | 3.29E-08 | CENPP |
| 30.10037663 | 1.389506974 | 0.300243927 | 4.627926993 | 3.69E-06 | 3.90E-05 | ECM2 |
| 11.67182176 | -1.388232587 | 0.358282094 | -3.874691508 | 0.00010676 | 0.000812377 | ZNF726 |
| 213.6029067 | -1.38760455 | 0.154404336 | -8.986823739 | 2.54E-19 | 1.07E-17 | CENPK |
| 8.282231694 | -1.386636275 | 0.366574188 | -3.78268934 | 0.000155143 | 0.001133679 | GAD1 |
| 42.4594558 | -1.385508977 | 0.274187842 | -5.053137911 | 4.35E-07 | 5.44E-06 | MMP17 |
| 639.702499 | -1.383597116 | 0.10407884 | -13.29374072 | 2.52E-40 | 3.41E-38 | IFIT1 |
| 10.53920144 | -1.382850104 | 0.365260936 | -3.785923888 | 0.000153139 | 0.001120531 | CD70 |
| 41.13261441 | -1.382445162 | 0.273441675 | -5.055722249 | 4.29E-07 | 5.38E-06 | NA |
| 346.2386994 | -1.381265194 | 0.136799445 | -10.09700876 | 5.70E-24 | 3.29E-22 | MCM2 |
| 6.352708376 | -1.378855326 | 0.364662617 | -3.78118091 | 0.000156086 | 0.001139554 | NA |
| 34.69377337 | 1.377414802 | 0.28356589 | 4.857477047 | 1.19E-06 | 1.39E-05 | CREB3L1 |
| 648.3954433 | 1.376631872 | 0.10073287 | 13.6661635 | 1.62E-42 | 2.39E-40 | PCK2 |
| 17.95352843 | 1.376422653 | 0.347043604 | 3.966137502 | 7.30E-05 | 0.000582631 | LCE2B |
| 13.26007088 | -1.375178693 | 0.357008097 | -3.851953788 | 0.000117179 | 0.000881426 | HRCT1 |
| 28.79990637 | 1.374747017 | 0.303879003 | 4.523994751 | 6.07E-06 | 6.16E-05 | MEST |
| 12.94786953 | -1.373873069 | 0.360925775 | -3.806525225 | 0.000140933 | 0.001039579 | NA |
| 13.43994975 | 1.37367671 | 0.356095924 | 3.857603013 | 0.000114504 | 0.000864084 | EFCAB13 |
| 446.8418452 | -1.373357871 | 0.117744123 | -11.66391861 | 1.95E-31 | 1.66E-29 | VCAN |
| 82.93939185 | -1.373203324 | 0.214335038 | -6.406807488 | 1.49E-10 | 2.91E-09 | APOBEC3G |
| 25.97550777 | 1.37280693 | 0.309990814 | 4.428540673 | 9.49E-06 | 9.18E-05 | TMEM132B |
| 53.35795072 | 1.372357116 | 0.289970475 | 4.732747762 | 2.22E-06 | 2.45E-05 | KPRP |
| 92.72746768 | -1.371967369 | 0.198426036 | -6.914250745 | 4.70E-12 | 1.11E-10 | IQGAP3 |
| 4.58182126 | -1.370121141 | 0.351933659 | -3.893123338 | 9.90E-05 | 0.000760456 | UPK3BL1 |
| 89.17385734 | -1.368522921 | 0.222553453 | -6.149187548 | 7.79E-10 | 1.41E-08 | XRCC2 |
| 4.848761727 | 1.368443239 | 0.353557134 | 3.870500989 | 0.000108612 | 0.00082341 | ZNF208 |
| 105.0209506 | -1.367555513 | 0.197654031 | -6.918935614 | 4.55E-12 | 1.08E-10 | GAMT |
| 13.30255434 | -1.366212206 | 0.353357332 | -3.86637571 | 0.000110465 | 0.000835523 | HSH2D |
| 10.65919471 | 1.365337971 | 0.362202646 | 3.769541681 | 0.000163548 | 0.001188204 | KIF21B |
| 7.982701693 | 1.365300786 | 0.366652394 | 3.723692543 | 0.00019633 | 0.001403961 | FIBIN |
| 87.27825577 | -1.364945829 | 0.208113997 | -6.558645009 | 5.43E-11 | 1.12E-09 | DDAH1 |
| 60.96127134 | -1.363302515 | 0.231947258 | -5.877640152 | 4.16E-09 | 6.99E-08 | ZGRF1 |
| 96.4956854 | -1.359664847 | 0.196203521 | -6.929869748 | 4.21E-12 | 1.01E-10 | MMS22L |
| 51.62178422 | -1.358611314 | 0.24844927 | -5.468365091 | 4.54E-08 | 6.66E-07 | FANCB |
| 27.86646187 | -1.357211989 | 0.303595034 | -4.470468336 | 7.80E-06 | 7.74E-05 | NA |
| 180.52331 | -1.356656527 | 0.157655567 | -8.605192661 | 7.62E-18 | 2.98E-16 | MASTL |
| 28.60148125 | -1.356298265 | 0.304465554 | -4.454685422 | 8.40E-06 | 8.24E-05 | RAD54L |
| 270.7611834 | 1.355556443 | 0.143650867 | 9.436465442 | 3.86E-21 | 1.89E-19 | CLDN4 |
| 8.007112146 | -1.354973644 | 0.366601131 | -3.696043273 | 0.000218986 | 0.001542408 | CDKN2B-AS1 |
| 161.30197 | -1.354162554 | 0.168097155 | -8.055832661 | 7.89E-16 | 2.63E-14 | CDKN3 |
| 170.5099024 | -1.353894725 | 0.156225403 | -8.666290517 | 4.46E-18 | 1.78E-16 | PRIM1 |
| 16.07875322 | 1.351557239 | 0.344971171 | 3.917884602 | 8.93E-05 | 0.000696566 | TNRC6C-AS1 |
| 215.1200834 | -1.347050389 | 0.148958252 | -9.043140401 | 1.52E-19 | 6.60E-18 | TK1 |
| 8.424242319 | -1.346451687 | 0.366052144 | -3.678305697 | 0.000234788 | 0.001632657 | MTCO3P22 |
| 35.00676121 | 1.345233436 | 0.288054669 | 4.670062938 | 3.01E-06 | 3.25E-05 | KYNU |
| 9.611075011 | 1.34419572 | 0.364450626 | 3.688279354 | 0.000225776 | 0.001578695 | TRO |
| 47.03617619 | -1.34253612 | 0.253563243 | -5.294679561 | 1.19E-07 | 1.64E-06 | BTN3A2 |
| 130.3853575 | 1.342160798 | 0.186236681 | 7.206747823 | 5.73E-13 | 1.48E-11 | GOLGA8A |
| 9.300714848 | 1.342160413 | 0.366512149 | 3.661980688 | 0.000250273 | 0.001726408 | MAPT |
| 108.2540101 | 1.342086071 | 0.187990648 | 7.139110829 | 9.39E-13 | 2.38E-11 | HSPA2 |
| 874.3850791 | -1.341886597 | 0.088425499 | -15.17533534 | 5.15E-52 | 1.21E-49 | GPSM2 |
| 1310.716084 | 1.341621945 | 0.086376262 | 15.53229926 | 2.10E-54 | 5.29E-52 | ASNS |
| 94.93004281 | -1.340369718 | 0.203491912 | -6.586845172 | 4.49E-11 | 9.35E-10 | SELENOM |
| 350.8814774 | -1.339695126 | 0.125745229 | -10.65404337 | 1.67E-26 | 1.09E-24 | KNL1 |
| 261.2208985 | -1.338043011 | 0.134492342 | -9.948841667 | 2.55E-23 | 1.41E-21 | NUF2 |
| 7.315904614 | 1.337197067 | 0.366348075 | 3.650072594 | 0.000262166 | 0.001800867 | EDA |
| 45.29893007 | 1.336236928 | 0.263143165 | 5.077984558 | 3.81E-07 | 4.82E-06 | TRPV3 |
| 213.0097117 | -1.335871158 | 0.150801564 | -8.858470167 | 8.11E-19 | 3.34E-17 | CENPU |
| 41.52328023 | 1.335863294 | 0.273496282 | 4.884392886 | 1.04E-06 | 1.22E-05 | SERPINI1 |
| 84.58809498 | -1.335848628 | 0.228085409 | -5.856791248 | 4.72E-09 | 7.88E-08 | OIP5 |
| 2450.868771 | -1.335781682 | 0.073258995 | -18.23368835 | 2.79E-74 | 1.58E-71 | MDM2 |
| 40.72423106 | 1.335274598 | 0.272304911 | 4.90360087 | 9.41E-07 | 1.11E-05 | AZIN2 |
| 10.10962507 | -1.334719218 | 0.362652824 | -3.680432441 | 0.000232839 | 0.001621853 | UBE25 |
| 10073.25016 | 1.334652529 | 0.079494396 | 16.78926554 | 2.92E-63 | 1.09E-60 | CSTA |
| 265.9798785 | -1.334462778 | 0.152276263 | -8.763432683 | 1.89E-18 | 7.70E-17 | HIST1H1D |
| 313.0053761 | -1.334339233 | 0.133742949 | -9.976894054 | 1.92E-23 | 1.07E-21 | CENPW |
| 224.3116629 | -1.334145321 | 0.14947221 | -8.925708135 | 4.43E-19 | 1.84E-17 | NEFM |
| 13.60091323 | -1.333693775 | 0.35208505 | -3.787987522 | 0.000151872 | 0.001111764 | LINC00911 |
| 14.50210611 | 1.333089573 | 0.35444224 | 3.761091153 | 0.000169174 | 0.001225818 | RAET1L |
| 81.07543899 | -1.332331084 | 0.207942828 | -6.407199025 | 1.48E-10 | 2.90E-09 | GAS2L3 |
| 19.65834162 | -1.331762779 | 0.329158128 | -4.045966563 | 5.21E-05 | 0.000428769 | GPR3 |
| 146.3589318 | -1.330627736 | 0.166941078 | -7.970643008 | 1.58E-15 | 5.15E-14 | WDR76 |
| 169.0052119 | 1.329203629 | 0.159120048 | 8.35346421 | 6.63E-17 | 2.41E-15 | SH2D2A |
| 40.41865787 | -1.328130984 | 0.272736468 | -4.869649415 | 1.12E-06 | 1.31E-05 | SFXN2 |
| 4.930081662 | -1.328094921 | 0.35681196 | -3.72211436 | 0.000197562 | 0.001411535 | MTND3P10 |
| 557.8152566 | -1.327320483 | 0.106651186 | -12.44543577 | 1.48E-35 | 1.61E-33 | NCAPD2 |
| 254.9088894 | -1.326352834 | 0.161060177 | -8.235138298 | 1.79E-16 | 6.31E-15 | PSMB9 |
| 9.448461056 | -1.326102308 | 0.364281503 | -3.640322926 | 0.000272296 | 0.0018642 | NA |
| 34.67283174 | 1.323608038 | 0.292506054 | 4.525062027 | 6.04E-06 | 6.13E-05 | SH3D21 |
| 1526.339126 | 1.323597459 | 0.086382315 | 15.32255129 | 5.41E-53 | 1.28E-50 | PMEPA1 |
| 72.39654593 | 1.323476535 | 0.218634525 | 6.053373931 | 1.42E-09 | 2.49E-08 | GSTM4 |
| 41.65868524 | 1.322138026 | 0.286060794 | 4.621877782 | 3.80E-06 | 4.00E-05 | NOTCH3 |
| 127.8734403 | 1.321841562 | 0.200785728 | 6.583344232 | 4.60E-11 | 9.55E-10 | AHSA2P |
| 332.0592503 | -1.32142553 | 0.120280149 | -10.98623121 | 4.45E-28 | 3.23E-26 | CDCA7 |
| 13.87354994 | 1.321166321 | 0.34976041 | 3.777346675 | 0.000158508 | 0.001155213 | TSPAN33 |
| 11.91537354 | 1.320473803 | 0.356773038 | 3.701159177 | 0.000214617 | 0.001516854 | COX6B2 |
| 8.248322737 | -1.318981983 | 0.366305665 | -3.600768723 | 0.000317278 | 0.00213381 | APOL3 |
| 56.79800705 | 1.318606519 | 0.239100888 | 5.514854131 | 3.49E-08 | 5.18E-07 | ABCA7 |
| 6.974413475 | 1.318604513 | 0.366318826 | 3.599608916 | 0.000318696 | 0.002141591 | PAMR1 |
| 16.54672328 | 1.316835335 | 0.345619832 | 3.810068787 | 0.000138928 | 0.001027103 | BEGAIN |
| 39.83049294 | -1.316451277 | 0.285760941 | -4.606827203 | 4.09E-06 | 4.27E-05 | FBXO32 |
| 1858.38226 | -1.315362187 | 0.073849916 | -17.81128878 | 5.78E-71 | 2.87E-68 | SP110 |
| 12.02715194 | 1.314137069 | 0.359530001 | 3.655152739 | 0.000257029 | 0.001769288 | TLL1 |
| 295.2164035 | -1.313917731 | 0.125753675 | -10.44834461 | 1.49E-25 | 9.22E-24 | KIF14 |
| 1133.519777 | -1.312901791 | 0.084413296 | -15.55325832 | 1.51E-54 | 3.87E-52 | TPX2 |
| 137.4384981 | -1.310723547 | 0.198331774 | -6.608742099 | 3.88E-11 | 8.15E-10 | MT-TP |
| 56.54688129 | 1.308465296 | 0.241391378 | 5.420513803 | 5.94E-08 | 8.54E-07 | OPLAH |
| 8.471890117 | 1.308464019 | 0.366650865 | 3.568692027 | 0.000358768 | 0.002372908 | CILP2 |
| 4.561433087 | 1.308265855 | 0.350952285 | 3.727759899 | 0.000193189 | 0.001383918 | NA |
| 56.11349707 | 1.307528948 | 0.257128318 | 5.085122319 | 3.67E-07 | 4.65E-06 | FUT3 |
| 459.1625598 | 1.303468793 | 0.122015022 | 10.68285504 | 1.22E-26 | 8.12E-25 | TAGLN |
| 5.678667797 | 1.303370295 | 0.36229126 | 3.597575869 | 0.000321197 | 0.002156626 | PCSK4 |
| 18.04658791 | 1.30255893 | 0.334598687 | 3.892899114 | 9.91E-05 | 0.000760802 | SYCP2L |
| 318.8189538 | 1.302325766 | 0.124363993 | 10.47188769 | 1.16E-25 | 7.24E-24 | RPS6KA2 |
| 11.7184985 | 1.302126764 | 0.363351256 | 3.583658358 | 0.000338815 | 0.002256427 | RASL10B |
| 6.514021391 | -1.301656636 | 0.365352886 | -3.562738067 | 0.000367007 | 0.002420561 | RTP4 |
| 17.96676913 | 1.301182876 | 0.33265502 | 3.911508312 | 9.17E-05 | 0.000713181 | ZNF322 |
| 48.27456923 | -1.299889113 | 0.253850766 | -5.120682249 | 3.04E-07 | 3.92E-06 | FAM171B |
| 768.3151969 | -1.298822637 | 0.102444941 | -12.67825068 | 7.81E-37 | 9.13E-35 | COL8A1 |
| 8.192781018 | 1.2986897 | 0.366463501 | 3.543844604 | 0.000394338 | 0.002571828 | SMOC2 |
| 593.66388 | -1.29864349 | 0.1009593 | -12.86303972 | 7.27E-38 | 8.82E-36 | MCM3 |
| 8300.758341 | 1.297931641 | 0.07890055 | 16.45022289 | 8.36E-61 | 2.79E-58 | FERMT1 |
| 5.996255051 | 1.29505432 | 0.360298764 | 3.594390125 | 0.000325152 | 0.002177829 | NA |
| 709.1605242 | -1.294193763 | 0.092024633 | -14.06355805 | 6.36E-45 | 1.09E-42 | AURKA |
| 170.7870677 | 1.293584872 | 0.163503417 | 7.911668707 | 2.54E-15 | 8.06E-14 | MYL9 |
| 14.18532493 | 1.292979668 | 0.349860491 | 3.695700723 | 0.000219281 | 0.001543412 | ARHGAP45 |
| 891.9500306 | 1.292757804 | 0.093437774 | 13.8354945 | 1.56E-43 | 2.45E-41 | IFNK |
| 15.32843997 | 1.289149053 | 0.350747176 | 3.675436731 | 0.000237443 | 0.001646287 | RBP7 |
| 48.82008448 | 1.287408975 | 0.25056742 | 5.137974337 | 2.78E-07 | 3.60E-06 | CCDC149 |
| 81.18593756 | -1.287153036 | 0.207082981 | -6.215638905 | 5.11E-10 | 9.43E-09 | CENPO |
| 77.84330321 | -1.286581056 | 0.211604116 | -6.080132466 | 1.20E-09 | 2.13E-08 | USP18 |
| 227.2272738 | -1.286386736 | 0.152605443 | -8.429494467 | 3.47E-17 | 1.30E-15 | CENPH |
| 17.39658191 | -1.286335418 | 0.336275251 | -3.825245584 | 0.000130642 | 0.000971541 | PLIN4 |
| 14.11454955 | -1.284398185 | 0.34945551 | -3.675426907 | 0.000237452 | 0.001646287 | NA |
| 6.384281158 | 1.282308307 | 0.36359106 | 3.52678723 | 0.000420635 | 0.002722741 | DUSP9 |
| 4.396280788 | 1.281409466 | 0.349757383 | 3.663709552 | 0.000248589 | 0.001716236 | CFAP46 |
| 22.00772859 | -1.279915488 | 0.325101858 | -3.936967614 | 8.25E-05 | 0.000648388 | SLITRK5 |
| 4.176012181 | -1.279813834 | 0.347636783 | -3.681468407 | 0.000231895 | 0.00161665 | U2AF1 |
| 32.48928402 | 1.279516394 | 0.298445592 | 4.287268526 | 1.81E-05 | 0.000165001 | LEMD1 |
| 765.6833183 | -1.278141518 | 0.111218715 | -11.49214427 | 1.44E-30 | 1.17E-28 | MCM4 |
| 692.3908368 | -1.277546222 | 0.100167581 | -12.75408875 | 2.96E-37 | 3.54E-35 | IFIT3 |
| 6.547806354 | -1.277410671 | 0.364769221 | -3.50196946 | 0.000461833 | 0.002959016 | RIBC2 |
| 74.90416104 | -1.277200563 | 0.211857315 | -6.028588451 | 1.65E-09 | 2.89E-08 | FANCA |
| 5.531935325 | 1.27680239 | 0.36171747 | 3.529833355 | 0.000415821 | 0.002697982 | BGN |
| 1232.72572 | -1.27631125 | 0.101155077 | -12.6173721 | 1.69E-36 | 1.95E-34 | KLK7 |
| 7.103182321 | 1.273527241 | 0.36567281 | 3.482696024 | 0.000496392 | 0.003148426 | NACA3P |
| 302.0297975 | 1.273211585 | 0.130607557 | 9.748376056 | 1.87E-22 | 9.94E-21 | RSRP1 |
| 13.41035012 | 1.271662407 | 0.351813097 | 3.614596549 | 0.000300816 | 0.002032274 | DAPK2 |
| 32.18016403 | -1.271157346 | 0.285712433 | -4.449079562 | 8.62E-06 | 8.42E-05 | NA |
| 401.2878388 | -1.270431908 | 0.116694052 | -10.88686086 | 1.33E-27 | 9.40E-26 | FBN2 |
| 64.49345913 | 1.269494545 | 0.234795118 | 5.406818312 | 6.42E-08 | 9.20E-07 | PABPC1L |
| 8.181153968 | -1.267708504 | 0.366633556 | -3.457699063 | 0.00054481 | 0.00341585 | BCL2A1 |
| 358.4674268 | -1.266977427 | 0.129166364 | -9.808880521 | 1.03E-22 | 5.59E-21 | RFC3 |
| 541.0687793 | -1.266188151 | 0.105081823 | -12.04954492 | 1.95E-33 | 1.88E-31 | VRK1 |
| 29.53513905 | -1.26504827 | 0.303568092 | -4.167263638 | 3.08E-05 | 0.0002668 | RAB17 |
| 62.99129717 | -1.264883132 | 0.232555654 | -5.439055593 | 5.36E-08 | 7.76E-07 | BAG2 |
| 86.75083905 | 1.264535619 | 0.20239366 | 6.24790134 | 4.16E-10 | 7.77E-09 | PTPRM |
| 77.47835607 | -1.263052793 | 0.225836387 | -5.59277808 | 2.23E-08 | 3.39E-07 | SLC25A19 |
| 22.93583517 | -1.262730545 | 0.313217255 | -4.031484613 | 5.54E-05 | 0.000453538 | NA |
| 11.18053625 | -1.261470865 | 0.359654749 | -3.507449486 | 0.000452424 | 0.002903294 | FAM49A |
| 8.64519229 | 1.260529785 | 0.366510802 | 3.439270487 | 0.000583284 | 0.00362242 | NA |
| 120.5250094 | -1.259407594 | 0.192689921 | -6.535928736 | 6.32E-11 | 1.29E-09 | SIPA1L3 |
| 118.986988 | 1.257652533 | 0.186038287 | 6.760181227 | 1.38E-11 | 3.07E-10 | MYEF2 |
| 13.48504835 | 1.256868679 | 0.352426972 | 3.566323745 | 0.000362024 | 0.002391549 | RNF208 |
| 3650.461673 | 1.256617448 | 0.087250823 | 14.4023564 | 5.00E-47 | 9.10E-45 | KRT16 |
| 8.530519353 | -1.256492175 | 0.366186134 | -3.431293703 | 0.00060071 | 0.003710946 | DPYD |
| 37.42764575 | -1.256101575 | 0.286767004 | -4.380216542 | 1.19E-05 | 0.000112083 | EME1 |
| 80.94848389 | -1.254874343 | 0.212619702 | -5.901966422 | 3.59E-09 | 6.08E-08 | PAX8 |
| 13.76568932 | -1.254639715 | 0.359889184 | -3.486183439 | 0.000489965 | 0.003117318 | NA |
| 13.45236837 | 1.253865637 | 0.35321573 | 3.549857866 | 0.000385439 | 0.002518807 | TMEM121 |
| 35.31783269 | -1.253572385 | 0.300362476 | -4.173531933 | 3.00E-05 | 0.000260248 | SLC16A7 |
| 813.6016737 | -1.253289048 | 0.102331744 | -12.24731448 | 1.74E-34 | 1.79E-32 | ASPM |
| 414.7921774 | 1.252877064 | 0.115876196 | 10.81220393 | 3.01E-27 | 2.08E-25 | MTSS1 |
| 156.8439865 | 1.252066542 | 0.165807735 | 7.551315643 | 4.31E-14 | 1.24E-12 | LENG8 |
| 15.45767903 | -1.252048765 | 0.344497849 | -3.63441678 | 0.00027861 | 0.001902655 | ZNF488 |
| 12.6134251 | -1.251999134 | 0.35961076 | -3.481539689 | 0.00049854 | 0.003159482 | RPTN |
| 6.33704959 | -1.251499777 | 0.364607171 | -3.43246068 | 0.000598131 | 0.003697801 | KIAA1324 |
| 147.9801395 | -1.251404158 | 0.169920532 | -7.364643616 | 1.78E-13 | 4.82E-12 | RBL1 |
| 165.7917753 | 1.249483618 | 0.179470137 | 6.962069771 | 3.35E-12 | 8.07E-11 | ARRDC3 |
| 84.8885445 | -1.249348439 | 0.20567529 | -6.07437304 | 1.24E-09 | 2.21E-08 | PHEX |
| 422.4860263 | -1.247057724 | 0.117832931 | -10.58326999 | 3.56E-26 | 2.30E-24 | ANO4 |
| 53.8946185 | 1.246325814 | 0.248452591 | 5.016352661 | 5.27E-07 | 6.51E-06 | GPRC5C |
| 72.40453006 | -1.246264941 | 0.220164503 | -5.660607973 | 1.51E-08 | 2.35E-07 | LETM2 |
| 42.72755021 | 1.245961808 | 0.266839149 | 4.669336608 | 3.02E-06 | 3.26E-05 | SPOCK1 |
| 38.39605585 | 1.244396872 | 0.285883843 | 4.352805871 | 1.34E-05 | 0.000125755 | S1PR1 |
| 266.7174704 | -1.243762983 | 0.132848699 | -9.362251906 | 7.81E-21 | 3.75E-19 | GPX3 |
| 29.71312932 | 1.243580472 | 0.295428775 | 4.209408752 | 2.56E-05 | 0.0002254 | NA |
| 50.58241556 | -1.241961643 | 0.267734945 | -4.638773035 | 3.50E-06 | 3.72E-05 | PIR |
| 25.07577179 | 1.241867833 | 0.318659701 | 3.897159979 | 9.73E-05 | 0.000750359 | NA |
| 9.633488499 | 1.238723089 | 0.365125711 | 3.392593428 | 0.000692343 | 0.004196322 | LCE2D |
| 4.238041384 | 1.237293113 | 0.347429912 | 3.561274003 | 0.00036906 | 0.002431164 | ALDH1A1 |
| 46.83579134 | -1.236877692 | 0.257692191 | -4.799826063 | 1.59E-06 | 1.80E-05 | NKX3-1 |
| 70.78042 | 1.236678647 | 0.234763765 | 5.267757773 | 1.38E-07 | 1.88E-06 | CEP112 |
| 75.75058485 | 1.236506332 | 0.226417767 | 5.461171832 | 4.73E-08 | 6.91E-07 | PLXDC2 |
| 99.39007955 | 1.236460279 | 0.195709384 | 6.317838489 | 2.65E-10 | 5.07E-09 | C4orf33 |
| 17.87652601 | 1.236402873 | 0.340284642 | 3.633437183 | 0.000279671 | 0.001906816 | DMTN |
| 22.54575737 | 1.235963392 | 0.324125484 | 3.813224981 | 0.000137165 | 0.001015444 | SLC29A4 |
| 6.188539223 | 1.235220256 | 0.363347811 | 3.399553314 | 0.00067496 | 0.004106154 | NA |
| 11.0923246 | 1.233802634 | 0.361288456 | 3.415007074 | 0.000637804 | 0.003901843 | PODXL2 |
| 30.6474591 | -1.233618802 | 0.295133717 | -4.179864006 | 2.92E-05 | 0.000253644 | CNTNAP2 |
| 277.2453125 | -1.232990496 | 0.127935507 | -9.637594161 | 5.55E-22 | 2.87E-20 | CKS2 |
| 145.89235 | -1.232674963 | 0.163401518 | -7.543840357 | 4.56E-14 | 1.31E-12 | STIL |
| 11.9751199 | 1.231748547 | 0.359680699 | 3.424561144 | 0.000615793 | 0.00378699 | CDHR1 |
| 42.12421175 | -1.230281612 | 0.288175163 | -4.269214593 | 1.96E-05 | 0.000177456 | FAM13C |
| 46.93541392 | -1.230106017 | 0.257100689 | -4.784530232 | 1.71E-06 | 1.93E-05 | TNS3 |
| 464.8328179 | 1.229961638 | 0.110082947 | 11.17304426 | 5.53E-29 | 4.23E-27 | SYNPO |
| 278.2517974 | -1.229541056 | 0.134926076 | -9.112701502 | 8.04E-20 | 3.54E-18 | FANCI |
| 25.06289793 | -1.228219027 | 0.314200455 | -3.909030075 | 9.27E-05 | 0.000719171 | NA |
| 219.7165483 | -1.226317702 | 0.142799325 | -8.587699583 | 8.87E-18 | 3.44E-16 | ID3 |
| 19.29208535 | 1.22568211 | 0.329789765 | 3.716555946 | 0.000201957 | 0.001438548 | CCDC7 |
| 2260.392877 | -1.224394813 | 0.075848785 | -16.14257644 | 1.28E-58 | 3.89E-56 | CYGB |
| 144.2947064 | -1.224092915 | 0.169906177 | -7.204522717 | 5.82E-13 | 1.50E-11 | CDC25A |
| 95.13107217 | -1.22398983 | 0.203390061 | -6.017943173 | 1.77E-09 | 3.08E-08 | SYT8 |
| 6.962476366 | 1.223669146 | 0.366374845 | 3.339937672 | 0.000837972 | 0.004965097 | GCK |
| 33.6482199 | -1.222546753 | 0.293698346 | -4.162593254 | 3.15E-05 | 0.000271887 | C17orf53 |
| 4.209329479 | -1.22156721 | 0.343961034 | -3.551469752 | 0.000383086 | 0.002509436 | MINOS1P3 |
| 15.83231345 | -1.220732754 | 0.34507675 | -3.537568822 | 0.000403829 | 0.002625369 | HS3ST3A1 |
| 502.6633443 | 1.219544808 | 0.11099421 | 10.98746329 | 4.39E-28 | 3.20E-26 | MAP2 |
| 392.866991 | -1.219328611 | 0.112995789 | -10.79092078 | 3.80E-27 | 2.62E-25 | FAM111A |
| 16.72614712 | 1.218726935 | 0.350063376 | 3.481446552 | 0.000498713 | 0.003159482 | LCE2A |
| 842.5732879 | 1.218338035 | 0.091239219 | 13.3532274 | 1.13E-40 | 1.60E-38 | LINC01133 |
| 1383.070213 | -1.218036598 | 0.084480202 | -14.41801246 | 3.99E-47 | 7.42E-45 | KIF20B |
| 13.74529717 | 1.216312571 | 0.354319159 | 3.432816267 | 0.000597347 | 0.003694349 | LINC02057 |
| 38.02129609 | -1.214873291 | 0.271974603 | -4.466863008 | 7.94E-06 | 7.86E-05 | DOCK10 |
| 7.509573447 | -1.213179569 | 0.366611035 | -3.309173633 | 0.000935718 | 0.00549064 | GBGT1 |
| 31.74289489 | -1.212279425 | 0.296515554 | -4.088417659 | 4.34E-05 | 0.000364526 | TRANK1 |
| 149.9473451 | -1.210286654 | 0.180650459 | -6.699604663 | 2.09E-11 | 4.52E-10 | BRIP1 |
| 40.96766613 | 1.20941171 | 0.285533304 | 4.235623983 | 2.28E-05 | 0.000203265 | PTP4A3 |
| 761.92835 | 1.207022002 | 0.103664814 | 11.64350713 | 2.48E-31 | 2.10E-29 | PTPRZ1 |
| 4.092074585 | 1.206994895 | 0.345814392 | 3.490296886 | 0.000482484 | 0.0030745 | HNMT |
| 94.79206256 | -1.206435257 | 0.207281055 | -5.82028714 | 5.87E-09 | 9.71E-08 | PSMC3IP |
| 182.6405185 | -1.205764823 | 0.160692865 | -7.503536797 | 6.21E-14 | 1.75E-12 | TTC12 |
| 599.3295523 | -1.203252294 | 0.106219139 | -11.32801774 | 9.53E-30 | 7.44E-28 | CCBE1 |
| 8.751389078 | -1.202585212 | 0.366065982 | -3.2851597 | 0.001019246 | 0.005927663 | HIST1H2AG |
| 48.99125051 | -1.201483353 | 0.246910973 | -4.866058962 | 1.14E-06 | 1.33E-05 | PLB1 |
| 32.83636234 | 1.20001158 | 0.296224337 | 4.05102293 | 5.10E-05 | 0.000421088 | S100P |
| 176.5737215 | -1.199342171 | 0.152998694 | -7.838904643 | 4.54E-15 | 1.41E-13 | NCAPD3 |
| 8349.085459 | 1.196174911 | 0.177051753 | 6.756074933 | 1.42E-11 | 3.15E-10 | GOS2 |
| 9170.215765 | 1.195849386 | 0.082877259 | 14.42916189 | 3.39E-47 | 6.39E-45 | KRT6B |
| 246.3320785 | 1.195051045 | 0.137500344 | 8.691258585 | 3.58E-18 | 1.43E-16 | FAM171A1 |
| 184.7492774 | 1.194577068 | 0.155258568 | 7.694113669 | 1.42E-14 | 4.21E-13 | H6PD |
| 33.70339151 | 1.194330707 | 0.300691662 | 3.971944885 | 7.13E-05 | 0.000569715 | LYPD5 |
| 252.0928238 | -1.191598241 | 0.150016757 | -7.943100941 | 1.97E-15 | 6.37E-14 | PADI3 |
| 170.58114 | 1.191000554 | 0.168636721 | 7.062522003 | 1.64E-12 | 4.05E-11 | SLC6A9 |
| 47.68639057 | -1.190052651 | 0.267201448 | -4.453765728 | 8.44E-06 | 8.26E-05 | ZNF826P |
| 141.4536326 | -1.188572483 | 0.172609981 | -6.885885007 | 5.74E-12 | 1.35E-10 | TENT5A |
| 3.906205441 | 1.187420144 | 0.344920485 | 3.442590959 | 0.00057617 | 0.003582313 | TFAP2B |
| 7.99486761 | 1.183710585 | 0.366633445 | 3.228594119 | 0.001244003 | 0.007037467 | ALDH1A2 |
| 6.939748515 | -1.18362791 | 0.366307958 | -3.231237229 | 0.001232556 | 0.006977527 | IRAK3 |
| 701.6260621 | 1.182190622 | 0.118175835 | 10.00365787 | 1.47E-23 | 8.21E-22 | IVL |
| 10.33627202 | -1.181917877 | 0.363212621 | -3.254066097 | 0.001137658 | 0.006521432 | GLS2 |
| 147.8346926 | -1.180706678 | 0.210389996 | -5.611990594 | 2.00E-08 | 3.05E-07 | HIST2H2AC |
| 266.6215837 | -1.180351316 | 0.142169212 | -8.30243972 | 1.02E-16 | 3.63E-15 | SLC43A3 |
| 27.27798079 | 1.179729764 | 0.30214401 | 3.904528055 | 9.44E-05 | 0.000731997 | TNNI3 |
| 5369.266183 | 1.179269722 | 0.075802922 | 15.55704826 | 1.43E-54 | 3.71E-52 | INHBA |
| 559.138282 | 1.178124494 | 0.140797832 | 8.3674903 | 5.89E-17 | 2.15E-15 | SLPI |
| 56.78193633 | 1.178109296 | 0.261912024 | 4.498110763 | 6.86E-06 | 6.89E-05 | LCE1B |
| 165.0662592 | 1.177308757 | 0.160363819 | 7.341486175 | 2.11E-13 | 5.68E-12 | FGFR3 |
| 16.86483869 | -1.176298102 | 0.338403722 | -3.476019985 | 0.000508914 | 0.003217887 | CA2 |
| 955.7373791 | 1.176162965 | 0.091908617 | 12.79709133 | 1.70E-37 | 2.05E-35 | HEPHL1 |
| 7.587313232 | 1.176018817 | 0.366449824 | 3.209221942 | 0.001330947 | 0.007447031 | NA |
| 830.009604 | -1.174956714 | 0.088497642 | -13.27670085 | 3.16E-40 | 4.24E-38 | ANXA6 |
| 47.68186433 | 1.174500637 | 0.252435933 | 4.652668197 | 3.28E-06 | 3.50E-05 | CLEC11A |
| 130.4433134 | -1.174093196 | 0.176572669 | -6.649348425 | 2.94E-11 | 6.24E-10 | RAB27B |
| 4.593969435 | 1.172559151 | 0.352326601 | 3.328046043 | 0.000874574 | 0.005163296 | LINC02562 |
| 5.648310862 | 1.17239661 | 0.361671737 | 3.241604169 | 0.00118859 | 0.006768393 | SULT1E1 |
| 16.02231237 | 1.171645931 | 0.343447504 | 3.411426545 | 0.000646239 | 0.003946081 | CFAP57 |
| 8.078792522 | -1.170358616 | 0.366603525 | -3.192436889 | 0.001410778 | 0.007826878 | E2F2 |
| 30.39432908 | 1.170316943 | 0.311988593 | 3.751152986 | 0.000176023 | 0.001270951 | IL23A |
| 40.40427734 | 1.170187524 | 0.267821994 | 4.36927343 | 1.25E-05 | 0.000117307 | TNRC6C |
| 19.17945823 | 1.170099772 | 0.338993358 | 3.451689379 | 0.000557089 | 0.003475546 | KRT1 |
| 8.790023041 | -1.168594478 | 0.366326898 | -3.190031866 | 0.001422571 | 0.007878966 | NA |
| 358.562897 | -1.166720934 | 0.122391755 | -9.532675887 | 1.53E-21 | 7.68E-20 | MCM6 |
| 8.088404921 | 1.165729857 | 0.366058342 | 3.184546623 | 0.00144981 | 0.008000077 | NA |
| 370.5624879 | -1.165266972 | 0.114753998 | -10.15447825 | 3.16E-24 | 1.84E-22 | BRCA1 |
| 70.44001502 | 1.163854841 | 0.220566642 | 5.276658486 | 1.32E-07 | 1.80E-06 | THBS3 |
| 129.7307991 | -1.163774336 | 0.175661919 | -6.625080391 | 3.47E-11 | 7.31E-10 | CMBL |
| 57.30802769 | 1.16321445 | 0.257502387 | 4.517295803 | 6.26E-06 | 6.34E-05 | NEIL1 |
| 4.89664037 | -1.162967472 | 0.355407135 | -3.272211948 | 0.001067095 | 0.00615809 | NA |
| 169.3696881 | -1.162331044 | 0.15590077 | -7.455582431 | 8.95E-14 | 2.49E-12 | RFC5 |
| 25.2918421 | -1.162095622 | 0.329347314 | -3.528480648 | 0.000417953 | 0.00270859 | WDR62 |
| 206.8003958 | -1.162074497 | 0.148262306 | -7.837963192 | 4.58E-15 | 1.42E-13 | ORC6 |
| 5.543351414 | -1.161402332 | 0.352753389 | -3.292391702 | 0.000993391 | 0.005799976 | RGCC |
| 54.31855813 | -1.160622398 | 0.239099953 | -4.854130607 | 1.21E-06 | 1.41E-05 | CD74 |
| 30.59709866 | 1.160137417 | 0.288843496 | 4.016491404 | 5.91E-05 | 0.000479327 | AK5 |
| 3.645681333 | -1.159784361 | 0.340815445 | -3.402968905 | 0.000666579 | 0.004062708 | HDGFP1 |
| 22.2185958 | 1.158882404 | 0.3202257 | 3.618955014 | 0.000295795 | 0.00200414 | CTSK |
| 36.81585878 | -1.157472454 | 0.272968483 | -4.240315368 | 2.23E-05 | 0.000199388 | DNA2 |
| 248.1221721 | 1.156477122 | 0.136503534 | 8.472140518 | 2.41E-17 | 9.09E-16 | TMEM200A |
| 335.5523019 | -1.156171465 | 0.118248632 | -9.77746165 | 1.41E-22 | 7.56E-21 | SAA1 |
| 6.737024736 | -1.153998626 | 0.363875474 | -3.171410853 | 0.001517004 | 0.008306511 | MIR646HG |
| 7.409607809 | -1.152769518 | 0.36659515 | -3.144530191 | 0.001663537 | 0.009012478 | HIF3A |
| 67.62487001 | -1.151526606 | 0.222555863 | -5.174101415 | 2.29E-07 | 3.01E-06 | PSRC1 |
| 22.52487866 | -1.150568795 | 0.322576258 | -3.566811768 | 0.000361351 | 0.002388064 | FOXL1 |
| 14.00213569 | 1.148476522 | 0.359094547 | 3.198256643 | 0.001382612 | 0.007694065 | AMACR |
| 201.7721606 | 1.147551141 | 0.153431722 | 7.479230018 | 7.48E-14 | 2.09E-12 | FNDC4 |
| 33.96290994 | 1.147239888 | 0.294141462 | 3.900299808 | 9.61E-05 | 0.000742791 | GAL |
| 96.21461272 | 1.145792079 | 0.20727402 | 5.527909773 | 3.24E-08 | 4.83E-07 | METRNL |
| 14.848986 | 1.145499679 | 0.353858579 | 3.237168029 | 0.001207223 | 0.006855437 | PAPPA |
| 77.58689889 | -1.145064106 | 0.218784453 | -5.233754467 | 1.66E-07 | 2.23E-06 | CENPI |
| 49.36860103 | -1.144887955 | 0.250085442 | -4.577987209 | 4.69E-06 | 4.86E-05 | SCN4B |
| 8.898419687 | 1.144884215 | 0.365405772 | 3.133185908 | 0.001729199 | 0.009312776 | CD86 |
| 23.55655921 | 1.143738205 | 0.321220269 | 3.560604098 | 0.000370003 | 0.002435417 | BNC2 |
| 29.64382302 | 1.14365561 | 0.293150095 | 3.901262969 | 9.57E-05 | 0.00074054 | GPR68 |
| 4.23376046 | 1.142928152 | 0.35089678 | 3.257163412 | 0.001125316 | 0.006457873 | LINC00923 |
| 4.338589995 | -1.142390127 | 0.34827249 | -3.280161825 | 0.001037476 | 0.006012367 | RPS26P3 |
| 441.7119798 | 1.141540902 | 0.117191156 | 9.740845122 | 2.02E-22 | 1.07E-20 | TUBA1A |
| 5695.769188 | 1.141453537 | 0.09525075 | 11.98366985 | 4.33E-33 | 4.10E-31 | SPRR1B |
| 22.23583768 | 1.141176346 | 0.326564188 | 3.494493238 | 0.000474963 | 0.003037203 | GSDMB |
| 19.18164618 | 1.140972379 | 0.328044249 | 3.478105107 | 0.000504972 | 0.003196659 | WNT3 |
| 7.64481045 | -1.140791124 | 0.366546875 | -3.112265315 | 0.001856576 | 0.009888257 | PAQR5 |
| 63.24835709 | -1.140318856 | 0.230245312 | -4.952625736 | 7.32E-07 | 8.84E-06 | TEDC1 |
| 186.7627403 | -1.14023235 | 0.151653584 | -7.51866407 | 5.53E-14 | 1.58E-12 | LNCAROD |
| 578.1190238 | -1.139833157 | 0.106476707 | -10.70500012 | 9.64E-27 | 6.47E-25 | HMGB2 |
| 4.057770347 | -1.138115939 | 0.348896435 | -3.262045198 | 0.001106115 | 0.006367352 | SUSD5 |
| 56.01571744 | 1.137016667 | 0.252979343 | 4.49450399 | 6.97E-06 | 7.00E-05 | CRYAB |
| 302.170216 | -1.136247267 | 0.154608323 | -7.349198561 | 1.99E-13 | 5.39E-12 | H19 |
| 15.1836841 | -1.134809064 | 0.342075694 | -3.317420926 | 0.000908526 | 0.005344483 | TPK1 |
| 330.6442517 | -1.133947465 | 0.126836275 | -8.94024576 | 3.88E-19 | 1.63E-17 | KIF23 |
| 29.56205561 | 1.133389226 | 0.296696483 | 3.82002919 | 0.000133436 | 0.000990521 | PGGHG |
| 28.7753477 | -1.133363806 | 0.29244917 | -3.875421523 | 0.00010644 | 0.000810321 | TRAIP |
| 2107.370978 | 1.132918487 | 0.099306631 | 11.40828644 | 3.80E-30 | 3.02E-28 | UPP1 |
| 149.8260231 | -1.132315181 | 0.168482047 | -6.720687445 | 1.81E-11 | 3.94E-10 | KIF15 |
| 3.784794772 | 1.132046525 | 0.341286734 | 3.316995396 | 0.000909911 | 0.005350709 | CTNND2 |
| 207.594822 | -1.131015533 | 0.145022154 | -7.798915582 | 6.24E-15 | 1.90E-13 | PHYHD1 |
| 305.2844192 | 1.130985308 | 0.123249427 | 9.176394037 | 4.46E-20 | 2.01E-18 | IQCA1 |
| 53.51834962 | 1.130947664 | 0.254700236 | 4.44030866 | 8.98E-06 | 8.73E-05 | RAET1E |
| 232.6538256 | 1.128052613 | 0.138670985 | 8.134741464 | 4.13E-16 | 1.41E-14 | RASSF6 |
| 44.1388473 | 1.127999441 | 0.263287764 | 4.284283571 | 1.83E-05 | 0.00016686 | IGFL2 |
| 402.3981609 | -1.12731928 | 0.117011517 | -9.634259186 | 5.73E-22 | 2.94E-20 | AFAP1 |
| 6.463945315 | 1.126771919 | 0.365721827 | 3.080953437 | 0.002063389 | 0.010813981 | CYP2E1 |
| 1880.54812 | -1.126529503 | 0.084395081 | -13.34828401 | 1.21E-40 | 1.68E-38 | RRM1 |
| 281.206243 | -1.126424522 | 0.124953796 | -9.014728294 | 1.97E-19 | 8.49E-18 | GPR153 |
| 108.6515686 | -1.126094082 | 0.182020292 | -6.186640339 | 6.15E-10 | 1.13E-08 | XRRA1 |
| 114.7364911 | -1.125258669 | 0.191031565 | -5.890433177 | 3.85E-09 | 6.50E-08 | FANCG |
| 16.26235857 | 1.124996245 | 0.343305651 | 3.276952305 | 0.001049341 | 0.006070807 | RAET1G |
| 588.8072745 | 1.124932561 | 0.106585902 | 10.55423407 | 4.86E-26 | 3.11E-24 | GNAL |
| 249.0262044 | 1.123759015 | 0.146739536 | 7.658188419 | 1.89E-14 | 5.52E-13 | NFKBIZ |
| 1323.220017 | -1.123052111 | 0.083167218 | -13.50354305 | 1.49E-41 | 2.14E-39 | PRR11 |
| 3.990272107 | -1.122665677 | 0.347881074 | -3.227153652 | 0.001250283 | 0.007060132 | RNU1-122P |
| 372.1620506 | -1.122615164 | 0.1220776 | -9.19591444 | 3.72E-20 | 1.70E-18 | BARD1 |
| 11.00619475 | -1.122614309 | 0.362923378 | -3.093254328 | 0.001979744 | 0.010428012 | CEL |
| 77.09615181 | -1.12217499 | 0.214573453 | -5.229794152 | 1.70E-07 | 2.28E-06 | MRM3 |
| 358.4887477 | 1.121906708 | 0.122107264 | 9.187878516 | 4.01E-20 | 1.82E-18 | SLC1A4 |
| 8.33281794 | 1.121324778 | 0.366437 | 3.060075204 | 0.002212814 | 0.011497791 | APCDD1L-DT |
| 13.78471621 | 1.120865973 | 0.348681958 | 3.214579786 | 0.001306356 | 0.00732194 | SMPDL3B |
| 12.95094029 | -1.119970028 | 0.356598024 | -3.140707327 | 0.001685404 | 0.009112861 | TMPO-AS1 |
| 98.92197041 | -1.119835383 | 0.198054504 | -5.654177822 | 1.57E-08 | 2.43E-07 | HAUS8 |
| 498.2780731 | 1.118907056 | 0.111458189 | 10.03880529 | 1.03E-23 | 5.79E-22 | PTPRK |
| 12.72086686 | 1.118640889 | 0.352724476 | 3.171429725 | 0.001516906 | 0.008306511 | GRIN2D |
| 29.98595721 | -1.117522755 | 0.290279166 | -3.849820743 | 0.000118204 | 0.000887507 | CNIH3 |
| 8.584237897 | 1.117377123 | 0.365328609 | 3.058553572 | 0.002224083 | 0.011541702 | GCNA |
| 134.5969387 | -1.11712152 | 0.170779949 | -6.541292052 | 6.10E-11 | 1.25E-09 | SLC37A2 |
| 10.50148372 | -1.116774104 | 0.359924083 | -3.102804613 | 0.001916962 | 0.010143922 | KIAA1614- |
| | | | | | | AS1 |
| 4.086015025 | -1.116405209 | 0.347196742 | -3.215482963 | 0.001302252 | 0.007303937 | MAP2K6 |
| 4.283626928 | 1.11531508 | 0.349812613 | 3.18832151 | 0.001431013 | 0.007920302 | GABRA3 |
| 33.12238019 | -1.114323688 | 0.287220043 | -3.879686376 | 0.000104591 | 0.000797727 | ZNF90 |
| 18.5478728 | -1.114029795 | 0.341585353 | -3.26135118 | 0.001108826 | 0.006376237 | IL15RA |
| 62.19602554 | -1.113725657 | 0.227028403 | -4.905666621 | 9.31E-07 | 1.10E-05 | DLEU1 |
| 203.3007417 | 1.110851972 | 0.143992031 | 7.714676766 | 1.21E-14 | 3.62E-13 | WNT5A |
| 691.4495322 | -1.109849208 | 0.108157669 | -10.26140092 | 1.05E-24 | 6.29E-23 | NRP2 |
| 6261.205837 | 1.109305428 | 0.069356729 | 15.99420047 | 1.40E-57 | 4.03E-55 | PHLDA1 |
| 14.73729954 | -1.109272265 | 0.350431528 | -3.16544653 | 0.001548451 | 0.008459362 | CAMK2B |
| 11.89009169 | -1.109043318 | 0.359386673 | -3.085933346 | 0.002029143 | 0.010651538 | CRNN |
| 18.09974898 | 1.108706302 | 0.332242391 | 3.337040463 | 0.000846756 | 0.005011708 | NA |
| 10.31620899 | 1.107792041 | 0.362377269 | 3.057013056 | 0.002235545 | 0.011590171 | SEMA6A |
| 23.10934272 | -1.107492721 | 0.315348399 | -3.511965574 | 0.000444806 | 0.00286336 | CYSRT1 |
| 5.407785996 | 1.106756945 | 0.360198347 | 3.072631936 | 0.0021218 | 0.011088182 | SULF1 |
| 4.401425792 | -1.105546593 | 0.354288524 | -3.120469666 | 0.001805629 | 0.009660881 | NA |
| 227.8707927 | -1.105026225 | 0.13645535 | -8.098079177 | 5.58E-16 | 1.88E-14 | HERC5 |
| 4.293602515 | 1.103757683 | 0.34899724 | 3.162654473 | 0.001563378 | 0.008531918 | GDA |
| 6.474996047 | 1.102574778 | 0.364846726 | 3.022021846 | 0.002510924 | 0.012791192 | NUDT16P1 |
| 596.5161224 | -1.102453475 | 0.305913836 | -3.603803901 | 0.000313594 | 0.002110766 | RNU4ATAC |
| 61.06336934 | -1.100983162 | 0.234325271 | -4.69852507 | 2.62E-06 | 2.85E-05 | DHFR |
| 7.925646452 | 1.100846509 | 0.366463007 | 3.003977176 | 0.002664753 | 0.01346181 | RNFT2 |
| 23.19402302 | 1.099338226 | 0.314721861 | 3.493046919 | 0.000477543 | 0.003049002 | ZFP3 |
| 17.93460815 | 1.098465078 | 0.332956344 | 3.299126439 | 0.000969862 | 0.005672707 | C1orf210 |
| 9.750916719 | 1.098015271 | 0.364240605 | 3.01453286 | 0.002573753 | 0.013072729 | NA |
| 76.09017777 | 1.097992772 | 0.230428084 | 4.765012822 | 1.89E-06 | 2.11E-05 | GABRE |
| 2301.245347 | 1.097145117 | 0.079582011 | 13.78634577 | 3.08E-43 | 4.76E-41 | HLA-A |
| 18.02260945 | 1.096854724 | 0.336753382 | 3.257145382 | 0.001125388 | 0.006457873 | SOCS2-AS1 |
| 36.78890695 | 1.096832854 | 0.278092318 | 3.944132148 | 8.01E-05 | 0.000630517 | NA |
| 190.0382203 | -1.096621838 | 0.162333068 | -6.755381707 | 1.42E-11 | 3.16E-10 | UBE2T |
| 64.70209546 | -1.096225071 | 0.235980226 | -4.645410713 | 3.39E-06 | 3.61E-05 | TNS2 |
| 241.9474572 | 1.095984848 | 0.152569032 | 7.183534132 | 6.79E-13 | 1.74E-11 | HOTAIRM1 |
| 39.94782505 | 1.095903094 | 0.270663483 | 4.048950677 | 5.14E-05 | 0.000424192 | ULK4 |
| 18.63775483 | 1.09552548 | 0.335974682 | 3.260738195 | 0.001111226 | 0.006387794 | NA |
| 565.491649 | 1.094585909 | 0.107563046 | 10.1762264 | 2.53E-24 | 1.48E-22 | SERPINB7 |
| 493.3318756 | -1.094574559 | 0.112757976 | -9.707291639 | 2.81E-22 | 1.47E-20 | DIAPH3 |
| 45.47601634 | -1.094437449 | 0.259543823 | -4.216773249 | 2.48E-05 | 0.000219109 | TROAP |
| 772.3959168 | 1.093351058 | 0.110267301 | 9.915460379 | 3.57E-23 | 1.97E-21 | WSB1 |
| 24.75538921 | -1.092926386 | 0.306466951 | -3.566212879 | 0.000362177 | 0.002391596 | PRRT3-AS1 |
| 1110.090444 | -1.092788 | 0.087394818 | -12.50403656 | 7.10E-36 | 7.91E-34 | PTTG1 |
| 32.87471884 | -1.092009788 | 0.302164273 | -3.613960631 | 0.000301555 | 0.002035589 | ESPL1 |
| 327.9674622 | -1.09155303 | 0.118547719 | -9.20771007 | 3.33E-20 | 1.52E-18 | CYB5A |
| 4.85522516 | 1.091344356 | 0.355026633 | 3.073978837 | 0.002112244 | 0.011048817 | LINC00894 |
| 64860.29683 | 1.090437203 | 0.098258776 | 11.09760622 | 1.29E-28 | 9.60E-27 | MALAT1 |
| 36.44299862 | 1.090087693 | 0.282128386 | 3.863800129 | 0.000111637 | 0.000843608 | LMF1 |
| 184.9783383 | -1.088691619 | 0.149589051 | -7.277883035 | 3.39E-13 | 8.93E-12 | DEPDC1B |
| 16.93827942 | 1.088478559 | 0.337923649 | 3.221078375 | 0.001277092 | 0.007180028 | PBX4 |
| 144.9880208 | -1.088215381 | 0.173943453 | -6.256144534 | 3.95E-10 | 7.39E-09 | KRBOX1 |
| 283.6181124 | -1.087280322 | 0.161170819 | -6.746136352 | 1.52E-11 | 3.35E-10 | PDPR |
| 3.833368369 | -1.0869553 | 0.346009409 | -3.141403881 | 0.0016814 | 0.009097217 | NA |
| 10.71787003 | 1.086323325 | 0.363950423 | 2.984811272 | 0.002837533 | 0.014190265 | CHMP1B2P |
| 91.40595107 | 1.085559229 | 0.196521894 | 5.523858992 | 3.32E-08 | 4.93E-07 | CTSS |
| 35.21857398 | 1.085239685 | 0.280111331 | 3.874315549 | 0.000106925 | 0.000813254 | NBPF8 |
| 3.518959438 | -1.085091492 | 0.335283797 | -3.236337398 | 0.001210742 | 0.006865899 | NA |
| 1067.876374 | -1.085047521 | 0.105425732 | -10.29205584 | 7.65E-25 | 4.59E-23 | CENPE |
| 84.86369233 | -1.084440776 | 0.208625596 | -5.19802362 | 2.01E-07 | 2.67E-06 | PAX8-AS1 |
| 22.99204449 | -1.083231623 | 0.321593139 | -3.368329392 | 0.000756252 | 0.00452839 | RAD54B |
| 376.1187743 | -1.083220571 | 0.116839773 | -9.27099178 | 1.84E-20 | 8.66E-19 | ITGB3BP |
| 60.35831333 | -1.081999696 | 0.231764733 | -4.668526065 | 3.03E-06 | 3.27E-05 | MTFR2 |
| 15.05964672 | 1.081931748 | 0.346748266 | 3.120222523 | 0.001807145 | 0.009665834 | PYROXD2 |
| 24.36655648 | -1.081590834 | 0.312557991 | -3.460448515 | 0.000539276 | 0.003387127 | RPS26P13 |
| 47.26340852 | 1.081375665 | 0.257399027 | 4.201164537 | 2.66E-05 | 0.000233017 | MT1F |
| 567.2985222 | -1.080888151 | 0.106413058 | -10.15747668 | 3.07E-24 | 1.79E-22 | CPT1A |
| 29.42120214 | -1.079039685 | 0.31166432 | -3.462185489 | 0.000535808 | 0.00336849 | HSD17B14 |
| 5.781107214 | 1.078964256 | 0.361452543 | 2.985078612 | 0.002835055 | 0.01418653 | PCSK5 |
| 221.1500958 | -1.078161899 | 0.144073756 | -7.48340247 | 7.24E-14 | 2.04E-12 | ANPEP |
| 151.5716396 | -1.076865997 | 0.170375716 | -6.32053687 | 2.61E-10 | 4.99E-09 | RMI2 |
| 99.18194249 | 1.076403821 | 0.194387901 | 5.537401326 | 3.07E-08 | 4.58E-07 | LZTS3 |
| 316.3512976 | 1.075953251 | 0.128519617 | 8.371899013 | 5.67E-17 | 2.07E-15 | ROBO3 |
| 168.3438078 | -1.074754814 | 0.156346968 | -6.874164729 | 6.24E-12 | 1.46E-10 | GEN1 |
| 4.514511158 | 1.074721257 | 0.353924929 | 3.03657971 | 0.002392788 | 0.012292584 | HSPB8 |
| 285.0059226 | -1.074388067 | 0.134866073 | -7.966333163 | 1.63E-15 | 5.32E-14 | NUDT15 |
| 73.9976225 | 1.07413776 | 0.248369146 | 4.324763268 | 1.53E-05 | 0.000140698 | RHCG |
| 83.9133296 | 1.073973978 | 0.204428927 | 5.253532325 | 1.49E-07 | 2.02E-06 | ITM2A |
| 3.835239026 | -1.073607886 | 0.345010806 | -3.111809451 | 0.001859445 | 0.009898176 | NA |
| 585.9920271 | -1.073195785 | 0.108645104 | -9.877994924 | 5.19E-23 | 2.82E-21 | PPT1 |
| 17.54709142 | -1.073174797 | 0.331433517 | -3.237979084 | 0.001203796 | 0.006843094 | NA |
| 8.705224417 | -1.072441046 | 0.36485169 | -2.939389004 | 0.0032886 | 0.015992027 | HECW2-AS1 |
| 1894.469937 | 1.07240368 | 0.077400693 | 13.85522063 | 1.18E-43 | 1.88E-41 | SMURF2 |
| 46.91797534 | -1.072029304 | 0.25289604 | -4.239011826 | 2.25E-05 | 0.00020044 | SCLY |
| 3055.306265 | 1.071272285 | 0.093083756 | 11.5086921 | 1.19E-30 | 9.67E-29 | NEAT1 |
| 28.3291749 | -1.07019971 | 0.307955103 | -3.475180958 | 0.000510509 | 0.003226723 | LAMP3 |
| 355.732424 | -1.070139345 | 0.120279985 | -8.897069178 | 5.73E-19 | 2.37E-17 | WDHD1 |
| 75.46092136 | 1.06923253 | 0.214312818 | 4.989120771 | 6.07E-07 | 7.43E-06 | VLDLR |
| 13.41777491 | 1.067496246 | 0.352481359 | 3.028518298 | 0.002457562 | 0.012578019 | TMEM81 |
| 374.7844838 | -1.066177071 | 0.119327731 | -8.934864187 | 4.08E-19 | 1.70E-17 | CCDC18 |
| 246.6523689 | -1.066168496 | 0.133707201 | -7.973904841 | 1.54E-15 | 5.03E-14 | TTC39C |
| 6.537889331 | 1.064845253 | 0.36420467 | 2.923755073 | 0.003458367 | 0.016688788 | PCSK1N |
| 421.0723271 | 1.064739301 | 0.116508635 | 9.138715788 | 6.32E-20 | 2.81E-18 | ZNF185 |
| 64.03004154 | 1.064478813 | 0.234074049 | 4.547615665 | 5.43E-06 | 5.56E-05 | SMARCD3 |
| 6.018388733 | 1.062884246 | 0.364975025 | 2.912210898 | 0.003588802 | 0.017236983 | NA |
| 22.93018493 | 1.062635566 | 0.323442409 | 3.285393433 | 0.001018401 | 0.005926983 | CHST10 |
| 351.5732139 | 1.062599427 | 0.132446872 | 8.022835188 | 1.03E-15 | 3.41E-14 | LARP6 |
| 18.29141438 | -1.061565357 | 0.328008918 | -3.236391755 | 0.001210511 | 0.006865899 | SNCG |
| 5.560164726 | 1.060357559 | 0.357750937 | 2.963954663 | 0.00303713 | 0.01498714 | MDGA1 |
| 1163.303906 | -1.060350097 | 0.081895803 | -12.94755115 | 2.43E-38 | 3.06E-36 | NASP |
| 209.8527768 | -1.060246679 | 0.142541712 | -7.438150327 | 1.02E-13 | 2.81E-12 | CAVIN2 |
| 635.5788645 | 1.05867191 | 0.101553596 | 10.42476048 | 1.91E-25 | 1.16E-23 | MRC2 |
| 73.6210676 | -1.058400309 | 0.216753473 | -4.882968168 | 1.05E-06 | 1.23E-05 | SPAG5 |
| 9.478123313 | 1.058330452 | 0.363717878 | 2.909756477 | 0.003617105 | 0.01735257 | WNT10A |
| 3.27178656 | -1.058302278 | 0.333699203 | -3.171425849 | 0.001516926 | 0.008306511 | SENCR |
| 254.3088834 | -1.058269523 | 0.133555082 | -7.92384316 | 2.30E-15 | 7.35E-14 | TMEM164 |
| 94.52087913 | 1.057899776 | 0.202349675 | 5.228077456 | 1.71E-07 | 2.29E-06 | PLEKHO1 |
| 506.572828 | 1.057597898 | 0.110026772 | 9.612186909 | 7.10E-22 | 3.62E-20 | ABCA1 |
| 28.44617041 | 1.057267185 | 0.302687123 | 3.492937445 | 0.000477738 | 0.003049002 | RHBDL1 |
| 19.11323463 | 1.056072674 | 0.33478196 | 3.15450891 | 0.001607685 | 0.008744588 | CCDC88B |
| 16.36180329 | -1.055871066 | 0.347060531 | -3.042325389 | 0.00234758 | 0.012086863 | NA |
| 7.112164841 | 1.05548348 | 0.366516851 | 2.879767949 | 0.00397968 | 0.01878395 | AKR1E2 |
| 230.2172425 | -1.055471724 | 0.142956602 | -7.383161798 | 1.55E-13 | 4.21E-12 | ENOSF1 |
| 44.06296829 | -1.0525134 | 0.257067417 | -4.094308853 | 4.23E-05 | 0.000356111 | TXNDC16 |
| 136.7221255 | -1.051944331 | 0.172076648 | -6.113231182 | 9.76E-10 | 1.75E-08 | CCDC88C |
| 202.1680517 | -1.05114205 | 0.141414166 | -7.43307463 | 1.06E-13 | 2.91E-12 | RFC4 |
| 763.21636 | 1.05082024 | 0.095333453 | 11.02257615 | 2.97E-28 | 2.18E-26 | CCDC80 |
| 18.40708084 | 1.048985833 | 0.329861701 | 3.180077681 | 0.001472356 | 0.008113557 | LHX5 |
| 4.804942212 | -1.048756177 | 0.356503565 | -2.94178314 | 0.003263284 | 0.015897228 | LINC01117 |
| 25.78523112 | 1.048169012 | 0.31314523 | 3.347229687 | 0.000816235 | 0.004853861 | TPPP |
| 7.888519178 | -1.047557985 | 0.3666511 | -2.857097617 | 0.004275343 | 0.019960943 | ZNF695 |
| 155.4388274 | -1.046834289 | 0.157074179 | -6.664585444 | 2.65E-11 | 5.69E-10 | NID1 |
| 58.5538766 | 1.046262317 | 0.234905976 | 4.453962116 | 8.43E-06 | 8.26E-05 | CCDC18-A51 |
| 288.7148003 | 1.046124135 | 0.128393874 | 8.147772962 | 3.71E-16 | 1.28E-14 | SETBP1 |
| 16.97689572 | -1.045771821 | 0.338367828 | -3.09063609 | 0.001997283 | 0.010504488 | UBA7 |
| 71.93609383 | 1.045619733 | 0.219855463 | 4.755941552 | 1.98E-06 | 2.20E-05 | RNF180 |
| 30.29474657 | 1.045287766 | 0.289067776 | 3.616064652 | 0.000299116 | 0.002024127 | IL11 |
| 223.4247735 | 1.044921549 | 0.149494447 | 6.989701415 | 2.75E-12 | 6.73E-11 | PYROXD1 |
| 14.8751097 | 1.044148702 | 0.345122137 | 3.025446911 | 0.00248266 | 0.012670846 | LINC00649 |
| 815.2069991 | 1.043992173 | 0.097054777 | 10.75673147 | 5.51E-27 | 3.73E-25 | MYADM |
| 125.7868768 | 1.043480192 | 0.176488641 | 5.912449568 | 3.37E-09 | 5.73E-08 | ECM1 |
| 141.8745152 | 1.043365397 | 0.165909873 | 6.288748096 | 3.20E-10 | 6.06E-09 | DUOXA1 |
| 12.07693944 | -1.043101548 | 0.355881648 | -2.931034948 | 0.003378348 | 0.016370148 | NA |
| 864.0941425 | -1.042053646 | 0.088797208 | -11.73520734 | 8.41E-32 | 7.25E-30 | TMPC |
| 67047.45932 | 1.041113524 | 0.060262482 | 17.27631328 | 7.09E-67 | 2.83E-64 | LAMC2 |
| 221.7545565 | 1.040901417 | 0.142001747 | 7.330201475 | 2.30E-13 | 6.16E-12 | TMC6 |
| 138.4137836 | 1.040575586 | 0.170009264 | 6.120699322 | 9.32E-10 | 1.67E-08 | RHBDL2 |
| 120.9204365 | 1.040481441 | 0.18289607 | 5.688921802 | 1.28E-08 | 2.01E-07 | ARHGAP31 |
| 119.8607159 | -1.039540684 | 0.180193121 | -5.76903644 | 7.97E-09 | 1.30E-07 | NRM |
| 266.875406 | -1.039292734 | 0.138246633 | -7.517671237 | 5.58E-14 | 1.59E-12 | HS3ST2 |
| 86.28554882 | 1.039199171 | 0.19959001 | 5.206669255 | 1.92E-07 | 2.55E-06 | FANK1 |
| 259.7015144 | 1.039191371 | 0.138383866 | 7.509483605 | 5.94E-14 | 1.68E-12 | PRDM1 |
| 38.32954385 | -1.039013638 | 0.27696091 | -3.751481164 | 0.000175793 | 0.001269848 | SNORA12 |
| 1538.877129 | -1.038587437 | 0.079911396 | -12.99673748 | 1.28E-38 | 1.63E-36 | SMC2 |
| 317.0337692 | -1.038554914 | 0.131053484 | -7.924664651 | 2.29E-15 | 7.32E-14 | CENPQ |
| 13.68805935 | -1.037186751 | 0.346342653 | -2.994683856 | 0.002747294 | 0.013831875 | NA |
| 618.9212743 | 1.036991604 | 0.101672911 | 10.19929103 | 2.00E-24 | 1.18E-22 | PYCR1 |
| 10.34813076 | 1.036613757 | 0.364721518 | 2.842206189 | 0.004480251 | 0.020735239 | RASGRP2 |
| 18.76761109 | -1.035259818 | 0.330052113 | -3.136655627 | 0.001708867 | 0.009225804 | DNM3 |
| 189.302859 | -1.034840623 | 0.147917779 | -6.996053032 | 2.63E-12 | 6.45E-11 | SLFN12 |
| 191.0871748 | 1.034745452 | 0.147460495 | 7.017102791 | 2.27E-12 | 5.58E-11 | ID1 |
| 54.79580366 | 1.034087757 | 0.265555619 | 3.89405338 | 9.86E-05 | 0.000758612 | NA |
| 3.40154955 | 1.033750197 | 0.334282958 | 3.092440618 | 0.001985179 | 0.010450898 | ESPNL |
| 18.20229314 | -1.033007357 | 0.33058579 | -3.124778461 | 0.00177939 | 0.009554669 | KIF24 |
| 28.8495746 | 1.032406661 | 0.297125213 | 3.474651821 | 0.000511517 | 0.003231847 | NA |
| 72.78088718 | 1.032219106 | 0.21901108 | 4.713090797 | 2.44E-06 | 2.67E-05 | ASIC1 |
| 51.06548979 | -1.032152712 | 0.243311354 | -4.242106639 | 2.21E-05 | 0.000198019 | SUV39H1 |
| 112.6137851 | -1.032134799 | 0.185426544 | -5.566273175 | 2.60E-08 | 3.92E-07 | DYSF |
| 179.9930537 | -1.032032176 | 0.153467201 | -6.724773575 | 1.76E-11 | 3.84E-10 | SESN1 |
| 131.8496759 | 1.030213594 | 0.194589891 | 5.294281149 | 1.19E-07 | 1.64E-06 | PLPPR2 |
| 886.1631323 | 1.029952928 | 0.101872688 | 10.11019685 | 4.98E-24 | 2.88E-22 | C1orf74 |
| 42.01840445 | 1.02988227 | 0.266057916 | 3.870895044 | 0.000108436 | 0.000822461 | LCE1E |
| 197.9263827 | -1.029457827 | 0.142896071 | -7.20424166 | 5.84E-13 | 1.50E-11 | UBR7 |
| 193.6978861 | -1.02935952 | 0.150883203 | -6.822227391 | 8.96E-12 | 2.06E-10 | RAB7B |
| 759.1167252 | 1.028631834 | 0.096313762 | 10.68000893 | 1.26E-26 | 8.34E-25 | MAOA |
| 54.07709016 | -1.027862066 | 0.243337285 | -4.22402208 | 2.40E-05 | 0.000212749 | CHTF18 |
| 5.620912162 | -1.027754629 | 0.36309948 | -2.830504272 | 0.004647469 | 0.021375487 | HIST1H2BM |
| 167.0236604 | 1.027582621 | 0.162756887 | 6.313604557 | 2.73E-10 | 5.19E-09 | GOLGA8B |
| 535.4755993 | -1.027306656 | 0.107832787 | -9.526848768 | 1.62E-21 | 8.10E-20 | GSR |
| 188.8134248 | 1.026999888 | 0.174870883 | 5.872903865 | 4.28E-09 | 7.17E-08 | PPP2R5B |
| 58.85541071 | 1.025197662 | 0.239532908 | 4.279986702 | 1.87E-05 | 0.000169831 | LURAP1L |
| 5.833281619 | 1.024892386 | 0.359067498 | 2.854316783 | 0.00431295 | 0.020125053 | CACNA2D1 |
| 3.291645814 | 1.024511251 | 0.333827932 | 3.068980014 | 0.00214791 | 0.011203185 | PPBP |
| 319.5109983 | -1.024464315 | 0.12410429 | -8.254866253 | 1.52E-16 | 5.37E-15 | LINC00665 |
| 57.79244343 | -1.024280191 | 0.237351286 | -4.315460887 | 1.59E-05 | 0.000146345 | CARD16 |
| 17.27689214 | -1.023718517 | 0.339996675 | -3.010966258 | 0.002604178 | 0.013184254 | FRGIJP |
| 111.0162775 | 1.023190421 | 0.205257951 | 4.984900302 | 6.20E-07 | 7.57E-06 | KIFC2 |
| 87.81998949 | 1.022917349 | 0.216694443 | 4.720551836 | 2.35E-06 | 2.59E-05 | GABBR1 |
| 14.37582888 | -1.022898524 | 0.34811446 | -2.938397116 | 0.003299141 | 0.016033768 | C5 |
| 8.478188004 | 1.022492803 | 0.36501438 | 2.801239784 | 0.005090668 | 0.023102607 | DSC1 |
| 27.37416794 | -1.022216435 | 0.295019986 | -3.464905713 | 0.000530417 | 0.003339673 | NA |
| 183.7928292 | 1.021421888 | 0.311332351 | 3.280808709 | 0.001035099 | 0.006002843 | LCE3D |
| 4.722175391 | 1.020963741 | 0.358067217 | 2.851318669 | 0.004353831 | 0.020275388 | IL37 |
| 3.771760136 | 1.019892922 | 0.345022361 | 2.956019774 | 0.003116371 | 0.015295239 | NA |
| 184.2690947 | 1.019699727 | 0.171896099 | 5.932070205 | 2.99E-09 | 5.11E-08 | MELTF |
| 207.2226852 | 1.019395056 | 0.152473752 | 6.685708488 | 2.30E-11 | 4.96E-10 | SERPINB13 |
| 1800.522954 | 1.018338849 | 0.082294942 | 12.37425801 | 3.60E-35 | 3.76E-33 | TMEM154 |
| 63.2520462 | -1.018220269 | 0.249279509 | -4.084652895 | 4.41E-05 | 0.00036954 | CMPK2 |
| 129.6616073 | -1.018191587 | 0.175367163 | -5.806056116 | 6.40E-09 | 1.05E-07 | ARL4A |
| 6.565541172 | -1.017897617 | 0.365965813 | -2.781400829 | 0.005412486 | 0.024194479 | DAPL1 |
| 58.19726979 | -1.0168268 | 0.229697584 | -4.426806681 | 9.56E-06 | 9.24E-05 | HOXD-AS2 |
| 275.0909719 | -1.01641649 | 0.137174183 | -7.409677764 | 1.27E-13 | 3.47E-12 | IRF1 |
| 9.390555217 | -1.016250035 | 0.364150949 | -2.790738395 | 0.005258796 | 0.023694953 | NA |
| 185.4523567 | -1.0161691 | 0.159043749 | -6.389242613 | 1.67E-10 | 3.24E-09 | MX2 |
| 10.80083972 | -1.015775718 | 0.359126664 | -2.828460874 | 0.004677242 | 0.021488293 | NA |
| 220.9454753 | -1.015763171 | 0.14298854 | -7.103808282 | 1.21E-12 | 3.04E-11 | SLC29A1 |
| 132.0428796 | -1.015667608 | 0.172314212 | -5.894276483 | 3.76E-09 | 6.36E-08 | FMN1 |
| 30.57655431 | 1.015429984 | 0.290355622 | 3.49719415 | 0.00047018 | 0.003008965 | FAM171A2 |
| 90.77864692 | 1.014697477 | 0.203051796 | 4.997234675 | 5.82E-07 | 7.15E-06 | UPK1B |
| 146.4542942 | 1.014006018 | 0.186138411 | 5.447591466 | 5.11E-08 | 7.44E-07 | MAPK12 |
| 7.91503683 | -1.013655026 | 0.366347749 | -2.766920305 | 0.005658859 | 0.025050503 | NA |
| 63.64461087 | -1.013264374 | 0.229029027 | -4.424174466 | 9.68E-06 | 9.32E-05 | NA |
| 8.940500898 | 1.013260909 | 0.364123421 | 2.782740275 | 0.005390194 | 0.024140936 | AURKC |
| 54.62505397 | 1.013224576 | 0.245568238 | 4.126040834 | 3.69E-05 | 0.00031475 | RUNDC3A- |
| | | | | | | AS1 |
| 15.73114391 | -1.013109084 | 0.342641305 | -2.95676286 | 0.003108871 | 0.015263002 | NA |
| 3.287040857 | 1.012847841 | 0.33278722 | 3.043529858 | 0.002338202 | 0.012042367 | TDRD1 |
| 423.4817774 | 1.012508949 | 0.112446546 | 9.004357947 | 2.17E-19 | 9.28E-18 | CCDC92 |
| 17.1633943 | 1.012311698 | 0.34297576 | 2.951554643 | 0.003161786 | 0.015457935 | MFAP3L |
| 13.8748548 | -1.011865063 | 0.350386984 | -2.887850037 | 0.003878848 | 0.018387488 | BTN3A3 |
| 5.037508523 | -1.011369994 | 0.359315383 | -2.814713873 | 0.004882068 | 0.022310438 | LINC00518 |
| 1292.819365 | 1.011260532 | 0.116227751 | 8.700680574 | 3.30E-18 | 1.32E-16 | CD24 |
| 40.57252323 | 1.010888962 | 0.276067114 | 3.661750752 | 0.000250498 | 0.001727231 | CMTM8 |
| 160.1137437 | 1.010617959 | 0.157715076 | 6.407871596 | 1.48E-10 | 2.89E-09 | FCGRT |
| 13.60116183 | -1.010551692 | 0.348740459 | -2.897718535 | 0.003758878 | 0.017931534 | NA |
| 4.773325915 | -1.010375671 | 0.354172609 | -2.852777561 | 0.004333895 | 0.020188284 | RTCA-AS1 |
| 2408.975392 | -1.010133335 | 0.071750122 | -14.07848953 | 5.15E-45 | 8.88E-43 | TFPI2 |
| 29.63867532 | -1.009874785 | 0.291776271 | -3.461127191 | 0.000537919 | 0.003380407 | SNHG26 |
| 6.723358935 | -1.009597406 | 0.365263156 | -2.764027495 | 0.005709273 | 0.025238806 | NA |
| 11.47395663 | 1.009416172 | 0.360878947 | 2.797104626 | 0.005156283 | 0.023348643 | BICDL2 |
| 202.6503912 | -1.009258846 | 0.148562672 | -6.793488773 | 1.09E-11 | 2.48E-10 | MRI1 |
| 95.57007432 | -1.00898385 | 0.20123882 | -5.013862872 | 5.33E-07 | 6.58E-06 | MYBL1 |
| 1701.099621 | 1.007678056 | 0.07951801 | 12.67232493 | 8.42E-37 | 9.78E-35 | DLG1 |
| 8.914881087 | 1.007196049 | 0.364312448 | 2.764649015 | 0.005698407 | 0.025204376 | SPRR4 |
| 30.85397041 | 1.00719405 | 0.293273324 | 3.434318668 | 0.000594045 | 0.003677627 | COL13A1 |
| 17.03572567 | -1.007159301 | 0.348416503 | -2.890676225 | 0.003844139 | 0.018259941 | SNORA31 |
| 5.201524039 | 1.006430382 | 0.361706234 | 2.782452406 | 0.005394978 | 0.024149159 | NA |
| 15.82037731 | -1.006295883 | 0.339042098 | -2.968055851 | 0.002996899 | 0.01483329 | E2F8 |
| 16784.30701 | 1.003990295 | 0.077849482 | 12.89655714 | 4.71E-38 | 5.80E-36 | LAMA3 |
| 14.10210352 | 1.00298529 | 0.348860376 | 2.875033562 | 0.004039846 | 0.019007694 | EGFL8 |
| 36.63618482 | 1.002584723 | 0.306705472 | 3.268884365 | 0.001079724 | 0.006224181 | GGT7 |
| 7.294974936 | 1.002267122 | 0.36665248 | 2.733561551 | 0.006265341 | 0.027357432 | SEC31B |
| 93.58946732 | -1.001489346 | 0.199517257 | -5.019562512 | 5.18E-07 | 6.41E-06 | C3 |
| 1034.791179 | -1.001457215 | 0.083858468 | -11.94223123 | 7.13E-33 | 6.60E-31 | CA12 |
| 12.56259131 | -1.001393593 | 0.357721183 | -2.799369007 | 0.005120259 | 0.023217602 | SCAT8 |
| 5.458123772 | 1.000742686 | 0.361460365 | 2.768609736 | 0.005629602 | 0.024950381 | SEMA6C |

3. Discussion

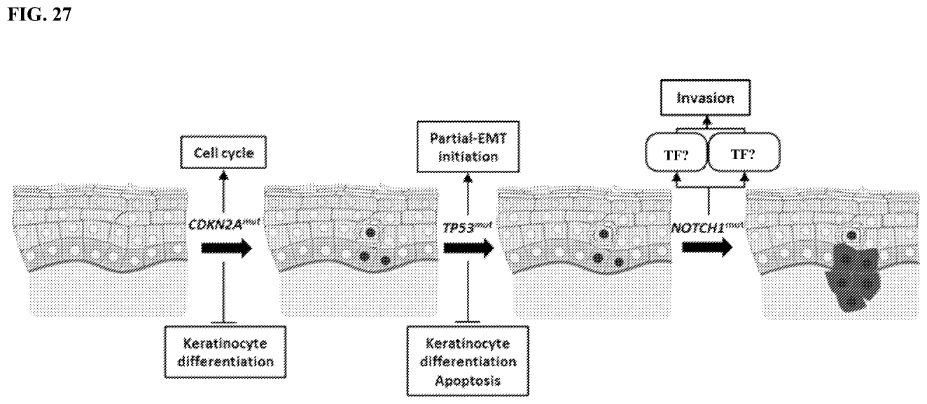

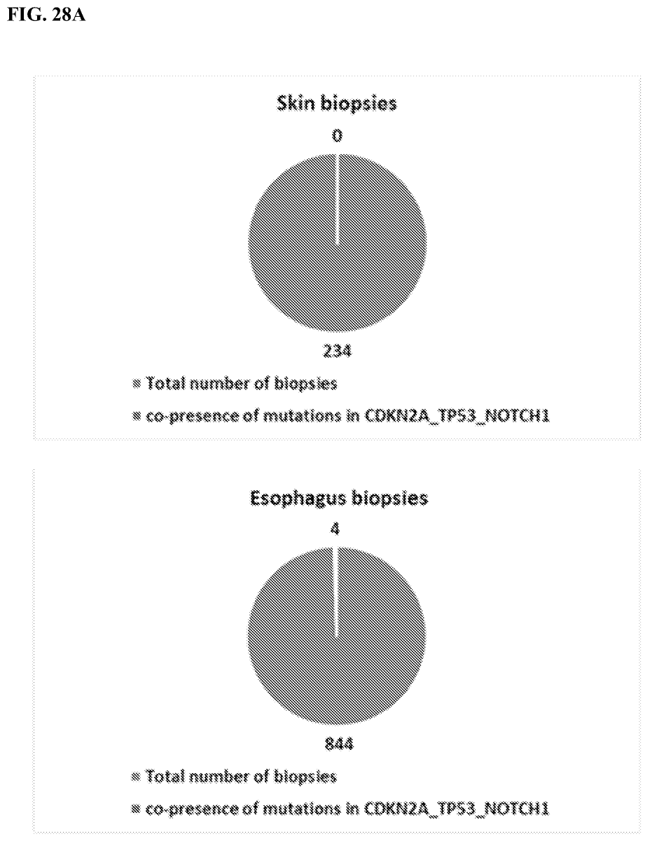

Our study has identified the minimal number of mutations at endogenous loci implicated in human SCC that is sufficient for malignant transformation. These findings are consistent with sequencing data from human tumors (7-12) and data from normal appearing squamous epithelium (17,18) suggesting that no single gene mutation is sufficient for SCC tumor initiation. We have been able to reproduce an invasive phenotype into human dermis by employing human primary keratinocytes and using a model that better recapitulates the complex 3D architecture of human tissue than simpler in vitro systems. This system allowed us to unveil phenotypic differences not detectable in our own 2D culture, highlighting the importance of developing and employing more realistic models in the study of cancer biology. Our results indicate that in order to initiate squamous neoplasia, early mutations in keratinocytes favor cell cycle progression while also impeding keratinocyte differentiation. A partial disruption of the keratinocyte differentiation program is normally seen in pre-malignant lesions, while more extensive disruption of differentiation is often detected in SCC tumors (23). Previous studies have reported conflicting data on the effect of CDKN2A and TP53 mutations on keratinocyte differentiation (24,25). Here we identify that simultaneous mutations in both genes are sufficient to disrupt keratinocyte differentiation using primary human keratinocytes. Other studies have reported significant associations between aneuploidy and gene mutation rates (26). TP53 and CDKN2A are among those genes with mutations that are selected through a series of genome rearrangements or duplication events in a significant manner; suggesting that these mutations are required for tumor maintenance. Our results support the hypothesis that these mutations are the main driving events in SCC initiation. Importantly, mutations in NOTCH1 are sufficient to induce an EMT-like transcriptional program in CDKN2A-TP53-deficient keratinocytes, which enables tumorigenic invasion of human dermis. These results coincide with previous reports supporting the importance of NOTCH1 as a tumor suppressor (27). In addition, recently published data suggests that multiple other mutations/genomic alterations favor the inactivation of NOTCH signaling in NOTCH receptor wild-type SCC cases (28). Supporting these findings, our results demonstrate that alteration of the NOTCH signaling pathway is crucial for the development of SCC in a subset of tumors (FIG. 27). Our results further suggest that initiation of an EMT-like process in keratinocytes accompanies invasive progression of SCC. These data are consistent with a recent single-cell RNA-seq analysis of HNSCC, which reported the activation of an EMT-like program at the leading edge of malignant lesions (29).

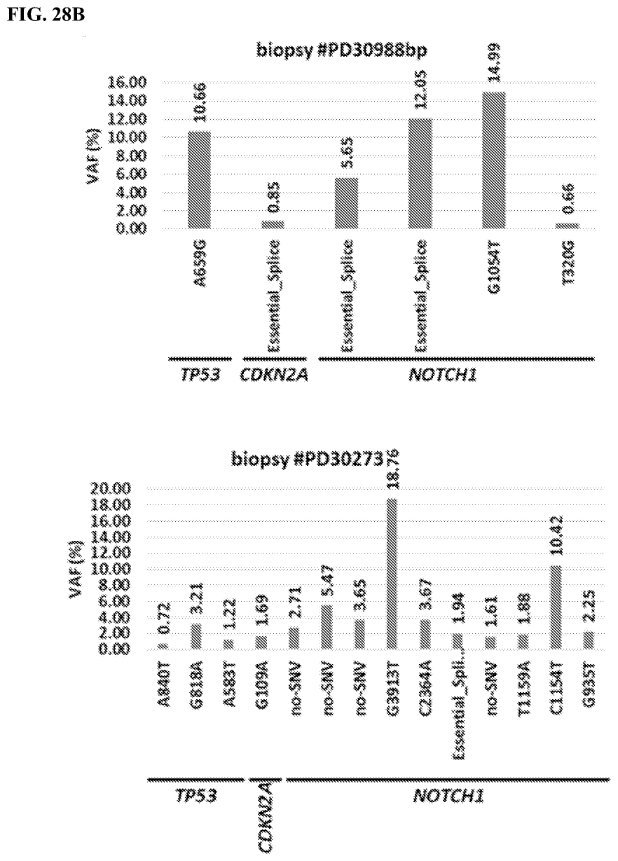

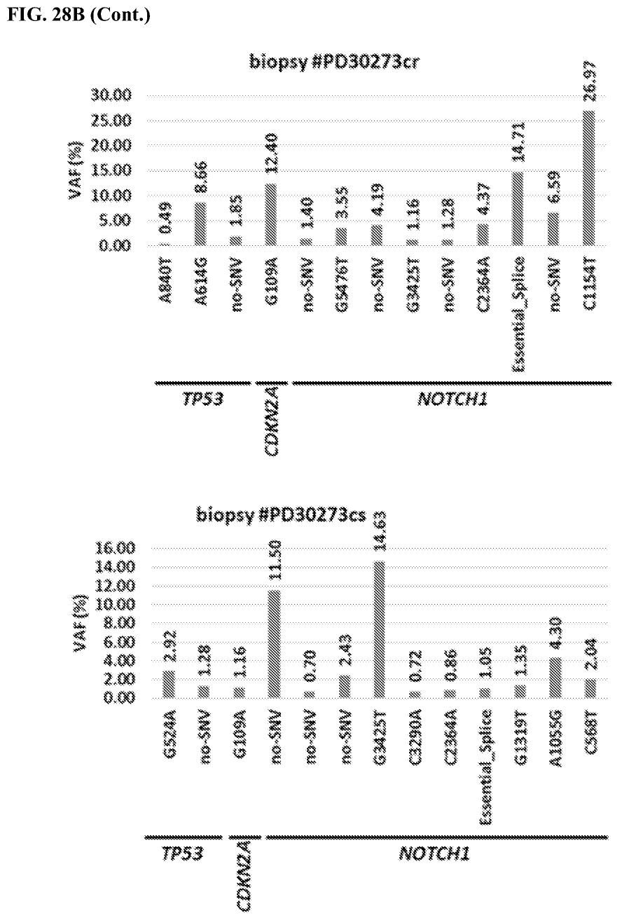

The epithelium serves as a robust physical barrier between the environment and the body. Collectively, prior studies have reported a higher mutational burden in HNSCC and other SCC types when compared to other malignancies (15) and the tendency for normal aged epithelia to accumulate mutations without resulting in SCC formation (17,18). These studies report that mutations in NOTCH1 are relatively frequent in aged epidermis, but mutations in TP53 and, specifically in CDKN2A, are rare and were not detected in healthy epithelium (FIG. 28). Altogether, this implies that a very specific combination of genetic alterations is necessary to trigger SCC initiation. It may be the case that stratified squamous epithelia, like the skin and oral mucosa, have evolved to be robust to the oncogenic effects of single mutations, as they bear a large burden of exposure to mutagenic stimuli through sun exposure and ingestion.

Co-occurring mutations in CDKN2A, TP53, and NOTCH1 are detected in 12% of HNSCC (TCGA Data). Interestingly, the loss of function of these three driver gene mutations is sufficient to transform keratinocytes into their malignant counterparts. Both our genome and RNA sequencing results argue against the necessity of other mutations (e.g. TERT mutation, PI3KCA activation) to achieve this state. It is certainly the case that there are other combinations of mutations sufficient to induce tumorigenesis. It is possible that a subset of driver events is more important in initiating tumorigenesis, whereas other events may confer a selective advantage only after transformation. Future work is required to better define the rules of mutational progression in SCC.

Finally, it is discovered that the introduction of mutations in NOTCH1 results in a transcriptomic shift, which leads to an EMT-like phenotype. These results suggest that the upregulation of certain transcription factors is essential for the completion of this process in this context.

4. Sequences

The sequences mentioned in the disclosure but not disclosed elsewhere can be found in the following tables.

pSpCas9 vector components

| | |
|---|---|
| pUC (Origin of replication): | SEQ ID |
| ttgagatcctttttttctgcgcgtaatctgctgcttgcaaacaaaaaaaccaccgctaccagcggtggtttgtttgccggatcaagagctacc | NO: 01 |
| aactctttttccgaaggtaactggcttcagcagagcgcagataccaaatactgtccttctagtgtagccgtagttaggccaccacttcaaga | |
| actctgtagcaccgcctacatacctcgctctgctaatcctgttaccagtggctgctgccagtggcgataagtcgtgtcttaccgggttggact | |
| caagacgatagttaccggataaggcgcagcggtcgggctgaacggggggttcgtgcacacagcccagcttggagcgaacgacctac | |
| accgaactgagatacctacagcgtgagctatgagaaagcgccacgcttcccgaagggagaaaggcggacaggtatccggtaagcgg | |
| cagggtcggaacaggagagcgcacgagggagcttccagggggaaacgcctggtatctttatagtcctgtcgggtttcgccacctctgac | |
| ttgagcgtcgatttttgtgatgctcgtcaggggggcggagcctatggaaa | |
| U6 promoter (Guide RNA promoter) | SEQ ID |
| gagggcctatttcccatgattccttcatatttgcatatacgatacaaggctgttagagagataattggaattaatttgactgtaaacacaaaga | NO: 02 |
| tattagtacaaaatacgtgacgtagaaagtaataatttcttgggtagtttgcagttttaaaattatgttttaaaatggactatcatatgcttac | |
| cgtaacttgaaagtatttcgatttcttggctttatatatcttgtggaaaggac | |
| CMV promoter (Cas9-GFP promoter) | SEQ ID |
| cgttacataacttacggtaaatggcccgcctggctgaccgcccaacgacccccgcccattgacgtcaatagtaacgccaatagggacttt | NO:03 |
| ccattgacgtcaatgggtggagtatttacggtaaactgcccacttggcagtacatcaagtgtatcatatgccaagtacgccccctattgacg | |
| tcaatgacggtaaatggcccgcctggcattgtgcccagtacatgaccttatgggactttcctacttggcagtacatctacgtattagtcatcg | |
| ctattaccatg | |
| Cas9 gene | SEQ ID |
| gacaagaagtacagcatcggcctggacatcggcaccaactctgtgggctgggccgtgatcaccgacgagtacaaggtgcccagcaag | NO: 04 |
| aaattcaaggtgctgggcaacaccgaccggcacagcatcaagaagaacctgatcggagccctgctgttcgacagcggcgaaacagcc | |
| gaggccacccggctgaagagaaccgccagaagaagatacaccagacggaagaaccggatctgctatctgcaagagatcttcagcaac | |
| gagatggccaaggtggacgacagcttcttccacagactggaagagtccttcctggtggaagaggataagaagcacgagcggcacccc | |
| atcttcggcaacatcgtggacgaggtggcctaccacgagaagtaccccaccatctaccacctgagaaagaaactggtggacagcaccg | |
| acaaggccgacctgcggctgatctatctggccctggcccacatgatcaagttccggggccacttcctgatcgagggcgacctgaacccc | |
| gacaacagcgacgtggacaagctgttcatccagctggtgcagacctacaaccagctgttcgaggaaaaccccatcaacgccagcggc | |
| gtggacgccaaggccatcctgtctgccagactgagcaagagcagacggctggaaaatctgatcgcccagctgcccggcgagaagaa | |
| gaatggcctgttcggaaacctgattgccctgagcctgggcctgacccccaacttcaagagcaacttcgacctggccgaggatgccaaac | |
| tgcagctgagcaaggacacctacgacgacgacctggacaacctgctggcccagatcggcgaccagtacgccgacctgtttctggccg | |
| ccaagaacctgtccgacgccatcctgctgagcgacatcctgagagtgaacaccgagatcaccaaggcccccctgagcgcctctatgat | |
| caagagatacgacgagcaccaccaggacctgaccctgctgaaagctctcgtgcggcagcagctgcctgagaagtacaaagagattttc | |
| ttcgaccagagcaagaacggctacgccggctacattgacggcggagccagccaggaagagttctacaagttcatcaagcccatcctgg | |
| aaaagatggacggcaccgaggaactgctcgtgaagctgaacagagaggacctgctgcggaagcagcggaccttcgacaacggcag | |
| catcccccaccagatccacctgggagagctgcacgccattctgcggcggcaggaagatttttacccattcctgaaggacaaccgggaaa | |
| agatcgagaagatcctgaccttccgcatcccctactacgtgggccctctggccaggggaaacagcagattcgcctggatgaccagaaa | |
| gagcgaggaaaccatcaccccctggaacttcgaggaagtggtggacaagggcgcttccgcccagagcttcatcgagcggatgaccaa | |
| cttcgataagaacctgcccaacgagaaggtgctgcccaagcacagcctgctgtacgagtacttcaccgtgtataacgagctgaccaaag | |
| tgaaatacgtgaccgagggaatgagaaagcccgccttcctgagcggcgagcagaaaaaggccatcgtggacctgctgttcaagacca | |
| accggaaagtgaccgtgaagcagctgaaagaggactacttcaagaaaatcgagtgcttcgactccgtggaaatctccggcgtggaaga | |
| tcggttcaacgcctccctgggcacataccacgatctgctgaaaattatcaaggacaaggacttcctggacaatgaggaaaacgaggaca | |
| ttctggaagatatcgtgctgaccctgacactgtttgaggacagagagatgatcgaggaacggctgaaaacctatgcccacctgttcgacg | |
| acaaagtgatgaagcagctgaagcggcggagatacaccggctggggcaggctgagccggaagctgatcaacggcatccgggacaa | |
| gcagtccggcaagacaatcctggatttcctgaagtccgacggcttcgccaacagaaacttcatgcagctgatccacgacgacagcctga | |
| cctttaaagaggacatccagaaagcccaggtgtccggccagggcgatagcctgcacgagcacattgccaatctggccggcagccccg | |
| ccattaagaagggcatcctgcagacagtgaaggtggtggacgagctcgtgaaagtgatgggccggcacaagcccgagaacatcgtga | |
| tcgaaatggccagagagaaccagaccacccagaagggacagaagaacagccgcgagagaatgaagcggatcgaagagggcatca | |
| aagagctgggcagccagatcctgaaagaacaccccgtggaaaacacccagctgcagaacgagaagctgtacctgtactacctgcaga | |
| atgggcgggatatgtacgtggaccaggaactggacatcaaccggctgtccgactacgatgtggaccatatcgtgcctcagagctttctga | |
| aggacgactccatcgacaacaaggtgctgaccagaagcgacaagaaccggggcaagagcgacaacgtgccctccgaagaggtcgt | |
| gaagaagatgaagaactactggcggcagctgctgaacgccaagctgattacccagagaaagttcgacaatctgaccaaggccgagag | |
| aggcggcctgagcgaactggataaggccggcttcatcaagagacagctggtggaaacccggcagatcacaaagcacgtggcacaga | |
| tcctggactcccggatgaacactaagtacgacgagaatgacaagctgatccgggaagtgaaagtgatcaccctgaagtccaagctggtg | |
| tccgatttccggaaggatttccagttttacaaagtgcgcgagatcaacaactaccaccacgcccacgacgcctacctgaacgccgtcgtg | |
| ggaaccgccctgatcaaaaagtaccctaagctggaaagcgagttcgtgtacggcgactacaaggtgtacgacgtgcggaagatgatcg | |
| ccaagagcgagcaggaaatcggcaaggctaccgccaagtacttcttctacagcaacatcatgaactttttcaagaccgagattaccctgg | |
| ccaacggcgagatccggaagcggcctctgatcgagacaaacggcgaaaccggggagatcgtgtgggataagggccgggattttgcc | |
| accgtgcggaaagtgctgagcatgccccaagtgaatatcgtgaaaaagaccgaggtgcagacaggcggcttcagcaaagagtctatcc | |
| tgcccaagaggaacagcgataagctgatcgccagaaagaaggactgggaccctaagaagtacggcggcttcgacagccccaccgtg | |
| gcctattctgtgctggtggtggccaaagtggaaaagggcaagtccaagaaactgaagagtgtgaaagagctgctggggatcaccatcat | |
| ggaaagaagcagcttcgagaagaatcccatcgactttctggaagccaagggctacaaagaagtgaaaaaggacctgatcatcaagctg | |
| cctaagtactccctgttcgagctggaaaacggccggaagagaatgctggcctctgccggcgaactgcagaagggaaacgaactggcc | |
| ctgccctccaaatatgtgaacttcctgtacctggccagccactatgagaagctgaagggctcccccgaggataatgagcagaaacagct | |
| gtttgtggaacagcacaagcactacctggacgagatcatcgagcagatcagcgagttctccaagagagtgatcctggccgacgctaatc | |
| tggacaaagtgctgtccgcctacaacaagcaccgggataagcccatcagagagcaggccgagaatatcatccacctgtttaccctgacc | |
| aatctgggagcccctgccgccttcaagtactttgacaccaccatcgaccggaagaggtacaccagcaccaaagaggtgctggacgcca | |
| ccctgatccaccagagcatcaccggcctgtacgagacacggatcgacctgtctcagctgggaggcgac | |
| GFP gene | SEQ ID |
| gtgagcaagggcgaggagctgttcaccggggtggtgcccatcctggtcgagctggacggcgacgtaaacggccacaagttcagcgtg | NO: 05 |
| tccggcgagggcgagggcgatgccacctacggcaagctgaccctgaagttcatctgcaccaccggcaagctgcccgtgccctggccc | |
| accctcgtgaccaccctgacctacggcgtgcagtgcttcagccgctaccccgaccacatgaagcagcacgacttcttcaagtccgccat | |
| gcccgaaggctacgtccaggagcgcaccatcttcttcaaggacgacggcaactacaagacccgcgccgaggtgaagttcgagggcg | |
| acaccctggtgaaccgcatcgagctgaagggcatcgacttcaaggaggacggcaacatcctggggcacaagctggagtacaactaca | |
| acagccacaacgtctatatcatggccgacaagcagaagaacggcatcaaggtgaacttcaagatccgccacaacatcgaggacggca | |

gcgtgcagctcgccgaccactaccagcagaacacccccatcggcgacggccccgtgctgctgcccgacaaccactacctgagcaccc
agtccgccctgagcaaagaccccaacgagaagcgcgatcacatggtcctgctggagttcgtgaccgccgccgggatcactctcggcat
ggacgagctgtacaag

| | |
|---|---|
| Ampicillin promoter | SEQ ID |
| cgcggaacccctatttgtttatttttctaaatacattcaaatatgtatccgctcatgagacaataaccctgataaatgcttcaataatattga | NO: 06 |
| aaaaggaagagt | |
| Ampicillin (Bacteria selection marker) | SEQ ID |
| atgagtattcaacatttccgtgtcgcccttattcccttttttgcggcattttgccttcctgtttttgctcacccagaaacgctggtgaaagt | NO: 07 |
| aaaagatgctgaagatcagttgggtgcacgagtgggttacatcgaactggatctcaacagcggtaagatccttgagagttttcgccccgaagaa | |
| cgttttccaatgatgagcacttttaaagttctgctatgtggcgcggtattatcccgtattgacgccgggcaagagcaactcggtcgccgcata | |
| cactattctcagaatgacttggttgagtactcaccagtcacagaaaagcatcttacggatggcatgacagtaagagaattatgcagtgctgc | |
| cataaccatgagtgataacactgcggccaacttacttctgacaacgatcggaggaccgaaggagctaaccgcttttttgcacaacatggg | |
| ggatcatgtaactcgccttgatcgttgggaaccggagctgaatgaagccataccaaacgacgagcgtgacaccacgatgcctgtagcaa | |
| tggcaacaacgttgcgcaaactattaactggcgaactacttactctagcttcccggcaacaattaatagactggatggaggcggataaagt | |
| tgcaggaccacttctgcgctcggcccttccggctggctggtttattgctgataaatctggagccggtgagcgtggaagccgcggtatcatt | |
| gcagcactggggccagatggtaagccctcccgtatcgtagttatctacacgacggggagtcaggcaactatggatgaacgaaatagac | |
| agatcgctgagataggtgcctcactgattaagcattggtaa | |
| Guide RNA scaffold | SEQ ID |
| gttttagagctagaaatagcaagttaaaataaggctagtccgttatcaacttgaaaaagtggcaccgagtcggtgc | NO: 08 |
| Full pSpCas9-2A-BB-GFP vector sequence | SEQ ID |
| gagggcctatttcccatgattccttcatatttgcatatacgatacaaggctgttagagagataattggaattaatttgactgtaaacacaaag | NO: 09 |
| atattagtacaaaatacgtgacgtagaaagtaataatttcttgggtagtttgcagttttaaaattatgttttaaaatggactatcatatgctt | |
| accgtaacttgaaagtatttcgatttcttggctttatatatcttgtggaaaggacgaaacaccgggtcttcgagaagacctgttttagagcta | |
| gaaatagcaagttaaaataaggctagtccgttatcaacttgaaaaagtggcaccgagtcggtgcttttttgttttagagctagaaatagcaag | |
| ttaaaataaggctagtccgtttttagcgcgtgcgccaattctgcagacaaatggctctagaggtacccgttacataacttacggtaaatggcc | |
| cgcctggctgaccgcccaacgacccccgcccattgacgtcaatagtaacgccaatagggactttccattgacgtcaatgggtggagtatttac | |
| ggtaaactgcccacttggcagtacatcaagtgtatcatatgccaagtacgccccctattgacgtcaatgacggtaaatggcccgcctggcattg | |
| tgcccagtacatgaccttatgggactttcctacttggcagtacatctacgtattagtcatcgctattaccatggtcgaggtgagccccacgttc | |
| tgcttcactctccccatctcccccccctccccacccccaattttgtatttatttattttttaattattttgtgcagcgatgggggcgggggggg | |
| ggggggggcgcgcgccaggcggggcggggcggggcgaggggcggggcggggcgaggcggagaggtgcggcggcagccaatc | |
| agagcggcgcgctccgaaagtttccttttatggcgaggcggcggcggcggcggccctataaaaagcgaagcgcgcggcgggcggg | |
| agtcgctgcgacgctgccttcgccccgtgccccgctccgccgccgcctcgcgccgcccgccccggctctgactgaccgcgttactccc | |
| acaggtgagcgggcgggacggcccttctcctccgggctgtaattagctgagcaagaggtaagggtttaagggatggttggttggtggg | |
| gtattaatgtttaattacctggagcacctgcctgaaatcactttttttcaggttggaccggtgccaccatggactataaggaccacgacggag | |
| actacaaggatcatgatattgattacaaagacgatgacgataagatggccccaaagaagaagcggaaggtcggtatccacggagtccc | |
| agcagccgacaagaagtacagcatcggcctggacatcggcaccaactctgtgggctgggccgtgatcaccgacgagtacaaggtgcc | |
| cagcaagaaattcaaggtgctgggcaacaccgaccggcacagcatcaagaagaacctgatcggagccctgctgttcgacagcggcga | |
| aacagccgaggccacccggctgaagagaaccgccagaagaagatacaccagacggaagaaccggatctgctatctgcaagagatctt | |
| cagcaacgagatggccaaggtggacgacagcttcttccacagactggaagagtccttcctggtggaagaggataagaagcacgagcg | |
| gcaccccatcttcggcaacatcgtggacgaggtggcctaccacgagaagtaccccaccatctaccacctgagaaagaaactggtggac | |
| agcaccgacaaggccgacctgcggctgatctatctggccctggcccacatgatcaagttccggggccacttcctgatcgagggcgacct | |
| gaaccccgacaacagcgacgtggacaagctgttcatccagctggtgcagacctacaaccagctgttcgaggaaaaccccatcaacgcc | |
| agcggcgtggacgccaaggccatcctgtctgccagactgagcaagagcagacggctggaaaatctgatcgcccagctgcccggcga | |
| gaagaagaatggcctgttcggaaacctgattgccctgagcctgggcctgacccccaacttcaagagcaacttcgacctggccgaggatg | |
| ccaaactgcagctgagcaaggacacctacgacgacgacctggacaacctgctggcccagatcggcgaccagtacgccgacctgtttct | |
| ggccgccaagaacctgtccgacgccatcctgctgagcgacatcctgagagtgaacaccgagatcaccaaggcccccctgagcgcctc | |
| tatgatcaagagatacgacgagcaccaccaggacctgaccctgctgaaagctctcgtgcggcagcagctgcctgagaagtacaaagag | |
| attttcttcgaccagagcaagaacggctacgccggctacattgacggcggagccagccaggaagagttctacaagttcatcaagcccat | |
| cctggaaaagatggacggcaccgaggaactgctcgtgaagctgaacagagaggacctgctgcggaagcagcggaccttcgacaacg | |
| gcagcatcccccaccagatccacctgggagagctgcacgccattctgcggcggcaggaagatttttacccattcctgaaggacaaccg | |
| ggaaaagatcgagaagatcctgaccttccgcatcccctactacgtgggccctctggccaggggaaacagcagattcgcctggatgacc | |
| agaaagagcgaggaaaccatcaccccctggaacttcgaggaagtggtggacaagggcgcttccgcccagagcttcatcgagcggatg | |
| accaacttcgataagaacctgcccaacgagaaggtgctgcccaagcacagcctgctgtacgagtacttcaccgtgtataacgagctgac | |
| caaagtgaaatacgtgaccgagggaatgagaaagcccgccttcctgagcggcgagcagaaaaaggccatcgtggacctgctgttcaa | |
| gaccaaccggaaagtgaccgtgaagcagctgaaagaggactacttcaagaaaatcgagtgcttcgactccgtggaaatctccggcgtg | |
| gaagatcggttcaacgcctccctgggcacataccacgatctgctgaaaattatcaaggacaaggacttcctggacaatgaggaaaacga | |
| ggacattctggaagatatcgtgctgaccctgacactgtttgaggacagagagatgatcgaggaacggctgaaaacctatgcccacctgtt | |
| cgacgacaaagtgatgaagcagctgaagcggcggagatacaccggctggggcaggctgagccggaagctgatcaacggcatccgg | |
| gacaagcagtccggcaagacaatcctggatttcctgaagtccgacggcttcgccaacagaaacttcatgcagctgatccacgacgacag | |
| cctgacctttaaagaggacatccagaaagcccaggtgtccggccagggcgatagcctgcacgagcacattgccaatctggccggcag | |
| ccccgccattaagaagggcatcctgcagacagtgaaggtggtggacgagctcgtgaaagtgatgggccggcacaagcccgagaacat | |
| cgtgatcgaaatggccagagagaaccagaccacccagaagggacagaagaacagccgcgagagaatgaagcggatcgaagaggg | |
| catcaaagagctgggcagccagatcctgaaagaacaccccgtggaaaacacccagctgcagaacgagaagctgtacctgtactacctg | |
| cagaatgggcgggatatgtacgtggaccaggaactggacatcaaccggctgtccgactacgatgtggaccatatcgtgcctcagagctt | |
| tctgaaggacgactccatcgacaacaaggtgctgaccagaagcgacaagaaccggggcaagagcgacaacgtgccctccgaagag | |
| gtcgtgaagaagatgaagaactactggcggcagctgctgaacgccaagctgattacccagagaaagttcgacaatctgaccaaggccg | |
| agagaggcggcctgagcgaactggataaggccggcttcatcaagagacagctggtggaaacccggcagatcacaaagcacgtggca | |
| cagatcctggactcccggatgaacactaagtacgacgagaatgacaagctgatccgggaagtgaaagtgatcaccctgaagtccaagc | |
| tggtgtccgatttccggaaggatttccagttttacaaagtgcgcgagatcaacaactaccaccacgcccacgacgcctacctgaacgccg | |
| tcgtgggaaccgccctgatcaaaaagtaccctaagctggaaagcgagttcgtgtacggcgactacaaggtgtacgacgtgcggaagat | |
| gatcgccaagagcgagcaggaaatcggcaaggctaccgccaagtacttcttctacagcaacatcatgaactttttcaagaccgagattac | |
| cctggccaacggcgagatccggaagcggcctctgatcgagacaaacggcgaaaccggggagatcgtgtgggataagggccgggatt | |
| ttgccaccgtgcggaaagtgctgagcatgccccaagtgaatatcgtgaaaaagaccgaggtgcagacaggcggcttcagcaaagagtc | |
| tatcctgcccaagaggaacagcgataagctgatcgccagaaagaaggactgggaccctaagaagtacggcggcttcgacagccccac | |
| cgtggcctattctgtgctggtggtggccaaagtggaaaagggcaagtccaagaaactgaagagtgtgaaagagctgctggggatcacc | |
| atcatggaaagaagcagcttcgagaagaatcccatcgactttctggaagccaagggctacaaagaagtgaaaaaggacctgatcatcaa | |
| gctgcctaagtactccctgttcgagctggaaaacggccggaagagaatgctggcctctgccggcgaactgcagaagggaaacgaact | |
| ggccctgccctccaaatatgtgaacttcctgtacctggccagccactatgagaagctgaagggctcccccgaggataatgagcagaaac | |
| agctgtttgtggaacagcacaagcactacctggacgagatcatcgagcagatcagcgagttctccaagagagtgatcctggccgacgct | |
| aatctggacaaagtgctgtccgcctacaacaagcaccgggataagcccatcagagagcaggccgagaatatcatccacctgtttaccct | |
| gaccaatctgggagcccctgccgccttcaagtactttgacaccaccatcgaccggaagaggtacaccagcaccaaagaggtgctggac | |
| gccaccctgatccaccagagcatcaccggcctgtacgagacacggatcgacctgtctcagctgggaggcgacaaaaggccggcggc | |
| cacgaaaaaggccggccaggcaaaaaagaaaaaggaattcggcagtggagagggcagaggaagtctgctaacatgcggtgacgtc | |
| gaggagaatcctggcccagtgagcaagggcgaggagctgttcaccggggtggtgcccatcctggtcgagctggacggcgacgtaaa | |
| cggccacaagttcagcgtgtccggcgagggcgagggcgatgccacctacggcaagctgaccctgaagttcatctgcaccaccggcaa | |
| gctgcccgtgccctggcccaccctcgtgaccaccctgacctacggcgtgcagtgcttcagccgctaccccgaccacatgaagcagcac | |
| gacttcttcaagtccgccatgcccgaaggctacgtccaggagcgcaccatcttcttcaaggacgacggcaactacaagacccgcgccg | |
| aggtgaagttcgagggcgacaccctggtgaaccgcatcgagctgaagggcatcgacttcaaggaggacggcaacatcctggggcac | |
| aagctggagtacaactacaacagccacaacgtctatatcatggccgacaagcagaagaacggcatcaaggtgaacttcaagatccgcc | |
| acaacatcgaggacggcagcgtgcagctcgccgaccactaccagcagaacacccccatcggcgacggccccgtgctgctgcccgac | |
| aaccactacctgagcacccagtccgccctgagcaaagaccccaacgagaagcgcgatcacatggtcctgctggagttcgtgaccgcc | |
| gccgggatcactctcggcatggacgagctgtacaaggaattctaactagagctcgctgatcagcctcgactgtgccttctagttgccagcc | |
| atctgttgtttgcccctcccccgtgccttccttgaccctggaaggtgccactcccactgtcctttcctaataaaatgaggaaattgcatcgcat | |
| tgtctgagtaggtgtcattctattctggggggtggggtggggcaggacagcaagggggaggattgggaagagaatagcaggcatgctg | |
| gggagcggccgcaggaacccctagtgatggagttggccactccctctctgcgcgctcgctcgctcactgaggccgggcgaccaaagg | |
| tcgcccgacgcccgggctttgcccgggcggcctcagtgagcgagcgagcgcgcagctgcctgcaggggcgcctgatgcggtattttct | |
| ccttacgcatctgtgcggtatttcacaccgcatacgtcaaagcaaccatagtacgcgccctgtagcggcgcattaagcgcggcgggtgtg | |
| gtggttacgcgcagcgtgaccgctacacttgccagcgccctagcgcccgctcctttcgctttcttcccttcctttctcgccacgttcgccgg | |
| ctttccccgtcaagctctaaatcgggggctccctttagggttccgatttagtgctttacggcacctcgaccccaaaaaacttgatttgggtgat | |
| ggttcacgtagtgggccatcgccctgatagacggtttttcgccctttgacgttggagtccacgttctttaatagtggactcttgttccaaactg | |
| gaacaacactcaaccctatctcgggctattcttttgatttataagggattttgccgatttcggcctattggttaaaaaatgagctgatttaaca | |
| aaaatttaacgcgaattttaacaaaatattaacgtttacaattttatggtgcactctcagtacaatctgctctgatgccgcatagttaagccag | |
| ccccgacacccgccaacacccgctgacgcgccctgacgggcttgtctgctcccggcatccgcttacagacaagctgtgaccgtctccggga | |
| gctgcatgtgtcagaggttttcaccgtcatcaccgaaacgcgcgagacgaaagggcctcgtgatacgcctatttttataggttaatgtcatg | |
| ataataatggtttcttagacgtcaggtggcacttttcggggaaatgtgcgcggaacccctatttgtttatttttctaaatacattcaaatatgt | |
| atccgctcatgagacaataaccctgataaatgcttcaataatattgaaaaaggaagagtatgagtattcaacatttccgtgtcgcccttattcc | |
| tttgcggcattttgccttcctgtttttgctcacccagaaacgctggtgaaagtaaaagatgctgaagatcagttgggtgcacgagtgggttac | |
| atcgaactggatctcaacagcctttggtaagatccttgagagttttcgccccgaagaacgttttccaatgatgagcacttttaaagttctgcta | |
| tgtggcgcggtattatcccgtattgacgccgggcaagagcaactcggtcgccgcatacactattctcagaatgacttggttgagtactcaccag | |
| tcacagaaaagcatcttacggatggcatgacagtaagagaattatgcagtgctgccataaccatgagtgataacactgcggccaacttactt | |
| ctgacaacgatcggaggaccgaaggagctaaccgcttttttgcacaacatgggggatcatgtaactcgccttgatcgttgggaaccgga | |
| gctgaatgaagccataccaaacgacgagcgtgacaccacgatgcctgtagcaatggcaacaacgttgcgcaaactattaactggcgaa | |
| ctacttactctagcttcccggcaacaattaatagactggatggaggcggataaagttgcaggaccacttctgcgctcggcccttccggctg | |
| gctggtttattgctgataaatctggagccggtgagcgtggaagccgcggtatcattgcagcactggggccagatggtaagccctcccgta | |
| tcgtagttatctacacgacggggagtcaggcaactatggatgaacgaaatagacagatcgctgagataggtgcctcactgattaagcatt | |
| ggtaactgtcagaccaagtttactcatatatactttagattgatttaaaacttcatttttaatttaaaaggatctaggtgaagatcctttttg | |
| ataatctcatgaccaaaatcccttaacgtgagttttcgttccactgagcgtcagaccccgtagaaaagatcaaaggatcttcttgagatccttt | |
| ttttctgcgcgtaatctgctgcttgcaaacaaaaaaaccaccgctaccagcggtggtttgtttgccggatcaagagctaccaactctttttcc | |
| gaaggtaactggcttcagcagagcgcagataccaaatactgtccttctagtgtagccgtagttaggccaccacttcaagaactctgtagcaccg | |
| cctacatacctcgctctgctaatcctgttaccagtggctgctgccagtggcgataagtcgtgtcttaccgggttggactcaagacgatagttac | |
| cggataaggcgcagcggtcgggctgaacggggggttcgtgcacacagcccagcttggagcgaacgacctacaccgaactgagatacc | |
| tacagcgtgagctatgagaaagcgccacgcttcccgaagggagaaaggcggacaggtatccggtaagcggcagggtcggaacagg | |
| agagcgcacgagggagcttccagggggaaacgcctggtatctttatagtcctgtcgggtttcgccacctctgacttgagcgtcgatttttgt | |
| gatgctcgtcaggggggcggagcctatggaaaaacgccagcaacgcggcctttttacggttcctggccttttgctggccttttgctcacat | |
| gt | |

Selection markers (including fluorescent gene reporters, antibiotic resistance genes and surface
proteins)

| | |
|---|---|
| GFP | SEQ ID |
| | NO: 317 |
| Tdtomato | SEQ ID |
| atggtgagcaagggcgaggaggtcatcaaagagttcatgcgcttcaaggtgcgcatggagggctccatgaacggccacgagttcga | NO: 10 |
| gatcgagggcgagggcgagggccgcccctacgagggcacccagaccgccaagctgaaggtgaccaagggcggccccctgccc | |
| ttcgcctgggacatcctgtccccccagttcatgtacggctccaaggcgtacgtgaagcaccccgccgacatccccgattacaagaag | |
| ctgtccttccccgagggcttcaagtgggagcgcgtgatgaacttcgaggacggcggtctggtgaccgtgacccaggactcctccctg | |
| caggacggcacgctgatctacaaggtgaagatgcgcggcaccaacttcccccccgacggccccgtaatgcagaagaagaccatgg | |
| gctgggaggcctccaccgagcgcctgtacccccgcgacggcgtgctgaagggcgagatccaccaggccctgaagctgaaggac | |
| ggcggccactacctggtggagttcaagaccatctacatggccaagaagcccgtgcaactgcccggctactactacgtggacaccaa | |
| gctggacatcacctcccacaacgaggactacaccatcgtggaacagtacgagcgctccgagggccgccaccacctgttcctgggg | |
| catggcaccggcagcaccggcagcggcagctccggcaccgcctcctccgaggacaacaacatggccgtcatcaaagagttcatgc | |
| gcttcaaggtgcgcatggagggctccatgaacggccacgagttcgagatcgagggcgagggcgagggccgcccctacgagggc | |
| acccagaccgccaagctgaaggtgaccaagggcggccccctgcccttcgcctgggacatcctgtccccccagttcatgtacggctc | |
| caaggcgtacgtgaagcaccccgccgacatccccgattacaagaagctgtccttccccgagggcttcaagtgggagcgcgtgatga | |
| acttcgaggacggcggtctggtgaccgtgacccaggactcctccctgcaggacggcacgctgatctacaaggtgaagatgcgcgg | |
| caccaacttcccccccgacggccccgtaatgcagaagaagaccatgggctgggaggcctccaccgagcgcctgtacccccgcga | |
| cggcgtgctgaagggcgagatccaccaggccctgaagctgaaggacggcggccactacctggtggagttcaagaccatctacatg | |
| gccaagaagcccgtgcaactgcccggctactactacgtggacaccaagctggacatcacctcccacaacgaggactacaccatcgt | |
| ggaacagtacgagcgctccgagggccgccaccacctgttcctgtacggcatggacgagctgtacaagtaa | |
| EBFP2 | SEQ ID |
| MVSKGEELFTGVVPILVELDGDVNGHKFSVRGEGEGDATNGKLTLKFICTTG | NO: 11 |
| KLPVPWPTLVTTLSHGVQCFARYPDHMKQHDFFKSAMPEGYVQERTIFFKD | |
| DGTYKTRAEVKFEGDTLVNRIELKGVDFKEDGNILGHKLEYNFNSHNIYIMA | |
| VKQKNGIKVNFKIRHNVEDGSVQLADHYQQNTPIGDGPVLLPDSHYLSTQSV | |
| LSKDPNEKRDHMVLLEFRTAAGITLGMDELYK | |
| mKate2 | SEQ ID |
| atggtgagcgagctgattaaggagaacatgcacatgaagctgtacatggagggcaccgtgaacaaccaccacttcaagtgcacatcc | NO: 12 |
| gagggcgaaggcaagccctacgagggcacccagaccatgagaatcaaggcggtcgagggcggccctctccccttcgccttcgac | |
| atcctggctaccagcttcatgtacggcagcaaaaccttcatcaaccacacccagggcatccccgacttctttaagcagtccttccctga | |
| gggcttcacatgggagagagtcaccacatacgaagacgggggcgtgctgaccgctacccaggacaccagcctccaggacggctg | |
| cctcatctacaacgtcaagatcagaggggtgaacttcccatccaacggccctgtgatgcagaagaaaacactcggctgggaggcctc | |
| caccgagaccctgtaccccgctgacggcggcctggaaggcagagccgacatggccctgaagctcgtgggcgggggccacctgat | |
| ctgcaacttgaagaccacatacagatccaagaaacccgctaagaacctcaagatgcccggcgtctactatgtggacagaagactgga | |
| aagaatcaaggaggccgacaaagagacctacgtcgagcagcacgaggtggctgtggccagatactgcgacctccctagcaaactg | |
| gggcac | |
| mKO | SEQ ID |
| atggtgagtgtgattaaaccagagatgaagatgaggtactacatggacggctccgtcaatgggcatgagttcacaattgaaggtgaag | NO: 13 |
| gcacaggcagaccttacgagggacatcaagagatgacactacgcgtcacaatggccaagggcgggccaatgcctttcgcgtttgac | |
| ttagtgtcacacgtgttctgttacggccacagaccttttactaaatatccagaagagataccagactatttcaaacaagcatttcctg | |
| aaggcctgtcatgggaaaggtcgttggagttcgaagatggtgggtccgcttcagtcagtgcgcatataagccttagaggaaacacctt | |
| ctaccacaaatccaaatttactggggttaactttcctgccgatggtcctatcatgcaaaaccaaagtgttgattgggagccatcaacc | |
| gagaaaattactgccagcgacggagttctgaagggtgatgttacgatgtacctaaaacttgaaggaggcggcaatcacaaatgccaat | |
| tcaagactacttacaaggcggcaaaaaagattcttaaaatgccaggaagccattacatcagccatcgcctcgtcaggaaaaccgaagg | |
| caacattactgagctggtagaagatgcagtagctcattcc | |
| Puromycin | SEQ ID |
| atgaccgagtacaagcccacggtgcgcctcgccacccgcgacgacgtccccagggccgtacgcaccctcgccgccgcgttcgcc | NO: 14 |
| gactaccccgccacgcgccacaccgtcgatccggaccgccacatcgagcgggtcaccgagctgcaagaactcttcctcacgcgcg | |
| tcgggctcgacatcggcaaggtgtgggtcgcggacgacggcgccgcggtggcggtctggaccacgccggagagcgtcgaagcg | |
| ggggcggtgttcgccgagatcggcccgcgcatggccgagttgagcggttcccggctggccgcgcagcaacagatggaaggcctc | |
| ctggcgccgcaccggcccaaggagcccgcgtggttcctggccaccgtcggcgtctcgcccgaccaccagggcaagggtctgggc | |
| agcgccgtcgtgctccccggagtggaggcggccgagcgcgccggggtgcccgccttcctggagacctccgcgccccgcaacctc | |
| cccttctacgagcggctcggcttcaccgtcaccgccgacgtcgaggtgcccgaaggaccgcgcacctggtgcatgacccgcaagc | |
| ccggtgcctga | |
| DeltaLNGFR + SBP (Streptavidin binding peptide) | SEQ ID |
| atggacgaaaagaccacaggatggcgaggaggacacgtggtcgagggactggcaggagagctggaacagctgcgggctagact | NO: 15 |
| ggaacaccatcctcagggacagcgagagccaggaagtggagcgatcgcgaaggaagcctgccccacagggctgtacactcattct | |
| ggagaatgctgtaaagcttgtaacctgggagagggagtggcacagccatgcggagccaatcagactgtgtgcgagccttgtctgga | |
| ctccgtcacattctctgatgtggtcagtgccacagaaccttgcaagccatgtactgagtgcgtgggcctgcagtctatgagtgctcct | |
| tgtgtggaggctgacgatgcagtctgccggtgtgcatacggatactatcaggacgagactaccggcagatgtgaagcttgcagggtgt | |
| gtgaggcaggctcagggctggtctttagctgccaggataaacagaacaccgtgtgcgaggaatgtcctgacgggacatatagcgatga | |
| ggccaatcacgtggacccctgcctgccttgtactgtgtgcgaggataccgaaaggcagctgcgcgaatgtaccagatgggcagacg | |
| ccgagtgcgaggaaatcccagggcgatggattactcggtccaccccccctgaaggatcagacagcaccgcaccatctacacagga | |
| gccagaagcaccaccagagcaggatctgatcgcctccaccgtggctggcgtggtcacaactgtcatggggagctcccagccagtg | |
| gtcacccggggcaccacagataacctgattcccgtgtattgctccatcctggcagccgtcgtcgtcggactggtggcatacatcgcctt | |
| caagcggtga | |

Wild type genomic sequences genes:

| | |
|---|---|
| TP53 | SEQ ID |
| ctcattctccaggcttcagacctgtctccctcattcaaaaaatatttattatcgagctcttacttgctacccagcactgatataggcactcag | NO: 307 |
| gaatacaacaatgaataagatagtagaaaaattctatatcctcataaggcttacgtttccatgtactgaaagcaatgaacaaataaatctta | |
| tcagagtgataagggttgtgaaggagattaaataagatggtgtgatataaagtatctgggagaaaacgttagggtgtgatattacggaa | |
| agccttcctaaaaaatgacattttaactgatgagaagaaaggatccagctgagagcaaacgcaaaagctttcttccttccacccttcatat | |
| ttgacacaatgcaggattcctccaaaatgatttccaccaattctgccctcacagctctggcttgcagaattttccaccccaaaatgttagta | |
| tctacggcaccaggtcggcgagaatcctgactctgcaccctcctccccaactccatttcctttgcttcctccggcaggcggattacttgc | |
| ccttacttgtcatggcgactgtccagctttgtgccaggagcctcgcaggggttgatgggattggggttttcccctcccatgtgctcaaga | |
| ctggcgctaaaagttttgagcttctcaaaagtctagagccaccgtccagggagcaggtagctgctgggctccggggacactttgcgtt | |
| cgggctgggagcgtgctttccacgacggtgacacgcttccctggattgggtaagctcctgactgaacttgatgagtcctctctgagtca | |
| cgggctctcggctccgtgtattttcagctcgggaaaatcgctggggctgggggtggggcagtggggacttagcgagtttgggggtga | |
| gtgggatggaagcttggctagagggatcatcataggagttgcattgttgggagacctgggtgtagatgatggggatgttaggaccatc | |
| cgaactcaaagttgaacgcctaggcagaggagtggagctttggggaaccttgagccggcctaaagcgtacttctttgcacatccaccc | |
| ggtgctgggcgtagggaatccctgaaataaaagatgcacaaagcattgaggtctgagacttttggatctcgaaacattgagaactcata | |
| gctgtatattttagagcccatggcatcctagtgaaaactggggctccattccgaaatgatcatttgggggtgatccggggagcccaagc | |
| tgctaaggtcccacaacttccggacctttgtccttcctggagcgatctttccaggcagcccccggctccgctagatggagaaaatccaa | |
| ttgaaggctgtcagtcgtggaagtgagaagtgctaaaccaggggtttgcccgccaggccgaggaggaccgtcgcaatctgagagg | |
| cccggcagccctgttattgtttggctccacatttacatttctgcctcttgcagcagcatttccggtttctttttgccggagcagctcactattc | |
| acccgatgagaggggaggagagagagagaaaatgtcctttaggccggttcctcttacttggcagagggaggctgctattctccgcct | |
| gcatttctttttctggattacttagttatggcctttgcaaaggcaggggtatttgttttgatgcaaacctcaatccctccccttctttgaatg | |
| gtgtgccccaccccgcgggtcgcctgcaacctaggcggacgctaccatggcgtgagacagggagggaaagaagtgtgcagaaggca | |
| agcccggaggtattttcaagaatgagtatatctcatcttcccggaggaaaaaaaaaaagaatgggtacgtctgagaatcaaattttgaaa | |
| gagtgcaatgatgggtcgtttgataatttgtcggaaaaacaatctacctgttatctagctttgggctaggccattccagttccagacgcag | |
| gctgaacgtcgtgaagcggaaggggcgggcccgcaggcgtccgtgtggtcctccgtgcagccctccggcccgagccggttcttcc | |
| tggtaggaggcggaactcgaattcatttctcccgctgccccatctcttagctcgcggttgtttcattccgcagtttcttcccatgcacctgc | |
| cgcgtaccggccactttgtgccgtacttacgtcatctttttcctaaatcgaggtggcatttacacacagcgccagtgcacacagcaagtg | |
| cacaggaagatgagttttggcccctaaccgctccgtgatgcctaccaagtcacagacccttttcatcgtcccagaaacgtttcatcacgt | |
| ctcttcccagtcgattcccgaccccacctttattttgatctccataaccattttgcctgttggagaacttcatatagaatggaatcaggctgg | |
| gcgctgtggctcacgcctgcactttgggaggccgaggcgggcggattacttgaggataggagttccagaccagcgtggccaacgtg | |
| gtgaatccccgtctctactaaaaaatacaaaaattagctgggcgtggtgggtgcctgtaatcccagctattcgggagggtgaggcagg | |
| agaatcgcttgaacccgggaggcagaggttgcagtgagccaagatcgtgccactacactccagcctgggcgacaagaacgaaact | |
| ccgtctcaaaaaaaaggggggaatcatacattatgtgctcatttttgtcgggcttctgtccttcaatgtactgtctgacattcgttcatgttgt | |
| atatatcagtattttgctccttttcatttagtatagtccatcgattgtatatccgtccttttgatggccttttgagttgtttcccatttgcgg | |
| ttatgaaataaagctgctataaacattcttgtacaattctttttgtgatcatatgttttcgtgtttcttggagaaatacttaggaggggaatt | |
| gcgagtttggaagtaaaaagtagctgtattttgaactttttcagaagctctgagttttccagagcggttgtaccattttacactccaactagc | |
| aaggtatgggagttattatggttgtgccacagccttccggacattaggtattgtcagtctttctaatgtggtatatccttgtggttgtaattta | |
| cagttctctattgactaaggatgttcagcattttttcatgtgcctattggccattcgtattttgtttgtaaagtagctcttcgagtcttttacc | |
| tgttattttggttttttgtttgtttttattgttcagttgtgggactgctttatacattctggatacaagtcctttatcagatccatgtgtcgt | |
| gaatgttttcttctgatctgttgcttgcctatttgtttgctttacagagtttacagtatcttaagaggagtggatttatcttttttatgttca | |
| gtatttgccttgtcctgtttaggacatctttttttttttttttaaccccagggtcatgaagatattatcttacattttcttttaggaccttta | |
| tggttgtaagttttacagtaaggtccttgagccattaattaattcttaaaattaattgtttatggtgtgaggtgtaggagtcagtctctggt | |
| atctttcctgtatggaaatccagttattctgtctccacttgttgaaataggcttcctttctctactgaatgcttttaattttaattattttac | |
| agttggagtatagggctaccattttagtgctattttctttttttctttgttaatttttgagacagggactcacactgttgcccaggctagagt | |
| acaatggcacaatcaaggcttactgcagcctcgaacccctgggctcaagcagtcctctagcagcctcacgagtagctgggattactccaccac | |
| acccagctaactattttatttttttgtattgacaggatctcactatgttgcccaggctggtctcaaactgctggcctcaagctttcatcccat | |
| ctcggcctcccaaagtgctgggattacaggtgtgagccaccatgcctgacctcttagtgctattttctatttatctcctctgttctctgctct | |
| ctttaaacgttggaggaagaaacagtacccatcttacacaaactcttcagaaaacagaggaacagactgggcgcggtggctcatacctgtaat | |
| ctcagcactttggtacgctgaggcaggggatcatttgaggtcgggagttcgagaccagcctggccaacacggcgaaaccccatctctactaa | |
| aaatacaaaaagtagctaggcgtggtgacacatacctgtaatgccagttactcaggaggctgaggcacaagaatcccttgaacctgggaagcg | |
| gaggttgcagtgagccgagattgcgccactgcactccagcctgggcaacagagtgagaccctgtctcagaaaaaaaaagaaagaaagaaaaaa | |
| tagaggaatatttcccaacttgttttcgaagccagcataatcctggtaccaaaaccaaacaaggacattataagaaaagaaaatatagacca | |
| atattcctgttagcatagacatgcaacagctaaccaattttagcaaaccaaacctggtaatatagaaaaaaggataaataggccagtcgcggt | |
| ggctcacgcctgtaatcccagcactttgggaggctgaggcaggcagatcacttgaggtcaggagtttgagaccagcctgaccaacatggtga | |
| aaccccgtttctaataaaaatacaaaaatcaggctgggcacggtggctcacgcctgtaatcccagcactttgggaggccgaggtgggcaga | |
| tcacgaggtcaggagttcaagaccagcctgaccaatgtggtgaaacgccatctctactaaaaatacaaaaatcagccggtgtggtgg | |
| cacctgcctgtaatcccagctactcaggaggctgaggcagaattgcttgaacccgggaggcagaggttgcagtgagccaagatcgt | |
| gccactgcactccagcctgggcgacagagcaagacttcatctcaaaaaaaaaaaaaaattagctgggcatggtggtgggcacctga | |
| aatcccagctactcgggagtctgaggcaggagaatcgcttgaacccaggaggcagaagttgcactgagctgggatcacaccattgc | |
| actccagcctgggcaacagagtgagactccatctcaaaaaaagaaaaagaaaaaggataaatacattctaaccaaataatgtttatctc | |
| atgattgtagctgattcaacattcaaaaattggcctggtgcagtagctcaggcctgtaatcccaacattttaggaggctgaggcaggaa | |
| gatctcttgagcccaggatttcaagaccagcctgggcaacatagtcagactggtctttactggggggaaaaaaatcagtctgtgtaattc | |
| accacattaacaaagggaaacataaaaaccctatgatcatttcaacagatgtagcaaaagcagttaatgatattcaacacatatgcatga | |
| ttacaaaccaaccaacctcctagcaaactagggaaaggaaacttaacctagtttgataacagggcgtccacagtcggagttccactag | |
| cagcatacataatggtagaaaactcagtgctgccgggcgcggtggctcacgcctgtaatgccagcactttgggaggcctaggcggg | |
| cggatcacgaggtcaggagatcgagactgtcctgactagcatgctgaaaccccgtctctactaaaaatacaaaaacaaaaaattagcc | |
| gggcatggtggcgggcgcctatagtcccagctactcgggaggctgaggcgagagaatggcgtgaacccgggaggcggagcttgc | |
| agagcctagatcgtgccactgcactccagcctgggtgacagagtgagacttcgtctcaaaaaaaaaaaaaaaaaaaaaagaaaaga | |
| aaactcaacgctttttcctctaagatcaggaactagaaaaggatttgactctcacaacgttgataccatactggaggttttaaccaggcaa | |
| gaaaaagaaataatgagggccgggtgcggtggctcaggcctgtaatcccagcactttgggaagccgagacgggtggatcacgagg | |
| tcaggagatcgagaccatcctggctaacacggtgaaaccctgtctctactaaatatacaaaaaattagccgggcgtagtggcgggcg | |
| cctgtagtcccagctactcgggaggctgaggcaggagaatggcgtgaactcagggggcggagcttgcagtgagctgagatcgagc | |
| cactgcactccagcctgggcgacagagcaagactgtgtctcaaaaaaaaaaaaagaaaaagaaataatgattagtggcccgatgtct | |
| cacgcctataatcccagcactttgggaggccgaggtgggcagatcacctgaggtctggagttggagaccagcctgacaaagatggt | |
| gaaacctcgtctctattaaaatattaaaaaaatagccaggcgttggccgggtacagtggctcatgcctgtaatcccagcactttgggag | |
| gccgaggtgggtggatcacctgaggtcaggagttcaacaccagcctggccaacatggtgaaaccccatctctactaaaaatacaaaa | |
| attagccgggcgtagtggcgggcgcctgtaatcccagctacttgggaggcttaggcaggagaatcgcttgaacctgggaggcggag | |
| gttgtagtgagccgagattgcaccattgcactccagcctgggtgacaaaagcaaaaactccgtctcaaaaaaaaaagaattagccag | |
| gggtagtggtgaacgcctgtagtcccagctactcaggaggcagaggcaggagaatcacttgaacccaggaggcagaggttgcagt | |
| gagccgagattgtcccattgcactccagcctaggcgacaagagcaaaattccatgtcaaaaaaaaaaaaaaaaaaggaaagaaaaa | |
| aaataacgattagaaaggaagaaataaaacacattcacagccagtatgattctatacatacatgtcctaatggggccaggcgtggtgg | |
| ctcatgcctgtaatcctagcacttttaggaggctgaggcaggtggcttccctgggaccagcctggccaacatggtgaaaccccaactc | |
| taataaaaatacaaaaaatcagccaggcgtggtgacgggcacctctaatcccagctactcaggaggctgaggcaggagaattgcttg | |
| gacctgggaggcagaggttgcagtgagccgagatcgcgctattgcactccagcctgggcaacaagagtgaaactccggcagggtg | |
| tggtggcttacgcctgtaatcccagcacttcgggaggctgaggcaggccgatcacctgaggtcaggagtttgagaccaacctaacat | |
| ggtgaaaccccgtctctactaaaaatacaagaattagctgggtgtagtggtgggcgcctgtaatcccagctacttgggaggctgagac | |
| agaagaattgcttgaacccaggaggtggaggttgcagtgagctgagatcatgccattgcacaccacgccgggcaacagagcgagat | |
| tccgtctcaaaaaaaaaaaaaaagagtgaaactctatctcaaaaaaaaaaaaaagtcctaatggaaaatccataaaaagctaccaaaa | |
| ctaataaataaatatagcagggttgcaggttacagggcaatatagttatccctctatctgtaggggcttggttctgggactcctcacacac | |
| caaacccacagatgtctaagtcccatatataagacggtatagtatttggatttaacctacacatatcctcccatatagtttaaattatctctag | |
| attacttacattacccccatacaatgaaaatgctaatgtacatgcaagtatgtatgtaagtacttgtactatattgtttagggaatcactggac | |
| atataggccttcaagactgataccagcagccactgttaagattctggtcaggcctgcccctgtttggggtctcagttgatctcattgccttc | |
| ccacccagccaagggcacctgcatttctcttggctccctggccatttggaaggcctagttcagcctggcacatttgtatcctggcccact | |
| gatgctggtacccctgggaaggtcctgctctgaaaaacacggagattttagttgctactgaagatttgagagataaagacagggagac | |
| ctgtctgtagacctgtgtccctccaagtgggattgagactttgggccccccatttcaggacagcacctcctggcctgttgactgaataga | |
| tccctgaaggaggtgtacttgcattaatggagtgggggtgggagcagtaccacagatccgcactaacaatcacacagttctctctaga | |
| ataataatatagaacaagtgaaatagaacaattgcagaaagagctaacctttgttgagctcttactgtgtgcccagcactttcctcaactct | |
| acatttcccataatacacagagtactaggtaggccaggcttggtggctcacgcctgtaatcccagcactttaggaggccaaggggggt | |
| ggatcacctgaggtcgggagttcaagaccagcctgaccaacatggtgaaaccccgtctctactagaagtacaaaattagccaggtgt | |
| ggtggcacatgcttgtagtcctagctactcagcaggctgaggcaggagaatcatttgaatccgggaggaggttgcagtaagcggaga | |
| tagtgccactgtactccagcctgggcaataagagctgagactccgtctcaaaataaaataaaataaaataaaataaaataaaataaaat | |
| aaaaaaagaaaagagcctgccattaaaggagctgtttggtaggggatgttttgtcagtgcaaacaacagaaaagtgggctgggcaca | |
| gtggttcatgcctgtaatcccagcactttgggaggccaaggcgggcggatcacctgaagttgggagttcaagaccagcctgaccaat | |
| atggagaaaccccgtctctactaaaaatacaaaattagccgggcgcagtggcgcatgcctgtaatcccagctactcgggaggctgag | |
| gcaggagaatcgcttgaacctgggaggcagaggttgcggtgagccgagatcgcaccattgcactccagcctggacgagagcaaaa | |
| ctctgtctcaaaaaaaaaaaaaaacagaaaagtgtaacaaacacttacagtaggcatgtttcttagcaaatctgatgacaaatttggcat | |
| aaagaaagagagcatccctgaaaaaaaaaaaaagaaaaagaaagagagcatcctgcctgggcaacatagtgaaaccctgcctcta | |
| caaaaaaactcaaaaattggccgggtgcagtggctcacacctgtaatcccagcactttgggagtcggaggcgggaggatcacctga | |
| ggtcaggagttcgaaaccagcctggccaacatggcaaaaccccatctctactaaaaatacaaaaaattaatcaggcgcattggtggg | |
| cgcctgtaatcccagctactcaggaagttgaggcaagaggatcgcttgaatctgggaggtggaggttacagtgagtcgagatcacac | |
| cactgcactctagcctgggtgacagggcgagactccgtctccaaaaaaaaaaagaaaaagaaaaagactaaaaaattagccaggca | |
| ggcctctgtggtcccagctacttgggaggctgaggcaggagaatcactgagcccaggagtccgaggctgtagtgagccatgattgc | |
| accactgtaccctagcttgggcaacaaagcaagaccctgcctcaaaagaaaaaagaaagaaagaaagaacatggcgggccaggc | |
| acagtggctcacacctgtaatcccagcgctttgagaggccgaggcaggtggatcacaaggtcaggagttccacaccagcctggcca | |
| acatggtgaaaccctgtctctactaaaaatacaaaaaatcagccaggcatggtggcaggggcctgtaatcccagctactcgggaggc | |
| tgaggcaggagaattgcttgaaaccagaaggcagaggttgcagtgagcctagactgcaccactgcactccagcctgggcgaaaag | |
| agccaaactccatctcaaaaaacaaacaaaaaaacaaaacaaaagaaaacatggcaaagcctttgaaagcttgtctgggagaaggt | |
| gcgatgatagttgcataacttcgtgcaagatgctggtccacacaggggctgccccttgctctttctcgctctcttaacctctcatataacag | |
| gcttgtgtgttattcacatttattgagcccaagcaggtgcaaggcattgtgatctaatactttggtcagcaagacaacaagatagatcact | |
| gccctgcccttaggaagtgtatatgctattagaggaaacagataaaataaacaaggaaaagtatcagacaatgtaagtgctatgagaat | |
| gcaaatgaggtgatgtgaattaaaataggatgacttaaagtctgcacgggaaggagcctacccccatgttcctggctagccaaggaac | |
| caccagttgattagcagagaagggcagccagtctagctagagcttttggggaagagggagtggttgttaagagatgagattaaagaa | |
| gccgagacgggccattcgtgaggggtttgtaatgcagggctgaggagtgtccgaagagaatgggcaggtgagcggtgagacagtt | |
| gttcttccagaagctttgcagtgaaaggaatcaaagaaatggagccgtgtatcaggtggggaagggtgggggccaagggggtgtcc | |
| ttccccatacagagattgcaggctgagaatgactatatccttgttaacaggaggtgggagcagggcacggtagctcacacctgtaatct | |
| tggcactttaggaggctgaggcgggccgatcacctgaagtaaggagttcgagaccagcctggccaacatgcaaagccctgtctctac | |
| taaaaatacaaaaattagctgggtgtggtggtactcgcctgtaatcccagctactcgggagactgaggcaggagaatggcttgaaccc | |
| ggaaggtagaggttgcagtgagctgagatcatgccactgtgctccagcctaggtgacagagagagactccatctcaaaaaaaaaaa | |
| aaaaatacaggaagggagttgggaatagggtgcacatttaggaagtcttggggatttagtggtgggaaggttggaagtccctctctga | |
| ttgtcttttcctcaaagaagtgcatggctggtgaggggtggggcaggagtgcttgggttgtggtgaaacattggaagagagaatgtga | |
| agcagccattcttttcctgctccacaggaagccgagctgtctcagacactggcatggtgttgggggagggggttccttctctgcaggcc | |
| caggtgacccagggttggaagtgtctcatgctggatccccacttttcctcttgcagcagccagactgccttccgggtcactgccatgga | |
| ggagccgcagtcagatcctagcgtcgagccccctctgagtcaggaaacattttcagacctatggaaactgtgagtggatccattggaa | |
| gggcaggcccaccacccccaccccaaccccagccccctagcagagacctgtgggaagcgaaaattccatgggactgactttctgct | |
| cttgtctttcagacttcctgaaaacaacgttctggtaaggacaagggttgggctggggacctggagggctggggacctggagggctg | |
| gggggctggggggctgaggacctggtcctctgactgctcttttcacccatctacagtcccccttgccgtcccaagcaatggatgatttg | |
| atgctgtccccggacgatattgaacaatggttcactgaagacccaggtccagatgaagctcccagaatgccagaggctgctcccccc | |
| gtggcccctgcaccagcagctcctacaccggcggcccctgcaccagccccctcctggcccctgtcatcttctgtcccttcccagaaaa | |
| cctaccagggcagctacggtttccgtctgggcttcttgcattctgggacagccaagtctgtgacttgcacggtcagttgccctgagggg | |
| ctggcttccatgagacttcaatgcctggccgtatccccctgcatttcttttgtttggaactttgggattcctcttcaccctttggcttcctgt | |
| cagtgtttttttatagtttacccacttaatgtgtgatctctgactcctgtcccaaagttgaatattccccccttgaatttgggcttttatccat | |
| cccatcacaccctcagcatctctcctggggatgcagaacttttctttttcttcatccacgtgtattccttggcttttgaaaataagctcctga | |
| ccaggcttggtggctcacacctgcaatcccagcactctcaaagaggccaaggcaggcagatcacctgagcccaggagttcaagaccagcct | |
| gggtaacatgatgaaacctcgtctctacaaaaaaatacaaaaaattagccaggcatggtggtgcacacctatagtcccagccacttag | |
| gaggctgaggtgggaagatcacttgaggccaggagatggaggctgcagtgagctgtgatcacaccactgtgctccagcctgagtga | |
| cagagcaagaccctatctcaaaaaaaaaaaaaaaaaagaaaagctcctgaggtgtagacgccaactctctctagctcgctagtgggtt | |
| gcaggaggtgcttacgcatgtttgtttctttgctgccgtcttccagttgctttatctgttcacttgtgccctgactttcaactctgtctcctt | |
| cctcttcctacagtactcccctgccctcaacaagatgttttgccaactggccaagacctgccctgtgcagctgtgggttgattccacaccccc | |
| gcccggcacccgcgtccgcgccatggccatctacaagcagtcacagcacatgacggaggttgtgaggcgctgcccccaccatgag | |
| cgctgctcagatagcgatggtgagcagctggggctggagagacgacagggctggttgcccagggtccccaggcctctgattcctca | |
| ctgattgctcttaggtctggcccctcctcagcatcttatccgagtggaaggaaatttgcgtgtggagtatttggatgacagaaacacttttc | |
| gacatagtgtggtggtgccctatgagccgcctgaggtctggtttgcaactggggtctctgggaggaggggttaagggtggttgtcagt | |
| ggccctccaggtgagcagtaggggggctttctcctgctgcttatttgacctccctataaccccatgagatgtgcaaagtaaatgggttta | |
| actattgcacagttgaaaaaactgaagcttacagaggctaagggcctcccctgcttggctgggcgcagtggctcatgcctgtaatccc | |
| agcactttgggaggccaaggcaggcggatcacgaggttgggagatcgagaccatcctggctaacggtgaaaccccgtctctactga | |
| aaaatacaaaaaaaaattagccgggcgtggtgctgggcacctgtagtcccagctactcgggaggctgaggaaggagaatggcgtg | |
| aacctgggcggtggagcttgcagtgagctgagatcacgccactgcactccagcctgggcgacagagcgagattccatctcaaaaaa | |
| aaaaaaaaaaggcctcccctgcttgccacaggtctccccaaggcgcactggcctcatcttgggcctgtgttatctcctaggttggctct | |
| gactgtaccaccatccactacaactacatgtgtaacagttcctgcatgggcggcatgaaccggaggcccatcctcaccatcatcacact | |
| ggaagactccaggtcaggagccacttgccaccctgcacactggcctgctgtgccccagcctctgcttgcctctgacccctgggccca | |
| cctcttaccgatttcttccatactactacccatccacctctcatcacatccccggcggggaatctccttactgctcccactcagttttctttt | |
| ctctggctttgggacctcttaacctgtggcttctcctccacctacctggagctggagcttaggctccagaaaggacaagggtggttgggag | |
| tagatggagcctggttttttaaatgggacaggtaggacctgatttccttactgcctcttgcttctcttttcctatcctgagtagtggtaatc | |
| tactgggacggaacagctttgaggtgcgtgtttgtgcctgtcctgggagagaccggcgcacagaggaagagaatctccgcaagaaagg | |
| ggagcctcaccacgagctgcccccagggagcactaagcgaggtaagcaagcaggacaagaagcggtggaggagaccaagggt | |
| gcagttatgcctcagattcacttttatcacctttccttgcctctttcctagcactgcccaacaacaccagctcctctccccagccaaagaag | |
| aaaccactggatggagaatatttcacccttcaggtactaagtcttgggacctcttatcaagtggaaagtttccagtctaacactcaaaatg | |
| ccgttttcttcttgactgttttacctgcaattggggcatttgccatcagggggcagtgatgcctcaaagacaatggctcctggttgtagcta | |
| actaacttcagaacaccaacttataccataatatatattttaaaggaccagaccagctttcaaaaagaaaattgttaaagagagcatgaaa | |
| atggttctatgactttgcctgatacagatgctacttgacttacgatggtgttacttcctgataaactcgtcgtaagttgaaaatattgtaagtt | |
| gaaaatggatttaatacacctaatctaaggaacatcatagcttagcctagcctgctttttttttttttttttttggagacagagtctcactct | |
| gtcacccaggctggagtgcagtggcgggatctcggctcactgcaacctccgccttctgggttcaagcgattctcctgcctcagcccactga | |
| gtagctgggattacaggcacctgccccgacgcccagctaattttttgttatttatttatttttttttttagtagagatgaggtttcaccatgt | |
| tggccaggctagtctcgaactcctgaccttgtgatctgcctgccttggcctcccaaagtgctgggattacaggcgtgagccaccgcacccg | |
| gcctgcctagcctacttttattttatttttaatggagacagcatcttgctctgttgcccaggctggattacagtgatgtgatcatagctcatta | |
| taccctcctgggctcaagcaatccccctaactctgcctccccagtagctaggaccacaggcatacaccaccatacccagctaatttttaa | |
| aattttttgtagatagatagagtctcactatgttgcccaggctggtctctagcctacttttttgagacaaggtcttgctctgtcacccaggctg | |
| gatagagtgcagtagtgcagtcacagctcactgcagcctccacctcccaggctccatccatcctcccagctcagcctcccaagttgctt | |
| caactacaggcctgcaccaccatgcctggctaatttttatttatttatttttattttattttattttatttttttgagactcagtctcactct | |
| gtcgcccaggctggagtgcagtggcatgatctcggctcactgcaacctctgcctcctgggttcaagtgattctcctgcctcagcctcccgaat | |
| agctaggactacaagcgcctgctaccacgcccagctaatttttgtatttttagtagagacagggtttcaccatgttggccaggctggtctcga | |
| acttctgaccatgtgatccgcccgcctcggcctcccaaagtgctgggattacaggtgtgagccaccacgcccggctaatttttatttattta | |
| tttaaagacagagtctcactctgtcactcaggctagagtgcagtggcaccatctcagctcactgcagccttgacctccctgggctccgg | |
| tgatttcaccctcccaagtagctaggactacaggcacatgccacgacacccagctaattttttattttctgtgaagtcaaggtcttgctacg | |
| ttgcccatgctggtatcaaacccctgggctcaatcaatccttccacctcagcctccccaagtattggggttacaggcatgagctaccaca | |
| ctcagccctagcctacttgaaacgtgttcagagcatttaagttaccctacagttgggcaaagtcatctaacacaaagccctttttatagtaa | |
| taaaatgttgtatatctcatgtgatttattgaatattgttactgaaagtgagaaacagcatggttgcatgaaaggaggcacagtcgagcca | |
| ggcacagcctgggcgcagagcgagactcaaaaaaagaaaaggccaggcgcactggctcacgcctgtaatcccagcatttcggga | |
| ggctgaggcgggtggatcacctgaggtcaggagttcaagaccagcctagccaacatggtgaaaccccgtctctactaaaatacaaaa | |
| attaaccgggcgtgatggcaggtgcctgtaatcccagctacttgggaggctgaggcaggagaatcgcttgaaccaggaggcggag | |
| gttgcagggagccaagatggcgccactgcactccagcctgggcgatagagtgagactccgtctcagaaaaaaaagaaaagaaacg | |
| aggcacagtcgcatgcacatgtagtcccagttacttgagaggctaaggcaggaggatctcttgagcccaagagtttgagtccagcctg | |
| aacaacatagcaagacatcatctctaaaatttaaaaaagggccgggcacagtggctcacacctgtaatcccagcactttgggaggtgg | |
| aggtgggtagatcacctgacgtcaggagttggaaaccagcctggctaacatggtgaagccccatctctactaaaaacacaaaaattag | |
| ccaggtgtggtagcacacgcctgtagtcccagctactcgggaggctgaggcacaagaatcacttgaaccccagaggcggagattgc | |
| aatcagccaagattgcaccattgcactcccgcctgggcaacagagtgagaccccatctcaaaataaataaataaatatttttaaaagtca | |
| gctgtataggtacttgaagtgcagtttctactaaatgcatgttgcttttgtaccgtcataaagtcaaacaattgtaacttgaaccatctttt | |
| aactcaggtactgtgtatatacttacttctccccctcctctgttgctgcagatccgtgggcgtgagcgcttcgagatgttccgagagctgaat | |
| gaggccttggaactcaaggatgcccaggctgggaaggagccaggggggagcagggctcactccaggtgagtgacctcagcccctt | |
| cctggccctactcccctgccttcctaggttggaaagccataggattccattctcatcctgccttcatggtcaaaggcagctgaccccatct | |
| cattgggtcccagccctgcacagacatttttttagtcttcctccggttgaatcctataaccacattcttgcctcagtgtatccacagaacatc | |
| caaacccagggacgagtgtggatacttctttgccattctccgcaactcccagcccagagctggagggtctcaaggaggggcctaata | |
| attgtgtaatactgaatacagccagagtttcaggtcatatactcagccctgccatgcaccggcaggtcctaggtgacccccgtcaaact | |
| cagtttccttatatataaaatggggtaagggggccgggcgcagtggctcacgaatcccacactctgggaggccaaggcgagtggatc | |
| acctgaggtcgggagtttgagcccagcctgaccaacatggagaaaccccatctctactaaaaatacaaaagtagccgggcgtggtga | |
| tgcatgcctgtaatcccagctacctactcgggaggctgaggcaggagaatcgcttgaacccgggaggcagaggttgcggtgagctg | |
| agatctcaccattacactccagcctgggcaacaagagtgaaactccgtctcaaaaaagataaataaagtaaaatggggtaagggaag | |
| attacgagactaatacacactaatactctgaggtgctcagtaaacatatttgcatggggtgtggccaccatcttgatttgaattcccgttgt | |
| cccagccttaggcccttcaaagcattggtcagggaaaaggggcacagaccctctcactcatgtgatgtcatctctcctccctgcttctgt | |
| ctcctacagccacctgaagtccaaaaagggtcagtctacctcccgccataaaaaactcatgttcaagacagaagggcctgactcaga | |
| ctgacattctccacttcttgttccccactgacagcctcccacccccatctctccctcccctgccattttgggttttgggtctttgaacccttgc | |
| ttgcaataggtgtgcgtcagaagcacccaggacttccatttgctttgtcccggggctccactgaacaagttggcctgcactggtgttttgt | |
| tgtggggaggaggatggggagtaggacataccagcttagattttaaggtttttactgtgagggatgtttgggagatgtaagaaatgttctt | |
| gcagttaagggttagtttacaatcagccacattctaggtaggggcccacttcaccgtactaaccagggaagctgtccctcactgttgaat | |
| tttctctaacttcaaggcccatatctgtgaaatgctggcatttgcacctacctcacagagtgcattgtgagggttaatgaaataatgtacat | |
| ctggccttgaaaccaccttttattacatggggtctagaacttgacccccttgagggtgcttgttccctctccctgttggtcggtgggttggt | |
| agtttctacagttgggcagctggttaggtagagggagttgtcaagtctctgctggcccagccaaaccctgtctgacaacctcttggtgaa | |
| ccttagtacctaaaaggaaatctcaccccatcccacaccctggaggatttcatctcttgtatatgatgatctggatccaccaagacttgtttt | |
| atgctcagggtcaatttcttttttcttttttttttttttttttctttttctttgagactgggtctcgctttgttgcccaggctggagtggagt | |
| ggcgtgatcttggcttactgcagcctttgcctccccggctcgagcagtcctgcctcagcctccggagtagctgggaccacaggttcatgcca | |
| ccatggccagccaacttttgcatgttttgtagagatggggtctcacagtgttgcccaggctggtctcaaactcctgggctcaggcgatccacct | |
| gtctcagcctcccagagtgctgggattacaattgtgagccaccacgtccagctggaagggtcaacatcttttacattctgcaagcacatc | |
| tgcattttcaccccacccttcccctccttctccctttttatatcccatttttatatcgatctcttattttacaataaaactttgctgccacct | |
| gtgtgtctgaggggtgaacgccagtgcaggctactggggtcagcaggtgcaggggtgagtgaggaggtgctgggaagcagccacctgagt | |
| ctgcaatgagtgtgggctggggggcccagtgcccgggttccgggaggggaacaaaggctggagactgggtcagtctgcgggctg | |
| catgacaacaagggagggggtggctccattcataactcaggaaccaaccgtccctcctcccctccggccacggctggcacaaggtt | |
| ctctccctcccctgcttctaggactgggctgcttccccctcggcagcctctcaccaaggattacgggatttaaatgtctgatttagcaagg | |
| ctgagcctccagggtggccatctgctccatcagaaagtggcaggatacctgggttcccaaggggaacaggggtgggtgctactgga | |
| tggagagaggccagtgggaggcctgctagccagggtcccaggaaagtgggggcagctaaggtaagagtaggggtgtggggcta | |
| ggtccttcccagcatcccctcatcctgggcctcatgccaggtagctgaatgaattgaagctttaaactctgccaggaaaacctttcaaag | |
| ggcttcttgggatagggaggagagtcgggttgaggagctcagtactgcctgcccatgctcctcagggctgctggctcccagggagg | |
| ggggctgggagcaggcaggctcttccccatcacccactgctctcttggagccagtgcttgaaggggcagtcagacatggcttgccct | |
| tcctcctccctggtggtggagatgggtgttagggtccagtgggtgctactgtccaggggggcttctggggccaccagcctgtcagctc | |
| atcaaccaggctgaaggtgcaagcaggagccccttgccttgccccaaggatcccagacagctatgaagccaccagccttcctgacct | |
| caagaccaccttttttttttctctttcttactagggaatgccaaacactctccccaggagatccagacccgcctctttcagagacttttaactt | |
| aaacatctgtccctacccagcaggcaaactagagctcctgaagctcagtccctgtccttgcctctgtagacaggtcaccttgatgagctt | |
| cctttttttttttttaatttttttttattttaggctttattggggcataattgatcccccaaaattgcatacattcaaggtatgcagtgtgatg | |
| atttgatatgggggtatattgtgaaaccattaccacaatcaaattaatcagcacgtccatcatcacacacagttaccatttgtgtgtgtgcac | |
| gtgtgttcacctacgacgaggacacttggacctactctgcagatctcaagtaaacagaaaatctccctttttgacaaccatcctccacccttt | |
| caatcccaaccttttcctagattatgtccctagctctgtttttatttctgctgtgctgcttcagatccattctgactctgccaaacccttctt | |
| tgtgagctgatagattgctggattgagaattacagctgggcgcggtggctcacgcctgtaatcccaacactgtgggaggccaaggccggcggat | |
| cacttgaggtcaggagttggagaccagcctgaccaacaagatgaaaccccatctctactaaaaatacaaaattagctgggcatggtgg | |
| tgcacgcctgtaatctcatcttcttgggaggctgaggcaggagaattgcttgaacccgggaggtggaggttgcagtgagccaagatcc | |
| tgccattgcactccagcctgggcaacaacagtgaagctccatctcaaaacacacaaaaaaaagaagtacaaagtctgagacttcagg | |
| ccagctctgctacactatatactctaacctctctggtcctacttggtgacttctttccctctggtcgtgttcaagttcccgtcccatccagtca | |
| agcaggtactcattggtaccttaccctgtgccaggagctgttctaggccctggaaacctatggcagacatgttccctaccctcccactca | |
| aagagcccaggccttatcctaatgagatctgaaatcaaatctcccaatttcctcatggcttcagtctaaacttgtaattcacaaccttaaatc | |
| aatatgttctatttttttatttagaaaacatttccggccaggcacggtggatcacacctgtaatcccagctactcgggaggctgaggcagg | |
| agaatcgcttgaacccaggaggcagagggttgcagtgagccgagattgcgccattgcactctagcctgggcaacagagcaagactc | |
| catctcaaaaaagaaaaaaaaatggaagaaaaaaaaatttccccctcattttaggaacacgaggtctccaaatctaaaattcgtactctg | |
| aggagattgaatagccttaaatgctttcatcattaaaaagaaaagaaaggaacctggtatgcatcctaaaaatgaaaaatatacctacct | |
| gtaatcccagcacacagcacattgggaggctaaagcaggaggataacttgaggccaggagtttcagatcagcctgggcaacatagc | |
| aacaccccatttctttttcttttcttttttttttggagacacagtctcgctctgttactcaggctggagtgcagtggctcaatctcagctcac | |
| tgcaagctctgcctcccaggttcatgccattctcctgcctcagcctcccgagtagctgggactacaggcgcccgccaccacgcctggctaa | |
| ttttttgtatttttagtagagacagggtttcaccgtgttagccaggatggtctcgatctcctgacctcgtgatccgccagccttggcctccta | |
| aagtactgggattacaggcgtgagccactgcgcctggccacaacaccccatttctattttaataaaataaaatactgtgaaaaacatttac | |
| aatttttaaattttaattttaaaattaaacttatatttattcatttgtgtgtgtgggtttttttttttttttttttttgctttttttttgaga | |
| tggagtgtcactctgtcacccaggctggagtgcagtggcgtgatctctgcctcccggttcaagtgattctcctgccatagcctcccaagtagc | |
| tgggactacaggtacacgccaccacgccgggttaatttttgtatttttagtagagacaggatttcactgtgtcgccaggctagcctcgaact | |
| cctgacctcaggtgattcgcccaccttggcctcccaaagtgctgtgattacaagcgtgagccaccgtgcccagcccaaagttggttttaatag | |
| cagaaaatctatcaacataattcaatatattaaatttagaaagaaaaattatctatcatatcaacagatactgaaaggaatttgattaaattt | |
| cagtagccatttccttaaaaaagaaaacactttaacacagtaatagactgataatggaataccaattttcctaataagttaaacattaagata | |
| atttcaattaaggtcaagagctgggccaggtgcagtggctcacacctgtaatcccaacactttggaggccaaggtgggtggatcacctgagg | |
| tcaggagtggagaccagcctggctgacaatagtgaaatcctgcctctactaaaaacacaaaaaattagctgggcatggtggtgggca | |
| cctataatcccagctactgggaaggctgagacaggagaattgcttgaacctgggaggcggaggttgcagtgagcaaagatcacacc | |
| attgcactccagcctgggcgacagagccagagtcagtctcaaaaaaaaaaagaggtggccacacctataatccaaacattttgtgag | |
| gccaaggcaggagaattgcttcaggccaagagttgaacacctcgtcaacatagccagacctctctctagatagatagatagatgatag | |
| atagagagatagatagatgatagatagagagatagatagatgatagatagatagatagatagatagatagatagatagatagatagata | |
| gatagatagataatctggccgggtgtggaggctcacgcctgtaatcccagcactttgggaggctgaggcgggcagatcacgaggac | |
| aagagattgaaaccatcctggctaacaaggtgaaaccccgtctctactaaaaatacaaaaaattaggcgggtgtggtggcacgcgcct | |
| gtagtcctagctattcaggaggctgagacaggagaattgcttgaatccgaaaggcggaggttgcagcgagccgagatcgtgccact | |
| gcactccagcctgggtgacagagcaagactccatctcaaaataaataaataaataatcaagaacagtataaggggctgtatggtggct | |
| catgcctgtgatcccagcactttgggaggccaaggtgggaggatcccttgagaccagcccaggcaacagagaaagaccctgtctct | |
| atttaaaaaaattaaaaactggccgggcacggtggctcacgcctgtaattccagcgcttgggaggccaaggcaggcacatcaggag | |
| gtcaggagttcgagaccagcctggccaacgtggtgaaaccccgtctctactaaaaatacaaaaagtagctaggcgtggtggcaggc | |
| acctgtaatcccagctacttgggaggctgaggcaggagaatcgcttgaacccaggaggcggaggttgcagtgggcaaagatcgtgc | |
| cattgcactcagcctgggtgacagggcaagactccatctcaaaataaataaacaaagtaattaattaattaaattaaaaactgtggggat | |
| atagacttactctggttttattttttcttttcttttcttttcttttttctgagacggagtctcgctctgttgcccaggctggagtacagtgg | |
| cgtggtttctgttctctgcaacctccacctcccggattcaagcgattctcttgcctcagcctcttgaatacctggaattacaggtgcctgcc | |
| accacccccggctaattttttgtatttttagtagagacagggtttcaccatgttggccaagctggtctcgaactcctgacctcatgatccac | |
| ccgcctctgcctcccaaagcactgagactacaggagtgagccactgtgcccagcctactctggttttagtgcattcaagaggaacaaaaaagga | |
| agaaaatcactagtaaatatacctctttctggttagagtggatgtttggaaattatatatatattatattatattatatatattatatatata | |
| cacaaacacgtacatacatgcacacacatatatgcctttttgattataggatagtataccaaaactcagaaatattatggaattaacagaatt | |
| tagtaaggcagataagtagtaggtagaaaaatattaattttatcttccagcagaagcactgtgaaaaattagacaacaagaaaacattccattc | |
| aaaataatgacaataaggccgggcatggtggctcacacctgtaatcccagcactttgggaggctgaggcaggaggatcatctgaggt | |
| caagtttgagatcagcctggccaacatggtgacaccctgtctctactgaaaatacaaaaatcagccagctatggtagtgtaagcctgta | |
| attccagctactcgggaggtcgaagcagaagaatcacttgaacccaggaggcagagattgcagtgagccaagatcctgccagtgctt | |
| tccagcctgggcaacagtgtgaggctccatctcaaaaaaaaaaaaaaaaaaaagacaatagcaataaacattaagaaatgtgtaatag | |
| gaatggcacacacaaagaaggaatggcacagagcctgtatgcagaagaccacaaacccttatttaacgacgtaagccaagatccaa | |
| agaaaatgatagattctcagatgggaaaactaaaaaaataagaaaaatcaattatctcgagataaatataatataatgcaatttcaattag | |
| aatcccaaattttcattgtgtgtgtgtgtgagttgggtaaatttatcataaatgtataggaacgagtaagtgtcactagttgtttaaataaata | |
| ctggatttgggccaggcatggtggctcacgcctctaatcccagcactttgggagaccgaggcgggcagatcatgaggtcaggagat | |
| cgagaccatctggccaacatagtgaaaactcgtctctactaaagatacaaaaaattagctgggcatggtggcacgtgcctgtagttcca | |
| gctactctggaggctgaggcaggagagttgcttgaacccgggaggtggaggttgcaatgagccgagatcctgtcactgcactccac | |
| cctggcgacaaagtgagactccgtctctctctctctctttaggccaaggcaggtggatcacctgaggtcaggagttcaagacagcctg | |
| gccaacatagcgaaatcccatctctactaaaaatacaaaaattagcctggcagtggtggcccacgcctgtaatcccagctactaaggg | |
| ggctgaggcaggaggatctcttaaccagggaggaggaggttgcagtgagcagagattgtgccactgcactccagcctgtgcaacag | |
| agtgagactctgtctcaaaaaaaataaataaacaaaatactggaggccgggcacggtggctcccgcctgtaatcccagcactttggga | |
| ggccaaggcgggtgaatcgctttcagctcaggagttccggaccagtctgggcaacatggcaaaaccccgtctatactaaaaatacaa | |
| aacttagccaggcgtggtagtgcatacctataatcccagctactcgagaggctgaggcaggagaatcccttgaaaccgggaggcag | |
| aggttgcagtaagctgaaatcgtgccactgcactccagcctggacgacacagcgggagactgtctcaataaataaaataaataatata | |
| aaataacataaataataaaattgtaaataataagtaaataataagcaacagaatggagagggggtcctatttgccttgccagattttagag | |
| aacttagtataggctaggcaggtgcagtggctcacgcctgtaatcccagcactttgggagtccaaggcagtggatcacatgaggtcag | |
| gagttcaagaccagcctgaccaacatggtgaaaccccatctctactaaaaatacaaaattagccaggcatggtggcacatgcctgtaa | |
| Protein: | SEQ ID |
| MEEPQSDPSVEPPLSQETFSDLWKLLPENNVLSPLPSQAMDDLMLSPDDIEQWFTED | NO: 347 |
| PGPDEAPRMPEAAPPVAPAPAAPTPAAPAPAPSWPLSSSVPSQKTYQGSYGFRLGFL | |
| HSGTAKSVTCTYSPALNKMFCQLAKTCPVQLWVDSTPPPGTRVRAMAIYKQSQHM | |
| TEVVRRCPHHERCSDSDGLAPPQHLIRVEGNLRVEYLDDRNTFRHSVVVPYEPPEVG | |
| SDCTTIHYNYMCNSSCMGGMNRRPILTIITLEDSSGNLLGRNSFEVRVCACPGRDRRT | |
| EEENLRKKGEPHHELPPGSTKRALPNNTSSSPQPKKKPLDGEYFTLQIRGRERFEMFR | |
| ELNEALELKDAQAGKEPGGSRAHSSHLKSKKGQSTSRHKKLMFKTEGPDSD | |
| CDKN2A | SEQ ID |
| gcccgcgaggtttaggacggatccaggcagaccgcaggctccgggtcggggcaccgggtcagcgcgccggcctgaaggcggc | NO: 308 |
| gtcctgggctcgacttcccgcgcgcggagagccggcgagcccgcgtccgagttcctggacgagagccgagcctcgcttagaccgc | |
| gctcaggacccggctcctccgcattctccggctgcccctgtgtcctcgactcacccctcctttctgccgctccttcctttccttgccctgct | |
| tttactgttcccaaacaggaccgcttttcctgtctcccagctggaaaggaggaagggagagagtccagaaaggatcggtgatgtggaa | |
| gaaaaggggaggaggggacatggagggggagaccggagagagaacgtacgccgaggagtcaggcggcgggatcaagggga | |
| gtcggggtgtctgggcgcggggcagagcgtggaggcggcagcggccaacggtcgccaagacaaccattctacgcgaggacgcg | |
| gcgacaggaggggagcggccagcaggggaggggagcgcgggggaagaggaaagaggaagaagcgctcagatgctccgcgg | |
| ctgtcgtgaaggttaaaaccgaaaataaaaatgggctagacacaaaggactcggtgcttgtcccagccaggcgccctcggcgacgc | |
| gggcagctgggaggggaatgggcgcccggacccagctgggacccccgggtgcgactccacctacctagtccggcgccaggccg | |
| ggtcgacagctccggcagcgccagcgccgcgccgtgtccagatgtcgcgtcagaggcgtgcagcggtttagtttaatttcgcttgtttt | |
| ccaaatctagaagaggagcggagcggcttttagttcaaaactgacattcagcctcctgattggcggatagagcaatgagatgacctcg | |
| ctttcctttcttcctttttcatttttaaataatctagtttgaagaatggaagactttcgacgaggggagccaggaataaaataaggggaatag | |
| gggagcggggacgcgagcagcaccagaatccgcgggagcgcggctgttcctggtagggccgtgtcaggtgacggatgtagctag | |
| ggggcgagctgcctggagttgcgttccaggcgtccggcccctgggccgtcaccgcggggcgcccgcgctgagggtgggaagatg | |
| gtggtgggggtgggggcgcacacagggcgggaaagtggcggtaggcgggagggagaggaacgcgggccctgagccgcccgc | |
| gcgcgcgcctccctacgggcgcctccggcagcccttcccgcgtgcgcagggctcagagccgttccgagatcttggaggtccgggt | |
| gggagtgggggtggggtgggggtgggggtgaaggtggggggcgggcgcgctcagggaaggcgggtgcgcgcctgcggggc | |
| ggagatgggcagggggcggtgcgtgggtcccagtctgcagttaagggggcaggagtggcgctgctcacctctggtgccaaaggg | |
| cggcgcagcggctgccgagctcggccctggaggcggcgagaacatggtgcgcaggttcttggtgaccctccggattcggcgcgc | |
| gtgcggcccgccgcgagtgagggttttcgtggttcacatcccgcggctcacgggggagtgggcagcgccaggggcgcccgccgc | |
| tgtggccctcgtgctgatgctactgaggagccagcgtctagggcagcagccgcttcctagaagaccaggtaggaaaggccctcgaa | |
| aagtccggggcgcattcggcacttgttttgtttggtgtgatttcgtaaacagataattcgtctctagcccaggctaggaggaggaggaga | |
| taaccgccggtggaggcttccccattcgggttacaacgacttagacatgtggttctcgcagtaccattgaacctggacctcccttcacac | |
| agcccctcaatcgtgggaaactgaggcgaacagagcttctaaacccacctcagaagtcagtgagtcccgaatatcctgggtgggaat | |
| gactaagacacacacacacacacacacacacacacacacacacacacacacacagtaggaaaggtgtatttcaagcacactttctttc | |
| tccttggggagaattattgctaaccatctaagttttctggaggcggccttttttctccccagcctcccggcggggtcaccctctcccacctt | |
| ccaggagagtggaggacccgtgagatacggggcacgcaggcagcgacttcctgaaatgctaacaaggatcgtaggatcagttact | |
| gctgcgaggagcaagcacttgcttcttgggggagttttgcagccaacagggaaatgggctttctttgtgagttagaggtagaggtccg | |
| gcggcctgagtgattgaaactgctcgggacaatgctcgtatgtttagcaaacgacagaactgtagaactgttcctgagaaatcccaact | |
| gatagtattttagtcatctcagacgacagttagcacagtttaaaaatgaggcctacttcttgaaaaacagaatccaaggtagttttgtcctc | |
| acattgacaaatgttgacacagccagtgtaatttcctataaccaggaaaactgaaagaatatatgtacagttaaaatatgtacaatgctaa | |
| ttaaaacttgtgtaataagtctaaaagtaatttaatgaggcttcacttttatgaccgtccttgtggtatgcttcgccaggaatatatagcttca | |
| aaaagcaaaggccagcggaggggtaattatttttttactgcaatgttaattgtctctttgacatggaaatataaacctgttaaaactatcagt | |
| gtttaatttagtgtctcaatttctattagcaaaaatttataatctataggataaatgcacattttattttttacttttcatattatgcaagt | |
| taatttttttaatttagtcaaaggagcttataaaggatttcagggcctgttgctggatttgattttaattcattttgaaacattgacaagac | |
| cctggttgttgttttttttaacagtggtttatccgtatcagcaaaagtttagccactgtgaccggtaactgtatgaatatagttcttaatat | |
| tattgtctatataaaaatatttattactctagttaatattattctatataaaatcattttgtttaaattattaagttgcctctgaaaatctg | |
| tagtaacaaagtagaacatgtcaatgtatataaatgccataattatgtattttttagtttaggcctataaaacataacattgtggtgatttt | |
| aagttagagaaaatattttatagtatgttaatgtatatgcatgaaatgcaaaaatatttaaatgataggttcattgaaatagatcatttttt | |
| gttatttaggtataaatcaattttcaggacgtatgtgaaaagcgcaatcttcaggaagtttctcaagatagaacacagcttggatagaatgt | |
| cttgaaatatatgcaattttccaatttcatatgtaaaatgatatacataatataaaatctagcggtgttaattataatgatatgtaattata | |
| tatttcacattaatatattttatgcccatggctatattgatttgggaatatatatggatactaattatgttaggattcatacaattccttga | |
| gaggcacaagtgctaaaaattacttgtatgaattatttaatatcattgcaaataagatgttattttaactttttttaagtttctgcaaatat | |
| gtttattatgactttttatttttatatgattggaaatacatatactaaaattccacgttaccagtttcttaaccacagaaacctgaaaaatt | |
| gccatagttgatttgttacttctaccttggtgcatttacaaaatagtcatatttttattatgaagttaaatattcatttgtttatagctact | |
| tcagaaggctcaggttatttttttctttaatagcacagagtcctctcaaggtaagcactgtgcagttagtataaaccattattccccatgtg | |
| tacatgattcacagtttgtattgtgttccaagtgaaccatagccctttcagaaatcaagacttatattcattttacttctttgagtactctt | |
| gaattttagaaagtccattatgatcctaaggtagcaacaacatagcctattaccgtctatgatggtttacagatctattattccacgttagt | |
| tcatcactatcaactaccatgatagagttaagctaaaccattttcccaacatatgaaaaactcctattactaaagtgatacaaatggtatc | |
| aaaaatacttttttatagcaaggttcaacagtgggcccagtgcttttacactttttcaaaagtccttggagaaacagagaaaatctcacttg | |
| ccttctgtactaaaacattctaggccgaactaaaactgaaacttcatagtagaacactgtaggccaggggtgttcaatcttttaccttccc | |
| tgggccacataggaagaagaagaattgtcttgggacacacattaaatacactaacactaacaatagctgatgagcttaaaaaaaaaaaaaaa | |
| ctcatgatactttaagaaagtgtatgaatttgtgttgggcagcattcaaaaccatcctgggctgcatgtgaccctcgggcccacaggttgga | |
| caagcttgccctagctcctccatctgctgcaaagcccagcctgatacaaaaaccaacgtgataaaaagtttttgtggtgctttattttggc | |
| agtttaagttatataaacaatgggtacagtttcattttctaaatataaaatttttacattgaatatgaatttttaagacaaattatctgaa | |
| ttctgattctcatatacctaactactaatatcttctctatttgttgcccaatgagattaatccacctcttaaacacttcaccatcaagaaa | |
| aacaattttgtattttaaaatgaacccatccactttcattcagctattttatattcaggcatcatcctaaggaa | |
| agaaaggttctgacaaagattaatacagatggataagtagtagcaagaaatcaaaaactgcataaaattctagcaataaagtgttaaatt | |
| atggtacagttacattctggatcatcaggtatctgaagaatatttcatagactgttaaatgattgcattataaagtcaggtttttttaaagca | |
| agattccaaacagtaaacagtttctctctctctctctctctctctctctctctctctctctcaccaaattagttataatggtttccgcagga | |
| tgagaggggttgggaaaaagtttggtgatatttattttcttcgtttcacttttgagttttccaaagtgctatgaccatcatcagtaaaatat | |
| acatttccaaagcctttgacacacggtaacagtcctacacagtggatgaactaagagcttctctacccttagatgggtagggagggaggaaa | |
| gacaaggaaactgagttgtttaagtgtcatacacgagaacgtggctttaaggtctgggaaaacctgcgagggctgtgacgtcagactgtgaa | |
| atgcacgctatgtccattcaccaagacgttccattttaaaacccataaatccgtagctatacctgtttccaaggtgcctcgtgttaggcctc | |
| tggtcacagcacttggcgcccttcttgggatctcttctctccgcccccactaccccaccccacaagcacactctagtcccctccaatcaa | |
| tttcaggcaggtctcgccgcctccggagccacgctgggggtgcaagggccctggacccgaaagagcgcccgcccggcgacaag | |
| agatgagatgcacgctgctcctccactcctcagcccccaccatcctcctcctggatcctaacttccccactctctcaattcctagagacg | |
| ctgcggatcccagaggcttaactggcagctggaacgaggtcctccaacaagaatttagacgctaggtccaattatcactccaccgcgc | |
| gcactttccgcaggagcgatgtgatccgttatcataactgcggacctggggttccacgtggaagacgattgggatttcactggccgcg | |
| gtgggggtgggagcagacagagtctgagtggggttagtggactcgagacgaaaggcaggacatgacagaaggcaactctgggtc | |
| acctctccagcttggaactggctaggccttgttttggaggggatgggtagatgaaaagtgagtcagggttacccggaggaaccacgg | |
| ggaaagtgcgcttctgagactcttgacagccatttcgttcccttccaagccagatggagacccaagagtgttgaaaggccacgacttcc | |
| ctcagtttctccatctgggggtgcaggatggtatagagagtggcccgtagtatttttccagtgacgatgtctctccattgttttcttcttata | |
| ttgcagctttccccatgtttgaaaattttcttttcaaatgaaatcattgattagaataaaaaaaagtaagtagctattaaaacaagatcaat | |
| ttccatgacagtaagccaaccgatggagaaaaccttgggaattaataaatgaaggatttgtttggtagatgataaaaggtccttttaaagggtc | |
| tgactcttcctagaaaaacccaccaacttgggaccgcaacagatttaccatatcctaattcatgctattttaatgtgtattcagcaaaccca | |
| catgtgtttacaattgtcgaagctaccaaatgtcaatagcgttttttttctatttgttgaatgtgaatctcttgtacgaagccatataaacaga | |
| agaaattacaggaatgattttaaatcacatacaaaaccaatagtattgctagaggagagttagtcaaggacggcattatgaagaaagtga | |
| gggagaatttccaaagagcagaacgatagggcttggtggaccaaagaacgtttccatctaaagggaatggcaaatacttagagtctct | |
| gaacccactgaatcttggactatttaactaatatttgtagttccagatatagcacagtgccttgtacatagtggtatttttaaaaatatagtgc | |
| ctcgtagattttttttcaacttttatttaggaggagagggcacatgtgcaggttaattacaaaggtatattgcaccatgctgaggtttcgagt | |
| acgactgaatctgtcactcaagtagtgagcacagtacccacagtaggtagtatttcagccctcgctcattccctttctcctccatctagtag | |
| tccccaatgtctattgttctcatatttatgtccaattagcatttgttttttaaaaagggtggttgaagaaattctcagtgcttgtcagtgtct | |
| ctcagtgcattcatttaattcatgagccctggaatgatggtttcatttgggcagaactctacaatcaaaaagaagtaataaaagggaaaaaaaa | |
| gtgaaagccatcaactacaggattgaaattcccaaagcatcagaggtcctttcaaaaaatagtatgttgatttttaatttttatgacttattgg | |
| ctttgttcatgaaaatataaacatgttatcacaaaggattttttaattcaactatttctcagttttctctttcaccttcaaaataaaatatca | |
| taaattatttaaatggttgtgaaggcagtaggatttttttaagagagaaaagttttatagaggttcagaattacatgaacaaagacatgtaat | |
| ctcttaagcaaattgaaactaataaaatcgtacaatcaaggtaacgtaaataaaaaagcctctgctttcttaattgaattatgtgagtaacta | |
| gaaattttaaaagtatggcaaaggttaacaacagcattattacctgggctgcctttaaaaatacatatttctggggttcacgttcagaaaat | |
| ttgattcagatttgctgtgggtcccagaaatctgcattttaaataaacacttgaaggagatactaatacaagtggcccattgggacacaatt | |
| tgacaaatatgaccaattttactttttaaaccttatttctgcttctttatctttgaattgaggtccaggattttaggtaagattttaagttt | |
| agagtcagtttactggatcccagggaggagagtctgagtaatcagtggaggagttatttcaccaaatgaaggagaccctttattattatgtga | |
| ccctttgtatgaattggaaaagaatgtcttgtagataccacatttttacagtcagaacatagtttgagagaaaaaaatataacaagatatat | |
| ttgtgttttaaagcttacagaaccagacagaaaatttccacataagctatataagatacgttgtctttttaaaacactatatacacttctttc | |
| tgttcgtgcaggatgaatggatctctctctctctctctctctctctgtgtgtgtgtgtgtgtgtgtgtgtgtgtgtgtgtgttgtaataagg | |
| ggtttctttcattttatgatccagaccaggctcgtaataaacatgacaacctaaaattatgtaaaaaagaaaaatcaaagcacaagtgtttc | |
| acaggtttaacttatgcttatctaagatcagggcaagattgcaggaaaatgtagccataacagaataaagcatttatggacaaaatgatggg | |
| ttctttatgtctctgaaaagcacagtgatggggggggaaatatagatgaaaaatgtaagctaaaaagtaacaattataagaaaaactaaaat | |
| atcatgcctttcaaatgatcatttttctgcttttaagctaaaatttgtctaatattacaccagtgactttgctgatgtattaggaaaaagct | |
| tgttttgctttcttttctcgagtgccaccattttcttgctctcattctctttcaggctgccagatcatctgactcagcaattgtataactct | |
| ctcacccaatttaaagaaacagcagctgtctagagaacaatgactcccccagttgaacatctaattgttaaatgtccaacatcggacacttt | |
| gaattttactccatgcaatttacatgctgaatagttgaagttgaatatattatatttaacatttaatttttaaaagcttattgaaactttc | |
| ttcctaaatcacatggtaaagttattgttttcttcaaaaacaattaggaggagcttaacaataataggacacttcaacttccattatctaat | |
| ttaattatcacaatatccttatgttttcaatgtttcattttttcattttgtagatctggagactgaggctcagataggttgcatggcctacc | |
| aaaagtcattgactagtaattcatatatagttgaacttggttgcccatggagtgctataaatatgtatatggtttcagttccatctcttttag | |
| ttaactattattttgaaagtcgcttaacccctttgggcctctactatactcaagcatcagccgtataagtcacagtaaatatttattggttg | |
| aaaggaggttaacatctttcaaaaatttattttttgaccaaaataaaaccagtgaaaaattctcatatgactgtacatataaattacttatt | |
| cctaccttaatttaaaagcaataagtgggatacctattcaccagcacaggaaccacttgaagcgtgcagttgaaagattactttctttagca | |
| ttcacatgacctgtgagcagattctatttcttttgcttattagctgtcatggtaccagaatgaagtatgagaaactctcagtgctttcatgt | |
| tctcatctgtaaacctgagaccctatggtagtcccgtaataagaggtagataaaatagtatgtgtgaagagtcactgtaaacttttacacag | |
| tgtacgtttgtcagttattatagtgcctaattaaactatgcccttaagaaagcacattagttttttacagtaaatacctacttcattata | |
| atttttcagtgtagctagaaatttctaaactccactttaaaaatatacatatcataataaaaatatatttatgtattcagactcctggtat | |
| gttccaaggtgttaggtaaaatcagtgtaaatttgcatacttttaaattcacatctgtacagaagatctatatggtggcctttagggtatac | |
| ctctaagctattctagtattcataatcattaaagagatattaagcagtgtttgtgaacccctgttttctaagacaggaaatcaaggtagctt | |
| tagaaaactggaaaaaaagttattagtctatctatctaataacccagaataataatttccaaaggaatcactgaagataactggatttttaa | |
| ttccttcagaatggttgtcacagtctgaatatctgaatcaacagttttgaccaaaacaattttctaaaaattctttagtataaaaaattat | |
| gtgtgtgtgtgtctgtgtgatgaaaggaatgataggcagaaacattactgtcatccttacgacattcaaaatgcctaccttggagggtgac | |
| cttcagttatttttatgcaaatgtgaagaagttatttagaagtaggatatcaaagagtaacacaaaatacactaaatagtatgctttctta | |
| aggctaaattgacttgggggttttaaatcagtacagagtaaacatacagtatattctgttatcattgcctttttgaaaaattaattatgg | |
| aagttatcatcttaaccgtaacaacacaaaagataaaactctaccctcaacccagagactcaaaggaaaacatgagtggaaatgttaaat | |
| ctgtatgtgaaaagtgctaaaacatgaataggaagcagttacttatttaatcaaagttgattatatttcatcaagaagttgattcccttga | |
| gtggagttgaatcacatatcaggtgaagaatgtgatttggggaagaatggtctaacacaagaaaattttcttgcaatctttaataatatca | |
| gaggggagattggcttcagaactctcctaagttcaggaaaggacacagaaaattgaacataacagtaagactatagagtcccaagaaagca | |
| agctacttttaaaggatagttttttagaggggcaaaagggggacaaccattctccatttgatgagaaaagcttccatgtagatggtgcccc | |
| tgaaattagagtatcctaaaccagtgttaaacctatcagtgaaacatgaatattaaacctccactcccagtagtgaaaaccgaatacatta | |
| ttatttatctgtgactttcaacattatctcagaactctaacagcacatgcgtacatcagcagcataagcagaaatgagatattatatatgc | |
| ttgtgttagcaattaaaaaggacagcatatttgagaggggaaaatctgtcctatcaagaatgaaaaagagggaggttaggaaaagtagttt | |
| agagaaagtaaattttgcaattcctcagttttaactgtagtttctccattgtaccttccacttgaaatgcactccaagcagtggaggtggg | |
| tagcaatgaatgcagaggaaacactgaacacagtgacactctccagtgtcacttctcatgatttaatgaggggttttttttggaaattctt | |
| ctgtcataacatgggaaactttgttacaaagaagctgttttttcagagggttagaattcagaggtagcatcataccttttagaagagaat | |
| ttgcttgttgaaaccacagatacctgctagaatgtacaggaattaatgaaaaattactcaaaaggacatttattttgatgacctaaatgaa | |
| taacttcatagtaaatgtcatatatattctcaaaaaattaaaaagcaccatttattgagagcctaccgtgcacccggatttttatatatct | |
| gacattctttattcctcacagtaaccttatggggtaaattttattttccccactttgtgaggtgaggaaataaaggctcagaaagtttac | |
| ataacttattcaagcccacagagctggtaaatgagaggtcagttctatctgagtttaaagactaggcttgtcccacttgcatatgtgtca | |
| tttccaaaattatgattaaggatatggttggcatttcccgccacccacattaagtccaattaagtagctgtggccatagaaagaatggaga | |
| atggagagaggaactgacttcaacagctacagcaaacatttattagctgagtaaccatagctacatagttcctcaatatgtaccactcct | |
| ccattttgttatctataaatcaaaatggtggctttttaaaaagcagttttacaatatattcaagagccttctaccctttgaaaaactgcaa | |
| tactatttttagtagcaattagaaacaccttaaatatctgacaacagggacatcattaagtaaattataactttttccagtgggatgtgtt | |
| acagctgttaaaagtagcatttatgaagtgtttttggagaagtttggaaaatgctgtaataagttagaaaaagctcatttcaaaattgca | |
| taatattcacaatgtaaagattaagcaaagaaaaaggaagaagtatttcaaaatgttaataattattgctttgtgtggggtagtttttcatt | |
| ttctatgtgcagctaattccttaattatttttaaatatgtgagctttaatcaggaaagcaaatcattcaaaaatgaggggactgaattaag | |
| tgactttcaggggactttgcgtgtctttgagttccaaatttctatcactatgtattactactgaagaataatcatagaagcacagtagttt | |
| ctgaaaatggagagtcagtaatcttggcccaggttttgcaacttgctctaaagcagagtcctcaaagaaaaggaagcattgatgagttgtc | |
| cacaatgtactggataaattatcattaggaaaacatattgtagtagggagagtgaggacctctcaaacagaactgagaaccttaagtttg | |
| aacttttcttttccttattacttaagcactctgagcttttttttttgtctgcattatgaagaaagaataatactctctatcccatgggaca | |
| gctgtggaattataaattacacatataaaactgcttgatgcttgtcacatagctgggggttgaaaaaatgatagccattattttcttggca | |
| acttttaatgaattttttattatctctatttctttctgcctatctcctctaattatgtttattacttattttgttcctcaggatgaggtc | |
| aattctcaatatctgtgctgtacataatatacatatataccaaatatgtgcatatagtatgtacatacatacatactgtgctaatctttt | |
| agtgttctcagctgatcaaatagctacaaatagatataagtaattcgccacaagtaatttatcaacataaaaaaaatttacaaaaaagtt | |
| aaggaataattgtctccatgagctgcaaagatccctcatttcacaagagtacaccctagagatattttaatagtaaatttctcacatagat | |
| ttaaaatcacatttgttttgcacataatttagaaaagatacctgctatataataagtaatatacttttaagtttccttcaaaatattctt | |
| gggaagatgataataggtactgctaattctatacccagttaacattttggaaactaaggttgaaaattgtgacttaactataattatgcat | |
| taaatctacaacacatcaaagaattttgcattttgtactccttactaagatccagtttgagtaggaagataaattttacagtaattctgaa | |
| tgagggaagttggcacagagtttctaaaagagtaccttccttatagcaaatactaaataattgtgctatattgaatttaattaaatagaga | |
| atagtaaaagggagaaagaaacatccaatgttttgaaacttctagagatctactcccagggacacattgttttttcttagcaaatctgtt | |
| ctggaggtctgctctactttctcagaggtctccctttcatgctgaagctatcttttttcttgtggaacataagtaattaaataccttgca | |
| attatttacctaagaaagtgtttctttcccgtttaaaatgctcttaccacccacattggactcgattatcagaatttttatccggggcag | |
| cttcaggagcactttggcacttcggggctaaaccacaatctgtttttacatgtttgtgattatacccgttttgtagatcaagacattgaa | |
| ggctagtaaaaaaaaaaaaaagtcattttttcagggtaacaaagtaggtggtagaactaggacaggactctaatttccttacattattgc | |
| ttttctaaattaaagggatgcatggaattattcctccattgcctttgccttcaaataattatctattgcacccaacatcctattctagaa | |
| ctcatctatgaaggcttaacacagctgtacctgggagctccattacagggcatatatctcgctctcataagctacttcctaaggaattct | |
| ctttaattatgggagcttttccagactctgaaatctttttttcctggtaacacaagtgtgaggtgtcatttatcagaatgcatcacccca | |
| gtcttccctcctcaaatgattactgtaggctccactcaagagctcatcccagttcaagaccaccttcctcctccagagaagcaaatatat | |
| atatacacgtatatatatatatacacgtatatatatatatacacgtatatatatatatacacgtatatatatatatacacgtatatatat | |
| atatacacgtatatatatacacgtatatatatatatacacgtatatatatatacacgtatatatatatatacacgtatatatatatacac | |
| gtatatatatatatacacgtatatatatatacgtgtatatatatatatacattttttttttttgagacggagtctcgctctgttgcccagg | |
| ctggagtgcagtggcgcgatctcggctcactgcaagctccgccccccgggttcacgccattctccttcctcagcctccggagtagctggg | |
| actacaggtgcccgccacctcgcctggctaattttttgtatctttagtagagatggggtttcaccgtgttacctaggatggtctagatct | |
| cctgacctcgtgatccgcccgcctcggcctcccaaagtgctgggattacaggcgtgagccaccgcgcctggcagagaagcaaatatattga | |
| tggttgttaccaatacatgctcttgactaagaaaccttctttcttaattaatattgacaactttaagccgagtgcctgacatatattagg | |
| tactcagttactctttttcaactaaagttatgaatgatgattctaataaaagtaacttatttgtctactagttttattatgtttatttaa | |
| ttcattagaaaggccatggacatagtacaaaattcaaacaatataaatcatggaatgtgaaaagtaagtcacatgcccatcccagttctt | |
| catttccttacctcacaggtaacagcttttcctgtatctccccagagatattctatgtatattttgtttttaacaccaagctatatttaa | |
| aacaattatctttaataataatgttaatattgaaactggtaaagaaatatgtgtgtattatctcacctcaagcgtaaacaatagaacaag | |
| agagagcccattttgaaaattatggacaatgaatctagaaataatctcaaaagattttgcagtcaaaaaatagttcattagatacatgag | |
| aactgtcacttggtctcagtgtagagctattgcctcaactccctttattttcctaacaaaatcatcttgcttatcccatgaaatacgtgca | |
| tattgccaatcctacaatgccgcatcagaaccagaacccaactctggaacactaccttctcaagtatctttctgtctctttatggtaata | |
| tgttgaattaatattcacatctattatgactagtctttgatttgtagggttgctgaagtagtagcaccactgcagggctttctttagttt | |
| aaagaaagtaatcaggtgtccctactgtgtcatgatctccaccctcagctgggttctccagtctggttttaaagaacaaaacaaaaggct | |
| tctctgtctgagtcttactcaacccatcctctctactcataagaggtattccaaacctttacgattctcaaacttcctaaccgaccatct | |
| tattttcactctgcaaacaagctaacctcctcattcatagaaggaagtgcctcaacttcctccccgttctgaccttttctccctcccaaa | |
| tctatgtatctcttgtgacaaaatctataaccaccgctgtactttgagttctatttcttcattatttttgagggacctcaagtcctcaaaa | |
| aatatcctatcttgcctgtgtacttaacttttcttttattcttttctaactttcccttctcttccttggcacttgcccttccaggtatatg | |
| tgtgctcaggtctcctccaccttccatctgcctcacttcatggcatagggccttgaactatcacaaccaagctatgaaagagtagtcaac | |
| gcagtgtccccacttccttgccatcccattatcctagtttttcttttggctctctgaggagtccttcacaggctggttttcaggaataagt | |
| ctaaatgaatcactttcagttttcctaaacttctatgcctttgcacatcctcttacctctgcctagaatatctttctccttcttttccat | |
| ctttaaactctcacatcattcttcaagactgggatcagctctcagcatccggaagcctttgcctactagagacaaatgagaatgagtttg | |
| gtcaccttttcattttcttgtatcattctgtgctttattttgctcttctaagagcgttacatgcttcatttaatccctaaacaactgttt | |
| gaggcaagtacagttattatcctaatcatgcaaatgagaaaacagaggcccagacatgttgagtaactttgataaaagttaaa | |
| gaaccaataagtggaacagttgaggtttgaaccctggcagtctgactgtagagatactatgtttgacctactcccctctgcccccacccc | |
| atgtctgcccttagtttctgagcttgttgaatgaatgaacaggtggtagtctttttttgttataagactgatcagaattaagacaggtttaaa | |
| tttcacgtgtagaattttcaaaactgcaaaggcagtgcaaatctaaaaaaagaatggcattctcaggaaagaggaaaagtaagtgtgaga | |
| ataataataacaataaccaacaaactttagtaaatttagtaaatgtagtaaatttttacattaaaagcttttggacatacattatcatatttt | |
| atggccacatgaaatatattataatcccattttgcacataggaaatctgagactggcataaggagcacagagatccaggactttatatttt | |
| cattcttctaggattttgcacctcaggtcgatatgtatgagtaaactgggagtataatgggctctttaacagaaaaactaggaaagttttc | |
| ccactattattaattatttacataatatttttttaattttattattatttatactttaagttttagagtacatgtgcacaatgtgcaggttt | |
| gttacatatgtatacatgtgccatgttggtgtgctgcacccatcaactcatcatttagcattaggtatatctcctaatgctatccctcccc | |
| cctcccccctacataagatttataatggataatggacttcaatttctagagcaaaatggccccacccaaggatgccataatccttccagag | |
| ctctactgcaagatatgagatatacatatctaaaacttgttcttggtatttccaaagcagtcaacttttacacctgtttataatgcatcca | |
| aatgttgtttttatatggttgcatctcccatcttcttcaccaatagctatatatatttttcacaagagctgaaagagttcttgatgtaggaa | |
| tccatggtagagtttcagagaaatccctgaattcactgaaagttttatctagaaatacatgtgcaagtgaacacatcttttttaaaaaaaa | |
| tcattacctactttcttttttgagaagaaggtatttatttcaacagactcttgaaggagcctactcttcccactctcccacccccattaaga | |
| accactgtaggccgggcacgatggctcatgcctgtaatcccagcactttgggaggctaaggtgggtggatcacctgaggtcaggagttcgag | |
| acaagcctagccaacatagtgaaaccccgtctctactaataatacaaaaattagctgggtatggcagcatgtgcctgtaatcccagctact | |
| gcgggaggctgaggcaggaaattgctcgaacccgggaggcggaggttgcagtgaaccgagagagatcgtgcggtgccatttcactccagc | |
| ctgggcaacagagcgaaactccatctcaaaaaaacacacaaaacaaacaaacaaaaagaaagaaccattgtattagtgatggaaatgtgt | |
| tccctccctcccatcctggcaaccactttcttcctcctccatcataaaatatcttaaactaaactaaaataattttatttatcgatagt | |
| ttgaattttccctatcattgctacacagctaattgagaggtaccccgaggaaaatataaatggtacagtaatgcattgtagattttaataac | |
| atacttgacatcccaaattgttttcattggcttcattttaaaaactacatgttttaaaatcaagcagacactaaaagtacaagatatactg | |
| ggtctacaaggtttaagtcaaccagggattgaaatataacttttaaacagagctggattatccagtaggcagattaagcatgtgcttaagg | |
| acatcagcaaagtctgagcatccattttttaaaacgtagtacatgtttttgataagcttaaaaagtagtagtcacaggaaaaattagaact | |
| tttacctccttgcgcttgttatactctttagtgctgtttaacttttctttgtaagtgagggtggtggagggtgcccataatcttttcaggg | |
| agtaagttcttcttggtctttctttctttctttctttctttttttcttgagaccaagtttcgctcttgtctcccaggctggagtgcaatgg | |
| cgcgatctcggctcactgcaacctccgccttctcctgggttcaagcgattctcctacatcagcctccgagtagctgggattacaggcatgcg | |
| ccaccaagccccgctaattttgtattttttagtagagacagggtttcgccatgttggtcaggcttgtctcgaactcctggcctcaggtgat | |
| ccgcctgtctcggcctcccagaatgctgggattatagacgtgagccaccgcatccggactttccttttatgtaatagtgataattctatc | |
| caaagcatttttttttttttttttgagtcggagtctcattctgtcacccaggctggagggtggtggcgcgatctcggcttactgcaacct | |
| ctgcctcccgggttcaagcgattctcctgcctcagcctcctgagtagctggaattacacacgtgcgccaccatggccagctaatttttgta | |
| tttttagtagagacggggtgtcaccattttggccaagctggcctcgaactcctgacctcaggtgatctgcccgcctcggcttcccaaagt | |
| gctgggattacaggtgtgagccaccgcgtcctgctccaaagcattttctttctatgcctcaaaacaagattgcaagccagtcctcaaagcg | |
| gataattcaagagctaacaggtattagcttaggatgtgtggcactgttcttaaggcttatatgtattaatacatcatttaaactcacaaca | |
| acccctataaagcagggggcactcatattcccttccccctttataattacgaaaaatgcaaggtattttcagtaggaaagagaaatgtgag | |
| aagtgtgaaggagacaggacagtatttgaagctggtctttggatcactgtgcaactctgcttctagaacactgagcactttttctggtctag | |
| gaattatgactttgagaatggagtccgtccttccaatgactccctccccattttcctatctgcctacaggcagaattctcccccgtccgtat | |
| taaataaacctcatcttttcagagtctgctcttataccaggcaatgtacacgtctgagaaacccttgccccagacagccgttttacacgca | |
| ggaggggaaggggaggggaaggagagagcagtccgactctccaaaaggaatcctttgaactagggtttctgacttagtgaaccccgcgctcc | |
| tgaaaatcaagggttgagggggtagggggacactttctagtcgtacaggtgatttcgattctcggtggggctctcacaactaggaaagaat | |
| agttttgctttttcttatgattaaaagaagaagccatactttccctatgacaccaaacaccccgattcaatttggcagttaggaaggttgt | |
| atcgcggaggaaggaaacggggcgggggcggatttctttttaacagagtgaacgcactcaaacacgcctttgctggcaggcgggggagcg | |
| cggctgggagcagggaggccggagggcggtgtggggggcaggtggggaggagcccagtcctccttccttgccaacgctggctctggcgagg | |
| gctgcttccggctggtgcccccgggggagacccaacctggggcgacttcaggggtgccacattcgctaagtgctcggagttaatagcacct | |
| cctccgagcactcgctcacggcgtccccttgcctggaaagataccgcggtccctccagaggatttgagggacagggtcggagggggctctt | |
| ccgccagcaccggaggaagaaagaggaggggctggctggtcaccagagggtggggcggaccgcgtgcgctcggcggctgcggaga | |
| gggggagagcaggcagcgggcggcggggagcagcatggagccggcggcggggagcagcatggagccttcggctgactggct | |
| ggccacggccgcggcccggggtcgggtagaggaggtgcgggcgctgctggaggcgggggcgctgcccaacgcaccgaatagt | |
| tacggtcggaggccgatccaggtgggtagagggtctgcagcgggagcaggggatggcgggcgactctggaggacgaagtttgca | |
| ggggaattggaatcaggtagcgcttcgattctccggaaaaaggggaggcttcctggggagttttcagaaggggtttgtaatcacagac | |
| ctcctcctggcgacgccctgggggcttgggaagccaaggaagaggaatgaggagccacgcgcgtacagatctctcgaatgctgag | |
| aagatctgaaggggggaacatatttgtattagatggaagtatgctctttatcagatacaaaatttacgaacgtttgggataaaaagggagt | |
| cttaaagaaatgtaagatgtgctgggactacttagcctccaattcacagatacctggatggagcttatctttcttactaggagggattatca | |
| gtggaaatctgtggtgtatgttggaataaatatcgaatataaattttgatcgaaattattcagaagcggccgggcgcggtgcctcacgcc | |
| ttgtaatcccttcactttgggagatcaaggcggggggaatcacctgaggtcgggagttcgagaccagcctggccaacaggtgaaacc | |
| tcgcctctactaaaaatacaaaaagtagccgggggtggtggcaggcgcctgtaatcccagctactcgggaggttgaggcaggagaa | |
| tcgcttgaacccgggaggctgaggttgtagtgaacagcgagatggagccacttcactccagcctgggtgacagagtgagactttgtc | |
| gaaagaaagaaagagagaaagagagagagaaaaattattcagaagcaactacatattgtgtttatttttaactgagtagggcaaataaa | |
| tatatgtttgctgtaggaacttaggaaataatgagccacattcatgtgatcattccagaggtaatatgtagttaccattttgggaatatctgct | |
| aacatttttgctcttttactatctttagcttacttgatatagtttatttgtgataagagttttcaattcctcatttttgaacagaggtgtttc | |
| tcctctccctactcctgttttgtgagggagttaggggaggatttaaaagtaattaatacatgggtaacttagcatctctaaaattttgccaac | |
| agcttgaacccgggagtttggctttgtagtcctacaatatcttagaagagaccttatttgtttaaaaacaaaaaggaaaaagaaaagtggat | |
| agttttgacaatttttaatggagacgggagaagaacatgtagaaaaggggaaatgatgttggcttagaatcctaactacattggtgtttaa | |
| tataggaacatttatttatataacattttaaagtactaaattcatattagtatattatcaaatggatatattatcaaatgggtttaagcatcc | |
| tacacattttaattcaattgattcattttctttttgctttggatttctatcatgatttaaatatttacatatgggttactttttagatttttc | |
| atactatgaaatataagaaaaacctttaaggctagttttatgaccaagacgaaggacttcattgaatacacaaaacaataaatatactgca | |
| acattttgtctttctttttgtagctgcaatttggtttgcttatactttctctttgtctctttgaaaactgagtcagtttcactttctcagga | |
| caggatttaataaccataatataatttagtataattccttgatttaggcaaattatgcaatttgtgtttagtatgaaatgtacctaaaaata | |
| agtaactcctctttaacaccaccatcctcaaactaatataacaaataacagttatcctaaaataaattgtctacttccaccatgcagcact | |
| caaattttaaggttgctatgactgcagacagtattttaaaattcctctctggaaatggctttgtttccaagatgatttaggaaccaaagag | |
| gtgaccatctcttgtttaatgaactctcaaatcataaacctgggaagtgttttagtttcctactgctgctgttacaaattatcacaaatgt | |
| gttagctaaaacaaacacaaaattattattttacagttctagagatcagaagtcaaaaatgggtccacaaggtttcattccttttggaaac | |
| tctaaggggcaatctgtttccttgtcttttccagcttctagtgaccatcaaattccttggctcatggtctctgtattttctctgtggcct | |
| gtgcttccattcttgtatcttctctctgactgtgaccctctaataaaaacacttggggttatgttgggcccaccctgaaaattctggataat | |
| ctccctcaagaccattaattaaatcacatctgcaaagcctcttttgccacataagttaatgtattaaaagtttttgaggattaggacatag | |
| acattgggggtgggggggcattattcagcctaccacaggaaggaattttagggttaattaaactagccttcttattttatacttgaagaa | |
| attgaagttttggaattggagagcattatgctaaatgaaataagccaaacacagaaagacaaatatcacatgttctcacttatctgtgaaat | |
| ataaaacaattacattcttagcagtaaagagtagaatggtggttactagagctggggggtgggaggaatggggagatggtaatcaagatata | |
| aagcctcagttaagatgggaggaataagtttgattgttttttttgagatgtgtttcatagcatgatgaatatagctaaatagtaaatccc | |
| aaatgctctcatttgacaaaaatgtcaaatatttgagatgatggataggttacttagcttgacttaataattccccattgtgttcaaaga | |
| tcataacttcatattgtaccacataaatatatacaactgtactatcccaatatataattttaaaactaatataatgaaaaagaaattgaa | |
| gttcaacattcccagaagctaagtgtaacttaaaagttttgtgagaatttgttttaacaaacaaacaagttttctctttttaacaattac | |
| cacattctgcgcttggatatacagcagtgaacaaaaaaaaaaaaaaaaatctccaggcctaacataatttcaggaagaaatttcagtagt | |
| tgtatctcaggggaaatacaggaagttagcctggagtaaaagtcagtctgtccctgcccctttgctattttgcccgtgcctcacagtgctc | |
| tctgcctgtgacgacagctccgcagaagttcggaggatataatggaattcattgtgtactgaagaatggatagagaactcaagaaggaaa | |
| ttggaaactggaagcaaatgtaggggtaattagacacctggggcttgtgtgggggtctgcttggcggtgagggggctctacacaagcttc | |
| ctttccgtcatgccggcccccaccctggctctgaccattctgttctctctggcaggtcatgatgatgggcagcgcccgagtggcggagct | |
| gctgctgctccacggcgcggagcccaactgcgccgaccccgccactctcacccgacccgtgcacgacgctgcccgggagggcttcctgga | |
| cacgctggtggtgctgcaccgggccggggcgcggctggacgtgcgcgatgcctggggccgtctgcccgtggacctggctgaggagctggg | |
| ccatcgcgatgtcgcacggtacctgcgcgcggctgcggggggcaccagaggcagtaaccatgcccgcatagatgccgcggaaggtccctc | |
| aggtgaggactgatgatctgagaatttgtaccctgagagcttccaaagctcagagcattcattttccagcacagaaagttcagcccggga | |
| gaccagtctccggtcttgcctcagctcacgcgccaatcggtgggacggcctgagtctccctatcgccctgccccgccagggcggcaaatg | |
| ggaaataatcccgaaatggacttgcgcacgtgaaagcccattttgtacattatacttcccaaagcataccaccacccaaacacctaccct | |
| ctgctagttcaaggcctagactgcggagcaatgaagactcaagaggctagaggtctagtgccccctcttcctccaaactagggccagtt | |
| gcatccacttaccaggtctgtttcctcatttgcataccaagctggctggaccaacctcaggatttccaaacccaattgtgcgtggcatca | |
| tctggagatctctcgatctcggctcttctgcacaactcaactaatctgaccctcctcagctaatctgaccctccgctttatgcggtagagt | |
| tttccagagctgccccagggggttctggggacatcaggaccaagacttcgctgaccctggcagtctgtgcaccggagttggctcctttcc | |
| ctcttaaacttgtgcaagagatcgctgagcgatgaaggtagaattatggtcctccttgcccttgcctttcctttttgtgatctcaaagcat | |
| cctccctccgcccccattccatggccccagttccctactcccacagctgtctgctgaaactgccaacattactcaattgtttctgggggga | |
| ggaacatttttttttgaaacaaaatagatatatgaaacagtacacgggaattaacacgaatatttaaggtaaaacatgaccttgaagatt | |
| atgaaatccatcttattttggcccagaacgggggcattgggctccttgggccataggggagctggggaggacagggtgaagagttagctct | |
| aagccctctgcttggagatgctgtaaatacagaacgcaaaatcaccttcgaagttaaagacgcgaagttcttctttactcggcccctcct | |
| cccctcccccccgccaattccctccagttacagctagcatccaggtcccgggaggtgaagaaggagacttcggctccagttacagctagca | |
| tccgggtcccgatttagaaggagctgccaattacagcgcggttccagggctgagcaaaaagcctgaggagccaagtgggagagggagtaaa | |
| actactgaattgggccacaagcaaatgaataaactgaacgactcttaaccaaacctaatatatttaatccaaacacacaagtctttcatt | |
| tcttccctcctcccttccttctcttactccccaacaccccctcttcaagcacaattaattatatggttagattctactgcgtgatcagcc | |
| ctgttctaggtggtgggcacgccaaggtgaatgagaccaaacaagagtcttgccctcatggggtttacatttggagacagagtcgatctgt | |
| gtgcccaacctggagtgcagtgcgcgatcacagctcactgcagcctcaaactccctggctcaaggggttctcccacctgagcctcccgact | |
| agctgggaccacaggtgcacgccacgacgcctgggtttgtttgtttgtttaatagagacgaaggtctcaccatgttatctgggctcaagcga | |
| tcatcccccctcctcctcctaaagtactgggattacagtcccaagctatcttgcccgacctgggaaacagacgttaaggaagataacaatc | |
| tattttcagagagcgagtttataaaaccaatgcaatgggtaaatatgaagtgtgaataggaggagaagctaaagagtggtcggagaatcta | |
| atgcaagctacgggagaaagaaactcaagtgcaaatgctgcctcaggaataaacgtaaaaagagactttcaagtgcaaatgctccctcagg | |
| aataaaataatcttgagactctcaagtgtaaatgctgcctcgggagaaccgaacggcgagctggagcccatacgcaacgagattagagagga | |
| aggcagaagccagagcacatgaataaatgagcatccattttgtttcagaaatgatcggaaaccatttgtgggtttgtagaagcaggcat | |
| gcgtagggaagctacgggattccgccgaggagcgccagagcctgaggcgccctttggttatcgcaagctggctggctcactccgca | |
| ccaggtgcaaaagatgcctggggatgcgggaagggaaaggccacatcttcacgccttcgcgcctggcattgtgagcaaccactgag | |
| actcattatataacactcgttttcttcttgcaaccctgcgggccgcgcggtcgcgctttctctgccctccgccgggtggacctggagcgc | |
| ttgagcggtcggcgcgcctggagcagccaggcgggcagtggactagctgctggaccagggaggtgtgggagagcggtggcggc | |
| gggtacatgcacgtgaagccattgcgagaactttatccataagtatttcaatgccggtagggacggcaagagaggagggcgggatgt | |
| gccacacatctttgacctcaggtttctaacgcctgttttctttctgccctctgcagacatccccgattgaaagaaccagagaggctctgag | |
| aaacctcgggaaacttagatcatcagtcaccgaaggtcctacagggccacaactgcccccgccacaacccaccccgctttcgtagttt | |
| tcatttagaaaatagagcttttaaaaatgtcctgccttttaacgtagatatatgccttcccccactaccgtaaatgtccatttatatcatt | |
| ttttatatattcttataaaaatgtaaaaaagaaaaacaccgcttctgccttttcactgtgttggagttttctggagtgagcactcacgccc | |
| taagcgcacattcatgtgggcatttcttgcgagcctcgcagcctccggaagctgtcgacttcatgacaagcattttgtgaactagggaagc | |
| tcaggggggttactggcttctcttgagtcacactgctagcaaatggcagaaccaaagctcaaataaaaataaaataattttcattcattc | |
| actcatttattgtcaacatttattgagcacctattacaacaatttcatcgcatggaagacagcatcgtttctgacactgttgtttcatgta | |
| tctcttagaaaaacgctgctattagacatctaacactatttatcttgaggtgataaaatatcaaaagccgtgtctcaagatcgatgaaatgc | |
| ggttaaaatgatgaatagaaactctagggggacctcatatcgatagactcgagactggcacatctggagatccgtatttatccggcttccc | |
| cttccagatcacgcgaggtttgggatattttgctcaccaggcctcagccaggtaactgaatccagccaaccctggcccatagtctcggaatc | |
| cgactcggctcccagtccccgcctcggcgttctgagacccccaggctggggttccagagggctgtgaggttgcgaatgactgctgccaaac | |
| cggaaggaactctgcggttctctgccacagtgggattgttgcaggcacgcggctcagacttcactgaggttgggagatgctcctgtcc | |
| acgctgcctcatcccgtgctggagcactgcacctctatttttttttttagggtacacgc | |
| Protein sequence: | SEQ ID |
| MEPAAGSSMEPSADWLATAAARGRVEEVRALLEAGALPNAPNSYGRRPIQVMMM | NO: 348 |
| GSARVAELLLLHGAEPNCADPATLTRPVHDAAREGFLDTLVVLHRAGARLDVRDA | |
| WGRLPVDLAEELGHRDVARYLRAAAGGTRGSNHARIDAAEGPSEMIGNHLWVCRS | |
| RHA | |
| NOTCH1 | SEQ ID |
| cctcgcggtcaagtgctcgggcaatcacggccagggatgtctggcggcgatcgatcctctggacgcctaaagccgcggccacggg | NO: 309 |
| gccctcgggagggagtgaagccgctgggctaggggcgcacacacggctaggccactctcccagagccctgccccgggcccggg | |
| gtcccccaacgtggcctcagctgctccccgcccggcccagcgcacggtgcacacggctgtccgcggcctcgccctccccattccgc | |
| cccgggctcctccgcttattcacatgcaaatttcagtcgccagttgtcgccgagcgcggcaaccgccagagccggatccttccgcag | |
| ccccggctcaaacttttggcctctgaaaactttcaaacgagaagtagtcccaggcgcccgctcccgacccacgccgcgccgccggg | |
| tccctcctccccggagaggctgggctcgggacgcgcggctcagctcggggaggcgcaaaggcggacggggcgtgcgggagga | |
| ggtggccgcggagggggcggggggaccggcgggggtggggccgggcggggcggggccggcgggggcggagcgcacctc | |
| gactctgagcctcactagtgcctcggccgcgggagggagcgcaagggcgcggggcgcggggcgcgggcgcgggcgcgagcg | |
| cagcgaaggaacgagccgggcgcggagccgggcccgggggcctgcgagagcacagcgccgccagccagccggggaagaga | |
| gggcgggaccgtccgccgccgccccgggaccgtacgccgcgcgtgtgcgtcccagccccgccggccagcgcaggaggccgcc | |
| gcccgggcgcagagggcagccggtggggaggcatgccgccgctcctggcgcccctgctctgcctggcgctgctgcccgcgctcg | |
| ccgcacgaggtaggcgcccacccacccgcgagcccccactttccgcgccctttggaaactttggcggcgcccggcgcgcgcgcc | |
| ccacggctgggagcgggcggcggggaggccagcatggagagggaaaagcgggcggcccggggcgtggggttctggagtccc | |
| gggatcagggaggaccgaccttccccctcgatccccccgtggaggcggactcgcgccgcccgtgcctggagccgagttaggagg | |
| ccggtgtggggtgctggggccccggaggccctactccgggcccgcccttcacccgccgcgcgtggggcttgccgccggtcggcc | |
| gggcgggcgggctgcctactatttttcgatttgaatagagtcggttttggtttcctgttgcttctccgggccatttatcttctttcttctt | |
| cgcctctggcccacgccggggcggatgttggggcgcggagtgtgggctctgcggcgccgcgttcgccttcactgacccgcgcggccggg | |
| ctgggtccccgggctcccggtcgccccgcccgccggtgccccccagcccggctctcagtttgggggaggggttgcgtaagaagcc | |
| gccgcgcccggggggactgaactttccttttgctttgcggagttgaagtttggaaagcttgggggcggagagcgggacgcgggtgg | |
| ggggctcttacatttctccccgccgcacagcgagcggggtctctggggaatcgagtgattaatccactctttctccgagagttggaggc | |
| gagaattatctgtcctcttccagaaagtgcggctctgtgtcacacccccctcccccgtttctcagccccggtaagatggggagggagg | |
| ggcttgagtaattgatcccttctcgagatggggtcgaattccttccgaatgggggaccttcatccccctcctgtgggtgtatgggggctg | |
| ccctggagatgcgcgcccgcggaggcaggtattgggtgtcggcggaggcggggccgcgtccccagggtgctgtcccggtgcccc | |
| tggaggcggccccgactccacaatgggccgctctgattctgaggcggaggccggcgctttgttggcgggcggggtagcgggcga | |
| gcagctgtcgcattttcccacgggcgggagctgagtgtggccaccccccctccccccgtccagctctgcccccttgcagaacgggttt | |
| gaacagaggcaatctcggcggctggatggggggccctgccctgccagactcctcagtgagcctccagggtggggggcaacgcttt | |
| ggagagtagcccactttgtctctgcttttcccccactgcgtccaggcggcacatcaggccaccccaccctgcttcaagcaggacgtgtt | |
| ccctgctgctccccttccccccatttctgactacagaactctgggcagaatgttgacgcccctttggtttcatggaggggcttcctgcgg | |
| acccgtgccccagcaacccatgatactgagcagagtgcgtccggggtagggggcggacgatgccccttggctgggtgcaggtccc | |
| cgcccccagggtaaccagggccttcggtggtggtggtgcgggtgagactgacctctcttctcctgcccctgcctaggcccgcgatgc | |
| tcccagcccggtgagacctgcctgaatggcgggaagtgtgaagcggccaatggcacggaggcctgcgtgtgagtaccacccctgc | |
| gggacctgttgctttgtcgagggcagagcccctgccttctgcagccgcgcaggggacagaacactaggccattgtcttctggagatg | |
| gcccagagtctgggcacggtcacgtgctgacttttattggacaaagtctggcaattacttaatcaccagcaattatgctgcgtgtggagc | |
| tggtctggctgctgagggcttgggcactcctccgtgggccacctcaggctccggggtcactaagtgggagtcccagtaccccaattct | |
| tctttaagtccccaagaaacagttgatctgccaggggaagctctggaccatttactgagaggcccagcccccaccaccctgccaactt | |
| ggggcgcctcctctgggcgggtgatgcctcctgggcacaggtgatgcctcctctcgggggtgatgcctcctaggtgggtggtgatgc | |
| ctcttgggctggtagtgatgcctcctctggcacaggtgatgccacctcctgggcgggtgatgcctcctctgggtgcgtgatgcctcctg | |
| ggctggcggcaatgcctcctctgggcacaggtgatgccacctcctgggcgggtgatgcctcctctgggtgcgtgatgcctcctctgg | |
| gcacgggtgatgccacctcctgggtgggtgatgcctcctctgggtggtgatacctcctctaggtgggtgatgcctcctctgggtggtga | |
| tacctcctctaggcgggtgatgcctcctctaggcacaggtgatacttcctgggcaggtgatgccacctccgggtgggtgatgcctcttc | |
| aaaggaaattgactcttcaaaatcggacccgcctttgggctctctgtttgccccagctccctcttcccggcctcaggctggggctgggg | |
| aagagaaggtggaactcatgttttggcgtggggtgtggggtaattattgagtctggtttctgtcctggcatctgcaaactctgacctcaaa | |
| atcccgtggcccttctggtctgccagcctcagatgtaaaatgggcactgagggggccgggaagtcgtgcggaaccgctccctcctgc | |
| ctgcctgtgagtgcccatcagccgtggggaactgttctgctgtggccgatgcccttcaacttgagtccatccctgatgagaataatgag | |
| gaggtgtttggtgtctggaaaaagagttccctccgccccccaggagctacagatgaagaaatggctccccagccagtctgcgcgccc | |
| tcattccaactgcagaaggtcccattcccttgacaaccccagcccggctcctgcctgtgccgactgcccagcctgtgggcagcaggc | |
| cccctccctccccggcccccaccctccccaaactgaagccaattaagcaggtctgtgagcagtttaatggacaaagccgattgtgtctt | |
| tgtcagacactaatgaatgcgcactacttttttttttttttttttttttgctttttggggcccccttctccctctgcagaagccgcacggac | |
| gcttgatgccctaaatcttgtttcctttcattcagagaccgacagctgggatccaggggaagtgcagcagctattgttgggggaggcggattaa | |
| tgccgtgtgattaattatgcgtggcttcccagaatgtataacccccttccccgccactgcgctcccggctcggactttgcctctcacccat | |
| tcggggctctgggagaagcttcttgctcctgcagaggactttgggagggtgggaggaacaccgtgctgggaaaattgggggcctgtt | |
| gcttcttctcagtggcaccagggagtaggcacagcctggcccacaggggtctgggactgaggtctccctgggccagagcgctgtgc | |
| tttggggtacagattgctgcagctccctggtgtctgggttaaaaccctgccacagggccgcaaaacactggtgtcatgggcccactttc | |
| ccattccccaaacctgggatcccggtgagctgtttccagcccaaggcaggccgccgaccatggctttggtgcctggccttgtccccttt | |
| gctggggcctggcgccccctccttcttggcgggaagcctgcttcagtttggcctgacaggcagccgagctcagcccacagagaccc | |
| cattgtgcacaccccacaagtccaacgagaagatgggacggaagcgcgtcctcaggctcctgcttctgggccgcagagttggaggc | |
| cagggccacagccgagcccggcttgtggaggcacaggtcagggctcacacagcagtctgggggacattgcagccgcttcggaga | |
| aggccccgggctccctccctgggcttgagccccacccaacatacacccctcaggtgcctctgccttcccgttcctgcccatccttctca | |
| gaaggcccaggaggtctgtccaggagttagccagggcacctgcaggaggcctggctcccccagcaaggggagaaacccacactg | |
| tttctcgaaagagtctggggtgcaggctcttggagccctgccagcctgagtgggaatcctggccccgtggtatccagctgtgaccccg | |
| gaaagagctttggcttttggtgccatttgctgcctccgctgtagaaggggcatggtcatgatgggcgtcacagtgcctggctcactagta | |
| ctgggtgaatcatttctttccatcccaaagagaaggaaccttcaccggaagctgccaatcaggtggttgcagggcccgggattggaaa | |
| aagtggttttgttggtgcaaaatgccttcccaggaggtcagagtgtcgccgtggcctggtcagccagtggccaggtagaaagcacctg | |
| gcagcacatgctggtgggcggaggatggttggtctctgggcagctgctgtgcgaggtgtggccgtggtggctgtggtcggagatgg | |
| tggccttctgagcaggcccagggggtcactgtggcccttggctcttctctagccccctcatcagctccgtcagtgtgagcaaggggta | |
| ctgaagagggctccaccttttttctctttgttttgaggagagctgggaagagctgcagggaccctatctgtctgggtgagactacgtcctt | |
| ggtctgaaacgctctcccagggctgcctgcgtctgggctgggcgttgaggcagggggcagctaagatgttggctgctgacggcaga | |
| gtcagaatggcacggggcgtcttccaggagctgggagaaatgggggtaggctgtcacacccagcacctccaggaggatgtccagc | |
| tgcctgctgggtggtgcgctttagctttggacaacctcgtgtctgaggcctggcctggccactagctctgtgaccttgggtcatcaggtc | |
| atgcaagcctcagtttccccagcatgtcagccgggctgatacacgctggatctcttggtggtgtgtgaggattgcatgagaaccgtgtg | |
| gagagggcacctggtcagcgctcctttcttccccgccttctccccgttggctgacagtgctggtttccagagcctccttcctgaatgttta | |
| attgattgacgagtgaattcagagaatcctaaaggtgctgcctggtgggctgggcctggcactgcctggggagggacttgccggctct | |
| ggggaagtccatctcccaggatggctccagctccggggaagtccatctcccagggtggctccagttcacagacacgtttgagcgcct | |
| gctgtgtgctgcgttctgctccaggccaggcccccggtggggacagggcagacatggcccctgctcctgtggagttcacttcccagc | |
| aaggcgtgagagaggcacctagctcacatgtgggacatgttgggggtgtgccggcccggtggtggcctggcatagctcccggcag | |
| ccccgtaaagctgtctggatcaggcaccgataagtgagaagaagagacccagagaagtcgccatcagccccagggtcacacagca | |
| gtggcagaattcctactagccctgcccctctccttctcccaagcgaatgtccctaaacacagccccagccagcctgagctgccccgtc | |
| atttcccgactacaagcggactgggggcgtggcttccccttaaaagaagaggaaggaggctcaggcgggaagtgacttggccctgc | |
| agccggcctgggaggctggggagggacggggtagctcctgtcacccggtctggctctttccattgagtcacctgcctcgtcttgggc | |
| gtggccaggggaggaacaggttgattctcctcctcatgctgagctgagcggaaaggcctgtgacaggacctcctgtttatgcagaac | |
| ctggtcttcaagggcgccagggttagaaaaacatgtttaaaaacatgcagcatccgaggtgggcagatcacctgaggtcaggagttc | |
| aagaccagcctggccaacatggcgaaacccccgtctctactaaaaatacaaaaattacccaggcgcagtggcgtgtgcctgtaatcc | |
| cagctacttgggaagctgaggcaggaggatcctttgaacccaggagacggaggctgcagtgagctgagatcgtgccactgcactcc | |
| agcctgggtgacaaagtgagactctgtctcaaaaaaacaaaaaacaacaaaaaaaaccccacacagtgtcaaatagaaagtctcacc | |
| tactcagactggggagacatcaggaacaggaaggcctggggcggagactggggtgggagcctggagtgctctgaaggaagctgg | |
| ctccttggggcaggccctgcctctgaacagcagccaacatgttttccattgaaaagaataataatagaaaaagagagagggagaaag | |
| aaaacagcaaccctggcaggaagagggtagggcagggcagacagtgcaccaggggaagtgcttccacagggcagagctgggtg | |
| cggggacccccagatgcgggcggcaaactggagaccccacaggagccgggactggcttcattcctcattccctgcacctgtgatgg | |
| gcagcagcggccaccggtgcccactgggcagttccaggccgggccgagggactgcacccttttgctgtggggaggggacctttcc | |
| tgtttcatgtctcgtctataaatagtgaaaggatgtggctttgcagaatcgattgggcgtctagcttgccttcctcaataatgcagcgatga | |
| cagctccacactcaggacgccgctgttacccagcctcgtaaaagccctggcagacggccgcccaccctccaggtgtgccagtgcag | |
| ttttcatccccagcgcacaggtgggaaagctgtggctgcgagggcgtggggatctgaggcaggctggcgtcagctttcaccttcatgt | |
| gcaggcatatccagaactttccggatctttccccgggctgaggaggaaataccagcagctttaacgagcggctggctttggggcatca | |
| cacctgtgggccgggctgcgtttgacggcctcgagtttcccaggcagcgctggcgctttctcctgtggggcccggagctccgaggg | |
| gctgtgaagcagggctggctctgatttgctgtgctccccacactgtcccctccctgggcctcagtttccccatctgtatgtgattccccttc | |
| caactacagccagcgggaccctggggcgggctgaaggcgtggtggtggctgtgggccctgcggcgaggcctgcgctgggctcg | |
| gtggtcgccccagctggggaaggggcagtggctgcaggggtgggagggtggagggaagcccttttccaaagttgcctggttgggtt | |
| ctccttgtggccctgccccaccccaccctgctccctgggagcaaagggagctaaggactggtttggggatggatatgtatatggagg | |
| gattctgggtgatgacttattcatattacaaaatttgcctttttttgtttgttttttgagatggagtcttgctttgtcacccagactggatta | |
| gagtgacgcagtcttggctcactgtaacctctgcctcccagattcacgcgatgctcctgagtagctgggactacaggcacataccaccactcc | |
| cagctaatttttgtattcttagtaggggcagggtttcaccatattggtcaggctggtctcaaactcctaacctcaggtgatccacctgcctt | |
| ggcctctcaaagtgctgggattacaggcgtgagccactgtgcatggccacttttttgttttttgagatggggtctcaccctgtcacacagg | |
| ctggagtgaagtggtgggatcacggctgactgcagcctcaaactcctgggctcaagcgatcctcctgcctccgcctccagagtagct | |
| gggaccacaggtgtgcaccaccatgcctagctaattttaaatttttttgcagaagcggggtcttgctatgttgtccaggctgaaaatgtga | |
| cttttgaactaaagcatgctgttgcttgacatctagtccattcacaagttcattcaaccactatctagttccgggactttctcactgcacaaa | |
| acagaaactgtacccaaatactgtcactctgccctgactgccccagcccctggcaaccactaacccgctttctgcctctgaattcgcctc | |
| ttctggacgttgcctatgaatggaattgtgccataggtggtcttttgcccctggcttctttcactgagcgtgatgtgtttaaggttcatccatg | |
| tcacgcgtggactagaactgcattcttctgaggactacattttagcctccagaagcatcagggactggccagaggtggaccccagcat | |
| cccagctcccagacagtccagaggaggcaggtgttgggagggcgtccggggtggcttcctggaagagatggcgctcactctgggc | |
| cggtgcagccacccgacagccacagagccccctagagggagtcctggcatattcccaggtgaacctgtaggatggactggcgtgc | |
| ccgcggcccctgtcctgctcaggactccgagacaggctcacaacctgtccctgggcctcactcatgccagccgccaagctgttcagc | |
| atcccttagcccccaggaggcacacagtggggccgcggtcactgagcgtccacacgcctggtgctgtaagaatgcaaagtagagg | |
| gtctttattgagcatctactacatgctgaaccctggggtatgggtgggcacggcagatgcaggcccccctgggcaggggaggagcat | |
| gccccttaagaaccaggccacaggatggacgggcttggccacaagctaggggtagtcactgcagccttcatggcctcctgtgtgcaa | |
| agtcagtgctgtgggaccccatctgggtgcacctgctgtggctttcaagtgaggcccaccacggagcagcttccacgctggctggga | |
| cagggtcagcggcagggagctccagaccaaaggagcggcggtggctggggcagcggggggcagtgaggctttggacacatcca | |
| tggggcgggcaggcatctcagcccccacccgcccagtctgcctgctccggggccttcccttccctggacagagctctgtgtgattgg | |
| ctccaggctgcttctccccctggaccctcagggtcagcttagagtgactggctatgccctggcctagatccaggcccgtgtgacagga | |
| gagagctaggaacctggcccctcactctgagacacagatggtgtcctctccaagttgcccttgggacggggaagcgccatgtcccct | |
| gcagggcaccaggccagccagtggcccttgacagcgaggccctgtctgcaggcagctgggggacttctccctgtcgggaggatgc | |
| gtgcgtgttagcatcggggtgcctggttgccccaggggtggtgcagggcggttataggcggtcctagcattttggggttgctttcagtg | |
| tatctccgagacctgtggaattctgggaggagaaactccccagcagtgtcctcagtgcgcccccctaccctgtacccgttgctcctgg | |
| gagggatcctgagtgctcccccccacccaccatgcacccattgccccaggagggatcctgagtgctcccctctccacccaccctgca | |
| ctcattgccccgggagggatcctgagtgctcccccctccacccactcccaccctgcacccattgcccctgggagggatcctgagtgat | |
| cccccctccacccaccctgcacccattgcccctgggagggatcctgagtgctcccctctccacccaccctgcactcattgccccggg | |
| agggatcctgagtgctcccccctccacccactcccaccctgcacccattgcccctgggagggatcctgagtgctccccccgacccac | |
| cctgcacccattgccccaggagggatcctgagtgctcccctctccacccaccctgcacccattgcccctgggagggatcctgagtgct | |
| cccccctccacccaccatgcacccattgccccgggagggatcctgagtgctcccccctccacccactcccaccctgcacccattgcc | |
| cctgggagggatcctgagtgctccccgcctaccctgcacccattgcccctgggagggagacttctgtagaggcctcacctctgaacc | |
| ccccactcacaggctcaggggcccagggctgggggcgtcctgccgtctacactccagattgagcccgcacggttaaacagggccc | |
| tctccgcacgccctgcctaatgcaaagcggtggcttcgggtgggtccccaaggggccttgtgaaccctgggggcctggcccctccc | |
| acctggctgcacccgctgggcgggagatcccatcctccgagtcctcagcccctcagcctcagctgcagcccagacaccagggctct | |
| gtggggcgggggtggggcagcggttccctcaggtctgtggggctgccctgctgtggttcctgcaatttcctggaaggaggtctgggg | |
| cctggtgtccagtggggcctgtggggacggactcgggggctctgtggggagggactgggggctctgtggggacggacttggggg | |
| ctctgtggggagggactggggggctctgtggggagggactgggggctctgtggggagggactcgggggctctgtgaggagggac | |
| tggggggctctgtggggagggactgggggctctgtggggagggactgggggctctgtgagagggaactggggggctctgatgtg | |
| ctgggcgtgcacaggggaggctttgcctctgacatccacacctgcagttcccaggcacacacagcggccccatcggggtcctcgca | |
| atcacagtctgtgagtggccactaccaagggcgtctccggatcaggccaggcccagatgctgcccggacccaacccccacagcgc | |
| ctgccgccacagtagggaccaatggccacagaacactcacccggcctcacttgcacggggctgttcagtctggtgggcaccaggtg | |
| ggcgctgttctcgcccagtgggaggcctgccagctccttggttgctagttgagatttttccccaagaaatagtcgtgttgttccttctgctc | |
| cgcctggcactgaggttggtgacacgccccgaccttgctctaattggcagatgagaatttgtcatcagatgtcgaccctgtctacgcag | |
| tcccctggcctcggagtccattgtgcatctctgaagggcttgaattggttgtttaaagctgggtaaatgcccccttgacattctgttgacac | |
| tgtcaatttgctgaacaaactcttccacaggtagcacaggaggccagtctcgccccagcctgagccaaggcccagggaggcaggtc | |
| ctgtgtggagtcagcctttttgtgagtgcagcggccctgcaggatgcaccgggtttgagaagtggactcccccgtctcccgccacatc | |
| ccaaaagccctcagccatgagggcaggacgaagccgacggtcgccctcgtgccgagatgacaaccgggcacagaggcggtggc | |
| tcccagcttcctgccctcttccccgtagtggggttacccagcgtgactcatggcccgagccacaggcacccgcccagggagccacg | |
| gcggggagacctggctttattagagatgcgttcgtgcctcatcaagtccaaaggaggaaactggcgcgtccctcactttccctgaaac | |
| aggctcccccacagcccaagcggtcaggttaaggtgcatttgcagacagatgccctagtaggaggctggagcttttggcccagaattt | |
| tccctgctgtaggcccatggtcaccgagcctgcccgggggctgaggccctgaggaggtgccattgccccacccaagcctcagaca | |
| agtgtcctgccctcgaccctgcgggaggcagcagctcaggccctgaccctggccagagagggcagctctctgaggctgctgtggc | |
| cgtggcaggcaggggctgggcttgccgggccgtcgagtgggcacagagactatctgggaaggaggtgggcggtgagagctgact | |
| ccccgcagccgggggacagatgagccccgtgccgcacagcagggccgcgtggtggggccccccaagttccgctcggtttcagct | |
| gttgacgagcggaaacacacagttactggaaatgagaggtttgggggagccgccaggggttgaacatgtgtggtttccctgacggct | |
| tttcagtaagagatgcttgagctcaggctgggcactggctggtgcgcggagggggctgggccagagtcccccccttgggcctccact | |
| ctccatccttatagagggacctgggttggtcccagctcccggggtgcagcccctgggtggcccctcctacttccccttcagtggtcaga | |
| aggaggctggcctcattcctgtctgacggagagccaggtggggaggccgggcggggctgagtgtccagggtccacatggtggcg | |
| gggctcaggccgtggcgaggtcctgtcctgggcctccagggtctggcccagcacagagctctgcagctctctggtggacgggcctg | |
| gctgggctgtcggcagccctggggccaggtgggcagtgggaatggaggtggaggcaggtgggtggctgcaggtgactggaagg | |
| cttggctccagcctgcaggggctgcagtccaggccggccacccccacgagaccttcccccttccaggtgtggggtggcactgggtc | |
| aacaacacaggccacggacttctttcccccacgtgggccgtgcgaccccgggccgccaccacctctctgagctcccctcctccttag | |
| gagatgggaagcggctggggccatgaagtgtcccccagggctcctgacagccccagctgccgtcctcgtggctgggcgactgccg | |
| cgaattgttgcgccttcttttgagttggctacggagtcatggcggctgtgggaggtgtagctgggtctcgggcggcacgcccggctcc | |
| cccggggatgtgtcatctgtgtcatccaccctggagccccttcctggtgctgtgggggcccctccaggaaggtctctcccagaggcca | |
| gccacaggctctggggagactgtgtgggatttgcatgcagttgtgtgctatttgatcgaggtcatgtgtgggatttgggcgaggtcctgc | |
| gggattgggtgaggtagtgcgagatctttgggcgaggccgcgaggcactgggcgaggctgggcattattcagtgccttggaggggc | |
| agaagttttaggacctgccaagtccagcccaagaatccccaactttccccacagggaaaggtaccccagacagtccccagcctgtgc | |
| cagctgtcttgggacaccctggtgtcccccaggttgtggcactgcaggcccctcccccaggtgcccctccttatgggcccctcccagg | |
| gctgcaggcacctgcctggatcccacttcccaagtgtggggtaatcccaggcgcccggtgatcacgctgggcccgggcagagtga | |
| ggctctgagggggtgccaggggcacagtgggccagtatgtgagccgggctgggcgctgggactgggccggcaggcgagcggtg | |
| ttccgagaggaggattcctgggctcggaggcccctctgtgcctcgtgtggtcagagagcagggagccggcgggtgtgatggggat | |
| gaggtgacctctggggccaccactgaagccgccgtccatctgcggccgaatcgggaggcacacagaggtgtcctggtggtttcccg | |
| ggacaggcgggaggaggcgcaggagggcggcgtctgcccccgggatgccaggagtgctcctccggcagcgtgggcttcggctt | |
| cgtccctctttcaccagtgccgacgccccgcgggtgctgtgggtgaaggggcatcggtgccctcctgctgggcgggaggaaggaa | |
| ggggagcgggaggctgggtctctctcgcccagggctctgcccccaccaccctagggttttgtgtttcggtggagctgtaagaatgcttt | |
| gtctgctccagacttcccggtcctgggagatcaggtggtcaggcaaaacatccagaccgtctgggtggggctgggggtgcggggg | |
| acacgcgccttcctcctctttcccagggagcttctgccactcctgtgccaccctggggagctgccttgccctgccccgctctggtcccc | |
| atcagcaggtgtggttctgagctgccttcagatccgggtggtgtgctgggcatagctgccccgttttctcacctgtgaaatgggcccatt | |
| acacgcccccagggcggttgtgggggtcccatgagacagagcttggaaagcccgagtgtaggtcaggccaggagggttgtgggct | |
| cagtttcttcactgctgggctggtgtgtgccggacagatgtctcgggatcccttccctccctgcagcattcttaacaaggacgctggaca | |
| tggagtcagcacctccgggcgactgccctgctggctgtggggctcactccagaggagaacaggagcccctccggggagccccttc | |
| ctgccacccccagcgacaggatctgttcaggagcagtggtgggaggttgccagaagcatgtctggcgctggcccggagcgcagcc | |
| tgtgatgcccgacttcattcatggagtgtggtttgagccctccagcagcgctgctgtgggcctgtccattgccttccctgcctctgagcc | |
| atcgtggggtgcagggctgggctgggtcttcttgggagcagagccccggcgtgtcatgaccatcttggcctctccaactcaagaggtt | |
| tgtccacatgggtccctaggggccgttggggtgacccagggcaggagacacatgcctgcttttgggggatccgcgtgggaaattccc | |
| gcagtggagcagcaggtggggctccagcaggcgtttttcacactccaaacagcctgcgggggcactttggtgacacctagttcctgg | |
| ggtctcagagcctgctgggctgtgtcttggcattttggggaggggctggccacatgcccccagagtggccaccccagctccgccatc | |
| tccacgggccttctgtcggtcagatgcagacagccgttcccatgtcgggtgagggcattgttttcccggctgcgggaaaggtagcttc | |
| ccgggaggacaggttctgtccccatcccgcctcccacacaaagggtttccccggagccagaggaggaaacctggccttcctgcactt | |
| cctccttcgcacattttaaaccatccgaggatgcaactgggggaaaagatttgcagacaaaagagctgggggtgccggcaacagctg | |
| tttgggccggaaagcctggccccggctcagaccccggctgggtccctgcgctgtggcgggccggccgcgacccccgccccaccc | |
| aactccgccccctgctgcccgctgtctccggagcggcggctgtttggggctggctccttttctgggcccccacctccctgaagccccc | |
| actgcccctttcctccttcctggggccctgggagccagggcagggcccccgtgcagctttcagcagctccagcccaacggctgcag | |
| gcgctggggaagaccacagctcaggtgggaccgcaggtggtggctggaggcagccatcccacatcgcagcccccaatctcgtggt | |
| tttgtgctccaacggtgaacaggcctggtggtgctgggctgtcgtgggccccttggccccctcggtgcctgcatccctctctcctgtcc | |
| aagatggggaaggaagccgggagcagaaagggaagaaagacccagcggctgcagggcgcctttcagggcctcctagccaccgc | |
| agaggctccagccagctgtttgaacaggggttgggacacagggtcctggcaggtttgaggagcccggggccttccttgctatagctc | |
| tttgttctgggggatttggggaggccagggaaggggctgtgagcaacgaggttccagcatcttcccaagcctcctccatcccccagg | |
| gctgggctgcttggccgttccagtgggagaggacccgaggtgggaccccgagccccctgcccagctcatttcttagcagccggtgc | |
| ctcctggaaggtgggtggtgcccactgggattggggaaacttggtctagcctctacccaagggaggggctgaggtcccaggactcc | |
| ccctgccccagaagccttggtccgggctagagctgggctcgttggcctcatggaagcctgccttgggaagcatccccacccaggtct | |
| ggccttgtctccccgtctccagtagggggtcacagtgtccccccagccagtccacatatggccccatttgcttgggcaggccgatggg | |
| tgggcctgtctgtgtggcccaggtcacctggtggggcccactgtcagtggcgtaggtagaaggtggtcagccttgctgggtcttagca | |
| gagcacggcaggatctgggtctggggagggcgtgggggtgcccctgccatcacaggagcaggcagagtgggagccctagactc | |
| ccctctgggcagaaagccccttgaggctgggggcaggtcctgctggtgagggaggggtcccaggggcaggggcaccctcaggct | |
| ggccgcctagcgattggctcctgcaggacgtggccacgggctccctgggagcagcagctgttgggggtgtctggggagaagaccc | |
| ccatttattgccctgtgagggaccacactgctgccaggggacccttgggcttctgtggcaggacagtgggccgggattggcagtgag | |
| gcccaaggagggcaggtggggctggacctgtggctctgctggggagcaggtgggatgtgttaagatgctgatttcagccgggcag | |
| ctcctccccttcccttcccagggagccggggtgtgcagggccgttccttcccctctgggaagtgcagctcctgtgcaccggagaggc | |
| ccccgcactgtccctgcccgtcccaccaactgtccctgcccgtccctccactgtccctgcccatccccctcactgtccctgcccattccc | |
| cgcactgtccctgctcatccccaacactgtccctgcccatccccccctcaccgtccctgcccatcccccccgcactgtccctgcccatc | |
| cccccccactgtccctgcccatccttgccttgggaggtgctgtcacctggtggagccatgggaagcacctctcccacccagggtctct | |
| ggttcctgtggcgggtgcagagccagccacatcactgacaccccagctgggttgggtccagctgaccccacacccccacccctctg | |
| ctcggcctctgcctgtgcccccatccctggccctgcatgaccataaactccttgagggcaggaagtgagtgagtcctacccgcactga | |
| gtccctctcacccctccttctcattttggggcggagggaactgtcttctgtcccctgggggtggcagatggccgcttagcttagaaagg | |
| aaaacagcacaatttagaattcgtcctgggtgaaaggtctcggcagcatctgcctcctgctgctttctggagtcgcatttccatggagcg | |
| gccctggcagggagggtgggagtggcacagtggaaatactgaggcgggaacaaagtggtggcggtagaggtggcgggagcgg | |
| cacagcaggcgccggggccacttggcaccctccccccgcagccctcctggagtccccgcccgccgctcacctgcggggggccca | |
| gataggacaggttaccccagcgccgccaggccactgagcgtgggggatgggacaggcaggtgccgggtgaccctaagagggac | |
| gcacggaaggggagtccacgggggcacacctggcgtgggttgggtctcctccgtgggagcccgagagctgggtggcttggtgga | |
| cagggcagctctctcgtccagtcgggggagactggcgttccctatctcaagcctcactttttagccccagaggggtctccccaccgcc | |
| cctgataggaggagatgcggggacggtggaaggtggggtgcgggtcactcatggaagccctgaggatccccgtgggcgcggca | |
| gctgttgggtgccacctcactcggccaagccctggcgcccacctcgcggcccacacccacctggccgccgggacagcattgaaga | |
| cagagtggcccgggtaccgggggtgtggccagcgccaaagctgtgaaggatttgctcacaggctggtggtcctggccatggccatg | |
| ctgcaggcctgaggtccctcccaccttctcagggacgtctgggtgctgtgcggagggcaggtgttcgccgtctgtagggtgctccag | |
| ctctgtggccatgtgggagagaggtacagactggggcgggcacaggctggaggctgcccacagcagcagcgggagactgggcc | |
| caaagctgccccccacacacagacccccacacctggctcaaaactttctccaggctcggcccggttccccccagctagcgcgtctca | |
| cggaacctccaagccgtggctgtggggccgtggcaggggccttccctgtggtgtccgatgcccaggctgggagtgggggcggac | |
| aatgggccctctcagcctgcattcccccacgggagcctcctcaggcctccggctggggctcctgccccacccgctgggagcagctg | |
| ccgccacgtctgacacctgctcccagccgccctttgtccgagcaccgacgcctgatttatggggcgtgttttcctcttgctggcccggc | |
| gtgatacagcctcgttaactgggatgcgtccggttgggcagattctcccacagtcctccacgttggggtccgcgctcagggtgggga | |
| gtggggcccggtgtccgggcagagtgtcgtggtccattcagggacagccaatgtcctgtgacctcagggcacttggtgggagactg | |
| ctctaggggctcggacctccagcgtctcccctctggggtcccctaaagctgtgggatgggcctcccctgcccctcctccccgtcttgc | |
| ccagcaggtgagcttttcccgctccttggagcctcgaggggccgggaggcaggggaccagcttgcatgggccaggctgaggagc | |
| ctccagccccggctttgatgaccgagcgccctctgtcctgtacccaccccctgccgacttcagcggctgggaaagcaaaaagggcct | |
| tttctccaagcgggagctgtggccaactcccgcctgacaatggggcatttatgtggagggagggcgcccctcctgtggccacagccc | |
| agcccagcccagcctgtgcactccacgggttcccagcggccgctccacctcggggaccggccagcctgtaactggatctgcatgca | |
| gtgggaaccacgtctgccctcccaacaggtgcccgcagaggcaggtctgtcgctgcagccccggagtgaccggccacatgggatg | |
| gaggagggggagcctcattccggggctgtgcccgtcgctcactgggctggcaggagccgtagcaaggcctgagtgaattccccag | |
| agcccctttggaaggggtgacatgtgttttctttaagccagttggtttgggacttttggcttctggcaacagatggtcggccagagtggcc | |
| cttttgcgttctctctacccaagccgtggtcacgggtaaaagccttcggggaagacagatgcctcccgggcctttgggacaggggcgt | |
| ggctggctgctgagaaacgcacaggaacccccacccccagcctccacaagcccctcggggtaaggcccccccaggccactgcca | |
| cacatagcacaggtttgattggagacactggggtgggaataagcgggagaggaggcgaggacttggggggccggtgggggtcct | |
| gtgggaagccggcagcttcagggccggtcctggggcaggcggccgccgcagccccgcacccaggtgggctccggaaggcggg | |
| acggttccctctgtacatcccatctggaggaggccatgcccacccagggcctgagccgtgaggctgctgccttcccgggccgctgg | |
| ggccgctgaggctggtggcatgttctgcttcagaggcttcaccactgggaagagcggcgcccggcgggtcccagaccttttcccgg | |
| agcgcaaggccaggaggtgactggggccggagatttatgaccacagcaggtggctggggggggcaggctctgcgcctgttgccg | |
| cagggaacaggtgagaccaggcctgggtggagatgcaggacccctcccctggctccccttgttctctctgagcccagactgtgtgtc | |
| cggggtgaggggcagcctttcacccaccttgacggggttgcagagacctggcagagggacctcctcctggcaccttggtttccccg | |
| gctgtaatgtgggtgcttctgctcctggctgatgtgcctggatgtgagttatgaagactcaatgccgaaagggaaacgtgtctgggggc | |
| tgagcgtgggcagggaacgggccagccttcatccccagtgcgatcttgacatgagatgtcccatccccattgcagtctcagcctgag | |
| gtgtcccatcagaaaggcttcctggccgggcgcggtggcttacgcctgtaatcccagcactttgggaggccgaggcgggcagatca | |
| cgaggtcaagagatcaagaccagcctggccaacatggtgaaaccctgtctctactaaaaatacaaaaattagctgggcatggtgtcag | |
| gtgcctgtagtcccagctactcgggaggctgaggcaggagaatcgcttgaacctgggaggcagaggttgcagtgagcctagatcgc | |
| gcccctgcactccagcctgggcaacagagcaagactcaaaaaaaaaaaaaaaaaaaaaagaaaagaaaaagaaaggcttccacgt | |
| aggaaggggctgatccacactttccgtggctgccatcatgttgacacctgaaggttggaaaaaccacggcctttgttactgcccaagc | |
| aatgcctgttcgctttagcaaagctgggaagaaaagcagataggcctgcagacccggaggctctacggagctaatgcacccggctc | |
| ctctccatggctctccgcgggtgtagacccagacccctgcccgggctgtggagtccaagctgggttctgctggaacatcctagtctcta | |
| gcttgttccccaagttagcagtcatttgtcttcttctttctttctttttttgagtcttgctctgtcacccaggctggagtgcagtggcgtg | |
| atctcggctcactgcaacctctgcctcctgggttcaagtgattctcctgcttcagcctcattcagtggcattcagtacattcccaaagttgt | |
| gcaatcgcggcatgtgtctggttccagaacattttatcagccctaagggaaccccgctaagcagccgttgtgcccgtgcccggggtgtgggg | |
| ggaggcggctgtgcccatgacaggttcccggtgctgagtggcgggggcgctggggaagcaggtgggaactaactgccctggcac | |
| atctgccaacagctgtggcggggccttcgtgggcccgcgatgccaggaccccaacccgtgcctcagcaccccctgcaagaacgcc | |
| gggacatgccacgtggtggaccgcagaggcgtggcagactatgcctgcagctgtgccctgggcttctctgggcccctctgcctgac | |
| acccctggacaatgcctgcctcaccaacccctgccgcaacgggggcacctgcgacctgctcacgctgacggagtacaagtgccgct | |
| gcccgcccggctggtcaggtgagggaggacccacagcctgaggggtgggcagacccaggcccgccgccagctgcccaggcaa | |
| cttgaggtacttggcgggatccaggcccgggtctctgacccggaagtaattgggagcccctgccccagggtccctgcccccctccca | |
| gacctggaatcgcctcgttttcagctgcttgctctggcccaactcttgctgtccccgcttctgcgagacaaggggagcctctgtgtccag | |
| gccgcccctgcccccttcctcctgccgtgcactggccctcacctgttagggtgaagggtgtctggaggtgtcagctctgggaagaga | |
| cggcccaggctcaggcctcttgggagctgtggtccttcatctgccaaaggcgaccggtctcacggcaggtcctgaggatcaaatgtg | |
| atagcacctaaggtggagcccaggctggcacctccaggtggcctgaagggagggaaggcccggcctcggggagcagtgaggcc | |
| agcacgaccctcttgtccccttgtctccagggaaatcgtgccagcaggctgacccgtgcgcctccaacccctgcgccaacggtggcc | |
| agtgcctgcccttcgaggcctcctacatctgccactgcccacccagcttccatggccccacctgccggcaggatgtcaacgagtgtg | |
| gccagaagcccgggctttgccgccacggaggcacctgccacaacgaggtcggctcctaccgctgcgtctgccgcgccacccacac | |
| tggccccaactgcgagcggccctacgtgccctgcagcccctcgccctgccagaacgggggcacctgccgccccacgggcgacgt | |
| cacccacgagtgtgcctgcctgccaggtagtgctgcccgctggccggggtgcacgagcccctccccggctgccaggcccagggg | |
| gtgggaacggggcgcctggtgtaggggcagccctgggagggcccatggcggggagttgggaaggcgggatggcgagttcccca | |
| gactgcgtgtggtgtcggggggacgcgtctggagcggggtgtgcgctgggaggaggcttgcacagggtccaggtgccagcccgt | |
| gtctctggagtttcccggaactcacattcgagctgtaggacgccctgcctccccccagtgcaggcaccctgacttgcgcaacccccag | |
| ggtttcccctgcagtggggctggggccggagagggcgtgggagctgcgggggcaccatcacttcccaagccatggcccttcctgc | |
| catcctgctggtctcctgcgggtgtgggtgacaggtaggcgcagccgcctgcccgtgtccccagtgctccccgatccccgtgttgtac | |
| gtaggagcctctctgctgtagttcttgtttgagtaaattttgcagcagctctaaaaataaggaagagttttgaaaacctccagtctcggccg | |
| cgcgtggtggctcacgcctgtactcccagcactttgggaggccgaggtgggcagatcacaaggtcaggagatggagaccatcctgg | |
| ctaacacggcgaaaccccgtctctactaaaaatacaaaaaattagccaggcatggtggcacgcgcctgtagtcccagctactcagga | |
| ggctgagggaggagaatcacttgaacccgggaggcggaggttgcagtgagccgagatcccgccactgcactccagcctgggcga | |
| cagagcgagactccgtctcaaaaaaaaaaaaaaaaaagtgaagagaaaaaagaaaacctccaatctcagctgcacagaatggctgt | |
| taggtgggaatggggagagggcattcaggcaggggtggggctggagggtgcacagggtcctggctgacccacgggtggcccaa | |
| gggctgccccacccccttggaagccccaagctggcctggcccctgcaaaggggaagctccctggctgcccagtcagctgagccca | |
| gccacctctgcgtgcagacaccagacgtgtgcgtttcctgcaagtcactgggcgagcgtggggtggggacagacactccctggcca | |
| gcttggatctctgcagggtggggcgcagagcccttctgctggtgggggctcagatgccttcctagcagcacgggaccctacctccag | |
| tcacacggattgaggggccaagctcttggggggccgtggttcaggctagggcaggtggggtgtgtgcggccttgtccaaggccacc | |
| tgtgtgcagagctgatgcccacccgccttgggcagggtgggtgaggacctcctggtgaggcccagcggcagagatgccaggggtg | |
| ctgggccggctcagagccactgcccgtgcaggggcagctcagggccctggggagtgggcagggagggaaggggaggggctgg | |
| tggggttcactcacttgagtgtgggtagatctcagaggctggggtgctgggggctgggtcacccgtccggtgaggggtgtgctcact | |
| gcctgctgtggagtctggccgtttcagggtgagatgaccatccgggggcctccccaaatgactcaggcctgccagaccccagcttca | |
| gactgtccccggcggcccctgcacccaggccccctgaagggccctcagccacctctgggtggtcaggagcccaggagacagctgt | |
| gtgtggagtcacggggaaccgagatgggggcggccctgagctctgcaggcccgggaactggctgggatttggggtcgcacagcc | |
| tgcctggcacgtcagcactgtctcctcgtggggtcgctggccgaggctacgtcgcagagggtccagctggggtccacggggctctc | |
| gctggcttcccgccctctcccccactgtgtgcctcagccagtgctttgcagggcagccctggggtcgcaggcccagccaggggccc | |
| catctgcctcggagccggggtgcagggccccacacatgagtgggaccgggactcaggctccaggctctgaattttttttttttttttgag | |
| acaggtctctgtcactcaggctggagtacagtgacgtgccctcagctcactgcagccttgacctcccaggctaaagcgatccttctgcc | |
| tcagcccctcaagtagcttgggaccacaggccggcgccaccacgctttgctacttcttgtatttttttttgtagagacggggtttcgccat | |
| gttgtccaggctggtcttgaactcctgggcgcaagcgatccacccacttcagcctcccaaagtgccgggattccaggcgtgagccac | |
| cgcgccagggctggggtctggttttgggtgctccccaagctgatttgcattttggattttggttgtctctggctgcctgcttttgctccggtc | |
| cagcagacagacagaacggacggggtggtggcggtggggcaatgtctcagagggaggggccgagcaggtgggagctccaagg | |
| gcaacgggggttcaaaggcatctcgggagggcggcaattagtaaccggctgcaggtttggctgttgccgaacgcgcctttctgggtt | |
| gatcattgaaccagcgggagttgagtggattaatagctaactggctgccttggaggcctgggtggggccatacctcgggggagggg | |
| gccctggcagcctgtggaggaggagggggaggaggaggtgggcaccagagctctgccccagggaggatgggccatctgaatct | |
| ctgcagcttccctggggctgcgaggggcccaccctgccccagccagcagcctgctggccccatgtccccctgctgctcagagccca | |
| ggccccagtgtcagcccctgaagatggggtgtgcggcccccctgtggagacattgtagggagctctgagcatgcaggcggcaccc | |
| aggtgcaggagctgccggaacctcttaaaagagacccaagggtttttgtgacagaatcttctggaaccctgtgttcctgagtccctgttt | |
| agccagtcctgtccaaaatgattctttcaagcagggcgtgtccctctgggcactggagtgaggcaggggacgggcacagaccccgg | |
| gggccccagggcagggccgtggaggccaggctcttgtgtccagagcagtgtgtcgggggtccaggcaggagccggactggacct | |
| cagggaagaggctgacccggcccctcttgcggcaggcttcaccggccagaactgtgaggaaaatatcgacgattgtccaggaaac | |
| aactgcaagaacgggggtgcctgtgtggacggcgtgaacacctacaactgccgctgcccgccagagtggacaggcgcgtatacgg | |
| gtcgccgcagggcggggtagccggggcggggctgctacccacttactaaactgcactcacaggcatagcggggagcagccgaga | |
| ccctgtcccaggccaggcagactagggttggccgagaaggacaacatgatgggggccccacttcccgtggccagggtctggtgcc | |
| cctcacctcgggggtcagcacagacagggccctggttggtctgtgtgggccgtttgcaacctggctccaggcagcccctgtgcccag | |
| tggggtgtcctggcattgagggccttcagcaccccactcaggccacgcttcctgggccagtcacagggatgcctggatgaacagac | |
| aggccctgtgttcgggaattaccctctggtggggtgacgggcaaaggtgcaccccgacgcagcagtgtccacagagcacaaagag | |
| gaggcaagactccatggtgctgggcctgcagaggaaggagtgatttaggctggcggtgggtggaggagtcagcccaggaaggctt | |
| cctggagggggcggcattgccaccctgcgtcttaggggacagggagctcagggagtggcagctgcccggggccgacagctcctg | |
| ttccctgcaggtcagtactgtaccgaggatgtggacgagtgccagctgatgccaaatgcctgccagaacggcgggacctgccacaa | |
| cacccacggtggctacaactgcgtgtgtgtcaacggctggactggtgaggactgcagcgagaacattgatgactgtgccagcgccg | |
| cctgcttccacggcgccacctgccatgaccgtgtggcctccttctactgcgagtgtccccatggccgcacaggtgagtgccttgaggc | |
| ccaggtgggcgcagggggctcacatgggccaggcctgagctgtgtgacctcacccagggactgtggccctcctgcacccaggata | |
| acctgaaagggcctttctggtcagggaggccggtggtcgctgtgagtcccccggaacgtcccaagtgtcacgggatgcccgacgg | |
| ggtcacggcgggcagctcctcctggggtgtcagtggggttggaccccagccgtgggtggtgtgccatgcctggcccaggggccgt | |
| gcccactgaccgccgctgcctgcccaggtctgctgtgccacctcaacgacgcatgcatcagcaacccctgtaacgagggctccaact | |
| gcgacaccaaccctgtcaatggcaaggccatctgcacctgcccctcggggtacacgggcccggcctgcagccaggacgtggatga | |
| gtgctcgctgggtaggtgccagcacagggggtgcggccaggtgggggtggcagcctggccccagaaccgatgagaagtcgattct | |
| ggcttcagggggtgcccagttagatgggggatgggaccctgccagtccgatgggggtggtgtgcagtgaggtgttgcaggggcca | |
| gggtgcctggagcagcctctcacccgtgtgccctcgccaggtgccaacccctgcgagcatgcgggcaagtgcatcaacacgctgg | |
| gctccttcgagtgccagtgtctgcagggctacacgggcccccgatgcgagatcgacgtcaacgagtgcgtctcgaacccgtgccag | |
| aacgacgccacctgcctggaccagattggggagttccagtgcatctgcatgcccggtgcgtcggccggggccagggcgggaaac | |
| cgagtcgaggctgggcagccttggaggggcagccccgggaacatctgtgggttgcttccgctttccccagcctccatgccttctggg | |
| cccacagcctgcacagggcatggggaaactgaggccaggccacagggccagcagagcacgggggcagtgctaggcagatgag | |
| ggtgccgggccaggggccacgggctgcaggggagcgggtgcgcagggggcccgtggtggctgggacactgggctggaggca | |
| gggttcgtttctgtcccaagtccacagctgtgcccagtggggcactggggccgcgcgtgcccccctcactgttgccccaccccacag | |
| gctacgagggtgtgcactgcgaggtcaacacagacgagtgtgccagcagcccctgcctgcacaatggccgctgcctggacaagat | |
| caatgagttccagtgcgagtgccccacgggtgagggccacccccgccccctgcccccgggtcgtctgcaccctggcctcctgagg | |
| ggtgcctgggcgtgggttgtgtcccctgcccccgggccatctgcaccccggcctcctgaggggtgagggccgcccccaccccctg | |
| ccctcgggccgtccgcaccccggcctcctgaggggtgagggccgtgggttgtgtcccctgctcctgggccgtctgcaccccggcct | |
| cctgaggggggcctggccgtggattgtgtcccctgcccctgggccgtctgcaccccggcctcctgaggggtgcctggccgtaggtt | |
| gtgtcccctgcccctgggccgtctgcaccccggccttctgaggggggcctggccattggttgtgttcagattcccaggagagcgagg | |
| gttttgcctcatgagtggatgggagtgttttcagacttccccgaaggaagggcagggcccagtggggagtgggagcttgcccaggg | |
| gtcgcggtggagcccagggccaggagatcctccttgatctgggtgcagccccttctgcctggagagaagggtataccaggagctgc | |
| agtgcccagacagggaggaggctccagcctggctttctgagggctcgatgctgggagggaggccagcctgccctgtcctgccatgt | |
| cccctgcccaacggcacgccagcgacaagggtatgcagagaaccacgtggggcaggttggcctgcaatggagatgatggctgcc | |
| cggggcggacttggaggaactctcaggggcctgggtgaaaggtgtttccatctgtcccagagctgggggccggggcgcagagccc | |
| agggaggcgttgccagcccaagcctaggctcccaagacatagattacccgtcccagacactggagcgaggggagccccattctca | |
| gcccggctccactgtagccatagcaacccagtcggtttgggaaaaaggccccttctgtgggagcgagggcgtgtggtgctggggga | |
| ggggctttgatccggggagcgggcagcgggaggcagggagagctggagctggctgagctcaggctgagtgtgccctggggagc | |
| ccagccaggcgctcactgctggggtctggccaggggtccctgaagggccatagtgctgttgcacatgactccctcccctcccctccc | |
| cggccccaggcttcactgggcatctgtgccagtacgatgtggacgagtgtgccagcaccccctgcaagaatggtgccaagtgcctg | |
| gacggacccaacacttacacctgtgtgtgcacggaaggtgcgggctggcgcccaccagcggggagggactggggacgggacag | |
| ggcactcagggcagtgaaactgacaaggtcatggacaccttggtctgggccacctggtccagaggccgagagcacagctcagccc | |
| caatcctgagtaggtgggaatgccacgctcggagccctcctcaggttggcacaggtgaccccaggtctggtcatgggtgtcccggg | |
| ggccgccagtcctaagtcttcctgtgcccgcccctcccacggtccagggtacacggggacgcactgcgaggtggacatcgatgagt | |
| gcgaccccgacccctgccactacggctcctgcaaggacggcgtcgccaccttcacctgcctctgccgcccaggctacacgggcca | |
| ccactgcgagaccaacatcaacgagtgctccagccagccctgccgccacgggggcacctgccaggaccgcgacaacgcctacct | |
| ctgcttctgcctgaaggggaccacaggtggccggccaggcgggtggccggcggggggccagtgggcagggcgggcctgagga | |
| ctgaccgacacgtgccaccccttcaggacccaactgcgagatcaacctggatgactgtgccagcagcccctgcgactcgggcacct | |
| gtctggacaagatcgatggctacgagtgtgcctgtgagccgggctacacaggtgagcggccctgcacgtgggggctgactgcactg | |
| tgctcagaggtcaaggtcagccactctgccctggggctgctgaggggttgggtgagggctttggtgctgcccacctgggcagaccgt | |
| ggtctgcagcagagattgcagcgggaccagggctataactggcagcacgatgggtccccccgacccctcttgtctccggtcagagc | |
| tgtcccaggccgtgttggcagagtggcccagcaggccaagagggagcagccagccctgaggccaggcttaagtaccttctgcctct | |
| gtggctcaccagcacctttgagcaggtccctctgcctctctgggcctcagattctcatcagtcagggctgcagtagcccctgcccctgg | |
| ggcccctgggaggatgacttgagtgggcactggtgcccggggaggctggcacatggcacattgtgatccaagatggtttctgacacc | |
| tggaaggatgtggccagaagggtgatcgccccggctcaacagacagggaaatcgaggttgacgtgggtgggaccccctgggcgc | |
| tgggcctcggagtctgacccgccctgcccttagggagcatgtgtaacatcaacatcgatgagtgtgcgggcaacccctgccacaacg | |
| ggggcacctgcgaggacggcatcaatggcttcacctgccgctgccccgagggctaccacgaccccacctgcctgtctgaggtcaat | |
| gagtgcaacagcaacccctgcgtccacggggcctgccgggacagcctcaacgggtatgcggcggggccgatcatggggacacat | |
| cagtcctaaaccctgggagctctgctgccaggagggtggcacctgcacagagcttgagatgggccagaaacgggcctcggacgg | |
| ggctgggtggcagggagctcccctcggggacgcacatgcctctgggtcctcagtcagactacagtccacacccagcagctgtgtgc | |
| tgcgtgtccttgtgggctgcatggtgtgctcaggacacaggcccacagtgaccctggaacacgtttccatggtaacagccgcaagtgt | |
| ttctgatgcccatgatgtgcctggcagcactctgcctggatgggaccatttaaatcagcagggaccccattgccctcatttggcgcaga | |
| gaaggcacgtgctttgttcggggccacacagcgggcaagtggtggggggcttttcgcagcaccctactgccctgcacttggcatggt | |
| cccctcgctaatgaaccaaacccctgccgcagtcttgggtatgggaagcgctgcggcccccacttttttctttattttgagacagagcctt | |
| gctctgttgtccaggctggagtgcaatggcatgatctcagctcactgcaacctccacccctcaggttcaagtgattcccctgcctcagcc | |
| tcccgagtagctgagattacaggcatgtgccaccacacctggctaatttttgtatttttagtatatatggggtgggggagtttcgccatgtt | |
| ggccaggctgatcttgaactcctggcctcaagtgactcagccacccccgcctcccaaagtgctgggattacaggtgtgagccacagc | |
| gcccagcctgcccccactaaaggactctgcgagtctgagtggatgggcacctccgccagcccatagggcattgcagacccgggag | |
| tgcccaggccccggccgtgctgctcgggcctccctcgacctgcagtgtggtcccccttgcaggtacaagtgcgactgtgaccctggg | |
| tggagtgggaccaactgtgacatcaacaacaatgagtgtgaatccaacccttgtgtcaacggcggcacctgcaaagacatgaccagt | |
| ggctacgtgtgcacctgccgggagggcttcagcggtgagtgggctgcgcgtttctcagtgcagagggccgccctcgagtctgggga | |
| gctggagacacaggatcgggacccaggtccagccagcaccatgcatggtgtctccctcccgtggcaaagccttagctccaggcctc | |
| tggctcttggagatgaggaggggcctggggtggggagcaggcccagggctgctgttgtggggccctgccaaggtgcctgggagtg | |
| ctggacatgccgagtgctgtccccttccctccaggtcccaactgccagaccaacatcaacgagtgtgcgtccaacccatgtctgaacc | |
| agggcacgtgtattgacgacgttgccgggtacaagtgcaactgcctgctgccctacacaggtgaggggtgggtggggcctgtgctg | |
| gagggggcggggcctatgtgggaggggcgggacctgtgctggagagggcggagcctgtgctggagagggtggggccaggggt | |
| gggggcggcctgggaagccgtgactggagggtaaagtggtggctttcttggggcggggccttccagaaggttctgcgcctcactga | |
| gtgccttaggcccctgcagtatgtgggccttctcccgggggtggggttggcagattagcaaagatacaggatgcctgggagaaaattt | |
| tcatgcaagcatgtgttttacctggcaagcctgccgcaagacacaggtaagatgcaggtgaatccttagcagggccgggcctgggtc | |
| actgggctccgtgctcccggcgcccaccctgctggggtctctgggggccgggcttccccacccccgccagccggacctgagggat | |
| tcctttgatggcgggctctctttgtgtgagcggaacaaggagccctctgtttgggctgagcggggagggggatgttcccccaactgtc | |
| cagcccctgctatgaacccagccaaggggatcacaggaaatgatctcctctagaaaagacaaagaagagtccacaagcaccgggg | |
| tcctggcgctccccaggagcgggcagagggtgctgccacggggcctggtcagggagggcctgcctgggccaggcagccgggct | |
| ggaggtcccacgcctgcggttcccatgacatccagcagcctgtggccagaacgctgttttctttgaccgaagttaaggacctgggtttt | |
| ctctcctgtgtcagtcagtgaggaccctccctccacggctgggcgcccctgtccctttcagacacgtcgatcgggccggcaggagga | |
| gcggcggtgctgacccgcttcctatctgcccgcttcctgcttcctttcgcacaattgtcggaaactctggcgcagttcctgcctgcccgg | |
| gtcaccgtggaaaccaataggaagagaggccctggaaagtcgccccagcaggccagggagataatgggggacagggaagataa | |
| cgcagtccccacggctataaacaggaagctcggccatgggcttgtgcagaaaaaggccctcggctgtgcacgtggttcccacatag | |
| cctttgccagacaccctgcgtggccctggaggtctccaggccgtgccccgtgccccgacacgcaggatgtgctgggggcctgggc | |
| ctgtcccctccatcgcagcccttgtttgttggcctcccgtgatgggcgtttgctgcctgtgacacggagccccaggattagtttcaggcc | |
| tcggtctcaggccagggcctttcctcgtcacccagagatgagggggcttgcgtccggggtgggcccaggtccctgcagtgtcgtgg | |
| agcccacccgactgaggccgcctctcctggtgcagtccttgttcctgggcacgcctggtggggtgaggcctaggctgtgccccgctg | |
| ccaaccgggaatgaggggtggcttctgagcgtgacatttgtgcagcttgtcttcagcgtccccccatctgtgctccactcctccctgggt | |
| cagggtcctggcacagccgggagcgctcaggggtctcggtgcacatttgcctcccggcgggaccagcagacggcactctgatggc | |
| ggaaagaccagcaggcggtggccgatttgggagatccctctgggtgaggctcccggggtcacgtgtgtctccttcccgcaggtgcc | |
| acgtgtgaggtggtgctggccccgtgtgcccccagcccctgcagaaacggcggggagtgcaggcaatccgaggactatgagagct | |
| tctcctgtgtctgccccacgggctggcaaggtgaggctggccagggcccggtgagggctgggatgggaggtcaggatgtctgcgg | |
| gacacaggcagctcccaggcaggctagatgagtctttgaagaggagctggtgggtgctgaggaggccctggtcggagaagttctg | |
| gaatcaggaattgacctgggagcaccgttcccaactccagttcctgtgaccttcttaggccaaaattaggggagaggggatggtcctg | |
| gggtcacggaagcctactcctgggtcgggagaggcactgtaggtgggtgggccagcctgggaagggcctggagggccaggggc | |
| cgctggtgaccaaccggcctcctcctgccccgcagggcagacctgtgaggtcgacatcaacgagtgcgttctgagcccgtgccggc | |
| acggcgcatcctgccagaacacccacggcggctaccgctgccactgccaggccggctacagtgggcgcaactgcgagaccgaca | |
| tcgacgactgccggcccagtgagtagccccgcggctctggcctcctccaggaagctctcaggcctcagttcccccgggcagggttg | |
| gtgcgcatggtgctggccataagcacccagggaggccggagtgtggccggggagggttgggtcaggtgtgggggatgtgctgag | |
| ggatggccagggtggttcaggagggccccagagccggcttgctcctccctgcacttcgcggaagagtcacgagagcccctcccac | |
| ggctcttcactgagccgaggatggctcgggcccggcctgcactcccgcctggctcatcgggccccgctgcaggccagggctctca | |
| ggcctcccagccctgcttcacagaggttgaggttggcagaagccgggggtcttgctggcatcacctggcaggcagaggcaagaact | |
| gtctctcctcccctgctcggtctgtgaagcctgcaaagctgccccctgccctggagcttgaggacaggagcaggtggggagagaga | |
| ccccaagcacaggagacgggtgtgacgcagcctgtgggtgctggggctccccaccagacacctttgtcacagggctgcttgccgca | |
| gcctcggcaacgaaaggctgggctgtccccagccatgcagccttcccggccccctcccgcaggtgtgggtttgtgcctgcccctcac | |
| actcacccttccgtcctctcccagacccgtgtcacaacgggggctcctgcacagacggcatcaacacggccttctgcgactgcctgc | |
| ccggcttccggggcactttctgtgaggaggacatcaacgagtgtgccagtgacccctgccgcaacggggccaactgcacggactgc | |
| gtggacagctacacgtgcacctgccccgcaggcttcagcgggatccactgtgagaacaacacgcctgactgcacagagaggtgtg | |
| cggggctcgagtgaggccgtggaagggaacgggcggtgcgggccacacgcagtagctggcagcctggcacaccatgcaggcgt | |
| ccgcctaggccggctgggcacttagcgctggctgcattgagtccagacattgttcattgtaccgacctgtccctcctgagagggccatt | |
| gtgacctcctcctgtgtcccgcaccaggagcgccggtagtctgtcctgaagaactggtgcgcttccttctattagacaacgagaactct | |
| gtccgttcttgtttcctttttgccgtgtttgccaggaagctcatttggtaaacgtcagtctcgtccctggtttctgacgtaattacctcccgt | |
| gttaccaggacggtttctggtttctccctcatttactttaaccaccctatgcccacgcgtttggtaaaaccactctcgacctgacttataaa | |
| gaaagcaggggagggaggtgtgacgtggtgtgagagccgggccccgggttcccactggcctccctgggtcagccaggctgccctggg | |
| cctgtcctggccatcaggcccctagggttgagcagaaggggaggtgctggcgaggcagaatctgctggagcggggacccaccaat | |
| gccctccactcagcccccgcctgcccacccccttgcagctcctgcttcaacggtggcacctgcgtggacggcatcaactcgttcacct | |
| gcctgtgtccacccggcttcacgggcagctactgccagcacgatgtcaatgagtgcgactcacagccctgcctgcatggcggcacct | |
| gtcaggacggctgcggctcctacaggtgcacctgcccccagggctacactggccccaactgccaggtgagtgcgccggccacaga | |
| ggtgcccgaaggaggggccctgggtgggtgcctacctgcgggaggtgggaacggtcaccccaggtcccactgtgtcggctcggg | |
| gtcacccccacacccccgggcagagggtttctggggatctgagcagccggtgaaggaacctgatgcggaagcagcaggcaccttc | |
| gtttcaatcccaggtttctggagccggggcaggagctcaggaattggggaattgaggaaaagtgttccttctagcaaaggccgaggg | |
| tggtctggacctgctgagggccctgagggagacccagcccaggctgttcctggtgtgggggtggtggggagtccaggggcggca | |
| gtttccacttctgtagaatgggttgcagcctgggttggagtaggccccttggcagatgtgcgttctgagctaccggggaaatggccgg | |
| ggcgcgggcacccagctgaccccaatctgtccccagaaccttgtgcactggtgtgactcctcgccctgcaagaacggcggcaaatg | |
| ctggcagacccacacccagtaccgctgcgagtgccccagcggctggaccggcctttactgcgacgtgcccagcgtgtcctgtgagg | |
| tggctgcgcagcgacaaggtaacctgctgtgcccacccagctcgggtcccagcccatcaaggtcctctgtgggcctgggcctcacct | |
| gtctaccaccccatcccccgcaggtgttgacgttgcccgcctgtgccagcatggagggctctgtgtggacgcgggcaacacgcacc | |
| actgccgctgccaggcgggctacacaggcagctactgtgaggacctggtggacgagtgctcacccagcccctgccagaacgggg | |
| ccacctgcacggactacctgggcggctactcctgcaaggtgggggtccctcctagggtaagggttgtggccggcacgagtgttgcc | |
| acacaccaggccctggctgggagctggcccagtggagaaaactgaacctgataggcccatgcactgttcagtctcattaggggagg | |
| gctggggtaatcagggtagactgcctggaagaggtggcctgtggaaagcctgaaggagggtacctatactgagaagtgggatggg | |
| gttttcccttcccctagattgtgtctgggcttggccaacacctaccctgaggccctcacctctatcctatgggacggggtccacccaccc | |
| ccaacaggcagtgactccggtcaccgaggccccgccagggtctgttgggctgggtctctctccaggtctgacaggagcgaggggc | |
| ccgtggcttcgctggacctgagggcagctcatgtggccctgtcagccctcacagtggggtgtgggagcactgcatcctggcgccgg | |
| ctgagccgaagggcccctcgttctgtcgcctgcacagtgcgtggccggctaccacggggtgaactgctctgaggagatcgacgagt | |
| gcctctcccacccctgccagaacgggggcacctgcctcgacctccccaacacctacaagtgctcctgcccacggggcactcagggt | |
| aagggccgctgcacggagggctggtgttggccatccatggccagggcaggggcagggcaggcaccccgggaccggccagagg | |
| catccaggaaccagccagaaactcccattttcctgtgtgaggccaaggccgactcacagctgcccagggaaagggcccagagcgg | |
| gggtcccagtgggaagggcgtctccacggcacccttgacacctgcctctcccgagtgtccgtgcagccccagacctgagcgctcgt | |
| cttccgggacggacacgcggcacggcagggccggggtgtggcgggcttgggccactgacgaaacctggccccgcaggtgtgca | |
| ctgtgagatcaacgtggacgactgcaatccccccgttgaccccgtgtcccggagccccaagtgctttaacaacggcacctgcgtgga | |
| ccaggtgggcggctacagctgcacctgcccgccgggcttcgtgggtgagcgctgtgagggggatgtcaacgagtgcctgtccaatc | |
| cctgcgacgcccgtggcacccagaactgcgtgcagcgcgtcaatgacttccactgcgagtgccgtgctggtcacaccggtgggtgc | |
| cgcgcccaggcgggtggggcgtgtggggcagcagggtgagcctctcactgccctgctcttacccctagggcgccgctgcgagtcc | |
| gtcatcaatggctgcaaaggcaagccctgcaagaatgggggcacctgcgccgtggcctccaacaccgcccgcgggttcatctgcaa | |
| gtgccctgcggtaggtgcaggggtgcagggaggcaggggcccgccaggggagacacctggagaggtccacgtgggggcctcg | |
| gggcgcagaccgggcagtgatcctcccggccttcatcctcctcctcaccctgatgtcttttttttttttttagtttcaataaatgattttag | |
| agacattttagatttatagaaaaatggagcaggaagtagactccctggggtggctgtcccctgcacgcagttcccctggttagcagcttgcat | |
| caatttcactctggatttcagtgatacattgttaccaacagcagcccttcctgtacctggggcttgcccttggctttgtggttgtgggtttgg | |
| gctgaggtatgacatgcctgcccaccctcgcaggatcacggcagagagtccctgccctaaagtcctcagcactccaccagtttaccc | |
| cttcctccatctcccgaccccctggcacccccgatctttctctgttggtagaatgtgtgtaaagggttttgctgctgggggcaaggttgca | |
| ggccgcctcccaggttagaggagagcggtggcactgctggccgagggctgggtgtgaggtggcgggggggcggggggtggcc | |
| caccccgacaccgtcctgtcttccctctcgggcagggcttcgagggcgccacgtgtgagaatgacgctcgtacctgcggcagcctgc | |
| gctgcctcaacggcggcacatgcatctccggcccgcgcagccccacctgcctgtgcctgggccccttcacgggccccgaatgcca | |
| gttcccggccagcagcccctgcctgggcggcaacccctgctacaaccaggggacctgtgagcccacatccgagagccccttctacc | |
| gttgcctgtgccccgccaaattcaacgggctcttgtgccacatcctggactacagcttcgggggtggggccgggcgcgacatccccc | |
| cgccgctgatcgaggaggcgtgcgagctgcccgagtgccaggaggacgcgggcaacaaggtctgcagcctgcagtgcaacaac | |
| cacgcgtgcggctgggacggcggtgactgctccctcaacttcaatgacccctggaagaactgcacgcagtctctgcagtgctggaa | |
| gtacttcagtgacggccactgtgacagccagtgcaactcagccggctgcctcttcgacggctttgactgccagcgtgcggaaggcca | |
| gtgcaagtaaggctgcggggctcatggggctgagggaggacctgaacttggatgtggcctggcttgggcccggaggccagcatgc | |
| agttctaaggctctgctcagggggtgcagggacgtcccccgcggctggccagtgggctggaggcaccggacggcgggtgcgagg | |
| ccccccgaggaaggcggcctgagcgtgtcccgccccccacagccccctgtacgaccagtactgcaaggaccacttcagcgacgg | |
| gcactgcgaccagggctgcaacagcgcggagtgcgagtgggacgggctggactgtgcggagcatgtacccgagaggctggcgg | |
| ccggcacgctggtggtggtggtgctgatgccgccggagcagctgcgcaacagctccttccacttcctgcgggagctcagccgcgtg | |
| ctgcacaccaacgtggtcttcaagcgtgacgcacacggccagcagatgatcttcccctactacggccgcgaggaggagctgcgcaa | |
| gcaccccatcaagcgtgccgccgagggctgggccgcacctgacgccctgctgggccaggtgaaggcctcgctgctccctggtggc | |
| agcgagggtgggcggcggcggagggagctggaccccatggacgtccgcgggtgagtgagacccggcgcccacggtcaatccc | |
| cgcaactctcctgggccctccccgacggcctccctgcccctcacggccggcgccatggcaagcagtactctccccactttatggtaa | |
| aagagacggaggttccgagaggacctgggacttcagggcctgcgcaggcaaggagtagaggcagcattccagctcagggccctg | |
| accccacagccacacccttttcctgggccactgccttcccctggacaggcggcactcctgtgcccagtaggtgattttgagattgagct | |
| gtgccttaggcactggatactacactgattaaaactcagccctctgccaggcgaggaggctcacacctgtaatcccagcactttggga | |
| ggctgaggcaggcggagcccttgagcccaggagttcgagaccagtctgggcaacatagggagaccttgtctctgttttttttaaaaaa | |
| gtattaaaagaagtaaaaaacaaaacactcagctctccaggggttcccacagggctgaacagccccaccccagacaagaatgccgc | |
| ttggtcatggcgtctgcctggcttgggctggagaggaggcaggtggaggtcctgggagggggcattgctgggccttgcagttggag | |
| gaggagctggtgggggtggggggtcccacggtgggagaacacaggcaggagcagcttgagtgcagggggcaccccacaaggc | |
| tcccgcccatgcctactcgatctggggcagctggacccaggagccaggttggttgtgcccttcgtgtgcctgaccctggtgggtttgc | |
| ctgtcagttctgctggcttggagcaatcctggaggtcagaagcatcatctcacaggctggatgggatcctgctcacgggaacccagtc | |
| ctggggagcagagctcacccccagccagcctcaccacacagcccaccaccgctgcacccacccccacctcacgcctgtgcttcctg | |
| cagggcctggggatggctcctgggggaggactgggctcctggcacatactctgtcctgagatgaggaaacgtgtctgtggcaccag | |
| caggagccagagagggtgtcagggcgggccagggagagcgcgttggtgggtatctgggatgagccgtgatcagcactggccgg | |
| agtcggggggctggcaccagtcccctgcagggtagctgctgtcagacctggcttcccaccaccccaggctgcctcaccatgtcctga | |
| ctgtggcgtcatgggcctcagtgtcctgcggcagcatccctggccggtgggcgggggaggaggaagcctcgggtcccagcccctc | |
| tctgattgtccgcccagctccatcgtctacctggagattgacaaccggcagtgtgtgcaggcctcctcgcagtgcttccagagtgccac | |
| cgacgtggccgcattcctgggagcgctcgcctcgctgggcagcctcaacatcccctacaagatcgaggccgtgcagagtaagtgtg | |
| gccccatcccgggaacaggctctgcctgcagggggtgccatccccccgtgccccagacacgctggctgtttgtgccagttgctaccc | |
| acgggtgtgagcgttgccgtccgagttggggtagggcttttctggaattttctgaatggcactccgcccccacctgcggcggtcacag | |
| ctgccggtggagccacctgggaacgagtccagccacgggaaagtgggtgcctgcttctctccccaccctttcctcctgaattttctttgt | |
| tgggtattatttcaaaatcattacggctttttttaaagaaaaaaaaagagagagagagaagaattgatcggtgtcatgtgaagtgttgaag | |
| tttgtatcttgaaaatccctctaaatcctttgtcttaacagctcagtgcgagtgcagcgatttgaagttgactaatcctccttccttaaaggag | |
| aaaaaagtaaaagccgtctccagatagagtcggctggtgcaggagagaatttagcgatagtttgcaattctgattaatcgcgtagaaaa | |
| tgaccttattttggagggcgggatggaggagagtgggtgaggaggcgcccggacgcggagccagtccgccgccccccggccacc | |
| agcctgctgcgtagccgctgcctgatgtccgggcacctgcccctggcccccgtgcccgcaggtgagaccgtggagccgcccccgc | |
| cggcgcagctgcacttcatgtacgtggcggcggccgcctttgtgcttctgttcttcgtgggctgcggggtgctgctgtcccgcaagcg | |
| ccggcggcagcatggccagctctggttccctgagggcttcaaagtgtctgaggccagcaagaagaagcggcgggagcccctcgg | |
| cgaggactccgtgggcctcaagtgagcggacgccgcccctgcttctgggtccccggtgggaggtgggacctggccgagcatcctc | |
| accgggagtctgcccctcccgacccagggcccacctcccacgccaggcccgggccaggggtctctgggggcttcctagggagct | |
| cgctcagcctcacttctcgacccctcaccccccaggcccctgaagaacgcttcagacggtgccctcatggacgacaaccagaatga | |
| gtggggggacgaggacctggagaccaagaagttccgggtgagtcgcgaggctcccgggctcctgggctcccgggcacctgctgc | |
| cgggctgccctgacaggctctgctcactccctctatgtagttcgaggagcccgtggttctgcctgacctggacgaccagacagaccac | |
| cggcagtggactcagcagcacctggatgccgctgacctgcgcatgtctgccatggcccccacaccgccccagggtgaggttgacg | |
| ccgactgcatggacgtcaatgtccgcgggcctggtaagggtgccagcagccagggcttccctagccccgtggcccacctgcctctct | |
| cccctaagccccgaggctggggtacagttgatactctggaaacttagaattgggggtgagagctttcatccttggggtgtttcccattca | |
| gagtagacgtgggggggtcctggagtcctcctgttcttccaccaaccccttcctggggtgataccgcagggccactctgtccctgtaa | |
| attactcttttctgaaaccttcttgagacatggaaagcgttgattttctttttctttttttttttcttttttttttgtttttgagatggag | |
| tctcgctccgtcacccaggctggagtgcagtggcacgatctcggctccctgcaacctccacctctcgggttcaagcaattctcctgcctcag | |
| cctccccagtagctgggactacaggcgcctgccaccacacctggctaatttttgtatttttagcagcgatggggtttcaccatgttggccagg | |
| ctggtctcgaactcctgacctcaggtgatccgcccacctcggcctcccccagtgctaggatgacagcgtgagcctccgcatcctgctgaagc | |
| attaattttctaactgcatgcttctggggtcctctttttcctgggtggattttgggtgccaggtcttgggcaggtcaggaagcagttgcccg | |
| ccaggagtttaagctggattcggctctgtccactgagcagcccaggcggacttcagctgccccttggtggctgtgtgcacggggcca | |
| ccaagtgctgggtggtgcatccacaccgtggccccttgagcttggggctgctgcccccctccccctgggctgcagctggggaccgg | |
| ccctccagactgagcacccgtctctgcctctgcagatggcttcaccccgctcatgatcgcctcctgcagcgggggcggcctggagac | |
| gggcaacagcgaggaagaggaggacgcgccggccgtcatctccgacttcatctaccagggcgccagcctgcacaaccagacaga | |
| ccgcacgggcgagaccgccttgcacctggccgcccgctactcacgctctgatgccgccaagcgcctgctggaggccagcgcagat | |
| gccaacatccaggacaacatgggccgcaccccgctgcatgcggctgtgtctgccgacgcacaaggtgtcttccaggtaggcagtgg | |
| ctgcctgtgtgcccacctgccctcctcagggccgcctggtggtctggggcagtggccaggcttacgtggccctgggagcctgaccc | |
| cgagcacagctgagtccgggacaactggtgcctccacctgggaccttcgcagtcagcgaggtgcgagggggagggcgtcgggcc | |
| catctgtgttctccagggaagtcaggcagaggcgggtctggcaggaggcctgggggatctgctgagtgaggcagcacctccccac | |
| ccccagcaaaacaggctccatcagggttgtgggccttgctcaaggtccaggttccactgctgcagcccctcgcagccccgcccctcc | |
| ctcaaccttggctgccggcgtagcctgtggcagtgagaagcagggtttagaggctgccgctcggtgcctgcagacctagggctcag | |
| cttgccggtgagctcgtggcaagaatggatttagggatttggatgcctgggtctccagggagtgtccctggcaggggctgcctttgca | |
| gtcacccctgctgcgagtccccaggctccaggcggccctggagcaagcaggttcagatgggcacagcccggggagttaccacag | |
| agcttctcatttcctgatttcttagctcaggtgacacattgtcatctcgcagaaataaatgtaggtgagcagaaagagcgtggaagggag | |
| ccccgtgcgggtttggtgtgtgcctctctcagctgccttctttgcacagatgtggaagttcccaggtgctttgcaggaatcaggccaagtt | |
| gccttttgcacccgccagtgagcaggggccattcctcctgccagatgccaagacccggctgcattacaagggctgcccaccccctct | |
| ctgggcagagccggacactagctcggcggttctgagacggctgtagggccgtgagcccggcttcctgagtgccagctgtaaccgcc | |
| gttgggggcagggacttgacctctctgttttgtaggtggtcatggcagctaggaccacagtgaggattaaatgaggccacacgtggtc | |
| acgatggatcccactgtcaccaaggggcctgtctgtgggcagcacagcccctgcccccatccgggcccctcctcaggggcagccc | |
| catggcgtttcgtcttgcccggccgtgccgatctcaggagggtctcgtctgtgtccggaagacagtggggcttcccgtccaggcgttc | |
| gttctgggcaggaggcatcggtgtacgtctgcccagcacccgcctgagcctctccctgttgcccagatcctgatccggaaccgagcc | |
| acagacctggatgcccgcatgcatgatggcacgacgccactgatcctggctgcccgcctggccgtggagggcatgctggaggacc | |
| tcatcaactcacacgccgacgtcaacgccgtagatgacctgggtgagcccacgggggcacggctgctctgtcgtggggcgggacc | |
| gccacagggacgggtgggactgggttgcctccagctggtctccaacctaccccatctgcttctttcacgcaggcaagtccgccctgca | |
| ctgggccgccgccgtgaacaatgtggatgccgcagttgtgctcctgaagaacggggctaacaaagatatgcagaacaacagggtga | |
| gcgcgaggctgggatgccaggggagacgtgagggctgaatccacagagaagtgagggccaaacccacggggcgcagggacac | |
| aagggcctgacccacagggtctcgagggtcgctgggcccgcatgtgggccccacccgcatgatgcgggcacctcgttagtgctcct | |
| gccttccttcagcccccgtttatggagcccttccaagtgcaggtccagctgtcaggacacggcggctcgcccctcagacccagcccta | |
| ccctcctgggcggcagtcatggcggggcacacagcctggcgtggggaacgctgctgctgccactgctgtgtcaccgtcacctggac | |
| ctcaccttctgtccccagctgtcaccaggcgggggtggggatgggaatgtggtgacgcaggtggtgcagattgagccagaacacgc | |
| gtggcggcagctccctgcggggcggggcctcttggtgtgttcaccaaggccaaggacctcaaggctcagaggaagaagtctcagg | |
| actatatccaaggggagccacccccagccctcatcctggccctgcagctcctggccctgcagctgctgttggtttcccctgggtgctca | |
| gggcacaggtgcagacacccccacctccctgccgccagaacccccacccccgcccccaactcctgctgcccccttggcatgtcag | |
| gctcaggcgtctctccctcctgggtgagggcacacagcgggcctgggcaccggggcatgttggcccaggcgctccccagtccgtg | |
| gcagcatggccccacagagactgggccccagagggcatcaaggcctgggaagccccttcccactcccacacagcagcctcaccc | |
| agacctgtgacgtgtccaccgcacagaggagaccccaggaaggagcttggtggccaccagggtggcttgatggccgtccagatga | |
| cgagctccctccctggtgccctcgccggtgcctctgaaccgctccaggaatttccttttgtgccttattgggggcagggaagagggcct | |
| gcagttggttagattttcagtggggtctgtgacccccccatagaggtggagccccgctgatctagggtagaggactgcacagatcccc | |
| tctctgggtgggtttcagaagatgtatcaaagccttaacatttaacaagagtcaggctaggtggttgcaggacgctggggtggggtcct | |
| gaggagcagcctgcctgcccccaccccgcggaggaggttgtactgctgcttcctctggtgatggaaccttggggagggtccccacg | |
| cctggcctggcccccctcaccggcccccgccctcatcccccaggaggagacacccctgtttctggccgcccgggagggcagctac | |
| gagaccgccaaggtgctgctggaccactttgccaaccgggacatcacggatcatatggaccgcctgccgcgcgacatcgcacagg | |
| agcgcatgcatcacgacatcgtgaggctgctggacgagtacaacctggtgcgcagcccgcagctgcacggagccccgctggggg | |
| gcacgcccaccctgtcgcccccgctctgctcgcccaacggctacctgggcagcctcaagcccggcgtgcagggcaagaaggtcc | |
| gcaagcccagcagcaaaggcctggcctgtggaagcaaggaggccaaggacctcaaggcacggaggaagaagtcccaggacgg | |
| caagggctgcctgctggacagctccggcatgctctcgcccgtggactccctggagtcaccccatggctacctgtcagacgtggcctc | |
| gccgccactgctgccctccccgttccagcagtctccgtccgtgcccctcaaccacctgcctgggatgcccgacacccacctgggcat | |
| cgggcacctgaacgtggcggccaagcccgagatggcggcgctgggtgggggcggccggctggcctttgagactggcccacctc | |
| gtctctcccacctgcctgtggcctctggcaccagcaccgtcctgggctccagcagcggaggggccctgaatttcactgtgggcgggt | |
| ccaccagtttgaatggtcaatgcgagtggctgtcccggctgcagagcggcatggtgccgaaccaatacaaccctctgcgggggagt | |
| gtggcaccaggccccctgagcacacaggccccctccctgcagcatggcatggtaggcccgctgcacagtagccttgctgccagcg | |
| ccctgtcccagatgatgagctaccagggcctgcccagcacccggctggccacccagcctcacctggtgcagacccagcaggtgca | |
| gccacaaaacttacagatgcagcagcagaacctgcagccagcaaacatccagcagcagcaaagcctgcagccgccaccaccacc | |
| accacagccgcaccttggcgtgagctcagcagccagcggccacctgggccggagcttcctgagtggagagccgagccaggcaga | |
| cgtgcagccactgggccccagcagcctggcggtgcacactattctgccccaggagagccccgccctgcccacgtcgctgccatcct | |
| cgctggtcccacccgtgaccgcagcccagttcctgacgcccccctcgcagcacagctactcctcgcctgtggacaacacccccagc | |
| caccagctacaggtgcctgagcaccccttcctcaccccgtcccctgagtcccctgaccagtggtccagctcgtccccgcattccaacg | |
| tctccgactggtccgagggcgtctccagccctcccaccagcatgcagtcccagatcgcccgcattccggaggccttcaagtaaacgg | |
| cgcgccccacgagaccccggcttcctttcccaagccttcgggcgtctgtgtgcgctctgtggatgccagggccgaccagaggagcc | |
| tttttaaaacacatgtttttatacaaaataagaacgaggattttaattttttttagtatttatttatgtacttttattttacacagaaaca | |
| ctgcctttttatttatatgtactgttttatctggccccaggtagaaacttttatctattctgagaaaacaagcaagttctgagagccaggg | |
| ttttcctacgtaggatgaaaagattcttctgtgtttataaaatataaacaaagattcatgatttataaatgccatttatttattgattcctt | |
| ttttcaaaatccaaaaagaaatgatgttggagaagggaagttgaacgagcatagtccaaaaagctcctggggcgtccaggccgcgcccttt | |
| ccccgacgcccacccaaccccaagccagcccggccgctccaccagcatcacctgcctgttaggagaagctgcatccagaggcaaacggaggc | |
| aaagctggctcaccttccgcacgcggattaatttgcatctgaaataggaaacaagtgaaagcatatgggttagatgttgccatgtgttttag | |
| atggtttcttgcaagcatgcttgtgaaaatgtgttctcggagtgtgtatgccaagagtgcacccatggtaccaatcatgaatctttgtttca | |
| ggttcagtattatgtagttgttcgttggttatacaagttcttggtccctccagaaccaccccggccccctgcccgttcttgaaatgtaggca | |
| tcatgcatgtcaaacatgagatgtgtggactgtggcacttgcctgggtcacacacggaggcatcctacccttttctggggaaagacactgcct | |
| gggctgaccccggtggcggccccagcacctcagcctgcacagtgtcccccaggttccgaagaagatgctccagcaacacagcctg | |
| ggccccagctcgcgggacccgaccccccgtgggctcccgtgttttgtaggagacttgccagagccgggcacattgagctgtgcaac | |
| gccgtgggctgcgtcctttggtcctgtccccgcagccctggcagggggcatgcggtcgggcaggggctggagggaggcgggggc | |
| tgcccttgggccacccctcctagtttgggaggagcagatttttgcaataccaagtatagcctatggcagaaaaaatgtctgtaaatatgtt | |
| tttaaaggtggattttgtttaaaaaatcttaatgaatgagtctgttgtgtgtcatgccagtgagggacgtcagacttggctcagctcgggga | |
| gccttagccgcccatgcactggggacgctccgctgccgtgccgcctgcactcctcagggcagcctcccccggctctacgggggcc | |
| gcgtggtgccatccccagggggcatgaccagatgcgtcccaagatgttgatttttactgtgttttataaaatagagtgtagtttacagaaa | |
| aagactttaaaagtgatctacatgaggaactgtagatgatgtatttttttcatcttttttgttaactgatttgcaataaaaatgatactgat | |
| ggtgatctggcttccactcccctctgctctggcctttggctccctttctgggagggaggcagggctgctatgctctgagggagccaggagtcg | |
| agggccccttctgctgggagagtgacggtgaggctgcctagtcctggcccacgggggtgtggggaccacgctgcctccagggact | |
| ccatggtgtctgcagcctgcctggtccaggccctttgtagggagatggacacacagcagcaagggggttgcagccctatgggaggt | |
| ggggctcgtgcctggggtgacacggctcccaggacaccatgcgtgagtctggccctcctggcagctcggggctcttctcctctcagc | |
| ctcggagggatgaaggtcccacccagccatcctggggacagcccctcagggagctctgcagcaggcagggggctgcatagggag | |
| ggccttgaggcagtggcatgacccctctacgggctggagaccacagggaggactctggcccctataggagggcaaagggagctgt | |
| cgaagcctatgagcagggcagcatgggtcgtggcagagtctggaaagttgtgtgatcctaaggaactggctgagcgaggcagagg | |
| cggcctggtcc | |
| Protein sequence: | SEQ ID |
| MPPLLAPLLCLALLPALAARGPRCSQPGETCLNGGKCEAANGTEACVCGGAFVGPR | NO: 349 |
| CQDPNPCLSTPCKNAGTCHVVDRRGVADYACSCALGFSGPLCLTPLDNACLTNPCR | |
| NGGTCDLLTLTEYKCRCPPGWSGKSCQQADPCASNPCANGGQCLPFEASYICHCPPS | |
| FHGPTCRQDVNECGQKPGLCRHGGTCHNEVGSYRCVCRATHTGPNCERPYVPCSPS | |
| PCQNGGTCRPTGDVTHECACLPGFTGQNCEENIDDCPGNNCKNGGACVDGVNTYN | |
| CRCPPEWTGQYCTEDVDECQLMPNACQNGGTCHNTHGGYNCVCVNGWTGEDCSE | |
| NIDDCASAACFHGATCHDRVASFYCECPHGRTGLLCHLNDACISNPCNEGSNCDTNP | |
| VNGKAICTCPSGYTGPACSQDVDECSLGANPCEHAGKCINTLGSFECQCLQGYTGPR | |
| CEIDVNECVSNPCQNDATCLDQIGEFQCICMPGYEGVHCEVNTDECASSPCLHNGRC | |
| LDKINEFQCECPTGFTGHLCQYDVDECASTPCKNGAKCLDGPNTYTCVCTEGYTGT | |
| HCEVDIDECDPDPCHYGSCKDGVATFTCLCRPGYTGHHCETNINECSSQPCRHGGTC | |
| QDRDNAYLCFCLKGTTGPNCEINLDDCAS SPCD SGTCLDKIDGYECACEPGYTGSMC | |
| NINIDECAGNPCHNGGTCEDGINGFTCRCPEGYHDPTCLSEVNECNSNPCVHGACRD | |
| SLNGYKCDCDPGWSGTNCDINNNECESNPCVNGGTCKDMTSGYVCTCREGFSGPN | |
| CQTNINECASNPCLNQGTCIDDVAGYKCNCLLPYTGATCEVVLAPCAPSPCRNGGEC | |
| RQSEDYESFSCVCPTGWQGQTCEVDINECVLSPCRHGASCQNTHGGYRCHCQAGYS | |
| GRNCETDIDDCRPNPCHNGGSCTDGINTAFCDCLPGFRGTFCEEDINECASDPCRNG | |
| ANCTDCVDSYTCTCPAGFSGIHCENNTPDCTESSCFNGGTCVDGINSFTCLCPPGFTG | |
| SYCQHDVNECDSQPCLHGGTCQDGCGSYRCTCPQGYTGPNCQNLVHWCDSSPCKN | |
| GGKCWQTHTQYRCECPSGWTGLYCDVPSVSCEVAAQRQGVDVARLCQHGGLCVD | |
| AGNTHHCRCQAGYTGSYCEDLVDECSPSPCQNGATCTDYLGGYSCKCVAGYHGVN | |
| CSEEIDECLSHPCQNGGTCLDLPNTYKCSCPRGTQGVHCEINVDDCNPPVDPVSRSP | |
| KCFNNGTCVDQVGGYSCTCPPGFVGERCEGDVNECLSNPCDARGTQNCVQRVNDF | |
| HCECRAGHTGRRCESVINGCKGKPCKNGGTCAVASNTARGFICKCPAGFEGATCEN | |
| DARTCGSLRCLNGGTCISGPRSPTCLCLGPFTGPECQFPASSPCLGGNPCYNQGTCEP | |
| TSESPFYRCLCPAKFNGLLCHILDYSFGGGAGRDIPPPLIEEACELPECQEDAGNKVCS | |
| LQCNNHACGWDGGDCSLNFNDPWKNCTQSLQCWKYFSDGHCDSQCNSAGCLFDG | |
| FDCQRAEGQCNPLYDQYCKDHFSDGHCDQGCNSAECEWDGLDCAEHVPERLAAGT | |
| LVVVVLMPPEQLRNSSFHFLRELSRVLHTNVVFKRDAHGQQMIFPYYGREEELRKH | |
| PIKRAAEGWAAPDALLGQVKASLLPGGSEGGRRRRELDPMDVRGSIVYLEIDNRQC | |
| VQASSQCFQSATDVAAFLGALASLGSLNIPYKIEAVQSETVEPPPPAQLHFMYVAAA | |
| AFVLLFFVGCGVLLSRKRRRQHGQLWFPEGFKVSEASKKKRREPLGEDSVGLKPLK | |
| NASDGALMDDNQNEWGDEDLETKKFRFEEPVVLPDLDDQTDHRQWTQQHLDAAD | |
| LRMSAMAPTPPQGEVDADCMDVNVRGPDGFTPLMIASCSGGGLETGNSEEEEDAPA | |
| VISDFIYQGASLHNQTDRTGETALHLAARYSRSDAAKRLLEASADANIQDNMGRTPL | |
| HAAVSADAQGVFQILIRNRATDLDARMHDGTTPLILAARLAVEGMLEDLINSHADV | |
| NAVDDLGKSALHWAAAVNNVDAAVVLLKNGANKDMQNNREETPLFLAAREGSYE | |
| TAKVLLDHFANRDITDHMDRLPRDIAQERMHHDIVRLLDEYNLVRSPQLHGAPLGG | |
| TPTLSPPLCSPNGYLGSLKPGVQGKKVRKPSSKGLACGSKEAKDLKARRKKSQDGK | |
| GCLLDSSGMLSPVDSLESPHGYLSDVASPPLLPSPFQQSPSVPLNHLPGMPDTHLGIG | |
| HLNVAAKPEMAALGGGGRLAFETGPPRLSHLPVASGTSTVLGSSSGGALNFTVGGS | |
| TSLNGQCEWLSRLQSGMVPNQYNPLRGSVAPGPLSTQAPSLQHGMVGPLHSSLAAS | |
| ALSQMMSYQGLPSTRLATQPHLVQTQQVQPQNLQMQQQNLQPANIQQQQSLQPPPP | |
| PPQPHLGVSSAASGHLGRSFLSGEPSQADVQPLGPSSLAVHTILPQESPALPTSLPSSL | |
| VPPVTAAQFLTPPSQHSYSSPVDNTPSHQLQVPEHPFLTPSPESPDQWSSSSPHSNVSD | |
| WSEGVSSPPTSMQSQIARIPEAFK | |
| CCR5 | SEQ ID |
| gaaaatccccactaagatcctgggtccagaaaaagatgggaaacctgtttagctcacccgtgagcccatagttaaaactctttagacaa | NO: 310 |
| caggttgtttccgtttacagagaacaataatattgggtggtgagcatctgtgtgggggttggggtgggataggggatacggggagagt | |
| ggagaaaaaggggacacagggttaatgtgaagtccaggatccccctctacatttaaagttggtttaagttggctttaattaatagcaact | |
| cttaagataatcagaattttcttaaccttttagccttactgttgaaaagccctgtgatcttgtacaaatcatttgcttcttggatagtaatt | |
| tcttttactaaaatgtgggcttttgactagatgaatgtaaatgttcttctagctctgatatcctttattctttatattttctaacagattctg | |
| tgtagtgggatgagcagagaacaaaaacaaaataatccagtgagaaaagcccgtaaataaaccttcagaccagagatctattctctagcttat | |
| tttaagctcaacttaaaaagaagaactgttctctgattcttttcgccttcaatacacttaatgatttaactccaccctccttcaaaagaaaca | |
| gcatttcctacttttatactgtctatatgattgatttgcacagctcatctggccagaagagctgagacatccgttcccctacaagaaactctc | |
| cccggtaagtaacctctcagctgcttggcctgttagttagcttctgagatgagtaaaagactttacaggaaacccatagaagacatttggca | |
| aacaccaagtgctcatacaattatcttaaaatataatctttaagataaggaaagggtcacagtttggaatgagtttcagacggttataacat | |
| caaagatacaaaacatgattgtgagtgaaagactttaaagggagcaatagtattttaataactaacaatccttacctctcaaaagaaagatt | |
| tgcagagagatgagtcttagctgaaatcttgaaatcttatcttctgctaaggagaactaaaccctctccagtgagatgccttctgaatatgt | |
| gcccacaagaagttgtgtctaagtctggttctcttttttctttttcctccagacaagagggaagcctaaaaatggtcaaaattaatattaaa | |
| ttacaaacgccaaataaaattttcctctaatatatcagtttcatggcacagttagtatataattctttatggttcaaaattaaaaatgagct | |
| tttctaggggcttctctcagctgcctagtctaaggtgcagggagtttgagactcacagggtttaataagagaaaattctcagctagagcagct | |
| gaacttaaatagactaggcaagacagctggttataagactaaactacccagaatgcatgacattcatctgtggtggcagacgaaacatttttt | |
| attatattatttcttgggtatgtatgacaactcttaattgtggcaactcagaaactacaaacacaaacttcacagaaaatgtgaggattttac | |
| aattggctgttgtcatctatgaccttccctgggacttgggcacccggccatttcactctgactacatcatgtcaccaaacatctgatggtctt | |
| gccttttaattctcttttcgaggactgagagggagggtagcatggtagttaagagtgcaggcttcccgcattcaaaatcggttgcttactagct | |
| gtgtggctttgagcaagttactcaccctctctgtgcttcaaggtccttgtctgcaaaatgtgaaaaatatttcctgcctcataaggttgccct | |
| aaggattaaatgaatgaatgggtatgatgcttagaacagtgattggcatccagtatgtgccctcgaggcctcttaattattactggcttgct | |
| catagtgcatgttctttgtgggctaactctagcgtcaataaaaatgttaagactgagttgcagccgggcatggtggctcatgcctgtaatc | |
| ccagcattctaggaggctgaggcaggaggatcgcttgagcccaggagttcgagaccagcctgggcaacatagtgtgatcttgtatct | |
| ataaaaataaacaaaattagcttggtgtggtggcgcctgtagtccccagccacttggaggggtgaggtgagaggattgcttgagcccg | |
| ggatggtccaggctgcagtgagccatgatcgtgccactgcactccagcctgggcgacagagtgagaccctgtctcacaacaacaac | |
| aacaacaacaaaaaggctgagctgcaccatgcttgacccagtttcttaaaattgttgtcaaagcttcattcactccatggtgctatagagc | |
| acaagattttatttggtgagatggtgctttcatgaattcccccaacagagccaagctctccatctagtggacagggaagctagcagcaa | |
| accttcccttcactacaaaacttcattgcttggccaaaaagagagttaattcaatgtagacatctatgtaggcaattaaaaacctattgatgt | |
| ataaaacagtttgcattcatggagggcaactaaatacattctaggactttataaaagatcactttttatttatgcacagggtggaacaagat | |
| ggattatcaagtgtcaagtccaatctatgacatcaattattatacatcggagccctgccaaaaaatcaatgtgaagcaaatcgcagcccg | |
| cctcctgcctccgctctactcactggtgttcatctttggttttgtgggcaacatgctggtcatcctcatcctgataaactgcaaaaggctga | |
| agagcatgactgacatctacctgctcaacctggccatctctgacctgtttttccttcttactgtccccttctgggctcactatgctgccgccc | |
| agtgggactttggaaatacaatgtgtcaactcttgacagggctctattttataggcttcttctctggaatcttcttcatcatcctcctgacaat | |
| cgataggtacctggctgtcgtccatgctgtgtttgctttaaaagccaggacggtcacctttggggtggtgacaagtgtgatcacttgggt | |
| ggtggctgtgtttgcgtctctcccaggaatcatctttaccagatctcaaaaagaaggtcttcattacacctgcagctctcattttccatacag | |
| tcagtatcaattctggaagaatttccagacattaaagatagtcatcttggggctggtcctgccgctgcttgtcatggtcatctgctactcgg | |
| gaatcctaaaaactctgcttcggtgtcgaaatgagaagaagaggcacagggctgtgaggcttatcttcaccatcatgattgtttattttctc | |
| ttctgggctccctacaacattgtccttctcctgaacaccttccaggaattctttggcctgaataattgcagtagctctaacaggttggacca | |
| agctatgcaggtgacagagactcttgggatgacgcactgctgcatcaaccccatcatctatgcctttgtcggggagaagttcagaaact | |
| acctcttagtcttcttccaaaagcacattgccaaacgcttctgcaaatgctgttctattttccagcaagaggctcccgagcgagcaagctc | |
| agtttacacccgatccactggggagcaggaaatatctgtgggcttgtgacacggactcaagtgggctggtgacccagtcagagttgtg | |
| cacatggcttagttttcatacacagcctgggctgggggtggggtgggagaggtcttttttaaaaggaagttactgttatagagggtctaa | |
| gattcatccatttatttggcatctgtttaaagtagattagatcttttaagcccatcaattatagaaagccaaatcaaaatatgttgatgaaaaat | |
| agcaacctttttatctccccttcacatgcatcaagttattgacaaactctcccttcactccgaaagttccttatgtatatttaaaagaaagcct | |
| cagagaattgctgattcttgagtttagtgatctgaacagaaataccaaaattatttcagaaatgtacaactttttacctagtacaaggcaac | |
| atataggttgtaaatgtgtttaaaacaggtctttgtcttgctatggggagaaaagacatgaatatgattagtaaagaaatgacacttttcatg | |
| tgtgatttcccctccaaggtatggttaataagtttcactgacttagaaccaggcgagagacttgtggcctgggagagctggggaagctt | |
| cttaaatgagaaggaatttgagttggatcatctattgctggcaaagacagaagcctcactgcaagcactgcatgggcaagcttggctgt | |
| agaaggagacagagctggttgggaagacatggggaggaaggacaaggctagatcatgaagaaccttgacggcattgctccgtcta | |
| agtcatgagctgagcagggagatcctggttggtgttgcagaaggtttactctgtggccaaaggagggtcaggaaggatgagcatttag | |
| ggcaaggagaccaccaacagccctcaggtcagggtgaggatggcctctgctaagctcaaggcgtgaggatgggaaggagggag | |
| gtattcgtaaggatgggaaggagggaggtattcgtgcagcatatgaggatgcagagtcagcagaactggggtggatttgggttggaa | |
| gtgagggtcagagaggagtcagagagaatccctagtcttcaagcagattggagaaacccttgaaaagacatcaagcacagaagga | |
| ggaggaggaggtttaggtcaagaagaagatggattggtgtaaaaggatgggtctggtttgcagagcttgaacacagtctcacccaga | |
| ctccaggctgtctttcactgaatgcttctgacttcatagatttccttcccatcccagctgaaatactgaggggtctccaggaggagactag | |
| atttatgaatacacgaggtatgaggtctaggaacatacttcagctcacacatgagatctaggtgaggattgattacctagtagtcatttcat | |
| gggttgttgggaggattctatgaggcaaccacaggcagcatttagcacatactacacattcaataagcatcaaactcttagttactcattc | |
| agggatagcactgagcaaagcattgagcaaaggggtcccatagaggtgagggaagcctgaaaaactaagatgctgcctgcccagt | |
| gcacacaagtgtaggtatcattttctgcatttaaccgtcaataggcaaaggggggaagggacatattcatttggaaataagctgccttga | |
| gccttaaaacccacaaaagtacaatttaccagcctccgtatttcagactgaatgggggtggggggggcgccttaggtacttattccaga | |
| tgccttctccagacaaaccagaagcaacagaaaaaatcgtctctccctccctttgaaatgaatataccccttagtgtttgggtatattcattt | |
| caaagggagagagagaggtttttttctgttctgtctcatatgattgtgcacatacttgagactgttttgaatttgggggatggctaaaaccat | |
| catagtacaggtaaggtgagggaatagtaagtggtgagaactactcagggaatgaaggtgtcagaataataagaggtgctactgactt | |
| tctcagcctctgaatatgaacggtgagcattgtggctgtcagcaggaagcaacgaagggaaatgtctttccttttgctcttaagttgtgga | |
| gagtgcaacagtagcataggaccctaccctctgggccaagtcaaagacattctgacatcttagtatttgcatattcttatgtatgtgaaagt | |
| tacaaattgcttgaaagaaaatatgcatctaataaaaaacaccttctaaaataattcattatattcttgctctttcagtcaagtgtacatttaga | |
| gaatagcacataaaactgccagagcattttataagcagctgttttcttccttagtgtgtgtgcatgtgtgtgtgatgtatacaaagagagag | |
| ataattgtatttttgtattttcttttaaataatttttaaaattgacccttttcctgagacaaattgccagaatagtttgtatttagagatggt | |
| acctctaagagtaaggttgctggttgctgagcaattgacttgaaaacttttaaaattcaaattttaattccactactcaaaagaattgccatg | |
| ttttaaaaaagagaattggtgccataagttagttgtctatgtttgaaaatgaagaagatatgcaacgtcatggcctggtcacttacccgcagc | |
| cctgagttgtaggcacatcatatgtgagaatgaggatgcttttctttcatttaaaatccctccccaaaacttggctctaattgcagtcatga | |
| caatcatgtacatttggatttatgtgcacgagtctcttaccctgagagaggacaggtgctacaggtggaggggacccgtctgggtcacgttcac | |
| at | |

Mutated gene sequences (variants detected after
genome sequencing, showing coding sequence
only)

| | |
|---|---|
| TP53 > P223fs | SEQ ID |
| atggaggagccgcagtcagatcctagcgtcgagcc | NO: 311 |
| ccctctgagtcaggaaacattttcagacctatgga | |
| aactacttcctgaaaacaacgttctgtcccccttg | |
| ccgtcccaagcaatggatgatttgatgctgtcccc | |
| ggacgatattgaacaatggttcactgaagacccag | |
| gtccagatgaagctcccagaatgccagaggctgct | |
| ccccccgtggcccctgcaccagcagctcctacacc | |
| ggcggcccctgcaccagccccctcctggcccctgt | |
| catcttctgtcccttcccagaaaacctaccagggc | |
| agctacggtttccgtctgggcttcttgcattctgg | |
| gacagccaagtctgtgacttgcacgtactcccctg | |
| ccctcaacaagatgttttgccaactggccaagacc | |
| tgccctgtgcagctgtgggttgattccacaccccc | |
| gcccggcacccgcgtccgcgccatggccatctaca | |
| agcagtcacagcacatgacggaggttgtgaggcgc | |
| tgcccccaccatgagcgctgctcagatagcgatgg | |
| tctggcccctcctcagcatcttatccgagtggaag | |
| gaaatttgcgtgtggagtatttggatgacagaaac | |
| acttttcgacatagtgtggtggtgccctatgagcc | |
| gcgaggttggctctgactgtaccaccatccactac | |
| aactacatgtgtaacagttcctgcatgggcggcat | |
| gaaccggaggcccatcctcaccatcatcacactgg | |
| aagactccagtggtaatctactgggacggaacagc | |
| tttgaggtgcgtgtttgtgcctgtcctgggagaga | |
| ccggcgcacagaggaagagaatctccgcaagaaag | |
| gggagcctcaccacgagctgcccccagggagcact | |
| aagcgagcactgcccaacaacaccagctcctctcc | |
| ccagccaaagaagaaaccactggatggagaatatt | |
| tcacccttcagatccgtgggcgtgagcgcttcgag | |
| atgttccgagagctgaatgaggccttggaactcaa | |
| ggatgcccaggctgggaaggagccaggggggagca | |
| gggctcactccagccacctgaagtccaaaaagggt | |
| cagtctacctcccgccataaaaaactcatgttcaa | |
| gacagaagggcctgactcagactga | |
| TP53 > 222_224del | SEQ ID |
| atggaggagccgcagtcagatcctagcgtcgagcc | NO: 312 |
| ccctctgagtcaggaaacattttcagacctatgga | |
| aactacttcctgaaaacaacgttctgtcccccttg | |
| ccgtcccaagcaatggatgatttgatgctgtcccc | |
| ggacgatattgaacaatggttcactgaagacccag | |
| gtccagatgaagctcccagaatgccagaggctgct | |
| ccccccgtggcccctgcaccagcagctcctacacc | |
| ggcggcccctgcaccagccccctcctggcccctgt | |
| catcttctgtcccttcccagaaaacctaccagggc | |
| agctacggtttccgtctgggcttcttgcattctgg | |
| gacagccaagtctgtgacttgcacgtactcccctg | |
| ccctcaacaagatgttttgccaactggccaagacc | |
| tgccctgtgcagctgtgggttgattccacaccccc | |
| gcccggcacccgcgtccgcgccatggccatctaca | |
| agcagtcacagcacatgacggaggttgtgaggcgc | |
| tgcccccaccatgagcgctgctcagatagcgatgg | |
| tctggcccctcctcagcatcttatccgagtggaag | |
| gaaatttgcgtgtggagtatttggatgacagaaac | |
| acttttcgacatagtgtggtggtgccctatgaggt | |
| tggctctgactgtaccaccatccactacaactaca | |
| tgtgtaacagttcctgcatgggcggcatgaaccgg | |
| aggcccatcctcaccatcatcacactggaagactc | |
| cagtggtaatctactgggacggaacagctttgagg | |
| tgcgtgtttgtgcctgtcctgggagagaccggcgc | |
| acagaggaagagaatctccgcaagaaaggggagcc | |
| tcaccacgagctgcccccagggagcactaagcgag | |
| cactgcccaacaacaccagctcctctccccagcca | |
| aagaagaaaccactggatggagaatatttcaccct | |
| tcagatccgtgggcgtgagcgcttcgagatgttcc | |
| gagagctgaatgaggccttggaactcaaggatgcc | |
| caggctgggaaggagccaggggggagcagggctca | |
| ctccagccacctgaagtccaaaaagggtcagtcta | |
| cctcccgccataaaaaactcatgttcaagacagaa | |
| gggcctgactcagactga | |
| CDKN2A > R46fs | SEQ ID |
| atggagccggcggcggggagcagcatggagccttc | NO: 313 |
| ggctgactggctggccacggccgcggcccggggtc | |
| gggtagaggaggtgcgggcgctgctggaggcgggg | |
| gcgctgcccaacgcaccgaatagttacggtcgagg | |
| ccgatccaggtcatgatgatgggcagcgcccgagt | |
| ggcggagctgctgctgctccacggcgcggagccca | |
| actgcgccgaccccgccactctcacccgacccgtg | |
| cacgacgctgcccgggagggcttcctggacacgct | |
| ggtggtgctgcaccgggccggggcgcggctggacg | |
| tgcgcgatgcctggggccgtctgcccgtggacctg | |
| gctgaggagctgggccatcgcgatgtcgcacggta | |
| cctgcgcgcggctgcggggggcaccagaggcagta | |
| accatgcccgcatagatgccgcggaaggtccctca | |
| gacatccccgattga | |
| CDKN2A > 44 45del | SEQ ID |
| atggagccggcggcggggagcagcatggagccttc | NO: 314 |
| ggctgactggctggccacggccgcggcccggggtc | |
| gggtagaggaggtgcgggcgctgctggaggcgggg | |
| gcgctgcccaacgcaccgaatagtcggaggccgat | |
| ccaggtcatgatgatgggcagcgcccgagtggcgg | |
| agctgctgctgctccacggcgcggagcccaactgc | |
| gccgaccccgccactctcacccgacccgtgcacga | |
| cgctgcccgggagggcttcctggacacgctggtgg | |
| tgctgcaccgggccggggcgcggctggacgtgcgc | |
| gatgcctggggccgtctgcccgtggacctggctga | |
| ggagctgggccatcgcgatgtcgcacggtacctgc | |
| gcgcggctgcggggggcaccagaggcagtaaccat | |
| gcccgcatagatgccgcggaaggtccctcagacat | |
| ccccgattga | |
| NOTCH1 > C292fs | SEQ ID |
| atgccgccgctcctggcgcccctgctctgcctggc | NO: 315 |
| gctgctgcccgcgctcgccgcacgaggcccgcgat | |
| gctcccagcccggtgagacctgcctgaatggcggg | |
| aagtgtgaagcggccaatggcacggaggcctgcgt | |
| ctgtggcggggccttcgtgggcccgcgatgccagg | |
| accccaacccgtgcctcagcaccccctgcaagaac | |
| gccgggacatgccacgtggtggaccgcagaggcgt | |
| ggcagactatgcctgcagctgtgccctgggcttct | |
| ctgggcccctctgcctgacacccctggacaatgcc | |
| tgcctcaccaacccctgccgcaacgggggcacctg | |
| cgacctgctcacgctgacggagtacaagtgccgct | |
| gcccgcccggctggtcagggaaatcgtgccagcag | |
| gctgacccgtgcgcctccaacccctgcgccaacgg | |
| tggccagtgcctgcccttcgaggcctcctacatct | |
| gccactgcccacccagcttccatggccccacctgc | |
| cggcaggatgtcaacgagtgtggccagaagcccgg | |
| gctttgccgccacggaggcacctgccacaacgagg | |
| tcggctcctaccgctgcgtctgccgcgccacccac | |
| actggccccaactgcgagcggccctacgtgccctg | |
| cagcccctcgccctgccagaacgggggcacctgcc | |
| gccccacgggcgacgtcacccacgagtgtgcctgc | |
| ctgccaggcttcaccggccagaactgtgaggaaaa | |
| tatcgacgattgtccaggaaacaactgcaagaacg | |
| ggggtgcctgtgtggacggcgtgaacacctacaac | |
| tgccgctgcccgccagagtggacaggtcagtacta | |
| ccgaggatgtggacgagtgccagctgatgccaaat | |
| gcctgccagaacggcgggacctgccacaacaccca | |
| cggtggctacaactgcgtgtgtgtcaacggctgga | |
| ctggtgaggactgcagcgagaacattgatgactgt | |
| gccagcgccgcctgcttccacggcgccacctgcca | |
| tgaccgtgtggcctccttctactgcgagtgtcccc | |
| atggccgcacaggtctgctgtgccacctcaacgac | |
| gcatgcatcagcaacccctgtaacgagggctccaa | |
| ctgcgacaccaaccctgtcaatggcaaggccatct | |
| gcacctgcccctcggggtacacgggcccggcctgc | |
| agccaggacgtggatgagtgctcgctgggtgccaa | |
| cccctgcgagcatgcgggcaagtgcatcaacacgc | |
| tgggctccttcgagtgccagtgtctgcagggctac | |
| acgggcccccgatgcgagatcgacgtcaacgagtg | |
| cgtctcgaacccgtgccagaacgacgccacctgcc | |
| tggaccagattggggagttccagtgcatctgcatg | |
| cccggctacgagggtgtgcactgcgaggtcaacac | |
| agacgagtgtgccagcagcccctgcctgcacaatg | |
| gccgctgcctggacaagatcaatgagttccagtgc | |
| gagtgccccacgggcttcactgggcatctgtgcca | |
| gtacgatgtggacgagtgtgccagcaccccctgca | |
| agaatggtgccaagtgcctggacggacccaacact | |
| tacacctgtgtgtgcacggaagggtacacggggac | |
| gcactgcgaggtggacatcgatgagtgcgaccccg | |
| acccctgccactacggctcctgcaaggacggcgtc | |
| gccaccttcacctgcctctgccgcccaggctacac | |
| gggccaccactgcgagaccaacatcaacgagtgct | |
| ccagccagccctgccgccacgggggcacctgccag | |
| gaccgcgacaacgcctacctctgcttctgcctgaa | |
| ggggaccacaggacccaactgcgagatcaacctgg | |
| atgactgtgccagcagcccctgcgactcgggcacc | |
| tgtctggacaagatcgatggctacgagtgtgcctg | |
| tgagccgggctacacagggagcatgtgtaacatca | |
| acatcgatgagtgtgcgggcaacccctgccacaac | |
| gggggcacctgcgaggacggcatcaatggcttcac | |
| ctgccgctgccccgagggctaccacgaccccacct | |
| gcctgtctgaggtcaatgagtgcaacagcaacccc | |
| tgcgtccacggggcctgccgggacagcctcaacgg | |
| gtacaagtgcgactgtgaccctgggtggagtggga | |
| ccaactgtgacatcaacaacaatgagtgtgaatcc | |
| aacccttgtgtcaacggcggcacctgcaaagacat | |
| gaccagtggctacgtgtgcacctgccgggagggct | |
| tcagcggtcccaactgccagaccaacatcaacgag | |
| tgtgcgtccaacccatgtctgaaccagggcacgtg | |
| tattgacgacgttgccgggtacaagtgcaactgcc | |
| tgctgccctacacaggtgccacgtgtgaggtggtg | |
| ctggccccgtgtgcccccagcccctgcagaaacgg | |
| cggggagtgcaggcaatccgaggactatgagagct | |
| tctcctgtgtctgccccacgggctggcaagggcag | |
| acctgtgaggtcgacatcaacgagtgcgttctgag | |
| cccgtgccggcacggcgcatcctgccagaacaccc | |
| acggcggctaccgctgccactgccaggccggctac | |
| agtgggcgcaactgcgagaccgacatcgacgactg | |
| ccggcccaacccgtgtcacaacgggggctcctgca | |
| cagacggcatcaacacggccttctgcgactgcctg | |
| cccggcttccggggcactttctgtgaggaggacat | |
| caacgagtgtgccagtgacccctgccgcaacgggg | |
| ccaactgcacggactgcgtggacagctacacgtgc | |
| acctgccccgcaggcttcagcgggatccactgtga | |
| gaacaacacgcctgactgcacagagagctcctgct | |
| tcaacggtggcacctgcgtggacggcatcaactcg | |
| ttcacctgcctgtgtccacccggcttcacgggcag | |
| ctactgccagcacgatgtcaatgagtgcgactcac | |
| agccctgcctgcatggcggcacctgtcaggacggc | |
| tgcggctcctacaggtgcacctgcccccagggcta | |
| cactggccccaactgccagaaccttgtgcactggt | |
| gtgactcctcgccctgcaagaacggcggcaaatgc | |
| tggcagacccacacccagtaccgctgcgagtgccc | |
| cagcggctggaccggcctttactgcgacgtgccca | |
| gcgtgtcctgtgaggtggctgcgcagcgacaaggt | |
| gttgacgttgcccgcctgtgccagcatggagggct | |
| ctgtgtggacgcgggcaacacgcaccactgccgct | |
| gccaggcgggctacacaggcagctactgtgaggac | |
| ctggtggacgagtgctcacccagcccctgccagaa | |
| cggggccacctgcacggactacctgggcggctact | |
| cctgcaagtgcgtggccggctaccacggggtgaac | |
| tgctctgaggagatcgacgagtgcctctcccaccc | |
| ctgccagaacgggggcacctgcctcgacctcccca | |
| acacctacaagtgctcctgcccacggggcactcag | |
| ggtgtgcactgtgagatcaacgtggacgactgcaa | |
| tccccccgttgaccccgtgtcccggagccccaagt | |
| gctttaacaacggcacctgcgtggaccaggtgggc | |
| ggctacagctgcacctgcccgccgggcttcgtggg | |
| tgagcgctgtgagggggatgtcaacgagtgcctgt | |
| ccaatccctgcgacgcccgtggcacccagaactgc | |
| gtgcagcgcgtcaatgacttccactgcgagtgccg | |
| tgctggtcacaccgggcgccgctgcgagtccgtca | |
| tcaatggctgcaaaggcaagccctgcaagaatggg | |
| ggcacctgcgccgtggcctccaacaccgcccgcgg | |
| gttcatctgcaagtgccctgcgggcttcgagggcg | |
| ccacgtgtgagaatgacgctcgtacctgcggcagc | |
| ctgcgctgcctcaacggcggcacatgcatctccgg | |
| cccgcgcagccccacctgcctgtgcctgggcccct | |
| tcacgggccccgaatgccagttcccggccagcagc | |
| ccctgcctgggcggcaacccctgctacaaccaggg | |
| gacctgtgagcccacatccgagagccccttctacc | |
| gttgcctgtgccccgccaaattcaacgggctcttg | |
| tgccacatcctggactacagcttcgggggtggggc | |
| cgggcgcgacatccccccgccgctgatcgaggagg | |
| cgtgcgagctgcccgagtgccaggaggacgcgggc | |
| aacaaggtctgcagcctgcagtgcaacaaccacgc | |
| gtgcggctgggacggcggtgactgctccctcaact | |
| tcaatgacccctggaagaactgcacgcagtctctg | |
| cagtgctggaagtacttcagtgacggccactgtga | |
| cagccagtgcaactcagccggctgcctcttcgacg | |
| gctttgactgccagcgtgcggaaggccagtgcaac | |
| cccctgtacgaccagtactgcaaggaccacttcag | |
| cgacgggcactgcgaccagggctgcaacagcgcgg | |
| agtgcgagtgggacgggctggactgtgcggagcat | |
| gtacccgagaggctggcggccggcacgctggtggt | |
| ggtggtgctgatgccgccggagcagctgcgcaaca | |
| gctccttccacttcctgcgggagctcagccgcgtg | |
| ctgcacaccaacgtggtcttcaagcgtgacgcaca | |
| cggccagcagatgatcttcccctactacggccgcg | |
| aggaggagctgcgcaagcaccccatcaagcgtgcc | |
| gccgagggctgggccgcacctgacgccctgctggg | |
| ccaggtgaaggcctcgctgctccctggtggcagcg | |
| agggtgggcggcggcggagggagctggaccccatg | |
| gacgtccgcggctccatcgtctacctggagattga | |
| caaccggcagtgtgtgcaggcctcctcgcagtgct | |
| tccagagtgccaccgacgtggccgcattcctggga | |
| gcgctcgcctcgctgggcagcctcaacatccccta | |
| caagatcgaggccgtgcagagtgagaccgtggagc | |
| cgcccccgccggcgcagctgcacttcatgtacgtg | |
| gcggcggccgcctttgtgcttctgttcttcgtggg | |
| ctgcggggtgctgctgtcccgcaagcgccggcggc | |
| agcatggccagctctggttccctgagggcttcaaa | |
| gtgtctgaggccagcaagaagaagcggcgggagcc | |
| cctcggcgaggactccgtgggcctcaagcccctga | |
| agaacgcttcagacggtgccctcatggacgacaac | |
| cagaatgagtggggggacgaggacctggagaccaa | |
| gaagttccggttcgaggagcccgtggttctgcctg | |
| acctggacgaccagacagaccaccggcagtggact | |
| cagcagcacctggatgccgctgacctgcgcatgtc | |
| tgccatggcccccacaccgccccagggtgaggttg | |
| acgccgactgcatggacgtcaatgtccgcgggcct | |
| gatggcttcaccccgctcatgatcgcctcctgcag | |
| cgggggcggcctggagacgggcaacagcgaggaag | |
| aggaggacgcgccggccgtcatctccgacttcatc | |
| taccagggcgccagcctgcacaaccagacagaccg | |
| cacgggcgagaccgccttgcacctggccgcccgct | |
| actcacgctctgatgccgccaagcgcctgctggag | |
| gccagcgcagatgccaacatccaggacaacatggg | |
| ccgcaccccgctgcatgcggctgtgtctgccgacg | |
| cacaaggtgtcttccagatcctgatccggaaccga | |
| gccacagacctggatgcccgcatgcatgatggcac | |
| gacgccactgatcctggctgcccgcctggccgtgg | |
| agggcatgctggaggacctcatcaactcacacgcc | |
| gacgtcaacgccgtagatgacctgggcaagtccgc | |
| cctgcactgggccgccgccgtgaacaatgtggatg | |
| ccgcagttgtgctcctgaagaacggggctaacaaa | |
| gatatgcagaacaacagggaggagacacccctgtt | |
| tctggccgcccgggagggcagctacgagaccgcca | |
| aggtgctgctggaccactttgccaaccgggacatc | |
| acggatcatatggaccgcctgccgcgcgacatcgc | |
| acaggagcgcatgcatcacgacatcgtgaggctgc | |
| tggacgagtacaacctggtgcgcagcccgcagctg | |
| cacggagccccgctggggggcacgcccaccctgtc | |
| gcccccgctctgctcgcccaacggctacctgggca | |
| gcctcaagcccggcgtgcagggcaagaaggtccgc | |
| aagcccagcagcaaaggcctggcctgtggaagcaa | |
| ggaggccaaggacctcaaggcacggaggaagaagt | |
| cccaggacggcaagggctgcctgctggacagctcc | |
| ggcatgctctcgcccgtggactccctggagtcacc | |
| ccatggctacctgtcagacgtggcctcgccgccac | |
| tgctgccctccccgttccagcagtctccgtccgtg | |
| cccctcaaccacctgcctgggatgcccgacaccca | |
| cctgggcatcgggcacctgaacgtggcggccaagc | |
| ccgagatggcggcgctgggtgggggcggccggctg | |
| gcctttgagactggcccacctcgtctctcccacct | |
| gcctgtggcctctggcaccagcaccgtcctgggct | |
| ccagcagcggaggggccctgaatttcactgtgggc | |
| gggtccaccagtttgaatggtcaatgcgagtggct | |
| gtcccggctgcagagcggcatggtgccgaaccaat | |
| acaaccctctgcgggggagtgtggcaccaggcccc | |
| ctgagcacacaggccccctccctgcagcatggcat | |
| ggtaggcccgctgcacagtagccttgctgccagcg | |
| ccctgtcccagatgatgagctaccagggcctgccc | |
| agcacccggctggccacccagcctcacctggtgca | |
| gacccagcaggtgcagccacaaaacttacagatgc | |
| agcagcagaacctgcagccagcaaacatccagcag | |
| cagcaaagcctgcagccgccaccaccaccaccaca | |
| gccgcaccttggcgtgagctcagcagccagcggcc | |
| acctgggccggagcttcctgagtggagagccgagc | |
| caggcagacgtgcagccactgggccccagcagcct | |
| ggcggtgcacactattctgccccaggagagccccg | |
| ccctgcccacgtcgctgccatcctcgctggtccca | |
| cccgtgaccgcagcccagttcctgacgcccccctc | |
| gcagcacagctactcctcgcctgtggacaacaccc | |
| ccagccaccagctacaggtgcctgagcaccccttc | |
| ctcaccccgtcccctgagtcccctgaccagtggtc | |
| cagctcgtccccgcattccaacgtctccgactggt | |
| ccgagggcgtctccagccctcccaccagcatgcag | |
| tcccagatcgcccgcattccggaggccttcaagta | |
| a | |
| NOTCHI > T293fs | SEQ ID |
| atgccgccgctcctggcgcccctgctctgcctggc | NO: 316 |
| gctgctgcccgcgctcgccgcacgaggcccgcgat | |
| gctcccagcccggtgagacctgcctgaatggcggg | |
| aagtgtgaagcggccaatggcacggaggcctgcgt | |
| ctgtggcggggccttcgtgggcccgcgatgccagg | |
| accccaacccgtgcctcagcaccccctgcaagaac | |
| gccgggacatgccacgtggtggaccgcagaggcgt | |
| ggcagactatgcctgcagctgtgccctgggcttct | |
| ctgggcccctctgcctgacacccctggacaatgcc | |
| tgcctcaccaacccctgccgcaacgggggcacctg | |
| cgacctgctcacgctgacggagtacaagtgccgct | |
| gcccgcccggctggtcagggaaatcgtgccagcag | |
| gctgacccgtgcgcctccaacccctgcgccaacgg | |
| tggccagtgcctgcccttcgaggcctcctacatct | |
| gccactgcccacccagcttccatggccccacctgc | |
| cggcaggatgtcaacgagtgtggccagaagcccgg | |
| gctttgccgccacggaggcacctgccacaacgagg | |
| tcggctcctaccgctgcgtctgccgcgccacccac | |
| actggccccaactgcgagcggccctacgtgccctg | |
| cagcccctcgccctgccagaacgggggcacctgcc | |
| gccccacgggcgacgtcacccacgagtgtgcctgc | |
| ctgccaggcttcaccggccagaactgtgaggaaaa | |
| tatcgacgattgtccaggaaacaactgcaagaacg | |
| ggggtgcctgtgtggacggcgtgaacacctacaac | |
| tgccgctgcccgccagagtggacaggtcagtactg | |
| tactcgaggatgtggacgagtgccagctgatgcca | |
| aatgcctgccagaacggcgggacctgccacaacac | |
| ccacggtggctacaactgcgtgtgtgtcaacggct | |
| ggactggtgaggactgcagcgagaacattgatgac | |
| tgtgccagcgccgcctgcttccacggcgccacctg | |
| ccatgaccgtgtggcctccttctactgcgagtgtc | |
| cccatggccgcacaggtctgctgtgccacctcaac | |
| gacgcatgcatcagcaacccctgtaacgagggctc | |
| caactgcgacaccaaccctgtcaatggcaaggcca | |
| tctgcacctgcccctcggggtacacgggcccggcc | |
| tgcagccaggacgtggatgagtgctcgctgggtgc | |
| caacccctgcgagcatgcgggcaagtgcatcaaca | |
| cgctgggctccttcgagtgccagtgtctgcagggc | |
| tacacgggcccccgatgcgagatcgacgtcaacga | |
| gtgcgtctcgaacccgtgccagaacgacgccacct | |
| gcctggaccagattggggagttccagtgcatctgc | |
| atgcccggctacgagggtgtgcactgcgaggtcaa | |
| cacagacgagtgtgccagcagcccctgcctgcaca | |
| atggccgctgcctggacaagatcaatgagttccag | |
| tgcgagtgccccacgggcttcactgggcatctgtg | |
| ccagtacgatgtggacgagtgtgccagcaccccct | |
| gcaagaatggtgccaagtgcctggacggacccaac | |
| acttacacctgtgtgtgcacggaagggtacacggg | |
| gacgcactgcgaggtggacatcgatgagtgcgacc | |
| ccgacccctgccactacggctcctgcaaggacggc | |
| gtcgccaccttcacctgcctctgccgcccaggcta | |
| cacgggccaccactgcgagaccaacatcaacgagt | |
| gctccagccagccctgccgccacgggggcacctgc | |
| caggaccgcgacaacgcctacctctgcttctgcct | |
| gaaggggaccacaggacccaactgcgagatcaacc | |
| tggatgactgtgccagcagcccctgcgactcgggc | |
| acctgtctggacaagatcgatggctacgagtgtgc | |
| ctgtgagccgggctacacagggagcatgtgtaaca | |
| tcaacatcgatgagtgtgcgggcaacccctgccac | |
| aacgggggcacctgcgaggacggcatcaatggctt | |
| cacctgccgctgccccgagggctaccacgacccca | |
| cctgcctgtctgaggtcaatgagtgcaacagcaac | |
| ccctgcgtccacggggcctgccgggacagcctcaa | |
| cgggtacaagtgcgactgtgaccctgggtggagtg | |
| ggaccaactgtgacatcaacaacaatgagtgtgaa | |
| tccaacccttgtgtcaacggcggcacctgcaaaga | |
| catgaccagtggctacgtgtgcacctgccgggagg | |
| gcttcagcggtcccaactgccagaccaacatcaac | |
| gagtgtgcgtccaacccatgtctgaaccagggcac | |
| gtgtattgacgacgttgccgggtacaagtgcaact | |
| gcctgctgccctacacaggtgccacgtgtgaggtg | |
| gtgctggccccgtgtgcccccagcccctgcagaaa | |
| cggcggggagtgcaggcaatccgaggactatgaga | |
| gcttctcctgtgtctgccccacgggctggcaaggg | |
| cagacctgtgaggtcgacatcaacgagtgcgttct | |
| gagcccgtgccggcacggcgcatcctgccagaaca | |
| cccacggcggctaccgctgccactgccaggccggc | |
| tacagtgggcgcaactgcgagaccgacatcgacga | |
| ctgccggcccaacccgtgtcacaacgggggctcct | |
| gcacagacggcatcaacacggccttctgcgactgc | |
| ctgcccggcttccggggcactttctgtgaggagga | |
| catcaacgagtgtgccagtgacccctgccgcaacg | |
| gggccaactgcacggactgcgtggacagctacacg | |
| tgcacctgccccgcaggcttcagcgggatccactg | |
| tgagaacaacacgcctgactgcacagagagctcct | |
| gcttcaacggtggcacctgcgtggacggcatcaac | |
| tcgttcacctgcctgtgtccacccggcttcacggg | |
| cagctactgccagcacgatgtcaatgagtgcgact | |
| cacagccctgcctgcatggcggcacctgtcaggac | |
| ggctgcggctcctacaggtgcacctgcccccaggg | |
| ctacactggccccaactgccagaaccttgtgcact | |
| ggtgtgactcctcgccctgcaagaacggcggcaaa | |
| tgctggcagacccacacccagtaccgctgcgagtg | |
| ccccagcggctggaccggcctttactgcgacgtgc | |
| ccagcgtgtcctgtgaggtggctgcgcagcgacaa | |
| ggtgttgacgttgcccgcctgtgccagcatggagg | |
| gctctgtgtggacgcgggcaacacgcaccactgcc | |
| gctgccaggcgggctacacaggcagctactgtgag | |
| gacctggtggacgagtgctcacccagcccctgcca | |
| gaacggggccacctgcacggactacctgggcggct | |
| actcctgcaagtgcgtggccggctaccacggggtg | |
| aactgctctgaggagatcgacgagtgcctctccca | |
| cccctgccagaacgggggcacctgcctcgacctcc | |
| ccaacacctacaagtgctcctgcccacggggcact | |
| cagggtgtgcactgtgagatcaacgtggacgactg | |
| caatccccccgttgaccccgtgtcccggagcccca | |
| agtgctttaacaacggcacctgcgtggaccaggtg | |
| ggcggctacagctgcacctgcccgccgggcttcgt | |
| gggtgagcgctgtgagggggatgtcaacgagtgcc | |
| tgtccaatccctgcgacgcccgtggcacccagaac | |
| tgcgtgcagcgcgtcaatgacttccactgcgagtg | |
| ccgtgctggtcacaccgggcgccgctgcgagtccg | |
| tcatcaatggctgcaaaggcaagccctgcaagaat | |
| gggggcacctgcgccgtggcctccaacaccgcccg | |
| cgggttcatctgcaagtgccctgcgggcttcgagg | |
| gcgccacgtgtgagaatgacgctcgtacctgcggc | |
| agcctgcgctgcctcaacggcggcacatgcatctc | |
| cggcccgcgcagccccacctgcctgtgcctgggcc | |
| ccttcacgggccccgaatgccagttcccggccagc | |
| agcccctgcctgggcggcaacccctgctacaacca | |
| ggggacctgtgagcccacatccgagagccccttct | |
| accgttgcctgtgccccgccaaattcaacgggctc | |
| ttgtgccacatcctggactacagcttcgggggtgg | |
| ggccgggcgcgacatccccccgccgctgatcgagg | |
| aggcgtgcgagctgcccgagtgccaggaggacgcg | |
| ggcaacaaggtctgcagcctgcagtgcaacaacca | |
| cgcgtgcggctgggacggcggtgactgctccctca | |
| acttcaatgacccctggaagaactgcacgcagtct | |
| ctgcagtgctggaagtacttcagtgacggccactg | |
| tgacagccagtgcaactcagccggctgcctcttcg | |
| acggctttgactgccagcgtgcggaaggccagtgc | |
| aaccccctgtacgaccagtactgcaaggaccactt | |
| cagcgacgggcactgcgaccagggctgcaacagcg | |
| cggagtgcgagtgggacgggctggactgtgcggag | |
| catgtacccgagaggctggcggccggcacgctggt | |
| ggtggtggtgctgatgccgccggagcagctgcgca | |
| acagctccttccacttcctgcgggagctcagccgc | |
| gtgctgcacaccaacgtggtcttcaagcgtgacgc | |
| acacggccagcagatgatcttcccctactacggcc | |
| gcgaggaggagctgcgcaagcaccccatcaagcgt | |
| gccgccgagggctgggccgcacctgacgccctgct | |
| gggccaggtgaaggcctcgctgctccctggtggca | |
| gcgagggtgggcggcggcggagggagctggacccc | |
| atggacgtccgcggctccatcgtctacctggagat | |
| tgacaaccggcagtgtgtgcaggcctcctcgcagt | |
| gcttccagagtgccaccgacgtggccgcattcctg | |
| ggagcgctcgcctcgctgggcagcctcaacatccc | |
| ctacaagatcgaggccgtgcagagtgagaccgtgg | |
| agccgcccccgccggcgcagctgcacttcatgtac | |
| gtggcggcggccgcctttgtgcttctgttcttcgt | |
| gggctgcggggtgctgctgtcccgcaagcgccggc | |
| ggcagcatggccagctctggttccctgagggcttc | |
| aaagtgtctgaggccagcaagaagaagcggcggga | |
| gcccctcggcgaggactccgtgggcctcaagcccc | |
| tgaagaacgcttcagacggtgccctcatggacgac | |
| aaccagaatgagtggggggacgaggacctggagac | |
| caagaagttccggttcgaggagcccgtggttctgc | |
| ctgacctggacgaccagacagaccaccggcagtgg | |
| actcagcagcacctggatgccgctgacctgcgcat | |
| gtctgccatggcccccacaccgccccagggtgagg | |
| ttgacgccgactgcatggacgtcaatgtccgcggg | |
| cctgatggcttcaccccgctcatgatcgcctcctg | |
| cagcgggggcggcctggagacgggcaacagcgagg | |
| aagaggaggacgcgccggccgtcatctccgacttc | |
| atctaccagggcgccagcctgcacaaccagacaga | |
| ccgcacgggcgagaccgccttgcacctggccgccc | |
| gctactcacgctctgatgccgccaagcgcctgctg | |
| gaggccagcgcagatgccaacatccaggacaacat | |
| gggccgcaccccgctgcatgcggctgtgtctgccg | |
| acgcacaaggtgtcttccagatcctgatccggaac | |
| cgagccacagacctggatgcccgcatgcatgatgg | |
| cacgacgccactgatcctggctgcccgcctggccg | |
| tggagggcatgctggaggacctcatcaactcacac | |
| gccgacgtcaacgccgtagatgacctgggcaagtc | |
| cgccctgcactgggccgccgccgtgaacaatgtgg | |
| atgccgcagttgtgctcctgaagaacggggctaac | |
| aaagatatgcagaacaacagggaggagacacccct | |
| gtttctggccgcccgggagggcagctacgagaccg | |
| ccaaggtgctgctggaccactttgccaaccgggac | |
| atcacggatcatatggaccgcctgccgcgcgacat | |
| cgcacaggagcgcatgcatcacgacatcgtgaggc | |
| tgctggacgagtacaacctggtgcgcagcccgcag | |
| ctgcacggagccccgctggggggcacgcccaccct | |
| gtcgcccccgctctgctcgcccaacggctacctgg | |
| gcagcctcaagcccggcgtgcagggcaagaaggtc | |
| cgcaagcccagcagcaaaggcctggcctgtggaag | |
| caaggaggccaaggacctcaaggcacggaggaaga | |
| agtcccaggacggcaagggctgcctgctggacagc | |
| tccggcatgctctcgcccgtggactccctggagtc | |
| accccatggctacctgtcagacgtggcctcgccgc | |
| cactgctgccctccccgttccagcagtctccgtcc | |
| gtgcccctcaaccacctgcctgggatgcccgacac | |
| ccacctgggcatcgggcacctgaacgtggcggcca | |
| agcccgagatggcggcgctgggtgggggcggccgg | |
| ctggcctttgagactggcccacctcgtctctccca | |
| cctgcctgtggcctctggcaccagcaccgtcctgg | |
| gctccagcagcggaggggccctgaatttcactgtg | |
| ggcgggtccaccagtttgaatggtcaatgcgagtg | |
| gctgtcccggctgcagagcggcatggtgccgaacc | |
| aatacaaccctctgcgggggagtgtggcaccaggc | |
| cccctgagcacacaggccccctccctgcagcatgg | |
| catggtaggcccgctgcacagtagccttgctgcca | |
| gcgccctgtcccagatgatgagctaccagggcctg | |
| cccagcacccggctggccacccagcctcacctggt | |
| gcagacccagcaggtgcagccacaaaacttacaga | |
| tgcagcagcagaacctgcagccagcaaacatccag | |
| cagcagcaaagcctgcagccgccaccaccaccacc | |
| acagccgcaccttggcgtgagctcagcagccagcg | |
| gccacctgggccggagcttcctgagtggagagccg | |
| agccaggcagacgtgcagccactgggccccagcag | |
| cctggcggtgcacactattctgccccaggagagcc | |
| ccgccctgcccacgtcgctgccatcctcgctggtc | |
| ccacccgtgaccgcagcccagttcctgacgccccc | |
| ctcgcagcacagctactcctcgcctgtggacaaca | |
| cccccagccaccagctacaggtgcctgagcacccc | |
| ttcctcaccccgtcccctgagtcccctgaccagtg | |
| gtccagctcgtccccgcattccaacgtctccgact | |
| ggtccgagggcgtctccagccctcccaccagcatg | |
| cagtcccagatcgcccgcattccggaggccttcaa | |
| gtaa | |

Representative coding sequences for targeting:

| | |
|---|---|
| Tp53 | SEQ ID |
| atggaggagccgcagtcagatcctagcgtcgagccccctctgagtcagga | NO: 324 |
| aacattttcagacctatggaaactacttcctgaaaacaacgttctgtccc | |
| ccttgccgtcccaagcaatggatgatttgatgctgtccccggacgatatt | |
| gaacaatggttcactgaagacccaggtccagatgaagctcccagaatgcc | |
| agaggctgctccccccgtggcccctgcaccagcagctcctacaccggcgg | |
| cccctgcaccagccccctcctggcccctgtcatcttctgtcccttcccag | |
| aaaacctaccagggcagctacggtttccgtctgggcttcttgcattctgg | |
| gacagccaagtctgtgacttgcacgtactcccctgccctcaacaagatgt | |
| tttgccaactggccaagacctgccctgtgcagctgtgggttgattccaca | |
| cccccgcccggcacccgcgtccgcgccatggccatctacaagcagtcaca | |
| gcacatgacggaggttgtgaggcgctgcccccaccatgagcgctgctcag | |
| atagcgatggtctggcccctcctcagcatcttatccgagtggaaggaaat | |
| ttgcgtgtggagtatttggatgacagaaacacttttcgacatagtgtggt | |
| ggtgccctatgagccgcctgaggttggctctgactgtaccaccatccact | |
| acaactacatgtgtaacagttcctgcatgggcggcatgaaccggaggccc | |
| atcctcaccatcatcacactggaagactccagtggtaatctactgggacg | |
| gaacagctttgaggtgcgtgtttgtgcctgtcctgggagagaccggcgca | |
| cagaggaagagaatctccgcaagaaaggggagcctcaccacgagctgccc | |
| ccagggagcactaagcgagcactgcccaacaacaccagctcctctcccca | |
| gccaaagaagaaaccactggatggagaatatttcacccttcaggaccaga | |
| ccagctttcaaaaagaaaattgttaa | |
| CDKN2A | SEQ ID |
| atggtgcgcaggttcttggtgaccctccggattcggcgcgcgtgcggccc | NO: 325 |
| gccgcgagtgagggttttcgtggttcacatcccgcggctcacgggggagt | |
| gggcagcgccaggggcgcccgccgctgtggccctcgtgctgatgctactg | |
| aggagccagcgtctagggcagcagccgcttcctagaagaccaggtcatga | |
| tgatgggcagcgcccgagtggcggagctgctgctgctccacggcgcggag | |
| cccaactgcgccgaccccgccactctcacccgacccgtgcacgacgctgc | |
| ccgggagggcttcctggacacgctggtggtgctgcaccgggccggggcgc | |
| ggctggacgtgcgcgatgcctggggccgtctgcccgtggacctggctga | |
| NOTCH1 | SEQ ID |
| atgccgccgctcctggcgcccctgctctgcctggcgctgctgcccgcgct | NO: 326 |
| cgccgcacgaggcccgcgatgctcccagcccggtgagacctgcctgaatg | |
| gcgggaagtgtgaagcggccaatggcacggaggcctgcgtctgtggcggg | |
| gccttcgtgggcccgcgatgccaggaccccaacccgtgcctcagcacccc | |
| ctgcaagaacgccgggacatgccacgtggtggaccgcagaggcgtggcag | |
| actatgcctgcagctgtgccctgggcttctctgggcccctctgcctgaca | |
| cccctggacaatgcctgcctcaccaacccctgccgcaacgggggcacctg | |
| cgacctgctcacgctgacggagtacaagtgccgctgcccgcccggctggt | |
| cagggaaatcgtgccagcaggctgacccgtgcgcctccaacccctgcgcc | |
| aacggtggccagtgcctgcccttcgaggcctcctacatctgccactgccc | |
| acccagcttccatggccccacctgccggcaggatgtcaacgagtgtggcc | |
| agaagcccgggctttgccgccacggaggcacctgccacaacgaggtcggc | |
| tcctaccgctgcgtctgccgcgccacccacactggccccaactgcgagcg | |
| gccctacgtgccctgcagcccctcgccctgccagaacgggggcacctgcc | |
| gccccacgggcgacgtcacccacgagtgtgcctgcctgccaggcttcacc | |
| ggccagaactgtgaggaaaatatcgacgattgtccaggaaacaactgcaa | |
| gaacgggggtgcctgtgtggacggcgtgaacacctacaactgccgctgcc | |
| cgccagagtggacaggtcagtactgtaccgaggatgtggacgagtgccag | |
| ctgatgccaaatgcctgccagaacggcgggacctgccacaacacccacgg | |
| tggctacaactgcgtgtgtgtcaacggctggactggtgaggactgcagcg | |
| agaacattgatgactgtgccagcgccgcctgcttccacggcgccacctgc | |
| catgaccgtgtggcctccttctactgcgagtgtccccatggccgcacagg | |
| tctgctgtgccacctcaacgacgcatgcatcagcaacccctgtaacgagg | |
| gctccaactgcgacaccaaccctgtcaatggcaaggccatctgcacctgc | |
| ccctcggggtacacgggcccggcctgcagccaggacgtggatgagtgctc | |
| gctgggtgccaacccctgcgagcatgcgggcaagtgcatcaacacgctgg | |
| gctccttcgagtgccagtgtctgcagggctacacgggcccccgatgcgag | |
| atcgacgtcaacgagtgcgtctcgaacccgtgccagaacgacgccacctg | |
| cctggaccagattggggagttccagtgcatctgcatgcccggctacgagg | |
| gtgtgcactgcgaggtcaacacagacgagtgtgccagcagcccctgcctg | |
| cacaatggccgctgcctggacaagatcaatgagttccagtgcgagtgccc | |
| cacgggcttcactgggcatctgtgccagtacgatgtggacgagtgtgcca | |
| gcaccccctgcaagaatggtgccaagtgcctggacggacccaacacttac | |
| acctgtgtgtgcacggaagggtacacggggacgcactgcgaggtggacat | |
| cgatgagtgcgaccccgacccctgccactacggctcctgcaaggacggcg | |
| tcgccaccttcacctgcctctgccgcccaggctacacgggccaccactgc | |
| gagaccaacatcaacgagtgctccagccagccctgccgccacgggggcac | |
| ctgccaggaccgcgacaacgcctacctctgcttctgcctgaaggggacca | |
| caggacccaactgcgagatcaacctggatgactgtgccagcagcccctgc | |
| gactcgggcacctgtctggacaagatcgatggctacgagtgtgcctgtga | |
| gccgggctacacagggagcatgtgtaacatcaacatcgatgagtgtgcgg | |
| gcaacccctgccacaacgggggcacctgcgaggacggcatcaatggcttc | |
| acctgccgctgccccgagggctaccacgaccccacctgcctgtctgaggt | |
| caatgagtgcaacagcaacccctgcgtccacggggcctgccgggacagcc | |
| tcaacgggtacaagtgcgactgtgaccctgggtggagtgggaccaactgt | |
| gacatcaacaacaatgagtgtgaatccaacccttgtgtcaacggcggcac | |
| ctgcaaagacatgaccagtggctacgtgtgcacctgccgggagggcttca | |
| gcggtcccaactgccagaccaacatcaacgagtgtgcgtccaacccatgt | |
| ctgaaccagggcacgtgtattgacgacgttgccgggtacaagtgcaactg | |
| cctgctgccctacacaggtgccacgtgtgaggtggtgctggccccgtgtg | |
| cccccagcccctgcagaaacggcggggagtgcaggcaatccgaggactat | |
| gagagcttctcctgtgtctgccccacgggctggcaagggcagacctgtga | |
| ggtcgacatcaacgagtgcgttctgagcccgtgccggcacggcgcatcct | |
| gccagaacacccacggcggctaccgctgccactgccaggccggctacagt | |
| gggcgcaactgcgagaccgacatcgacgactgccggcccaacccgtgtca | |
| caacgggggctcctgcacagacggcatcaacacggccttctgcgactgcc | |
| tgcccggcttccggggcactttctgtgaggaggacatcaacgagtgtgcc | |
| agtgacccctgccgcaacggggccaactgcacggactgcgtggacagcta | |
| cacgtgcacctgccccgcaggcttcagcgggatccactgtgagaacaaca | |
| cgcctgactgcacagagagctcctgcttcaacggtggcacctgcgtggac | |
| ggcatcaactcgttcacctgcctgtgtccacccggcttcacgggcagcta | |
| ctgccagcacgatgtcaatgagtgcgactcacagccctgcctgcatggcg | |
| gcacctgtcaggacggctgcggctcctacaggtgcacctgcccccagggc | |
| tacactggccccaactgccagaaccttgtgcactggtgtgactcctcgcc | |
| ctgcaagaacggcggcaaatgctggcagacccacacccagtaccgctgcg | |
| agtgccccagcggctggaccggcctttactgcgacgtgcccagcgtgtcc | |
| tgtgaggtggctgcgcagcgacaaggtgttgacgttgcccgcctgtgcca | |
| gcatggagggctctgtgtggacgcgggcaacacgcaccactgccgctgcc | |
| aggcgggctacacaggcagctactgtgaggacctggtggacgagtgctca | |
| cccagcccctgccagaacggggccacctgcacggactacctgggcggcta | |
| ctcctgcaagtgcgtggccggctaccacggggtgaactgctctgaggaga | |
| tcgacgagtgcctctcccacccctgccagaacgggggcacctgcctcgac | |
| ctccccaacacctacaagtgctcctgcccacggggcactcagggtgtgca | |
| ctgtgagatcaacgtggacgactgcaatccccccgttgaccccgtgtccc | |
| ggagccccaagtgctttaacaacggcacctgcgtggaccaggtgggcggc | |
| tacagctgcacctgcccgccgggcttcgtgggtgagcgctgtgaggggga | |
| tgtcaacgagtgcctgtccaatccctgcgacgcccgtggcacccagaact | |
| gcgtgcagcgcgtcaatgacttccactgcgagtgccgtgctggtcacacc | |
| gggcgccgctgcgagtccgtcatcaatggctgcaaaggcaagccctgcaa | |
| gaatgggggcacctgcgccgtggcctccaacaccgcccgcgggttcatct | |
| gcaagtgccctgcgggcttcgagggcgccacgtgtgagaatgacgctcgt | |
| acctgcggcagcctgcgctgcctcaacggcggcacatgcatctccggccc | |
| gcgcagccccacctgcctgtgcctgggccccttcacgggccccgaatgcc | |
| agttcccggccagcagcccctgcctgggcggcaacccctgctacaaccag | |
| gggacctgtgagcccacatccgagagccccttctaccgttgcctgtgccc | |
| cgccaaattcaacgggctcttgtgccacatcctggactacagcttcgggg | |
| gtggggccgggcgcgacatccccccgccgctgatcgaggaggcgtgcgag | |
| ctgcccgagtgccaggaggacgcgggcaacaaggtctgcagcctgcagtg | |
| caacaaccacgcgtgcggctgggacggcggtgactgctccctcaacttca | |
| atgacccctggaagaactgcacgcagtctctgcagtgctggaagtacttc | |
| agtgacggccactgtgacagccagtgcaactcagccggctgcctcttcga | |
| cggctttgactgccagcgtgcggaaggccagtgcaaccccctgtacgacc | |
| agtactgcaaggaccacttcagcgacgggcactgcgaccagggctgcaac | |
| agcgcggagtgcgagtgggacgggctggactgtgcggagcatgtacccga | |
| gaggctggcggccggcacgctggtggtggtggtgctgatgccgccggagc | |
| agctgcgcaacagctccttccacttcctgcgggagctcagccgcgtgctg | |
| cacaccaacgtggtcttcaagcgtgacgcacacggccagcagatgatctt | |
| cccctactacggccgcgaggaggagctgcgcaagcaccccatcaagcgtg | |
| ccgccgagggctgggccgcacctgacgccctgctgggccaggtgaaggcc | |
| tcgctgctccctggtggcagcgagggtgggcggcggcggagggagctgga | |
| ccccatggacgtccgcggctccatcgtctacctggagattgacaaccggc | |
| agtgtgtgcaggcctcctcgcagtgcttccagagtgccaccgacgtggcc | |
| gcattcctgggagcgctcgcctcgctgggcagcctcaacatcccctacaa | |
| gatcgaggccgtgcagagtgagaccgtggagccgcccccgccggcgcagc | |
| tgcacttcatgtacgtggcggcggccgcctttgtgcttctgttcttcgtg | |
| ggctgcggggtgctgctgtcccgcaagcgccggcggcagcatggccagct | |
| ctggttccctgagggcttcaaagtgtctgaggccagcaagaagaagcggc | |
| gggagcccctcggcgaggactccgtgggcctcaagcccctgaagaacgct | |
| tcagacggtgccctcatggacgacaaccagaatgagtggggggacgagga | |
| cctggagaccaagaagttccggttcgaggagcccgtggttctgcctgacc | |
| tggacgaccagacagaccaccggcagtggactcagcagcacctggatgcc | |
| gctgacctgcgcatgtctgccatggcccccacaccgccccagggtgaggt | |
| tgacgccgactgcatggacgtcaatgtccgcgggcctgatggcttcaccc | |
| cgctcatgatcgcctcctgcagcgggggcggcctggagacgggcaacagc | |
| gaggaagaggaggacgcgccggccgtcatctccgacttcatctaccaggg | |
| cgccagcctgcacaaccagacagaccgcacgggcgagaccgccttgcacc | |
| tggccgcccgctactcacgctctgatgccgccaagcgcctgctggaggcc | |
| agcgcagatgccaacatccaggacaacatgggccgcaccccgctgcatgc | |
| ggctgtgtctgccgacgcacaaggtgtcttccagatcctgatccggaacc | |
| gagccacagacctggatgcccgcatgcatgatggcacgacgccactgatc | |
| ctggctgcccgcctggccgtggagggcatgctggaggacctcatcaactc | |
| acacgccgacgtcaacgccgtagatgacctgggcaagtccgccctgcact | |
| gggccgccgccgtgaacaatgtggatgccgcagttgtgctcctgaagaac | |
| ggggctaacaaagatatgcagaacaacagggaggagacacccctgtttct | |
| ggccgcccgggagggcagctacgagaccgccaaggtgctgctggaccact | |
| ttgccaaccgggacatcacggatcatatggaccgcctgccgcgcgacatc | |
| gcacaggagcgcatgcatcacgacatcgtgaggctgctggacgagtacaa | |
| cctggtgcgcagcccgcagctgcacggagccccgctggggggcacgccca | |
| ccctgtcgcccccgctctgctcgcccaacggctacctgggcagcctcaag | |
| cccggcgtgcagggcaagaaggtccgcaagcccagcagcaaaggcctggc | |
| ctgtggaagcaaggaggccaaggacctcaaggcacggaggaagaagtccc | |
| aggacggcaagggctgcctgctggacagctccggcatgctctcgcccgtg | |
| gactccctggagtcaccccatggctacctgtcagacgtggcctcgccgcc | |
| actgctgccctccccgttccagcagtctccgtccgtgcccctcaaccacc | |
| tgcctgggatgcccgacacccacctgggcatcgggcacctgaacgtggcg | |
| gccaagcccgagatggcggcgctgggtgggggcggccggctggcctttga | |
| gactggcccacctcgtctctcccacctgcctgtggcctctggcaccagca | |
| ccgtcctgggctccagcagcggaggggccctgaatttcactgtgggcggg | |
| tccaccagtttgaatggtcaatgcgagtggctgtcccggctgcagagcgg | |
| catggtgccgaaccaatacaaccctctgcgggggagtgtggcaccaggcc | |
| ccctgagcacacaggccccctccctgcagcatggcatggtaggcccgctg | |
| cacagtagccttgctgccagcgccctgtcccagatgatgagctaccaggg | |
| cctgcccagcacccggctggccacccagcctcacctggtgcagacccagc | |
| aggtgcagccacaaaacttacagatgcagcagcagaacctgcagccagca | |
| aacatccagcagcagcaaagcctgcagccgccaccaccaccaccacagcc | |
| gcaccttggcgtgagctcagcagccagcggccacctgggccggagcttcc | |
| tgagtggagagccgagccaggcagacgtgcagccactgggccccagcagc | |
| ctggcggtgcacactattctgccccaggagagccccgccctgcccacgtc | |
| gctgccatcctcgctggtcccacccgtgaccgcagcccagttcctgacgc | |
| ccccctcgcagcacagctactcctcgcctgtggacaacacccccagccac | |
| cagctacaggtgcctgagcaccccttcctcaccccgtcccctgagtcccc | |
| tgaccagtggtccagctcgtccccgcattccaacgtctccgactggtccg | |
| agggcgtctccagccctcccaccagcatgcagtcccagatcgcccgcatt | |
| ccggaggccttcaagtaa | |
| FATI | SEQ ID |
| atggggagacatttggctttgctcctgcttctgctccttctcttccaaca | NO: 327 |
| ttttggagacagtgatggcagccaacgacttgaacagactcctctgcagt | |
| ttacacacctcgagtacaacgtcaccgtgcaggagaactctgcagctaag | |
| acttatgtggggcatcctgtcaagatgggtgtttacattacacatccagc | |
| gtgggaagtaaggtacaaaattgtttccggagacagtgaaaacctgttca | |
| aagctgaagagtacattctcggagacttttgctttctaagaataaggacc | |
| aaaggaggaaatacagctattcttaatagagaagtgaaggatcactacac | |
| attgatagtgaaagcacttgaaaaaaatactaatgtggaggcgcgaacaa | |
| aggtcagggtgcaggtgctggatacaaatgacttgagaccgttattctca | |
| cccacctcatacagcgtttctttacctgaaaacacagctataaggaccag | |
| tatcgcaagagtcagcgccacggatgcagacataggaaccaacggggaat | |
| tttactacagttttaaagatcgaacagatatgtttgctattcacccaacc | |
| agtggtgtgatagtgttaactggtagacttgattacctagagaccaagct | |
| ctatgagatggaaatcctcgctgcggaccgtggcatgaagttgtatggga | |
| gcagtggcatcagcagcatggccaagctaacggtgcacatcgaacaggcc | |
| aatgaatgtgctccggtgataacagcagtgacattgtcaccatcagaact | |
| ggacagggacccagcatatgcaattgtgacagtggatgactgcgatcagg | |
| gtgccaatggtgacatagcatctttaagcatcgtggcaggtgaccttctc | |
| cagcagtttagaacagtgaggtcctttccagggagtaaggagtataaagt | |
| caaagccatcggtggcattgattgggacagtcatcctttcggctacaatc | |
| tcacactacaggctaaagataaaggaactccgccccagttctcttctgtt | |
| aaagtcattcacgtgacttctccacagttcaaagccgggccagtcaagtt | |
| tgaaaaggatgtttacagagcagaaataagtgaatttgctcctcccaaca | |
| cacctgtggtcatggtaaaggccattcctgcttattcccatttgaggtat | |
| gtttttaaaagtacacctggaaaagctaaattcagtttaaattacaacac | |
| tggtctcatttctattttagaaccagttaaaagacagcaggcagcccatt | |
| ttgaacttgaagtaacaacaagtgacagaaaagcgtccaccaaggtcttg | |
| gtgaaagtcttaggtgcaaatagcaatccccctgaatttacccagacagc | |
| gtacaaagctgcttttgatgagaacgtgcccattggtactactgtcatga | |
| gcctgagtgccgtagaccctgatgagggtgagaacgggtacgtgacatac | |
| agtatcgcaaatttaaatcatgtgccgtttgcgattgaccatttcactgg | |
| tgccgtgagtacgtcagaaaacctggactacgaactgatgcctcgggttt | |
| atactctgaggattcgtgcatcagactggggcttgccgtaccgccgggaa | |
| gtcgaagtccttgctacaattactctcaataacttgaatgacaacacacc | |
| tttgtttgagaaaataaattgtgaagggacaattcccagagatctaggcg | |
| tgggagagcaaataaccactgtttctgctattgatgcagatgaacttcag | |
| ttggtacagtatcagattgaagctggaaatgaactggatttctttagttt | |
| aaaccccaactcgggggtattgtcattaaagcgatcgctaatggatggct | |
| taggtgcaaaggtgtctttccacagtctgagaatcacagctacagatgga | |
| gaaaattttgccacaccattatatatcaacataacagtggctgccagtca | |
| caagctggtaaacttgcagtgtgaagagactggtgttgccaaaatgctgg | |
| cagagaagctcctgcaggcaaataaattacacaaccagggagaggtggag | |
| gatattttcttcgattctcactctgtcaatgctcacataccgcagtttag | |
| aagcactcttccgactggtattcaggtaaaggaaaaccagcctgtgggtt | |
| ccagtgtaattttcatgaactccactgaccttgacactggcttcaatgga | |
| aaactggtctatgctgtttctggaggaaatgaggatagttgcttcatgat | |
| tgatatggaaacaggaatgctgaaaattttatctcctcttgaccgtgaaa | |
| caacagacaaatacaccctgaatattaccgtctatgaccttgggataccc | |
| cagaaggctgcgtggcgtcttctacatgtcgtggttgtcgatgccaatga | |
| taatccacccgagtttttacaggagagctattttgtggaagtgagtgaag | |
| acaaggaggtacatagtgaaatcatccaggttgaagccacagataaagac | |
| ctggggcccaacggacacgtgacgtactcaattgttacagacacagacac | |
| attttcaattgacagcgtgacgggtgttgttaacatcgcacgccctctgg | |
| atcgagagctgcagcatgagcactccttaaagattgaggccagggaccaa | |
| gccagagaagagcctcagctgttctccactgtcgttgtgaaagtatcact | |
| agaagatgttaatgacaacccacctacatttattccacctaattatcgtg | |
| tgaaagtccgagaggatcttccagaaggaaccgtcatcatgtggttagaa | |
| gcccacgatcctgatttaggtcagtctggtcaggtgagatacagccttct | |
| ggaccacggagaaggaaacttcgatgtggataaactcagtggagcagtta | |
| ggatcgtccagcagttggactttgagaagaagcaagtgtataatctcact | |
| gtgagggccaaagacaagggaaagccagtttctctgtcttctacttgcta | |
| tgttgaagttgaggtggttgatgtgaatgagaacctgcacccacccgtgt | |
| tttccagctttgtggaaaaggggacagtgaaagaagatgcacctgttggt | |
| tcattggtaatgacggtgtcggctcatgatgaggacgccagaagagatgg | |
| ggagatccgatactccattagagatggctctggcgttggtgttttcaaaa | |
| taggtgaagagacaggtgtcatagagacgtcagatcgactggaccgtgaa | |
| tcgacctcccattattggctaacagtctttgcaaccgatcagggtgtcgt | |
| gcctctttcatcgttcatagagatctacatagaggttgaggatgtcaatg | |
| acaatgcaccacagacatcagagcctgtttattacccagaaatcatggaa | |
| aattctcctaaagatgtatctgtggtccagatcgaggcatttgatccaga | |
| ttcgagctctaatgacaagctcatgtacaaaattacaagtggaaatccac | |
| aaggattcttttcaatacatcctaaaacaggtctcatcacaactacgtca | |
| aggaagctagaccgagaacagcaagatgaacacatattagaggttactgt | |
| gacagacaatggtagtccccccaaatcaaccattgcaagagtcattgtga | |
| aaatccttgatgaaaatgacaacaaacctcagtttctgcaaaagttctac | |
| aaaatcagactccctgagcgggaaaagccagaccgagaaagaaatgccag | |
| acgggagccgctctatcacgtcatagccaccgacaaggatgagggcccca | |
| atgcagaaatctcctacagcatcgaagacgggaatgagcatggcaaattt | |
| ttcatcgaaccgaaaactggagtggtttcgtccaagaggttttcagcagc | |
| tggagaatatgatattctttcaattaaggcagttgacaatggtcgccctc | |
| aaaagtcatcaaccaccagactccatattgaatggatctccaagcccaaa | |
| ccgtccctggagcccatttcatttgaagaatcattttttacctttactgt | |
| gatggaaagtgaccccgttgctcacatgattggagtaatatctgtggagc | |
| ctcctggcatacccctttggtttgacatcactggtggcaactacgacagt | |
| cacttcgatgtggacaagggaactggaaccatcattgttgccaaacctct | |
| tgatgcagaacagaagtcaaactacaacctcacagtcgaggctacagatg | |
| gaaccaccactatcctcactcaggtattcatcaaagtaatagacacaaat | |
| gaccatcgtcctcagttttctacatcaaagtatgaagttgttattcctga | |
| agatacagcgccagaaacagaaattttgcaaatcagtgctgtggatcagg | |
| atgagaaaaacaaactaatctacactctgcagagcagtagagatccactg | |
| agtctcaagaaatttcgtcttgatcctgcaaccggctctctctatacttc | |
| tgagaaactggatcatgaagctgttcaccagcacaccctcacggtcatgg | |
| tacgagatcaagatgtgcctgtaaaacgcaactttgcaaggattgtggtc | |
| aatgtcagcgacacgaatgaccacgccccgtggttcaccgcttcctccta | |
| caaagggcgggtttatgaatcggcagccgttggctcagttgtgttgcagg | |
| tgacggctctggacaaggacaaagggaaaaatgctgaagtgctgtactcg | |
| atcgagtcaggaaatattggaaattcttttatgattgatcctgtcttggg | |
| ctctattaaaactgccaaagaattagatcgaagtaaccaagcggagtatg | |
| atttaatggtaaaagctacagataagggcagtccaccaatgagtgaaata | |
| acttctgtgcgtatctttgtcacaattgctgacaacgcctctccgaagtt | |
| tacatcaaaagaatattctgttgaacttagtgaaactgtcagcattggga | |
| gtttcgttgggatggttacagcccatagtcaatcatcagtggtgtatgaa | |
| ataaaagatggaaatacaggtgatgcttttgatattaatccacattctgg | |
| aactatcatcactcagaaagccctggactttgaaactttgcccatttaca | |
| cattgataatacaaggaactaacatggctggtttgtccactaatacaacg | |
| gttctagttcacttgcaggatgagaatgacaacgcgccagtttttatgca | |
| ggcagaatatacaggactcattagtgaatcagcctcaattaacagcgtgg | |
| tcctaacagacaggaatgtcccactggtgattcgagcagctgatgctgat | |
| aaagactcaaatgctttgcttgtatatcacattgttgaaccatctgtaca | |
| cacatattttgctattgattctagcactggtgctattcatacagtactaa | |
| gtctggactatgaagaaacaagtatttttcactttaccgtccaagtgcat | |
| gacatgggaaccccacgtttatttgctgagtatgcagcgaatgtaacagt | |
| acatgtaattgacattaatgactgcccccctgtgtttgccaagccattat | |
| atgaagcatctcttttgttaccaacatacaaaggagtaaaagtcatcaca | |
| gtaaatgctacagatgctgattcaagtgcattctcacagttgatttactc | |
| catcaccgaaggcaacatcggggagaagttttctatggactacaagactg | |
| gtgctctcactgtccaaaacacaactcagttaagaagccgctacgagcta | |
| accgttagagcttccgatggcagatttgccggccttacctctgtcaaaat | |
| taatgtgaaagaaagcaaagaaagtcacctaaagtttacccaggatgtct | |
| actctgcggtagtgaaagagaattccaccgaggccgaaacattagctgtc | |
| attactgctattgggaatccaatcaatgagcctttgttttatcacatcct | |
| caacccagatcgcagatttaaaataagccgcacttcaggagttctgtcaa | |
| ccactggcacgcccttcgatcgtgagcagcaggaggcgtttgatgtggtt | |
| gtagaagtgacagaggaacataagccttctgcagtggcccacgttgtcgt | |
| gaaggtcattgtagaagaccaaaatgataatgcgccggtgtttgtcaacc | |
| ttccctactacgccgttgttaaagtggacactgaggtgggccatgtcatt | |
| cgctatgtcactgctgtagacagagacagtggcagaaacggggaagtgca | |
| ttactacctcaaggaacatcatgaacactttcaaattggacccttgggtg | |
| aaatttcactgaaaaagcaatttgagcttgacaccttaaataaagaatat | |
| cttgttacagtggttgcaaaagatggagggaacccggccttttcagcgga | |
| agttatcgttccgatcactgtcatgaataaagccatgcctgtgtttgaaa | |
| aacctttctacagtgcagagattgcagagagcatccaggtgcacagccct | |
| gtggtccacgtgcaggctaacagcccggaaggcctgaaagtgttctacag | |
| catcacagacggagaccctttcagccagttcactattaacttcaatactg | |
| gagttatcaatgtcatagctcctctggactttgaggcccacccggcatat | |
| aagctgagcatacgcgcaactgactccttgacgggcgctcatgctgaagt | |
| atttgtggacatcatagtagacgacatcaatgataaccctcctgtgtttg | |
| ctcagcagtcttatgcggtgaccctgtctgaggcatctgtaattggaacg | |
| tctgttgttcaagttagagccaccgattctgattcagaaccaaatagagg | |
| aatctcataccagatgtttgggaatcacagcaagagtcatgatcattttc | |
| atgtagacagcagcactggcctcatctcactactcagaaccctggattac | |
| gagcagtcccggcagcacacgatttttgtgagggcagttgatggtggtat | |
| gcccacgctgagcagtgatgtgattgtcacggtggacgttaccgacctca | |
| atgataatccaccactctttgaacaacagatttatgaagccagaattagc | |
| gagcacgcccctcatgggcatttcgtgacctgtgtaaaagcctatgatgc | |
| agacagttcagacatagacaagttgcagtattccattctgtctggcaatg | |
| atcataaacattttgtcattgacagtgcaacagggattatcaccctctca | |
| aacctgcaccggcacgccctgaagccattttacagtcttaacctgtcagt | |
| gtctgatggagtttttagaagttccacccaggttcatgtaactgtaattg | |
| gaggcaatttgcacagtcctgctttccttcagaacgaatatgaagtggaa | |
| ctagctgaaaacgctcccctacataccctggtgatggaggtgaaaactac | |
| ggatggggattctggtatttatggtcacgttacttaccatattgtaaatg | |
| actttgccaaagacagattttacataaatgagagaggacagatatttact | |
| ttggaaaaacttgatcgagaaaccccggcggagaaagtgatctcagtccg | |
| tttaatggctaaggatgctggaggaaaagttgctttctgcaccgtgaatg | |
| tcatccttacagatgacaatgacaatgcaccacaatttcgagcaaccaaa | |
| tacgaagtgaatatcgggtccagtgctgctaaagggacttcagtcgttaa | |
| agttcttgcaagtgatgccgatgagggctccaatgccgacatcacctatg | |
| ccattgaagcagactctgaaagtgtaaaagagaatttggaaattaacaaa | |
| ctgtccggcgtaatcactacaaaggagagcctcattggcttggaaaatga | |
| attcttcactttctttgttagagctgtggataatgggtctccatcaaaag | |
| aatctgttgttcttgtctatgttaaaatccttccaccggaaatgcagctt | |
| ccaaaattttcagaacctttctatacctttacagtgtcagaggacgtgcc | |
| tattggaacagagatagatctcatccgagcagaacatagtgggactgttc | |
| tttacagcctggtcaaagggaatactccagaaagcaatagggatgagtcc | |
| tttgtgattgacagacagagcgggagactgaagttggagaagagtcttga | |
| tcatgagacaactaagtggtatcagttttccatactggccaggtgcactc | |
| aagatgaccatgagatggtggcttctgtagatgttagtatccaagtgaaa | |
| gatgcaaatgacaacagcccggtctttgaatctagtccatatgaggcatt | |
| cattgttgaaaacctgccagggggaagtagagtaattcagatcagggcat | |
| ctgatgctgactcaggaaccaacggccaagttatgtatagcctggatcag | |
| tcacaaagtgtggaagtcattgaatcctttgccattaacatggaaacagg | |
| ctggattacaactttaaaggaacttgaccatgaaaagagagacaattacc | |
| agattaaagtggttgcatcagatcatggtgaaaagatccagctatcctcc | |
| acagccattgtggatgttaccgtcaccgatgtcaacgatagtccaccacg | |
| attcacggccgagatctataaagggactgtgagtgaggatgacccccaag | |
| gtggggtgattgccatcttaagtaccacggatgctgattctgaagagatc | |
| aacagacaagttacatatttcataacaggaggggatcctttaggacagtt | |
| tgccgttgaaactatacagaatgaatggaaggtatatgtgaagaaacctc | |
| tagacagggaaaaaagggacaattaccttcttactatcacggcaactgat | |
| ggcaccttctcatcaaaagcgatagttgaagtgaaagttctggatgcaaa | |
| tgacaacagtccagtttgtgaaaagactttatattcagacactattcctg | |
| aagacgtccttcctggaaaattgatcatgcagatctctgctacagacgca | |
| gacatccgctctaacgctgaaattacttacacgttattgggttcaggtgc | |
| agaaaaattcaaactaaatccagacacaggtgaactgaaaacgtcaaccc | |
| cccttgatcgtgaggagcaagctgtttatcatcttctcgtcagggccaca | |
| gatggaggaggaagattctgccaagccagtattgtgctcacgctagaaga | |
| tgtgaacgataacgcccccgaattctctgccgatccttatgccatcaccg | |
| tgtttgaaaacacagagccgggaacgctgctgacaagagtgcaggccaca | |
| gatgccgacgcaggattaaatcggaagattttatactcactgattgactc | |
| tgctgatgggcagttctccattaacgaattatctggaattattcagttag | |
| aaaaacctttggacagagaactccaggcagtatacaccctctctttgaaa | |
| gctgtggatcaaggcttgccaaggaggctgactgccactggcactgtgat | |
| tgtatcagttcttgacataaatgacaacccccctgtgtttgagtaccgtg | |
| aatatggtgccaccgtgtctgaggacattcttgttggaactgaagttctt | |
| caagtgtatgcagcaagtcgggatattgaagcaaatgcagaaatcaccta | |
| ctcaataataagtggaaatgaacatgggaaattcagcatagattctaaaa | |
| caggggccgtatttatcattgagaatctggattatgagagctctcatgag | |
| tattacctaacagtagaggccactgatggaggcacgccttcactgagcga | |
| cgttgccactgtgaacgttaatgtaacagatatcaacgataatacccctg | |
| tgttcagccaagacacctacacgacagtcatcagtgaagatgccgttctt | |
| gagcagtctgtcatcacggttatggccgatgatgccgatggaccttccaa | |
| cagccacatccactactcaattatagatggcaaccaaggaagctcgttca | |
| caattgaccccgtcaggggagaagtcaaagtgaccaaacttctcgaccga | |
| gaaacgatttcaggttacacgctcacggttcaagcttctgataatggcag | |
| tccacccagagtcaacacgacgaccgtgaacatcgatgtgtccgatgtca | |
| atgacaacgcgcccgtcttctccaggggaaactacagtgtcattatccag | |
| gaaaataagccagtgggcttcagcgtgctgcagctggtagtaacagatga | |
| ggattcttcccataacggtccacccttcttctttactattgtaactggaa | |
| atgatgagaaggcttttgaagttaacccgcaaggagtcctcctgacatca | |
| tctgccatcaagaggaaggagaaagatcattacttactgcaggtgaaggt | |
| ggcagataatggaaagcctcagttgtcatctttgacatacattgacatta | |
| gggtaattgaggagagcatctatccgcctgcgattttgcccctggagatt | |
| ttcatcacctcttctggagaagaatactcaggtggcgtcattgggaagat | |
| ccatgccacagaccaggacgtgtatgatactctaacctacagtctcgacc | |
| ctcagatggacaacctgttctctgtttccagcacagggggcaagctgata | |
| gcacacaaaaagctagacatagggcaataccttctcaatgtcagcgtaac | |
| agatgggaagttcacgacggtggccgacatcacagtgcatatcagacaag | |
| tcacacaggagatgttgaaccacaccatcgcgatccgctttgccaacctc | |
| actccggaagaattcgttggtgactactggcgcaacttccagcgagcttt | |
| acggaacatcctgggtgtgaggaggaacgacatacagattgttagtttgc | |
| agtcctctgaacctcacccacatctggacgtcttactttttgtagagaaa | |
| ccaggtagtgctcagatctcaacaaaacaacttctgcacaagattaactc | |
| ttccgtgactgacattgaggaaatcattggagttaggatactgaatgtat | |
| tccagaaactctgcgcgggactggactgcccctggaagttctgcgatgaa | |
| aaggtgtctgtggatgaaagtgtgatgtcaacacacagcacagccagact | |
| gagttttgtgactccccgccaccacagggcagcggtgtgtctctgcaaag | |
| agggaaggtgcccacctgtccaccatggctgtgaagatgatccgtgccct | |
| gagggatccgaatgtgtgtctgatccctgggaggagaaacacacctgtgt | |
| ctgtcccagcggcaggtttggtcagtgcccagggagttcatctatgacac | |
| tgactggaaacagctacgtgaaataccgtctgacggaaaatgaaaacaaa | |
| ttagagatgaaactgaccatgaggctcagaacatattccacgcatgcggt | |
| tgtcatgtatgctcgaggaactgactatagcatcttggagattcatcatg | |
| gaaggctgcagtacaagtttgactgtggaagtggccctggaattgtctct | |
| gttcagagcattcaggtcaatgatgggcagtggcacgcagtggccctgga | |
| agtgaatggaaactatgctcgcttggttctagaccaagttcatactgcat | |
| cgggcacagccccagggactctgaaaaccctgaacctggataactatgtg | |
| ttttttggtggccacatccgtcagcagggaacaaggcatggaagaagtcc | |
| tcaagttggtaatggtttcaggggttgtatggactccatttatttgaatg | |
| ggcaggagctccctttaaacagcaaacccagaagctatgcacacatcgaa | |
| gagtcggtggatgtatctccaggctgcttcctgacggccacggaagactg | |
| cgccagcaacccttgccagaatggaggcgtttgcaatccgtcacctgctg | |
| gaggttattactgcaaatgcagtgccttgtacatagggacccactgtgag | |
| ataagcgtcaatccgtgttcctccaagccatgcctctatgggggcacgtg | |
| tgttgtcgacaacggaggctttgtttgccagtgtagaggattatatactg | |
| gtcagaggtgtcagcttagtccatactgcaaagatgaaccctgtaagaat | |
| ggcggaacatgctttgacagtttggatggcgccgtttgtcagtgtgattc | |
| gggttttaggggagaaaggtgtcagagtgatatcgacgagtgctctggaa | |
| acccttgcctgcacggggccctctgtgagaacacgcacggctcctatcac | |
| tgcaactgcagccacgagtacaggggacgtcactgcgaggatgctgcgcc | |
| caaccagtatgtgtccacgccgtggaacattgggttggcggaaggaattg | |
| gaatcgttgtgtttgttgcagggatatttttactggtggtggtgtttgtt | |
| ctctgccgtaagatgattagtcggaaaaagaagcatcaggctgaacctaa | |
| agacaagcacctgggacccgctacggctttcttgcaaagaccgtattttg | |
| attccaagctaaataagaacatttactcagacataccaccccaggtgcct | |
| gtccggcctatttcctacaccccgagtattccaagtgactcaagaaacaa | |
| tctggaccgaaattccttcgaaggatctgctatcccagagcatcccgaat | |
| tcagcacttttaaccccgagtctgtgcacgggcaccgaaaagcagtggcg | |
| gtctgcagcgtggcgccaaacctgcctcccccacccccttcaaactcccc | |
| ttctgacagcgactccatccagaagcctagctgggactttgactatgaca | |
| caaaagtggtggatcttgatccctgtctttccaagaagcctctagaggaa | |
| aagccttcccagccatacagtgcccgggaaagcctgtctgaagtgcagtc | |
| tctgagctccttccagtccgaatcgtgcgatgacaatgggtatcactggg | |
| atacatcagattggatgccaagcgttcctctgccggacatacaagagttc | |
| cccaactatgaggtgattgatgagcagacacccctgtactcagcagatcc | |
| aaacgccatcgatacggactattaccctggaggctacgacatcgaaagtg | |
| attttcctccacccccagaagacttccccgcagctgatgagctaccaccg | |
| ttaccgcccgaattcagcaatcagtttgaatccatccaccctcctagaga | |
| catgcctgccgcgggtagcttgggttcttcatcaagaaaccggcagaggt | |
| tcaacttgaatcagtatttgcccaatttttatcccctcgatatgtctgaa | |
| cctcaaacaaaaggcactggtgagaatagtacttgtagagaaccccatgc | |
| cccttacccgccagggtatcaaagacacttcgaggcgcccgctgtcgaga | |
| gcatgcccatgtctgtgtacgcctccaccgcctcctgctctgacgtgtca | |
| gcctgctgcgaagtggagtccgaggtcatgatgagtgactatgagagcgg | |
| ggacgacggccacttcgaagaggtgacgatcccgcccctggattcccagc | |
| agcacacggaagtctga | |
| RBI | SEQ ID |
| atgccgcccaaaaccccccgaaaaacggccgccaccgccgccgctgccgc | NO: 328 |
| cgcggaacccccggcaccgccgccgccgccccctcctgaggaggacccag | |
| agcaggacagcggcccggaggacctgcctctcgtcaggcttgagtttgaa | |
| gaaacagaagaacctgattttactgcattatgtcagaaattaaagatacc | |
| agatcatgtcagagagagagcttggttaacttgggagaaagtttcatctg | |
| tggatggagtattgggaggttatattcaaaagaaaaaggaactgtgggga | |
| atctgtatctttattgcagcagttgacctagatgagatgtcgttcacttt | |
| tactgagctacagaaaaacatagaaatcagtgtccataaattctttaact | |
| tactaaaagaaattgataccagtaccaaagttgataatgctatgtcaaga | |
| ctgttgaagaagtatgatgtattgtttgcactcttcagcaaattggaaag | |
| gacatgtgaacttatatatttgacacaacccagcagttcgatatctactg | |
| aaataaattctgcattggtgctaaaagtttcttggatcacatttttatta | |
| gctaaaggggaagtattacaaatggaagatgatctggtgatttcatttca | |
| gttaatgctatgtgtccttgactattttattaaactctcacctcccatgt | |
| tgctcaaagaaccatataaaacagctgttatacccattaatggttcacct | |
| cgaacacccaggcgaggtcagaacaggagtgcacggatagcaaaacaact | |
| agaaaatgatacaagaattattgaagttctctgtaaagaacatgaatgta | |
| atatagatgaggtgaaaaatgtttatttcaaaaattttataccttttatg | |
| aattctcttggacttgtaacatctaatggacttccagaggttgaaaatct | |
| ttctaaacgatacgaagaaatttatcttaaaaataaagatctagatgcaa | |
| gattatttttggatcatgataaaactcttcagactgattctatagacagt | |
| tttgaaacacagagaacaccacgaaaaagtaaccttgatgaagaggtgaa | |
| tgtaattcctccacacactccagttaggactgttatgaacactatccaac | |
| aattaatgatgattttaaattcagcaagtgatcaaccttcagaaaatctg | |
| atttcctattttaacaactgcacagtgaatccaaaagaaagtatactgaa | |
| aagagtgaaggatataggatacatctttaaagagaaatttgctaaagctg | |
| tgggacagggttgtgtcgaaattggatcacagcgatacaaacttggagtt | |
| cgcttgtattaccgagtaatggaatccatgcttaaatcagaagaagaacg | |
| attatccattcaaaattttagcaaacttctgaatgacaacatttttcata | |
| tgtctttattggcgtgcgctcttgaggttgtaatggccacatatagcaga | |
| agtacatctcagaatcttgattctggaacagatttgtctttcccatggat | |
| tctgaatgtgcttaatttaaaagcctttgatttttacaaagtgatcgaaa | |
| gttttatcaaagcagaaggcaacttgacaagagaaatgataaaacattta | |
| gaacgatgtgaacatcgaatcatggaatcccttgcatggctctcagattc | |
| acctttatttgatcttattaaacaatcaaaggaccgagaaggaccaactg | |
| atcaccttgaatctgcttgtcctcttaatcttcctctccagaataatcac | |
| actgcagcagatatgtatctttctcctgtaagatctccaaagaaaaaagg | |
| ttcaactacgcgtgtaaattctactgcaaatgcagagacacaagcaacct | |
| cagccttccagacccagaagccattgaaatctacctctctttcactgttt | |
| tataaaaaagtgtatcggctagcctatctccggctaaatacactttgtga | |
| acgccttctgtctgagcacccagaattagaacatatcatctggacccttt | |
| tccagcacaccctgcagaatgagtatgaactcatgagagacaggcatttg | |
| gaccaaattatgatgtgttccatgtatggcatatgcaaagtgaagaatat | |
| agaccttaaattcaaaatcattgtaacagcatacaaggatcttcctcatg | |
| ctgttcaggagacattcaaacgtgttttgatcaaagaagaggagtatgat | |
| tctattatagtattctataactcggtcttcatgcagagactgaaaacaaa | |
| tattttgcagtatgcttccaccaggccccctaccttgtcaccaatacctc | |
| acattcctcgaagcccttacaagtttcctagttcacccttacggattcct | |
| ggagggaacatctatatttcacccctgaagagtccatataaaatttcaga | |
| aggtctgccaacaccaacaaaaatgactccaagatcaagaatcttagtat | |
| caattggtgaatcattcgggacttctgagaagttccagaaaataaatcag | |
| atggtatgtaacagcgaccgtgtgctcaaaagaagtgctgaaggaagcaa | |
| ccctcctaaaccactgaaaaaactacgctttgatattgaaggatcagatg | |
| aagcagatggaagtaaacatctcccaggagagtccaaatttcagcagaaa | |
| ctggcagaaatgacttctactcgaacacgaatgcaaaagcagaaaatgaa | |
| tgatagcatggatacctcaaacaaggaagagaaatga | |
| CASP8 | SEQ ID |
| atggagggaggcagaagagccagggtggttattgaaagtaaaagaaactt | NO: 329 |
| cttcctgggagcctttcccacccccttccctgctgagcacgtggagttag | |
| gcaggttaggggactcggagactgcgatggtgccaggaaagggtggagcg | |
| gattatattctcctgccttttaaaaagatggacttcagcagaaatcttta | |
| tgatattggggaacaactggacagtgaagatctggcctccctcaagttcc | |
| tgagcctggactacattccgcaaaggaagcaagaacccatcaaggatgcc | |
| ttgatgttattccagagactccaggaaaagagaatgttggaggaaagcaa | |
| tctgtccttcctgaaggagctgctcttccgaattaatagactggatttgc | |
| tgattacctacctaaacactagaaaggaggagatggaaagggaacttcag | |
| acaccaggcagggctcaaatttctgcctacagggtcatgctctatcagat | |
| ttcagaagaagtgagcagatcagaattgaggtcttttaagtttcttttgc | |
| aagaggaaatctccaaatgcaaactggatgatgacatgaacctgctggat | |
| attttcatagagatggagaagagggtcatcctgggagaaggaaagttgga | |
| catcctgaaaagagtctgtgcccaaatcaacaagagcctgctgaagataa | |
| tcaacgactatgaagaattcagcaaagagagaagcagcagccttgaagga | |
| agtcctgatgaattttcaaatggggaggagttgtgtggggtaatgacaat | |
| ctcggactctccaagagaacaggatagtgaatcacagactttggacaaag | |
| tttaccaaatgaaaagcaaacctcggggatactgtctgatcatcaacaat | |
| cacaattttgcaaaagcacgggagaaagtgcccaaacttcacagcattag | |
| ggacaggaatggaacacacttggatgcaggggctttgaccacgacctttg | |
| aagagcttcattttgagatcaagccccacgatgactgcacagtagagcaa | |
| atctatgagattttgaaaatctaccaactcatggaccacagtaacatgga | |
| ctgcttcatctgctgtatcctctcccatggagacaagggcatcatctatg | |
| gcactgatggacaggaggcccccatctatgagctgacatctcagttcact | |
| ggtttgaagtgcccttcccttgctggaaaacccaaagtgttttttattca | |
| ggcttgtcagggggataactaccagaaaggtatacctgttgagactgatt | |
| cagaggagcaaccctatttagaaatggatttatcatcacctcaaacgaga | |
| tatatcccggatgaggctgactttctgctggggatggccactgtgaataa | |
| ctgtgtttcctaccgaaaccctgcagagggaacctggtacatccagtcac | |
| tttgccagagcctgagagagcgatgtcctcgaggcgatgatattctcacc | |
| atcctgactgaagtgaactatgaagtaagcaacaaggatgacaagaaaaa | |
| catggggaaacagatgcctcagcctactttcacactaagaaaaaaacttg | |
| tctteecttctgattga | |
| PTEN | SEQ ID |
| atgacagccatcatcaaagagatcgttagcagaaacaaaaggagatatca | NO: 330 |
| agaggatggattcgacttagacttgacctatatttatccaaacattattg | |
| ctatgggatttcctgcagaaagacttgaaggcgtatacaggaacaatatt | |
| gatgatgtagtaaggtttttggattcaaagcataaaaaccattacaagat | |
| atacaatctttgtgctgaaagacattatgacaccgccaaatttaattgca | |
| gagttgcacaatatccttttgaagaccataacccaccacagctagaactt | |
| atcaaacccttttgtgaagatcttgaccaatggctaagtgaagatgacaa | |
| tcatgttgcagcaattcactgtaaagctggaaagggacgaactggtgtaa | |
| tgatatgtgcatatttattacatcggggcaaatttttaaaggcacaagag | |
| gccctagatttctatggggaagtaaggaccagagacaaaaagggagtaac | |
| tattcccagtcagaggcgctatgtgtattattatagctacctgttaaaga | |
| atcatctggattatagaccagtggcactgttgtttcacaagatgatgttt | |
| gaaactattccaatgttcagtggcggaacttgcaatcctcagtttgtggt | |
| ctgccagctaaaggtgaagatatattcctccaattcaggacccacacgac | |
| gggaagacaagttcatgtactttgagttccctcagccgttacctgtgtgt | |
| ggtgatatcaaagtagagttcttccacaaacagaacaagatgctaaaaaa | |
| ggacaaaatgtttcacttttgggtaaatacattcttcataccaggaccag | |
| aggaaacctcagaaaaagtagaaaatggaagtctatgtgatcaagaaatc | |
| gatagcatttgcagtatagagcgtgcagataatgacaaggaatatctagt | |
| acttactttaacaaaaaatgatcttgacaaagcaaataaagacaaagcca | |
| accgatacttttctccaaattttaaggtgaagctgtacttcacaaaaaca | |
| gtagaggagccgtcaaatccagaggctagcagttcaacttctgtaacacc | |
| agatgttagtgacaatgaacctgatcattatagatattctgacaccactg | |
| actctgatccagagaatgaaccttttgatgaagatcagcatacacaaatt | |
| acaaaagtctga | |
| EPHA2 | SEQ ID |
| atggagctccaggcagcccgcgcctgcttcgccctgctgtggggctgtgc | NO: 331 |
| gctggccgcggccgcggcggcgcagggcaaggaagtggtactgctggact | |
| ttgctgcagctggaggggagctcggctggctcacacacccgtatggcaaa | |
| gggtgggacctgatgcagaacatcatgaatgacatgccgatctacatgta | |
| ctccgtgtgcaacgtgatgtctggcgaccaggacaactggctccgcacca | |
| actgggtgtaccgaggagaggctgagcgtatcttcattgagctcaagttt | |
| actgtacgtgactgcaacagcttccctggtggcgccagctcctgcaagga | |
| gactttcaacctctactatgccgagtcggacctggactacggcaccaact | |
| tccagaagcgcctgttcaccaagattgacaccattgcgcccgatgagatc | |
| accgtcagcagcgacttcgaggcacgccacgtgaagctgaacgtggagga | |
| gcgctccgtggggccgctcacccgcaaaggcttctacctggccttccagg | |
| atatcggtgcctgtgtggcgctgctctccgtccgtgtctactacaagaag | |
| tgccccgagctgctgcagggcctggcccacttccctgagaccatcgccgg | |
| ctctgatgcaccttccctggccactgtggccggcacctgtgtggaccatg | |
| ccgtggtgccaccggggggtgaagagccccgtatgcactgtgcagtggat | |
| ggcgagtggctggtgcccattgggcagtgcctgtgccaggcaggctacga | |
| gaaggtggaggatgcctgccaggcctgctcgcctggattttttaagtttg | |
| aggcatctgagagcccctgcttggagtgccctgagcacacgctgccatcc | |
| cctgagggtgccacctcctgcgagtgtgaggaaggcttcttccgggcacc | |
| tcaggacccagcgtcgatgccttgcacacgacccccctccgccccacact | |
| acctcacagccgtgggcatgggtgccaaggtggagctgcgctggacgccc | |
| cctcaggacagcgggggccgcgaggacattgtctacagcgtcacctgcga | |
| acagtgctggcccgagtctggggaatgcgggccgtgtgaggccagtgtgc | |
| gctactcggagcctcctcacggactgacccgcaccagtgtgacagtgagc | |
| gacctggagccccacatgaactacaccttcaccgtggaggcccgcaatgg | |
| cgtctcaggcctggtaaccagccgcagcttccgtactgccagtgtcagca | |
| tcaaccagacagagccccccaaggtgaggctggagggccgcagcaccacc | |
| tcgcttagcgtctcctggagcatccccccgccgcagcagagccgagtgtg | |
| gaagtacgaggtcacttaccgcaagaagggagactccaacagctacaatg | |
| tgcgccgcaccgagggtttctccgtgaccctggacgacctggccccagac | |
| accacctacctggtccaggtgcaggcactgacgcaggagggccagggggc | |
| cggcagcaaggtgcacgaattccagacgctgtccccggagggatctggca | |
| acttggcggtgattggcggcgtggctgtcggtgtggtcctgcttctggtg | |
| ctggcaggagttggcttctttatccaccgcaggaggaagaaccagcgtgc | |
| ccgccagtccccggaggacgtttacttctccaagtcagaacaactgaagc | |
| ccctgaagacatacgtggacccccacacatatgaggaccccaaccaggct | |
| gtgttgaagttcactaccgagatccatccatcctgtgtcactcggcagaa | |
| ggtgatcggagcaggagagtttggggaggtgtacaagggcatgctgaaga | |
| catcctcggggaagaaggaggtgccggtggccatcaagacgctgaaagcc | |
| ggctacacagagaagcagcgagtggacttcctcggcgaggccggcatcat | |
| gggccagttcagccaccacaacatcatccgcctagagggcgtcatctcca | |
| aatacaagcccatgatgatcatcactgagtacatggagaatggggccctg | |
| gacaagttccttcgggagaaggatggcgagttcagcgtgctgcagctggt | |
| gggcatgctgcggggcatcgcagctggcatgaagtacctggccaacatga | |
| actatgtgcaccgtgacctggctgcccgcaacatcctcgtcaacagcaac | |
| ctggtctgcaaggtgtctgactttggcctgtcccgcgtgctggaggacga | |
| ccccgaggccacctacaccaccagtggcggcaagatccccatccgctgga | |
| ccgccccggaggccatttcctaccggaagttcacctctgccagcgacgtg | |
| tggagctttggcattgtcatgtgggaggtgatgacctatggcgagcggcc | |
| ctactgggagttgtccaaccacgaggtgatgaaagccatcaatgatggct | |
| tccggctccccacacccatggactgcccctccgccatctaccagctcatg | |
| atgcagtgctggcagcaggagcgtgcccgccgccccaagttcgctgacat | |
| cgtcagcatcctggacaagctcattcgtgcccctgactccctcaagaccc | |
| tggctgactttgacccccgcgtgtctatccggctccccagcacgagcggc | |
| tcggagggggtgcccttccgcacggtgtccgagtggctggagtccatcaa | |
| gatgcagcagtatacggagcacttcatggcggccggctacactgccatcg | |
| agaaggtggtgcagatgaccaacgacgacatcaagaggattggggtgcgg | |
| ctgcccggccaccagaagcgcatcgcctacagcctgctgggactcaagga | |
| ccaggtgaacactgtggggatccccatctga | |
| TGFBR2 | SEQ ID |
| atgggtcgggggctgctcaggggcctgtggccgctgcacatcgtcctgtg | NO: 332 |
| gacgcgtatcgccagcacgatcccaccgcacgttcagaagtcggttaata | |
| acgacatgatagtcactgacaacaacggtgcagtcaagtttccacaactg | |
| tgtaaattttgtgatgtgagattttccacctgtgacaaccagaaatcctg | |
| catgagcaactgcagcatcacctccatctgtgagaagccacaggaagtct | |
| gtgtggctgtatggagaaagaatgacgagaacataacactagagacagtt | |
| tgccatgaccccaagctcccctaccatgactttattctggaagatgctgc | |
| ttctccaaagtgcattatgaaggaaaaaaaaaagcctggtgagactttct | |
| tcatgtgttcctgtagctctgatgagtgcaatgacaacatcatcttctca | |
| gaagaatataacaccagcaatcctgacttgttgctagtcatatttcaagt | |
| gacaggcatcagcctcctgccaccactgggagttgccatatctgtcatca | |
| tcatcttctactgctaccgcgttaaccggcagcagaagctgagttcaacc | |
| tgggaaaccggcaagacgcggaagctcatggagttcagcgagcactgtgc | |
| catcatcctggaagatgaccgctctgacatcagctccacgtgtgccaaca | |
| acatcaaccacaacacagagctgctgcccattgagctggacaccctggtg | |
| gggaaaggtcgctttgctgaggtctataaggccaagctgaagcagaacac | |
| ttcagagcagtttgagacagtggcagtcaagatctttccctatgaggagt | |
| atgcctcttggaagacagagaaggacatcttctcagacatcaatctgaag | |
| catgagaacatactccagttcctgacggctgaggagcggaagacggagtt | |
| ggggaaacaatactggctgatcaccgccttccacgccaagggcaacctac | |
| aggagtacctgacgcggcatgtcatcagctgggaggacctgcgcaagctg | |
| ggcagctccctcgcccgggggattgctcacctccacagtgatcacactcc | |
| atgtgggaggcccaagatgcccatcgtgcacagggacctcaagagctcca | |
| atatcctcgtgaagaacgacctaacctgctgcctgtgtgactttgggctt | |
| tccctgcgtctggaccctactctgtctgtggatgacctggctaacagtgg | |
| gcaggtgggaactgcaagatacatggctccagaagtcctagaatccagga | |
| tgaatttggagaatgttgagtccttcaagcagaccgatgtctactccatg | |
| gctctggtgctctgggaaatgacatctcgctgtaatgcagtgggagaagt | |
| aaaagattatgagcctccatttggttccaaggtgcgggagcacccctgtg | |
| tcgaaagcatgaaggacaacgtgttgagagatcgagggcgaccagaaatt | |
| cccagcttctggctcaaccaccagggcatccagatggtgtgtgagacgtt | |
| gactgagtgctgggaccacgacccagaggcccgtctcacagcccagtgtg | |
| tggcagaacgcttcagtgagctggagcatctggacaggctctcggggagg | |
| agctgctcggaggagaagattcctgaagacggctccctaaacactaccaa | |
| atag | |
| SDI | SEQ ID |
| atggatcagacctgtgaactacccagaagaaattgtctgctgcccttttc | NO: 333 |
| caatccagtgaatttagatgcccctgaagacaaggacagccctttcggta | |
| atggtcaatccaatttttctgagccacttaatgggtgtactatgcagtta | |
| tcgactgtcagtggaacatcccaaaatgcttatggacaagattctccatc | |
| ttgttacattccactgcggagactacaggatttggcctccatgatcaatg | |
| tagagtatttaaatgggtctgctgatggatcagaatcctttcaagaccct | |
| gaaaaaagtgattcaagagctcagacgccaattgtttgcacttccttgag | |
| tcctggtggtcctacagcacttgctatgaaacaggaaccctcttgtaata | |
| actcccctgaactccaggtaaaagtaacaaagactatcaagaatggcttt | |
| ctgcactttgagaattttacttgtgtggacgatgcagatgtagattctga | |
| aatggacccagaacagccagtcacagaggatgagagtatagaggagatct | |
| ttgaggaaactcagaccaatgccacctgcaattatgagactaaatcagag | |
| aatggtgtaaaagtggccatgggaagtgaacaagacagcacaccagagag | |
| tagacacggtgcagtcaaatcgccattcttgccattagctcctcagactg | |
| aaacacagaaaaataagcaaagaaatgaagtggacggcagcaatgaaaaa | |
| gcagcccttctcccagcccccttttcactaggagacacaaacattacaat | |
| agaagagcaattaaactcaataaatttatcttttcaggatgatccagatt | |
| ccagtaccagtacattaggaaacatgctagaattacctggaacttcatca | |
| tcatctacttcacaggaattgccattttgtcaacctaagaaaaagtctac | |
| gccactgaagtatgaagttggagatctcatctgggcaaaattcaagagac | |
| gcccatggtggccctgcaggatttgttctgatccgttgattaacacacat | |
| tcaaaaatgaaagtttccaaccggaggccctatcggcagtactacgtgga | |
| ggcttttggagatccttctgagagagcctgggtggctggaaaagcaatcg | |
| tcatgtttgaaggcagacatcaattcgaagagctacctgtccttaggaga | |
| agagggaaacagaaagaaaaaggatataggcataaggttcctcagaaaat | |
| tttgagtaaatgggaagccagtgttggacttgcagaacagtatgatgttc | |
| ccaaggggtcaaagaaccgaaaatgtattcctggttcaatcaagttggac | |
| agtgaagaagatatgccatttgaagactgcacaaatgatcctgagtcaga | |
| acatgacctgttgcttaatggctgtttgaaatcactggcttttgattctg | |
| aacattctgcagatgagaaggaaaagccttgcgctaaatctcgagccaga | |
| aagagctctgataatccaaaaaggactagtgtgaaaaagggccacataca | |
| atttgaagcacataaagatgaacggaggggaaagattccagagaaccttg | |
| gcctaaactttatctctggggatatatctgatacgcaggcctctaatgaa | |
| ctttccaggatagcaaatagcctcacagggtccaacactgccccaggaag | |
| ttttctgttttcttcctgtggaaaaaacactgcaaagaaagaatttgaga | |
| cttcaaatggtgactctttattgggcttgcctgagggtgctttgatctca | |
| aagtgttctcgagagaagaataaaccccaacgaagcctggtgtgtggttc | |
| aaaagtgaagctctgctatattggagcaggtgatgaggaaaagcgaagtg | |
| attccattagtatctgtaccacttctgatgatggaagcagtgacctggat | |
| cccatagaacacagctcagagtctgataacagtgtccttgaaattccaga | |
| tgctttcgatagaacagagaacatgttatctatgcagaaaaatgaaaaga | |
| taaagtattctaggtttgctgccacaaacactagggtaaaagcaaaacag | |
| aagcctctcattagtaactcacatacagaccacttaatgggttgtactaa | |
| gagtgcagagcctggaaccgagacgtctcaggttaatctctctgatctga | |
| aggcatctactcttgttcacaaaccccagtcagattttacaaatgatgct | |
| ctctctccaaaattcaacctgtcatcaagcatatccagtgagaactcgtt | |
| aataaagggtggggcagcaaatcaagctctattacattcgaaaagcaaac | |
| agcccaagttccgaagtataaagtgcaaacacaaagaaaatccagttatg | |
| gcagaacccccagttataaatgaggagtgcagtttgaaatgctgctcttc | |
| tgataccaaaggctctcctttggccagcatttctaaaagtgggaaagtgg | |
| atggtctaaaactactgaacaatatgcatgagaaaaccagggattcaagt | |
| gacatagaaacagcagtggtgaaacatgttttatccgagttgaaggaact | |
| ctcttacagatccttaggtgaggatgtcagtgactctggaacatcaaagc | |
| catcaaaaccattacttttctcttctgcttctagtcagaatcacatacct | |
| attgaaccagactacaaattcagtacattgctaatgatgttgaaagatat | |
| gcatgatagtaagacgaaggagcagcggttgatgactgctcaaaacctgg | |
| tctcttaccggagtcctggtcgtggggactgttctactaatagtcctgta | |
| ggagtctctaaggttttggtttcaggaggctccacacacaattcagagaa | |
| aaagggagatggcactcagaactccgccaatcctagccctagtgggggtg | |
| actctgcattatctggcgagttgtctgcttccctacctggcttactgtcc | |
| gacaagagagacctccctgcttctggtaaaagtcgttcagactgtgttac | |
| taggcgcaactgtggacgatcaaagccttcatccaaattgcgagatgctt | |
| tttcagcccaaatggtaaagaacacagtgaaccgtaaagccttaaagacc | |
| gagcgcaaaagaaaactgaatcagcttccaagtgtgactcttgatgctgt | |
| actgcagggagaccgagaacgtggaggttcattgagaggtggggcagaag | |
| atcctagtaaagaggatccccttcagataatgggccacttaacaagtgaa | |
| gatggtgaccatttttctgatgtgcatttcgatagcaaggttaagcaatc | |
| tgatcctggtaaaatttctgaaaaaggactctcttttgaaaacggaaaag | |
| gcccagagctggactctgtaatgaacagtgagaatgatgaactcaatggt | |
| gtaaatcaagtggtgcctaaaaagcggtggcagcgtttaaaccaaaggcg | |
| cactaaacctcgtaagcgcatgaacagatttaaagagaaagaaaactctg | |
| agtgtgcctttagggtcttacttcctagtgaccctgtgcaggaggggcgg | |
| gatgagtttccagagcatagaactccttcagcaagcatacttgaggaacc | |
| actgacagagcaaaatcatgctgactgcttagattcagctgggccacggt | |
| taaatgtttgtgataaatccagtgccagcattggtgacatggaaaaggag | |
| ccaggaattcccagtttgacaccacaggctgagctccctgaaccagctgt | |
| gcggtcagagaagaaacgccttaggaagccaagcaagtggcttttggaat | |
| atacagaagaatatgatcagatatttgctcctaagaaaaaacaaaagaag | |
| gtacaggagcaggtgcacaaggtaagttcccgctgtgaagaggaaagcct | |
| tctagcccgaggtcgatctagtgctcagaacaagcaggtggacgagaatt | |
| ctttgatttcaaccaaagaagagcctccagttcttgaaagggaggctccg | |
| tttttggagggccccttggctcagtcagaacttggaggtggacatgctga | |
| gttgccgcagctgaccttgtctgtgcctgtggctccggaagtctctccac | |
| ggcctgcccttgagtctgaggaattgctagttaaaacgccaggaaattat | |
| gaaagtaaacgtcaaagaaaaccaactaagaaacttcttgaatccaatga | |
| tttagaccctggatttatgcccaagaagggggaccttggcctttctaaaa | |
| agtgctatgaagctggtcacctggagaatggcataactgaatcttgtgcc | |
| acatcttattcaaaagattttggtggaggcactaccaagatatttgacaa | |
| gccaaggaagcgaaaacgacagaggcatgctgcagccaagatgcagtgta | |
| aaaaagtgaaaaatgatgactcgtcaaaagagattccaggctcagaggga | |
| gaactaatgcctcacaggacggccacaagccccaaggagactgttgagga | |
| aggtgtagaacacgatcccgggatgcctgcctctaaaaaaatgcagggtg | |
| aacgcggtggaggagctgcactcaaggagaatgtctgtcagaattgtgaa | |
| aaattgggtgagctgctgttatgtgaggctcagtgctgtggggctttcca | |
| cctggagtgccttggattgactgagatgccaagaggaaaatttatctgca | |
| atgaatgtcgcacaggaatccatacctgttttgtatgtaagcagagtggg | |
| gaagatgttaaaaggtgccttctacccttgtgtggaaagttttaccatga | |
| agagtgtgtccagaagtacccacccactgttatgcagaacaagggcttcc | |
| ggtgctccctccacatctgtataacctgtcatgctgctaatccagccaat | |
| gtttctgcatctaaaggtcggttgatgcgctgtgtccgctgtcctgtggc | |
| ataccacgccaatgacttttgcctggctgctgggtcaaagatccttgcat | |
| ctaatagtatcatctgccctaatcactttacccctaggcggggctgccga | |
| aatcatgagcatgttaatgttagctggtgctttgtgtgctcagaaggagg | |
| cagccttctgtgctgtgattcttgccctgctgcttttcatcgtgaatgcc | |
| tgaacattgatatccctgaaggaaactggtattgcaatgactgtaaagca | |
| ggcaaaaagccacactacagggagattgtctgggtaaaagttggacgata | |
| caggtggtggccagctgagatctgccatcctcgagctgttccttccaaca | |
| ttgataagatgagacatgatgtgggagagttcccagtcctcttttttgga | |
| tctaatgactatttgtggactcaccaggcccgagtcttcccttacatgga | |
| gggtgacgtgagcagcaaggataagatgggcaaaggagtggatgggacat | |
| ataaaaaagctcttcaggaagctgcagcaaggtttgaggaattaaaggcc | |
| caaaaagagctaagacagctgcaggaagaccgaaagaatgacaagaagcc | |
| accaccttataaacatataaaggtaaaccgtcctattggcagggtacaga | |
| tcttcactgcagacttatctgaaataccccgttgcaactgtaaagctact | |
| gatgagaacccctgtgggatagactctgaatgcatcaaccgcatgctgct | |
| ctatgagtgccaccccacagtgtgtcctgccggagggcgctgtcaaaacc | |
| agtgcttttccaagcgccaatatccagaggttgaaattttccgcacatta | |
| cagcggggttggggtctacggacaaaaacagatattaaaaagggtgaatt | |
| tgtgaatgagtatgtgggtgagcttatagatgaagaagaatgcagagctc | |
| gaattcgctatgctcaagaacatgatatcactaatttctatatgctcacc | |
| ctagacaaagaccgaatcattgatgctggtcccaaaggaaactatgctcg | |
| gttcatgaatcattgctgccagcccaactgtgaaacacagaagtggtctg | |
| tgaatggagatacccgtgtaggcctttttgcactaagtgacattaaagca | |
| ggcactgaacttaccttcaactacaacctagaatgtcttgggaatggaaa | |
| gactgtttgcaaatgtggagccccgaactgcagtggcttcttgggtgtaa | |
| ggccaaagaatcaacccattgccacggaagaaaagtcaaagaaattcaag | |
| aagaagcaacagggaaagcgcaggacccagggtgaaatcacaaaggagcg | |
| agaagatgagtgttttagttgtggggatgctggccagctcgtctcctgca | |
| agaaaccaggctgcccaaaagtttaccacgcagactgtctcaatctgacc | |
| aagcgaccagcagggaaatgggaatgtccgtggcatcagtgtgacatctg | |
| cgggaaggaagcagcctccttctgtgagatgtgccccagctccttttgta | |
| agcagcatcgagaagggatgcttttcatttccaaactggatgggcgtctg | |
| tcttgtactgagcatgacccctgtgggcccaatcctctggaacctgggga | |
| gatccgtgagtatgtgcctcccccagtaccgctgcctccagggccaagca | |
| ctcacctggcagagcaatcaacaggaatggctgctcaggcacccaaaatg | |
| tcagataaacctcctgctgacaccaaccagatgctgtcgctctccaaaaa | |
| agctctggcagggacttgtcagaggccattgctacctgaaagacctcttg | |
| agagaactgactccaggccccagcctttagataaggtcagagacctcgct | |
| gggtcagggaccaaatcccaatccttggtttccagccagaggccactgga | |
| caggccaccagcagtggcaggaccaagaccccagctaagcgacaaaccct | |
| ctccagtgaccagcccaagctcctcaccctcagtcaggtcccaaccactg | |
| gaaagacctctggggacggctgacccaaggctggataaatccataggtgc | |
| tgccagcccaaggccccagtcactggagaaaacctcagttcccactggcc | |
| tgagacttccgccgccagacagactgctcattactagcagtcccaaaccc | |
| cagacttcagacaggcctactgacaaaccccatgcctctttgtcccagag | |
| actcccacctcctgagaaagtactatcagctgtggtccagacccttgtag | |
| ctaaagaaaaagcactgaggcctgtggaccagaatactcagtcaaaaaat | |
| agagctgctttggtgatggatctcatagacctaactcctcgccagaagga | |
| gcgggcagcttcacctcatcaggtcacaccacaggctgatgagaagatgc | |
| cagtgttggagtcaagttcatggcctgccagcaaaggtctggggcatatg | |
| ccgagagctgttgagaaaggctgtgtgtcagatcctcttcagacatctgg | |
| gaaagcagcagccccttcagaggacccctggcaagctgttaaatcactca | |
| cccaggccagacttctttctcagcctcctgccaaggcctttttatatgag | |
| ccaacaactcaggcctcaggaagagcttctgcaggggctgagcagacccc | |
| agggcctcttagccaatccccgggcctggtgaagcaggcgaagcagatgg | |
| tcggaggccagcaactacctgcacttgccgccaagagtgggcaatctttt | |
| aggtctctcgggaaggccccagcctccctccccactgaagaaaagaagtt | |
| ggtaaccacagagcaaagtccctgggccctgggaaaagcctcatcacggg | |
| cagggctctggcccatagtggctggacagacactggcacagtcttgctgg | |
| tctgctgggagcacacagacattggcacagacttgctggtctcttggaag | |
| agggcaagaccccaaaccagagcaaaatacacttccagctcttaaccagg | |
| ctccttccagtcacaagtgtgcagaatcagaacagaagtag | |
| KMT2D | SEQ ID |
| atggacagccagaagctggctggtgaggataaagattcagaaccggcagc | NO: 334 |
| tgatggacctgcagcttctgaggacccaagtgccactgagtcagacctgc | |
| ccaacccacatgtgggagaggtctctgtccttagttctgggagtcccagg | |
| cttcaggagactcctcaggactgcagtgggggtccggtgcggcgttgtgc | |
| tctctgtaactgcggggagcccagtctacacgggcagcgggagctacggc | |
| gctttgagttgccatttgattggccccggtgtccagtggtgtcccctggg | |
| gggagcccagggcccaatgaggcagtgctgcccagtgaggacctatcaca | |
| gattggtttccctgagggccttacacctgcccacctaggagaacctggag | |
| ggtcctgctgggctcaccattggtgtgctgcatggtcggcaggcgtatgg | |
| gggcaggagggcccagaactatgtggtgtggacaaggccatcttctcagg | |
| gatctcacagcgctgctcccactgcaccaggctcggtgcctccatccctt | |
| gccgctcacctggatgtccacggctttaccacttcccctgcgcgactgcc | |
| agcggttccttcctatccatgaaaacactgcagctgctatgcccagagca | |
| cagtgagggggctgcatatctggaggaggctcgctgtgcagtgtgtgagg | |
| ggccaggggagttgtgtgacctgttcttctgtaccagctgtgggcatcac | |
| tatcacggggcctgcctggacactgctctgactgcccgcaaacgtgctgg | |
| ctggcagtgccctgaatgcaaagtgtgccaagcctgcaggaaacctggga | |
| atgactctaagatgttggtttgtgagacgtgtgacaaaggataccatact | |
| ttctgcctaaaaccacccatggaggaactgcctgctcactcttggaagtg | |
| caaggcgtgccgggtgtgccgggcctgtggggcgggctcagcagaactga | |
| atcccaactcggagtggtttgagaactactctctctgtcaccgctgtcac | |
| aaagcccagggaggtcagactatccgctccgttgctgagcagcatacccc | |
| ggtgtgtagcagattttcacccccagagcctggcgatacccccactgacg | |
| agcccgatgctctgtacgttgcatgccaagggcagccaaagggtgggcac | |
| gtgacctctatgcaacccaaggaaccagggcccctgcaatgtgaagccaa | |
| accactagggaaagcaggggtccaacttgagccccagttggaggcccccc | |
| taaacgaggagatgccactgctgcccccacctgaggagtcacccctgtcc | |
| ccaccacctgaggaatcacccacgtccccaccacctgaggcatcacgcct | |
| gtcaccaccacctgaggaattgcccgcatccccacttcctgaggcattgc | |
| acctgtcccggccgctggaggaatcgcccctctctccgccgcctgaggag | |
| tctcctctgtctcccccacctgaatcatcacctttttctccactggagga | |
| gtcgcccttgtctccaccggaagagtcacccccatctcctgcacttgaga | |
| cgcctctatccccaccacctgaagcatcgcccctgtccccaccatttgaa | |
| gaatctcctttgtccccgccacctgaggaattgcccacttccccgccacc | |
| tgaagcatctcgcctgtctccaccacctgaggagtcacccatgtcccctc | |
| cacctgaagagtcacccatgtctccaccaccggaggcatctcgtctgttc | |
| ccaccatttgaagagtctcctctgtcccctccacctgaggagtctcccct | |
| ttccccaccacctgaggcatcacgcctgtccccaccacctgaggactcgc | |
| ctatgtccccaccacctgaagaatcacctatgtcccccccacctgaggta | |
| tcgcgcctatcccccctgcctgtggtgtcacgcctgtctccaccgcctga | |
| ggaatctcccttgtccccaccgcctgaggagtctcccacgtcccctccac | |
| ctgaggcttcacgcctctccccaccacctgaggactcccccacatcccca | |
| ccacctgaggactcacctgcttccccaccaccggaggactcgctcatgtc | |
| cctgccgctggaggagtcacccctgttgccactacctgaggagccgcaac | |
| tctgcccccggtccgaggggccgcacctgtcaccccggcctgaggagccg | |
| cacctgtccccccggcctgaggagccacacctatctccgcaggctgagga | |
| gccacacctgtccccccagcctgaggagccatgcctatgcgctgtgcctg | |
| aggagccacacttgtccccccaggctgagggaccacatctgtcccctcag | |
| cctgaggaattgcacctgtccccccagactgaggagccgcacctgtctcc | |
| tgtgcctgaggagccatgcttgtccccccaacctgaggaatcacacctgt | |
| ccccccagtctgaggagccatgcctgtccccccggcctgaggaatcgcat | |
| ctgtcccctgagcttgagaagccacccctgtcccctcggcctgaaaagcc | |
| ccctgaggagccaggccaatgccctgcacctgaggagctgcccttgttcc | |
| ctccccctggggaaccatccttatctcccttgcttggagagccagccctg | |
| tctgagcctggggaaccacctctgtcccctctgcccgaggagctgccgtt | |
| gtccccatctggggagccatccttgtcgcctcagctgatgccaccagatc | |
| cccttcctcctccactctcacccatcatcacagctgcggccccaccggcc | |
| ctgtctcctttgggggagttagagtacccctttggtgccaaaggggacag | |
| tgaccctgagtcaccgttggctgcccccatcctggagacacccatcagcc | |
| ctccaccagaagctaactgcactgaccctgagcctgtcccccctatgatc | |
| cttcccccatctccaggctccccagtggggccggcttctcccatcctgat | |
| ggagccccttcctcctcagtgttcgccactccttcagcattccctggttc | |
| cccaaaactcccctccttcccagtgctctcctcctgccctaccactgtcc | |
| gttccctccccgttgagtcccatagggaaggtagtgggggtctcagatga | |
| ggctgagctgcacgagatggagactgagaaagtttcagaacctgaatgcc | |
| cagccttggaacccagtgccaccagtcctctcccttccccaatgggggac | |
| ctttcctgccccgcccccagccctgccccagccctggatgacttctctgg | |
| cctaggggaagacacagcccctctggatgggattgatgctccgggttcac | |
| agccagagcctggacagacccctggcagtttggctagtgaacttaaaggc | |
| tcccctgtgctcctggaccccgaggagctggcccctgtgacccctatgga | |
| ggtctaccccgaatgcaagcagacagcagggcagggctcaccatgtgaag | |
| aacaggaagagccacgtgcaccggtggcccccacaccacccactctcatc | |
| aaatccgacatcgttaacgagatctctaatctgagccagggtgatgccag | |
| tgccagttttcctggctcagagcccctcctgggctctccagacccggagg | |
| ggggtggctccctgtccatggagttgggggtctctacggatgttagtcca | |
| gcccgagatgagggctccctacggctctgtactgactcactgccagagac | |
| tgatgactcactattgtgcgatgctgggacagctatcagcggaggcaaag | |
| ctgagggggagaaggggcggcggcgcagctccccagcccgttcccgcatc | |
| aaacagggtcgcagcagcagtttcccaggaagacgccggcctcgtggagg | |
| agcccatggaggacgtggtagaggacgggcccggctaaagtcaactgctt | |
| cttccattgagactctggtagttgctgacattgatagctctcccagtaag | |
| gaggaggaggaagaagatgatgacaccatgcagaataccgtggttctctt | |
| ctccaacacagacaaatttgtcctaatgcaggacatgtgtgtggtatgtg | |
| gcagctttggccggggggcagagggccacctccttgcctgttcgcagtgc | |
| tctcagtgctatcacccttactgtgtcaacagcaagatcaccaaggtgat | |
| gctgctcaagggctggcgttgtgtggagtgtattgtgtgtgaggtgtgtg | |
| gccaggcctccgacccctcacgcctgctgctctgtgatgactgtgatatt | |
| agctaccacacatactgcctggaccccccactgctcaccgtccccaaggg | |
| cggctggaagtgcaagtggtgtgtgtcctgtatgcagtgtggggctgctt | |
| cccctggcttccactgtgaatggcagaatagttacacacactgtgggccc | |
| tgtgccagcctggtgacctgccctatctgtcatgctccttacgtagaaga | |
| ggacctactaatccagtgccgccactgtgaacggtggatgcatgcaggct | |
| gtgagagcctcttcacagaggacgatgtggagcaggcagccgatgaaggc | |
| tttgactgtgtctcctgccagccctacgtggtaaagcctgtggcgcctgt | |
| tgcacctccagagctggtgcccatgaaggtgaaagagccagagccccagt | |
| actttcgcttcgaaggtgtgtggctgacagaaactggcatggccttgctg | |
| cgtaacctgaccatgtcaccactgcacaagcggcgccaacggcgaggacg | |
| gcttggcctcccaggcgaggcaggattggagggttctgagccctcagatg | |
| cccttggccctgatgacaagaaggatggggacctggacaccgatgagctg | |
| ctcaagggtgaaggtggtgtggagcacatggagtgcgaaattaaactgga | |
| gggccccgtcagccctgatgtggagcctggcaaagaggagaccgaggaaa | |
| gcaaaaaacgcaagcgtaaaccatatcggcctggcattggtggtttcatg | |
| gtgcgacagcggaaatcccacacacgcacgaaaaaggggcctgctgcaca | |
| ggcggaggtgttgagtggggatgggcagcccgacgaggtgatacctgctg | |
| acctgcctgcagagggcgccgtggagcagagcttagctgaaggggatgag | |
| aagaagaagcaacagcggcgagggcgcaagaagagcaaactggaggacat | |
| gttccctgcttacttgcaggaagccttctttgggaaggagctgctggacc | |
| tgagccgtaaggccctttttgcagttggggtgggccggccaagctttgga | |
| ctagggaccccaaaagccaagggagatggaggctcagaaaggaaggaact | |
| ccccacatcgcagaaaggagatgatggtccagatattgcagatgaagaat | |
| cccgtggcctcgagggcaaagccgatacaccaggacctgaggatgggggc | |
| gtgaaggcatccccagtgcccagtgaccctgagaagccaggcaccccagg | |
| tgaagggatgcttagctctgacttagacaggatttccacagaagaactgc | |
| ccaagatggaatccaaggacctgcagcagctcttcaaggatgttctgggc | |
| tctgaacgagaacagcatctgggttgtggaacccctggcctagaaggcag | |
| ccgtacgccactgcagaggccctttcttcaaggtggactccctttgggca | |
| atctgccctccagcagcccaatggactcctacccaggcctctgccagtcc | |
| ccgttcctggattctagggagcgcgggggcttctttagcccggaacccgg | |
| tgagcccgacagcccctggacgggctcaggtggcaccacgccctccaccc | |
| ccacaacccccaccacggagggtgagggcgacggactctcctataaccag | |
| cggagtcttcagcgctgggagaaggatgaggagttgggccagctgtccac | |
| catctcacctgtgctctatgccaacattaattttcctaatctcaagcaag | |
| actacccagactggtcaagccgttgcaaacaaatcatgaagctctggaga | |
| aaggttccagcagctgacaaagccccctacctgcaaaaggccaaagataa | |
| ccgggcagctcaccgcatcaacaaggtgcagaagcaggctgagagccaga | |
| tcaacaagcagaccaaggtgggcgacatagcccgtaagactgaccgaccg | |
| gccctacatctccgcattcccccgcagccaggggcactgggcagcccgcc | |
| ccccgctgctgcccccaccattttcattggcagccccactacccccgccg | |
| gcttgtctacctctgcggacgggttcctgaagccgccggcgggctcggtg | |
| cctggccctgactcgcctggtgagctcttcctcaagctcccaccccaggt | |
| gcccgcccaagtgccttcgcaggacccctttggactggcccctgcctatc | |
| ccctggagccccgcttccccacggcaccgcccacctatcccccctatcct | |
| agtcctacgggggcccctgcgcagcccccgatgctgggcgcctcatctcg | |
| tcctggggctggccagccaggggaattccacactaccccacctggcaccc | |
| ccagacaccagccctccacacctgacccattcctcaaaccccgctgcccc | |
| tcgctggataacttggctgtgcctgagagccctggggtagggggaggcaa | |
| agcttccgagcccctgctctcgcccccaccttttggggagtcccggaagg | |
| ccctagaggtgaagaaggaagagcttggggcatcctctcctagctatggg | |
| cccccaaacctgggctttgttgactcaccctcctcaggcacccacctggg | |
| tggcctggagttaaagacacctgatgtcttcaaagcccccctgacccctc | |
| gggcatctcaggtagagccccagagcccgggcttgggcctaaggccccag | |
| gagccaccccctgcccaggctttggcaccttctcctccaagtcacccaga | |
| catctttcgccctggctcctacactgacccatatgctcagcccccattga | |
| ctcctcggccccaacctccgccccctgagagctgctgtgctctgccccct | |
| cgctcactgccctccgaccctttctcccgagtgcctgccagtcctcagtc | |
| ccagtccagctcccagtctccactgacaccccggcctctgtctgctgaag | |
| ctttttgcccatcacccgttacccctcgcttccagtcccctgacccttat | |
| tctcgcccaccctcacgccctcagtcccgtgacccatttgccccattgca | |
| taagccaccccgaccccagccccctgaagttgcctttaaggctgggtctc | |
| tagcccacacttcgctgggggctggggggttcccagcagccctgcccgcg | |
| gggccagcaggtgagctccatgccaaggtcccaagtgggcagccccccaa | |
| ttttgtccggtcccctgggacgggtgcatttgtgggcaccccctctccca | |
| tgcgtttcactttccctcaggcagtaggggagccttccctaaagccccct | |
| gtccctcagcctggtctcccgccaccccatgggatcaacagccattttgg | |
| gcccggccccaccttgggcaagcctcaaagcacaaactacacagtagcca | |
| cagggaacttccacccatcgggcagccccctggggcccagcagcgggtcc | |
| acaggggagagctatgggctgtccccactacgccctccgtcggttctgcc | |
| accacctgcacccgacggatccctcccctacctgtcccatggagcctcac | |
| agcgatcaggcatcacctctcctgtcgaaaagcgagaagacccagggact | |
| ggaatgggtagctctttggcgacagctgaactcccaggtacccaggaccc | |
| aggcatgtccggccttagccaaacagagctggagaagcaacggcagcgcc | |
| agcgactacgagagctgctgattcggcagcagatccagcgcaacaccctg | |
| cggcaggagaaggaaacagctgcagcagctgcaggagcagtggggcctcc | |
| aggcagctggggtgctgagcccagcagccctgcctttgagcagctgagtc | |
| gaggccagaccccctttgctgggacacaggacaagagcagccttgtgggg | |
| ttgcccccaagcaagctgagtggccccatcctggggccagggtccttccc | |
| tagcgatgaccgactctcccggccacctccaccagccacgccttcctcta | |
| tggatgtgaacagccggcaactggtaggaggctcccaagctttctatcag | |
| cgagcaccctatcctgggtccctgcccttacagcagcaacagcaacaact | |
| gtggcagcaacaacaggcaacagcagcaacctccatgcgatttgccatgt | |
| cagctcgctttccatcaactcctggacctgaacttggccgccaagcccta | |
| ggttccccgttggcgggaatttccacccgtctgccaggccctggtgagcc | |
| agtgcctggtccagctggtcctgcccagttcattgagctgcggcacaatg | |
| tacagaaaggactgggacctgggggcactccgtttcctggtcagggccca | |
| cctcagagaccccgtttttaccctgtaagtgaggacccccaccgactggc | |
| tcctgaagggcttcggggcctggcggtatcaggtcttcccccacagaaac | |
| cctcagccccaccggcccctgaattgaacaacagtcttcatccaacaccc | |
| cacaccaagggtcctaccctgccaactggtttggagctggtcaaccggcc | |
| cccgtcgagcactgagcttggccgccccaatcctctggccctggaagctg | |
| ggaagttgccctgtgaggatcccgagctggatgacgattttgatgcccac | |
| aaggccctagaggatgatgaagagcttgctcacctgggtctgggtgtgga | |
| tgtggccaagggtgatgatgaacttggcaccttagaaaacctggagacca | |
| atgacccccacttggatgacctgctcaatggagacgagtttgacctgctg | |
| gcatatactgatcctgagctggacactggggacaagaaggatatcttcaa | |
| tgagcacctgaggctggtagaatcggctaatgagaaggctgaacgggagg | |
| ccctgctgcggggggtggagccaggacccttgggccctgaggagcgccct | |
| ccccctgctgctgatgcctctgaaccccgcctggcatctgtgctccctga | |
| ggtgaagcccaaggtggaggagggtggacgccacccttctccttgccaat | |
| tcaccattgctacccccaaggtagagcccgcacctgctgccaattccctt | |
| ggcctggggctaaagccaggacagagcatgatgggcagccgggatacccg | |
| gatgggcacagggccattttctagcagtgggcacacagctgagaaggcct | |
| cctttggggccacgggaggaccaccagctcacctgctgacccccagccca | |
| ctgagtggcccaggaggatcctccctgctggaaaagtttgagctcgagag | |
| tggggctttgaccttgcctggtggacctgcagcatctggggatgagctag | |
| acaagatggagagctcactggtagccagcgagttacccctgctcattgag | |
| gacctgttggagcatgagaagaaggagctgcagaagaagcagcagctttc | |
| agcacagttgcagcctgcccagcagcagcagcaacagcagcagcagcatt | |
| ccctactgtctgcaccaggccctgcccaggccatgtctttgccacatgag | |
| ggctcttctcccagtttggctgggtcccaacagcagctttccctgggtct | |
| tgcaggtgcccgacagccaggtttgccccagccactgatgcccacccagc | |
| caccagctcatgccctccagcaacgcctggctccatccatggctatggtg | |
| tccaatcaagggcatatgctaagtgggcagcatggagggcaggcaggctt | |
| ggtaccccagcagagctcacagccagtgctatcacagaagcccatgggca | |
| ccatgccaccttccatgtgcatgaagccgcagcaattggcaatgcagcag | |
| cagctggcaaacagcttcttcccagatacagacctggacaaatttgctgc | |
| agaagatatcattgatcccattgcaaaggccaagatggtggctttgaaag | |
| gcatcaagaaagtgatggctcagggcagcattggggtggcacctggtatg | |
| aacagacagcaagtgtctctgctagcccagaggctctcggggggacctag | |
| cagtgatctgcagaaccatgtggcagctgggagtggccaggagcggagtg | |
| ctggtgatccctcccagcctcgtcccaacccgcccacttttgctcaggga | |
| gtgatcaatgaagctgaccagcggcagtatgaggagtggctgttccatac | |
| ccagcagctcctacagatgcagctgaaggtgctagaggagcagattggtg | |
| tacaccgcaagtcccggaaggctctgtgtgccaagcagcgcactgccaaa | |
| aaagctggccgtgagttcccagaagctgatgctgagaagctcaagctggt | |
| tacagagcagcagagcaagatccagaaacaactggatcaggtccggaaac | |
| agcagaaggagcacactaatctcatggcagaatatcggaacaagcagcag | |
| caacaacagcagcagcagcagcaacaacagcaacagcactcagctgtgct | |
| ggctctcagcccttcccagagtccccggctgctcaccaagctccctggtc | |
| agctgctccctggccatgggctgcagccaccacaggggcctccgggtggg | |
| caagccggaggtcttcgcctgacccctgggggtatggcactacctggaca | |
| gcctggtggccccttccttaatacagctctggcccaacagcagcaacagc | |
| aacattctggtggggctggatccctggctggcccttcagggggcttcttc | |
| cctggcaaccttgctcttcgaagcctcggacctgattcaaggcttttaca | |
| ggaaaggcagctgcagctgcagcagcaacgtatgcagctggcccagaaac | |
| tgcagcagcagcagcagcagcaacagcagcagcagcaccttctaggacag | |
| gtggcaatccagcagcaacagcagcagggtcctggagtacagacaaacca | |
| agctctgggtcccaagccccagggccttatgcctcccagcagccaccaag | |
| gcctcctggtccagcagctgtcccctcaaccaccccaggggccccagggc | |
| atgctgggccctgcccaggtggctgtgttgcagcagcagcaccctggagc | |
| tttgggcccccagggccctcacagacaggtgcttatgacccagtcccggg | |
| tgctcagttccccccagctggcacagcagggtcagggccttatgggacac | |
| aggctggtcacagcccagcagcagcagcagcaacaacagcaccaacagca | |
| agggtccatggcagggctgtcccatcttcagcagagtctgatgtcacaca | |
| gtgggcagcccaaactgagcgctcagcccatgggctctttacagcagctt | |
| cagcagcagcagcagctgcaacagcaacagcaacttcagcagcagcagca | |
| gcagcagctacaacagcaacagcaacttcagcagcaacagcttcaacagc | |
| agcaacagcagcagcagcttcaacaacagcagcagcaacagcttcaacag | |
| cagcaacagcagctacaacagcaacagcaacaacaacagcagcagtttca | |
| acagcagcagcaacagcagcagatgggccttttaaaccagagtcgaactt | |
| tactgtctcctcagcaacaacagcagcagcaagtggcacttggccctggc | |
| atgccagcaaagcctcttcaacacttttctagccctggagccctgggtcc | |
| aaccctcctcctgacgggcaaggaacaaaacaccgtagacccagccgttt | |
| cttcagaggccactgaggggccctctacacatcagggagggccgttagca | |
| ataggaactacccctgagtcaatggccactgaaccaggagaggtaaagcc | |
| ctcactctctggggactcacaactcctgcttgtccaaccccagccccagc | |
| ctcagcccagctctctgcagctgcagccacctctgaggcttccaggacaa | |
| cagcagcagcaagttagcctgctccacacagcaggtggaggaagccatgg | |
| gcagctaggcagtggatcatcttctgaggcctcatctgtgccccacctgc | |
| tggctcagccctctgtttccttaggggatcagcctgggtccatgacccag | |
| aaccttctgggcccccaacagcccatgctagagcggcccatgcaaaataa | |
| tacagggccacaacctcccaaaccaggacctgtcctccagtctgggcagg | |
| gtctgcctggggttggaatcatgcctacggtgggtcagcttcgagcacag | |
| ctccaaggagtcctggccaaaaacccacagctgcggcacttaagtcctca | |
| gcagcagcagcagctacaggcactcctcatgcagcggcagctgcagcaga | |
| gtcaggcagtacgccagaccccaccctaccaggagcctgggacccagacc | |
| tctcccctccagggcctcctgggctgccaacctcaacttgggggcttccc | |
| tggaccacagacaggccccctccaggagctaggggcagggcctcgacctc | |
| agggcccaccccggctccctgccccaccaggagccttatctacaggacca | |
| gtccttggccctgtccatcccacacctccaccatccagccctcaagagcc | |
| aaagagaccttcacaattaccttcccccagctcccagcttcccactgagg | |
| cccagctccctcccacccatccagggacccccaaacctcaggggccaacc | |
| ttggagccgcctcctgggagggtctcacctgctgctgcccagcttgcaga | |
| taccttgtttagcaagggtctgggaccttgggatcccccagacaacctag | |
| cagaaacccagaagccagagcagagcagcctggtacctgggcatctggac | |
| caggtgaatggacaggtggtgcctgaggcatcccaactcagcatcaagca | |
| ggaacctcgggaagagccatgtgccctgggagcccagtcagtgaagaggg | |
| aggccaatggggagccaataggggcaccaggaaccagcaaccacctcctg | |
| ctggcaggccctcgctcagaagctgggcatctgctcttgcagaagctact | |
| ccgggcaaagaatgtgcaactcagcactgggcgggggtccgaggggctgc | |
| gagctgagatcaacgggcacattgacagcaagctggctgggctggagcag | |
| aaactacagggtacccccagcaacaaggaggatgcagcagcaaggaagcc | |
| tttgacaccgaagcccaagcgggtacagaaggcaagcgacaggttggtga | |
| gctcccgaaagaagctgcggaaggaggacggggtcagggccagcgaggcc | |
| ttgctgaaacagctgaaacaggagctgtccctgctgcccctaacggagcc | |
| tgctatcaccgccaattttagcctctttgccccctttggcagtggctgcc | |
| cagtcaatgggcagagccagctgaggggggcctttggaagtggggcgctg | |
| cccactggccctgactactattcccagctgcttaccaagaataacctgag | |
| taacccgccgacaccaccctcgtcgctgccccccaccccacccccatcgg | |
| tgcagcagaagatggtgaatggcgtcaccccatctgaagagctgggggag | |
| caccccaaggatgctgcctctgcccgggatagtgaaagggcactgaggga | |
| tacttcagaggtgaagagtctagacctgctggctgccttgcctacacccc | |
| ctcacaatcagactgaggatgtcaggatggagagtgatgaggatagcgat | |
| tctcctgacagcattgtgccagcttcatcccctgagagcatcttggggga | |
| ggaggcccctcgtttccctcatctgggctcaggccggtgggagcaagagg | |
| accgggccctctcccctgtcatccccctcattcctcgggccagcatccca | |
| gtcttcccagataccaaaccttatggggcccttggcctggaggtccctgg | |
| aaagctgcctgtcacaacttgggaaaagggcaaaggaagtgaggtgtcag | |
| tcatgctcacagtctctgctgctgcagccaagaacctgaatggcgtgatg | |
| gtggcagtggcggagctgctgagcatgaagatccccaactcctatgaggt | |
| gctgttcccagagagccccgcccgggcaggcactgagccaaagaaggggg | |
| aagctgagggtcctggtgggaaggaaaagggtctggaaggcaagagccca | |
| gacactggccctgattggctgaagcagtttgatgcagtgttgcctggcta | |
| taccctgaagagccaactagacatcttgagcctcctgaaacaggagagcc | |
| ccgccccagagccacccactcagcacagctatacctacaatgtctccaat | |
| ctggatgtgcgacagctctcggccccacctcctgaagaaccctccccgcc | |
| cccttcccccttggcaccttctcctgccagtccccctactgagcccttgg | |
| ttgaacttcccaccgaacccttggctgagccacccgtcccctcacctctg | |
| ccactggcctcatcccctgaatcagcccgacccaagccccgtgcccggcc | |
| ccctgaagaaggtgaagattcccgtcctcctcgcctcaagaaatggaaag | |
| gagtgcgctggaagcggcttcggctgctgctgaccatccagaagggcagt | |
| gggcggcaggaggatgagcgggaagtggcagagtttatggagcagcttgg | |
| cacagccttgcgacctgacaaggtaccgcgagacatgcgtcgctgctgtt | |
| tctgtcatgaggagggtgacggggccactgatgggcctgcccgtctgctg | |
| aacctggacctggacctgtgggtgcacctcaactgtgccctttggtccac | |
| ggaggtgtatgagacccagggcggggcactgatgaatgtggaggttgccc | |
| tgcaccgaggactgctaaccaagtgctccctgtgccagcgaactggtgcc | |
| accagcagctgcaatcgcatgcgttgccccaatgtctaccattttgcttg | |
| tgccatccgtgccaagtgcatgttcttcaaggacaagaccatgctgtgtc | |
| caatgcataagatcaaggggccctgtgagcaagagctgagctcttttgct | |
| gtcttccggcgggtctacattgagcgggacgaggtgaagcaaatcgctag | |
| catcattcagcggggagaacggctgcacatgttccgtgtggggggccttg | |
| tgttccacgccatcggacagctgctgcctcaccagatggctgactttcat | |
| agtgccactgccctctatcccgtgggctacgaggccacgcgcatctattg | |
| gagcctccgcaccaacaatcgtcgctgctgctatcgctgttctattggtg | |
| agaacaacgggcggccggagtttgtaatcaaagtcatcgagcagggcctg | |
| gaggacctggtcttcactgacgcctctccccaggccgtgtggaatcgcat | |
| cattgagcctgtggctgccatgagaaaagaggctgacatgctgcgactct | |
| tccctgagtatctgaagggcgaggagctctttgggctgacggtgcatgcc | |
| gtgcttcgcatagctgaatcactgcccggggtggagagctgtcaaaacta | |
| tttattccgctatgggcgccacccccttatggagctgccactcatgatca | |
| accccactggctgtgcccgatcagagcctaaaatcctcacacactacaaa | |
| cggccccataccctgaacagcaccagcatgtctaaggcatatcagagcac | |
| cttcacaggcgagaccaacaccccctacagcaagcagtttgtgcactcca | |
| agtcatctcagtaccggcggctgcgcaccgaatggaagaacaacgtgtac | |
| ctggctcgctcccgtatccagggcctggggctctatgcagccaaggacct | |
| agaaaagcacacaatggttatcgagtacattggcaccatcattcggaacg | |
| aggtggccaaccggcgggagaaaatctacgaagagcagaatcgaggcatc | |
| tacatgttccgaataaacaatgaacatgtgattgatgctacgttgaccgg | |
| cggccctgccaggtacattaaccattcctgtgcccctaactgtgtggccg | |
| aagtcgtgacatttgacaaagaggacaaaatcatcatcatctccagccgg | |
| cgaatccccaaaggagaggagctaacctatgactatcagtttgattttga | |
| ggacgatcagcacaagatcccctgccactgtggagcctggaattgtcgga | |
| aatggatgaactaa | |
| EP300 | SEQ ID |
| atggccgagaatgtggtggaaccggggccgccttcagccaagcggcctaa | NO: 335 |
| actctcatctccggccctctcggcgtccgccagcgatggcacagattttg | |
| gctctctatttgacttggagcacgacttaccagatgaattaatcaactct | |
| acagaattgggactaaccaatggtggtgatattaatcagcttcagacaag | |
| tcttggcatggtacaagatgcagcttctaaacataaacagctgtcagaat | |
| tgctgcgatctggtagttcccctaacctcaatatgggagttggtggccca | |
| ggtcaagtcatggccagccaggcccaacagagcagtcctggattaggttt | |
| gataaatagcatggtcaaaagcccaatgacacaggcaggcttgacttctc | |
| ccaacatggggatgggcactagtggaccaaatcagggtcctacgcagtca | |
| acaggtatgatgaacagtccagtaaatcagcctgccatgggaatgaacac | |
| agggatgaatgcgggcatgaatcctggaatgttggctgcaggcaatggac | |
| aagggataatgcctaatcaagtcatgaacggttcaattggagcaggccga | |
| gggcgacagaatatgcagtacccaaacccaggcatgggaagtgctggcaa | |
| cttactgactgagcctcttcagcagggctctccccagatgggaggacaaa | |
| caggattgagaggcccccagcctcttaagatgggaatgatgaacaacccc | |
| aatccttatggttcaccatatactcagaatcctggacagcagattggagc | |
| cagtggccttggtctccagattcagacaaaaactgtactatcaaataact | |
| tatctccatttgctatggacaaaaaggcagttcctggtggaggaatgccc | |
| aacatgggtcaacagccagccccgcaggtccagcagccaggcctggtgac | |
| tccagttgcccaagggatgggttctggagcacatacagctgatccagaga | |
| agcgcaagctcatccagcagcagcttgttctccttttgcatgctcacaag | |
| tgccagcgccgggaacaggccaatggggaagtgaggcagtgcaaccttcc | |
| ccactgtcgcacaatgaagaatgtcctaaaccacatgacacactgccagt | |
| caggcaagtcttgccaagtggcacactgtgcatcttctcgacaaatcatt | |
| tcacactggaagaattgtacaagacatgattgtcctgtgtgtctccccct | |
| caaaaatgctggtgataagagaaatcaacagccaattttgactggagcac | |
| ccgttggacttggaaatcctagctctctaggggtgggtcaacagtctgcc | |
| cccaacctaagcactgttagtcagattgatcccagctccatagaaagagc | |
| ctatgcagctcttggactaccctatcaagtaaatcagatgccgacacaac | |
| cccaggtgcaagcaaagaaccagcagaatcagcagcctgggcagtctccc | |
| caaggcatgcggcccatgagcaacatgagtgctagtcctatgggagtaaa | |
| tggaggtgtaggagttcaaacgccgagtcttctttctgactcaatgttgc | |
| attcagccataaattctcaaaacccaatgatgagtgaaaatgccagtgtg | |
| ccctccctgggtcctatgccaacagcagctcaaccatccactactggaat | |
| tcggaaacagtggcacgaagatattactcaggatcttcgaaatcatcttg | |
| ttcacaaactcgtccaagccatatttcctacgccggatcctgctgcttta | |
| aaagacagacggatggaaaacctagttgcatatgctcggaaagttgaagg | |
| ggacatgtatgaatctgcaaacaatcgagcggaatactaccaccttctag | |
| ctgagaaaatctataagatccagaaagaactagaagaaaaacgaaggacc | |
| agactacagaagcagaacatgctaccaaatgctgcaggcatggttccagt | |
| ttccatgaatccagggcctaacatgggacagccgcaaccaggaatgactt | |
| ctaatggccctctacctgacccaagtatgatccgtggcagtgtgccaaac | |
| cagatgatgcctcgaataactccacaatctggtttgaatcaatttggcca | |
| gatgagcatggcccagccccctattgtaccccggcaaacccctcctcttc | |
| agcaccatggacagttggctcaacctggagctctcaacccgcctatgggc | |
| tatgggcctcgtatgcaacagccttccaaccagggccagttccttcctca | |
| gactcagttcccatcacagggaatgaatgtaacaaatatccctttggctc | |
| cgtccagcggtcaagctccagtgtctcaagcacaaatgtctagttcttcc | |
| tgcccggtgaactctcctataatgcctccagggtctcaggggagccacat | |
| tcactgtccccagcttcctcaaccagctcttcatcagaattcaccctcgc | |
| ctgtacctagtcgtacccccacccctcaccatactcccccaagcataggg | |
| gctcagcagccaccagcaacaacaattccagcccctgttcctacacctcc | |
| tgccatgccacctgggccacagtcccaggctctacatccccctccaaggc | |
| agacacctacaccaccaacaacacaacttccccaacaagtgcagccttca | |
| cttcctgctgcaccttctgctgaccagccccagcagcagcctcgctcaca | |
| gcagagcacagcagcgtctgttcctaccccaacagcaccgctgcttcctc | |
| cgcagcctgcaactccactttcccagccagctgtaagcattgaaggacag | |
| gtatcaaatcctccatctactagtagcacagaagtgaattctcaggccat | |
| tgctgagaagcagccttcccaggaagtgaagatggaggccaaaatggaag | |
| tggatcaaccagaaccagcagatactcagccggaggatatttcagagtct | |
| aaagtggaagactgtaaaatggaatctaccgaaacagaagagagaagcac | |
| tgagttaaaaactgaaataaaagaggaggaagaccagccaagtacttcag | |
| ctacccagtcatctccggctccaggacagtcaaagaaaaagattttcaaa | |
| ccagaagaactacgacaggcactgatgccaactttggaggcactttaccg | |
| tcaggatccagaatcccttccctttcgtcaacctgtggaccctcagcttt | |
| taggaatccctgattactttgatattgtgaagagccccatggatctttct | |
| accattaagaggaagttagacactggacagtatcaggagccctggcagta | |
| tgtcgatgatatttggcttatgttcaataatgcctggttatataaccgga | |
| aaacatcacgggtatacaaatactgctccaagctctctgaggtctttgaa | |
| caagaaattgacccagtgatgcaaagccttggatactgttgtggcagaaa | |
| gttggagttctctccacagacactgtgttgctacggcaaacagttgtgca | |
| caatacctcgtgatgccacttattacagttaccagaacaggtatcatttc | |
| tgtgagaagtgtttcaatgagatccaaggggagagcgtttctttggggga | |
| tgacccttcccagcctcaaactacaataaataaagaacaattttccaaga | |
| gaaaaaatgacacactggatcctgaactgtttgttgaatgtacagagtgc | |
| ggaagaaagatgcatcagatctgtgtccttcaccatgagatcatctggcc | |
| tgctggattcgtctgtgatggctgtttaaagaaaagtgcacgaactagga | |
| aagaaaataagttttctgctaaaaggttgccatctaccagacttggcacc | |
| tttctagagaatcgtgtgaatgactttctgaggcgacagaatcaccctga | |
| gtcaggagaggtcactgttagagtagttcatgcttctgacaaaaccgtgg | |
| aagtaaaaccaggcatgaaagcaaggtttgtggacagtggagagatggca | |
| gaatcctttccataccgaaccaaagccctctttgcctttgaagaaattga | |
| tggtgttgacctgtgcttctttggcatgcatgttcaagagtatggctctg | |
| actgccctccacccaaccagaggagagtatacatatcttacctcgatagt | |
| gttcatttcttccgtcctaaatgcttgaggactgcagtctatcatgaaat | |
| cctaattggatatttagaatatgtcaagaaattaggttacacaacagggc | |
| atatttgggcatgtccaccaagtgagggagatgattatatcttccattgc | |
| catcctcctgaccagaagatacccaagcccaagcgactgcaggaatggta | |
| caaaaaaatgcttgacaaggctgtatcagagcgtattgtccatgactaca | |
| aggatatttttaaacaagctactgaagatagattaacaagtgcaaaggaa | |
| ttgccttatttcgagggtgatttctggcccaatgttctggaagaaagcat | |
| taaggaactggaacaggaggaagaagagagaaaacgagaggaaaacacca | |
| gcaatgaaagcacagatgtgaccaagggagacagcaaaaatgctaaaaag | |
| aagaataataagaaaaccagcaaaaataagagcagcctgagtaggggcaa | |
| caagaagaaacccgggatgcccaatgtatctaacgacctctcacagaaac | |
| tatatgccaccatggagaagcataaagaggtcttctttgtgatccgcctc | |
| attgctggccctgctgccaactccctgcctcccattgttgatcctgatcc | |
| tctcatcccctgcgatctgatggatggtcgggatgcgtttctcacgctgg | |
| caagggacaagcacctggagttctcttcactccgaagagcccagtggtcc | |
| accatgtgcatgctggtggagctgcacacgcagagccaggaccgctttgt | |
| ctacacctgcaatgaatgcaagcaccatgtggagacacgctggcactgta | |
| ctgtctgtgaggattatgacttgtgtatcacctgctataacactaaaaac | |
| catgaccacaaaatggagaaactaggccttggcttagatgatgagagcaa | |
| caaccagcaggctgcagccacccagagcccaggcgattctcgccgcctga | |
| gtatccagcgctgcatccagtctctggtccatgcttgccagtgtcggaat | |
| gccaattgctcactgccatcctgccagaagatgaagcgggttgtgcagca | |
| taccaagggttgcaaacggaaaaccaatggcgggtgccccatctgcaagc | |
| agctcattgccctctgctgctaccatgccaagcactgccaggagaacaaa | |
| tgcccggtgccgttctgcctaaacatcaagcagaagctccggcagcaaca | |
| gctgcagcaccgactacagcaggcccaaatgcttcgcaggaggatggcca | |
| gcatgcagcggactggtgtggttgggcagcaacagggcctcccttccccc | |
| actcctgccactccaacgacaccaactggccaacagccaaccaccccgca | |
| gacgccccagcccacttctcagcctcagcctacccctcccaatagcatgc | |
| caccctacttgcccaggactcaagctgctggccctgtgtcccagggtaag | |
| gcagcaggccaggtgacccctccaacccctcctcagactgctcagccacc | |
| ccttccagggcccccacctgcagcagtggaaatggcaatgcagattcaga | |
| gagcagcggagacgcagcgccagatggcccacgtgcaaatttttcaaagg | |
| ccaatccaacaccagatgcccccgatgactcccatggcccccatgggtat | |
| gaacccacctcccatgaccagaggtcccagtgggcatttggagccaggga | |
| tgggaccgacagggatgcagcaacagccaccctggagccaaggaggattg | |
| cctcagccccagcaactacagtctgggatgccaaggccagccatgatgtc | |
| agtggcccagcatggtcaacctttgaacatggctccacaaccaggattgg | |
| gccaggtaggtatcagcccactcaaaccaggcactgtgtctcaacaagcc | |
| ttacaaaaccttttgcggactctcaggtctcccagctctcccctgcagca | |
| gcaacaggtgcttagtatccttcacgccaacccccagctgttggctgcat | |
| tcatcaagcagcgggctgccaagtatgccaactctaatccacaacccatc | |
| cctgggcagcctggcatgccccaggggcagccagggctacagccacctac | |
| catgccaggtcagcagggggtccactccaatccagccatgcagaacatga | |
| atccaatgcaggcgggcgttcagagggctggcctgccccagcagcaacca | |
| cagcagcaactccagccacccatgggagggatgagcccccaggctcagca | |
| gatgaacatgaaccacaacaccatgccttcacaattccgagacatcttga | |
| gacgacagcaaatgatgcaacagcagcagcaacagggagcagggccagga | |
| ataggccctggaatggccaaccataaccagttccagcaaccccaaggagt | |
| tggctacccaccacagcagcagcagcggatgcagcatcacatgcaacaga | |
| tgcaacaaggaaatatgggacagataggccagcttccccaggccttggga | |
| gcagaggcaggtgccagtctacaggcctatcagcagcgactccttcagca | |
| acagatggggtcccctgttcagcccaaccccatgagcccccagcagcata | |
| tgctcccaaatcaggcccagtccccacacctacaaggccagcagatccct | |
| aattctctctccaatcaagtgcgctctccccagcctgtcccttctccacg | |
| gccacagtcccagcccccccactccagtccttccccaaggatgcagcctc | |
| agccttctccacaccacgtttccccacagacaagttccccacatcctgga | |
| ctggtagctgcccaggccaaccccatggaacaagggcattttgccagccc | |
| ggaccagaattcaatgctttctcagcttgctagcaatccaggcatggcaa | |
| acctccatggtgcaagcgccacggacctgggactcagcaccgataactca | |
| gacttgaattcaaacctctcacagagtacactagacatacactag | |
| IIRAS | SEQ ID |
| atgacggaatataagctggtggtggtgggcgccggcggtgtgggcaagag | NO: 336 |
| tgcgctgaccatccagctgatccagaaccattttgtggacgaatacgacc | |
| ccactatagaggattcctaccggaagcaggtggtcattgatggggagacg | |
| tgcctgttggacatcctggataccgccggccaggaggagtacagcgccat | |
| gcgggaccagtacatgcgcaccggggagggcttcctgtgtgtgtttgcca | |
| tcaacaacaccaagtcttttgaggacatccaccagtacagggagcagatc | |
| aaacgggtgaaggactcggatgacgtgcccatggtgctggtggggaacaa | |
| gtgtgacctggctgcacgcactgtggaatctcggcaggctcaggacctcg | |
| cccgaagctacggcatcccctacatcgagacctcggccaagacccggcag | |
| ggcagccgctctggctctagctccagctccgggaccctctgggacccccc | |
| gggacccatgtga | |
| KEAP1 | SEQ ID |
| atgcagccagatcccaggcctagcggggctggggcctgctgccgattcct | NO: 337 |
| gcccctgcagtcacagtgccctgagggggcaggggacgcggtgatgtacg | |
| cctccactgagtgcaaggcggaggtgacgccctcccagcatggcaaccgc | |
| accttcagctacaccctggaggatcataccaagcaggcctttggcatcat | |
| gaacgagctgcggctcagccagcagctgtgtgacgtcacactgcaggtca | |
| agtaccaggatgcaccggccgcccagttcatggcccacaaggtggtgctg | |
| gcctcatccagccctgtcttcaaggccatgttcaccaacgggctgcggga | |
| gcagggcatggaggtggtgtccattgagggtatccaccccaaggtcatgg | |
| agcgcctcattgaattcgcctacacggcctccatctccatgggcgagaag | |
| tgtgtcctccacgtcatgaacggtgctgtcatgtaccagatcgacagcgt | |
| tgtccgtgcctgcagtgacttcctggtgcagcagctggaccccagcaatg | |
| ccatcggcatcgccaacttcgctgagcagattggctgtgtggagttgcac | |
| cagcgtgcccgggagtacatctacatgcattttggggaggtggccaagca | |
| agaggagttcttcaacctgtcccactgccaactggtgaccctcatcagcc | |
| gggacgacctgaacgtgcgctgcgagtccgaggtcttccacgcctgcatc | |
| aactgggtcaagtacgactgcgaacagcgacggttctacgtccaggcgct | |
| gctgcgggccgtgcgctgccactcgttgacgccgaacttcctgcagatgc | |
| agctgcagaagtgcgagatcctgcagtccgactcccgctgcaaggactac | |
| ctggtcaagatcttcgaggagctcaccctgcacaagcccacgcaggtgat | |
| gccctgccgggcgcccaaggtgggccgcctgatctacaccgcgggcggct | |
| acttccgacagtcgctcagctacctggaggcttacaaccccagtgacggc | |
| acctggctccggttggcggacctgcaggtgccgcggagcggcctggccgg | |
| ctgcgtggtgggcgggctgttgtacgccgtgggcggcaggaacaactcgc | |
| ccgacggcaacaccgactccagcgccctggactgttacaaccccatgacc | |
| aatcagtggtcgccctgcgcccccatgagcgtgccccgtaaccgcatcgg | |
| ggtgggggtcatcgatggccacatctatgccgtcggcggctcccacggct | |
| gcatccaccacaacagtgtggagaggtatgagccagagcgggatgagtgg | |
| cacttggtggccccaatgctgacacgaaggatcggggtgggcgtggctgt | |
| cctcaatcgtctcctttatgccgtggggggctttgacgggacaaaccgcc | |
| ttaattcagctgagtgttactacccagagaggaacgagtggcgaatgatc | |
| acagcaatgaacaccatccgaagcggggcaggcgtctgcgtcctgcacaa | |
| ctgtatctatgctgctgggggctatgatggtcaggaccagctgaacagcg | |
| tggagcgctacgatgtggaaacagagacgtggactttcgtagcccccatg | |
| aagcaccggcgaagtgccctggggatcactgtccaccaggggagaatcta | |
| cgtccttggaggctatgatggtcacacgttcctggacagtgtggagtgtt | |
| acgacccagatacagacacctggagcgaggtgacccgaatgacatcgggc | |
| cggagtggggtgggcgtggctgtcaccatggagccctgccggaagcagat | |
| tgaccagcagaactgtacctgttga | |
| ZNF750 | SEQ ID |
| atgagtctcctcaaagagcggaagccaaaaaagccacattacatccccag | NO: 338 |
| gcctccaggaaagcccttcaagtataaatgtttccaatgtccctttactt | |
| gcaatgagaagtcacatctttttaatcacatgaagtatggtctttgtaaa | |
| aactcgattactttagtatcagagcaggatcgagttcccaagtgccctaa | |
| atctaactcactagaccccaagcaaaccaaccagcccgatgccacggcga | |
| agccagcctcttccaagtctgtcgcaaatggactctctgccttcgactcg | |
| aagcttcagcacagctctgccagggaagacatcaaggaaaacctggagct | |
| gcaagcccggggaacccacaggtgcctgggacagaagccagccctccaca | |
| gggcatcaccctgcaagagcccagctccggaagccgccctcggtgcccag | |
| cctgctctggaaggcgcagctcggccttctgcatttgttccagtcggcga | |
| gcacagactcaaggggccagacaacgccgaggcgcccgagacactggctt | |
| tacacaaccccactgccaaggccgtgtctttccacaccaagtcggccttc | |
| cacactcctggctacccctggaaagccggctcacctttccttccaccaga | |
| gtttccacataaaatctcatctacaaaggggcttggggccatttcccctt | |
| acatgcaccccacaatcccagagtacccgcctcacttttacacagagcac | |
| gggctggccaccatctactcgccttacctgctggctgggagctcgcctga | |
| gtgtgacgcacccctgctgtcagtctacggaacccaagacccgagacact | |
| tcctgcctcacccggggccgatccctaagcacctggctccatctccagcc | |
| acatacgatcactacaggtttttccagcaatatccctctaacctgccgat | |
| tccttacggattttacaggccagagtctgcattttcctcctatggtctca | |
| gactcccacctgtcactggcctcacccgagatcagagctctcacctgctt | |
| gaagaagccaccctggtctatccagcctcgagtccttccaggttaaaccc | |
| ttcggaccccaacagaaaacacgtcgagttcgaaagtccaattcctgagg | |
| ctaaagactcctccaaggctgggcagagagacacggaagggtccaaaatg | |
| agcccccgcgcagggagtgcagccacgggctccccagggaggccgagccc | |
| caccgacttcatgcagacgagccagacctgcgaaggcctgtacgacctct | |
| ccaacaaggcagcctccagcgcactgggaagactctacccgccagagcaa | |
| agcctcacagccttcaggcctgttaagaaaagcacagaatgcctacctgc | |
| ccaggctgctgagaccacagcagagtctccagtaagcctcaatgttgtga | |
| acggagaccctcctgctccgaccggaagcgcctctctcgtctcggaggcc | |
| gcgccttccagtccggacgacagctccgggatgggccccctcaacctctc | |
| caagaaatcagagataaacctggcagccacccacgaacccacgtaccaag | |
| gcagcccccaggcggaaaccgccagcttctcagagctgcaggaccttcca | |
| ctcaatctctcggtgaaggacccctgtaacacccaggctccgaggcctgc | |
| cttccccggtcgaccacgagctgcagaacctgctgctgctgttccacaga | |
| agactgggacagaaggttctgaggatgggcccagccaccctgagaccaag | |
| ccaggcagcctcgacggtgacggggccccacccacaggccccggcgagga | |
| ggctccagacgcatgcgcggtggacagcagcgaggagcagaagcagacgg | |
| cagccgtggccctgtgccagctggcggcctacagccccaggaacatccgg | |
| gtgggcgatggggatgctgcggccccggaacctgcctgccggcaagacac | |
| acccacactgagctccatggagagccaagaggcccagtgtgacctcagac | |
| ccaaaggacaaaagaggacaagtctaagggatgctggaaaatcccagcaa | |
| ggagctaagaaggcgaagctgcaggacacggccagagtgttcacactacg | |
| aaggagggcccgggtgtcctaa | |
| CCR5 | SEQ ID |
| atggattatcaagtgtcaagtccaatctatgacatcaattattatacatc | NO: 339 |
| ggagccctgccaaaaaatcaatgtgaagcaaatcgcagcccgcctcctgc | |
| ctccgctctactcactggtgttcatctttggttttgtgggcaacatgctg | |
| gtcatcctcatcctgataaactgcaaaaggctgaagagcatgactgacat | |
| ctacctgctcaacctggccatctctgacctgtttttccttcttactgtcc | |
| ccttctgggctcactatgctgccgcccagtgggactttggaaatacaatg | |
| tgtcaactcttgacagggctctattttataggcttcttctctggaatctt | |
| cttcatcatcctcctgacaatcgataggtacctggctgtcgtccatgctg | |
| tgtttgctttaaaagccaggacggtcacctttggggtggtgacaagtgtg | |
| atcacttgggtggtggctgtgtttgcgtctctcccaggaatcatctttac | |
| cagatctcaaaaagaaggtcttcattacacctgcagctctcattttccat | |
| acagtcagtatcaattctggaagaatttccagacattaaagatagtcatc | |
| ttggggctggtcctgccgctgcttgtcatggtcatctgctactcgggaat | |
| cctaaaaactctgcttcggtgtcgaaatgagaagaagaggcacagggctg | |
| tgaggcttatcttcaccatcatgattgtttattttctcttctgggctccc | |
| tacaacattgtccttctcctgaacaccttccaggaattctttggcctgaa | |
| taattgcagtagctctaacaggttggaccaagctatgcaggtgacagaga | |
| ctcttgggatgacgcactgctgcatcaaccccatcatctatgcctttgtc | |
| ggggagaagttcagaaactacctcttagtcttcttccaaaagcacattgc | |
| caaacgcttctgcaaatgctgttctattttccagcaagaggctcccgagc | |
| gagcaagctcagtttacacccgatccactggggagcaggaaatatctgtg | |
| ggcttgtga | |

### REFERENCES

1. Brash D E, et al., A role for sunlight in skin cancer: UV-induced p53 mutations in squamous cell carcinoma. Proc Natl Acad Sci USA. National Academy of Sciences; 1991; 88:10124-8.
2. Blot W J, et al., Smoking and Drinking in Relation to Oral and Pharyngeal Cancer. Cancer Res. 1988; 48:3282-7.
3. Ang K K, et al., Human Papillomavirus and Survival of Patients with Oropharyngeal Cancer. N Engl J Med [Internet]. 2010 [cited 2019 Dec. 10];363:24-35. Available from: nejm.org/doi/abs/10.1056/NEJMoa0912217
4. Bray F, et al., Global cancer statistics 2018: GLOBOCAN estimates of incidence and mortality worldwide for 36 cancers in 185 countries. CA Cancer J Clin. American Cancer Society; 2018; 68:394-424.
5. FDA. Hematology/Oncology (Cancer) Approvals & Safety Notifications 1 FDA [Internet]. 2020 [cited 2020 May 14]. Available from: fda.gov/drugs/resources-information-approved-drugs/hematologyoncology-cancer-approvals-safety-notifications
6. Marur S, et al., Head and Neck Squamous Cell Carcinoma: Update on Epidemiology, Diagnosis, and Treatment. Mayo Clin Proc [Internet]. 2016 [cited 2020 Apr. 14]; 91:386-96. Available from: dx.doi.org/10.1016/j.mayocp.2015.12.017
7. Stransky N, et al., The Mutational Landscape of Head and Neck Squamous Cell Carcinoma. Science (80-) [Internet]. 2011 [cited 2019 Mar. 12]; 333:1157-60. Available from: ncbi.nlm.nih.gov/pubmed/21798893
8. Hammerman P S, et al., Comprehensive genomic characterization of squamous cell lung cancers. Nature. Nature Publishing Group; 2012; 489:519-25.
9. Gao Y B, et al., Genetic landscape of esophageal squamous cell carcinoma. Nat Genet. Nature Publishing Group; 2014; 46:1097-102.
10. Song Y, et al., Identification of genomic alterations in oesophageal squamous cell cancer. Nature. Nature Publishing Group; 2014; 508:91-5.
11. Li Y Y, et al., Genomic analysis of metastatic cutaneous squamous cell carcinoma. Clin Cancer Res. American Association for Cancer Research Inc.; 2015; 21:1447-56.
12. Pickering C R, et al., Mutational landscape of aggressive cutaneous squamous cell carcinoma. Clin Cancer Res [Internet]. American Association for Cancer Research Inc.; 2014 [cited 2019 Dec. 11]; 20:6582-92. Available from: ncbi.nlm.nih.gov/pubmed/25303977
13. Inman G J, et al., The genomic landscape of cutaneous SCC reveals drivers and a novel azathioprine associated mutational signature. Nat Commun. 2018; 9:1-14.
14. Cancer Genome Atlas Network TCGAN. Comprehensive genomic characterization of head and neck squamous cell carcinomas. Nature [Internet]. NIH Public Access; 2015 [cited 2017 Sep. 5]; 517:576-82. Available from: ncbi.nlm.nih.gov/pubmed/25631445
15. Lawrence M S, et al., Mutational heterogeneity in cancer and the search for new cancer-associated genes. Nature [Internet]. NIH Public Access; 2013 [cited 2017 Sep. 5]; 499:214-8. Available from: ncbi.nlm.nih.gov/pubmed/23770567
16. Raynaud F, et al., Pan-cancer inference of intra-tumor heterogeneity reveals associations with different forms of genomic instability. Kool M, editor. PLOS Genet [Internet]. Public Library of Science; 2018 [cited 2020 Apr. 14]; 14:e1007669. Available from: dx.plos.org/10.1371/j ournal.pgen.1007669
17. Martincorena I, et al., High burden and pervasive positive selection of somatic mutations in normal human skin. Science (80-). American Association for the Advancement of Science; 2015; 348:880-6.
18. Martincorena I, et al., Somatic mutant clones colonize the human esophagus with age. Science (80-) [Internet]. American Association for the Advancement of Science; 2018 [cited 2019 Mar. 5]; 362:911-7. Available from: ncbi.nlm.nih.gov/pubmed/30337457
19. Brinkman E K, et al., Easy quantitative assessment of genome editing by sequence trace decomposition. Nucleic Acids Res [Internet]. Oxford University Press; 2014 [cited 2017 Sep. 5]; 42:e168. Available from: ncbi.nlm.nih.gov/pubmed/25300484
20. Ridky T W, et al., Invasive three-dimensional organotypic neoplasia from multiple normal human epithelia. Nat Med. Nature Publishing Group; 2010; 16:1450-6.
21. Lin D C, et al., Genomic and molecular characterization of esophageal squamous cell carcinoma. Nat Genet. Nature Publishing Group; 2014; 46:467-73.
22. Burk R D, et al., Integrated genomic and molecular characterization of cervical cancer.

Nature. Nature Publishing Group; 2017; 543:378-84.
23. Ratushny V, et al., From keratinocyte to cancer: The pathogenesis and modeling of cutaneous squamous cell carcinoma. J. Clin. Invest. American Society for Clinical Investigation; 2012. page 464-72.
24. Freije A, et al., Inactivation of p53 in Human Keratinocytes Leads to Squamous

Differentiation and Shedding via Replication Stress and Mitotic Slippage. Cell Rep. Elsevier; 2014; 9:1349-60.
25. Pacifico A, et al., Role of p53 and CDKN2A Inactivation in Human Squamous Cell Carcinomas. J Biomed Biotechnol. Hindawi Limited; 2007; 2007.
26. Taylor A M, et al., Genomic and Functional Approaches to Understanding Cancer Aneuploidy. Cancer Cell. Cell Press; 2018; 33:676-689.e3.
27. Dotto G P. Notch tumor suppressor function. Oncogene. Nature Publishing Group; 2008. page 5115-23.
28. Loganathan S K, et al., Rare driver mutations in head and neck squamous cell carcinomas converge on NOTCH signaling. Science (80-) [Internet]. American Association for the Advancement of Science; 2020 [cited 2020 Mar. 18]; 367:1264-9. Available from: sciencemag.org/lookup/doi/10.1126/science.aax0902
29. Puram S V, et al., Single-Cell Transcriptomic Analysis of Primary and Metastatic Tumor Ecosystems in Head and Neck Cancer. Cell [Internet]. Elsevier; 2017 [cited 2018 May 7]; 171:1611-1624.e24. Available from: ncbi.nlm.nih.gov/pubmed/29198524
30. van der Flier L G, et al., Transcription Factor Achaete Scute-Like 2 Controls Intestinal Stem Cell Fate. Cell. 2009; 136:903-12.
31. Costoya J A, et al., Essential role of Plzf in maintenance of spermatogonial stem cells. Nat Genet. Nature Publishing Group; 2004; 36:653-9.
32. Buaas F W, et al., Plzf is required in adult male germ cells for stem cell self-renewal. Nat Genet. Nature Publishing Group; 2004; 36:647-52.
33. Zuo Q, et al., ASCL2 expression contributes to gastric tumor migration and invasion by downregulating miR223 and inducing EMT. Mol Med Rep. Spandidos Publications; 2018; 18:3751-9.
34. Shen H, et al., PLZF inhibits proliferation and metastasis of gallbladder cancer by regulating IFIT2 article. Cell Death Dis. Nature Publishing Group; 2018; 9:1-14.
35. Ran F A, et al., Genome engineering using the CRISPR-Cas9 system. Nat Protoc. 2013; 8:2281-308.
36. Joseph N M, et al., Genomic profiling of combined hepatocellular-cholangiocarcinoma reveals similar genetics to hepatocellular carcinoma. J Pathol [Internet]. John Wiley and Sons Ltd; 2019 [cited 2020 Apr. 7]; 248:164-78. Available from: http://www.ncbi.nlm.nih.gov/pubmed/30690729
37. Yeh I, et al., Targeted Genomic Profiling of Acral Melanoma. J Natl Cancer Inst [Internet]. 2019 [cited 2020 Apr. 7]; 111:1068-77. Available from: ncbi.nlm.nih.gov/pubmed/30657954
38. Hu Y, et al., Extreme limiting dilution analysis for comparing depleted and enriched populations in stem cell and other assays. J Immunol Methods [Internet]. Elsevier B.V.; 2009; 347:70-8. Available from: dx.doi.org/10.1016/j.jim.2009.06.008
39. Love M I, et al., Moderated estimation of fold change and dispersion for RNA-seq data with DESeq2. Genome Biol [Internet]. BioMed Central; 2014 [cited 2019 Mar. 6]; 15:550.
40. Mi H, et al., PANTHER in 2013: Modeling the evolution of gene function, and other gene attributes, in the context of phylogenetic trees. Nucleic Acids Res. 2013; 41.
41. Thomas P D, et al., PANTHER: A library of protein families and subfamilies indexed by function. Genome Res. 2003; 13:2129-41.

1. A method of genetically altering one or a plurality of primary keratinocytes comprising exposing the one or plurality of primary keratinocytes to one or a plurality of mutagens for a time period sufficient to mutate one or a plurality of wild-type genes such that one or a plurality of genome-edited primary keratinocytes deficient in expression of the one or plurality of wild-type genes are produced.

2. (canceled)

3. The method of claim 1, wherein the one or plurality of mutagens is a vector or an gene-modifying enzyme.

4. (canceled)

5. The method of claim 1 further comprising transfecting the one or plurality of primary keratinocytes with a plasmid comprising a nucleic acid sequence encoding the one or plurality of mutagens.

6. The method of claim 5, wherein the nucleic acid sequence encodes a Cas9 protein.

7. (canceled)

8. The method of claim 1 further comprising a step of selecting the one or plurality of genome-edited primary keratinocytes deficient in expression of the one or plurality of wild-type genes.

9. The method of claim 8, wherein the step of selecting is performed based on expression of a marker gene.

10. The method of claim 9, wherein the marker gene is a fluorescent reporter gene, an antibiotic resistance gene, or a surface protein-coding gene.

11.-13. (canceled)

14. The method of claim 1, wherein the one or plurality of genome-edited primary keratinocytes are deficient in expression of at least one of wild-type TP53, CDKN2A, and NOTCH1.

15. The method of claim 1, wherein the one or plurality of genome-edited primary keratinocytes are deficient in expression of wild-type TP53, CDKN2A, and NOTCH1.

16. A method of generating a population of one or a plurality of genome-edited keratinocytes comprising:
a) generating and selecting one or a plurality of genome-edited primary keratinocytes deficient in expression of one or a plurality of wild-type genes according to the method of claim 1; and
b) expanding the one or plurality of genome-edited primary keratinocytes into the population of one or plurality of genome-edited keratinocytes.

17. The method of claim 16, wherein the one or plurality of primary keratinocytes are primary human keratinocytes.

18.-21. (canceled)

22. A composition comprising one or a plurality of genome-edited keratinocytes that is deficient in expression of one or a plurality of wild-type genes chosen from TP53, CDKN2A and NOTCH1.

23.-25. (canceled)

26. The composition of claim 22 further comprising a top layer of cells and a bottom layer of tissue, the bottom layer of tissue comprising extracellular matrix protein and one or multiple cell types and the top layer of cells comprising the one or plurality of genome-edited keratinocytes.

27. The composition of claim 26, wherein the bottom layer of tissue comprises one or a plurality of fibroblasts.

28. The composition of claim 26 further comprising a hydrogel layer positioned underneath the bottom layer of tissue.

29. The composition of claim 28, wherein the hydrogel layer comprises one or a plurality of growth factors or functional fragments thereof.

30.-32. (canceled)

33. The composition further comprising at least a single insert comprising a biocompatible polymer and an inner wall defining an inner volume divided into an upper chamber and a bottom chamber, the upper chamber and bottom chamber positioned adjacently to one another and in fluid communication and separated by a ledge; the ledge positioned along an inner wall of the insert and defining a planar interface between the upper and bottom chambers, wherein the composition comprises a top layer of cells, a bottom layer of tissue and a hydrogel; each of the top layer of cells and bottom layer of tissue positioned in the upper chamber; and wherein the hydrogel is positioned in the bottom chamber; wherein the bottom layer of tissue comprises extracellular matrix protein and the top layer of cells comprises the one or plurality of genome-edited keratinocytes keratinocytes of claim 22.

34.-38. (canceled)

39. The composition of claim 33, wherein the composition is frozen at from about −20 to about −212 degrees Celsius.

40.-61. (canceled)

62. A method of isolating a primary keratinocytes from skin tissue or mucosa comprising culturing a primary keratinocyte to culture medium while simultaneously exposing one or more keratinocytes to one or a plurality of tissue samples comprising extracellular matrix (ECM).

63. (canceled)

64. The method of claim 62 or 63, wherein the skin is anal skin.

65. The method of claim 62, wherein the mucosa is oral mucosa, mucosa of the upper aerodigestive tract, esophageal mucosa, vaginal mucosa, or cervical mucosa.

66.-74. (canceled)

75. A method of screening a compound for biological activity comprising exposing one or a plurality of compounds to one or a plurality of genome-edited keratinocytes comprised in the composition of claim 22.

76. The composition of claim 26, wherein the bottom layer of tissue comprises one or a plurality of a combination of cells including fibroblasts, endothelium, CD45+ immune cells, pericytes, and/or myoepithelial cells.

77. (canceled)
